(19) United States
(12) Patent Application Publication
(10) Pub. No.: US 2026/0022382 A1
(43) Pub. Date: Jan. 22, 2026

(54) Compositions and Methods for Modulating Complement Factor B Expression

(71) Applicant: Ionis Pharmaceuticals, Inc., Carlsbad, CA (US)

(72) Inventors: Thazha P. Prakash, Carlsbad, CA (US); Punit P. Seth, Carlsbad, CA (US); Eric E. Swayze, Encinitas, CA (US); Tamar R. Grossman, La Jolla, CA (US); Michael L. McCaleb, La Jolla, CA (US); Andrew T. Watt, San Diego, CA (US); Susan M. Freier, San Diego, CA (US)

(73) Assignee: Ionis Pharmaceuticals, Inc., Carlsbad, CA (US)

(21) Appl. No.: 19/256,127

(22) Filed: Jun. 30, 2025

Related U.S. Application Data
(63) Continuation of application No. 18/320,948, filed on May 19, 2023, now abandoned.
Application No. 18/320,948 is a continuation of application No. 17/314,537, filed on May 7, 2021, now Pat. No. 11,732,265.
Application No. 17/314,537 is a continuation of application No. 16/357,018, filed on Mar. 18, 2019, now abandoned.
Application No. 16/357,018 is a division of application No. 15/307,526, now Pat. No. 10,280,423.
(60) Provisional application No. 61/987,471, filed on May 1, 2014.
(60) Provisional application No. 62/076,273, filed on Nov. 6, 2014.

(51) Int. Cl. C12N 15/113

(52) U.S. Cl. CPC C12N 15/1137; C12N 15/113; C12Y 304/21047; C12N 2310/11; C12N 2310/315; C12N 2310/321; C12N 2310/3341; C12N 2310/341; C12N 2310/346; C12N 2310/351

(57) ABSTRACT

The present embodiments provide methods, compounds, and compositions for treating, preventing, or ameliorating a disease associated with dysregulation of the complement alternative pathway by administering a Complement Factor B (CFB) specific inhibitor to a subject.

### SEQUENCE LISTING

The present application is being filed along with a Sequence Listing in electronic format. The Sequence Listing is provided as a file entitled BIOL0251USC1SEQ_ST25.txt created May 6, 2021, which is 204 kb in size. The information in the electronic format of the sequence listing is incorporated herein by reference in its entirety.

### FIELD

The present embodiments provide methods, compounds, and compositions for treating, preventing, or ameliorating a disease associated with dysregulation of the complement alternative pathway by administering a Complement Factor B (CFB) specific inhibitor to a subject.

### BACKGROUND

The complement system is part of the host innate immune system involved in lysing foreign cells, enhancing phagocytosis of antigens, clumping antigen-bearing agents, and attracting macrophages and neutrophils. The complement system is divided into three initiation pathways—the classical, lectin, and alternative pathways—that converge at component C3 to generate an enzyme complex known as C3 convertase, which cleaves C3 into C3a and C3b. C3b associates with C3 convertase mediated by CFB and results in generation of C5 convertase, which cleaves C5 into C5a and C5b, which initiates the membrane attack pathway resulting in the formation of the membrane attack complex (MAC) comprising components C5b, C6, C7, C8, and C9. The membrane-attack complex (MAC) forms transmembrane channels and disrupts the phospholipid bilayer of target cells, leading to cell lysis.

In the homeostatic state, the alternative pathway is continuously activated at a low “tickover” level as a result of activation of the alternative pathway by spontaneous hydrolysis of C3 and the production of C3b, which generates C5 convertase.

### SUMMARY

The complement system mediates innate immunity and plays an important role in normal inflammatory response to injury, but its dysregulation may cause severe injury. Activation of the alternative complement pathway beyond its constitutive “tickover” level can lead to unrestrained hyperactivity and manifest as diseases of complement dysregulation.

Certain embodiments provided herein relate to methods of treating, preventing, or ameliorating a disease associated with dysregulation of the complement alternative pathway in a subject by administration of a Complement Factor B (CFB) specific inhibitor. Several embodiments provided herein are drawn to a method of inhibiting expression of CFB in a subject having, or at risk of having, a disease associated with dysregulation of the complement alternative pathway by administering a CFB specific inhibitor to the subject. In certain embodiments, a method of reducing or inhibiting accumulation of C3 deposits in the eye of a subject having, or at risk of having, a disease associated with dysregulation of the complement alternative pathway comprises administering a CFB specific inhibitor to the subject. In several embodiments, a method of reducing or inhibiting accumulation of C3 deposits in the kidney of a subject having, or at risk of having, a disease associated with dysregulation of the complement alternative pathway comprises administering a CFB specific inhibitor to the subject.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated by reference for the portions of the document discussed herein, as well as in their entirety.

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21^{st} edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2^{nd} a Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

“2′-F nucleoside” refers to a nucleoside comprising a sugar comprising fluorine at the 2′ position. Unless otherwise indicated, the fluorine in a 2′-F nucleoside is in the ribo position (replacing the OH of a natural ribose).

“2′-O-methoxyethyl” (also 2′-MOE and 2′-O(CH₂)₂—OCH₃) refers to an O-methoxy-ethyl modification at the 2′ position of a furanose ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” means a nucleoside comprising a substituent at the 2′-position of the furanosyl ring other than H or OH. In certain embodiments, 2′ substituted nucleosides include nucleosides with bicyclic sugar modifications.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5 position. A 5-methylcytosine is a modified nucleobase.

“About” means within ±10% of a value. For example, if it is stated, “the compounds affected at least about 70% inhibition of CFB”, it is implied that CFB levels are inhibited within a range of 60% and 80%.

“Administration” or “administering” refers to routes of introducing an antisense compound provided herein to a subject to perform its intended function. An example of a route of administration that can be used includes, but is not limited to parenteral administration, such as subcutaneous, intravenous, or intramuscular injection or infusion.

“Alkyl,” as used herein, means a saturated straight or branched hydrocarbon radical containing up to twenty four carbon atoms. Examples of alkyl groups include without limitation, methyl, ethyl, propyl, butyl, isopropyl, n-hexyl, octyl, decyl, dodecyl and the like. Alkyl groups typically include from 1 to about 24 carbon atoms, more typically from 1 to about 12 carbon atoms (C1-C12 alkyl) with from 1 to about 6 carbon atoms being more preferred.

As used herein, “alkenyl,” means a straight or branched hydrocarbon chain radical containing up to twenty four carbon atoms and having at least one carbon-carbon double bond. Examples of alkenyl groups include without limitation, ethenyl, propenyl, butenyl, 1-methyl-2-buten-1-yl, dienes such as 1,3-butadiene and the like. Alkenyl groups typically include from 2 to about 24 carbon atoms, more typically from 2 to about 12 carbon atoms with from 2 to about 6 carbon atoms being more preferred. Alkenyl groups as used herein may optionally include one or more further substituent groups.

As used herein, “alkynyl,” means a straight or branched hydrocarbon radical containing up to twenty four carbon atoms and having at least one carbon-carbon triple bond. Examples of alkynyl groups include, without limitation, ethynyl, 1-propynyl, 1-butynyl, and the like. Alkynyl groups typically include from 2 to about 24 carbon atoms, more typically from 2 to about 12 carbon atoms with from 2 to about 6 carbon atoms being more preferred. Alkynyl groups as used herein may optionally include one or more further substituent groups.

As used herein, “acyl,” means a radical formed by removal of a hydroxyl group from an organic acid and has the general Formula —C(O)—X where X is typically aliphatic, alicyclic or aromatic. Examples include aliphatic carbonyls, aromatic carbonyls, aliphatic sulfonyls, aromatic sulfinyls, aliphatic sulfinyls, aromatic phosphates, aliphatic phosphates and the like. Acyl groups as used herein may optionally include further substituent groups.

As used herein, “alicyclic” means a cyclic ring system wherein the ring is aliphatic. The ring system can comprise one or more rings wherein at least one ring is aliphatic. Preferred alicyclics include rings having from about 5 to about 9 carbon atoms in the ring. Alicyclic as used herein may optionally include further substituent groups.

As used herein, “aliphatic” means a straight or branched hydrocarbon radical containing up to twenty four carbon atoms wherein the saturation between any two carbon atoms is a single, double or triple bond. An aliphatic group preferably contains from 1 to about 24 carbon atoms, more typically from 1 to about 12 carbon atoms with from 1 to about 6 carbon atoms being more preferred. The straight or branched chain of an aliphatic group may be interrupted with one or more heteroatoms that include nitrogen, oxygen, sulfur and phosphorus. Such aliphatic groups interrupted by heteroatoms include without limitation, polyalkoxys, such as polyalkylene glycols, polyamines, and polyimines. Aliphatic groups as used herein may optionally include further substituent groups.

As used herein, “alkoxy” means a radical formed between an alkyl group and an oxygen atom wherein the oxygen atom is used to attach the alkoxy group to a parent molecule. Examples of alkoxy groups include without limitation, methoxy, ethoxy, propoxy, isopropoxy, n-butoxy, sec-butoxy, tert-butoxy, n-pentoxy, neopentoxy, n-hexoxy and the like. Alkoxy groups as used herein may optionally include further substituent groups.

As used herein, “aminoalkyl” means an amino substituted C1-C12 alkyl radical. The alkyl portion of the radical forms a covalent bond with a parent molecule. The amino group can be located at any position and the aminoalkyl group can be substituted with a further substituent group at the alkyl and/or amino portions.

As used herein, “aralkyl” and “arylalkyl” mean an aromatic group that is covalently linked to a C1-C12 alkyl radical. The alkyl radical portion of the resulting aralkyl (or arylalkyl) group forms a covalent bond with a parent molecule. Examples include without limitation, benzyl, phenethyl and the like. Aralkyl groups as used herein may optionally include further substituent groups attached to the alkyl, the aryl or both groups that form the radical group.

As used herein, “aryl” and “aromatic” mean a mono- or polycyclic carbocyclic ring system radicals having one or more aromatic rings. Examples of aryl groups include without limitation, phenyl, naphthyl, tetrahydronaphthyl, indanyl, idenyl and the like. Preferred aryl ring systems have from about 5 to about 20 carbon atoms in one or more rings. Aryl groups as used herein may optionally include further substituent groups.

“Amelioration” refers to a lessening of at least one indicator, sign, or symptom of an associated disease, disorder, or condition. In certain embodiments, amelioration includes a delay or slowing in the progression of one or more indicators of a condition or disease. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means a single-stranded oligonucleotide having a nucleobase sequence that permits hybridization to a corresponding region or segment of a target nucleic acid.

“Base complementarity” refers to the capacity for the precise base pairing of nucleobases of an antisense oligonucleotide with corresponding nucleobases in a target nucleic acid (i.e., hybridization), and is mediated by Watson-Crick, Hoogsteen or reversed Hoogsteen hydrogen binding between corresponding nucleobases.

“Bicyclic sugar moiety” means a modified sugar moiety comprising a 4 to 7 membered ring (including but not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments the 4 to 7 membered ring is a furanosyl. In certain such embodiments, the bridge connects the 2′-carbon and the 4′-carbon of the furanosyl.

“Bicyclic nucleic acid” or “BNA” or “BNA nucleosides” means nucleic acid monomers having a bridge connecting two carbon atoms between the 4′ and 2′position of the nucleoside sugar unit, thereby forming a bicyclic sugar. Examples of such bicyclic sugar include, but are not limited to A) α-L-Methyleneoxy (4′-CH₂—O-2′) LNA, (B) β-D-Methyleneoxy (4′-CH₂—O-2′) LNA, (C) Ethyleneoxy (4′-(CH₂)₂—O-2′) LNA, (D) Aminooxy (4′-CH₂—O—N(R)-2′) LNA and (E) Oxyamino (4′-CH₂—N(R)—O-2′) LNA, as depicted below.

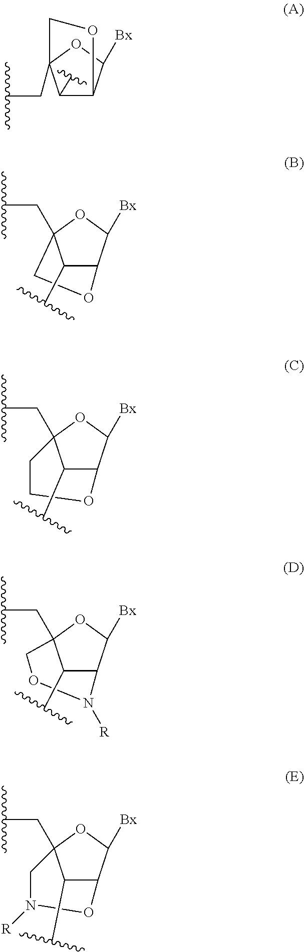

As used herein, LNA compounds include, but are not limited to, compounds having at least one bridge between the 4′ and the 2′ position of the sugar wherein each of the bridges independently comprises 1 or from 2 to 4 linked groups independently selected from —[C(R₁)(R₂)]ₙ—, —C(R₁)═C(R₂)—, —C(R₁)═N—, —C(═NR₁)—, —C(═O)—, —C(═S)—, —O—, —Si(R₁)₂—, —S(═O)ᵣ, and —N(R₁)—; wherein: x is 0, 1, or 2; n is 1, 2, 3, or 4; each R₁ and R₂ is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, a heterocycle radical, a substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

Examples of 4′-2′ bridging groups encompassed within the definition of LNA include, but are not limited to one of formulae: —[C(R₁)(R₂)]ₙ—, —[C(R₁)(R₂)]ₙ—O—, —C(R₁R₂)—N(R₁)—O— or —C(R₁R₂)—O—N(R₁)—. Furthermore, other bridging groups encompassed with the definition of LNA are 4′-CH₂-2′, 4′-(CH₂)₂-2′, 4′-(CH₂)₃-2′, 4′—CH₂—O-2′, 4′-(CH₂)₂—O-2′, 4′—CH₂—O—N(R₁)-2′ and 4′-CH₂—N(R₁)—O-2′- bridges, wherein each R₁ and R₂ is, independently, H, a protecting group or C₁-C₁₂ alkyl.

Also included within the definition of LNA according to the invention are LNAs in which the 2′-hydroxyl group of the ribosyl sugar ring is connected to the 4′ carbon atom of the sugar ring, thereby forming a methyleneoxy (4′-CH₂—O-2′) bridge to form the bicyclic sugar moiety. The bridge can also be a methylene (—CH₂—) group connecting the 2′ oxygen atom and the 4′ carbon atom, for which the term methyleneoxy (4′-CH₂—O-2′) LNA is used. Furthermore; in the case of the bicylic sugar moiety having an ethylene bridging group in this position, the term ethyleneoxy (4′-CH₂CH₂—O-2′) LNA is used. α-L-methyleneoxy (4′-CH₂—O-2′), an isomer of methyleneoxy (4′-CH₂—O-2′) LNA is also encompassed within the definition of LNA, as used herein.

“Cap structure” or “terminal cap moiety” means chemical modifications, which have been incorporated at either terminus of an antisense compound.

“Carbohydrate” means a naturally occurring carbohydrate, a modified carbohydrate, or a carbohydrate derivative.

“Carbohydrate cluster” means a compound having one or more carbohydrate residues attached to a scaffold or linker group. (see, e.g., Maier et al., “Synthesis of Antisense Oligonucleotides Conjugated to a Multivalent Carbohydrate Cluster for Cellular Targeting,” Bioconjugate Chemistry, 2003, (14): 18-29, which is incorporated herein by reference in its entirety, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, (47): 5798-5808, for examples of carbohydrate conjugate clusters).

“Carbohydrate derivative” means any compound which may be synthesized using a carbohydrate as a starting material or intermediate.

“cEt” or “constrained ethyl” means a bicyclic sugar moiety comprising a bridge connecting the 4′-carbon and the 2′-carbon, wherein the bridge has the formula: 4′-CH(CH₃)—O-2′.

“Chemical modification” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group that is capable of being split under physiological conditions. In certain embodiments, a cleavable moiety is cleaved inside a cell or sub-cellular compartments, such as a lysosome. In certain embodiments, a cleavable moiety is cleaved by endogenous enzymes, such as nucleases. In certain embodiments, a cleavable moiety comprises a group of atoms having one, two, three, four, or more than four cleavable bonds.

“Conjugate” or “conjugate group” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

“conjugate linker” or “linker” in the context of a conjugate group means a portion of a conjugate group comprising any atom or group of atoms and which covalently link (1) an oligonucleotide to another portion of the conjugate group or (2) two or more portions of the conjugate group.

Conjugate groups are shown herein as radicals, providing a bond for forming covalent attachment to an oligomeric compound such as an antisense oligonucleotide. In certain embodiments, the point of attachment on the oligomeric compound is the 3′-oxygen atom of the 3-hydroxyl group of the 3′ terminal nucleoside of the oligomeric compound. In certain embodiments the point of attachment on the oligomeric compound is the 5′-oxygen atom of the 5-hydroxyl group of the 5′ terminal nucleoside of the oligomeric compound. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and a carbohydrate cluster portion, such as a GalNAc cluster portion. Such carbohydrate cluster portion comprises: a targeting moiety and, optionally, a conjugate linker. In certain embodiments, the carbohydrate cluster portion is identified by the number and identity of the ligand. For example, in certain embodiments, the carbohydrate cluster portion comprises 3 GalNAc groups and is designated “GalNAc3”. In certain embodiments, the carbohydrate cluster portion comprises 4 GalNAc groups and is designated “GalNAc4”. Specific carbohydrate cluster portions (having specific tether, branching and conjugate linker groups) are described herein and designated by Roman numeral followed by subscript “a”. Accordingly “GalNac3-1ₐ” refers to a specific carbohydrate cluster portion of a conjugate group having 3 GalNac groups and specifically identified tether, branching and linking groups. Such carbohydrate cluster fragment is attached to an oligomeric compound via a cleavable moiety, such as a cleavable bond or cleavable nucleoside.

“Conjugate compound” means any atoms, group of atoms, or group of linked atoms suitable for use as a conjugate group. In certain embodiments, conjugate compounds may possess or impart one or more properties, including, but not limited to pharmacodynamic, pharmacokinetic, binding, absorption, cellular distribution, cellular uptake, charge and/or clearance properties.

“Constrained ethyl nucleoside” (also cEt nucleoside) means a nucleoside comprising a bicyclic sugar moiety comprising a 4′-CH(CH₃)—O-2′ bridge.

“Complement Factor B (CFB)” means any nucleic acid or protein of CFB. “CFB nucleic acid” means any nucleic acid encoding CFB. For example, in certain embodiments, a CFB nucleic acid includes a DNA sequence encoding CFB, an RNA sequence transcribed from DNA encoding CFB (including genomic DNA comprising introns and exons), including a non-protein encoding (i.e. non-coding) RNA sequence, and an mRNA sequence encoding CFB. “CFB mRNA” means an mRNA encoding a CFB protein.

“CFB specific inhibitor” refers to any agent capable of specifically inhibiting CFB RNA and/or CFB protein expression or activity at the molecular level. For example, CFB specific inhibitors include nucleic acids (including antisense compounds), peptides, antibodies, small molecules, and other agents capable of inhibiting the expression of CFB RNA and/or CFB protein.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Complementarity” means the capacity for pairing between nucleobases of a first nucleic acid and a second nucleic acid.

“Comprise,” “comprises” and “comprising” will be understood to imply the inclusion of a stated step or element or group of steps or elements but not the exclusion of any other step or element or group of steps or elements.

“Contiguous nucleobases” means nucleobases immediately adjacent to each other.

“Deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

“Deoxyribonucleotide” means a nucleotide having a hydrogen at the 2′ position of the sugar portion of the nucleotide. Deoxyribonucleotides may be modified with any of a variety of substituents.

“Designing” or “Designed to” refer to the process of designing an oligomeric compound that specifically hybridizes with a selected nucleic acid molecule.

“Differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Double-stranded” refers to two separate oligomeric compounds that are hybridized to one another. Such double stranded compounds may have one or more or non-hybridizing nucleosides at one or both ends of one or both strands (overhangs) and/or one or more internal non-hybridizing nucleosides (mismatches) provided there is sufficient complementarity to maintain hybridization under physiologically relevant conditions.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Expression” includes all the functions by which a gene's coded information is converted into structures present and operating in a cell. Such structures include, but are not limited to the products of transcription and translation.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Furanosyl” means a structure comprising a 5-membered ring comprising four carbon atoms and one oxygen atom.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

“Halo” and “halogen,” mean an atom selected from fluorine, chlorine, bromine and iodine.

“Heteroaryl,” and “heteroaromatic,” mean a radical comprising a mono- or poly-cyclic aromatic ring, ring system or fused ring system wherein at least one of the rings is aromatic and includes one or more heteroatoms. Heteroaryl is also meant to include fused ring systems including systems where one or more of the fused rings contain no heteroatoms. Heteroaryl groups typically include one ring atom selected from sulfur, nitrogen or oxygen. Examples of heteroaryl groups include without limitation, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl and the like. Heteroaryl radicals can be attached to a parent molecule directly or through a linking moiety such as an aliphatic group or hetero atom. Heteroaryl groups as used herein may optionally include further substituent groups.

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Identifying an animal having, or at risk for having, a disease, disorder and/or condition” means identifying an animal having been diagnosed with the disease, disorder and/or condition or identifying an animal predisposed to develop the disease, disorder and/or condition. Such identification may be accomplished by any method including evaluating an individual's medical history and standard clinical tests or assessments.

“Immediately adjacent” means there are no intervening elements between the immediately adjacent elements.

“Individual” means a human or non-human animal selected for treatment or therapy.

“Inhibiting the expression or activity” refers to a reduction, blockade of the expression or activity and does not necessarily indicate a total elimination of expression or activity.

“Internucleoside linkage” refers to the chemical bond between nucleosides.

“Internucleoside neutral linking group” means a neutral linking group that directly links two nucleosides.

“Internucleoside phosphorus linking group” means a phosphorus linking group that directly links two nucleosides.

“Lengthened” antisense oligonucleotides are those that have one or more additional nucleosides relative to an antisense oligonucleotide disclosed herein.

“Linkage motif” means a pattern of linkage modifications in an oligonucleotide or region thereof. The nucleosides of such an oligonucleotide may be modified or unmodified. Unless otherwise indicated, motifs herein describing only linkages are intended to be linkage motifs. Thus, in such instances, the nucleosides are not limited.

“Linked deoxynucleoside” means a nucleic acid base (A, G, C, T, U) substituted by deoxyribose linked by a phosphate ester to form a nucleotide.

“Linked nucleosides” means adjacent nucleosides linked together by an internucleoside linkage.

“Locked nucleic acid nucleoside” or “LNA” means a nucleoside comprising a bicyclic sugar moiety comprising a 4′-CH2-O-2′bridge.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases adenine (A) and guanine (G), and the pyrimidine bases thymine (T), cytosine (C) and uracil (U).

“Modified nucleoside” means a nucleoside having, independently, a modified sugar moiety and/or modified nucleobase.

“Modified nucleotide” means a nucleotide having, independently, a modified sugar moiety, modified internucleoside linkage, or modified nucleobase.

“Modified oligonucleotide” means an oligonucleotide comprising at least one modified internucleoside linkage, a modified sugar, and/or a modified nucleobase.

“Modified sugar” means substitution and/or any change from a natural sugar moiety.

“Modulating” refers to changing or adjusting a feature in a cell, tissue, organ or organism. For example, modulating CFB mRNA can mean to increase or decrease the level of CFB mRNA and/or CFB protein in a cell, tissue, organ or organism. A “modulator” effects the change in the cell, tissue, organ or organism. For example, a CFB antisense compound can be a modulator that decreases the amount of CFB mRNA and/or CFB protein in a cell, tissue, organ or organism.

“Monomer” refers to a single unit of an oligomer. Monomers include, but are not limited to, nucleosides and nucleotides, whether naturally occurring or modified.

“Mono or polycyclic ring system” is meant to include all ring systems selected from single or polycyclic radical ring systems wherein the rings are fused or linked and is meant to be inclusive of single and mixed ring systems individually selected from aliphatic, alicyclic, aryl, heteroaryl, aralkyl, arylalkyl, heterocyclic, heteroaryl, hetero¬aromatic and heteroarylalkyl. Such mono and poly cyclic structures can contain rings that each have the same level of saturation or each, independently, have varying degrees of saturation including fully saturated, partially saturated or fully unsaturated. Each ring can comprise ring atoms selected from C, N, O and S to give rise to hetero¬cyclic rings as well as rings comprising only C ring atoms which can be present in a mixed motif such as for example benzimidazole wherein one ring has only carbon ring atoms and the fused ring has two nitrogen atoms. The mono or polycyclic ring system can be further substituted with substituent groups such as for example phthalimide which has two ═O groups attached to one of the rings. Mono or polycyclic ring systems can be attached to parent molecules using various strategies such as directly through a ring atom, fused through multiple ring atoms, through a substituent group or through a bifunctional linking moiety.

“Motif” means the pattern of unmodified and modified nucleosides in an antisense compound.

“Natural sugar moiety” means a sugar moiety found in DNA (2′-H) or RNA (2′-OH).

“Naturally occurring internucleoside linkage” means a 3′ to 5′ phosphodiester linkage.

“Neutral linking group” means a linking group that is not charged. Neutral linking groups include without limitation phospho¬triesters, methylphosphonates, MMI (—CH2-N(CH3)-O—), amide-3 (—CH2-C(═O)—N(H)—), amide-4 (—CH2-N(H)—C(═O)—), formacetal (—O—CH2-O—), and thioformacetal (—S—CH2-O—). Further neutral linking groups include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook Eds. ACS Symposium Series 580; Chapters 3 and 4, (pp. 40-65)). Further neutral linking groups include nonionic linkages comprising mixed N, O, S and CH2 component parts.

“Non-complementary nucleobase” refers to a pair of nucleobases that do not form hydrogen bonds with one another or otherwise support hybridization.

“Non-internucleoside neutral linking group” means a neutral linking group that does not directly link two nucleosides. In certain embodiments, a non-internucleoside neutral linking group links a nucleoside to a group other than a nucleoside. In certain embodiments, a non-internucleoside neutral linking group links two groups, neither of which is a nucleoside.

“Non-internucleoside phosphorus linking group” means a phosphorus linking group that does not directly link two nucleosides. In certain embodiments, a non-internucleoside phosphorus linking group links a nucleoside to a group other than a nucleoside. In certain embodiments, a non-internucleoside phosphorus linking group links two groups, neither of which is a nucleoside.

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase modification motif” means a pattern of modifications to nucleobases along an oligonucleotide. Unless otherwise indicated, a nucleobase modification motif is independent of the nucleobase sequence.

“Nucleobase sequence” means the order of contiguous nucleobases independent of any sugar, linkage, and/or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O—or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system. “Mimetic” refers to groups that are substituted for a sugar, a nucleobase, and/or internucleoside linkage. Generally, a mimetic is used in place of the sugar or sugar-internucleoside linkage combination, and the nucleobase is maintained for hybridization to a selected target.

“Nucleoside motif” means a pattern of nucleoside modifications in an oligonucleotide or a region thereof. The linkages of such an oligonucleotide may be modified or unmodified. Unless otherwise indicated, motifs herein describing only nucleosides are intended to be nucleoside motifs. Thus, in such instances, the linkages are not limited.

“Nucleotide” means a nucleoside having a phosphate group covalently linked to the sugar portion of the nucleoside.

“Oligomeric compound” means a polymer of linked monomeric subunits which is capable of hybridizing to at least a region of a nucleic acid molecule.

“Oligonucleoside” means an oligonucleotide in which the internucleoside linkages do not contain a phosphorus atom.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutical composition” means a mixture of substances suitable for administering to an individual. For example, a pharmaceutical composition may comprise one or more active pharmaceutical agents and a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Phosphorus linking group” means a linking group comprising a phosphorus atom. Phosphorus linking groups include without limitation groups having the formula:

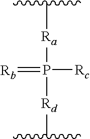

wherein:
   Rₐ and R_{d} are each, independently, O, S, CH₂, NH, or NJ₁ wherein J₁ is C₁-C₆ alkyl or substituted C₁-C₆ alkyl;
   R_{b}, is O or S;
   R_{c} is OH, SH, C₁-C₆ alkyl, substituted C₁-C₆ alkyl, C₁-C₆ alkoxy, substituted C₁-C₆ alkoxy, amino or substituted amino; and
   J₁ is R_{b} is O or S.
Phosphorus linking groups include without limitation, phosphodiester, phosphorothioate, phosphorodithioate, phosphonate, phosphoramidate, phosphorothioamidate, thionoalkylphosphonate, phosphotriesters, thionoalkylphosphotriester and boranophosphate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound

“Prevent” refers to delaying or forestalling the onset, development or progression of a disease, disorder, or condition for a period of time from minutes to indefinitely. Prevent also means reducing the risk of developing a disease, disorder, or condition.

“Prodrug” means an inactive or less active form of a compound which, when administered to a subject, is metabolized to form the active, or more active, compound (e.g., drug).

“Prophylactically effective amount” refers to an amount of a pharmaceutical agent that provides a prophylactic or preventative benefit to an animal.

“Protecting group” means any compound or protecting group known to those having skill in the art. Non-limiting examples of protecting groups may be found in “Protective Groups in Organic Chemistry”, T. W. Greene, P. G. M. Wuts, ISBN 0-471-62301-6, John Wiley & Sons, Inc, New York, which is incorporated herein by reference in its entirety.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides may be modified with any of a variety of substituents.

“RISC based antisense compound” means an antisense compound wherein at least some of the antisense activity of the antisense compound is attributable to the RNA Induced Silencing Complex (RISC).

“RNase H based antisense compound” means an antisense compound wherein at least some of the antisense activity of the antisense compound is attributable to hybridization of the antisense compound to a target nucleic acid and subsequent cleavage of the target nucleic acid by RNase H.

“Segments” are defined as smaller or sub-portions of regions within a target nucleic acid.

“Separate regions” means portions of an oligonucleotide wherein the chemical modifications or the motif of chemical modifications of any neighboring portions include at least one difference to allow the separate regions to be distinguished from one another.

“Sequence motif” means a pattern of nucleobases arranged along an oligonucleotide or portion thereof. Unless otherwise indicated, a sequence motif is independent of chemical modifications and thus may have any combination of chemical modifications, including no chemical modifications.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Sites,” as used herein, are defined as unique nucleobase positions within a target nucleic acid.

“Slows progression” means decrease in the development of the said disease.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Stringent hybridization conditions” or “stringent conditions” refer to conditions under which an oligomeric compound will hybridize to its target sequence, but to a minimal number of other sequences.

“Subject” means a human or non-human animal selected for treatment or therapy.

“Substituent” and “substituent group,” means an atom or group that replaces the atom or group of a named parent compound. For example a substituent of a modified nucleoside is any atom or group that differs from the atom or group found in a naturally occurring nucleoside (e.g., a modified 2′-substuent is any atom or group at the 2′-position of a nucleoside other than H or OH). Substituent groups can be protected or unpro¬tected. In certain embodiments, compounds of the present disclosure have substituents at one or at more than one position of the parent compound. Substituents may also be further substituted with other substituent groups and may be attached directly or via a linking group such as an alkyl or hydro¬carbyl group to a parent compound.

Likewise, as used herein, “substituent” in reference to a chemical functional group means an atom or group of atoms that differs from the atom or a group of atoms normally present in the named functional group. In certain embodiments, a substituent replaces a hydrogen atom of the functional group (e.g., in certain embodiments, the substituent of a substituted methyl group is an atom or group other than hydrogen which replaces one of the hydrogen atoms of an unsubstituted methyl group). Unless otherwise indicated, groups amenable for use as substituents include without limitation, halogen, hydroxyl, alkyl, alkenyl, alkynyl, acyl (—C¬(O)¬Raa), carboxyl (—C(O)O-Raa), aliphatic groups, ali-cyclic groups, alkoxy, substituted oxy (—O-Raa), aryl, aralkyl, heterocyclic radical, hetero¬aryl, hetero-arylalkyl, amino (N(Rbb)¬(Rcc)), imino(=NRbb), amido (C(O)N¬(Rbb)(Rcc) or N(Rbb)C(O)Raa), azido (—N3), nitro (NO2), cyano (—CN), carbamido (OC(O)N(Rbb)(Rcc) or N(Rbb)¬C(O)¬ORaa), ureido (N(Rbb)C(O)¬N(Rbb)(Rcc)), thioureido (N(Rbb)C¬¬¬(S)N(Rbb)¬(Rcc)), guanidinyl (N(Rbb)¬C(═NRbb)-N(Rbb)(Rcc)), amidinyl (C(═NRbb)¬¬N(Rbb)(Rcc) or N(Rbb)C(═NRbb)(Raa)), thiol (—SRbb), sulfinyl (S(O)Rbb), sulfonyl (—S(O)₂Rbb) and sulfonamidyl (—S(O)2N(Rbb)(Rcc) or N(Rbb)¬S¬¬(O)2Rbb). Wherein each Raa, Rbb and Rcc is, independently, H, an optionally linked chemical functional group or a further substituent group with a preferred list including without limitation, alkyl, alkenyl, alkynyl, aliphatic, alkoxy, acyl, aryl, aralkyl, heteroaryl, alicyclic, heterocyclic and hetero¬aryl¬alkyl. Selected substituents within the compounds described herein are present to a recursive degree.

“Substituted sugar moiety” means a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties.

“Sugar moiety” means a naturally occurring sugar moiety or a modified sugar moiety of a nucleoside.

“Sugar motif” means a pattern of sugar modifications in an oligonucleotide or a region thereof.

“Sugar surrogate” means a structure that does not comprise a furanosyl and that is capable of replacing the naturally occurring sugar moiety of a nucleoside, such that the resulting nucleoside sub-units are capable of linking together and/or linking to other nucleosides to form an oligomeric compound which is capable of hybridizing to a complementary oligomeric compound. Such structures include rings comprising a different number of atoms than furanosyl (e.g., 4, 6, or 7-membered rings); replacement of the oxygen of a furanosyl with a non-oxygen atom (e.g., carbon, sulfur, or nitrogen); or both a change in the number of atoms and a replacement of the oxygen. Such structures may also comprise substitutions corresponding to those described for substituted sugar moieties (e.g., 6-membered carbocyclic bicyclic sugar surrogates optionally comprising additional substituents). Sugar surrogates also include more complex sugar replacements (e.g., the non-ring systems of peptide nucleic acid). Sugar surrogates include without limitation morpholinos, cyclohexenyls and cyclohexitols.

“Target” refers to a protein, the modulation of which is desired.

“Target gene” refers to a gene encoding a target.

“Targeting” means the process of design and selection of an antisense compound that will specifically hybridize to a target nucleic acid and induce a desired effect.

“Target nucleic acid,” “target RNA,” “target RNA transcript” and “nucleic acid target” all mean a nucleic acid capable of being targeted by antisense compounds.

“Target region” means a portion of a target nucleic acid to which one or more antisense compounds is targeted.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Terminal group” means one or more atom attached to either, or both, the 3′ end or the 5′ end of an oligonucleotide. In certain embodiments a terminal group is a conjugate group. In certain embodiments, a terminal group comprises one or more terminal group nucleosides.

“Terminal internucleoside linkage” means the linkage between the last two nucleosides of an oligonucleotide or defined region thereof.

“Therapeutically effective amount” means an amount of a pharmaceutical agent that provides a therapeutic benefit to an individual.

“Treat” refers to administering a pharmaceutical composition to an animal in order to effect an alteration or improvement of a disease, disorder, or condition in the animal. In certain embodiments, one or more pharmaceutical compositions can be administered to the animal.

“Unmodified” nucleobases mean the purine bases adenine (A) and guanine (G), and the pyrimidine bases thymine (T), cytosine (C) and uracil (U).

“Unmodified nucleotide” means a nucleotide composed of naturally occurring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

Certain embodiments provide methods, compounds and compositions for inhibiting Complement Factor B (CFB) expression.

Certain embodiments provide antisense compounds targeted to a CFB nucleic acid. In certain embodiments, the CFB nucleic acid has the sequence set forth in GENBANK Accession No. NM_001710.5 (incorporated herein as SEQ ID NO: 1), GENBANK Accession No. NT_007592.15 truncated from nucleotides 31852000 to 31861000 (incorporated herein as SEQ ID NO: 2), GENBANK Accession No NW_001116486.1 truncated from nucleotides 536000 to 545000 (incorporated herein as SEQ ID NO: 3), GENBANK Accession No. XM_001113553.2 (incorporated herein as SEQ ID NO: 4), or GENBANK Accession No. NM_008198.2 (incorporated herein as SEQ ID NO: 5).

Certain embodiments provide a compound comprising a modified oligonucleotide and a conjugate group, wherein the modified oligonucleotide consists of 10 to 30 linked nucleosides and has a nucleobase sequence comprising at least 8 contiguous nucleobases of any of the nucleobase sequences of SEQ ID NOs: 6-808.

Certain embodiments provide a compound comprising a modified oligonucleotide and a conjugate group, wherein the modified oligonucleotide consists of 10 to 30 linked nucleosides and has a nucleobase sequence comprising at least 9 contiguous nucleobases of any of the nucleobase sequences of SEQ ID NOs: 6-808.

Certain embodiments provide a compound comprising a modified oligonucleotide and a conjugate group, wherein the modified oligonucleotide consists of 10 to 30 linked nucleosides and has a nucleobase sequence comprising at least 10 contiguous nucleobases of any of the nucleobase sequences of SEQ ID NOs: 6-808.

Certain embodiments provide a compound comprising a modified oligonucleotide and a conjugate group, wherein the modified oligonucleotide consists of 10 to 30 linked nucleosides and has a nucleobase sequence comprising at least 11 contiguous nucleobases of any of the nucleobase sequences of SEQ ID NOs: 6-808.

Certain embodiments provide a compound comprising a modified oligonucleotide and a conjugate group, wherein the modified oligonucleotide consists of 10 to 30 linked nucleosides and has a nucleobase sequence comprising at least 12 contiguous nucleobases of any of the nucleobase sequences of SEQ ID NOs: 6-808.

Certain embodiments provide a compound comprising a modified oligonucleotide and a conjugate group, wherein the modified oligonucleotide consists of 10 to 30 linked nucleosides and has a nucleobase sequence comprising the nucleobase sequence of any one of SEQ ID NOs: 6-808.

Certain embodiments provide a compound comprising a modified oligonucleotide and a conjugate group, wherein the modified oligonucleotide consists of the nucleobase sequence of any one of SEQ ID NOs: 6-808.

Certain embodiments provide a compound comprising a modified oligonucleotide and a conjugate group, wherein the modified oligonucleotide consists of 10 to 30 linked nucleosides complementary within nucleobases 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, or 2616-2631 of SEQ ID NO: 1, and wherein said modified oligonucleotide is at least 85%, at least 90%, at least 95%, or 100% complementary to SEQ ID NO: 1.

Certain embodiments provide a compound comprising a modified oligonucleotide and a conjugate group, wherein the modified oligonucleotide consists of 10 to 30 linked nucleosides having a nucleobase sequence comprising a portion of at least 8 contiguous nucleobases complementary to an equal length portion of nucleobases 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, or 2616-2631 of SEQ ID NO:1, and wherein the nucleobase sequence of the modified oligonucleotide is at least 85%, at least 90%, at least 95%, or 100% complementary to SEQ ID NO: 1.

Certain embodiments provide a compound comprising a modified oligonucleotide and a conjugate group, wherein the modified oligonucleotide consists of 10 to 30 linked nucleosides complementary within nucleobases 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838, 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, or 7846-7862 of SEQ ID NO: 2, and wherein said modified oligonucleotide is at least 85%, at least 90%, at least 95%, or 100% complementary to SEQ ID NO: 2.

Certain embodiments provide a compound comprising a modified oligonucleotide and a conjugate group, wherein the modified oligonucleotide consists of 10 to 30 linked nucleosides having a nucleobase sequence comprising a portion of at least 8 contiguous nucleobases complementary to an equal length portion of nucleobases 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838, 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, and 7846-7862 of SEQ ID NO: 2, and wherein the nucleobase sequence of the modified oligonucleotide is at least 85%, at least 90%, at least 95%, or 100% complementary to SEQ ID NO: 2.

In certain embodiments, antisense compounds or oligonucleotides target a region of a CFB nucleic acid. In certain embodiments, such compounds or oligonucleotides targeted to a region of a CFB nucleic acid have a contiguous nucleobase portion that is complementary to an equal length nucleobase portion of the region. For example, the portion can be at least an 8, 9, 10, 11, 12, 13, 14, 15, or 16 contiguous nucleobase portion complementary to an equal length portion of a region recited herein. In certain embodiments, a compound comprises or consists of a conjugate and a modified oligonucleotide targeting any of the following nucleotide regions of SEQ ID NO: 1: 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, and 2616-2631.

In certain embodiments, antisense compounds or oligonucleotides target a region of a CFB nucleic acid. In certain embodiments, such compounds or oligonucleotides targeted to a region of a CFB nucleic acid have a contiguous nucleobase portion that is complementary to an equal length nucleobase portion of the region. For example, the portion can be at least an 8, 9, 10, 11, 12, 13, 14, 15, or 16 contiguous nucleobase portion complementary to an equal length portion of a region recited herein. In certain embodiments, a compound comprises or consists of a conjugate and a modified oligonucleotide targeting the following nucleotide regions of SEQ ID NO: 2: 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838, 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, and 7846-7862.

In certain embodiments, a compound comprises or consists of a conjugate and a modified oligonucleotide targeting the 3′UTR of a CFB nucleic acid. In certain aspects, the modified oligonucleotide targets within nucleotides 2574-2626 of a CFB nucleic acid having the nucleobase sequence of SEQ ID NO: 1. In certain aspects, the modified oligonucleotide has at least an 8, 9, 10, 11, 12, 13, 14, 15, or 16 contiguous nucleobase portion complementary to an equal length portion within nucleotides 2574-2626 of a CFB nucleic acid having the nucleobase sequence of SEQ ID NO: 1.

In certain embodiments, a compound comprises or consists of a conjugate and a modified oligonucleotide targeting a region of a CFB nucleic acid having the nucleobase sequence of SEQ ID NO: 1 within nucleobases 2457-2631, 2457-2472, 2457-2474, 2457-2476, 2457-2566, 2457-2570, 2457-2571, 2457-2572, 2457-2573, 2457-2574, 2457-2575, 2457-2576, 2457-2577, 2457-2578, 2457-2579, 2457-2580, 2457-2581, 2457-2582, 2457-2583, 2457-2584, 2457-2585, 2457-2586, 2457-2587, 2457-2588, 2457-2589, 2457-2590, 2457-2591, 2457-2592, 2457-2593, 2457-2594, 2457-2595, 2457-2596, 2457-2597, 2457-2598, 2457-2599, 2457-2600, 2457-2601, 2457-2602, 2457-2603, 2457-2604, 2457-2605, 2457-2606, 2457-2607, 2457-2608, 2457-2609, 2457-2610, 2457-2611, 2457-2612, 2457-2613, 2457-2614, 2457-2615, 2457-2616, 2457-2617, 2457-2618, 2457-2619, 2457-2620, 2457-2621, 2457-2622, 2457-2623, 2457-2624, 2457-2625, 2457-2626, 2457-2627, 2457-2628, 2457-2629, 2457-2630, 2457-2631, 2459-2474, 2459-2476, 2459-2566, 2459-2570, 2459-2571, 2459-2572, 2459-2573, 2459-2574, 2459-2575, 2459-2576, 2459-2577, 2459-2578, 2459-2579, 2459-2580, 2459-2581, 2459-2582, 2459-2583, 2459-2584, 2459-2585, 2459-2586, 2459-2587, 2459-2588, 2459-2589, 2459-2590, 2459-2591, 2459-2592, 2459-2593, 2459-2594, 2459-2595, 2459-2596, 2459-2597, 2459-2598, 2459-2599, 2459-2600, 2459-2601, 2459-2602, 2459-2603, 2459-2604, 2459-2605, 2459-2606, 2459-2607, 2459-2608, 2459-2609, 2459-2610, 2459-2611, 2459-2612, 2459-2613, 2459-2614, 2459-2615, 2459-2616, 2459-2617, 2459-2618, 2459-2619, 2459-2620, 2459-2621, 2459-2622, 2459-2623, 2459-2624, 2459-2625, 2459-2626, 2459-2627, 2459-2628, 2459-2629, 2459-2630, 2459-2631, 2461-2476, 2461-2566, 2461-2570, 2461-2571, 2461-2572, 2461-2573, 2461-2574, 2461-2575, 2461-2576, 2461-2577, 2461-2578, 2461-2579, 2461-2580, 2461-2581, 2461-2582, 2461-2583, 2461-2584, 2461-2585, 2461-2586, 2461-2587, 2461-2588, 2461-2589, 2461-2590, 2461-2591, 2461-2592, 2461-2593, 2461-2594, 2461-2595, 2461-2596, 2461-2597, 2461-2598, 2461-2599, 2461-2600, 2461-2601, 2461-2602, 2461-2603, 2461-2604, 2461-2605, 2461-2606, 2461-2607, 2461-2608, 2461-2609, 2461-2610, 2461-2611, 2461-2612, 2461-2613, 2461-2614, 2461-2615, 2461-2616, 2461-2617, 2461-2618, 2461-2619, 2461-2620, 2461-2621, 2461-2622, 2461-2623, 2461-2624, 2461-2625, 2461-2626, 2461-2627, 2461-2628, 2461-2629, 2461-2630, 2461-2631, 2551-2566, 2551-2570, 2551-2571, 2551-2572, 2551-2573, 2551-2574, 2551-2575, 2551-2576, 2551-2577, 2551-2578, 2551-2579, 2551-2580, 2551-2581, 2551-2582, 2551-2583, 2551-2584, 2551-2585, 2551-2586, 2551-2587, 2551-2588, 2551-2589, 2551-2590, 2551-2591, 2551-2592, 2551-2593, 2551-2594, 2551-2595, 2551-2596, 2551-2597, 2551-2598, 2551-2599, 2551-2600, 2551-2601, 2551-2602, 2551-2603, 2551-2604, 2551-2605, 2551-2606, 2551-2607, 2551-2608, 2551-2609, 2551-2610, 2551-2611, 2551-2612, 2551-2613, 2551-2614, 2551-2615, 2551-2616, 2551-2617, 2551-2618, 2551-2619, 2551-2620, 2551-2621, 2551-2622, 2551-2623, 2551-2624, 2551-2625, 2551-2626, 2551-2627, 2551-2628, 2551-2629, 2551-2630, 2551-2631, 2553-2570, 2553-2571, 2553-2572, 2553-2573, 2553-2574, 2553-2575, 2553-2576, 2553-2577, 2553-2578, 2553-2579, 2553-2580, 2553-2581, 2553-2582, 2553-2583, 2553-2584, 2553-2585, 2553-2586, 2553-2587, 2553-2588, 2553-2589, 2553-2590, 2553-2591, 2553-2592, 2553-2593, 2553-2594, 2553-2595, 2553-2596, 2553-2597, 2553-2598, 2553-2599, 2553-2600, 2553-2601, 2553-2602, 2553-2603, 2553-2604, 2553-2605, 2553-2606, 2553-2607, 2553-2608, 2553-2609, 2553-2610, 2553-2611, 2553-2612, 2553-2613, 2553-2614, 2553-2615, 2553-2616, 2553-2617, 2553-2618, 2553-2619, 2553-2620, 2553-2621, 2553-2622, 2553-2623, 2553-2624, 2553-2625, 2553-2626, 2553-2627, 2553-2628, 2553-2629, 2553-2630, 2553-2631, 2554-2573, 2554-2574, 2554-2575, 2554-2576, 2554-2577, 2554-2578, 2554-2579, 2554-2580, 2554-2581, 2554-2582, 2554-2583, 2554-2584, 2554-2585, 2554-2586, 2554-2587, 2554-2588, 2554-2589, 2554-2590, 2554-2591, 2554-2592, 2554-2593, 2554-2594, 2554-2595, 2554-2596, 2554-2597, 2554-2598, 2554-2599, 2554-2600, 2554-2601, 2554-2602, 2554-2603, 2554-2604, 2554-2605, 2554-2606, 2554-2607, 2554-2608, 2554-2609, 2554-2610, 2554-2611, 2554-2612, 2554-2613, 2554-2614, 2554-2615, 2554-2616, 2554-2617, 2554-2618, 2554-2619, 2554-2620, 2554-2621, 2554-2622, 2554-2623, 2554-2624, 2554-2625, 2554-2626, 2554-2627, 2554-2628, 2554-2629, 2554-2630, 2554-2631, 2555-2572, 2555-2573, 2555-2574, 2555-2575, 2555-2576, 2555-2577, 2555-2578, 2555-2579, 2555-2580, 2555-2581, 2555-2582, 2555-2583, 2555-2584, 2555-2585, 2555-2586, 2555-2587, 2555-2588, 2555-2589, 2555-2590, 2555-2591, 2555-2592, 2555-2593, 2555-2594, 2555-2595, 2555-2596, 2555-2597, 2555-2598, 2555-2599, 2555-2600, 2555-2601, 2555-2602, 2555-2603, 2555-2604, 2555-2605, 2555-2606, 2555-2607, 2555-2608, 2555-2609, 2555-2610, 2555-2611, 2555-2612, 2555-2613, 2555-2614, 2555-2615, 2555-2616, 2555-2617, 2555-2618, 2555-2619, 2555-2620, 2555-2621, 2555-2622, 2555-2623, 2555-2624, 2555-2625, 2555-2626, 2555-2627, 2555-2628, 2555-2629, 2555-2630, 2555-2631, 2556-2573, 2556-2574, 2556-2575, 2556-2576, 2556-2577, 2556-2578, 2556-2579, 2556-2580, 2556-2581, 2556-2582, 2556-2583, 2556-2584, 2556-2585, 2556-2586, 2556-2587, 2556-2588, 2556-2589, 2556-2590, 2556-2591, 2556-2592, 2556-2593, 2556-2594, 2556-2595, 2556-2596, 2556-2597, 2556-2598, 2556-2599, 2556-2600, 2556-2601, 2556-2602, 2556-2603, 2556-2604, 2556-2605, 2556-2606, 2556-2607, 2556-2608, 2556-2609, 2556-2610, 2556-2611, 2556-2612, 2556-2613, 2556-2614, 2556-2615, 2556-2616, 2556-2617, 2556-2618, 2556-2619, 2556-2620, 2556-2621, 2556-2622, 2556-2623, 2556-2624, 2556-2625, 2556-2626, 2556-2627, 2556-2628, 2556-2629, 2556-2630, 2556-2631, 2557-2574, 2557-2575, 2557-2576, 2557-2577, 2557-2578, 2557-2579, 2557-2580, 2557-2581, 2557-2582, 2557-2583, 2557-2584, 2557-2585, 2557-2586, 2557-2587, 2557-2588, 2557-2589, 2557-2590, 2557-2591, 2557-2592, 2557-2593, 2557-2594, 2557-2595, 2557-2596, 2557-2597, 2557-2598, 2557-2599, 2557-2600, 2557-2601, 2557-2602, 2557-2603, 2557-2604, 2557-2605, 2557-2606, 2557-2607, 2557-2608, 2557-2609, 2557-2610, 2557-2611, 2557-2612, 2557-2613, 2557-2614, 2557-2615, 2557-2616, 2557-2617, 2557-2618, 2557-2619, 2557-2620, 2557-2621, 2557-2622, 2557-2623, 2557-2624, 2557-2625, 2557-2626, 2557-2627, 2557-2628, 2557-2629, 2557-2630, 2557-2631, 2558-2575, 2558-2576, 2558-2577, 2558-2578, 2558-2579, 2558-2580, 2558-2581, 2558-2582, 2558-2583, 2558-2584, 2558-2585, 2558-2586, 2558-2587, 2558-2588, 2558-2589, 2558-2590, 2558-2591, 2558-2592, 2558-2593, 2558-2594, 2558-2595, 2558-2596, 2558-2597, 2558-2598, 2558-2599, 2558-2600, 2558-2601, 2558-2602, 2558-2603, 2558-2604, 2558-2605, 2558-2606, 2558-2607, 2558-2608, 2558-2609, 2558-2610, 2558-2611, 2558-2612, 2558-2613, 2558-2614, 2558-2615, 2558-2616, 2558-2617, 2558-2618, 2558-2619, 2558-2620, 2558-2621, 2558-2622, 2558-2623, 2558-2624, 2558-2625, 2558-2626, 2558-2627, 2558-2628, 2558-2629, 2558-2630, 2558-2631, 2559-2576, 2559-2577, 2559-2578, 2559-2579, 2559-2580, 2559-2581, 2559-2582, 2559-2583, 2559-2584, 2559-2585, 2559-2586, 2559-2587, 2559-2588, 2559-2589, 2559-2590, 2559-2591, 2559-2592, 2559-2593, 2559-2594, 2559-2595, 2559-2596, 2559-2597, 2559-2598, 2559-2599, 2559-2600, 2559-2601, 2559-2602, 2559-2603, 2559-2604, 2559-2605, 2559-2606, 2559-2607, 2559-2608, 2559-2609, 2559-2610, 2559-2611, 2559-2612, 2559-2613, 2559-2614, 2559-2615, 2559-2616, 2559-2617, 2559-2618, 2559-2619, 2559-2620, 2559-2621, 2559-2622, 2559-2623, 2559-2624, 2559-2625, 2559-2626, 2559-2627, 2559-2628, 2559-2629, 2559-2630, 2559-2631, 2560-2577, 2560-2578, 2560-2579, 2560-2580, 2560-2581, 2560-2582, 2560-2583, 2560-2584, 2560-2585, 2560-2586, 2560-2587, 2560-2588, 2560-2589, 2560-2590, 2560-2591, 2560-2592, 2560-2593, 2560-2594, 2560-2595, 2560-2596, 2560-2597, 2560-2598, 2560-2599, 2560-2600, 2560-2601, 2560-2602, 2560-2603, 2560-2604, 2560-2605, 2560-2606, 2560-2607, 2560-2608, 2560-2609, 2560-2610, 2560-2611, 2560-2612, 2560-2613, 2560-2614, 2560-2615, 2560-2616, 2560-2617, 2560-2618, 2560-2619, 2560-2620, 2560-2621, 2560-2622, 2560-2623, 2560-2624, 2560-2625, 2560-2626, 2560-2627, 2560-2628, 2560-2629, 2560-2630, 2560-2631, 2561-2578, 2561-2579, 2561-2580, 2561-2581, 2561-2582, 2561-2583, 2561-2584, 2561-2585, 2561-2586, 2561-2587, 2561-2588, 2561-2589, 2561-2590, 2561-2591, 2561-2592, 2561-2593, 2561-2594, 2561-2595, 2561-2596, 2561-2597, 2561-2598, 2561-2599, 2561-2600, 2561-2601, 2561-2602, 2561-2603, 2561-2604, 2561-2605, 2561-2606, 2561-2607, 2561-2608, 2561-2609, 2561-2610, 2561-2611, 2561-2612, 2561-2613, 2561-2614, 2561-2615, 2561-2616, 2561-2617, 2561-2618, 2561-2619, 2561-2620, 2561-2621, 2561-2622, 2561-2623, 2561-2624, 2561-2625, 2561-2626, 2561-2627, 2561-2628, 2561-2629, 2561-2630, 2561-2631, 2562-2577, 2562-2578, 2562-2579, 2562-2580, 2562-2581, 2562-2582, 2562-2583, 2562-2584, 2562-2585, 2562-2586, 2562-2587, 2562-2588, 2562-2589, 2562-2590, 2562-2591, 2562-2592, 2562-2593, 2562-2594, 2562-2595, 2562-2596, 2562-2597, 2562-2598, 2562-2599, 2562-2600, 2562-2601, 2562-2602, 2562-2603, 2562-2604, 2562-2605, 2562-2606, 2562-2607, 2562-2608, 2562-2609, 2562-2610, 2562-2611, 2562-2612, 2562-2613, 2562-2614, 2562-2615, 2562-2616, 2562-2617, 2562-2618, 2562-2619, 2562-2620, 2562-2621, 2562-2622, 2562-2623, 2562-2624, 2562-2625, 2562-2626, 2562-2627, 2562-2628, 2562-2629, 2562-2630, 2562-2631, 2563-2580, 2563-2581, 2563-2582, 2563-2583, 2563-2584, 2563-2585, 2563-2586, 2563-2587, 2563-2588, 2563-2589, 2563-2590, 2563-2591, 2563-2592, 2563-2593, 2563-2594, 2563-2595, 2563-2596, 2563-2597, 2563-2598, 2563-2599, 2563-2600, 2563-2601, 2563-2602, 2563-2603, 2563-2604, 2563-2605, 2563-2606, 2563-2607, 2563-2608, 2563-2609, 2563-2610, 2563-2611, 2563-2612, 2563-2613, 2563-2614, 2563-2615, 2563-2616, 2563-2617, 2563-2618, 2563-2619, 2563-2620, 2563-2621, 2563-2622, 2563-2623, 2563-2624, 2563-2625, 2563-2626, 2563-2627, 2563-2628, 2563-2629, 2563-2630, 2563-2631, 2564-2581, 2564-2582, 2564-2583, 2564-2584, 2564-2585, 2564-2586, 2564-2587, 2564-2588, 2564-2589, 2564-2590, 2564-2591, 2564-2592, 2564-2593, 2564-2594, 2564-2595, 2564-2596, 2564-2597, 2564-2598, 2564-2599, 2564-2600, 2564-2601, 2564-2602, 2564-2603, 2564-2604, 2564-2605, 2564-2606, 2564-2607, 2564-2608, 2564-2609, 2564-2610, 2564-2611, 2564-2612, 2564-2613, 2564-2614, 2564-2615, 2564-2616, 2564-2617, 2564-2618, 2564-2619, 2564-2620, 2564-2621, 2564-2622, 2564-2623, 2564-2624, 2564-2625, 2564-2626, 2564-2627, 2564-2628, 2564-2629, 2564-2630, 2564-2631, 2565-2584, 2565-2585, 2565-2586, 2565-2587, 2565-2588, 2565-2589, 2565-2590, 2565-2591, 2565-2592, 2565-2593, 2565-2594, 2565-2595, 2565-2596, 2565-2597, 2565-2598, 2565-2599, 2565-2600, 2565-2601, 2565-2602, 2565-2603, 2565-2604, 2565-2605, 2565-2606, 2565-2607, 2565-2608, 2565-2609, 2565-2610, 2565-2611, 2565-2612, 2565-2613, 2565-2614, 2565-2615, 2565-2616, 2565-2617, 2565-2618, 2565-2619, 2565-2620, 2565-2621, 2565-2622, 2565-2623, 2565-2624, 2565-2625, 2565-2626, 2565-2627, 2565-2628, 2565-2629, 2565-2630, 2565-2631, 2566-2583, 2566-2584, 2566-2585, 2566-2586, 2566-2587, 2566-2588, 2566-2589, 2566-2590, 2566-2591, 2566-2592, 2566-2593, 2566-2594, 2566-2595, 2566-2596, 2566-2597, 2566-2598, 2566-2599, 2566-2600, 2566-2601, 2566-2602, 2566-2603, 2566-2604, 2566-2605, 2566-2606, 2566-2607, 2566-2608, 2566-2609, 2566-2610, 2566-2611, 2566-2612, 2566-2613, 2566-2614, 2566-2615, 2566-2616, 2566-2617, 2566-2618, 2566-2619, 2566-2620, 2566-2621, 2566-2622, 2566-2623, 2566-2624, 2566-2625, 2566-2626, 2566-2627, 2566-2628, 2566-2629, 2566-2630, 2566-2631, 2567-2584, 2567-2585, 2567-2586, 2567-2587, 2567-2588, 2567-2589, 2567-2590, 2567-2591, 2567-2592, 2567-2593, 2567-2594, 2567-2595, 2567-2596, 2567-2597, 2567-2598, 2567-2599, 2567-2600, 2567-2601, 2567-2602, 2567-2603, 2567-2604, 2567-2605, 2567-2606, 2567-2607, 2567-2608, 2567-2609, 2567-2610, 2567-2611, 2567-2612, 2567-2613, 2567-2614, 2567-2615, 2567-2616, 2567-2617, 2567-2618, 2567-2619, 2567-2620, 2567-2621, 2567-2622, 2567-2623, 2567-2624, 2567-2625, 2567-2626, 2567-2627, 2567-2628, 2567-2629, 2567-2630, 2567-2631, 2568-2585, 2568-2586, 2568-2587, 2568-2588, 2568-2589, 2568-2590, 2568-2591, 2568-2592, 2568-2593, 2568-2594, 2568-2595, 2568-2596, 2568-2597, 2568-2598, 2568-2599, 2568-2600, 2568-2601, 2568-2602, 2568-2603, 2568-2604, 2568-2605, 2568-2606, 2568-2607, 2568-2608, 2568-2609, 2568-2610, 2568-2611, 2568-2612, 2568-2613, 2568-2614, 2568-2615, 2568-2616, 2568-2617, 2568-2618, 2568-2619, 2568-2620, 2568-2621, 2568-2622, 2568-2623, 2568-2624, 2568-2625, 2568-2626, 2568-2627, 2568-2628, 2568-2629, 2568-2630, 2568-2631, 2569-2586, 2569-2587, 2569-2588, 2569-2589, 2569-2590, 2569-2591, 2569-2592, 2569-2593, 2569-2594, 2569-2595, 2569-2596, 2569-2597, 2569-2598, 2569-2599, 2569-2600, 2569-2601, 2569-2602, 2569-2603, 2569-2604, 2569-2605, 2569-2606, 2569-2607, 2569-2608, 2569-2609, 2569-2610, 2569-2611, 2569-2612, 2569-2613, 2569-2614, 2569-2615, 2569-2616, 2569-2617, 2569-2618, 2569-2619, 2569-2620, 2569-2621, 2569-2622, 2569-2623, 2569-2624, 2569-2625, 2569-2626, 2569-2627, 2569-2628, 2569-2629, 2569-2630, 2569-2631, 2569-2586, 2569-2587, 2569-2588, 2569-2589, 2569-2590, 2569-2591, 2569-2592, 2569-2593, 2569-2594, 2569-2595, 2569-2596, 2569-2597, 2569-2598, 2569-2599, 2569-2600, 2569-2601, 2569-2602, 2569-2603, 2569-2604, 2569-2605, 2569-2606, 2569-2607, 2569-2608, 2569-2609, 2569-2610, 2569-2611, 2569-2612, 2569-2613, 2569-2614, 2569-2615, 2569-2616, 2569-2617, 2569-2618, 2569-2619, 2569-2620, 2569-2621, 2569-2622, 2569-2623, 2569-2624, 2569-2625, 2569-2626, 2569-2627, 2569-2628, 2569-2629, 2569-2630, 2569-2631, 2571-2588, 2571-2589, 2571-2590, 2571-2591, 2571-2592, 2571-2593, 2571-2594, 2571-2595, 2571-2596, 2571-2597, 2571-2598, 2571-2599, 2571-2600, 2571-2601, 2571-2602, 2571-2603, 2571-2604, 2571-2605, 2571-2606, 2571-2607, 2571-2608, 2571-2609, 2571-2610, 2571-2611, 2571-2612, 2571-2613, 2571-2614, 2571-2615, 2571-2616, 2571-2617, 2571-2618, 2571-2619, 2571-2620, 2571-2621, 2571-2622, 2571-2623, 2571-2624, 2571-2625, 2571-2626, 2571-2627, 2571-2628, 2571-2629, 2571-2630, 2571-2631, 2572-2589, 2572-2590, 2572-2591, 2572-2592, 2572-2593, 2572-2594, 2572-2595, 2572-2596, 2572-2597, 2572-2598, 2572-2599, 2572-2600, 2572-2601, 2572-2602, 2572-2603, 2572-2604, 2572-2605, 2572-2606, 2572-2607, 2572-2608, 2572-2609, 2572-2610, 2572-2611, 2572-2612, 2572-2613, 2572-2614, 2572-2615, 2572-2616, 2572-2617, 2572-2618, 2572-2619, 2572-2620, 2572-2621, 2572-2622, 2572-2623, 2572-2624, 2572-2625, 2572-2626, 2572-2627, 2572-2628, 2572-2629, 2572-2630, 2572-2631, 2573-2590, 2573-2591, 2573-2592, 2573-2593, 2573-2594, 2573-2595, 2573-2596, 2573-2597, 2573-2598, 2573-2599, 2573-2600, 2573-2601, 2573-2602, 2573-2603, 2573-2604, 2573-2605, 2573-2606, 2573-2607, 2573-2608, 2573-2609, 2573-2610, 2573-2611, 2573-2612, 2573-2613, 2573-2614, 2573-2615, 2573-2616, 2573-2617, 2573-2618, 2573-2619, 2573-2620, 2573-2621, 2573-2622, 2573-2623, 2573-2624, 2573-2625, 2573-2626, 2573-2627, 2573-2628, 2573-2629, 2573-2630, 2573-2631, 2574-2591, 2574-2592, 2574-2593, 2574-2594, 2574-2595, 2574-2596, 2574-2597, 2574-2598, 2574-2599, 2574-2600, 2574-2601, 2574-2602, 2574-2603, 2574-2604, 2574-2605, 2574-2606, 2574-2607, 2574-2608, 2574-2609, 2574-2610, 2574-2611, 2574-2612, 2574-2613, 2574-2614, 2574-2615, 2574-2616, 2574-2617, 2574-2618, 2574-2619, 2574-2620, 2574-2621, 2574-2622, 2574-2623, 2574-2624, 2574-2625, 2574-2626, 2574-2627, 2574-2628, 2574-2629, 2574-2630, 2574-2631, 2575-2592, 2575-2593, 2575-2594, 2575-2595, 2575-2596, 2575-2597, 2575-2598, 2575-2599, 2575-2600, 2575-2601, 2575-2602, 2575-2603, 2575-2604, 2575-2605, 2575-2606, 2575-2607, 2575-2608, 2575-2609, 2575-2610, 2575-2611, 2575-2612, 2575-2613, 2575-2614, 2575-2615, 2575-2616, 2575-2617, 2575-2618, 2575-2619, 2575-2620, 2575-2621, 2575-2622, 2575-2623, 2575-2624, 2575-2625, 2575-2626, 2575-2627, 2575-2628, 2575-2629, 2575-2630, 2575-2631, 2576-2593, 2576-2594, 2576-2595, 2576-2596, 2576-2597, 2576-2598, 2576-2599, 2576-2600, 2576-2601, 2576-2602, 2576-2603, 2576-2604, 2576-2605, 2576-2606, 2576-2607, 2576-2608, 2576-2609, 2576-2610, 2576-2611, 2576-2612, 2576-2613, 2576-2614, 2576-2615, 2576-2616, 2576-2617, 2576-2618, 2576-2619, 2576-2620, 2576-2621, 2576-2622, 2576-2623, 2576-2624, 2576-2625, 2576-2626, 2576-2627, 2576-2628, 2576-2629, 2576-2630, 2576-2631, 2577-2594, 2577-2595, 2577-2596, 2577-2597, 2577-2598, 2577-2599, 2577-2600, 2577-2601, 2577-2602, 2577-2603, 2577-2604, 2577-2605, 2577-2606, 2577-2607, 2577-2608, 2577-2609, 2577-2610, 2577-2611, 2577-2612, 2577-2613, 2577-2614, 2577-2615, 2577-2616, 2577-2617, 2577-2618, 2577-2619, 2577-2620, 2577-2621, 2577-2622, 2577-2623, 2577-2624, 2577-2625, 2577-2626, 2577-2627, 2577-2628, 2577-2629, 2577-2630, 2577-2631, 2578-2597, 2578-2598, 2578-2599, 2578-2600, 2578-2601, 2578-2602, 2578-2603, 2578-2604, 2578-2605, 2578-2606, 2578-2607, 2578-2608, 2578-2609, 2578-2610, 2578-2611, 2578-2612, 2578-2613, 2578-2614, 2578-2615, 2578-2616, 2578-2617, 2578-2618, 2578-2619, 2578-2620, 2578-2621, 2578-2622, 2578-2623, 2578-2624, 2578-2625, 2578-2626, 2578-2627, 2578-2628, 2578-2629, 2578-2630, 2578-2631, 2579-2598, 2579-2599, 2579-2600, 2579-2601, 2579-2602, 2579-2603, 2579-2604, 2579-2605, 2579-2606, 2579-2607, 2579-2608, 2579-2609, 2579-2610, 2579-2611, 2579-2612, 2579-2613, 2579-2614, 2579-2615, 2579-2616, 2579-2617, 2579-2618, 2579-2619, 2579-2620, 2579-2621, 2579-2622, 2579-2623, 2579-2624, 2579-2625, 2579-2626, 2579-2627, 2579-2628, 2579-2629, 2579-2630, 2579-2631, 2580-2598, 2580-2599, 2580-2600, 2580-2601, 2580-2602, 2580-2603, 2580-2604, 2580-2605, 2580-2606, 2580-2607, 2580-2608, 2580-2609, 2580-2610, 2580-2611, 2580-2612, 2580-2613, 2580-2614, 2580-2615, 2580-2616, 2580-2617, 2580-2618, 2580-2619, 2580-2620, 2580-2621, 2580-2622, 2580-2623, 2580-2624, 2580-2625, 2580-2626, 2580-2627, 2580-2628, 2580-2629, 2580-2630, 2580-2631, 2581-2597, 2581-2598, 2581-2599, 2581-2600, 2581-2601, 2581-2602, 2581-2603, 2581-2604, 2581-2605, 2581-2606, 2581-2607, 2581-2608, 2581-2609, 2581-2610, 2581-2611, 2581-2612, 2581-2613, 2581-2614, 2581-2615, 2581-2616, 2581-2617, 2581-2618, 2581-2619, 2581-2620, 2581-2621, 2581-2622, 2581-2623, 2581-2624, 2581-2625, 2581-2626, 2581-2627, 2581-2628, 2581-2629, 2581-2630, 2581-2631, 2582-2600, 2582-2601, 2582-2602, 2582-2603, 2582-2604, 2582-2605, 2582-2606, 2582-2607, 2582-2608, 2582-2609, 2582-2610, 2582-2611, 2582-2612, 2582-2613, 2582-2614, 2582-2615, 2582-2616, 2582-2617, 2582-2618, 2582-2619, 2582-2620, 2582-2621, 2582-2622, 2582-2623, 2582-2624, 2582-2625, 2582-2626, 2582-2627, 2582-2628, 2582-2629, 2582-2630, 2582-2631, 2583-2601, 2583-2602, 2583-2603, 2583-2604, 2583-2605, 2583-2606, 2583-2607, 2583-2608, 2583-2609, 2583-2610, 2583-2611, 2583-2612, 2583-2613, 2583-2614, 2583-2615, 2583-2616, 2583-2617, 2583-2618, 2583-2619, 2583-2620, 2583-2621, 2583-2622, 2583-2623, 2583-2624, 2583-2625, 2583-2626, 2583-2627, 2583-2628, 2583-2629, 2583-2630, 2583-2631, 2585-2603, 2585-2604, 2585-2605, 2585-2606, 2585-2607, 2585-2608, 2585-2609, 2585-2610, 2585-2611, 2585-2612, 2585-2613, 2585-2614, 2585-2615, 2585-2616, 2585-2617, 2585-2618, 2585-2619, 2585-2620, 2585-2621, 2585-2622, 2585-2623, 2585-2624, 2585-2625, 2585-2626, 2585-2627, 2585-2628, 2585-2629, 2585-2630, 2585-2631, 2586-2604, 2586-2605, 2586-2606, 2586-2607, 2586-2608, 2586-2609, 2586-2610, 2586-2611, 2586-2612, 2586-2613, 2586-2614, 2586-2615, 2586-2616, 2586-2617, 2586-2618, 2586-2619, 2586-2620, 2586-2621, 2586-2622, 2586-2623, 2586-2624, 2586-2625, 2586-2626, 2586-2627, 2586-2628, 2586-2629, 2586-2630, 2586-2631, 2587-2605, 2587-2606, 2587-2607, 2587-2608, 2587-2609, 2587-2610, 2587-2611, 2587-2612, 2587-2613, 2587-2614, 2587-2615, 2587-2616, 2587-2617, 2587-2618, 2587-2619, 2587-2620, 2587-2621, 2587-2622, 2587-2623, 2587-2624, 2587-2625, 2587-2626, 2587-2627, 2587-2628, 2587-2629, 2587-2630, 2587-2631, 2588-2606, 2588-2607, 2588-2608, 2588-2609, 2588-2610, 2588-2611, 2588-2612, 2588-2613, 2588-2614, 2588-2615, 2588-2616, 2588-2617, 2588-2618, 2588-2619, 2588-2620, 2588-2621, 2588-2622, 2588-2623, 2588-2624, 2588-2625, 2588-2626, 2588-2627, 2588-2628, 2588-2629, 2588-2630, 2588-2631, 2589-2607, 2589-2608, 2589-2609, 2589-2610, 2589-2611, 2589-2612, 2589-2613, 2589-2614, 2589-2615, 2589-2616, 2589-2617, 2589-2618, 2589-2619, 2589-2620, 2589-2621, 2589-2622, 2589-2623, 2589-2624, 2589-2625, 2589-2626, 2589-2627, 2589-2628, 2589-2629, 2589-2630, 2589-2631, 2590-2606, 2590-2607, 2590-2608, 2590-2609, 2590-2610, 2590-2611, 2590-2612, 2590-2613, 2590-2614, 2590-2615, 2590-2616, 2590-2617, 2590-2618, 2590-2619, 2590-2620, 2590-2621, 2590-2622, 2590-2623, 2590-2624, 2590-2625, 2590-2626, 2590-2627, 2590-2628, 2590-2629, 2590-2630, 2590-2631, 2591-2610, 2591-2611, 2591-2612, 2591-2613, 2591-2614, 2591-2615, 2591-2616, 2591-2617, 2591-2618, 2591-2619, 2591-2620, 2591-2621, 2591-2622, 2591-2623, 2591-2624, 2591-2625, 2591-2626, 2591-2627, 2591-2628, 2591-2629, 2591-2630, 2591-2631, 2592-2611, 2592-2612, 2592-2613, 2592-2614, 2592-2615, 2592-2616, 2592-2617, 2592-2618, 2592-2619, 2592-2620, 2592-2621, 2592-2622, 2592-2623, 2592-2624, 2592-2625, 2592-2626, 2592-2627, 2592-2628, 2592-2629, 2592-2630, 2592-2631, 2593-2608, 2593-2612, 2593-2613, 2593-2614, 2593-2615, 2593-2616, 2593-2617, 2593-2618, 2593-2619, 2593-2620, 2593-2621, 2593-2622, 2593-2623, 2593-2624, 2593-2625, 2593-2626, 2593-2627, 2593-2628, 2593-2629, 2593-2630, 2593-2631, 2594-2612, 2594-2613, 2594-2614, 2594-2615, 2594-2616, 2594-2617, 2594-2618, 2594-2619, 2594-2620, 2594-2621, 2594-2622, 2594-2623, 2594-2624, 2594-2625, 2594-2626, 2594-2627, 2594-2628, 2594-2629, 2594-2630, 2594-2631, 2595-2611, 2595-2612, 2595-2613, 2595-2614, 2595-2615, 2595-2616, 2595-2617, 2595-2618, 2595-2619, 2595-2620, 2595-2621, 2595-2622, 2595-2623, 2595-2624, 2595-2625, 2595-2626, 2595-2627, 2595-2628, 2595-2629, 2595-2630, 2595-2631, 2596-2614, 2596-2615, 2596-2616, 2596-2617, 2596-2618, 2596-2619, 2596-2620, 2596-2621, 2596-2622, 2596-2623, 2596-2624, 2596-2625, 2596-2626, 2596-2627, 2596-2628, 2596-2629, 2596-2630, 2596-2631, 2597-2612, 2597-2613, 2597-2614, 2597-2615, 2597-2616, 2597-2617, 2597-2618, 2597-2619, 2597-2620, 2597-2621, 2597-2622, 2597-2623, 2597-2624, 2597-2625, 2597-2626, 2597-2627, 2597-2628, 2597-2629, 2597-2630, 2597-2631, 2598-2613, 2598-2614, 2598-2615, 2598-2616, 2598-2617, 2598-2618, 2598-2619, 2598-2620, 2598-2621, 2598-2622, 2598-2623, 2598-2624, 2598-2625, 2598-2626, 2598-2627, 2598-2628, 2598-2629, 2598-2630, 2598-2631, 2599-2614, 2599-2615, 2599-2616, 2599-2617, 2599-2618, 2599-2619, 2599-2620, 2599-2621, 2599-2622, 2599-2623, 2599-2624, 2599-2625, 2599-2626, 2599-2627, 2599-2628, 2599-2629, 2599-2630, 2599-2631, 2600-2615, 2600-2616, 2600-2617, 2600-2618, 2600-2619, 2600-2620, 2600-2621, 2600-2622, 2600-2623, 2600-2624, 2600-2625, 2600-2626, 2600-2627, 2600-2628, 2600-2629, 2600-2630, 2600-2631, 2601-2616, 2601-2617, 2601-2618, 2601-2619, 2601-2620, 2601-2621, 2601-2622, 2601-2623, 2601-2624, 2601-2625, 2601-2626, 2601-2627, 2601-2628, 2601-2629, 2601-2630, 2601-2631, 2602-2618, 2602-2619, 2602-2620, 2602-2621, 2602-2622, 2602-2623, 2602-2624, 2602-2625, 2602-2626, 2602-2627, 2602-2628, 2602-2629, 2602-2630, 2602-2631, 2603-2620, 2603-2621, 2603-2622, 2603-2623, 2603-2624, 2603-2625, 2603-2626, 2603-2627, 2603-2628, 2603-2629, 2603-2630, 2603-2631, 2604-2619, 2604-2620, 2604-2621, 2604-2622, 2604-2623, 2604-2624, 2604-2625, 2604-2626, 2604-2627, 2604-2628, 2604-2629, 2604-2630, 2604-2631, 2605-2620, 2605-2621, 2605-2622, 2605-2623, 2605-2624, 2605-2625, 2605-2626, 2605-2627, 2605-2628, 2605-2629, 2605-2630, 2605-2631, 2606-2621, 2606-2622, 2606-2623, 2606-2624, 2606-2625, 2606-2626, 2606-2627, 2606-2628, 2606-2629, 2606-2630, 2606-2631, 2607-2622, 2607-2623, 2607-2624, 2607-2625, 2607-2626, 2607-2627, 2607-2628, 2607-2629, 2607-2630, 2607-2631, 2608-2623, 2608-2624, 2608-2625, 2608-2626, 2608-2627, 2608-2628, 2608-2629, 2608-2630, 2608-2631, 2609-2624, 2609-2625, 2609-2626, 2609-2627, 2609-2628, 2609-2629, 2609-2630, 2609-2631, 2610-2625, 2610-2626, 2610-2627, 2610-2628, 2610-2629, 2610-2630, 2610-2631, 2611-2626, 2611-2627, 2611-2628, 2611-2629, 2611-2630, 2611-2631, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, or 2616-2631. In certain aspects, antisense compounds or oligonucleotides target at least an 8, 9, 10, 11, 12, 13, 14, 15, or 16 contiguous nucleobases within the aforementioned nucleobase regions.

In certain embodiments, the following nucleotide regions of SEQ ID NO: 1, when targeted by antisense compounds or oligonucleotides, display at least 50% inhibition: 30-49, 48-63, 150-169, 151-170, 152-171, 154-169, 154-173, 156-171, 156-175, 157-176, 158-173, 158-177, 480-499, 600-619, 638-657, 644-663, 738-757, 1089-1108, 1135-1154, 1141-1160, 1147-1166, 1150-1169, 1153-1172, 1159-1178, 1162-1181, 1165-1184, 1171-1186, 1171-1190, 1173-1188, 1173-1192, 1175-1190, 1175-1194, 1177-1196, 1183-1202, 1208-1227, 1235-1254, 1298-1317, 1304-1323, 1310-1329, 1316-1335, 1319-1338, 1322-1341, 1328-1347, 1349-1368, 1355-1374, 1393-1412, 1396-1415, 1399-1418, 1405-1424, 1421-1440, 1621-1640, 1646-1665, 1646-1665, 1647-1666, 1689-1708, 1749-1768, 1763-1782, 1912-1931, 2073-2092, 2085-2104, 2166-2185, 2172-2191, 2189-2208, 2191-2210, 2193-2212, 2195-2210, 2195-2214, 2196-2215, 2197-2212, 2197-2216, 2202-2221, 2223-2238, 2223-2242, 2225-2240, 2226-2245, 2227-2242, 2227-2246, 2238-2257, 2241-2260, 2267-2286, 2361-2380, 2388-2407, 2397-2416, 2448-2467, 2453-2472, 2455-2474, 2457-2472, 2457-2476, 2459-2474, 2459-2478, 2461-2476, 2461-2480, 2532-2551, 2550-2569, 2551-2566, 2551-2570, 2552-2568, 2552-2570, 2552-2571, 2553-2568, 2553-2570, 2553-2571, 2553-2572, 2554-2571, 2554-2572, 2554-2573, 2555-2570, 2555-2572, 2555-2574, 2556-2573, 2556-2574, 2556-2575, 2557-2573, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2576, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2585, 2570-2587, 2570-2589, 2571-2586, 2571-2588, 2571-2590, 2572-2589, 2572-2590, 2572-2591, 2573-2590, 2573-2592, 2574-2590, 2574-2591, 2574-2593, 2575-2590, 2575-2591, 2575-2592, 2575-2594, 2576-2593, 2576-2595, 2577-2594, 2577-2595, 2577-2596, 2578-2594, 2578-2596, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2601, 2584-2602, 2584-2603, 2585-2601, 2585-2603, 2585-2604, 2586-2601, 2586-2602, 2586-2604, 2586-2605, 2587-2602, 2587-2603, 2587-2605, 2587-2606, 2588-2603, 2588-2604, 2588-2605, 2588-2606, 2588-2607, 2589-2604, 2589-2605, 2589-2606, 2589-2607, 2589-2608, 2590-2605, 2590-2606, 2590-2607, 2590-2608, 2590-2609, 2590-2609, 2591-2607, 2591-2608, 2591-2609, 2591-2610, 2592-2607, 2592-2608, 2592-2609, 2592-2610, 2592-2611, 2593-2608, 2593-2609, 2593-2610, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2631, and 2616-2631.

In certain embodiments, the following nucleotide regions of SEQ ID NO: 2, when targeted by antisense compounds or oligonucleotides, display at least 50% inhibition: 1608-1627, 1685-1704, 1686-1705, 1751-1770, 1769-1784, 1871-1890, 1872-1891, 1873-1892, 1875-1890, 1875-1894, 1877-1892, 1877-1896, 1878-1897, 1879-1894, 1879-1898, 2288-2307, 2808-2827, 2846-2865, 2852-2871, 2946-2965, 3773-3792, 3819-3838, 3825-3844, 3831-3850, 3834-3853, 3837-3856, 3843-3862, 4151-4166, 4151-4170, 4153-4172, 4159-4178, 4184-4203, 4211-4230, 4609-4628, 4612-4631, 4615-4634, 4621-4640, 4642-4661, 4648-4667, 4686-4705, 4689-4708, 4692-4711, 4698-4717, 4714-4733, 5270-5289, 5295-5314, 5296-5315, 5830-5849, 5890-5909, 5904-5923, 6406-6425, 6662-6681, 6674-6693, 6954-6973, 6960-6979, 6977-6996, 6979-6998, 6981-7000, 6983-6998, 6983-7002, 6984-7003, 6985-7000, 6985-7004, 6990-7009, 7122-7141, 7125-7144, 7151-7170, 7353-7372, 7362-7381, 7683-7702, 7688-7707, 7690-7709, 7692-7707, 7692-7711, 7694-7709, 7694-7713, 7696-7711, 7696-7715, 7767-7786, 7785-7804, 7786-7801, 7787-7803, 7787-7805, 7787-7806, 7788-7803, 7788-7805, 7788-7806, 7788-7807, 7789-7806, 7789-7807, 7789-7808, 7790-7805, 7790-7807, 7790-7809, 7791-7808, 7791-7809, 7791-7810, 7792-7808, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7811, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7820, 7805-7822, 7805-7824, 7806-7821, 7806-7823, 7806-7825, 7807-7824, 7807-7825, 7807-7826, 7808-7825, 7808-7827, 7809-7825, 7809-7826, 7809-7828, 7810-7825, 7810-7826, 7810-7827, 7810-7829, 7811-7828, 7811-7830, 7812-7829, 7812-7830, 7812-7831, 7813-7829, 7813-7831, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7836, 7819-7837, 7819-7838, 7820-7836, 7820-7838, 7820-7839, 7821-7836, 7821-7837, 7821-7839, 7821-7840, 7822-7837, 7822-7838, 7822-7840, 7822-7841, 7823-7838, 7823-7839, 7823-7839, 7823-7840, 7823-7841, 7823-7842, 7824-7839, 7824-7840, 7824-7840, 7824-7841, 7824-7842, 7824-7843, 7825-7840, 7825-7841, 7825-7842, 7825-7843, 7825-7844, 7826-7842, 7826-7843, 7826-7844, 7826-7845, 7827-7842, 7827-7843, 7827-7844, 7827-7845, 7827-7846, 7828-7843, 7828-7844, 7828-7845, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, and 7846-7862.

In certain embodiments, the following nucleotide regions of SEQ ID NO: 1, when targeted by antisense compounds or oligonucleotides, display at least 60% inhibition: 48-63, 150-169, 152-171, 154-169, 154-173, 156-171, 156-175, 158-173, 158-177, 600-619, 1135-1154, 1141-1160, 1147-1166, 1153-1172, 1171-1186, 1173-1188, 1175-1190, 1749-1768, 1763-1782, 1763-1782, 1912-1931, 2189-2208, 2191-2210, 2193-2212, 2195-2210, 2195-2214, 2197-2212, 2197-2216, 2223-2238, 2225-2240, 2227-2242, 2238-2257, 2448-2467, 2453-2472, 2455-2474, 2457-2472, 2457-2476, 2459-2474, 2459-2478, 2461-2476, 2461-2480, 2550-2569, 2551-2566, 2552-2571, 2553-2568, 2553-2570, 2553-2571, 2553-2572, 2554-2571, 2554-2572, 2554-2573, 2555-2572, 2555-2574, 2556-2573, 2556-2574, 2556-2575, 2557-2574, 2557-2575, 2557-2576, 2558-2575, 2558-2576, 2558-2577, 2559-2576, 2559-2577, 2559-2578, 2560-2577, 2560-2578, 2560-2579, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2583, 2568-2585, 2568-2587, 2569-2586, 2569-2588, 2570-2587, 2570-2589, 2571-2588, 2572-2590, 2572-2591, 2573-2590, 2573-2592, 2574-2591, 2574-2593, 2575-2590, 2575-2592, 2575-2594, 2576-2593, 2576-2595, 2577-2594, 2577-2595, 2577-2596, 2578-2594, 2578-2597, 2579-2598, 2580-2596, 2580-2597, 2580-2598, 2580-2599, 2581-2597, 2581-2598, 2581-2599, 2581-2600, 2582-2598, 2582-2599, 2582-2600, 2582-2601, 2583-2599, 2583-2600, 2583-2601, 2583-2602, 2584-2600, 2584-2602, 2584-2603, 2585-2601, 2585-2603, 2585-2604, 2586-2602, 2586-2604, 2586-2605, 2587-2603, 2587-2605, 2587-2606, 2588-2603, 2588-2604, 2588-2606, 2588-2607, 2589-2605, 2589-2606, 2589-2607, 2589-2608, 2590-2605, 2590-2606, 2590-2607, 2590-2608, 2590-2609, 2591-2607, 2591-2609, 2591-2610, 2592-2608, 2592-2609, 2592-2611, 2593-2608, 2593-2609, 2593-2612, 2594-2609, 2594-2610, 2594-2611, 2594-2612, 2594-2613, 2595-2610, 2595-2611, 2595-2612, 2595-2613, 2595-2614, 2596-2611, 2596-2612, 2596-2613, 2596-2614, 2596-2615, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2618, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2626, 2611-2627, 2611-2628, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2630, 2615-2631, 2615-2631, and 2616-2631.

In certain embodiments, the following nucleotide regions of SEQ ID NO: 2, when targeted by antisense compounds or oligonucleotides, display at least 60% inhibition: 1685-1704, 1686-1705, 1769-1784, 1871-1890, 1873-1892, 1875-1890, 1875-1894, 1877-1892, 1877-1896, 1879-1894, 1879-1898, 2808-2827, 3819-3838, 3825-3844, 3831-3850, 3837-3856, 4151-4166, 5890-5909, 5904-5923, 5904-5923, 6406-6425, 6977-6996, 6979-6998, 6981-7000, 6983-6998, 6983-7002, 6985-7000, 6985-7004, 7122-7141, 7683-7702, 7688-7707, 7690-7709, 7692-7707, 7692-7711, 7694-7709, 7696-7711, 7696-7715, 7786-7801, 7787-7806, 7788-7803, 7788-7805, 7788-7806, 7788-7807, 7789-7806, 7789-7807, 7789-7808, 7790-7807, 7790-7809, 7791-7808, 7791-7809, 7791-7810, 7792-7809, 7792-7810, 7792-7811, 7793-7810, 7793-7811, 7793-7812, 7794-7811, 7794-7812, 7794-7813, 7795-7812, 7795-7813, 7795-7814, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7818, 7803-7820, 7803-7822, 7804-7821, 7804-7823, 7805-7822, 7805-7824, 7806-7823, 7806-7825, 7807-7824, 7807-7825, 7807-7826, 7808-7825, 7808-7827, 7809-7826, 7809-7828, 7810-7825, 7810-7827, 7810-7829, 7811-7828, 7811-7830, 7812-7829, 7812-7830, 7812-7831, 7813-7829, 7813-7832, 7814-7833, 7815-7831, 7815-7832, 7815-7833, 7815-7834, 7816-7832, 7816-7833, 7816-7834, 7816-7835, 7817-7833, 7817-7834, 7817-7835, 7817-7836, 7818-7834, 7818-7835, 7818-7836, 7818-7837, 7819-7835, 7819-7837, 7819-7838, 7820-7836, 7820-7838, 7820-7839, 7821-7837, 7821-7839, 7821-7840, 7822-7838, 7822-7840, 7822-7841, 7823-7838, 7823-7839, 7823-7841, 7823-7842, 7824-7840, 7824-7841, 7824-7842, 7824-7843, 7825-7840, 7825-7841, 7825-7842, 7825-7843, 7825-7844, 7826-7842, 7826-7844, 7826-7845, 7827-7843, 7827-7844, 7827-7846, 7828-7843, 7828-7844, 7828-7847, 7829-7844, 7829-7845, 7829-7846, 7829-7847, 7829-7848, 7830-7845, 7830-7846, 7830-7847, 7830-7848, 7830-7849, 7831-7846, 7831-7847, 7831-7848, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7853, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, 7846-7862, and 7847-7862.

In certain embodiments, the following nucleotide regions of SEQ ID NO: 1, when targeted by antisense compounds or oligonucleotides, display at least 70% inhibition: 48-63, 150-169, 152-171, 154-169, 154-173, 156-171, 156-175, 158-173, 158-177, 1135-1154, 1141-1160, 1147-1166, 1171-1186, 1173-1188, 1175-1190, 1749-1768, 1763-1782, 1912-1931, 2193-2212, 2195-2210, 2195-2214, 2197-2212, 2197-2216, 2223-2238, 2225-2240, 2227-2242, 2453-2472, 2455-2474, 2457-2472, 2457-2476, 2459-2474, 2461-2476, 2461-2480, 2550-2569, 2551-2566, 2552-2571, 2553-2570, 2553-2571, 2553-2572, 2554-2571, 2554-2572, 2554-2573, 2554-2573, 2555-2572, 2555-2574, 2555-2574, 2556-2573, 2556-2574, 2556-2575, 2557-2574, 2557-2576, 2558-2575, 2558-2576, 2558-2577, 2559-2576, 2559-2577, 2559-2578, 2560-2577, 2560-2578, 2560-2579, 2561-2578, 2561-2579, 2561-2580, 2562-2577, 2562-2579, 2562-2581, 2563-2578, 2563-2580, 2563-2582, 2564-2581, 2564-2583, 2565-2584, 2566-2583, 2566-2585, 2567-2582, 2567-2584, 2567-2586, 2568-2585, 2568-2587, 2569-2586, 2569-2588, 2570-2587, 2570-2589, 2571-2588, 2571-2590, 2572-2589, 2572-2591, 2573-2590, 2573-2592, 2574-2591, 2574-2593, 2575-2592, 2575-2594, 2576-2593, 2576-2595, 2577-2594, 2577-2596, 2578-2597, 2579-2598, 2580-2596, 2580-2598, 2580-2599, 2581-2597, 2581-2600, 2582-2598, 2582-2600, 2582-2601, 2583-2599, 2583-2601, 2583-2602, 2584-2600, 2584-2602, 2584-2603, 2585-2601, 2585-2603, 2585-2604, 2586-2605, 2587-2606, 2588-2604, 2588-2606, 2588-2607, 2589-2605, 2589-2606, 2589-2607, 2589-2608, 2590-2605, 2590-2606, 2590-2607, 2590-2609, 2591-2607, 2591-2610, 2592-2611, 2593-2608, 2593-2612, 2594-2609, 2594-2610, 2594-2612, 2594-2613, 2595-2610, 2595-2611, 2595-2612, 2595-2613, 2595-2614, 2596-2611, 2596-2614, 2596-2615, 2597-2612, 2597-2613, 2597-2614, 2597-2615, 2597-2616, 2598-2613, 2598-2614, 2598-2615, 2598-2616, 2598-2617, 2599-2614, 2599-2615, 2599-2616, 2599-2617, 2599-2618, 2600-2615, 2600-2616, 2600-2617, 2600-2618, 2600-2619, 2601-2616, 2601-2617, 2601-2618, 2601-2619, 2601-2620, 2602-2617, 2602-2618, 2602-2619, 2602-2620, 2602-2621, 2603-2619, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2626, 2608-2627, 2609-2624, 2609-2625, 2609-2626, 2609-2627, 2609-2628, 2610-2625, 2610-2626, 2610-2627, 2610-2628, 2610-2629, 2611-2626, 2611-2627, 2611-2629, 2611-2630, 2612-2627, 2612-2628, 2612-2629, 2612-2630, 2612-2631, 2613-2628, 2613-2629, 2613-2630, 2613-2631, 2614-2629, 2614-2630, 2614-2631, 2615-2630, 2615-2630, 2615-2631, and 2616-2631.

In certain embodiments, the following nucleotide regions of SEQ ID NO: 2, when targeted by antisense compounds or oligonucleotides, display at least 70% inhibition: 1685-1704, 1686-1705, 1769-1784, 1871-1890, 1873-1892, 1875-1890, 1875-1894, 1877-1892, 1877-1896, 1879-1894, 1879-1898, 3819-3838, 3825-3844, 3831-3850, 4151-4166, 5890-5909, 5904-5923, 5904-5923, 6406-6425, 6983-6998, 6983-7002, 6985-7000, 6985-7004, 7688-7707, 7690-7709, 7692-7707, 7692-7711, 7694-7709, 7696-7711, 7696-7715, 7786-7801, 7787-7806, 7788-7805, 7788-7806, 7788-7807, 7789-7806, 7789-7807, 7789-7808, 7790-7807, 7790-7809, 7791-7808, 7791-7809, 7791-7810, 7792-7809, 7792-7811, 7793-7810, 7793-7811, 7793-7812, 7794-7811, 7794-7812, 7794-7813, 7795-7812, 7795-7813, 7795-7814, 7796-7813, 7796-7814, 7796-7815, 7797-7812, 7797-7814, 7797-7816, 7798-7813, 7798-7815, 7798-7817, 7799-7816, 7799-7818, 7800-7819, 7801-7818, 7801-7820, 7802-7817, 7802-7819, 7802-7821, 7803-7820, 7803-7822, 7804-7821, 7804-7823, 7805-7822, 7805-7824, 7806-7823, 7806-7825, 7807-7824, 7807-7826, 7808-7825, 7808-7827, 7809-7826, 7809-7828, 7810-7827, 7811-7828, 7811-7830, 7812-7829, 7812-7831, 7813-7832, 7814-7833, 7815-7831, 7815-7833, 7815-7834, 7816-7832, 7816-7835, 7817-7833, 7817-7835, 7817-7836, 7818-7834, 7818-7836, 7818-7837, 7819-7835, 7819-7837, 7819-7838, 7820-7836, 7820-7838, 7820-7839, 7821-7840, 7822-7841, 7823-7839, 7823-7841, 7823-7842, 7824-7840, 7824-7841, 7824-7842, 7824-7843, 7825-7840, 7825-7841, 7825-7842, 7825-7844, 7826-7842, 7826-7845, 7827-7846, 7828-7843, 7828-7847, 7829-7844, 7829-7845, 7829-7847, 7829-7848, 7830-7845, 7830-7846, 7830-7847, 7830-7848, 7830-7849, 7831-7846, 7831-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7850, 7834-7851, 7834-7852, 7834-7853, 7835-7850, 7835-7851, 7835-7852, 7835-7853, 7835-7854, 7836-7851, 7836-7852, 7836-7853, 7836-7854, 7836-7855, 7837-7852, 7837-7853, 7837-7854, 7837-7855, 7837-7856, 7838-7854, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7861, 7843-7862, 7844-7859, 7844-7860, 7844-7861, 7844-7862, 7845-7860, 7845-7861, 7845-7862, 7846-7861, 7846-7862, and 7847-7862.

In certain embodiments, the following nucleotide regions of SEQ ID NO: 1, when targeted by antisense compounds or oligonucleotides, display at least 80% inhibition: 152-171, 154-169, 156-171, 158-173, 1135-1154, 1171-1186, 1173-1188, 1175-1190, 1763-1782, 1912-1931, 2197-2212, 2223-2238, 2225-2240, 2227-2242, 2457-2472, 2459-2474, 2461-2476, 2551-2566, 2553-2570, 2553-2571, 2553-2572, 2554-2573, 2555-2572, 2555-2574, 2556-2573, 2556-2574, 2556-2575, 2557-2574, 2557-2576, 2558-2575, 2558-2576, 2559-2577, 2559-2578, 2560-2577, 2560-2578, 2560-2579, 2561-2578, 2561-2579, 2561-2580, 2562-2577, 2562-2579, 2562-2581, 2563-2580, 2563-2582, 2564-2581, 2564-2583, 2565-2584, 2566-2583, 2567-2584, 2567-2586, 2568-2585, 2568-2587, 2569-2586, 2569-2588, 2570-2587, 2571-2588, 2571-2590, 2572-2589, 2572-2591, 2573-2590, 2573-2592, 2574-2591, 2574-2593, 2575-2592, 2576-2593, 2576-2595, 2577-2594, 2577-2596, 2578-2597, 2580-2598, 2580-2599, 2581-2597, 2581-2600, 2582-2601, 2583-2602, 2584-2603, 2585-2604, 2586-2605, 2587-2606, 2588-2607, 2589-2608, 2590-2606, 2590-2607, 2590-2609, 2591-2610, 2592-2611, 2593-2608, 2593-2612, 2594-2613, 2595-2611, 2595-2614, 2596-2615, 2597-2612, 2597-2613, 2597-2614, 2597-2615, 2597-2616, 2598-2613, 2598-2613, 2598-2614, 2598-2615, 2598-2616, 2598-2617, 2599-2614, 2599-2617, 2599-2618, 2600-2615, 2600-2617, 2600-2618, 2600-2619, 2601-2616, 2601-2617, 2601-2619, 2601-2620, 2602-2618, 2602-2621, 2603-2620, 2603-2621, 2603-2622, 2604-2619, 2604-2620, 2604-2621, 2604-2622, 2604-2623, 2605-2620, 2605-2621, 2605-2622, 2605-2623, 2605-2624, 2606-2621, 2606-2622, 2606-2623, 2606-2624, 2606-2625, 2607-2622, 2607-2623, 2607-2624, 2607-2625, 2607-2626, 2608-2623, 2608-2624, 2608-2625, 2608-2627, 2609-2624, 2609-2626, 2609-2627, 2609-2628, 2610-2625, 2610-2626, 2610-2628, 2610-2629, 2611-2626, 2611-2627, 2611-2629, 2611-2630, 2612-2627, 2612-2628, 2612-2630, 2612-2631, 2613-2628, 2613-2629, 2613-2631, 2614-2629, 2614-2630, 2614-2631, 2615-2630, and 2616-2631.

In certain embodiments, the following nucleotide regions of SEQ ID NO: 2, when targeted by antisense compounds or oligonucleotides, display at least 80% inhibition: 1685-1704, 1686-1705, 1873-1892, 1875-1890, 1877-1892, 1879-1894, 3819-3838, 4151-4166, 5904-5923, 6406-6425, 6985-7000, 7692-7707, 7694-7709, 7696-7711, 7786-7801, 7788-7805, 7788-7806, 7788-7807, 7789-7808, 7790-7807, 7790-7809, 7791-7808, 7791-7809, 7791-7810, 7792-7809, 7792-7811, 7793-7810, 7793-7811, 7794-7812, 7794-7813, 7795-7812, 7795-7813, 7795-7814, 7796-7813, 7796-7814, 7796-7815, 7797-7812, 7797-7814, 7797-7816, 7798-7815, 7798-7817, 7799-7816, 7799-7818, 7800-7819, 7801-7818, 7802-7819, 7802-7821, 7803-7820, 7803-7822, 7804-7821, 7804-7823, 7805-7822, 7806-7823, 7806-7825, 7807-7824, 7807-7826, 7808-7825, 7808-7827, 7809-7826, 7809-7828, 7810-7827, 7811-7828, 7812-7829, 7812-7831, 7813-7832, 7814-7833, 7815-7834, 7816-7832, 7816-7835, 7817-7836, 7818-7837, 7819-7838, 7820-7839, 7821-7840, 7822-7841, 7823-7842, 7824-7843, 7825-7841, 7825-7842, 7825-7844, 7826-7845, 7827-7846, 7828-7843, 7828-7847, 7829-7848, 7830-7846, 7830-7849, 7831-7850, 7832-7847, 7832-7848, 7832-7849, 7832-7850, 7832-7851, 7833-7848, 7833-7849, 7833-7850, 7833-7851, 7833-7852, 7834-7849, 7834-7852, 7834-7853, 7835-7850, 7835-7852, 7835-7853, 7835-7854, 7836-7851, 7836-7852, 7836-7854, 7836-7855, 7837-7853, 7837-7856, 7838-7855, 7838-7856, 7838-7857, 7839-7854, 7839-7855, 7839-7856, 7839-7857, 7839-7858, 7840-7855, 7840-7856, 7840-7857, 7840-7858, 7840-7859, 7841-7856, 7841-7857, 7841-7858, 7841-7859, 7841-7860, 7842-7857, 7842-7858, 7842-7859, 7842-7860, 7842-7861, 7843-7858, 7843-7859, 7843-7860, 7843-7862, 7844-7859, 7844-7861, 7844-7862, 7845-7860, 7845-7861, 7846-7862, and 7847-7862.

In certain embodiments, the following nucleotide regions of SEQ ID NO: 1, when targeted by antisense compounds or oligonucleotides, display at least 90% inhibition: 154-169, 156-171, 158-173, 1135-1154, 1171-1186, 1173-1188, 1763-1782, 1912-1931, 2223-2238, 2227-2242, 2459-2474, 2461-2476, 2554-2573, 2555-2574, 2560-2577, 2561-2578, 2561-2579, 2562-2581, 2563-2580, 2563-2582, 2564-2581, 2566-2583, 2567-2584, 2568-2585, 2568-2587, 2569-2586, 2570-2587, 2576-2593, 2577-2594, 2577-2596, 2578-2597, 2580-2599, 2581-2600, 2582-2601, 2583-2602, 2584-2603, 2586-2605, 2587-2605, 2587-2606, 2588-2607, 2589-2608, 2590-2607, 2590-2609, 2592-2611, 2595-2614, 2596-2615, 2597-2612, 2597-2613, 2597-2615, 2597-2616, 2598-2613, 2598-2613, 2598-2617, 2599-2614, 2599-2618, 2600-2615, 2600-2619, 2601-2617, 2601-2620, 2602-2621, 2603-2622, 2604-2623, 2605-2621, 2605-2622, 2605-2624, 2606-2625, 2607-2626, 2608-2623, 2608-2625, 2609-2628, 2611-2627, 2611-2630, 2612-2628, 2612-2631, 2613-2629, 2614-2629, 2615-2630, and 2616-2631.

In certain embodiments, the following nucleotide regions of SEQ ID NO: 2, when targeted by antisense compounds or oligonucleotides, display at least 90% inhibition: 1685-1704, 1686-1705, 1875-1890, 1877-1892, 1879-1894, 3819-3838, 5904-5923, 6406-6425, 7694-7709, 7696-7711, 7789-7808, 7790-7809, 7795-7812, 7795-7813, 7796-7813, 7796-7814, 7797-7814, 7797-7816, 7798-7815, 7798-7817, 7799-7816, 7801-7818, 7802-7819, 7803-7820, 7803-7822, 7804-7821, 7805-7822, 7811-7828, 7812-7829, 7812-7831, 7813-7832, 7815-7834, 7818-7837, 7819-7838, 7821-7840, 7822-7840, 7822-7841, 7825-7842, 7832-7847, 7832-7848, 7832-7850, 7833-7848, 7833-7852, 7834-7849, 7834-7853, 7835-7850, 7836-7852, 7836-7855, 7837-7856, 7838-7856, 7839-7857, 7839-7858, 7840-7856, 7840-7857, 7840-7859, 7843-7858, 7843-7860, and 7846-7862.

In certain embodiments, the following antisense compounds or oligonucleotides target a region of a CFB nucleic acid and effect at least a 50% inhibition of a CFB mRNA, ISIS NOs: 516350, 532614, 532632, 532635, 532638, 532639, 532686, 532687, 532688, 532689, 532690, 532691, 532692, 532692, 532693, 532694, 532695, 532696, 532697, 532698, 532699, 532700, 532701, 532702, 532703, 532704, 532705, 532706, 532707, 532770, 532775, 532778, 532780, 532791, 532800, 532809, 532810, 532811, 532917, 532952, 588509, 588510, 588511, 588512, 588513, 588514, 588515, 588516, 588517, 588518, 588519, 588520, 588522, 588523, 588524, 588525, 588527, 588528, 588529, 588530, 588531, 588532, 588533, 588534, 588535, 588536, 588537, 588538, 588539, 588540, 588541, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588566, 588567, 588568, 588569, 588570, 588571, 588572, 588573, 588574, 588575, 588576, 588577, 588580, 588581, 588585, 588586, 588589, 588590, 588599, 588603, 588606, 588608, 588610, 588614, 588616, 588628, 588631, 588632, 588634, 588636, 588638, 588640, 588645, 588646, 588654, 588656, 588658, 588660, 588662, 588664, 588670, 588672, 588676, 588682, 588688, 588696, 588698, 588807, 588808, 588809, 588813, 588814, 588815, 588819, 588820, 588822, 588823, 588838, 588839, 588840, 588841, 588842, 588846, 588847, 588848, 588849, 588850, 588851, 588852, 588853, 588854, 588855, 588856, 588857, 588858, 588859, 588860, 588861, 588862, 588863, 588864, 588865, 588866, 588867, 588868, 588870, 588871, 588872, 588873, 588874, 588875, 588876, 588877, 588878, 588879, 588880, 588881, 588882, 588883, 588884, 598999, 599000, 599001, 599002, 599003, 599004, 599005, 599006, 599007, 599008, 599009, 599010, 599011, 599012, 599013, 599014, 599015, 599018, 599019, 599023, 599024, 599025, 599026, 599027, 599028, 599029, 599030, 599031, 599032, 599033, 599034, 599035, 599058, 599062, 599063, 599064, 599065, 599070, 599071, 599072, 599073, 599074, 599076, 599077, 599078, 599079, 599080, 599081, 599082, 599083, 599084, 599085, 599086, 599087, 599088, 599089, 599090, 599091, 599092, 599093, 599094, 599095, 599096, 599097, 599098, 599102, 599119, 599123, 599124, 599125, 599126, 599127, 599128, 599132, 599133, 599134, 599135, 599136, 599137, 599138, 599139, 599140, 599141, 599142, 599143, 599144, 599145, 599147, 599148, 599149, 599150, 599151, 599152, 599153, 599154, 599155, 599156, 599157, 599158, 599159, 599178, 599179, 599180, 599181, 599182, 599186, 599187, 599188, 599189, 599190, 599191, 599192, 599193, 599194, 599195, 599196, 599197, 599198, 599199, 599200, 599201, 599202, 599203, 599204, 599205, 599206, 599207, 599208, 599209, 599210, 599211, 599212, 599213, 599214, 599215, 599216, 599217, 599218, 599219, 599220, 599221, 599221, 599222, 599223, 599224, 599225, 599226, 599227, 599228, 599229, 599230, 599231, 599232, 599233, 599234, 599235, 599236, 599241, 599247, 599248, 599249, 599255, 599256, 599257, 599258, 599260, 599261, 599262, 599263, 599264, 599265, 599266, 599267, 599268, 599269, 599270, 599271, 599272, 599273, 599274, 599275, 599276, 599277, 599278, 599279, 599280, 599297, 599299, 599306, 599307, 599308, 599309, 599311, 599312, 599313, 599314, 599315, 599316, 599317, 599318, 599319, 599320, 599321, 599322, 599323, 599324, 599325, 599326, 599327, 599328, 599329, 599330, 599338, 599349, 599353, 599354, 599355, 599356, 599357, 599358, 599359, 599360, 599361, 599362, 599363, 599364, 599369, 599371, 599372, 599373, 599376, 599378, 599379, 599382, 599383, 599384, 599385, 599386, 599387, 599388, 599389, 599390, 599391, 599392, 599393, 599394, 599395, 599396, 599397, 599398, 599399, 599400, 599401, 599402, 599403, 599404, 599405, 599406, 599407, 599408, 599409, 599410, 599412, 599413, 599414, 599415, 599416, 599417, 599418, 599419, 599420, 599421, 599422, 599423, 599424, 599425, 599426, 599433, 599434, 599435, 599436, 599437, 599438, 599439, 599440, 599441, 599442, 599443, 599444, 599445, 599446, 599447, 599448, 599450, 599454, 599455, 599456, 599467, 599468, 599469, 599471, 599472, 599473, 599474, 599475, 599476, 599477, 599478, 599479, 599480, 599481, 599482, 599483, 599484, 599485, 599486, 599487, 599488, 599489, 599490, 599491, 599492, 599493, 599494, 599495, 599496, 599497, 599498, 599499, 599500, 599501, 599502, 599503, 599504, 599505, 599506, 599507, 599508, 599509, 599512, 599515, 599518, 599531, 599541, 599541, 599546, 599547, 599548, 599549, 599550, 599552, 599553, 599554, 599555, 599557, 599558, 599561, 599562, 599563, 599564, 599565, 599566, 599567, 599568, 599569, 599570, 599577, 599578, 599579, 599580, 599581, 599581, 599582, 599584, 599585, 599586, 599587, 599588, 599589, 599590, 599591, 599592, 599593, 599594, 599595, 601321, 601322, 601323, 601325, 601327, 601328, 601329, 601330, 601332, 601333, 601334, 601335, 601336, 601337, 601338, 601339, 601341, 601342, 601343, 601344, 601345, 601346, 601347, 601348, 601349, 601362, 601367, 601368, 601369, 601371, 601372, 601373, 601374, 601375, 601377, 601378, 601380, 601381, 601382, 601383, 601384, 601385, 601386, 601387, and 601388.

In certain embodiments, the following antisense compounds or oligonucleotides target a region of a CFB nucleic acid and effect at least a 50% inhibition of a CFB mRNA, SEQ ID NOs: 12, 30, 33, 36, 37, 84, 85, 86, 87, 88, 89, 90, 90, 91, 92, 93, 94, 95, 96, 97, 98, 99, 100, 101, 102, 103, 104, 105, 198, 203, 206, 208, 219, 228, 237, 238, 239, 317, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 468, 472, 473, 475, 478, 479, 488, 492, 494, 495, 498, 499, 500, 502, 503, 509, 510, 511, 512, 513, 514, 515, 517, 518, 522, 523, 524, 525, 529, 530, 531, 534, 535, 537, 540, 541, 542, 543, 544, 545, 546, 547, 549, 550, 551, 552, 553, 554, 555, 556, 557, 558, 559, 563, 564, 565, 569, 570, 572, 573, 577, 588, 589, 590, 591, 592, 594, 595, 596, 597, 598, 599, 600, 601, 602, 603, 604, 605, 606, 607, 608, 609, 610, 611, 612, 613, 614, 615, 616, 617, 618, 619, 623, 640, 641, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 700, 704, 705, 706, 707, 708, 709, 711, 712, 713, 714, 715, 716, 717, 718, 720, 721, 722, 723, 724, 725, 726, 727, 728, 729, 730, 731, 732, 733, 734, 735, 736, 737, 738, 739, 740, 741, 742, 743, 744, 745, 745, 746, 747, 748, 749, 750, 751, 752, 753, 754, 755, 756, 758, 759, 760, 761, 762, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 813, 833, 834, 841, 846, 849, 850, 867, and 873.

In certain embodiments, the following antisense compounds or oligonucleotides target a region of a CFB nucleic acid and effect at least a 60% inhibition of a CFB mRNA, ISIS NOs: 516350, 532614, 532635, 532686, 532687, 532688, 532689, 532770, 532800, 532809, 532810, 532811, 532917, 532952, 588512, 588513, 588514, 588515, 588516, 588517, 588518, 588519, 588522, 588523, 588524, 588525, 588527, 588528, 588529, 588530, 588531, 588532, 588533, 588534, 588535, 588536, 588537, 588538, 588539, 588540, 588541, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588566, 588567, 588568, 588569, 588570, 588571, 588572, 588573, 588574, 588575, 588576, 588577, 588636, 588638, 588640, 588664, 588676, 588696, 588698, 588807, 588808, 588814, 588815, 588819, 588820, 588840, 588842, 588846, 588847, 588848, 588849, 588850, 588851, 588852, 588853, 588854, 588855, 588856, 588857, 588858, 588859, 588860, 588861, 588862, 588863, 588864, 588866, 588867, 588868, 588870, 588871, 588872, 588873, 588874, 588875, 588876, 588877, 588878, 588879, 588880, 588881, 588882, 588883, 588884, 598999, 599000, 599001, 599002, 599003, 599004, 599005, 599006, 599007, 599008, 599009, 599010, 599011, 599012, 599013, 599014, 599015, 599019, 599024, 599025, 599026, 599027, 599028, 599029, 599030, 599031, 599032, 599033, 599034, 599035, 599064, 599065, 599071, 599072, 599077, 599078, 599079, 599080, 599083, 599084, 599085, 599086, 599087, 599088, 599089, 599090, 599091, 599092, 599093, 599094, 599095, 599096, 599097, 599125, 599126, 599127, 599133, 599134, 599135, 599136, 599138, 599139, 599140, 599141, 599142, 599148, 599149, 599150, 599151, 599152, 599154, 599155, 599156, 599157, 599158, 599159, 599178, 599179, 599180, 599181, 599187, 599188, 599190, 599192, 599193, 599194, 599195, 599196, 599197, 599198, 599199, 599200, 599201, 599202, 599203, 599204, 599205, 599206, 599207, 599208, 599209, 599210, 599211, 599212, 599213, 599214, 599215, 599216, 599217, 599218, 599219, 599220, 599221, 599222, 599223, 599224, 599225, 599226, 599227, 599228, 599229, 599230, 599231, 599232, 599233, 599234, 599235, 599236, 599247, 599255, 599256, 599257, 599263, 599264, 599265, 599266, 599270, 599271, 599272, 599273, 599274, 599275, 599276, 599277, 599278, 599279, 599280, 599306, 599307, 599308, 599311, 599312, 599313, 599314, 599315, 599316, 599317, 599318, 599319, 599320, 599321, 599322, 599323, 599324, 599325, 599327, 599328, 599329, 599330, 599349, 599353, 599355, 599356, 599357, 599358, 599359, 599360, 599361, 599362, 599363, 599364, 599369, 599371, 599372, 599373, 599376, 599378, 599379, 599382, 599384, 599386, 599387, 599388, 599389, 599390, 599391, 599392, 599393, 599394, 599395, 599396, 599397, 599398, 599399, 599400, 599401, 599402, 599403, 599404, 599405, 599406, 599407, 599408, 599409, 599410, 599412, 599413, 599414, 599415, 599416, 599417, 599418, 599419, 599420, 599421, 599422, 599423, 599424, 599425, 599433, 599434, 599435, 599436, 599437, 599438, 599439, 599440, 599441, 599442, 599443, 599444, 599445, 599446, 599447, 599448, 599456, 599467, 599468, 599471, 599472, 599473, 599474, 599475, 599476, 599477, 599478, 599479, 599480, 599481, 599482, 599483, 599484, 599485, 599486, 599487, 599488, 599489, 599490, 599491, 599492, 599493, 599494, 599495, 599496, 599497, 599498, 599499, 599500, 599501, 599502, 599503, 599504, 599505, 599506, 599507, 599508, 599512, 599531, 599547, 599548, 599549, 599552, 599553, 599554, 599555, 599557, 599558, 599562, 599563, 599564, 599565, 599566, 599567, 599568, 599569, 599570, 599577, 599578, 599579, 599580, 599581, 599582, 599584, 599585, 599586, 599587, 599588, 599589, 599590, 599591, 599592, 599593, 599594, 599595, 601323, 601327, 601329, 601332, 601333, 601333, 601334, 601335, 601336, 601338, 601339, 601341, 601342, 601343, 601344, 601345, 601346, 601347, 601348, 601349, 601368, 601369, 601371, 601372, 601374, 601375, 601377, 601378, 601380, 601381, 601382, 601383, 601384, 601385, 601386, 601387, and 601388.

In certain embodiments, the following antisense compounds or oligonucleotides target a region of a CFB nucleic acid and effect at least a 60% inhibition of a CFB mRNA, SEQ ID NOs: 12, 33, 84, 85, 86, 87, 198, 228, 237, 238, 239, 317, 395, 396, 397, 398, 399, 400, 401, 402, 403, 404, 405, 406, 407, 408, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 472, 473, 513, 514, 515, 531, 537, 541, 542, 543, 544, 545, 546, 547, 549, 550, 551, 552, 553, 554, 555, 556, 557, 558, 564, 565, 569, 570, 577, 590, 592, 595, 596, 597, 598, 599, 600, 601, 602, 603, 604, 605, 606, 607, 608, 609, 610, 611, 612, 613, 614, 615, 616, 617, 618, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 682, 683, 684, 685, 686, 687, 688, 689, 700, 704, 706, 707, 708, 709, 711, 712, 713, 714, 715, 716, 717, 720, 721, 722, 723, 724, 725, 726, 727, 727, 728, 729, 730, 731, 732, 733, 734, 736, 737, 738, 739, 740, 741, 742, 743, 744, 745, 745, 746, 747, 748, 749, 750, 751, 752, 753, 754, 755, 756, 758, 759, 760, 761, 767, 768, 770, 772, 773, 774, 775, 775, 776, 776, 777, 777, 778, 779, 780, 781, 782, 783, 783, 784, 784, 785, 786, 787, 788, 789, 790, 791, 792, 793, 794, 795, 796, 797, 798, 799, 813, 833, 834, 841, 846, 849, and 850.

In certain embodiments, the following antisense compounds or oligonucleotides target a region of a CFB nucleic acid and effect at least a 70% inhibition of a CFB mRNA, ISIS NOs: 516350, 532614, 532686, 532687, 532688, 532770, 532800, 532809, 532810, 532811, 532917, 532952, 588512, 588513, 588514, 588515, 588516, 588517, 588518, 588524, 588529, 588530, 588531, 588532, 588533, 588534, 588535, 588536, 588537, 588538, 588539, 588540, 588541, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588568, 588569, 588570, 588571, 588572, 588573, 588574, 588575, 588577, 588636, 588638, 588640, 588696, 588698, 588807, 588814, 588815, 588819, 588842, 588847, 588848, 588849, 588850, 588851, 588852, 588853, 588856, 588857, 588858, 588859, 588860, 588861, 588862, 588863, 588866, 588867, 588870, 588871, 588872, 588873, 588874, 588875, 588876, 588877, 588878, 588879, 588880, 588881, 588882, 588883, 588884, 599000, 599001, 599003, 599004, 599005, 599008, 599009, 599010, 599011, 599014, 599015, 599024, 599025, 599027, 599028, 599029, 599030, 599031, 599032, 599033, 599034, 599072, 599077, 599080, 599085, 599086, 599087, 599088, 599089, 599090, 599091, 599093, 599094, 599095, 599096, 599097, 599125, 599126, 599134, 599138, 599139, 599148, 599149, 599150, 599151, 599152, 599154, 599155, 599156, 599157, 599158, 599187, 599188, 599193, 599195, 599196, 599197, 599198, 599199, 599200, 599201, 599202, 599203, 599204, 599205, 599206, 599207, 599208, 599210, 599211, 599212, 599213, 599214, 599215, 599216, 599217, 599218, 599219, 599220, 599221, 599222, 599223, 599224, 599225, 599226, 599227, 599228, 599229, 599230, 599231, 599232, 599233, 599234, 599235, 599236, 599266, 599272, 599272, 599273, 599274, 599275, 599277, 599278, 599279, 599280, 599280, 599306, 599311, 599312, 599313, 599314, 599315, 599316, 599317, 599318, 599319, 599320, 599321, 599322, 599323, 599325, 599327, 599328, 599329, 599330, 599355, 599357, 599358, 599359, 599360, 599361, 599362, 599363, 599364, 599369, 599371, 599372, 599373, 599378, 599379, 599382, 599384, 599386, 599387, 599388, 599389, 599390, 599391, 599392, 599393, 599394, 599395, 599396, 599397, 599398, 599399, 599400, 599401, 599402, 599403, 599404, 599405, 599406, 599407, 599408, 599409, 599410, 599413, 599414, 599415, 599416, 599417, 599418, 599419, 599420, 599421, 599422, 599423, 599424, 599433, 599434, 599435, 599436, 599437, 599438, 599439, 599440, 599441, 599442, 599443, 599445, 599446, 599447, 599448, 599472, 599473, 599474, 599475, 599476, 599477, 599478, 599479, 599480, 599481, 599482, 599483, 599484, 599485, 599486, 599487, 599488, 599489, 599490, 599491, 599492, 599493, 599494, 599495, 599496, 599497, 599498, 599499, 599500, 599501, 599502, 599503, 599504, 599505, 599506, 599507, 599508, 599512, 599547, 599548, 599552, 599553, 599554, 599555, 599558, 599562, 599563, 599564, 599566, 599567, 599568, 599569, 599570, 599577, 599578, 599579, 599580, 599581, 599582, 599585, 599586, 599587, 599588, 599589, 599590, 599591, 599592, 599593, 599594, 599595, 601332, 601335, 601341, 601343, 601344, 601345, 601346, 601347, 601348, 601349, 601371, 601372, 601380, 601382, 601383, 601384, 601385, 601386, and 601387.

In certain embodiments, the following antisense compounds or oligonucleotides target a region of a CFB nucleic acid and effect at least a 70% inhibition of a CFB mRNA, SEQ ID NOs: 12, 84, 85, 86, 198, 228, 237, 238, 239, 317, 395, 396, 397, 398, 399, 402, 403, 404, 405, 407, 408, 410, 411, 412, 412, 413, 414, 415, 416, 417, 418, 419, 420, 421, 422, 423, 424, 425, 426, 427, 428, 429, 430, 431, 432, 433, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 464, 465, 472, 473, 513, 514, 515, 541, 542, 543, 544, 545, 546, 547, 549, 550, 551, 552, 553, 554, 555, 556, 557, 564, 565, 569, 592, 595, 596, 597, 598, 599, 600, 601, 602, 603, 604, 606, 607, 608, 609, 610, 611, 612, 613, 614, 615, 616, 617, 618, 645, 646, 647, 648, 649, 650, 653, 654, 655, 656, 659, 660, 662, 663, 664, 665, 666, 668, 669, 670, 671, 672, 673, 674, 675, 676, 677, 677, 678, 679, 680, 682, 683, 684, 686, 687, 688, 689, 706, 708, 709, 711, 712, 713, 714, 715, 720, 721, 722, 723, 724, 725, 726, 727, 728, 729, 730, 731, 732, 733, 734, 736, 737, 738, 739, 740, 741, 742, 743, 744, 745, 746, 747, 748, 749, 750, 751, 752, 753, 754, 755, 756, 767, 768, 773, 775, 776, 777, 778, 779, 780, 781, 782, 783, 784, 785, 786, 787, 788, 789, 790, 791, 792, 793, 793, 794, 795, 797, 798, 799, 813, 833, 834, 841, 846, 849, 867, and 873.

In certain embodiments, the following antisense compounds or oligonucleotides target a region of a CFB nucleic acid and effect at least an 80% inhibition of a CFB mRNA, ISIS NOs: 532686, 532809, 532810, 532811, 532917, 532952, 588512, 588517, 588518, 588533, 588534, 588535, 588536, 588537, 588538, 588539, 588540, 588542, 588543, 588544, 588545, 588546, 588547, 588548, 588549, 588550, 588551, 588552, 588553, 588554, 588555, 588556, 588557, 588558, 588559, 588560, 588561, 588562, 588563, 588564, 588565, 588571, 588638, 588640, 588696, 588698, 588807, 588814, 588849, 588850, 588851, 588853, 588857, 588858, 588859, 588860, 588861, 588862, 588863, 588866, 588867, 588871, 588872, 588873, 588874, 588875, 588876, 588877, 588878, 588879, 588880, 588881, 588882, 588883, 599001, 599024, 599025, 599033, 599086, 599087, 599088, 599089, 599093, 599094, 599095, 599096, 599134, 599139, 599148, 599149, 599151, 599154, 599155, 599156, 599158, 599188, 599195, 599196, 599198, 599201, 599202, 599203, 599204, 599205, 599206, 599207, 599212, 599213, 599215, 599216, 599217, 599218, 599219, 599220, 599221, 599222, 599223, 599224, 599225, 599226, 599227, 599228, 599229, 599230, 599231, 599232, 599233, 599234, 599235, 599236, 599272, 599273, 599275, 599277, 599278, 599279, 599280, 599311, 599313, 599314, 599316, 599317, 599318, 599320, 599321, 599322, 599323, 599327, 599328, 599329, 599330, 599355, 599357, 599358, 599359, 599360, 599361, 599362, 599363, 599364, 599371, 599372, 599373, 599378, 599379, 599382, 599384, 599386, 599387, 599388, 599389, 599390, 599391, 599392, 599393, 599397, 599398, 599399, 599400, 599401, 599403, 599404, 599405, 599407, 599408, 599409, 599410, 599413, 599414, 599415, 599416, 599417, 599418, 599419, 599420, 599421, 599422, 599423, 599424, 599433, 599434, 599435, 599436, 599437, 599438, 599439, 599440, 599441, 599445, 599446, 599447, 599448, 599474, 599476, 599477, 599479, 599481, 599482, 599483, 599485, 599486, 599487, 599488, 599489, 599490, 599491, 599492, 599494, 599495, 599496, 599497, 599498, 599499, 599500, 599502, 599503, 599504, 599505, 599506, 599507, 599508, 599547, 599552, 599553, 599554, 599558, 599563, 599567, 599568, 599569, 599570, 599577, 599578, 599581, 599582, 599585, 599587, 599588, 599590, 599591, 599592, 599593, 599594, 601332, 601344, 601345, 601382, 601383, and 601385.

In certain embodiments, the following antisense compounds or oligonucleotides target a region of a CFB nucleic acid and effect at least a 80% inhibition of a CFB mRNA, SEQ ID NOs: 84, 237, 238, 239, 317, 395, 397, 411, 412, 413, 414, 415, 417, 418, 419, 420, 421, 422, 423, 425, 426, 427, 429, 430, 431, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 472, 473, 514, 515, 542, 543, 544, 545, 546, 547, 550, 551, 552, 553, 554, 555, 556, 557, 564, 595, 599, 600, 601, 602, 603, 606, 607, 608, 609, 610, 611, 612, 613, 614, 615, 616, 617, 618, 646, 655, 660, 662, 663, 666, 669, 670, 671, 672, 673, 675, 676, 677, 678, 679, 682, 684, 686, 687, 688, 689, 706, 708, 709, 711, 712, 713, 714, 715, 720, 722, 723, 724, 725, 726, 727, 729, 730, 731, 732, 733, 736, 737, 738, 739, 740, 741, 742, 743, 744, 745, 746, 747, 748, 749, 750, 751, 752, 753, 754, 755, 756, 768, 775, 776, 778, 781, 782, 783, 784, 785, 787, 788, 789, 790, 791, 792, 793, 794, 799, 813, 833, 834, 841, 849, 867, and 873.

In certain embodiments, the following antisense compounds or oligonucleotides target a region of a CFB nucleic acid and effect at least a 90% inhibition of a CFB mRNA, ISIS NOs: 532686, 532811, 532917, 588536, 588537, 588538, 588539, 588544, 588545, 588546, 588548, 588551, 588552, 588553, 588554, 588555, 588556, 588557, 588558, 588559, 588560, 588561, 588562, 588564, 588638, 588640, 588696, 588698, 588849, 588850, 588851, 588860, 588866, 588867, 588872, 588873, 588874, 588876, 588877, 588878, 588879, 588881, 588883, 599149, 599188, 599203, 599206, 599220, 599221, 599222, 599223, 599224, 599225, 599226, 599227, 599228, 599229, 599235, 599236, 599279, 599280, 599314, 599321, 599362, 599378, 599390, 599391, 599398, 599399, 599404, 599413, 599414, 599416, 599419, 599420, 599422, 599435, 599437, 599438, 599441, 599483, 599494, 599508, 599552, 599553, 599554, 599568, 599570, 599577, 599581, 599591, 599592, and 599593.

In certain embodiments, the following antisense compounds or oligonucleotides target a region of a CFB nucleic acid and effect at least a 90% inhibition of a CFB mRNA, SEQ ID NOs: 84, 238, 239, 317, 412, 413, 420, 421, 426, 434, 436, 437, 438, 439, 440, 442, 443, 444, 445, 446, 448, 451, 452, 453, 454, 455, 456, 457, 458, 459, 460, 461, 462, 464, 465, 472, 473, 514, 515, 542, 543, 544, 545, 546, 551, 553, 555, 556, 599, 600, 601, 602, 610, 616, 617, 618, 662, 666, 670, 676, 677, 678, 688, 689, 713, 723, 729, 730, 740, 741, 742, 743, 744, 745, 746, 747, 748, 749, 755, 756, 768, 783, 793, 833, and 867.

In certain embodiments, a compound can comprise or consist of any oligonucleotide targeted to CFB described herein and a conjugate group.

In certain embodiments, a compound comprises a modified oligonucleotide and a conjugate group, wherein the modified oligonucleotide consists of 10 to 30 linked nucleosides complementary within nucleotides 2193-2212, 2195-2210, 2457-2476, 2571-2590, 2584-2603, 2588-2607, 2592-2611, 2594-2613, 2597-2616, 2600-2619, or 2596-2611 of SEQ ID NO: 1.

In certain embodiments, a compound comprises a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598.

In certain embodiments, a compound comprises a modified oligonucleotide and a conjugate group, wherein the modified oligonucleotide has a nucleobase sequence consisting of any one of SEQ ID NOs: 198, 228, 237, 440, 444, 448, 450, 453, 455, 549, and 598.

In certain embodiments, any of the foregoing compounds or oligonucleotides can comprise at least one modified internucleoside linkage, at least one modified sugar, and/or at least one modified nucleobase.

In certain aspects, any of the foregoing compounds or oligonucleotides can comprise at least one modified sugar. In certain aspects, at least one modified sugar comprises a 2′-O-methoxyethyl group. In certain aspects, at least one modified sugar is a bicyclic sugar, such as a 4′-CH(CH₃)—O-2′ group, a 4′-CH₂—O-2′ group, or a 4′-(CH₂)₂—O-2′group.

In certain aspects, the modified oligonucleotide comprises at least one modified internucleoside linkage, such as a phosphorothioate internucleoside linkage.

In certain embodiments, the modified oligonucleotide comprises at least 1, 2, 3, 4, 5, 6, or 7 phosphodiester internucleoside linkages.

In certain embodiments, each internucleoside linkage of the modified oligonucleotide is selected from a phosphodiester internucleoside linkage and a phosphorothioate internucleoside linkage.

In certain embodiments, each internucleoside linkage of the modified oligonucleotide is a phosphorothioate linkage.

In certain embodiments, any of the foregoing compounds or oligonucleotides comprises at least one modified nucleobase, such as 5-methylcytosine.

In certain embodiments, a compound comprises a conjugate group and a modified oligonucleotide comprising:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar. In certain embodiments, the oligonucleotide consists of 10 to 30 linked nucleosides having a nucleobase sequence comprising the sequence recited in SEQ ID NO: 198, 228, 237, 440, 444, 448, 450, 453, 455, 549, or 598.

In certain embodiments, the modified oligonucleotide has a nucleobase sequence comprising or consisting of the sequence recited in SEQ ID NO: 198, 228, 237, 440, 444, 448, 450, 453, or 455, wherein the modified oligonucleotide comprises:
a gap segment consisting of ten linked deoxynucleosides;
a 5′ wing segment consisting of five linked nucleosides; and
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar; wherein each internucleoside linkage is a phosphorothioate linkage and wherein each cytosine is a 5-methylcytosine.

In certain embodiments, a compound comprises or consists of a single-stranded modified oligonucleotide and a conjugate group, wherein the modified oligonucleotide consists of 20 linked nucleosides having a nucleobase sequence consisting of the sequence recited in SEQ ID NO: 198, 228, 237, 440, 444, 448, 450, 453, or 455, wherein the oligonucleotide comprises:
a gap segment consisting of ten linked deoxynucleosides;
a 5′ wing segment consisting of five linked nucleosides; and
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar; wherein each internucleoside linkage is a phosphorothioate linkage; and wherein each cytosine is a 5-methylcytosine.

In certain embodiments, a compound comprises or consists of ISIS 588540 and a conjugate group. In certain embodiments, ISIS 588540 has the following chemical structure:

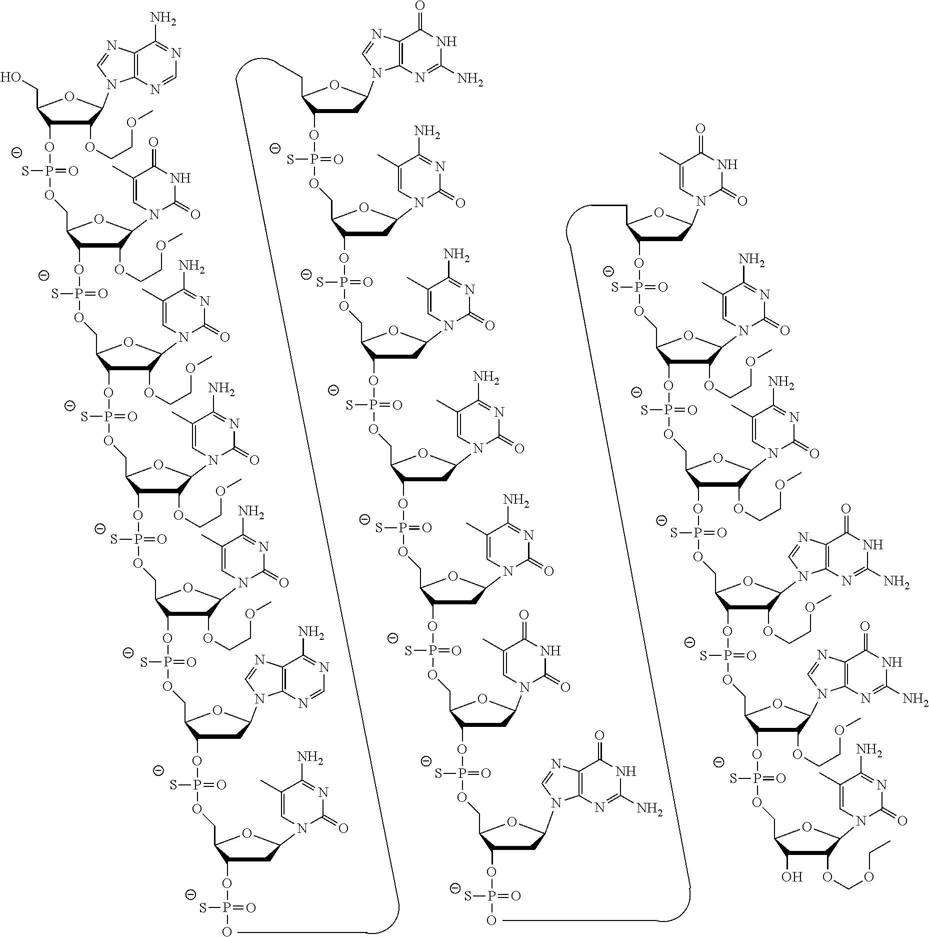

In certain embodiments, the modified oligonucleotide has a nucleobase sequence comprising or consisting of the sequence recited in SEQ ID NO: 549, wherein the modified oligonucleotide comprises
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment; wherein each nucleoside of each wing segment comprises a cEt sugar; wherein each internucleoside linkage is a phosphorothioate linkage; and wherein each cytosine is a 5-methylcytosine.

In certain aspects, the modified oligonucleotide has a nucleobase sequence comprising or consisting of the sequence recited in SEQ ID NO: 598, wherein the modified oligonucleotide comprises
a gap segment consisting of ten linked deoxynucleosides;
a 5′ wing segment consisting of three linked nucleosides; and
a 3′ wing segment consisting of three linked nucleosides;
wherein the gap segment is positioned between the 5′ wing segment and the 3′ wing segment; wherein the 5′ wing segment comprises a 2′-O-methoxyethyl sugar, 2′-O-methoxyethyl sugar, and cEt sugar in the 5′ to 3′ direction; wherein the 3′ wing segment comprises a cEt sugar, cEt sugar, and 2′-O-methoxyethyl sugar in the 5′ to 3′ direction; wherein each internucleoside linkage is a phosphorothioate linkage; and wherein each cytosine is a 5-methylcytosine.

In any of the foregoing embodiments, the compound or oligonucleotide can be at least 85%, at least 90%, at least 95%, at least 98%, at least 99%, or 100% complementary to a nucleic acid encoding CFB.

In any of the foregoing embodiments, the compound or oligonucleotide can be single-stranded.

In certain embodiments, the conjugate group is linked to the modified oligonucleotide at the 5′ end of the modified oligonucleotide. In certain embodiments, the conjugate group is linked to the modified oligonucleotide at the 3′ end of the modified oligonucleotide. In certain embodiments, the conjugate group comprises at least one N-Acetylgalactosamine (GalNAc), at least two N-Acetylgalactosamines (GalNAcs), or at least three N-Acetylgalactosamines (GalNAcs).

In certain embodiments, a compound having the following chemical structure comprises or consists of ISIS 588540 with a 5′-X, wherein X is a conjugate group comprising GalNAc as described herein:

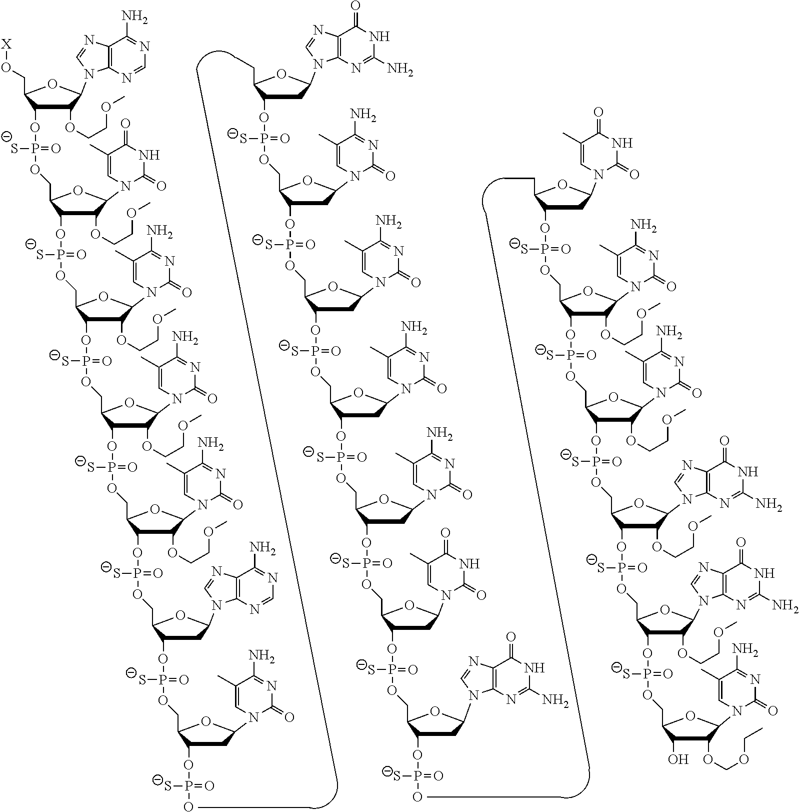

In certain embodiments, a compound comprises or consists of SEQ ID NO: 440, 5′-GalNAc, and chemical modifications as represented by the following chemical structure:

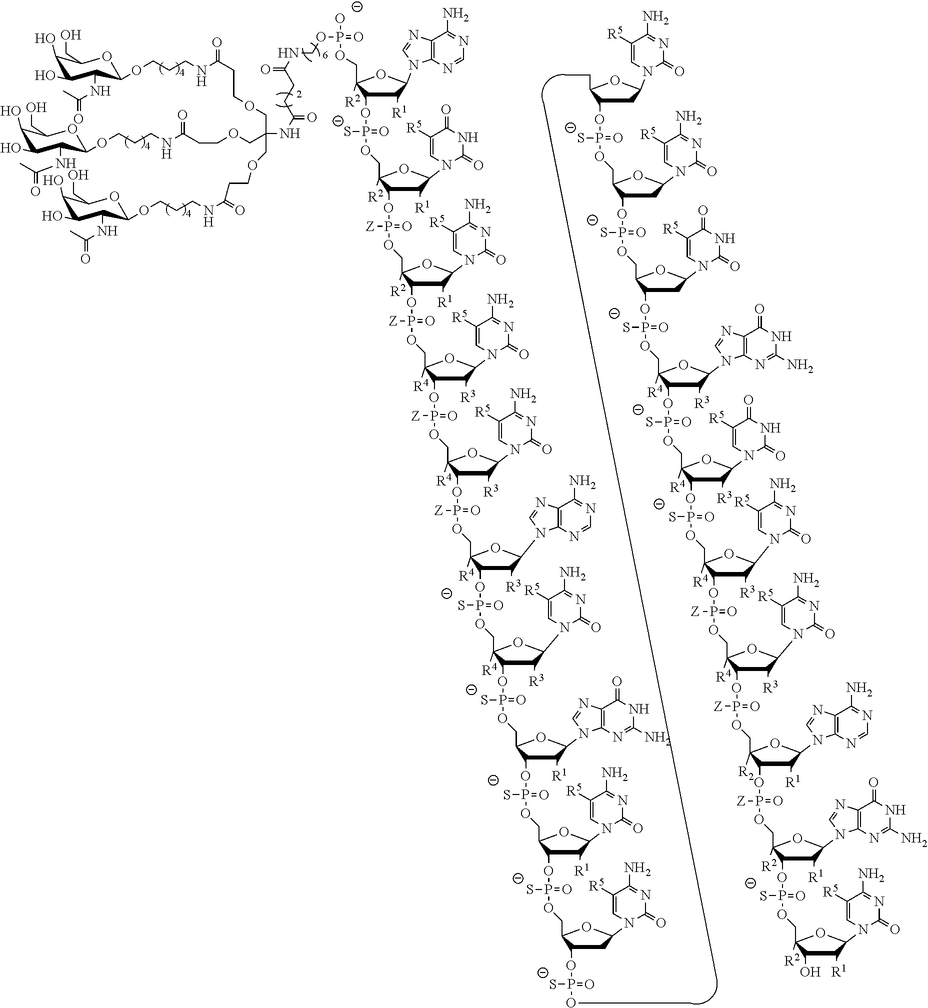

wherein either R¹ is —OCH₂CH₂OCH₃ (MOE) and R² is H; or R¹ and R² together form a bridge, wherein R¹ is —O— and R² is —CH₂—, —CH(CH₃)—, or —CH₂CH₂—, and R¹ and R² are directly connected such that the resulting bridge is selected from: —O—CH₂—, —O—CH(CH₃)—, and —O—CH₂CH₂—;
And for each pair of R³ and R₄ on the same ring, independently for each ring: either R₃ is selected from H and —OCH₂CH₂OCH₃ and R₄ is H; or R³ and R₄ together form a bridge, wherein R₃ is —O—, and R₄ is —CH₂—, —CH(CH₃)—, or —CH₂CH₂—and R₃ and R₄ are directly connected such that the resulting bridge is selected from: —O—CH₂—, —O—CH(CH₃)—, and —O—CH₂CH₂—;
And R⁵ is selected from H and —CH₃;
And Z is selected from S⁻ and O⁻.

In certain embodiments, a compound comprises ISIS 696844. In certain embodiments, a compound consists of ISIS 696844. In certain embodiments, ISIS 696844 has the following chemical structure:

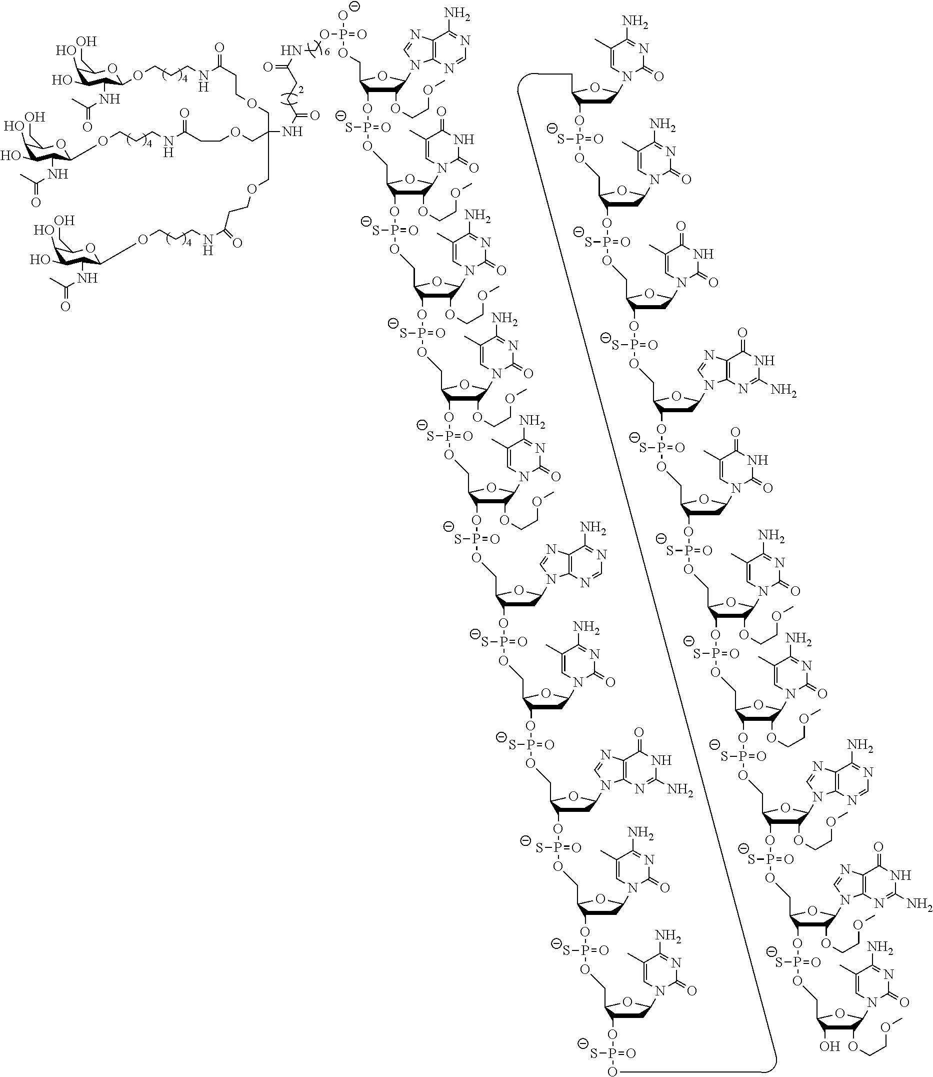

In certain embodiments, a compound comprises ISIS 696845. In certain embodiments, a compound consists of ISIS 696845. In certain embodiments, ISIS 696845 has the following chemical structure:

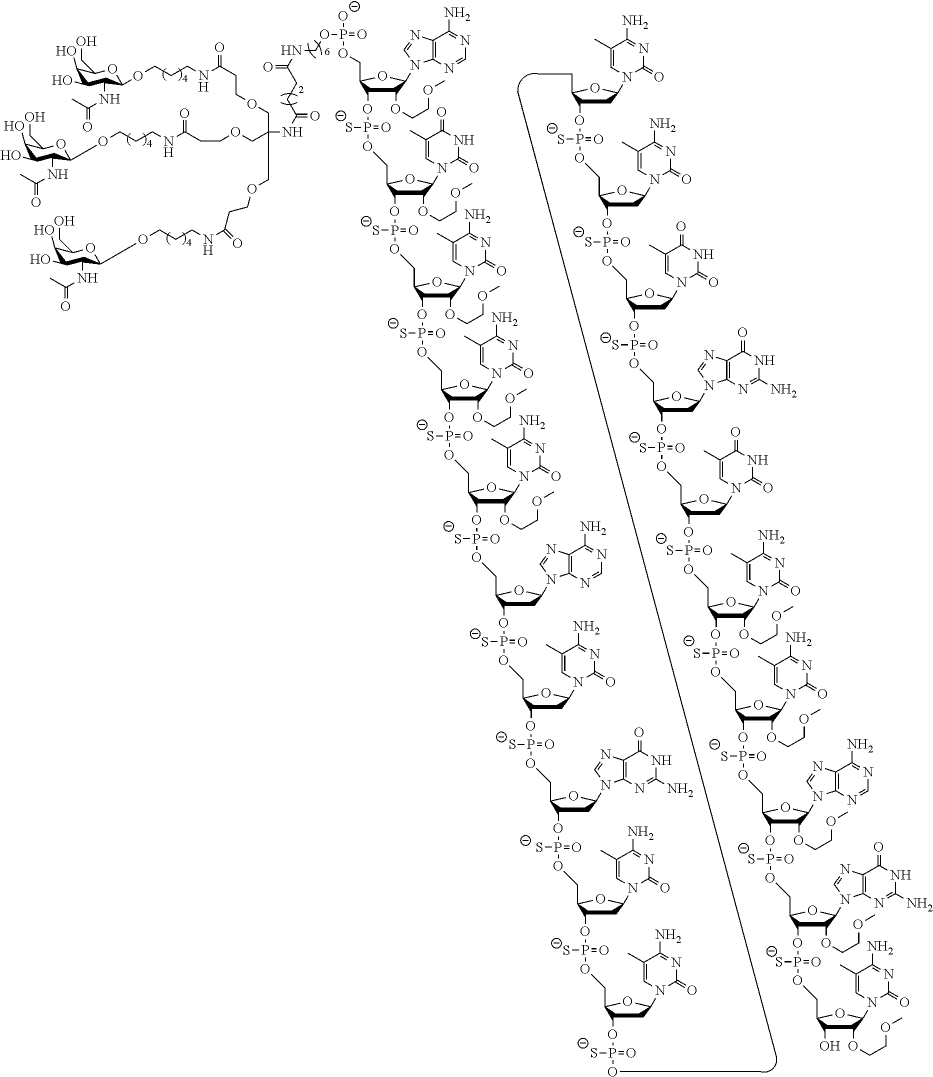

In certain embodiments, a compound comprises ISIS 698969. In certain embodiments, a compound consists of ISIS 698969. In certain embodiments, ISIS 698969 has the following chemical structure:

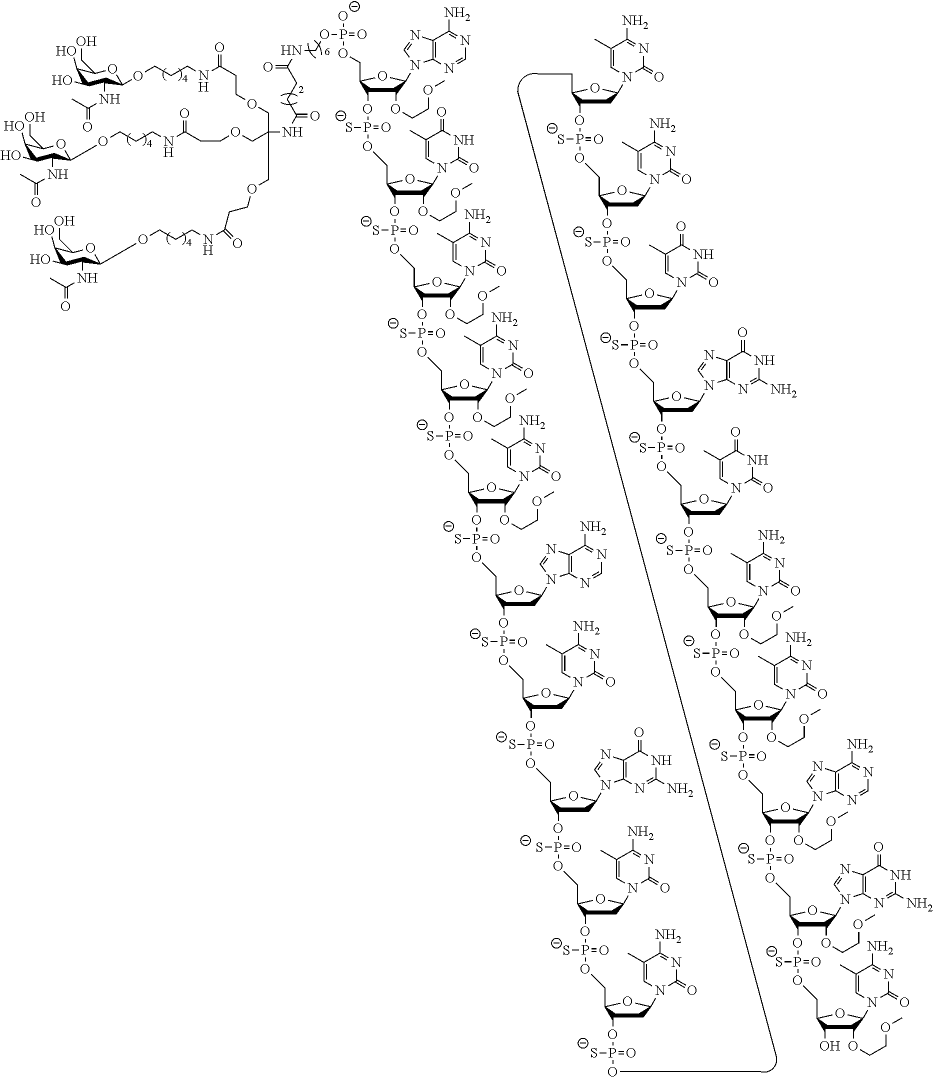

In certain embodiments, a compound comprises ISIS 698970. In certain embodiments, a compound consists of ISIS 698970. In certain embodiments, ISIS 698970 has the following chemical structure:

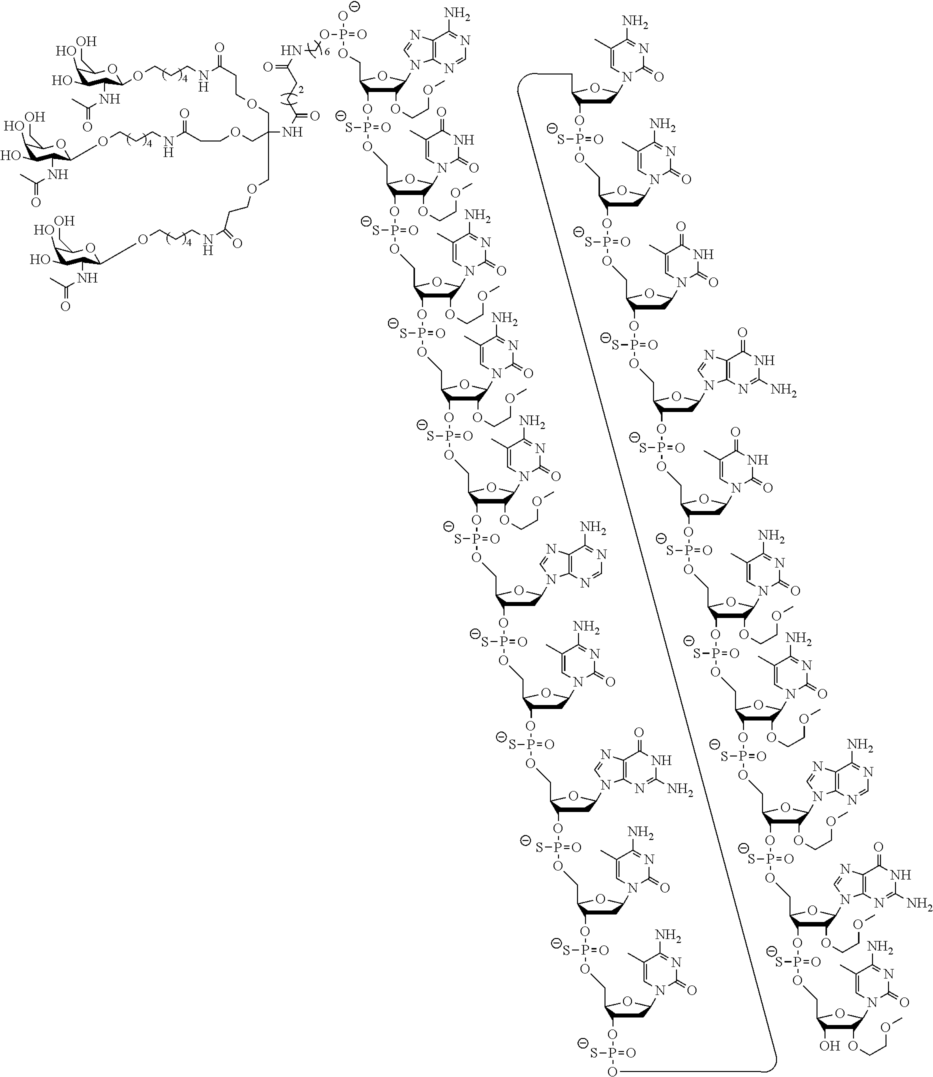

In certain embodiments, the compounds or compositions as described herein are efficacious by virtue of having at least one of an in vitro IC₅₀ of less than 250 nM, less than 200 nM, less than 150 nM, less than 100 nM, less than 90 nM, less than 80 nM, less than 70 nM, less than 65 nM, less than 60 nM, less than 55 nM, less than 50 nM, less than 45 nM, less than 40 nM, less than 35 nM, less than 30 nM, less than 25 nM, or less than 20 nM.

In certain embodiments, the compounds or compositions as described herein are highly tolerable as demonstrated by having at least one of an increase an ALT or AST value of no more than 4 fold, 3 fold, or 2 fold over saline treated animals or an increase in liver, spleen, or kidney weight of no more than 30%, 20%, 15%, 12%, 10%, 5%, or 2%. In certain embodiments, the compounds or compositions as described herein are highly tolerable as demonstrated by having no increase of ALT or AST over saline treated animals. In certain embodiments, the compounds or compositions as described herein are highly tolerable as demonstrated by having no increase in liver, spleen, or kidney weight over saline treated animals.

Certain embodiments provide a composition comprising the compound of any of the aforementioned embodiments or salt thereof and at least one of a pharmaceutically acceptable carrier or diluent. In certain aspects, the composition has a viscosity less than about 40 centipoise (cP), less than about 30 centipose (cP), less than about 20 centipose (cP), less than about 15 centipose (cP), or less than about 10 centipose (cP). In certain aspects, the composition having any of the aforementioned viscosities comprises a compound provided herein at a concentration of about 100 mg/mL, about 125 mg/mL, about 150 mg/mL, about 175 mg/mL, about 200 mg/mL, about 225 mg/mL, about 250 mg/mL, about 275 mg/mL, or about 300 mg/mL. In certain aspects, the composition having any of the aforementioned viscosities and/or compound concentrations has a temperature of room temperature or about 20° C., about 21° C., about 22° C., about 23° C., about 24° C., about 25° C., about 26° C., about 27° C., about 28° C., about 29° C., or about 30° C.

In certain embodiments, a method of treating, preventing, or ameliorating a disease associated with dysregulation of the complement alternative pathway in a subject comprises administering to the subject a compound or composition described herein, thereby treating, preventing, or ameliorating the disease. In certain aspects, the complement alternative pathway is activated greater than normal. In certain embodiments, a method of treating, preventing, or ameliorating a disease associated with dysregulation of the complement alternative pathway in a subject comprises administering to the subject a compound comprising or consisting of a modified oligonucleotide and a conjugate group, wherein the modified oligonucleotide consists of 10 to 30 linked nucleosides and has a nucleobase sequence comprising the nucleobase sequence of any one of SEQ ID NOs: 6-808. In certain embodiments, a method of treating, preventing, or ameliorating a disease associated with dysregulation of the complement alternative pathway in a subject comprises administering to the subject a compound comprising or consisting of a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598. In certain embodiments, a method of treating, preventing, or ameliorating a disease associated with dysregulation of the complement alternative pathway in a subject comprises administering to the subject a compound comprising or consisting of ISIS 696844, ISIS 696845, ISIS 698969, or ISIS 698970.

In certain embodiments, a method of treating, preventing, or ameliorating macular degeneration, such as age-related macular degeneration (AMD) in a subject comprises administering to the subject a compound or composition described herein, thereby treating, preventing, or ameliorating AMD. In certain aspects, the complement alternative pathway is activated greater than normal. In certain aspects, the AMD is wet AMD.

In certain aspects, the AMD is dry AMD, such as Geographic Atrophy. In certain embodiments, a method of treating, preventing, or ameliorating macular degeneration in a subject, such as age-related macular degeneration (AMD), wet AMD, dry AMD, or Geographic Atrophy comprises administering to the subject a a compound comprising or consisting of a modified oligonucleotide and a conjugate group, wherein the modified oligonucleotide consists of 10 to 30 linked nucleosides and has a nucleobase sequence comprising the nucleobase sequence of any one of SEQ ID NOs: 6-808. In certain embodiments, a method of treating, preventing, or ameliorating macular degeneration, such as age-related macular degeneration (AMD), wet AMD, dry AMD, or Geographic Atrophy in a subject comprises administering to the subject a comprises administering to the subject a compound comprising or consisting of a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598. In certain embodiments, a method of treating, preventing, or ameliorating macular degeneration, such as age-related macular degeneration (AMD), wet AMD, dry AMD, or Geographic Atrophy in a subject comprises administering to the subject a compound comprising or consisting of ISIS 696844, ISIS 696845, ISIS 698969, or ISIS 698970. In certain aspects, the compound or composition is administered to the subject parenterally.

In certain embodiments, a method of treating, preventing, or ameliorating a kidney disease associated with dysregulation of the complement alternative pathway in a subject comprises administering to the subject a compound or composition described herein, thereby treating, preventing, or ameliorating the kidney disease. In certain embodiments, a method of treating, preventing, or ameliorating a kidney disease associated with dysregulation of the complement alternative pathway in a subject comprises administering to the subject a compound comprising or consisting of a modified oligonucleotide and a conjugate group, wherein the modified oligonucleotide consists of 10 to 30 linked nucleosides and has a nucleobase sequence comprising the nucleobase sequence of any one of SEQ ID NOs: 6-808. In certain embodiments, a method of treating, preventing, or ameliorating a kidney disease associated with dysregulation of the complement alternative pathway in a subject comprises administering to the subject a compound comprising or consisting of a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598. In certain embodiments, a method of treating, preventing, or ameliorating a kidney disease associated with dysregulation of the complement alternative pathway in a subject comprises administering to the subject a compound comprising or consisting of ISIS 696844, ISIS 696845, ISIS 698969, or ISIS 698970. In certain aspects, the complement alternative pathway is activated greater than normal. In certain aspects, the kidney disease is lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof. In certain aspects, the kidney disease is associated with C3 deposits, such as C3 deposits in the glomerulus. In certain aspects, the kidney disease is associated with lower than normal circulating C3 levels, such as serum or plasma C3 levels. In certain aspects, administering the compound or composition reduces or inhibits accumulation of ocular C3 levels, such as C3 protein levels. In certain aspects, administering the compound or composition reduces the level of ocular C3 deposits or inhibits accumulation of ocular C3 deposits. In certain aspects, the compound or composition is administered to the subject parenterally. In certain aspects, administering the compound or composition reduces or inhibits accumulation of C3 levels in the kidney, such as C3 protein levels. In certain aspects, administering the compound or composition reduces the level of kidney C3 deposits or inhibits accumulation of kidney C3 deposits, such as C3 levels in the glomerulus. In certain aspects, the subject is identified as having or at risk of having a disease associated with dysregulation of the complement alternative pathway, for example by detecting complement levels or membrane-attack complex levels in the subject's blood and/or performing a genetic test for gene mutations of complement factors associated with the disease.

In certain embodiments, a method of inhibiting expression of Complement Factor B (CFB) in a subject having, or at risk of having, a disease associated with dysregulation of the complement alternative pathway comprises administering a compound or composition described herein to the subject, thereby inhibiting expression of CFB in the subject. In certain embodiments, a method of inhibiting expression of Complement Factor B (CFB) in a subject having, or at risk of having, a disease associated with dysregulation of the complement alternative pathway comprises administering to the subject a compound comprising or consisting of a modified oligonucleotide and a conjugate group, wherein the modified oligonucleotide consists of 10 to 30 linked nucleosides and has a nucleobase sequence comprising the nucleobase sequence of any one of SEQ ID NOs: 6-808. In certain embodiments, a method of inhibiting expression of Complement Factor B (CFB) in a subject having, or at risk of having, a disease associated with dysregulation of the complement alternative pathway comprises administering to the subject a compound comprising or consisting of a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598. In certain embodiments, a method of inhibiting expression of Complement Factor B (CFB) in a subject having, or at risk of having, a disease associated with dysregulation of the complement alternative pathway comprises administering to the subject a compound comprising or consisting of ISIS 696844, ISIS 696845, ISIS 698969, or ISIS 698970. In certain aspects, administering the compound or composition inhibits expression of CFB in the eye. In certain aspects, the subject has, or is at risk of having, age related macular degeneration (AMD), such as wet AMD and dry AMD. In certain aspects, dry AMD can be Geographic Atrophy. Geographic Atrophy is considered an advanced form of dry AMD involving degeneration of the retina. In certain aspects, administering the compound or composition inhibits expression of CFB in the kidney, such as in the glomerulus. In certain aspects, the subject has, or is at risk of having, lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof.

In certain embodiments, a method of reducing or inhibiting accumulation of C3 deposits in the eye of a subject having, or at risk of having, a disease associated with dysregulation of the complement alternative pathway comprises administering a compound or composition described herein to the subject, thereby reducing or inhibiting accumulation of C3 deposits in the eye of the subject. In certain embodiments, a method of reducing or inhibiting accumulation of C3 deposits in the eye of a subject having, or at risk of having, a disease associated with dysregulation of the complement alternative pathway comprises administering to the subject a compound comprising or consisting of a modified oligonucleotide and a conjugate group, wherein the modified oligonucleotide consists of 10 to 30 linked nucleosides and has a nucleobase sequence comprising the nucleobase sequence of any one of SEQ ID NOs: 6-808. In certain embodiments, a method of reducing or inhibiting accumulation of C3 deposits in the eye of a subject having, or at risk of having, a disease associated with dysregulation of the complement alternative pathway comprises administering to the subject a compound comprising or consisting of a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598. In certain embodiments, a method of reducing or inhibiting accumulation of C3 deposits in the eye of a subject having, or at risk of having, a disease associated with dysregulation of the complement alternative pathway comprises administering to the subject a compound comprising or consisting of ISIS 696844, ISIS 696845, ISIS 698969, or ISIS 698970. In certain aspects, the subject has, or is at risk of having, age related macular degeneration (AMD), such as wet AMD and dry AMD. In certain aspects, dry AMD can be Geographic Atrophy. In certain aspects, the compound or composition is administered to the subject parenterally.

In certain embodiments, a method of reducing or inhibiting accumulation of C3 deposits in the kidney of a subject having, or at risk of having, a disease associated with dysregulation of the complement alternative pathway comprises administering a compound or composition described herein to the subject, thereby reducing or inhibiting accumulation of C3 deposits in the kidney of the subject. In certain embodiments, a method of reducing or inhibiting accumulation of C3 deposits in the kidney of a subject having, or at risk of having, a disease associated with dysregulation of the complement alternative pathway comprises administering to the subject a compound comprising or consisting of a modified oligonucleotide and a conjugate group, wherein the modified oligonucleotide consists of 10 to 30 linked nucleosides and has a nucleobase sequence comprising the nucleobase sequence of any one of SEQ ID NOs: 6-808. In certain embodiments, a method of reducing or inhibiting accumulation of C3 deposits in the kidney of a subject having, or at risk of having, a disease associated with dysregulation of the complement alternative pathway comprises administering to the subject a compound comprising or consisting of a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598. In certain embodiments, a method of reducing or inhibiting accumulation of C3 deposits in the kidney of a subject having, or at risk of having, a disease associated with dysregulation of the complement alternative pathway comprises administering to the subject a compound comprising or consisting of ISIS 696844, ISIS 696845, ISIS 698969, or ISIS 698970. In certain aspects, the subject has, or is at risk of having, lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof. In certain aspects, the compound or composition is administered to the subject parenterally.

Certain embodiments are drawn to use of a compound or composition described herein for treating a disease associated with dysregulation of the complement alternative pathway. Certain embodiments are drawn to use of a compound comprising or consisting of a modified oligonucleotide and a conjugate group, wherein the modified oligonucleotide consists of 10 to 30 linked nucleosides and has a nucleobase sequence comprising the nucleobase sequence of any one of SEQ ID NOs: 6-808, for treating a disease associated with dysregulation of the complement alternative pathway. Certain embodiments are drawn to use of a compound comprising or consisting of a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598, for treating a disease associated with dysregulation of the complement alternative pathway. Certain embodiments are drawn to use of a compound comprising or consisting of ISIS 696844, ISIS 696845, ISIS 698969, or ISIS 698970 for treating a disease associated with dysregulation of the complement alternative pathway. In certain aspects, the complement alternative pathway is activated greater than normal. In certain aspects, the disease is macular degeneration, such as age related macular degeneration (AMD), which can be wet AMD or dry AMD. In certain aspects, dry AMD can be Geographic Atrophy. In certain aspects, the disease is a kidney disease such as lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof. In certain aspects, the compound or composition is administered to the subject parenterally.

In certain embodiments, a compound or composition described herein is administered parenterally. For example, in certain embodiments the compound or composition can be administered through injection or infusion. Parenteral administration includes subcutaneous administration, intravenous administration, intramuscular administration, intraarterial administration, intraperitoneal administration, or intracranial administration, e.g. intrathecal or intracerebroventricular administration.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted.

In certain embodiments, an antisense compound is 10 to 30 subunits in length. In certain embodiments, an antisense compound is 12 to 30 subunits in length. In certain embodiments, an antisense compound is 12 to 22 subunits in length. In certain embodiments, an antisense compound is 14 to 30 subunits in length. In certain embodiments, an antisense compound is 14 to 20 subunits in length. In certain embodiments, an antisense compound is 15 to 30 subunits in length. In certain embodiments, an antisense compound is 15 to 20 subunits in length. In certain embodiments, an antisense compound is 16 to 30 subunits in length. In certain embodiments, an antisense compound is 16 to 20 subunits in length. In certain embodiments, an antisense compound is 17 to 30 subunits in length. In certain embodiments, an antisense compound is 17 to 20 subunits in length. In certain embodiments, an antisense compound is 18 to 30 subunits in length. In certain embodiments, an antisense compound is 18 to 21 subunits in length. In certain embodiments, an antisense compound is 18 to 20 subunits in length. In certain embodiments, an antisense compound is 20 to 30 subunits in length. In other words, such antisense compounds are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, an antisense compound is 14 subunits in length. In certain embodiments, an antisense compound is 16 subunits in length. In certain embodiments, an antisense compound is 17 subunits in length. In certain embodiments, an antisense compound is 18 subunits in length. In certain embodiments, an antisense compound is 19 subunits in length. In certain embodiments, an antisense compound is 20 subunits in length. In other embodiments, the antisense compound is 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleotides.

In certain embodiments antisense oligonucleotides may be shortened or truncated. For example, a single subunit may be deleted from the 5′ end (5′ truncation), or alternatively from the 3′ end (3′ truncation). A shortened or truncated antisense compound targeted to an CFB nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al. (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358,1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Certain Antisense Compound Motifs and Mechanisms

In certain embodiments, antisense compounds have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may confer another desired property e.g., serve as a substrate for the cellular endonuclease RNase H, which cleaves the RNA strand of an RNA:DNA duplex.

Antisense activity may result from any mechanism involving the hybridization of the antisense compound (e.g., oligonucleotide) with a target nucleic acid, wherein the hybridization ultimately results in a biological effect. In certain embodiments, the amount and/or activity of the target nucleic acid is modulated. In certain embodiments, the amount and/or activity of the target nucleic acid is reduced. In certain embodiments, hybridization of the antisense compound to the target nucleic acid ultimately results in target nucleic acid degradation. In certain embodiments, hybridization of the antisense compound to the target nucleic acid does not result in target nucleic acid degradation. In certain such embodiments, the presence of the antisense compound hybridized with the target nucleic acid (occupancy) results in a modulation of antisense activity. In certain embodiments, antisense compounds having a particular chemical motif or pattern of chemical modifications are particularly suited to exploit one or more mechanisms. In certain embodiments, antisense compounds function through more than one mechanism and/or through mechanisms that have not been elucidated. Accordingly, the antisense compounds described herein are not limited by particular mechanism.

Antisense mechanisms include, without limitation, RNase H mediated antisense; RNAi mechanisms, which utilize the RISC pathway and include, without limitation, siRNA, ssRNA and microRNA mechanisms; and occupancy based mechanisms. Certain antisense compounds may act through more than one such mechanism and/or through additional mechanisms.

### RNase H-Mediated Antisense

In certain embodiments, antisense activity results at least in part from degradation of target RNA by RNase H. RNase H is a cellular endonuclease that cleaves the RNA strand of an RNA:DNA duplex. It is known in the art that single-stranded antisense compounds which are “DNA-like” elicit RNase H activity in mammalian cells. Accordingly, antisense compounds comprising at least a portion of DNA or DNA-like nucleosides may activate RNase H, resulting in cleavage of the target nucleic acid. In certain embodiments, antisense compounds that utilize RNase H comprise one or more modified nucleosides. In certain embodiments, such antisense compounds comprise at least one block of 1-8 modified nucleosides. In certain such embodiments, the modified nucleosides do not support RNase H activity. In certain embodiments, such antisense compounds are gapmers, as described herein. In certain such embodiments, the gap of the gapmer comprises DNA nucleosides. In certain such embodiments, the gap of the gapmer comprises DNA-like nucleosides. In certain such embodiments, the gap of the gapmer comprises DNA nucleosides and DNA-like nucleosides.

Certain antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2-modified nucleosides (such 2′-modified nucleosides may include 2′-MOE and 2′-O—CH₃, among others), and bicyclic sugar modified nucleosides (such bicyclic sugar modified nucleosides may include those having a constrained ethyl). In certain embodiments, nucleosides in the wings may include several modified sugar moieties, including, for example 2′-MOE and bicyclic sugar moieties such as constrained ethyl or LNA. In certain embodiments, wings may include several modified and unmodified sugar moieties. In certain embodiments, wings may include various combinations of 2′-MOE nucleosides, bicyclic sugar moieties such as constrained ethyl nucleosides or LNA nucleosides, and 2′-deoxynucleosides.

Each distinct region may comprise uniform sugar moieties, variant, or alternating sugar moieties. The wing-gap-wing motif is frequently described as “X—Y—Z”, where “X” represents the length of the 5′-wing, “Y” represents the length of the gap, and “Z” represents the length of the 3′-wing. “X” and “Z” may comprise uniform, variant, or alternating sugar moieties. In certain embodiments, “X” and “Y” may include one or more 2′-deoxynucleosides. “Y” may comprise 2′-deoxynucleosides. As used herein, a gapmer described as “X—Y—Z” has a configuration such that the gap is positioned immediately adjacent to each of the 5′-wing and the 3′ wing. Thus, no intervening nucleotides exist between the 5′-wing and gap, or the gap and the 3′-wing. Any of the antisense compounds described herein can have a gapmer motif. In certain embodiments, “X” and “Z” are the same; in other embodiments they are different. In certain embodiments, “Y” is between 8 and 15 nucleosides. X, Y, or Z can be any of 1, 2, 3, 4, 5, 6, 7, 8, 9, 10, 11, 12, 13, 14, 15, 16, 17, 18, 19, 20, 25, 30 or more nucleosides.

In certain embodiments, the antisense compound targeted to a CFB nucleic acid has a gapmer motif in which the gap consists of 6, 7, 8, 9, 10, 11, 12, 13, 14, 15, or 16 linked nucleosides.

In certain embodiments, the antisense oligonucleotide has a sugar motif described by Formula A as follows: (J)ₘ-(B)ₙ-(J)ₚ-(B)ᵣ-(A)ₜ-(D)_{g}-(A)ᵥ-(B)_{w}-(J)ₓ-(B)_{y}-(J)_{z}
wherein:
each A is independently a 2′-substituted nucleoside;
each B is independently a bicyclic nucleoside;
each J is independently either a 2′-substituted nucleoside or a 2′-deoxynucleoside;
each D is a 2′-deoxynucleoside;
m is 0-4; n is 0-2; p is 0-2; r is 0-2; t is 0-2; v is 0-2; w is 0-4; x is 0-2; y is 0-2; z is 0-4; g is 6-14; provided that:
at least one of m, n, and r is other than 0;
at least one of w and y is other than 0;
the sum of m, n, p, r, and t is from 2 to 5; and
the sum of v, w, x, y, and z is from 2 to 5.

### RNAi Compounds

In certain embodiments, antisense compounds are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). In certain embodiments, antisense compounds comprise modifications that make them particularly suited for such mechanisms.

i. SsRNA Compounds

In certain embodiments, antisense compounds including those particularly suited for use as single-stranded RNAi compounds (ssRNA) comprise a modified 5′-terminal end. In certain such embodiments, the 5′-terminal end comprises a modified phosphate moiety. In certain embodiments, such modified phosphate is stabilized (e.g., resistant to degradation/cleavage compared to unmodified 5′-phosphate). In certain embodiments, such 5′-terminal nucleosides stabilize the 5′-phosphorous moiety. Certain modified 5′-terminal nucleosides may be found in the art, for example in WO/2011/139702.

In certain embodiments, the 5′-nucleoside of an ssRNA compound has Formula IIc:

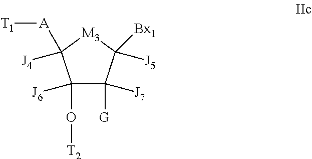

wherein:
   T₁ is an optionally protected phosphorus moiety;
   T₂ is an internucleoside linking group linking the compound of Formula IIc to the oligomeric compound;
   A has one of the formulas:

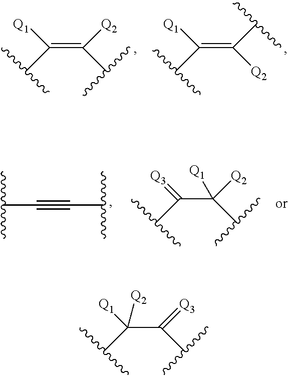

Q₁ and Q₂ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl, substituted C₂-C₆ alkynyl or N(R₃)(R₄);
Q₃ is O, S, N(R₅) or C(R₆)(R₇);
each R₃, R₄ R₅, R₆ and R₇ is, independently, H, C₁-C₆ alkyl, substituted C₁-C₆ alkyl or C₁-C₆ alkoxy;
M₃ is O, S, NR₁₄, C(R₁₅)(R₁₆), C(R₁₅)(R₁₆)C(R₁₇)(R₁₈), C(R₁₅)═C(R₁₇), OC(R₁₅)(R₁₆) or OC(R₁₅)(Bx₂);
R₁₄ is H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
R₁₅, R₁₆, R₁₇ and R₁ₛ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
Bx₁ is a heterocyclic base moiety;
or if Bx₂ is present then Bx₂ is a heterocyclic base moiety and Bx₁ is H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
J₄, J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
or J₄ forms a bridge with one of J₅ or J₇ wherein said bridge comprises from 1 to 3 linked biradical groups selected from O, S, NR₁₉, C(R₂₀)(R₂₁), C(R₂₀)═C(R₂₁), C[═C(R₂₀)(R₂₁)] and C(═O) and the other two of J₅, J₆ and J₇ are each, independently, H, halogen, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
each R₁₉, R₂₀ and R₂₁ is, independently, H, C₁-C₆ alkyl, substituted C₁-C₆ alkyl, C₁-C₆ alkoxy, substituted C₁-C₆ alkoxy, C₂-C₆ alkenyl, substituted C₂-C₆ alkenyl, C₂-C₆ alkynyl or substituted C₂-C₆ alkynyl;
G is H, OH, halogen or O—[C(R₈)(R₉)]ₙ—[(C═O)ₘ—X₁]ⱼ—Z;
each R₈ and R₉ is, independently, H, halogen, C₁-C₆ alkyl or substituted C₁-C₆ alkyl;
X₁ is O, S or N(E₁);
Z is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to about 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(═X₂)J₁, OC(═X₂)N(J₁)(J₂) and C(═X₂)N(J₁)(J₂);
X₂ is O, S or NJ₃;
each J₁, J₂ and J₃ is, independently, H or C₁-C₆ alkyl;
when j is 1 then Z is other than halogen or N(E₂)(E₃); and
wherein said oligomeric compound comprises from 8 to 40 monomeric subunits and is hybridizable to at least a portion of a target nucleic acid.

In certain embodiments, M₃ is O, CH═CH, OCH₂ or OC(H)(Bx₂). In certain embodiments, M₃ is O.

In certain embodiments, J₄, J₅, J₆ and J₇ are each H. In certain embodiments, J₄ forms a bridge with one of J₅ or J₇.

In certain embodiments, A has one of the formulas:

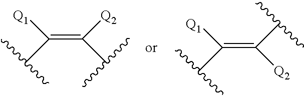

wherein:
   Q₁ and Q₂ are each, independently, H, halogen, C₁-C₆ alkyl, substituted C₁-C₆ alkyl, C₁-C₆ alkoxy or substituted C₁-C₆ alkoxy. In certain embodiments, Q₁ and Q₂ are each H. In certain embodiments, Q₁ and Q₂ are each, independently, H or halogen. In certain embodiments, Q₁ and Q₂ is H and the other of Q₁ and Q₂ is F, CH₃ or OCH₃.

In certain embodiments, T₁ has the formula:

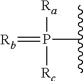

wherein:
   Rₐ and R_{c} are each, independently, protected hydroxyl, protected thiol, C₁-C₆ alkyl, substituted C₁-C₆ alkyl, C₁-C₆ alkoxy, substituted C₁-C₆ alkoxy, protected amino or substituted amino; and
   R_{b} is O or S. In certain embodiments, R_{b} is O and Rₐ and R_{c} are each, independently, OCH₃, OCH₂CH₃ or CH(CH₃)₂.

In certain embodiments, G is halogen, OCH₃, OCH₂F, OCHF₂, OCF₃, OCH₂CH₃, O(CH₂)₂F, OCH₂CHF₂, OCH₂CF₃, OCH₂—CH═CH₂, O(CH₂)₂—OCH₃, O(CH₂)₂—SCH₃, O(CH₂)₂—OCF₃, O(CH₂)₃—N(R₁₀)(R₁), O(CH₂)₂—ON(R₁₀)(R₁), O(CH₂)₂—O(CH₂)₂—N(R₁₀)(R₁), OCH₂C(═O)—N(R₁₀)(R₁), OCH₂C(═O)—N(R₁₂)—(CH₂)₂—N(R₁₀)(R₁) or O(CH₂)₂—N(R₁₂)—C(═NR₁₃)[N(R₁₀)(R₁₁)] wherein R₁₀, R₁₁, R₁₂ and R₁₃ are each, independently, H or C₁-C₆ alkyl. In certain embodiments, G is halogen, OCH₃, OCF₃, OCH₂CH₃, OCH₂CF₃, OCH₂—CH═CH₂, O(CH₂)₂—OCH₃, O(CH₂)₂—O(CH₂)₂—N(CH₃)₂, OCH₂C(═O)—N(H)CH₃, OCH₂C(═O)—N(H)—(CH₂)₂—N(CH₃)₂ or OCH₂—N(H)—C(═NH)NH₂. In certain embodiments, G is F, OCH₃ or O(CH₂)₂—OCH₃. In certain embodiments, G is O(CH₂)₂—OCH₃.

In certain embodiments, the 5-terminal nucleoside has Formula IIe:

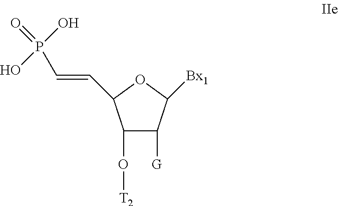

In certain embodiments, antisense compounds, including those particularly suitable for ssRNA comprise one or more type of modified sugar moieties and/or naturally occurring sugar moieties arranged along an oligonucleotide or region thereof in a defined pattern or sugar modification motif. Such motifs may include any of the sugar modifications discussed herein and/or other known sugar modifications.

In certain embodiments, the oligonucleotides comprise or consist of a region having uniform sugar modifications. In certain such embodiments, each nucleoside of the region comprises the same RNA-like sugar modification. In certain embodiments, each nucleoside of the region is a 2′-F nucleoside. In certain embodiments, each nucleoside of the region is a 2′-OMe nucleoside. In certain embodiments, each nucleoside of the region is a 2′-MOE nucleoside. In certain embodiments, each nucleoside of the region is a cEt nucleoside. In certain embodiments, each nucleoside of the region is an LNA nucleoside. In certain embodiments, the uniform region constitutes all or essentially all of the oligonucleotide. In certain embodiments, the region constitutes the entire oligonucleotide except for 1-4 terminal nucleosides.

In certain embodiments, oligonucleotides comprise one or more regions of alternating sugar modifications, wherein the nucleosides alternate between nucleotides having a sugar modification of a first type and nucleotides having a sugar modification of a second type. In certain embodiments, nucleosides of both types are RNA-like nucleosides. In certain embodiments the alternating nucleosides are selected from: 2′-OMe, 2′-F, 2′-MOE, LNA, and cEt. In certain embodiments, the alternating modifications are 2′-F and 2′—OMe. Such regions may be contiguous or may be interrupted by differently modified nucleosides or conjugated nucleosides.

In certain embodiments, the alternating region of alternating modifications each consist of a single nucleoside (i.e., the pattern is (AB)ₓA_{y} wherein A is a nucleoside having a sugar modification of a first type and B is a nucleoside having a sugar modification of a second type; x is 1-20 and y is 0 or 1). In certain embodiments, one or more alternating regions in an alternating motif includes more than a single nucleoside of a type. For example, oligonucleotides may include one or more regions of any of the following nucleoside motifs:
AABBAA;
ABBABB;
AABAAB;
ABBABAABB;
ABABAA;
AABABAB;
ABABAA;
ABBAABBABABAA;
BABBAABBABABAA; or
ABABBAABBABABAA;
   wherein A is a nucleoside of a first type and B is a nucleoside of a second type. In certain embodiments, A and B are each selected from 2′-F, 2′-OMe, BNA, and MOE.

In certain embodiments, oligonucleotides having such an alternating motif also comprise a modified 5′ terminal nucleoside, such as those of formula IIc or IIe.

In certain embodiments, oligonucleotides comprise a region having a 2-2-3 motif Such regions comprises the following motif:

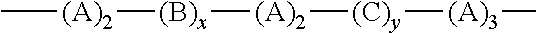

wherein: A is a first type of modified nucleoside;
B and C, are nucleosides that are differently modified than A, however, B and C may have the same or different modifications as one another;
x and y are from 1 to 15.

In certain embodiments, A is a 2′-OMe modified nucleoside. In certain embodiments, B and C are both 2′-F modified nucleosides. In certain embodiments, A is a 2′-OMe modified nucleoside and B and C are both 2′-F modified nucleosides.

In certain embodiments, oligonucleosides have the following sugar motif:

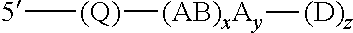

wherein:
   Q is a nucleoside comprising a stabilized phosphate moiety. In certain embodiments, Q is a nucleoside having Formula IIc or IIe;
   A is a first type of modified nucleoside;
   B is a second type of modified nucleoside;
   D is a modified nucleoside comprising a modification different from the nucleoside adjacent to it. Thus, if y is 0, then D must be differently modified than B and if y is 1, then D must be differently modified than A. In certain embodiments, D differs from both A and B.
   X is 5-15;
   Y is 0 or 1;
   Z is 0-4.

In certain embodiments, oligonucleosides have the following sugar motif:

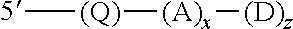

wherein:
   Q is a nucleoside comprising a stabilized phosphate moiety. In certain embodiments, Q is a nucleoside having Formula IIc or IIe;
   A is a first type of modified nucleoside;
   D is a modified nucleoside comprising a modification different from A.
   X is 11-30;
   Z is 0-4.

In certain embodiments A, B, C, and D in the above motifs are selected from: 2′-OMe, 2′-F, 2′-MOE, LNA, and cEt. In certain embodiments, D represents terminal nucleosides. In certain embodiments, such terminal nucleosides are not designed to hybridize to the target nucleic acid (though one or more might hybridize by chance). In certain embodiments, the nucleobase of each D nucleoside is adenine, regardless of the identity of the nucleobase at the corresponding position of the target nucleic acid. In certain embodiments the nucleobase of each D nucleoside is thymine.

In certain embodiments, antisense compounds, including those particularly suited for use as ssRNA comprise modified internucleoside linkages arranged along the oligonucleotide or region thereof in a defined pattern or modified internucleoside linkage motif. In certain embodiments, oligonucleotides comprise a region having an alternating internucleoside linkage motif. In certain embodiments, oligonucleotides comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate internucleoside linkages. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least one block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

Oligonucleotides having any of the various sugar motifs described herein, may have any linkage motif. For example, the oligonucleotides, including but not limited to those described above, may have a linkage motif selected from non-limiting the table below:

| 5′ most | | |
|---|---|---|
| linkage | Central region | 3′-region |
| PS | Alternating PO/PS | 6 PS |
| PS | Alternating PO/PS | 7 PS |
| PS | Alternating PO/PS | 8 PS |

ii. siRNA Compounds

In certain embodiments, antisense compounds are double-stranded RNAi compounds (siRNA). In such embodiments, one or both strands may comprise any modification motif described above for ssRNA. In certain embodiments, ssRNA compounds may be unmodified RNA. In certain embodiments, siRNA compounds may comprise unmodified RNA nucleosides, but modified internucleoside linkages.

Several embodiments relate to double-stranded compositions wherein each strand comprises a motif defined by the location of one or more modified or unmodified nucleosides. In certain embodiments, compositions are provided comprising a first and a second oligomeric compound that are fully or at least partially hybridized to form a duplex region and further comprising a region that is complementary to and hybridizes to a nucleic acid target. It is suitable that such a composition comprise a first oligomeric compound that is an antisense strand having full or partial complementarity to a nucleic acid target and a second oligomeric compound that is a sense strand having one or more regions of complementarity to and forming at least one duplex region with the first oligomeric compound.

The compositions of several embodiments modulate gene expression by hybridizing to a nucleic acid target resulting in loss of its normal function. In some embodiments, the target nucleic acid is CFB. In certain embodiment, the degradation of the targeted CFB is facilitated by an activated RISC complex that is formed with compositions of the invention.

Several embodiments are directed to double-stranded compositions wherein one of the strands is useful in, for example, influencing the preferential loading of the opposite strand into the RISC (or cleavage) complex. The compositions are useful for targeting selected nucleic acid molecules and modulating the expression of one or more genes. In some embodiments, the compositions of the present invention hybridize to a portion of a target RNA resulting in loss of normal function of the target RNA.

Certain embodiments are drawn to double-stranded compositions wherein both the strands comprises a hemimer motif, a fully modified motif, a positionally modified motif or an alternating motif. Each strand of the compositions of the present invention can be modified to fulfil a particular role in for example the siRNA pathway. Using a different motif in each strand or the same motif with different chemical modifications in each strand permits targeting the antisense strand for the RISC complex while inhibiting the incorporation of the sense strand. Within this model, each strand can be independently modified such that it is enhanced for its particular role. The antisense strand can be modified at the 5′-end to enhance its role in one region of the RISC while the 3′-end can be modified differentially to enhance its role in a different region of the RISC.

The double-stranded oligonucleotide molecules can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The double-stranded oligonucleotide molecules can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double-stranded structure, for example wherein the double-stranded region is about 15 to about 30, e.g., about 15, 16, 17, 18, 19, 20, 21, 22, 23, 24, 25, 26, 27, 28, 29 or 30 base pairs; the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof (e.g., about 15 to about 25 or more nucleotides of the double-stranded oligonucleotide molecule are complementary to the target nucleic acid or a portion thereof). Alternatively, the double-stranded oligonucleotide is assembled from a single oligonucleotide, where the self-complementary sense and antisense regions of the siRNA are linked by means of a nucleic acid based or non-nucleic acid-based linker(s).

The double-stranded oligonucleotide can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The double-stranded oligonucleotide can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siRNA molecule capable of mediating RNAi.

In certain embodiments, the double-stranded oligonucleotide comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the double-stranded oligonucleotide comprises nucleotide sequence that is complementary to nucleotide sequence of a target gene. In another embodiment, the double-stranded oligonucleotide interacts with nucleotide sequence of a target gene in a manner that causes inhibition of expression of the target gene.

As used herein, double-stranded oligonucleotides need not be limited to those molecules containing only RNA, but further encompasses chemically modified nucleotides and non-nucleotides. In certain embodiments, the short interfering nucleic acid molecules lack 2′-hydroxy (2′-OH) containing nucleotides. In certain embodiments short interfering nucleic acids optionally do not include any ribonucleotides (e.g., nucleotides having a 2′—OH group). Such double-stranded oligonucleotides that do not require the presence of ribonucleotides within the molecule to support RNAi can however have an attached linker or linkers or other attached or associated groups, moieties, or chains containing one or more nucleotides with 2′—OH groups. Optionally, double-stranded oligonucleotides can comprise ribonucleotides at about 5, 10, 20, 30, 40, or 50% of the nucleotide positions. As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics. For example, double-stranded oligonucleotides can be used to epigenetically silence genes at both the post-transcriptional level and the pre-transcriptional level. In a non-limiting example, epigenetic regulation of gene expression by siRNA molecules of the invention can result from siRNA mediated modification of chromatin structure or methylation pattern to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

It is contemplated that compounds and compositions of several embodiments provided herein can target CFB by a dsRNA-mediated gene silencing or RNAi mechanism, including, e.g., “hairpin” or stem-loop double-stranded RNA effector molecules in which a single RNA strand with self-complementary sequences is capable of assuming a double-stranded conformation, or duplex dsRNA effector molecules comprising two separate strands of RNA. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999. The dsRNA or dsRNA effector molecule may be a single molecule with a region of self-complementarity such that nucleotides in one segment of the molecule base pair with nucleotides in another segment of the molecule. In various embodiments, a dsRNA that consists of a single molecule consists entirely of ribonucleotides or includes a region of ribonucleotides that is complementary to a region of deoxyribonucleotides. Alternatively, the dsRNA may include two different strands that have a region of complementarity to each other.

In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides. In certain embodiments, the regions of complementarity are at least 70, 80, 90, 95, 98, or 100% complementary to each other and to a target nucleic acid sequence. In certain embodiments, the region of the dsRNA that is present in a double-stranded conformation includes at least 19, 20, 21, 22, 23, 24, 25, 26, 27, 28, 29, 30, 50, 75, 100, 200, 500, 1000, 2000 or 5000 nucleotides or includes all of the nucleotides in a cDNA or other target nucleic acid sequence being represented in the dsRNA. In some embodiments, the dsRNA does not contain any single stranded regions, such as single stranded ends, or the dsRNA is a hairpin. In other embodiments, the dsRNA has one or more single stranded regions or overhangs. In certain embodiments, RNA/DNA hybrids include a DNA strand or region that is an antisense strand or region (e.g, has at least 70, 80, 90, 95, 98, or 100% complementarity to a target nucleic acid) and an RNA strand or region that is a sense strand or region (e.g, has at least 70, 80, 90, 95, 98, or 100% identity to a target nucleic acid), and vice versa.

In various embodiments, the RNA/DNA hybrid is made in vitro using enzymatic or chemical synthetic methods such as those described herein or those described in WO 00/63364, filed Apr. 19, 2000, or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell. In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000, or U.S. Ser. No. 60/130,377, filed Apr. 21, 1999.) Exemplary circular nucleic acids include lariat structures in which the free 5′ phosphoryl group of a nucleotide becomes linked to the 2′ hydroxyl group of another nucleotide in a loop back fashion.

In other embodiments, the dsRNA includes one or more modified nucleotides in which the 2′ position in the sugar contains a halogen (such as fluorine group) or contains an alkoxy group (such as a methoxy group) which increases the half-life of the dsRNA in vitro or in vivo compared to the corresponding dsRNA in which the corresponding 2′ position contains a hydrogen or an hydroxyl group. In yet other embodiments, the dsRNA includes one or more linkages between adjacent nucleotides other than a naturally-occurring phosphodiester linkage. Examples of such linkages include phosphoramide, phosphorothioate, and phosphorodithioate linkages. The dsRNAs may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000, or U.S. Ser. No. 60/130,377, filed Apr. 21, 1999.

In other embodiments, the dsRNA can be any of the at least partially dsRNA molecules disclosed in WO 00/63364, as well as any of the dsRNA molecules described in U.S. Provisional Application 60/399,998; and U.S. Provisional Application 60/419,532, and PCT/US2003/033466, the teaching of which is hereby incorporated by reference. Any of the dsRNAs may be expressed in vitro or in vivo using the methods described herein or standard methods, such as those described in WO 00/63364.

### Occupancy

In certain embodiments, antisense compounds are not expected to result in cleavage or the target nucleic acid via RNase H or to result in cleavage or sequestration through the RISC pathway. In certain such embodiments, antisense activity may result from occupancy, wherein the presence of the hybridized antisense compound disrupts the activity of the target nucleic acid. In certain such embodiments, the antisense compound may be uniformly modified or may comprise a mix of modifications and/or modified and unmodified nucleosides.

### Target Nucleic Acids, Target Regions and Nucleotide Sequences

Nucleotide sequences that encode Complement Factor B (CFB) include, without limitation, the following: GENBANK Accession No. NM_001710.5 (incorporated herein as SEQ ID NO: 1), GENBANK Accession No. NT_007592.15 truncated from nucleotides 31852000 to 31861000 (incorporated herein as SEQ ID NO: 2), GENBANK Accession No NW_001116486.1 truncated from nucleotides 536000 to 545000 (incorporated herein as SEQ ID NO: 3), GENBANK Accession No. XM_001113553.2 (incorporated herein as SEQ ID NO: 4), or GENBANK Accession No. NM_008198.2 (incorporated herein as SEQ ID NO: 5).

### Hybridization

In some embodiments, hybridization occurs between an antisense compound disclosed herein and a CFB nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a CFB nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a CFB nucleic acid).

Non-complementary nucleobases between an antisense compound and a CFB nucleic acid may be tolerated provided that the antisense compound remains able to specifically hybridize to a target nucleic acid. Moreover, an antisense compound may hybridize over one or more segments of a CFB nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a CFB nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having four noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e. 100% complementary) to a target nucleic acid, or specified portion thereof. For example, an antisense compound may be fully complementary to a CFB nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e. linked) or non-contiguous. In one embodiment, a non-complementary nucleobase is located in the wing segment of a gapmer antisense oligonucleotide.

In certain embodiments, antisense compounds that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a CFB nucleic acid, or specified portion thereof.

In certain embodiments, antisense compounds that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a CFB nucleic acid, or specified portion thereof.

The antisense compounds provided also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds are complementary to at least an 11 nucleobase portion of a target segment. In certain embodiments, the antisense compounds are complementary to at least a 12 nucleobase portion of a target segment. In certain embodiments, the antisense compounds are complementary to at least a 13 nucleobase portion of a target segment. In certain embodiments, the antisense compounds are complementary to at least a 14 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases maybe adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occurring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a CFB nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosponate linkages. In certain embodiments, oligonucleotides having a gapmer nucleoside motif comprise a linkage motif comprising all phosphorothioate linkages except for one or two methylphosponate linkages. In certain embodiments, one methylphosponate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′—OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each Rₗ, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising abridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′—(CH₂)₂-0-2′ (ENA); 4′-CH(CH₃)—O-2′ (also referred to as constrained ethyl or cEt) and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO 2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Zhou et al., J. Org. Chem., 2009, 74, 118-134); and 4′-CH₂—C(═CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J Org. Chem., 1998, 63, 10035-10039; Srivastava et al., J Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; 8,530,640; and 7,696,345; U.S. Patent Publication No. US2008-0039618; US2009-0012281; U.S. Patent Serial Nos. 61/026,995 and 61/097,787; Published PCT International applications; WO 2009/067647; WO 2011/017521; WO 2010/036698 WO 1999/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ, —[C(Rₐ)(R_{b})]ₙO—, —C(RₐR_{b})—N(R)—O— or —C(RₐR_{b})—O—N(R)—. In certain embodiments, the bridge is 4′-CH₂-2′, 4′—(CH₂)₂-2′, 4′—(CH₂)₃-2′, 4′—CH₂—O-2′, 4′—(CH₂)₂—O-2′, 4′—CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA and (K) vinyl BNA as depicted below:

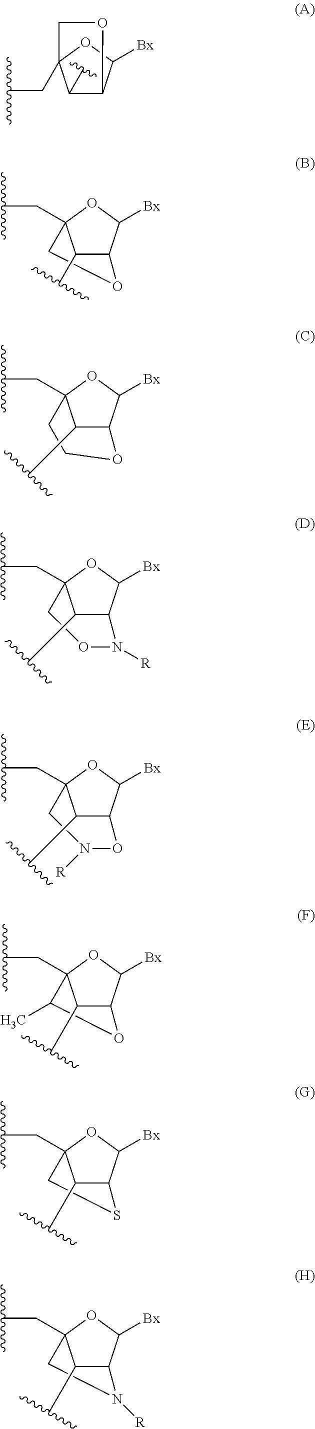

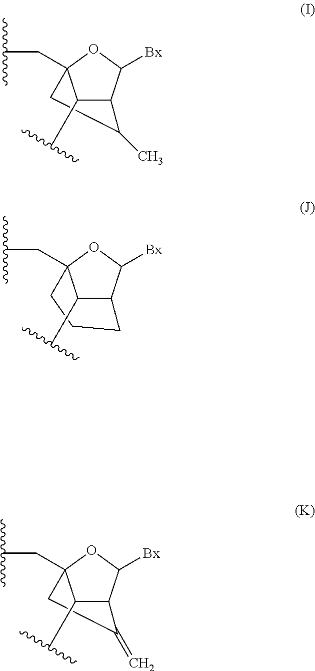

wherein Bx is the base moiety and R is independently H, a protecting group, C₁-C₁₂ alkyl or C₁-C₁₂ alkoxy.

In certain embodiments, bicyclic nucleosides are provided having Formula I:

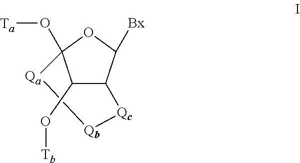

wherein:
   Bx is a heterocyclic base moiety;
   Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;
   R_{c} is C₁-C₁₂ alkyl or an amino protecting group; and
   Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

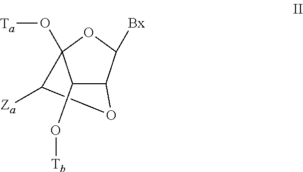

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}Ja, wherein each J_{c}, J_{d} and J_{c} is, independently, H, C₁-C₆ alkyl, or substituted C₁-C₆ alkyl and X is O or NJ_{c}.

In certain embodiments, bicyclic nucleosides are provided having Formula III:

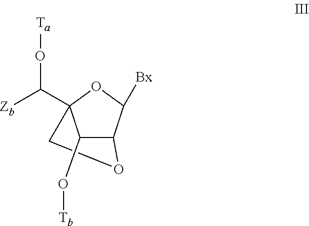

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

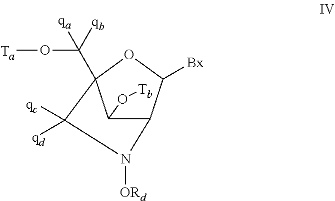

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;
   each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

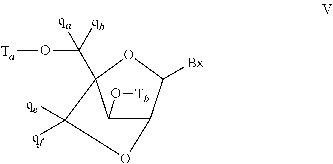

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;
   or qₑ and q_{f} together are ═C(q_{g})(qₕ);
   q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel conformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

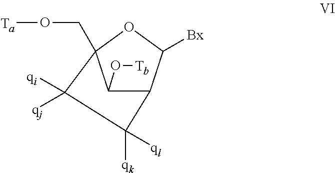

wherein:
   Bx is a heterocyclic base moiety;
   Tₐ and T_{b} are each, independently H, a hydroxyl protecting group, a conjugate group, a reactive phosphorus group, a phosphorus moiety or a covalent attachment to a support medium;
   each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and
   qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′—O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854) or fluoro HNA (F-HNA) having a tetrahydropyran ring system as illustrated below:

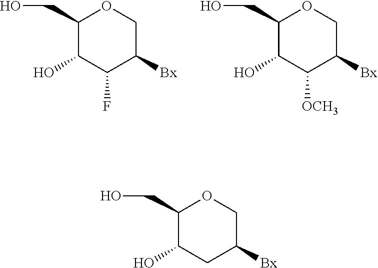

In certain embodiments, sugar surrogates are selected having Formula VII:

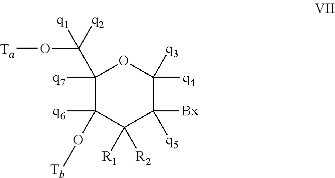

wherein independently for each of said at least one tetrahydropyran nucleoside analog of Formula VII:
   Bx is a heterocyclic base moiety;
   Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;
   q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇ is other than H.

In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is methoxyethoxy and R₂ is H.

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following formula:

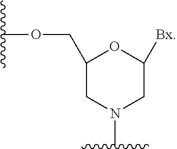

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 published on Aug. 21, 2008 for other disclosed 5′, 2′-bis substituted nucleosides) and replacement of the ribosyl ring oxygen atom with S and further substitution at the 2′-position (see published U.S. Patent Application US2005-0130923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc. 2007, 129(26), 8362-8379).

In certain embodiments, antisense compounds comprise one or more modified cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J Am. Chem. Soc., 2008, 130(6), 1979-1984; Horváth et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-998; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCT application, WO 06/047842; and Published PCT Application WO 01/049687; the text of each is incorporated by reference herein, in their entirety). Certain modified cyclohexenyl nucleosides have Formula X.

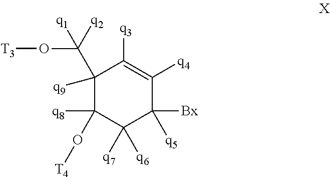

wherein independently for each of said at least one cyclohexenyl nucleoside analog of Formula X:
Bx is a heterocyclic base moiety;
T₃ and T₄ are each, independently, an internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and
q₁, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, 0-allyl, O—C₁-C₁₀ alkyl, —OCF₃, O—(CH₂)₂O—CH₃, 2′—O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position of the sugar ring.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854). Such ring systems can undergo various additional substitutions to enhance activity.

Methods for the preparations of modified sugars are well known to those skilled in the art. Some representative U.S. patents that teach the preparation of such modified sugars include without limitation, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to antisense compounds. Modified nucleobases include synthetic and natural nucleobases such as, for example, 5-methylcytosine (5-me-C). Certain nucleobase substitutions, including 5-methylcytosine substitutions, are particularly useful for increasing the binding affinity of an antisense compound for a target nucleic acid. For example, 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278).

Additional modified nucleobases include 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties can also include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense compounds include 5-substituted pyrimidines, 6-azapyrimidines and N-2, N-6 and 0-6 substituted purines, including 2 aminopropyladenine, 5-propynyluracil and 5-propynylcytosine.

In certain embodiments, antisense compounds targeted to a CFB nucleic acid comprise one or more modified nucleobases. In certain embodiments, shortened or gap-widened antisense oligonucleotides targeted to a CFB nucleic acid comprise one or more modified nucleobases. In certain embodiments, the modified nucleobase is 5-methylcytosine. In certain embodiments, each cytosine is a 5-methylcytosine.

### Conjugated Antisense Compounds

In certain embodiments, the present disclosure provides conjugated antisense compounds. In certain embodiments, the present disclosure provides conjugated antisense compounds comprising an antisense oligonucleotide complementary to a nucleic acid transcript. In certain embodiments, the present disclosure provides methods comprising contacting a cell with a conjugated antisense compound comprising an antisense oligonucleotide complementary to a nucleic acid transcript. In certain embodiments, the present disclosure provides methods comprising contacting a cell with a conjugated antisense compound comprising an antisense oligonucleotide and reducing the amount or activity of a nucleic acid transcript in a cell.

The asialoglycoprotein receptor (ASGP-R) has been described previously. See e.g., Park et al., PNAS vol. 102, No. 47, pp 17125-17129 (2005). Such receptors are expressed on liver cells, particularly hepatocytes. Further, it has been shown that compounds comprising clusters of three N-acetylgalactosamine (GalNAc) ligands are capable of binding to the ASGP-R, resulting in uptake of the compound into the cell. See e.g., Khorev et al., Bioorganic and Medicinal Chemistry, 16, 9, pp 5216-5231 (May 2008). Accordingly, conjugates comprising such GalNAc clusters have been used to facilitate uptake of certain compounds into liver cells, specifically hepatocytes. For example it has been shown that certain GalNAc-containing conjugates increase activity of duplex siRNA compounds in liver cells in vivo. In such instances, the GalNAc-containing conjugate is typically attached to the sense strand of the siRNA duplex. Since the sense strand is discarded before the antisense strand ultimately hybridizes with the target nucleic acid, there is little concern that the conjugate will interfere with activity. Typically, the conjugate is attached to the 3′ end of the sense strand of the siRNA. See e.g., U.S. Pat. No. 8,106,022. Certain conjugate groups described herein are more active and/or easier to synthesize than conjugate groups previously described.

In certain embodiments of the present invention, conjugates are attached to single-stranded antisense compounds, including, but not limited to RNase H based antisense compounds and antisense compounds that alter splicing of a pre-mRNA target nucleic acid. In such embodiments, the conjugate should remain attached to the antisense compound long enough to provide benefit (improved uptake into cells) but then should either be cleaved, or otherwise not interfere with the subsequent steps necessary for activity, such as hybridization to a target nucleic acid and interaction with RNase H or enzymes associated with splicing or splice modulation. This balance of properties is more important in the setting of single-stranded antisense compounds than in siRNA compounds, where the conjugate may simply be attached to the sense strand. Disclosed herein are conjugated single-stranded antisense compounds having improved potency in liver cells in vivo compared with the same antisense compound lacking the conjugate. Given the required balance of properties for these compounds such improved potency is surprising.

In certain embodiments, conjugate groups herein comprise a cleavable moiety. As noted, without wishing to be bound by mechanism, it is logical that the conjugate should remain on the compound long enough to provide enhancement in uptake, but after that, it is desirable for some portion or, ideally, all of the conjugate to be cleaved, releasing the parent compound (e.g., antisense compound) in its most active form. In certain embodiments, the cleavable moiety is a cleavable nucleoside. Such embodiments take advantage of endogenous nucleases in the cell by attaching the rest of the conjugate (the cluster) to the antisense oligonucleotide through a nucleoside via one or more cleavable bonds, such as those of a phosphodiester linkage. In certain embodiments, the cluster is bound to the cleavable nucleoside through a phosphodiester linkage. In certain embodiments, the cleavable nucleoside is attached to the antisense oligonucleotide (antisense compound) by a phosphodiester linkage. In certain embodiments, the conjugate group may comprise two or three cleavable nucleosides. In such embodiments, such cleavable nucleosides are linked to one another, to the antisense compound and/or to the cluster via cleavable bonds (such as those of a phosphodiester linkage). Certain conjugates herein do not comprise a cleavable nucleoside and instead comprise a cleavable bond. It is shown that that sufficient cleavage of the conjugate from the oligonucleotide is provided by at least one bond that is vulnerable to cleavage in the cell (a cleavable bond).

In certain embodiments, conjugated antisense compounds are prodrugs. Such prodrugs are administered to an animal and are ultimately metabolized to a more active form. For example, conjugated antisense compounds are cleaved to remove all or part of the conjugate resulting in the active (or more active) form of the antisense compound lacking all or some of the conjugate.

In certain embodiments, conjugates are attached at the 5′ end of an oligonucleotide. Certain such 5′-conjugates are cleaved more efficiently than counterparts having a similar conjugate group attached at the 3′ end. In certain embodiments, improved activity may correlate with improved cleavage. In certain embodiments, oligonucleotides comprising a conjugate at the 5′ end have greater efficacy than oligonucleotides comprising a conjugate at the 3′ end (see, for example, Examples 56, 81, 83, and 84). Further, 5′-attachment allows simpler oligonucleotide synthesis. Typically, oligonucleotides are synthesized on a solid support in the 3′ to 5′ direction. To make a 3′-conjugated oligonucleotide, typically one attaches a pre-conjugated 3′ nucleoside to the solid support and then builds the oligonucleotide as usual. However, attaching that conjugated nucleoside to the solid support adds complication to the synthesis. Further, using that approach, the conjugate is then present throughout the synthesis of the oligonucleotide and can become degraded during subsequent steps or may limit the sorts of reactions and reagents that can be used. Using the structures and techniques described herein for 5′-conjugated oligonucleotides, one can synthesize the oligonucleotide using standard automated techniques and introduce the conjugate with the final (5′-most) nucleoside or after the oligonucleotide has been cleaved from the solid support.

In view of the art and the present disclosure, one of ordinary skill can easily make any of the conjugates and conjugated oligonucleotides herein. Moreover, synthesis of certain such conjugates and conjugated oligonucleotides disclosed herein is easier and/or requires few steps, and is therefore less expensive than that of conjugates previously disclosed, providing advantages in manufacturing. For example, the synthesis of certain conjugate groups consists of fewer synthetic steps, resulting in increased yield, relative to conjugate groups previously described. Conjugate groups such as GalNAc3-10 in Example 46 and GalNAc3-7 in Example 48 are much simpler than previously described conjugates such as those described in U.S. Pat. No. 8,106,022 or U.S. Pat. No. 7,262,177 that require assembly of more chemical intermediates. Accordingly, these and other conjugates described herein have advantages over previously described compounds for use with any oligonucleotide, including single-stranded oligonucleotides and either strand of double-stranded oligonucleotides (e.g., siRNA).

Similarly, disclosed herein are conjugate groups having only one or two GalNAc ligands. As shown, such conjugates groups improve activity of antisense compounds. Such compounds are much easier to prepare than conjugates comprising three GalNAc ligands. Conjugate groups comprising one or two GalNAc ligands may be attached to any antisense compounds, including single-stranded oligonucleotides and either strand of double-stranded oligonucleotides (e.g., siRNA).

In certain embodiments, the conjugates herein do not substantially alter certain measures of tolerability. For example, it is shown herein that conjugated antisense compounds are not more immunogenic than unconjugated parent compounds. Since potency is improved, embodiments in which tolerability remains the same (or indeed even if tolerability worsens only slightly compared to the gains in potency) have improved properties for therapy.

In certain embodiments, conjugation allows one to alter antisense compounds in ways that have less attractive consequences in the absence of conjugation. For example, in certain embodiments, replacing one or more phosphorothioate linkages of a fully phosphorothioate antisense compound with phosphodiester linkages results in improvement in some measures of tolerability. For example, in certain instances, such antisense compounds having one or more phosphodiester are less immunogenic than the same compound in which each linkage is a phosphorothioate. However, in certain instances, as shown in Example 26, that same replacement of one or more phosphorothioate linkages with phosphodiester linkages also results in reduced cellular uptake and/or loss in potency. In certain embodiments, conjugated antisense compounds described herein tolerate such change in linkages with little or no loss in uptake and potency when compared to the conjugated full-phosphorothioate counterpart. In fact, in certain embodiments, for example, in Examples 44, 57, 59, and 86, oligonucleotides comprising a conjugate and at least one phosphodiester internucleoside linkage actually exhibit increased potency in vivo even relative to a full phosphorothioate counterpart also comprising the same conjugate. Moreover, since conjugation results in substantial increases in uptake/potency a small loss in that substantial gain may be acceptable to achieve improved tolerability. Accordingly, in certain embodiments, conjugated antisense compounds comprise at least one phosphodiester linkage.

In certain embodiments, conjugation of antisense compounds herein results in increased delivery, uptake and activity in hepatocytes. Thus, more compound is delivered to liver tissue. However, in certain embodiments, that increased delivery alone does not explain the entire increase in activity. In certain such embodiments, more compound enters hepatocytes. In certain embodiments, even that increased hepatocyte uptake does not explain the entire increase in activity. In such embodiments, productive uptake of the conjugated compound is increased. For example, as shown in Example 102, certain embodiments of GalNAc-containing conjugates increase enrichment of antisense oligonucleotides in hepatocytes versus non-parenchymal cells. This enrichment is beneficial for oligonucleotides that target genes that are expressed in hepatocytes.

In certain embodiments, conjugated antisense compounds herein result in reduced kidney exposure. For example, as shown in Example 20, the concentrations of antisense oligonucleotides comprising certain embodiments of GalNAc-containing conjugates are lower in the kidney than that of antisense oligonucleotides lacking a GalNAc-containing conjugate. This has several beneficial therapeutic implications. For therapeutic indications where activity in the kidney is not sought, exposure to kidney risks kidney toxicity without corresponding benefit. Moreover, high concentration in kidney typically results in loss of compound to the urine resulting in faster clearance. Accordingly for non-kidney targets, kidney accumulation is undesired.

In certain embodiments, the present disclosure provides conjugated antisense compounds represented by the formula:

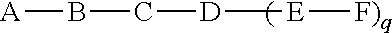

wherein
A is the antisense oligonucleotide;
B is the cleavable moiety
C is the conjugate linker
D is the branching group
each E is a tether;
each F is a ligand; and
q is an integer between 1 and 5.

In the above diagram and in similar diagrams herein, the branching group “D” branches as many times as is necessary to accommodate the number of (E-F) groups as indicated by “q”. Thus, where q=1, the formula is:

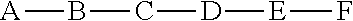

where q=2, the formula is:

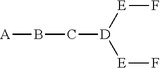

where q=3, the formula is:

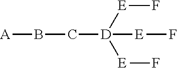

where q=4, the formula is:

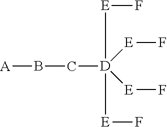

where q=5, the formula is:

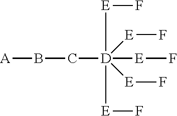

In certain embodiments, conjugated antisense compounds are provided having the structure: Targeting moiety

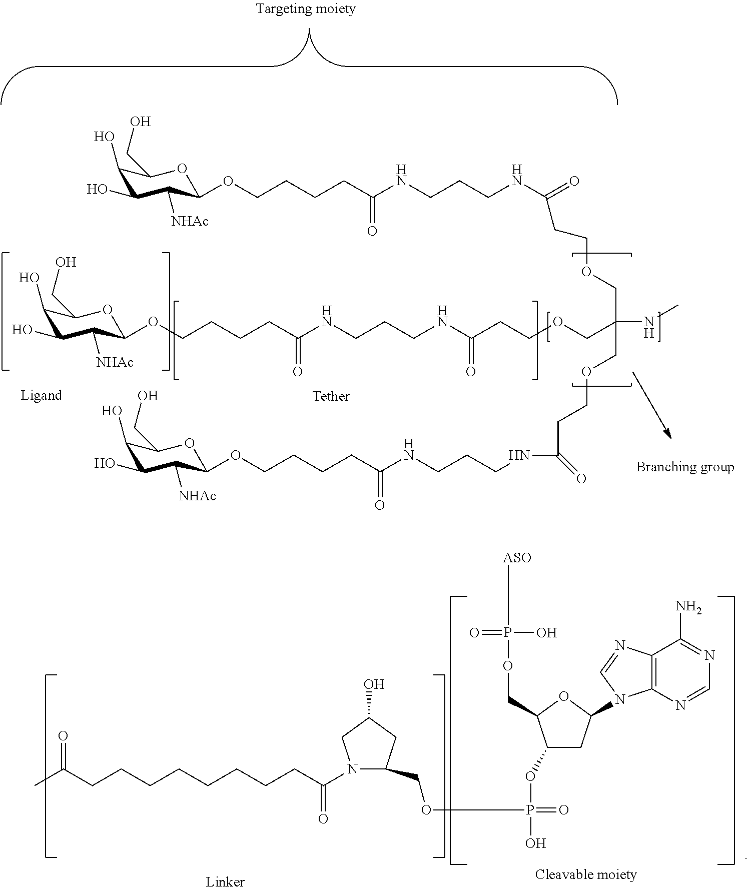

In certain embodiments, conjugated antisense compounds are provided having the structure:

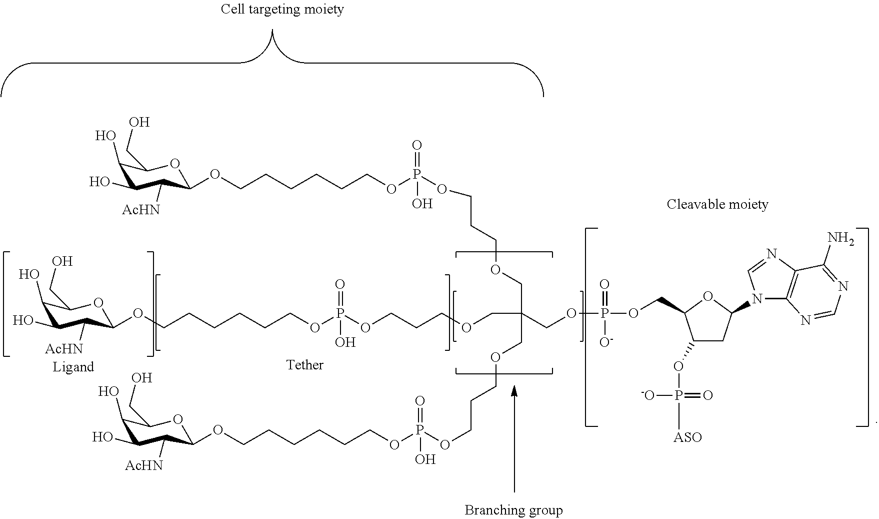

In certain embodiments, conjugated antisense compounds are provided having the structure:

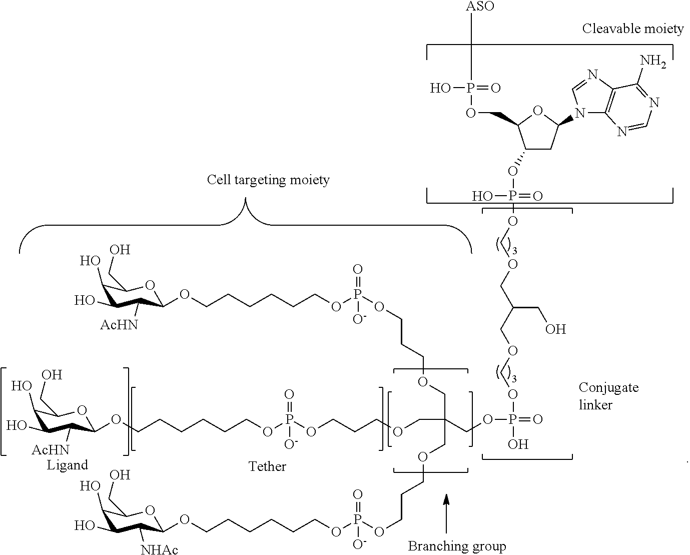

In certain embodiments, conjugated antisense compounds are provided having the structure:

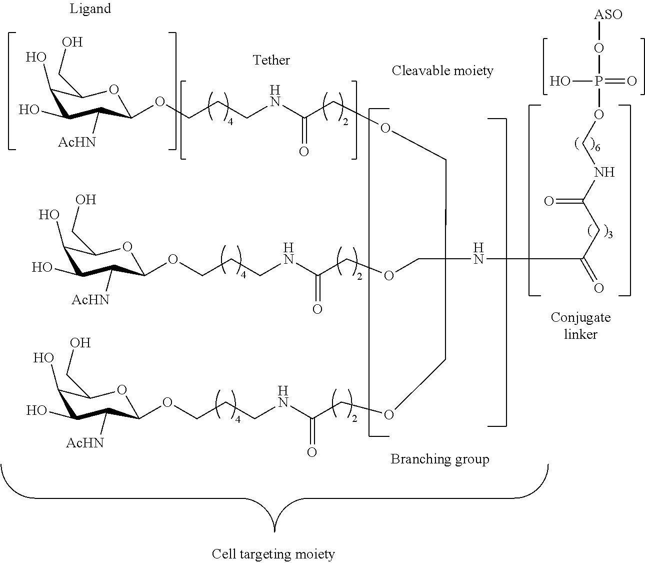

The present disclosure provides the following non-limiting numbered embodiments:
Embodiment 1. The conjugated antisense compound of any of embodiments 1179 to 1182, wherein the tether has a structure selected from among:

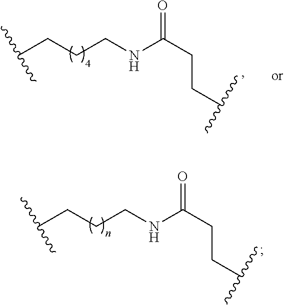

wherein each n is independently, 0, 1, 2, 3, 4, 5, 6, or 7.
Embodiment 2. The conjugated antisense compound of any of embodiments 1179 to 1182, wherein the tether has the structure:

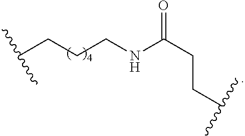

Embodiment 3. The conjugated antisense compound of any of embodiments 1179 to 1182 or 1688 to 1689, wherein the linker has a structure selected from among:

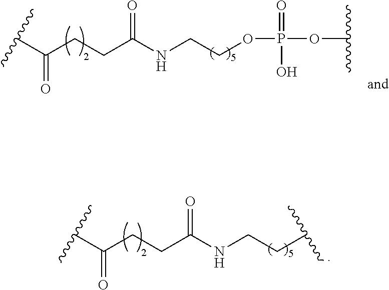

Embodiment 4. The conjugated antisense compound of any of embodiments 1179 to 1182 or 1688 to 1689, wherein the linker has a structure selected from among:

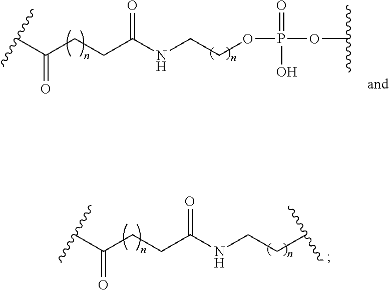

wherein each n is independently, 0, 1, 2, 3, 4, 5, 6, or 7.
Embodiment 5. The conjugated antisense compound of any of embodiments 1179 to 1182 or 1688 to 1689, wherein the linker has the structure:

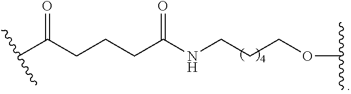

In embodiments having more than one of a particular variable (e.g., more than one “m” or “n”), unless otherwise indicated, each such particular variable is selected independently. Thus, for a structure having more than one n, each n is selected independently, so they may or may not be the same as one another.

i. Certain Cleavable Moieties

In certain embodiments, a cleavable moiety is a cleavable bond. In certain embodiments, a cleavable moiety comprises a cleavable bond. In certain embodiments, the conjugate group comprises a cleavable moiety. In certain such embodiments, the cleavable moiety attaches to the antisense oligonucleotide. In certain such embodiments, the cleavable moiety attaches directly to the cell-targeting moiety. In certain such embodiments, the cleavable moiety attaches to the conjugate linker. In certain embodiments, the cleavable moiety comprises a phosphate or phosphodiester. In certain embodiments, the cleavable moiety is a cleavable nucleoside or nucleoside analog. In certain embodiments, the nucleoside or nucleoside analog comprises an optionally protected heterocyclic base selected from a purine, substituted purine, pyrimidine or substituted pyrimidine. In certain embodiments, the cleavable moiety is a nucleoside comprising an optionally protected heterocyclic base selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. In certain embodiments, the cleavable moiety is 2′-deoxy nucleoside that is attached to the 3′ position of the antisense oligonucleotide by a phosphodiester linkage and is attached to the linker by a phosphodiester or phosphorothioate linkage. In certain embodiments, the cleavable moiety is 2′-deoxy adenosine that is attached to the 3′ position of the antisense oligonucleotide by a phosphodiester linkage and is attached to the linker by a phosphodiester or phosphorothioate linkage. In certain embodiments, the cleavable moiety is 2′-deoxy adenosine that is attached to the 3′ position of the antisense oligonucleotide by a phosphodiester linkage and is attached to the linker by a phosphodiester linkage.

In certain embodiments, the cleavable moiety is attached to the 3′ position of the antisense oligonucleotide. In certain embodiments, the cleavable moiety is attached to the 5′ position of the antisense oligonucleotide. In certain embodiments, the cleavable moiety is attached to a 2′ position of the antisense oligonucleotide. In certain embodiments, the cleavable moiety is attached to the antisense oligonucleotide by a phosphodiester linkage. In certain embodiments, the cleavable moiety is attached to the linker by either a phosphodiester or a phosphorothioate linkage. In certain embodiments, the cleavable moiety is attached to the linker by a phosphodiester linkage. In certain embodiments, the conjugate group does not include a cleavable moiety.

In certain embodiments, the cleavable moiety is cleaved after the complex has been administered to an animal only after being internalized by a targeted cell. Inside the cell the cleavable moiety is cleaved thereby releasing the active antisense oligonucleotide. While not wanting to be bound by theory it is believed that the cleavable moiety is cleaved by one or more nucleases within the cell. In certain embodiments, the one or more nucleases cleave the phosphodiester linkage between the cleavable moiety and the linker. In certain embodiments, the cleavable moiety has a structure selected from among the following:

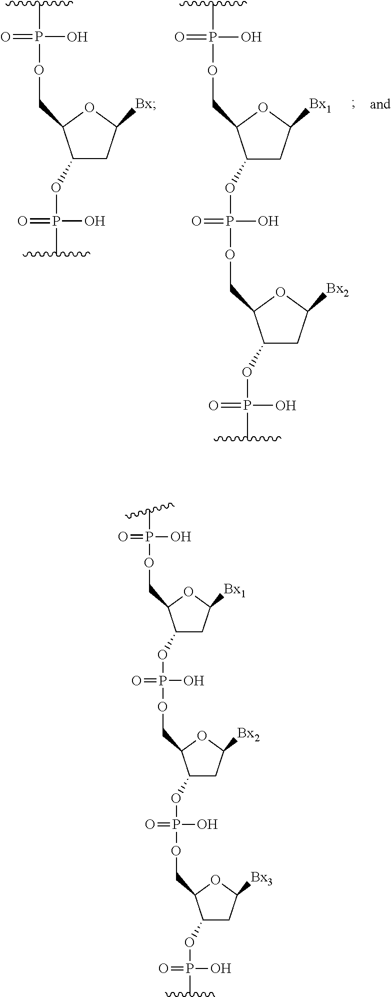

wherein each of Bx, Bx₁, Bx₂, and Bx₃ is independently a heterocyclic base moiety. In certain embodiments, the cleavable moiety has a structure selected from among the following:

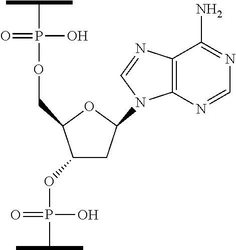

ii. Certain Linkers

In certain embodiments, the conjugate groups comprise a linker. In certain such embodiments, the linker is covalently bound to the cleavable moiety. In certain such embodiments, the linker is covalently bound to the antisense oligonucleotide. In certain embodiments, the linker is covalently bound to a cell-targeting moiety. In certain embodiments, the linker further comprises a covalent attachment to a solid support. In certain embodiments, the linker further comprises a covalent attachment to a protein binding moiety. In certain embodiments, the linker further comprises a covalent attachment to a solid support and further comprises a covalent attachment to a protein binding moiety. In certain embodiments, the linker includes multiple positions for attachment of tethered ligands. In certain embodiments, the linker includes multiple positions for attachment of tethered ligands and is not attached to a branching group. In certain embodiments, the linker further comprises one or more cleavable bond. In certain embodiments, the conjugate group does not include a linker.

In certain embodiments, the linker includes at least a linear group comprising groups selected from alkyl, amide, disulfide, polyethylene glycol, ether, thioether (—S—) and hydroxylamino (—O—N(H)—) groups. In certain embodiments, the linear group comprises groups selected from alkyl, amide and ether groups. In certain embodiments, the linear group comprises groups selected from alkyl and ether groups. In certain embodiments, the linear group comprises at least one phosphorus linking group. In certain embodiments, the linear group comprises at least one phosphodiester group. In certain embodiments, the linear group includes at least one neutral linking group. In certain embodiments, the linear group is covalently attached to the cell-targeting moiety and the cleavable moiety. In certain embodiments, the linear group is covalently attached to the cell-targeting moiety and the antisense oligonucleotide. In certain embodiments, the linear group is covalently attached to the cell-targeting moiety, the cleavable moiety and a solid support. In certain embodiments, the linear group is covalently attached to the cell-targeting moiety, the cleavable moiety, a solid support and a protein binding moiety. In certain embodiments, the linear group includes one or more cleavable bond.

In certain embodiments, the linker includes the linear group covalently attached to a scaffold group. In certain embodiments, the scaffold includes a branched aliphatic group comprising groups selected from alkyl, amide, disulfide, polyethylene glycol, ether, thioether and hydroxylamino groups. In certain embodiments, the scaffold includes a branched aliphatic group comprising groups selected from alkyl, amide and ether groups. In certain embodiments, the scaffold includes at least one mono or polycyclic ring system. In certain embodiments, the scaffold includes at least two mono or polycyclic ring systems. In certain embodiments, the linear group is covalently attached to the scaffold group and the scaffold group is covalently attached to the cleavable moiety and the linker. In certain embodiments, the linear group is covalently attached to the scaffold group and the scaffold group is covalently attached to the cleavable moiety, the linker and a solid support. In certain embodiments, the linear group is covalently attached to the scaffold group and the scaffold group is covalently attached to the cleavable moiety, the linker and a protein binding moiety. In certain embodiments, the linear group is covalently attached to the scaffold group and the scaffold group is covalently attached to the cleavable moiety, the linker, a protein binding moiety and a solid support. In certain embodiments, the scaffold group includes one or more cleavable bond.

In certain embodiments, the linker includes a protein binding moiety. In certain embodiments, the protein binding moiety is a lipid such as for example including but not limited to cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In certain embodiments, the protein binding moiety is a C16 to C22 long chain saturated or unsaturated fatty acid, cholesterol, cholic acid, vitamin E, adamantane or 1-pentafluoropropyl.

In certain embodiments, a linker has a structure selected from among:

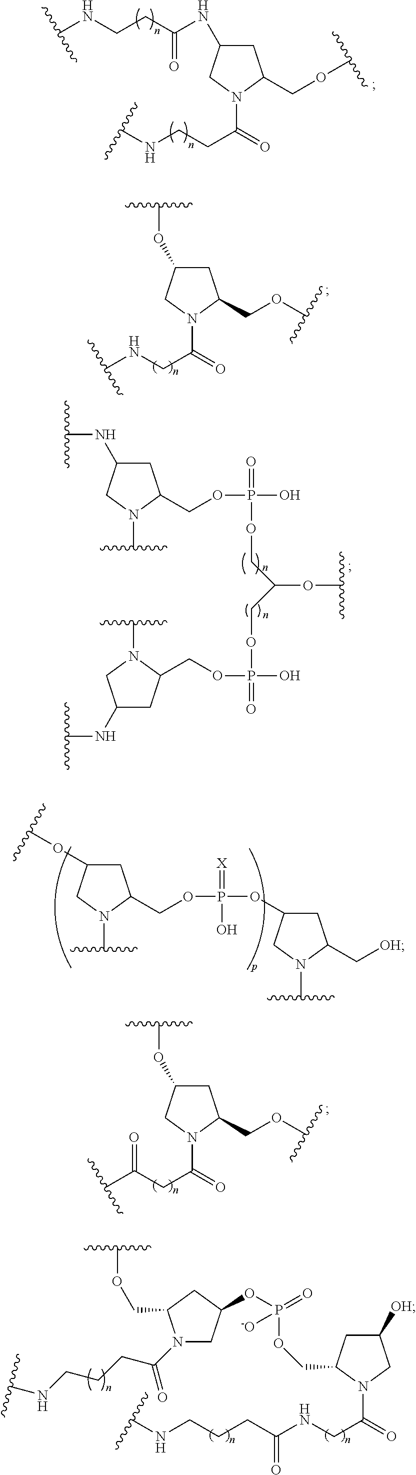

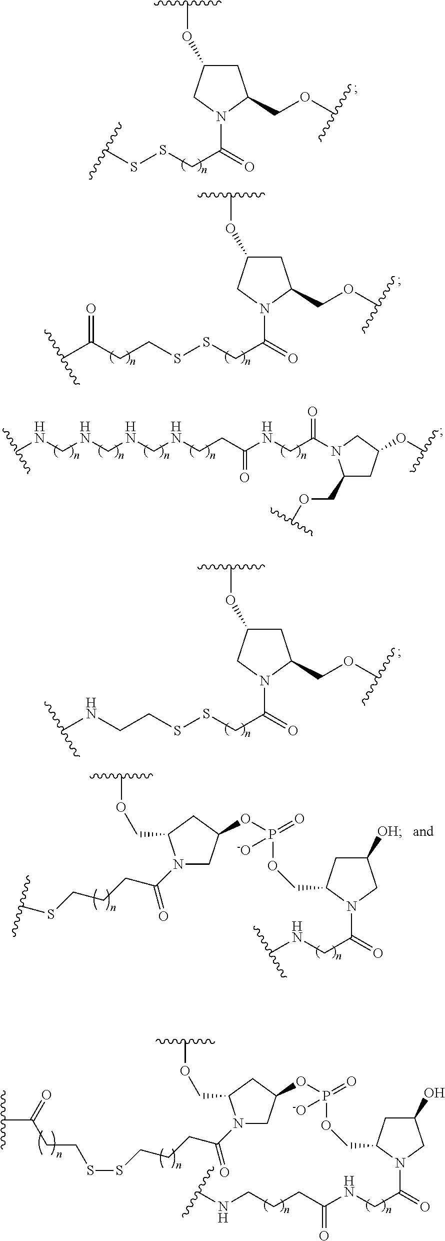

wherein each n is, independently, from 1 to 20; and p is from 1 to 6.

In certain embodiments, a linker has a structure selected from among:

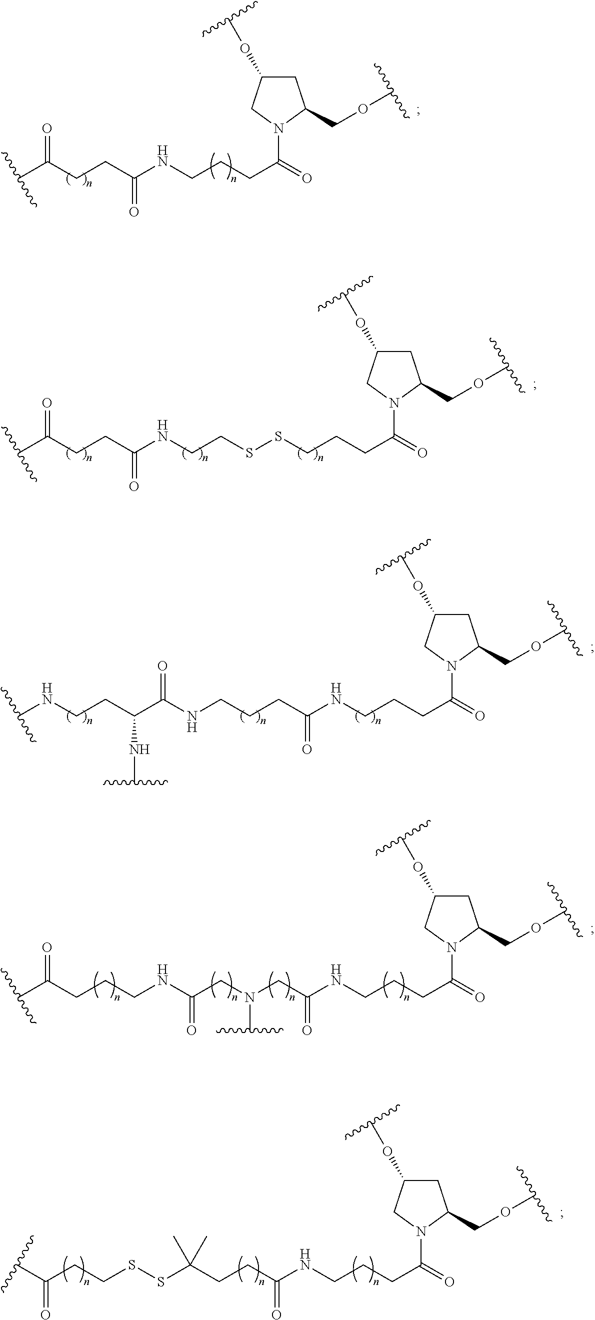

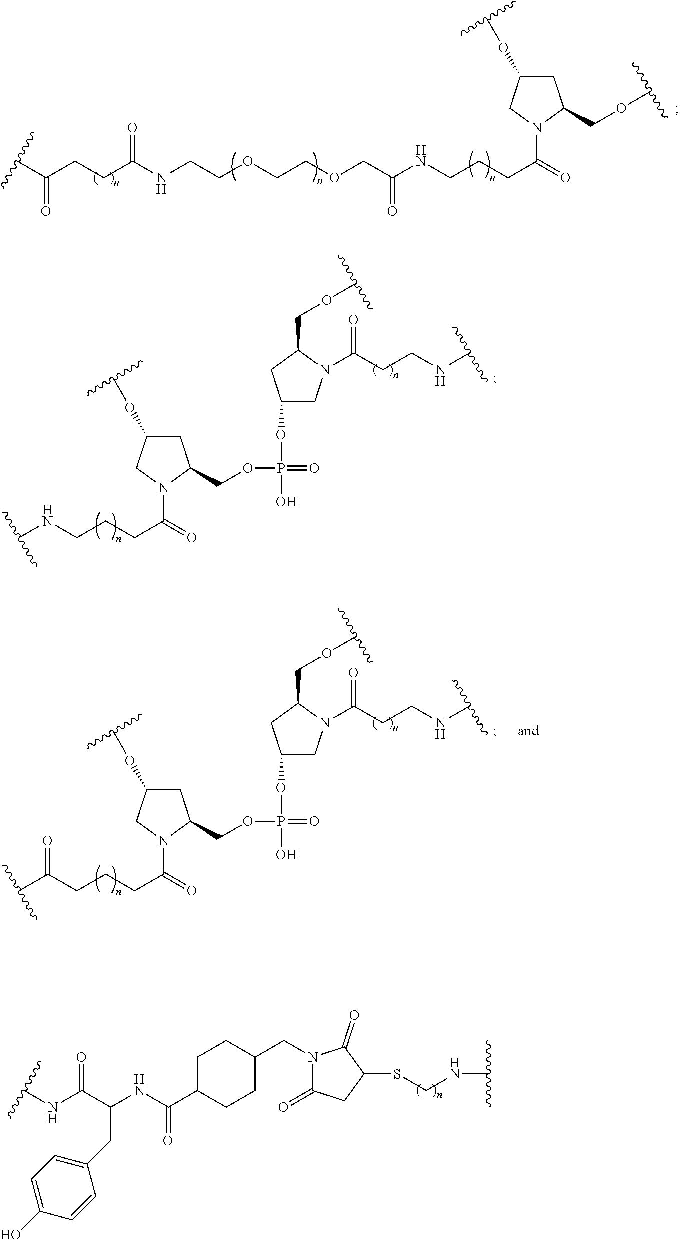

wherein each n is, independently, from 1 to 20.

In certain embodiments, a linker has a structure selected from among:

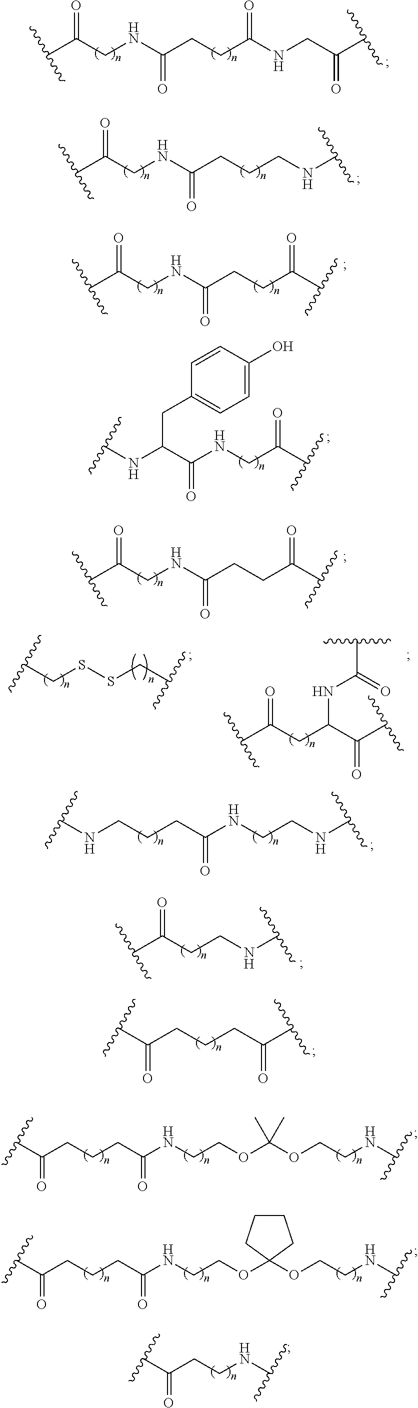

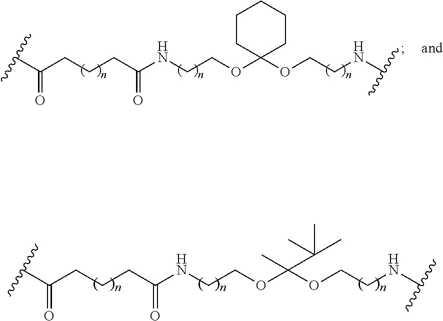

wherein n is from 1 to 20.

In certain embodiments, a linker has a structure selected from among:

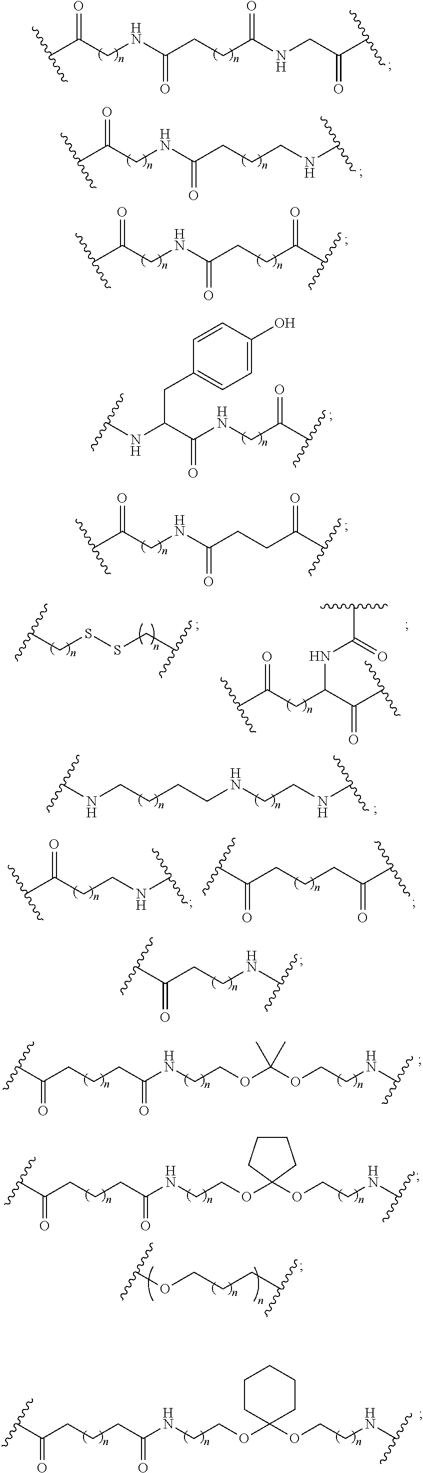

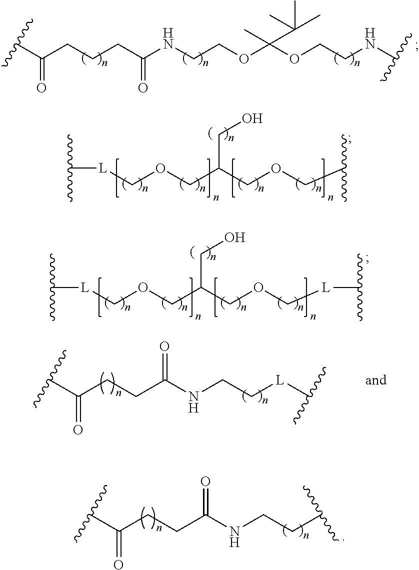

wherein each L is, independently, a phosphorus linking group or a neutral linking group; and each n is, independently, from 1 to 20.

In certain embodiments, a linker has a structure selected from among:

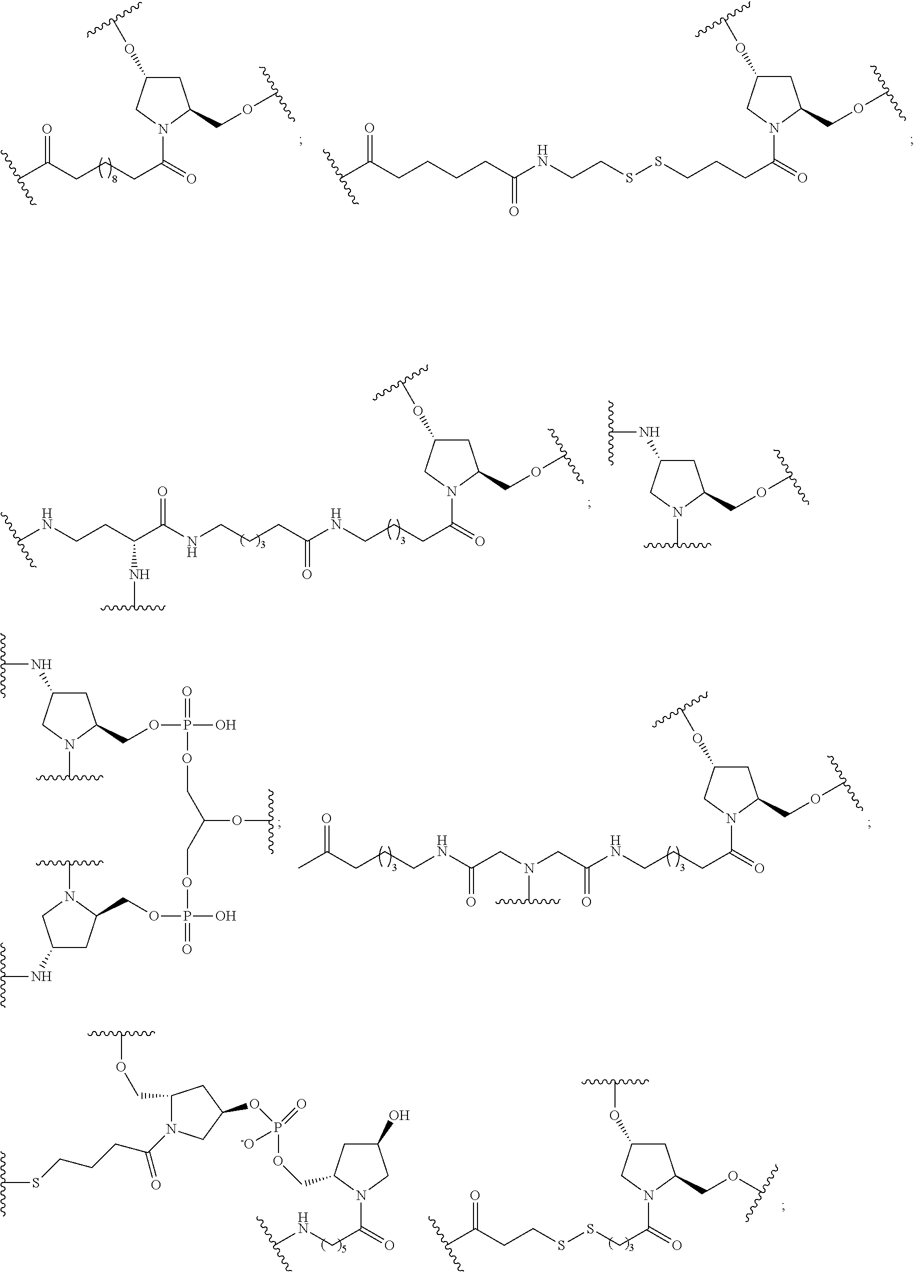

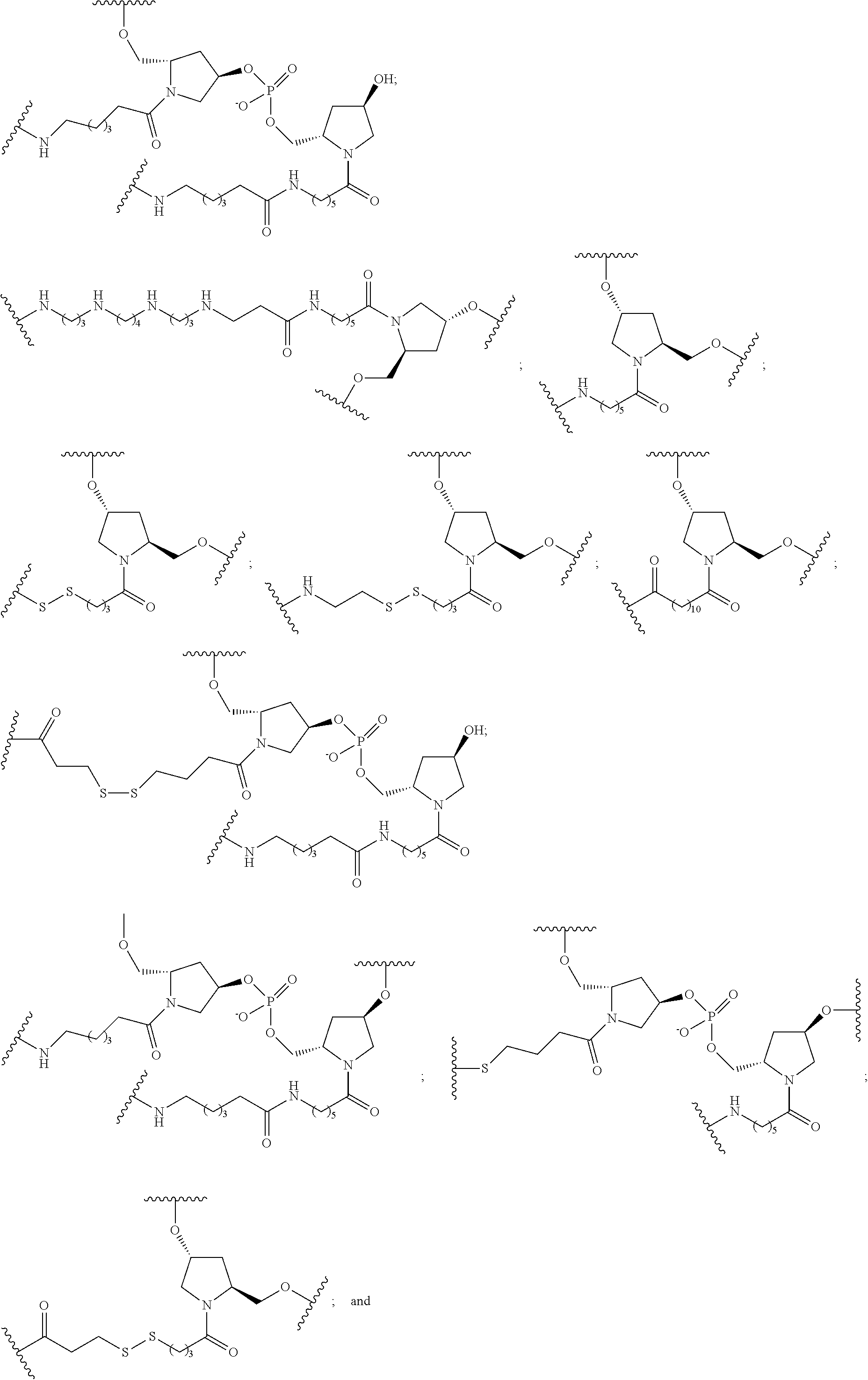

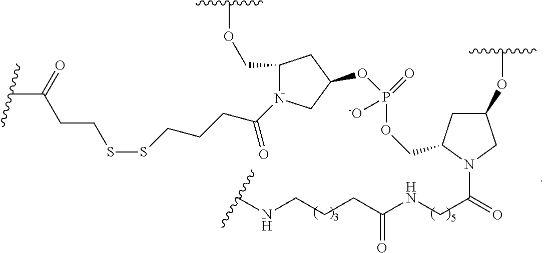

In certain embodiments, a linker has a structure selected from among:

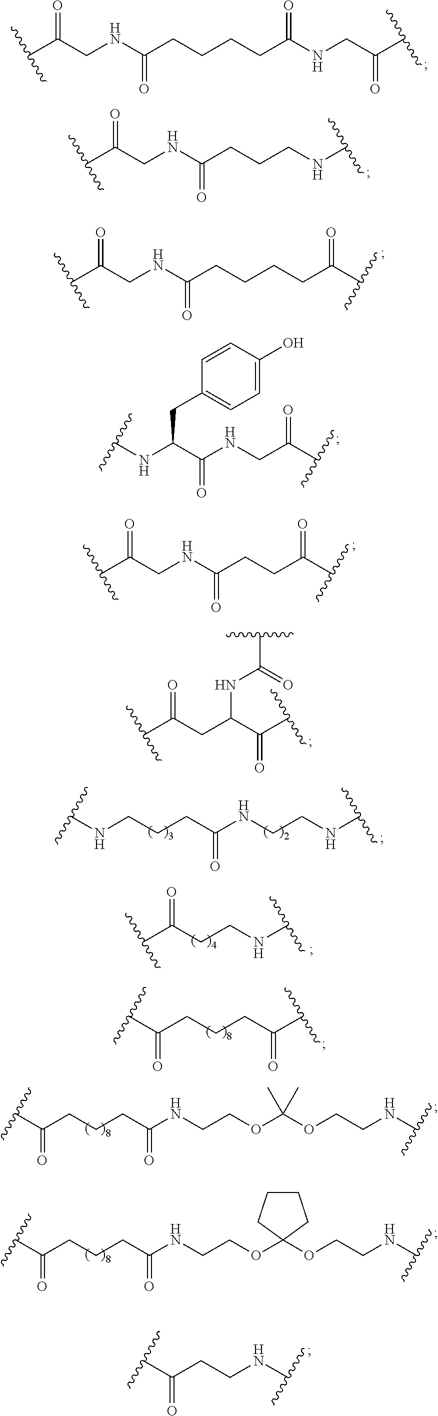

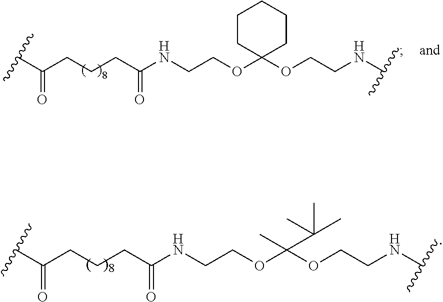

In certain embodiments, a linker has a structure selected from among:

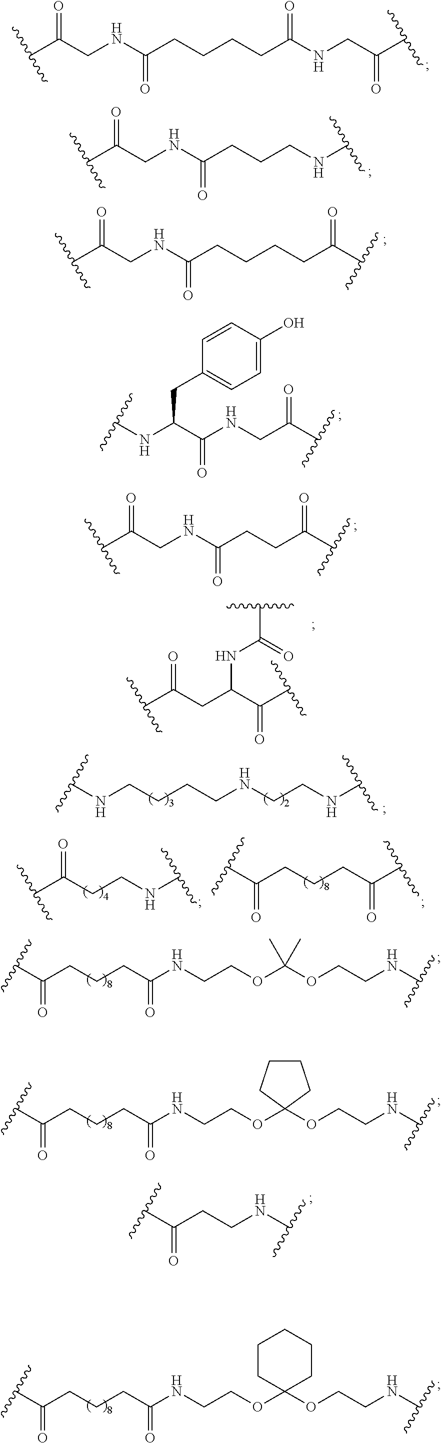

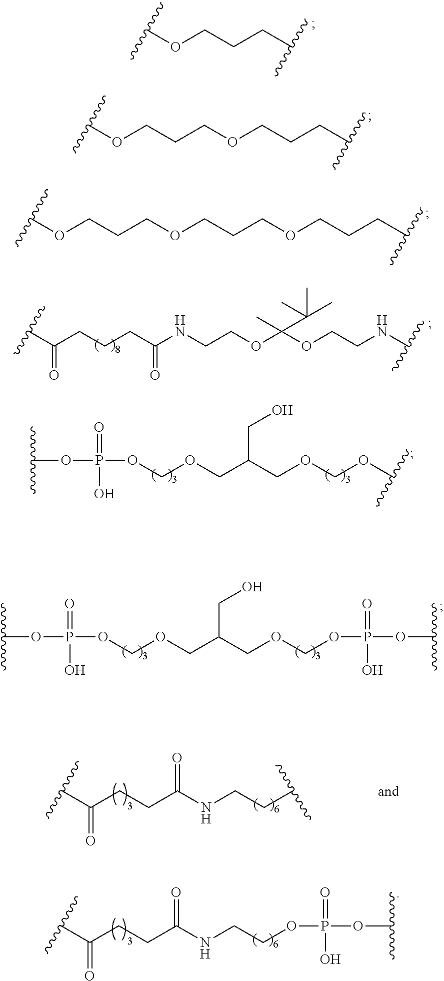

In certain embodiments, a linker has a structure selected from among:

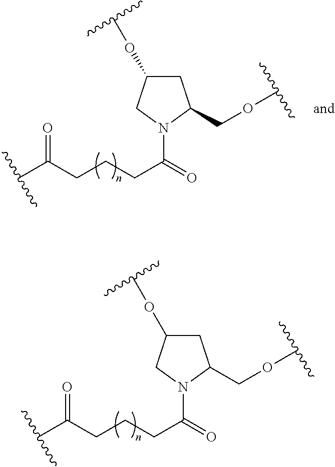

wherein n is from 1 to 20.

In certain embodiments, a linker has a structure selected from among:

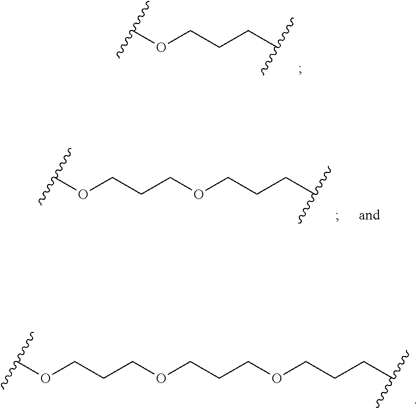 [SMILES: *CCCO* ; *CCCOCCCO* ; *CCCOCCCOCCCO*]

In certain embodiments, a linker has a structure selected from among:

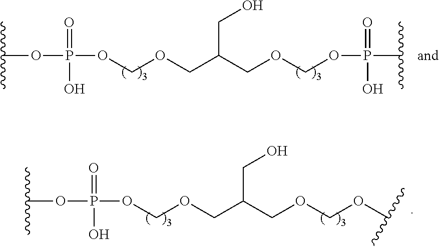

In certain embodiments, a linker has a structure selected from among:

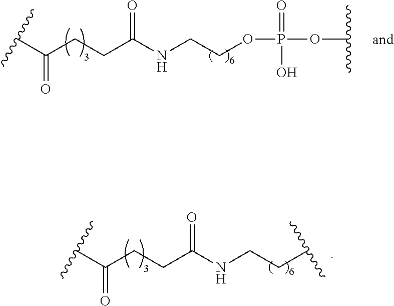

In certain embodiments, the conjugate linker has the structure:

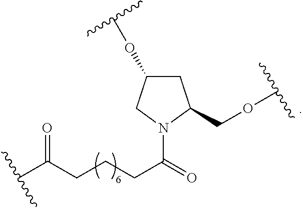

In certain embodiments, the conjugate linker has the structure:

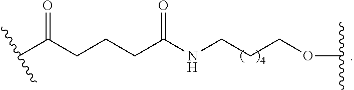

In certain embodiments, a linker has a structure selected from among:

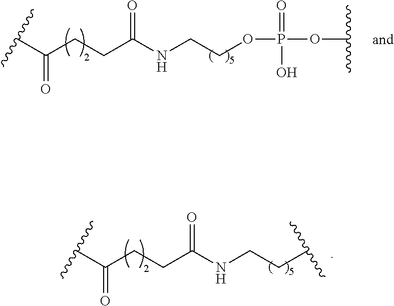

In certain embodiments, a linker has a structure selected from among:

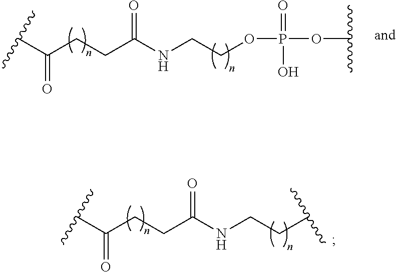

wherein each n is independently, 0, 1, 2, 3, 4, 5, 6, or 7.
   iii. Certain Cell-Targeting Moieties

In certain embodiments, conjugate groups comprise cell-targeting moieties. Certain such cell-targeting moieties increase cellular uptake of antisense compounds. In certain embodiments, cell-targeting moieties comprise a branching group, one or more tether, and one or more ligand. In certain embodiments, cell-targeting moieties comprise a branching group, one or more tether, one or more ligand and one or more cleavable bond.

### 1. Certain Branching Groups

In certain embodiments, the conjugate groups comprise a targeting moiety comprising a branching group and at least two tethered ligands. In certain embodiments, the branching group attaches the conjugate linker. In certain embodiments, the branching group attaches the cleavable moiety. In certain embodiments, the branching group attaches the antisense oligonucleotide. In certain embodiments, the branching group is covalently attached to the linker and each of the tethered ligands. In certain embodiments, the branching group comprises a branched aliphatic group comprising groups selected from alkyl, amide, disulfide, polyethylene glycol, ether, thioether and hydroxylamino groups. In certain embodiments, the branching group comprises groups selected from alkyl, amide and ether groups. In certain embodiments, the branching group comprises groups selected from alkyl and ether groups. In certain embodiments, the branching group comprises a mono or polycyclic ring system. In certain embodiments, the branching group comprises one or more cleavable bond. In certain embodiments, the conjugate group does not include a branching group.

In certain embodiments, a branching group has a structure selected from among:

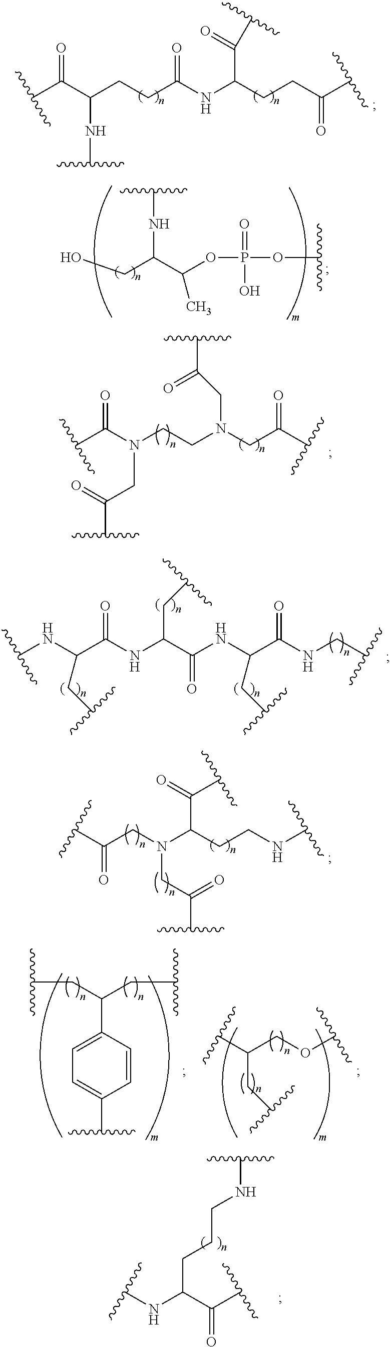

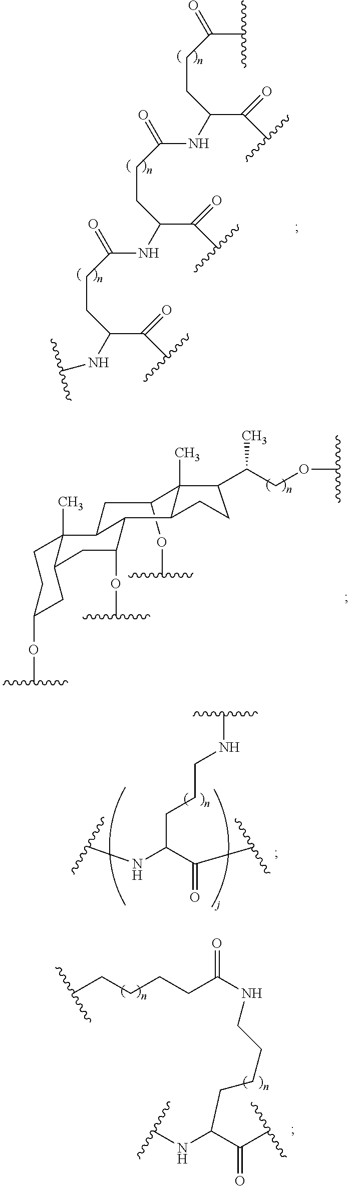

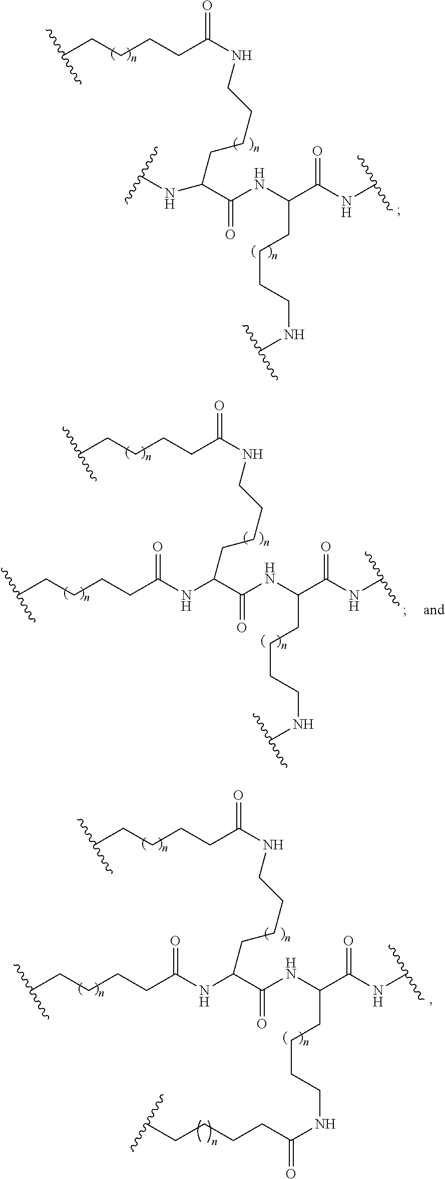

wherein each n is, independently, from 1 to 20;
j is from 1 to 3; and
m is from 2 to 6.

In certain embodiments, a branching group has a structure selected from among:

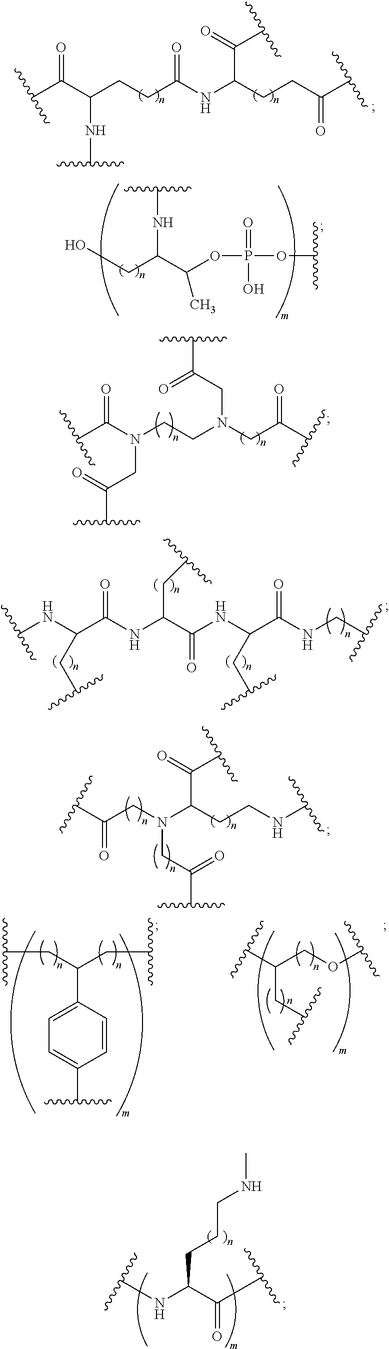

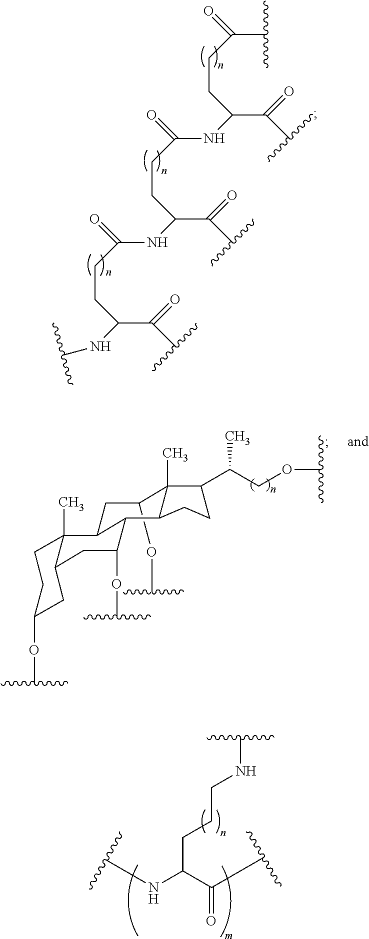

wherein each n is, independently, from 1 to 20; and
m is from 2 to 6.

In certain embodiments, a branching group has a structure selected from among:

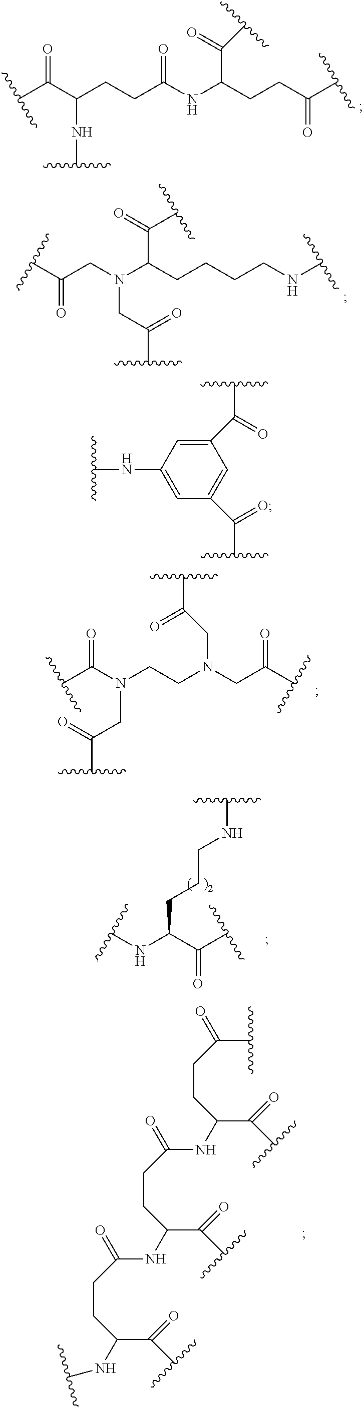

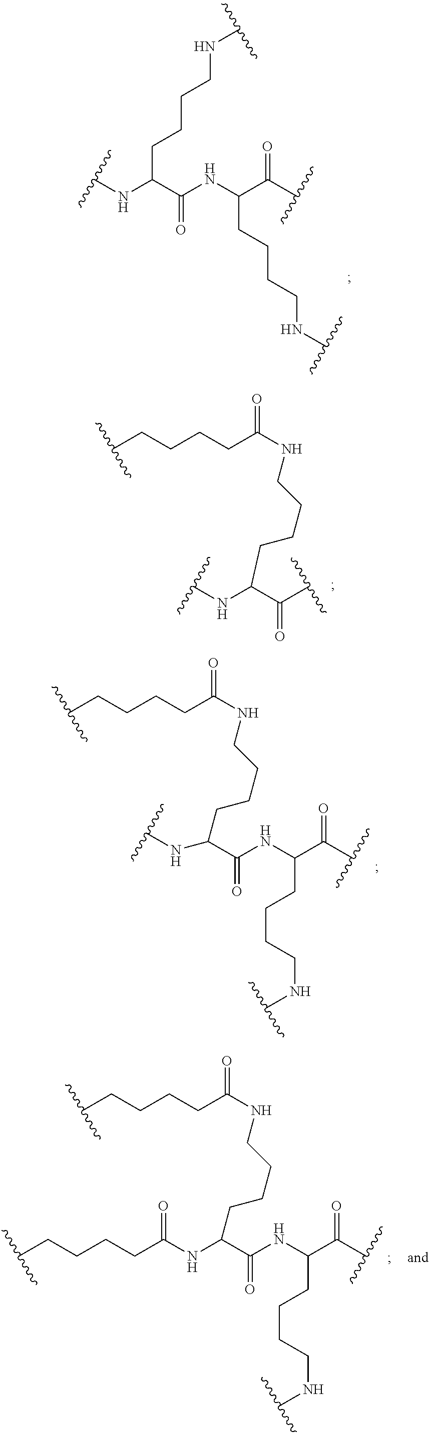 [SMILES: *NCCCCC(NC(=O)C(CCCCN*)N*)C(*)=O ; *CCCCC(=O)NCCCCC(N*)C(*)=O ; *CCCCC(=O)NCCCCC(N*)C(=O)NC(CCCCN*)C(*)=O ; *CCCCC(=O)NCCCCC(NC(=O)CCCC*)C(=O)NC(CCCCN*)C(*)=O]

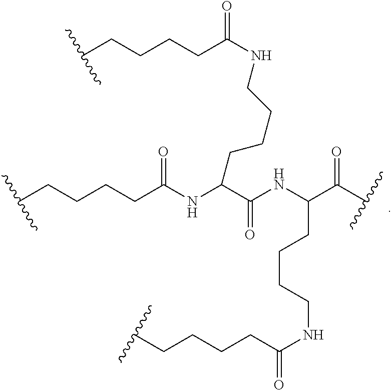 [SMILES: *CCCCC(=O)NCCCCC(NC(=O)C(CCCCNC(=O)CCCC*)NC(=O)CCCC*)C(*)=O]

In certain embodiments, a branching group has a structure selected from among:

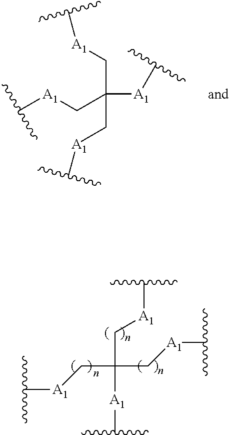

wherein each A₁ is independently, O, S, C═O or NH; and
each n is, independently, from 1 to 20.

In certain embodiments, a branching group has a structure selected from among:

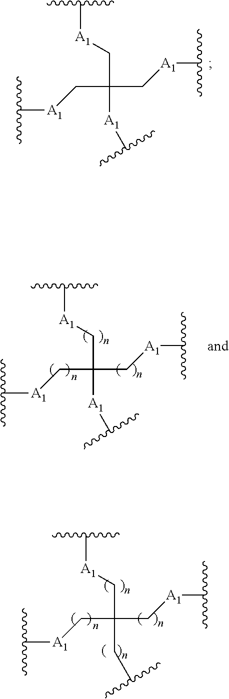

wherein each A₁ is independently, O, S, C═O or NH; and
each n is, independently, from 1 to 20.

In certain embodiments, a branching group has a structure selected from among:

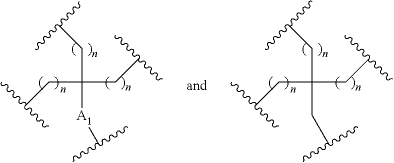

wherein A₁ is O, S, C═O or NH; and
each n is, independently, from 1 to 20.

In certain embodiments, a branching group has a structure selected from among:

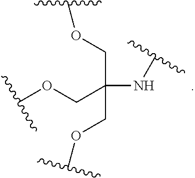 [SMILES: *NC(CO*)(CO*)CO*]

In certain embodiments, a branching group has a structure selected from among:

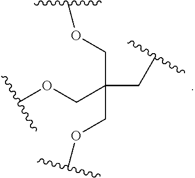 [SMILES: *CC(CO*)(CO*)CO*]

In certain embodiments, a branching group has a structure selected from among:

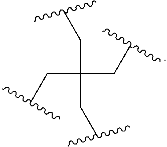 [SMILES: *CC(C*)(C*)C*]

### 2. Certain Tethers

In certain embodiments, conjugate groups comprise one or more tethers covalently attached to the branching group. In certain embodiments, conjugate groups comprise one or more tethers covalently attached to the linking group. In certain embodiments, each tether is a linear aliphatic group comprising one or more groups selected from alkyl, ether, thioether, disulfide, amide and polyethylene glycol groups in any combination. In certain embodiments, each tether is a linear aliphatic group comprising one or more groups selected from alkyl, substituted alkyl, ether, thioether, disulfide, amide, phosphodiester and polyethylene glycol groups in any combination. In certain embodiments, each tether is a linear aliphatic group comprising one or more groups selected from alkyl, ether and amide groups in any combination. In certain embodiments, each tether is a linear aliphatic group comprising one or more groups selected from alkyl, substituted alkyl, phosphodiester, ether and amide groups in any combination. In certain embodiments, each tether is a linear aliphatic group comprising one or more groups selected from alkyl and phosphodiester in any combination. In certain embodiments, each tether comprises at least one phosphorus linking group or neutral linking group.

In certain embodiments, the tether includes one or more cleavable bond. In certain embodiments, the tether is attached to the branching group through either an amide or an ether group. In certain embodiments, the tether is attached to the branching group through a phosphodiester group. In certain embodiments, the tether is attached to the branching group through a phosphorus linking group or neutral linking group. In certain embodiments, the tether is attached to the branching group through an ether group. In certain embodiments, the tether is attached to the ligand through either an amide or an ether group. In certain embodiments, the tether is attached to the ligand through an ether group. In certain embodiments, the tether is attached to the ligand through either an amide or an ether group. In certain embodiments, the tether is attached to the ligand through an ether group.

In certain embodiments, each tether comprises from about 8 to about 20 atoms in chain length between the ligand and the branching group. In certain embodiments, each tether group comprises from about 10 to about 18 atoms in chain length between the ligand and the branching group. In certain embodiments, each tether group comprises about 13 atoms in chain length.

In certain embodiments, a tether has a structure selected from among:

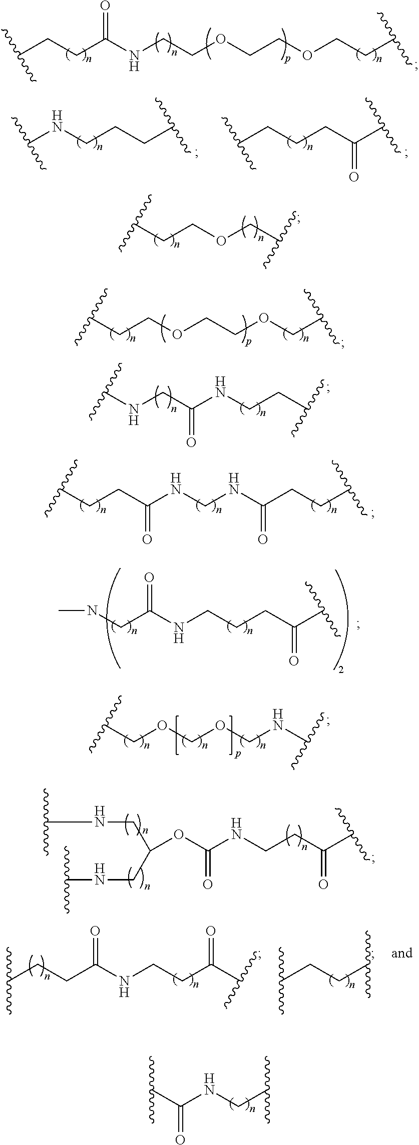

wherein each n is, independently, from 1 to 20; and
each p is from 1 to about 6.

In certain embodiments, a tether has a structure selected from among:

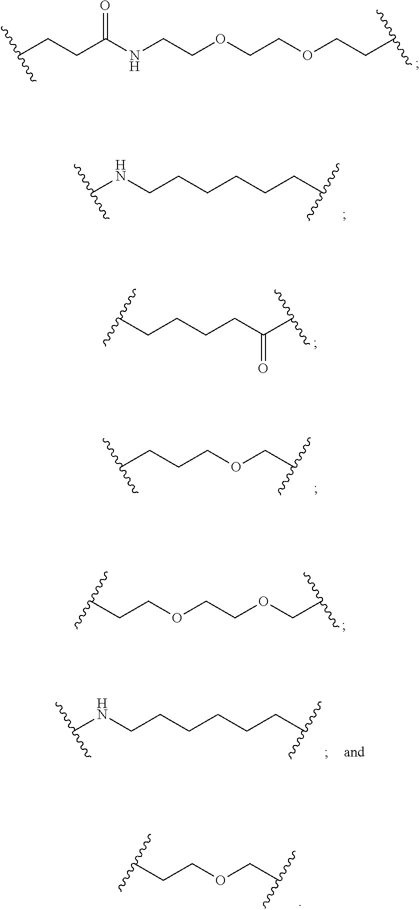

In certain embodiments, a tether has a structure selected from among:

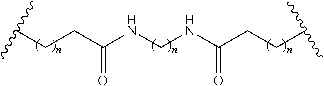

wherein each n is, independently, from 1 to 20.

In certain embodiments, a tether has a structure selected from among:

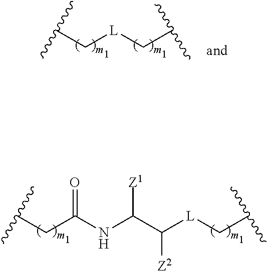

wherein L is either a phosphorus linking group or a neutral linking group;
Z₁ is C(═O)O—R₂;
Z₂ is H, C₁-C₆ alkyl or substituted C₁-C₆ alky;
R₂ is H, C₁-C₆ alkyl or substituted C₁-C₆ alky; and
each m₁ is, independently, from 0 to 20 wherein at least one m₁ is greater than 0 for each tether.

In certain embodiments, a tether has a structure selected from among:

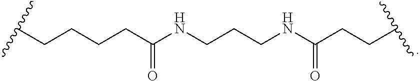 [SMILES: *CCCCC(=O)NCCCNC(=O)CC*]

In certain embodiments, a tether has a structure selected from among:

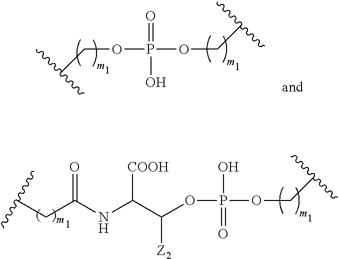

wherein Z₂ is H or CH₃; and
each m₁ is, independently, from 0 to 20 wherein at least one m₁ is greater than 0 for each tether.

In certain embodiments, a tether has a structure selected from among:

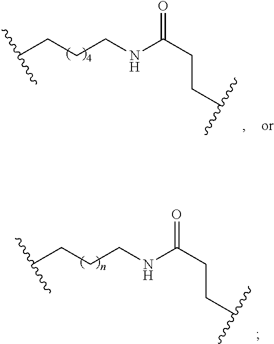

wherein each n is independently, 0, 1, 2, 3, 4, 5, 6, or 7.

In certain embodiments, a tether comprises a phosphorus linking group. In certain embodiments, a tether does not comprise any amide bonds. In certain embodiments, a tether comprises a phosphorus linking group and does not comprise any amide bonds.

### 3. Certain Ligands

In certain embodiments, the present disclosure provides ligands wherein each ligand is covalently attached to a tether. In certain embodiments, each ligand is selected to have an affinity for at least one type of receptor on a target cell. In certain embodiments, ligands are selected that have an affinity for at least one type of receptor on the surface of a mammalian liver cell. In certain embodiments, ligands are selected that have an affinity for the hepatic asialoglycoprotein receptor (ASGP-R). In certain embodiments, each ligand is a carbohydrate. In certain embodiments, each ligand is, independently selected from galactose, N-acetyl galactoseamine, mannose, glucose, glucosamone and fucose. In certain embodiments, each ligand is N-acetyl galactoseamine (GalNAc). In certain embodiments, the targeting moiety comprises 2 to 6 ligands. In certain embodiments, the targeting moiety comprises 3 ligands. In certain embodiments, the targeting moiety comprises 3 N-acetyl galactoseamine ligands.

In certain embodiments, the ligand is a carbohydrate, carbohydrate derivative, modified carbohydrate, multivalent carbohydrate cluster, polysaccharide, modified polysaccharide, or polysaccharide derivative. In certain embodiments, the ligand is an amino sugar or a thio sugar. For example, amino sugars may be selected from any number of compounds known in the art, for example glucosamine, sialic acid, α-D-galactosamine, N-Acetylgalactosamine, 2-acetamido-2-deoxy-D-galactopyranose (GalNAc), 2-Amino-3-O—[(R)-1-carboxyethyl]-2-deoxy-β-D-glucopyranose (0-muramic acid), 2-Deoxy-2-methylamino-L-glucopyranose, 4,6-Dideoxy-4-formamido-2,3-di-O-methyl-D-mannopyranose, 2-Deoxy-2-sulfoamino-D-glucopyranose and N-sulfo-D-glucosamine, and N-Glycoloyl-α-neuraminic acid. For example, thio sugars may be selected from the group consisting of 5-Thio-β-D-glucopyranose, Methyl 2,3,4-tri-O-acetyl-1-thio-6-O-trityl-α-D-glucopyranoside, 4-Thio-β-D-galactopyranose, and ethyl 3,4,6,7-tetra-O-acetyl-2-deoxy-1,5-dithio-α-D-gluco-heptopyranoside.

In certain embodiments, “GalNac” or “Gal-NAc” refers to 2-(Acetylamino)-2-deoxy-D-galactopyranose, commonly referred to in the literature as N-acetyl galactosamine. In certain embodiments, “N-acetyl galactosamine” refers to 2-(Acetylamino)-2-deoxy-D-galactopyranose. In certain embodiments, “GalNac” or “Gal-NAc” refers to 2-(Acetylamino)-2-deoxy-D-galactopyranose. In certain embodiments, “GalNac” or “Gal-NAc” refers to 2-(Acetylamino)-2-deoxy-D-galactopyranose, which includes both the β-form: 2-(Acetylamino)-2-deoxy-β-D-galactopyranose and α-form: 2-(Acetylamino)-2-deoxy-D-galactopyranose. In certain embodiments, both the β-form: 2-(Acetylamino)-2-deoxy-β-D-galactopyranose and α-form: 2-(Acetylamino)-2-deoxy-D-galactopyranose may be used interchangeably. Accordingly, in structures in which one form is depicted, these structures are intended to include the other form as well. For example, where the structure for an α-form: 2-(Acetylamino)-2-deoxy-D-galactopyranose is shown, this structure is intended to include the other form as well. In certain embodiments, In certain preferred embodiments, the β-form 2-(Acetylamino)-2-deoxy-D-galactopyranose is the preferred embodiment.

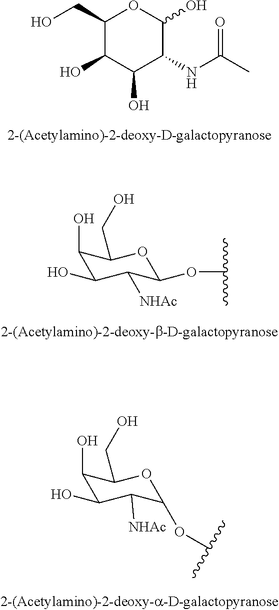 [SMILES: CC(=O)N[C@H]1C(O)O[C@H](CO)[C@H](O)[C@@H]1O ; *OC1OC(CO)C(O)C(O)C1NC(C)=O ; *OC1OC(CO)C(O)C(O)C1NC(C)=O]

In certain embodiments one or more ligand has a structure selected from among:

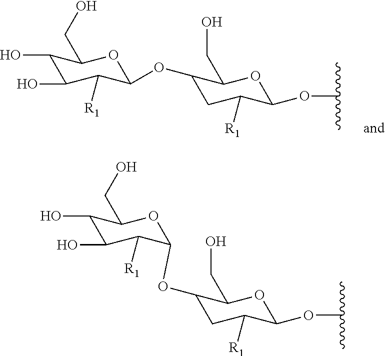

wherein each R₁ is selected from OH and NHCOOH.

In certain embodiments one or more ligand has a structure selected from among:

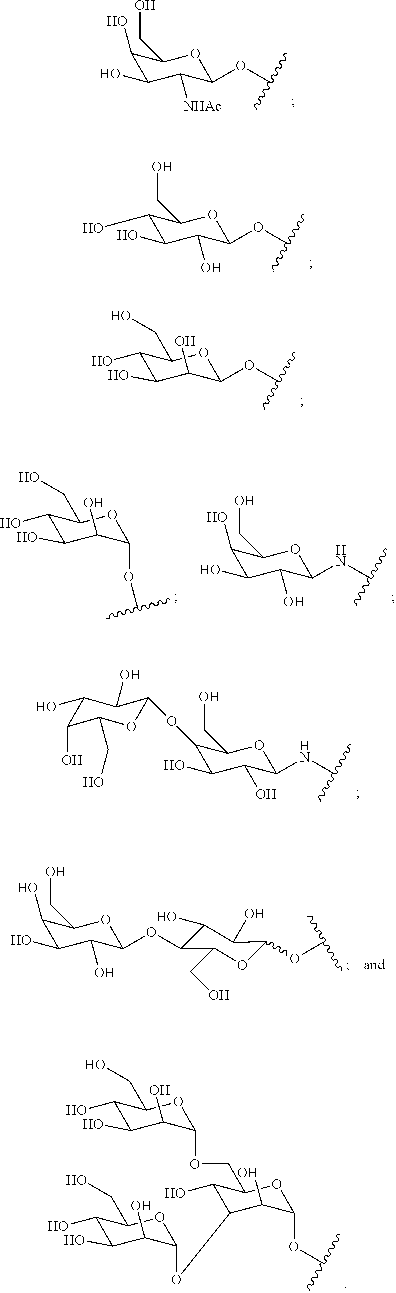

In certain embodiments one or more ligand has a structure selected from among:

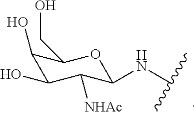 [SMILES: *NC1OC(CO)C(O)C(O)C1NC(C)=O]

In certain embodiments one or more ligand has a structure selected from among:

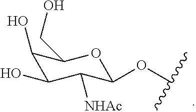 [SMILES: *OC1OC(CO)C(O)C(O)C1NC(C)=O]

i. Certain Conjugates

In certain embodiments, conjugate groups comprise the structural features above. In certain such embodiments, conjugate groups have the following structure:

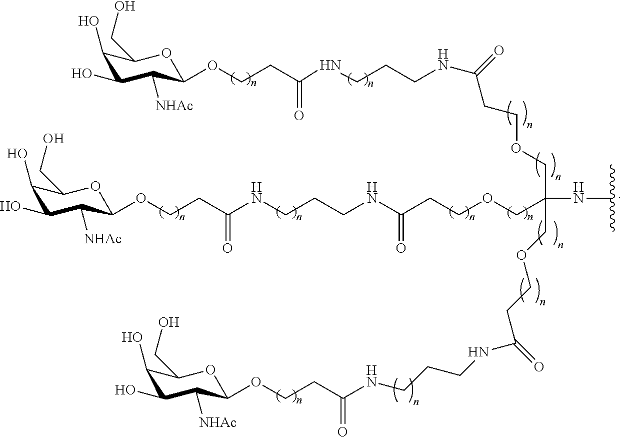

wherein each n is, independently, from 1 to 20.

In certain such embodiments, conjugate groups have the following structure:

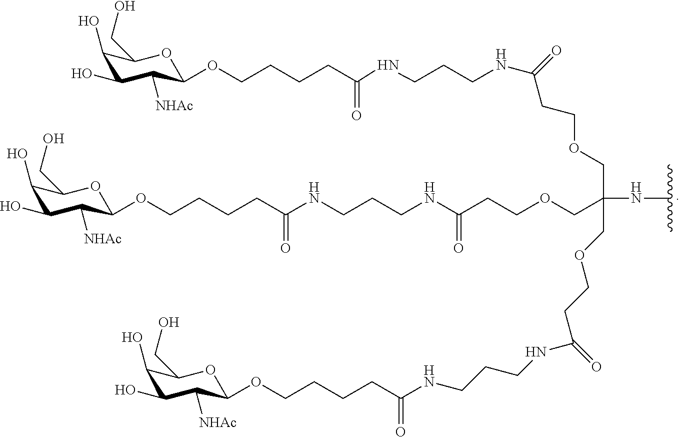

In certain such embodiments, conjugate groups have the following structure:

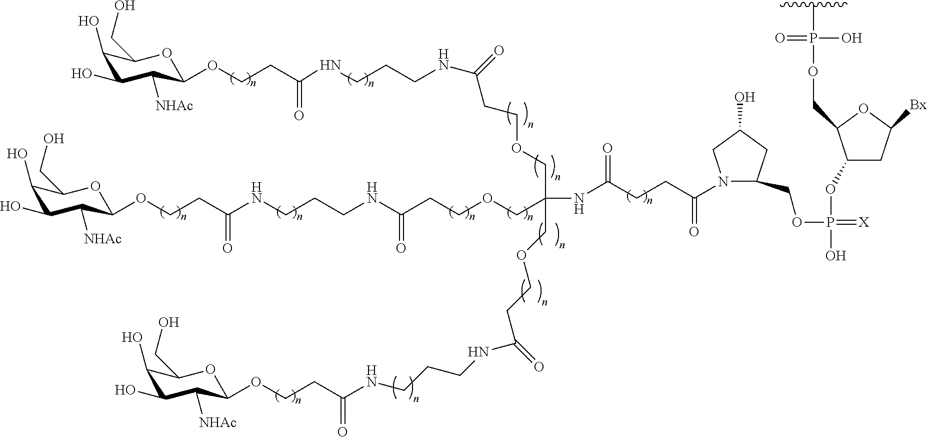

wherein each n is, independently, from 1 to 20;
Z is H or a linked solid support;
Q is an antisense compound;
X is O or S; and
Bx is a heterocyclic base moiety.

In certain such embodiments, conjugate groups have the following structure:

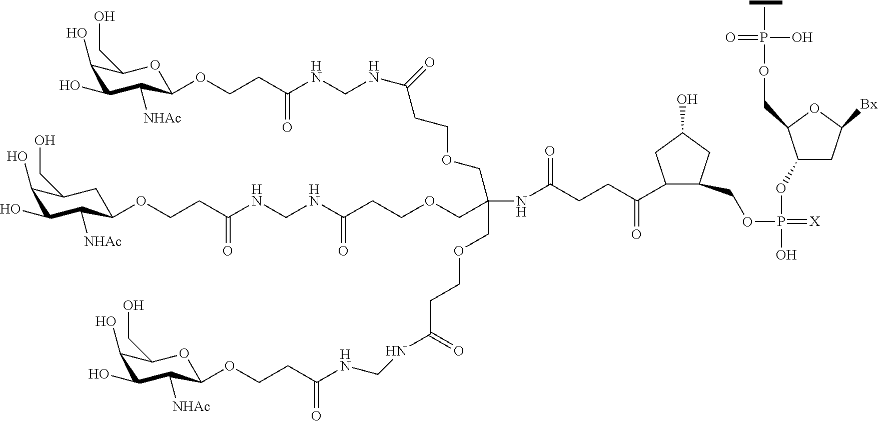

In certain such embodiments, conjugate groups have the following structure:

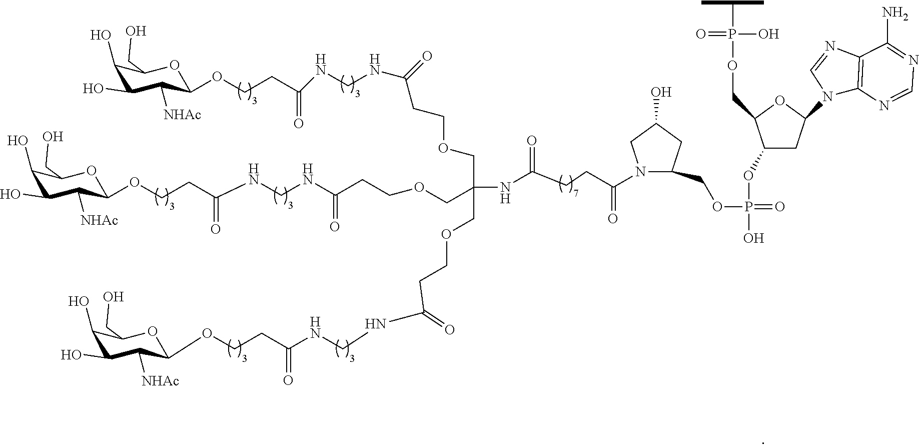

In certain such embodiments, conjugate groups have the following structure:

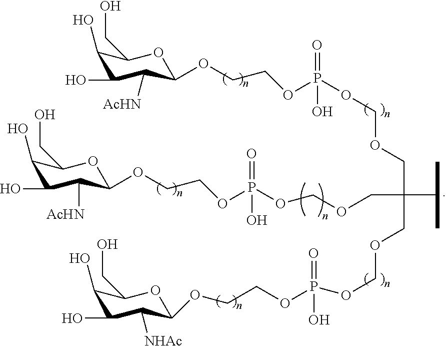

In certain such embodiments, conjugate groups have the following structure:

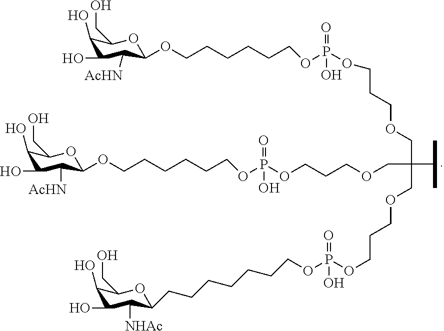

In certain such embodiments, conjugate groups have the following structure:

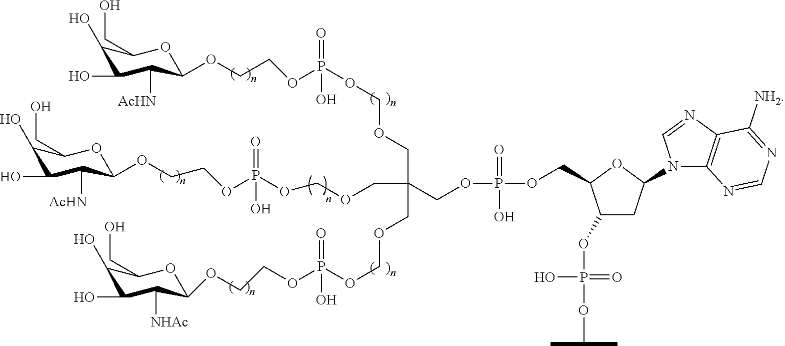

In certain such embodiments, conjugate groups have the following structure:

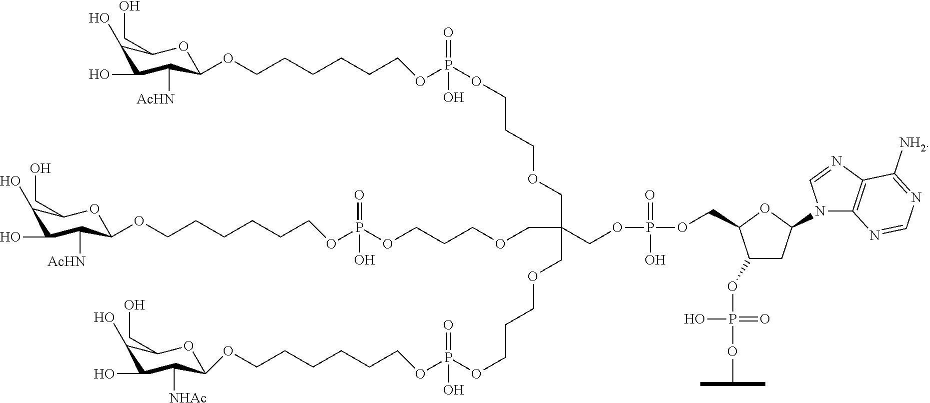

In certain such embodiments, conjugate groups have the following structure:

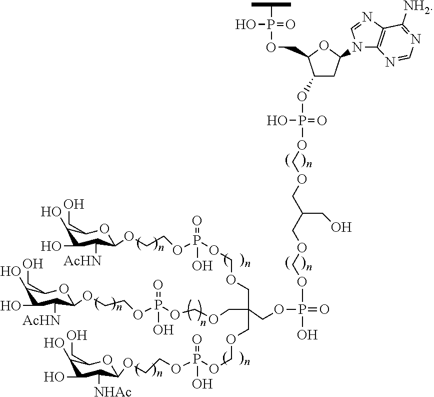

In certain such embodiments, conjugate groups have the following structure:

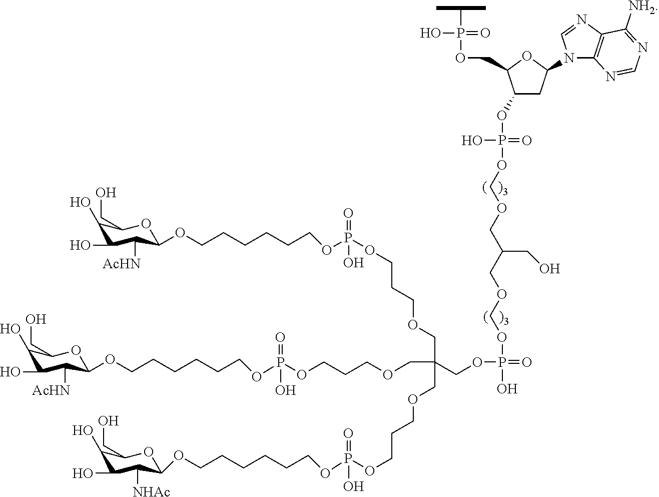

In certain embodiments, conjugates do not comprise a pyrrolidine.

In certain such embodiments, conjugate groups have the following structure:

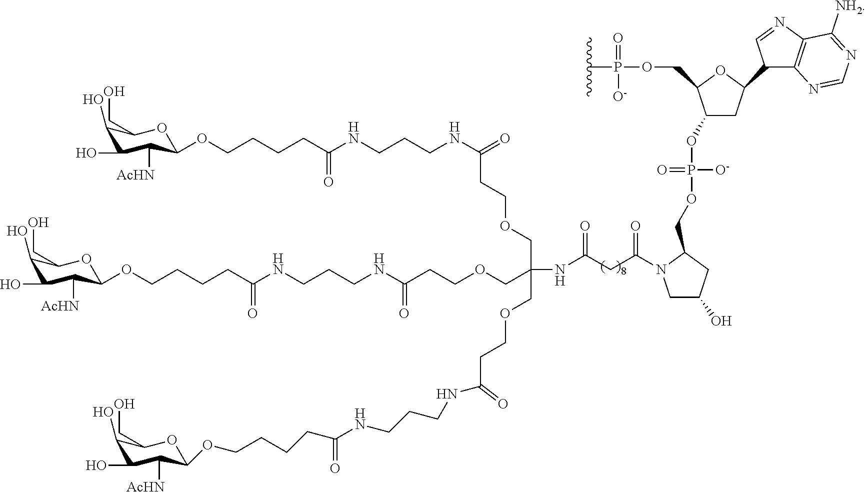

In certain such embodiments, conjugate groups have the following structure:

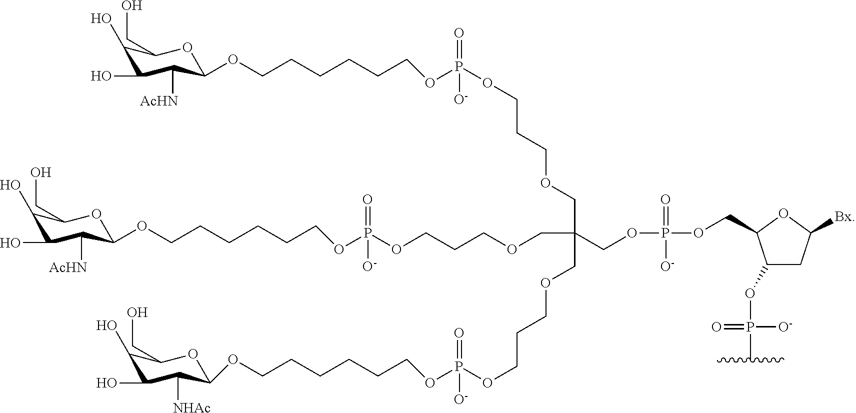

In certain such embodiments, conjugate groups have the following structure:

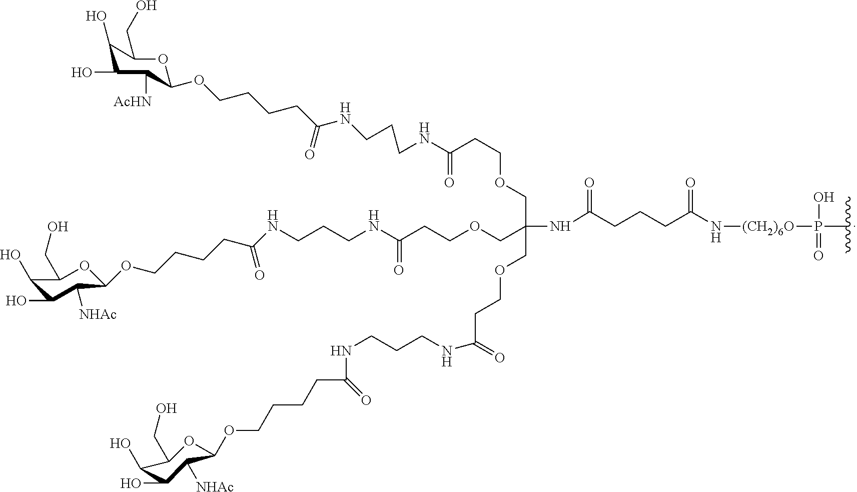

In certain such embodiments, conjugate groups have the following structure:

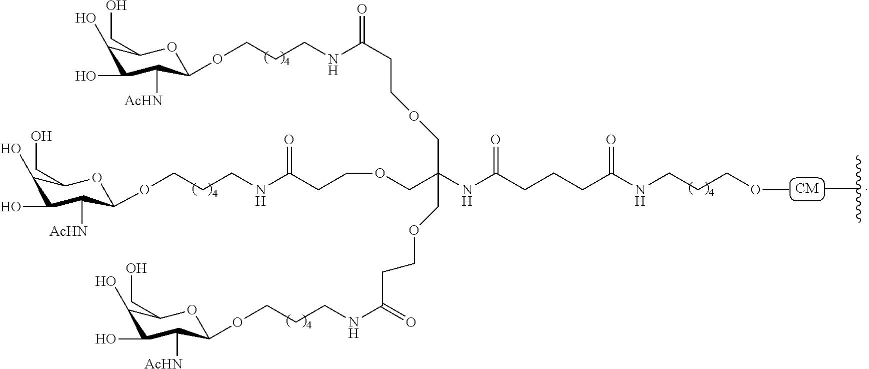

In certain such embodiments, conjugate groups have the following structure:

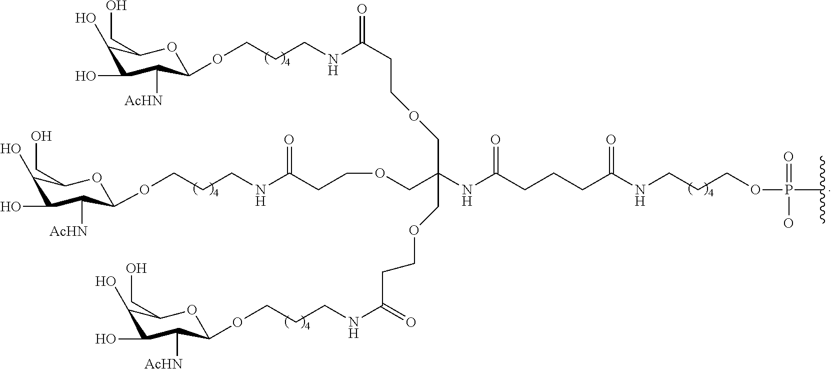

In certain such embodiments, conjugate groups have the following structure:

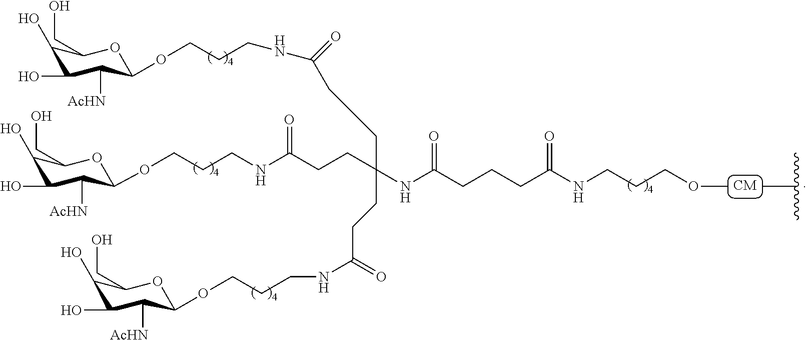

In certain such embodiments, conjugate groups have the following structure:

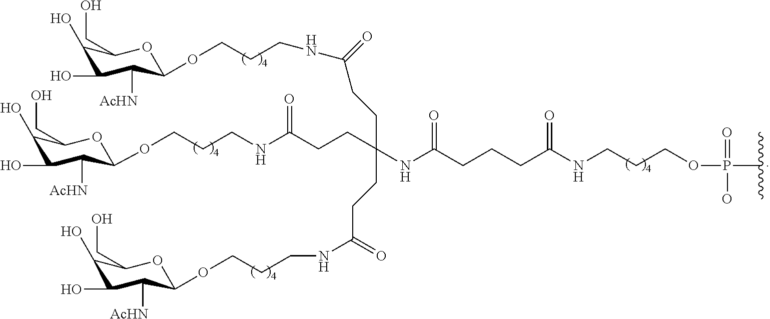

In certain such embodiments, conjugate groups have the following structure:

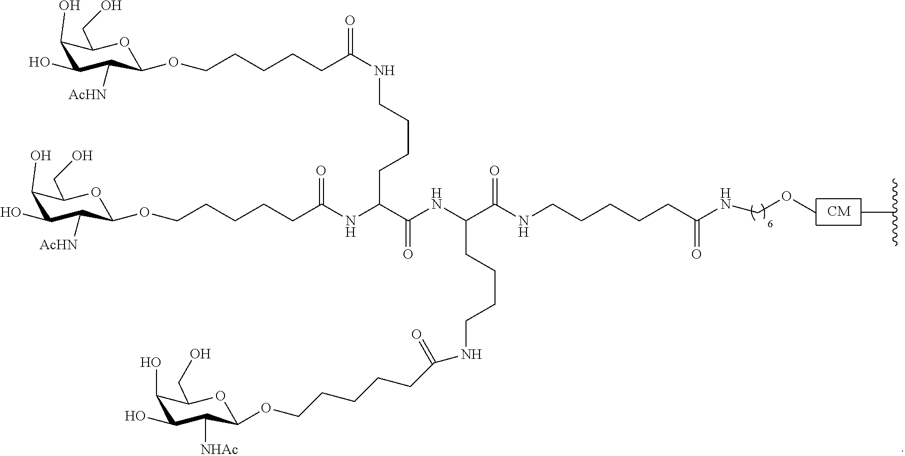

In certain such embodiments, conjugate groups have the following structure:

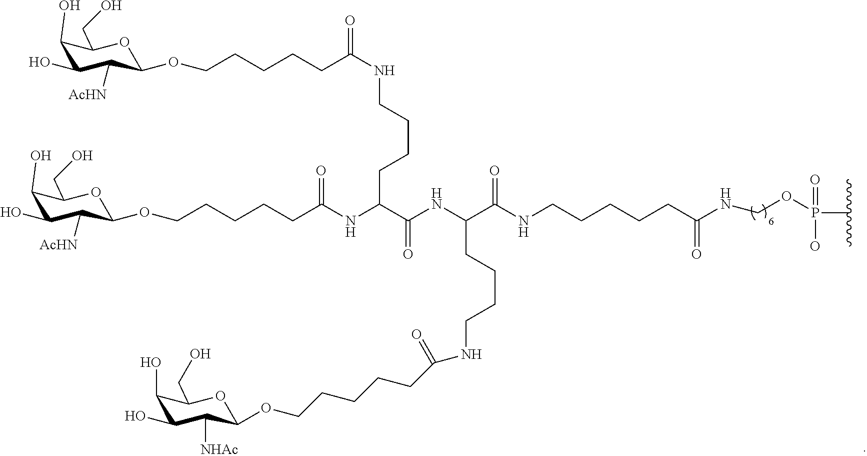

In certain such embodiments, conjugate groups have the following structure:

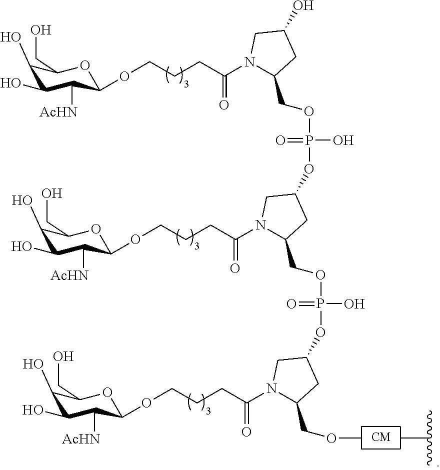

In certain such embodiments, conjugate groups have the following structure:

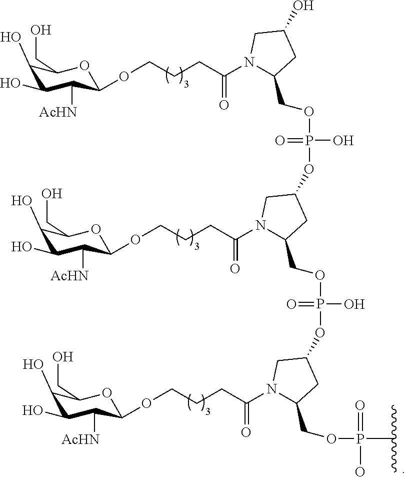

In certain embodiments, the cell-targeting moiety of the conjugate group has the following structure:

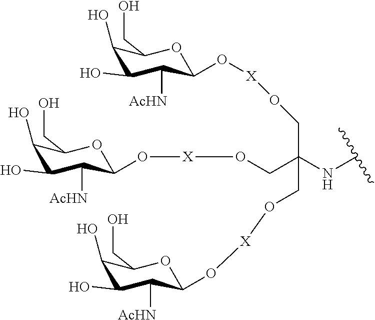

wherein X is a substituted or unsubstituted tether of six to eleven consecutively bonded atoms.

In certain embodiments, the cell-targeting moiety of the conjugate group has the following structure:

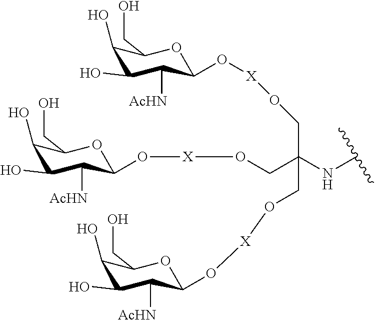

wherein X is a substituted or unsubstituted tether of ten consecutively bonded atoms.

In certain embodiments, the cell-targeting moiety of the conjugate group has the following structure:

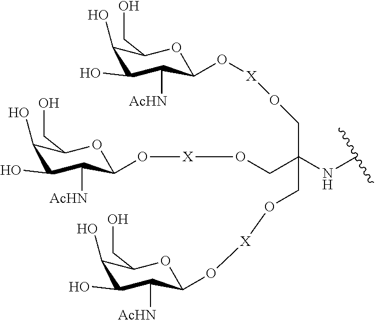

wherein X is a substituted or unsubstituted tether of four to eleven consecutively bonded atoms and
wherein the tether comprises exactly one amide bond.

In certain embodiments, the cell-targeting moiety of the conjugate group has the following structure:

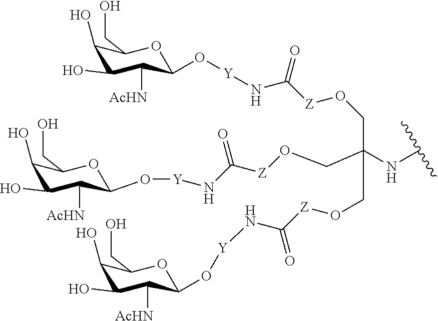

wherein Y and Z are independently selected from a C₁-C₁₂ substituted or unsubstituted alkyl, alkenyl, or alkynyl group, or a group comprising an ether, a ketone, an amide, an ester, a carbamate, an amine, a piperidine, a phosphate, a phosphodiester, a phosphorothioate, a triazole, a pyrrolidine, a disulfide, or a thioether.

In certain such embodiments, the cell-targeting moiety of the conjugate group has the following structure:

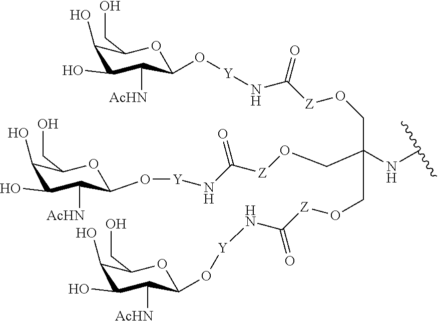

wherein Y and Z are independently selected from a C₁-C₁₂ substituted or unsubstituted alkyl group, or a group comprising exactly one ether or exactly two ethers, an amide, an amine, a piperidine, a phosphate, a phosphodiester, or a phosphorothioate.

In certain such embodiments, the cell-targeting moiety of the conjugate group has the following structure:

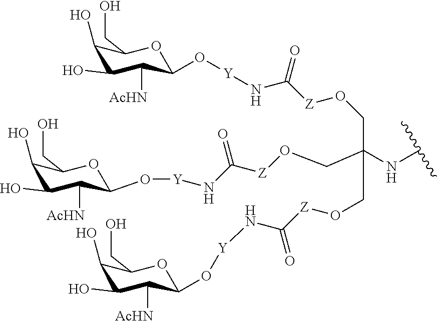

wherein Y and Z are independently selected from a C₁-C₁₂ substituted or unsubstituted alkyl group.

In certain such embodiments, the cell-targeting moiety of the conjugate group has the following structure:

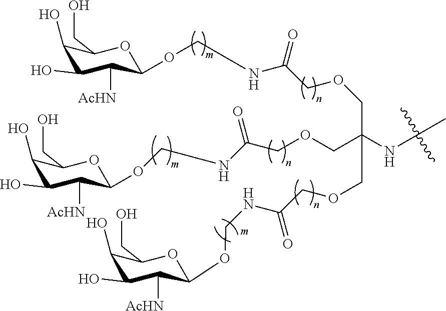

wherein m and n are independently selected from 1, 2, 3, 4, 5, 6, 7, 8, 9, 10, 11, and 12.

In certain such embodiments, the cell-targeting moiety of the conjugate group has the following structure:

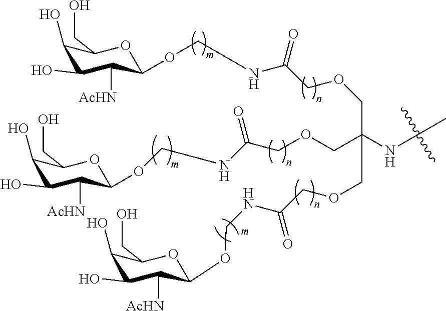

wherein m is 4, 5, 6, 7, or 8, and n is 1, 2, 3, or 4.

In certain embodiments, the cell-targeting moiety of the conjugate group has the following structure:

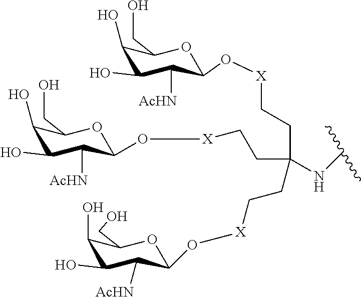

wherein X is a substituted or unsubstituted tether of four to thirteen consecutively bonded atoms, and wherein X does not comprise an ether group.

In certain embodiments, the cell-targeting moiety of the conjugate group has the following structure:

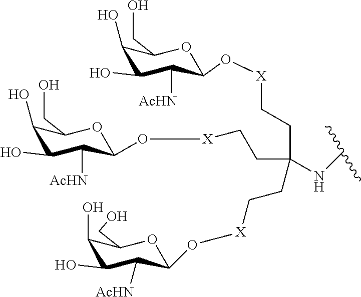

wherein X is a substituted or unsubstituted tether of eight consecutively bonded atoms, and wherein X does not comprise an ether group.

In certain embodiments, the cell-targeting moiety of the conjugate group has the following structure:

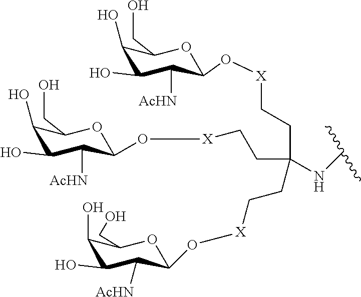

wherein X is a substituted or unsubstituted tether of four to thirteen consecutively bonded atoms, and wherein the tether comprises exactly one amide bond, and wherein X does not comprise an ether group.

In certain embodiments, the cell-targeting moiety of the conjugate group has the following structure:

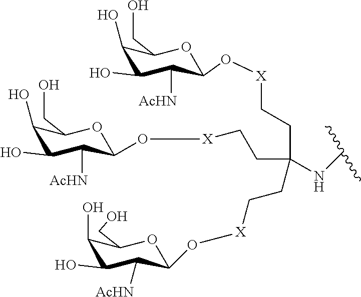

wherein X is a substituted or unsubstituted tether of four to thirteen consecutively bonded atoms and wherein the tether consists of an amide bond and a substituted or unsubstituted C₂-C₁₁ alkyl group.

In certain embodiments, the cell-targeting moiety of the conjugate group has the following structure:

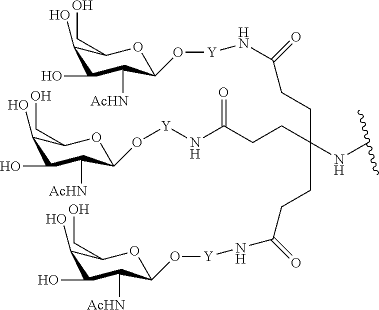

wherein is selected from a C₁-C₁₂ substituted or unsubstituted alkyl, alkenyl, or alkynyl group, or a group comprising an ether, a ketone, an amide, an ester, a carbamate, an amine, a piperidine, a phosphate, a phosphodiester, a phosphorothioate, a triazole, a pyrrolidine, a disulfide, or a thioether.

In certain such embodiments, the cell-targeting moiety of the conjugate group has the following structure:

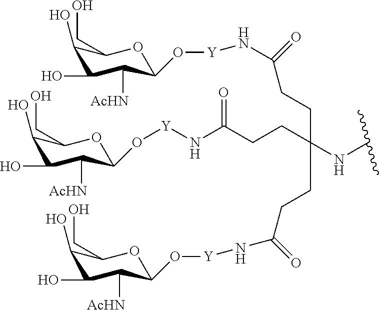

wherein Y is selected rom a C₁-C₁₂ substituted or unsubstituted alkyl group, or a group comprising an ether, an amine, a piperidine, a phosphate, a phosphodiester, or a phosphorothioate.

In certain such embodiments, the cell-targeting moiety of the conjugate group has the following structure:

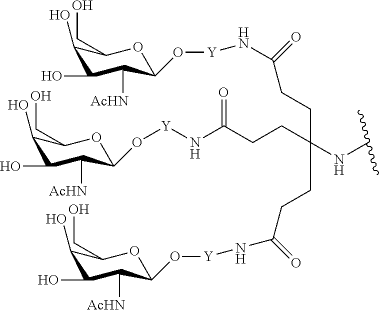

wherein Y is selected from a C1-C12 substituted or unsubstituted alkyl group.

In certain such embodiments, the cell-targeting moiety of the conjugate group has the following structure:

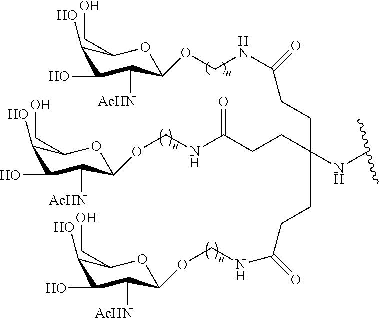

### Wherein n is 1, 2, 3, 4, 5, 6, 7, 8, 9, 10, 11, or 12.

In certain such embodiments, the cell-targeting moiety of the conjugate group has the following structure:

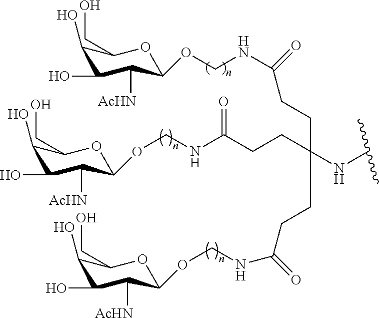

wherein n is 4, 5, 6, 7, or 8.

In certain embodiments, conjugates do not comprise a pyrrolidine.

### a Certain Conjugated Antisense Compounds

In certain embodiments, the conjugates are bound to a nucleoside of the antisense oligonucleotide at the 2′, 3′, of 5′ position of the nucleoside. In certain embodiments, a conjugated antisense compound has the following structure:

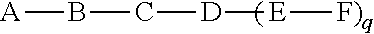

wherein
A is the antisense oligonucleotide;
B is the cleavable moiety
C is the conjugate linker
D is the branching group
each E is a tether;
each F is a ligand; and
q is an integer between 1 and 5.

In certain embodiments, a conjugated antisense compound has the following structure:

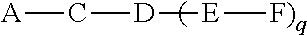

wherein
A is the antisense oligonucleotide;
C is the conjugate linker
D is the branching group
each E is a tether;
each F is a ligand; and
q is an integer between 1 and 5.

In certain such embodiments, the conjugate linker comprises at least one cleavable bond.

In certain such embodiments, the branching group comprises at least one cleavable bond.

In certain embodiments each tether comprises at least one cleavable bond.

In certain embodiments, the conjugates are bound to a nucleoside of the antisense oligonucleotide at the 2′, 3′, of 5′ position of the nucleoside.

In certain embodiments, a conjugated antisense compound has the following structure:

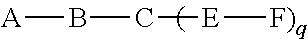

wherein
A is the antisense oligonucleotide;
B is the cleavable moiety
C is the conjugate linker
each E is a tether;
each F is a ligand; and
q is an integer between 1 and 5.

In certain embodiments, the conjugates are bound to a nucleoside of the antisense oligonucleotide at the 2′, 3′, of 5′ position of the nucleoside. In certain embodiments, a conjugated antisense compound has the following structure:

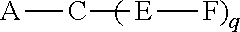

wherein
A is the antisense oligonucleotide;
C is the conjugate linker
each E is a tether;
each F is a ligand; and
q is an integer between 1 and 5.

In certain embodiments, a conjugated antisense compound has the following structure:

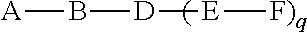

wherein
A is the antisense oligonucleotide;
B is the cleavable moiety
D is the branching group
each E is a tether;
each F is a ligand; and
q is an integer between 1 and 5.

In certain embodiments, a conjugated antisense compound has the following structure:

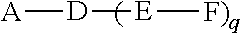

wherein
A is the antisense oligonucleotide;
D is the branching group
each E is a tether;
each F is a ligand; and
q is an integer between 1 and 5.

In certain such embodiments, the conjugate linker comprises at least one cleavable bond.

In certain embodiments each tether comprises at least one cleavable bond.

In certain embodiments, a conjugated antisense compound has a structure selected from among the following:

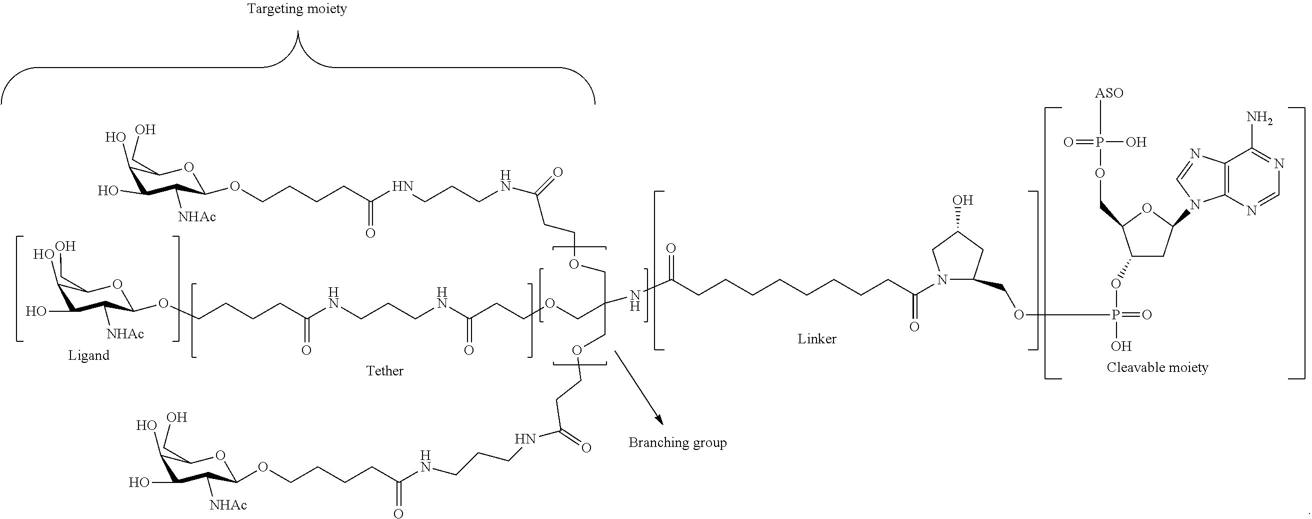

In certain embodiments, a conjugated antisense compound has a structure selected from among the following:

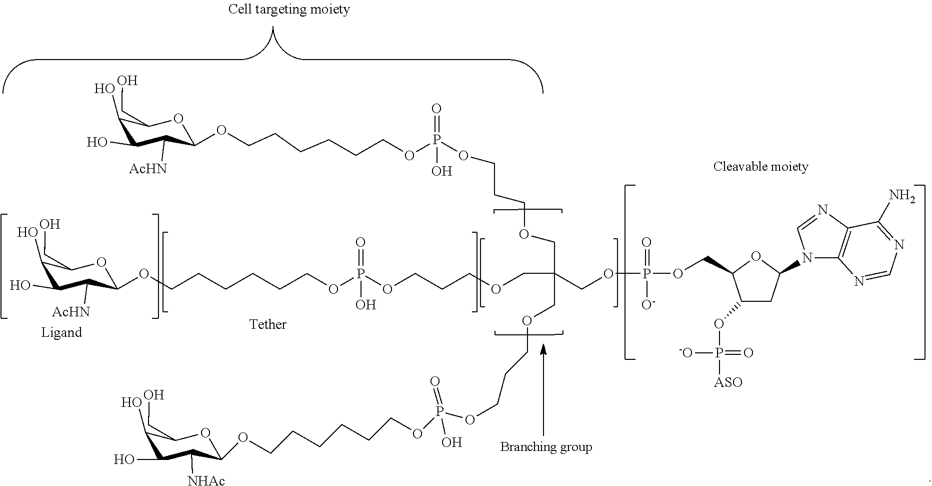

In certain embodiments, a conjugated antisense compound has a structure selected from among the following:

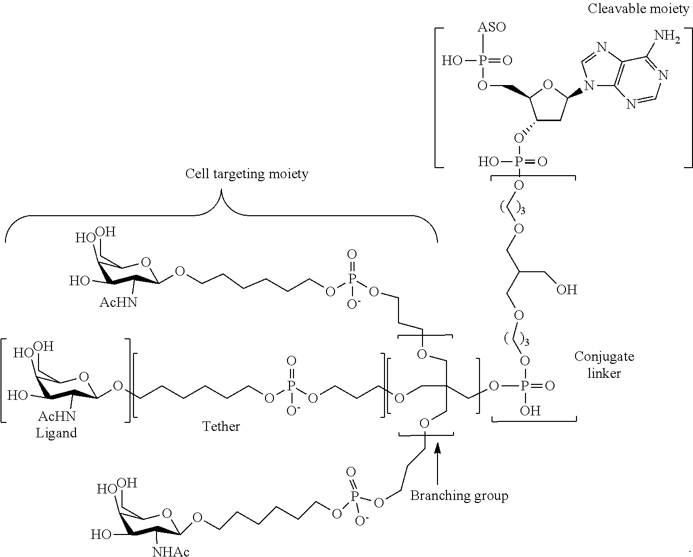

Representative United States patents, United States patent application publications, and international patent application publications that teach the preparation of certain of the above noted conjugates, conjugated antisense compounds, tethers, linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, each of which is incorporated by reference herein in its entirety.

Representative publications that teach the preparation of certain of the above noted conjugates, conjugated antisense compounds, tethers, linkers, branching groups, ligands, cleavable moieties as well as other modifications include without limitation, BIESSEN et al., “The Cholesterol Derivative of a Triantennary Galactoside with High Affinity for the Hepatic Asialoglycoprotein Receptor: a Potent Cholesterol Lowering Agent” J. Med. Chem. (1995) 38:1846-1852, BIESSEN et al., “Synthesis of Cluster Galactosides with High Affinity for the Hepatic Asialoglycoprotein Receptor” J. Med. Chem. (1995) 38:1538-1546, LEE et al., “New and more efficient multivalent glyco-ligands for asialoglycoprotein receptor of mammalian hepatocytes” Bioorganic & Medicinal Chemistry (2011) 19:2494-2500, RENSEN et al., “Determination of the Upper Size Limit for Uptake and Processing of Ligands by the Asialoglycoprotein Receptor on Hepatocytes in Vitro and in Vivo” J. Biol. Chem. (2001) 276(40):37577-37584, RENSEN et al., “Design and Synthesis of Novel N-Acetylgalactosamine-Terminated Glycolipids for Targeting of Lipoproteins to the Hepatic Asialoglycoprotein Receptor” J. Med. Chem. (2004) 47:5798-5808, SLIEDREGT et al., “Design and Synthesis of Novel Amphiphilic Dendritic Galactosides for Selective Targeting of Liposomes to the Hepatic Asialoglycoprotein Receptor” J. Med. Chem. (1999) 42:609-618, and Valentijn et al., “Solid-phase synthesis of lysine-based cluster galactosides with high affinity for the Asialoglycoprotein Receptor” Tetrahedron, 1997, 53(2), 759-770, each of which is incorporated by reference herein in its entirety.

In certain embodiments, conjugated antisense compounds comprise an RNase H based oligonucleotide (such as a gapmer) or a splice modulating oligonucleotide (such as a fully modified oligonucleotide) and any conjugate group comprising at least one, two, or three GalNAc groups. In certain embodiments a conjugated antisense compound comprises any conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/01 19724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132; each of which is incorporated by reference in its entirety.

### In Vitro Testing of Antisense Oligonucleotides

Described herein are methods for treatment of cells with antisense oligonucleotides, which can be modified appropriately for treatment with other antisense compounds.

Cells may be treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, CA). Antisense oligonucleotides may be mixed with LIPOFECTIN in OPTI-MEM 1 (Invitrogen, Carlsbad, CA) to achieve the desired final concentration of antisense oligonucleotide and a LIPOFECTIN concentration that may range from 2 to 12 μg/mL per 100 nM antisense oligonucleotide.

Another reagent used to introduce antisense oligonucleotides into cultured cells includes LIPOFECTAMINE (Invitrogen, Carlsbad, CA). Antisense oligonucleotide is mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that may range from 2 to 12 μg/mL per 100 nM antisense oligonucleotide.

Another technique used to introduce antisense oligonucleotides into cultured cells includes electroporation.

Yet another technique used to introduce antisense oligonucleotides into cultured cells includes free uptake of the oligonucleotides by the cells.

Cells are treated with antisense oligonucleotides by routine methods. Cells may be harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

RNA Isolation RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, CA) according to the manufacturer's recommended protocols.

Certain Indications Certain embodiments provided herein relate to methods of treating, preventing, or ameliorating a disease associated with dysregulation of the complement alternative pathway in a subject by administration of a CFB specific inhibitor, such as an antisense compound targeted to CFB.

Examples of renal diseases associated with dysregulation of the complement alternative pathway treatable, preventable, and/or ameliorable with the methods provided herein include C3 glomerulopathy, atypical hemolytic uremic syndrome (aHUS), dense deposit disease (DDD; also known as MPGN Type II or C3Neph), and CFHR5 nephropathy.

Additional renal diseases associated with dysregulation of the complement alternative pathway treatable, preventable, and/or ameliorable with the methods provided herein include IgA nephropathy; mesangiocapillary (membranoproliferative) glomerulonephritis (MPGN); autoimmune disorders including lupus nephritis and systemic lupus erythematosus (SLE); infection-induced glomerulonephritis (also known as Postinfectious glomerulonephritis); and renal ischemia-reperfusion injury, for example post-transplant renal ischemia-reperfusion injury.

Examples of non-renal disorders associated with dysregulation of the complement alternative pathway treatable and/or preventable with the methods provided herein include ocular diseases such as macular degeneration, for example age-related macular degeneration (AMD), including wet AMD and dry AMD, such as Geographic Atrophy; neuromyelitis optica; corneal disease, such as corneal inflammation; autoimmune uveitis; and diabetic retinopathy. It has been reported that complement system is involved in ocular diseases. Jha P, et al., Mol Immunol (2007) 44(16): 3901-3908. Additional examples of non-renal disorders associated with dysregulation of the complement alternative pathway treatable and/or preventable with the methods provided herein include ANCA-associated vasculitis, antiphospholipid syndrome (also known as antiphospholipid antibody syndrome (APS)), asthma, rheumatoid arthritis, Myasthenia Gravis, and multiple sclerosis.

Certain embodiments provided herein relate to methods of treating, preventing, or ameliorating a renal disease associated with dysregulation of the complement alternative pathway in a subject by administration of a CFB specific inhibitor, such as an antisense compound targeted to CFB. In certain aspects, the renal disease is lupus nephritis, systemic lupus erythematosus (SLE), dense deposit disease (DDD), C3 glomerulonephritis (C3GN), CFHR5 nephropathy, or atypical hemolytic uremic syndrome (aHUS), or any combination thereof.

Certain embodiments provided herein relate to methods of treating, preventing, or ameliorating macular degeneration, such as age-related macular degeneration (AMD), in a subject by administration of a CFB specific inhibitor, such as an antisense compound targeted to CFB. In certain aspects, the AMD is wet AMD or dry AMD. In certain aspects, dry AMD can be Geographic Atrophy. Studies have demonstrated the association of complement alternative pathway dysregulation and AMD. Complement components are common constituents of ocular drusen, the extracellular material that accumulates in the macula of AMD patients. Furthermore, it has been reported that CFH and CFB variants account for nearly 75% of AMD cases in northern Europe and North America. It has also been found that a specific CFB polymorphism confers protection against AMD. Patel, N. et al., Eye (2008) 22(6):768-76. Additionally, CFB homozygous null mice have lower complement pathway activity, exhibit smaller ocular lesions, and choroidal neovascularization (CNV) after laser photocoagulation. Rohrer, B. et al., Invest Ophthalmol Vis Sci. (2009) 50(7):3056-64. Furthermore, CFB siRNA treatment protects mice from laser induced CNV. Bora, N S et al., J Immunol. (2006) 177(3):1872-8. Studies have also shown that the kidney and eye share developmental pathways and structural features including basement membrane collagen IV protomer composition and vascularity. Savige et al., J Am Soc Nephrol. (2011) 22(8):1403-15. There is evidence that the complement pathway is involved in renal and ocular diseases. For instance, inherited complement regulatory protein deficiency causes predisposition to atypical hemolytic uremic syndrome and AMD. Richards A et al., Adv Immunol. (2007) 96:141-77. Additionally, chronic kidney disease has been associated with AMD. Nitsch, D. et al., Ophthalmic Epidemiol. (2009) 16(3):181-6; Choi, J. et al, Ophthalmic Epidemiol. (2011) 18(6):259-63. Dense deposit disease (DDD), a kidney disease associated with dysregulated complement alternative pathway, is characterized by acute nephritic syndrome and ocular drusen. Cruz and Smith, GeneReviews (2007) July 20. Moreover, mice harboring genetic deletion of a component of the complement alternative pathway have coexisting renal and ocular disease phenotypes. It has been reported that CFH homozygous null mice develop DDD and present retinal abnormalities and visual dysfunction. Pickering et al., Nat Genet. (2002) 31(4):424-8. Mouse models of renal diseases associated with dysregulation of the complement alternative pathway are also accepted as models of AMD. Pennesi M E et al., Mol Aspects Med (2012) 33:487-509. CFH null mice, for example, are an accepted model for renal diseases, such as DDD, and AMD. Furthermore, it has been reported that AMD is associated with the systemic source of complement factors, which accumulate locally in the eye to drive alternative pathway complement activation. Loyet et al., Invest Ophthalmol Vis Sci. (2012) 53(10):6628-37.

### Examples

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif And, for example, where a particular high-affinity modification appears at a particular position, other high-affinity modifications at the same position are considered suitable, unless otherwise indicated.

### Example 1: General Method for the Preparation of Phosphoramidites, Compounds 1, 1a and 2

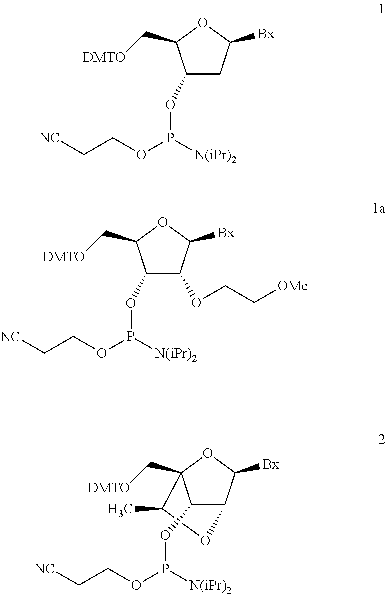

Bx is a heterocyclic base;

Compounds 1, 1a and 2 were prepared as per the procedures well known in the art as described in the specification herein (see Seth et al., Bioorg. Med. Chem., 2011, 21(4), 1122-1125, J. Org. Chem., 2010, 75(5), 1569-1581, Nucleic Acids Symposium Series, 2008, 52(1), 553-554); and also see published PCT International Applications (WO 2011/115818, WO 2010/077578, WO2010/036698, WO2009/143369, WO 2009/006478, and WO 2007/090071), and U.S. Pat. No. 7,569,686).

### Example 2: Preparation of Compound 7

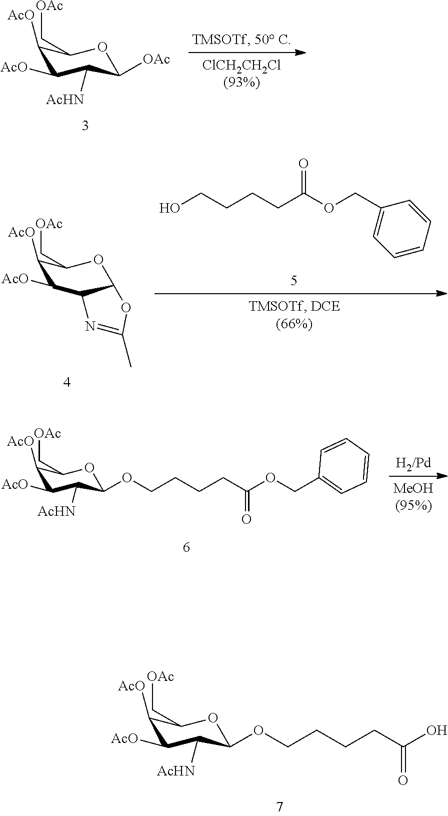

Compounds 3 (2-acetamido-1,3,4,6-tetra-O-acetyl-2-deoxy-β-Dgalactopyranose or galactosamine pentaacetate) is commercially available. Compound 5 was prepared according to published procedures (Weber et al., J. Med. Chem., 1991, 34, 2692).

### Example 3: Preparation of Compound 11

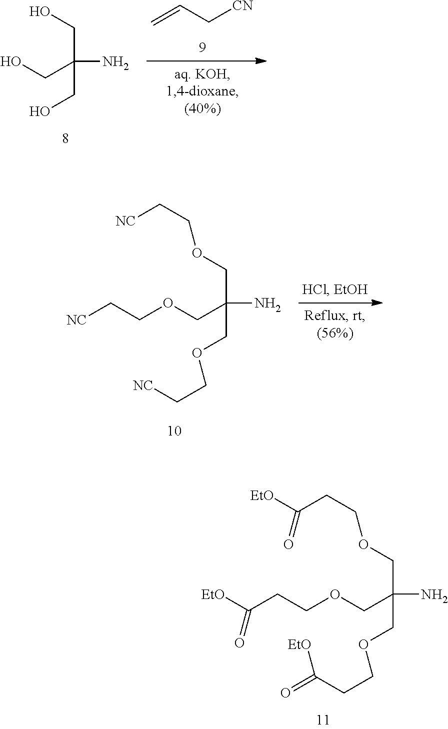

Compounds and 9 are commercially available.

### Example 4: Preparation of Compound 18

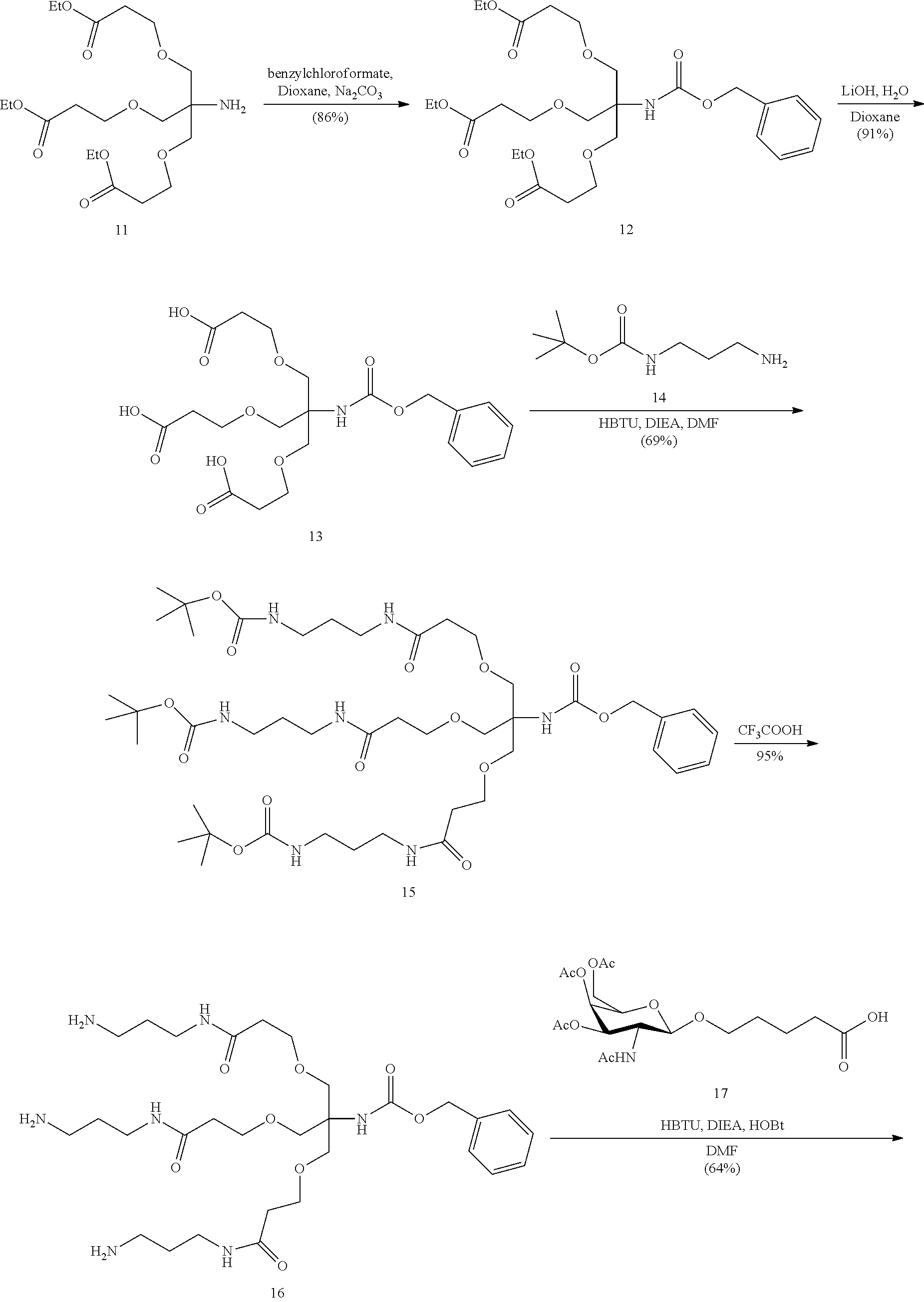

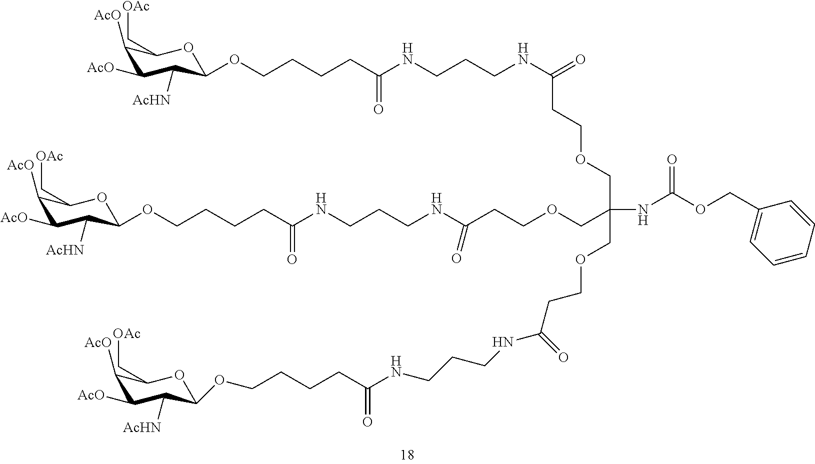

Compound 11 was prepared as per the procedures illustrated in Example 3. Compound 14 is commercially available. Compound 17 was prepared using similar procedures reported by Rensen et al., J. Med. Chem., 2004, 47, 5798-5808.

### Example 5: Preparation of Compound 23

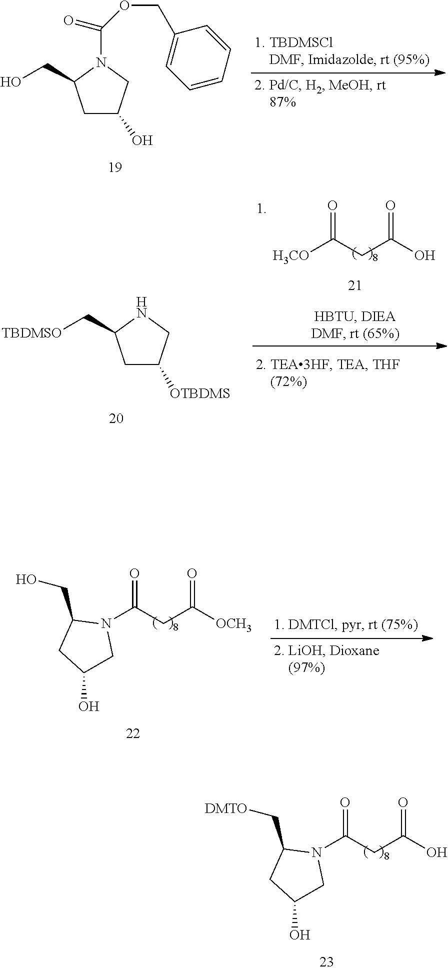

Compounds 19 and 21 are commercially available.

### Example 6: Preparation of Compound 24

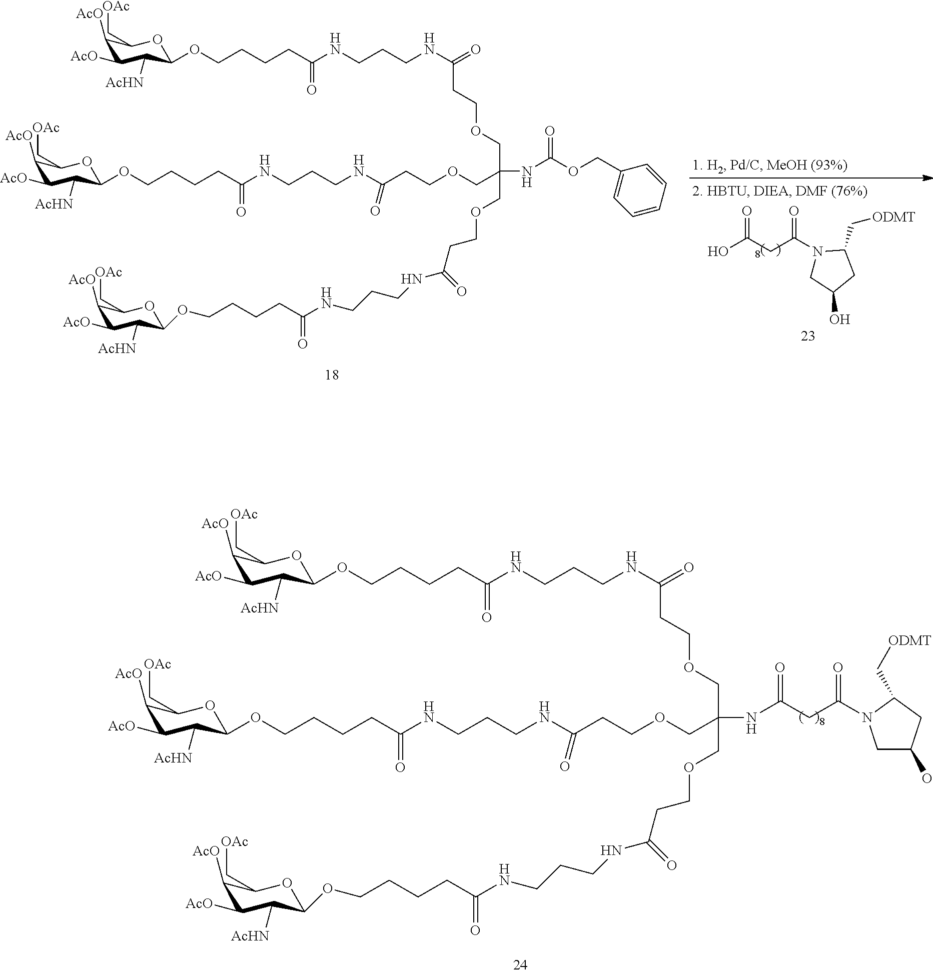

Compounds 18 and 23 were prepared as per the procedures illustrated in Examples 4 and 5.

### Example 7: Preparation of Compound 25

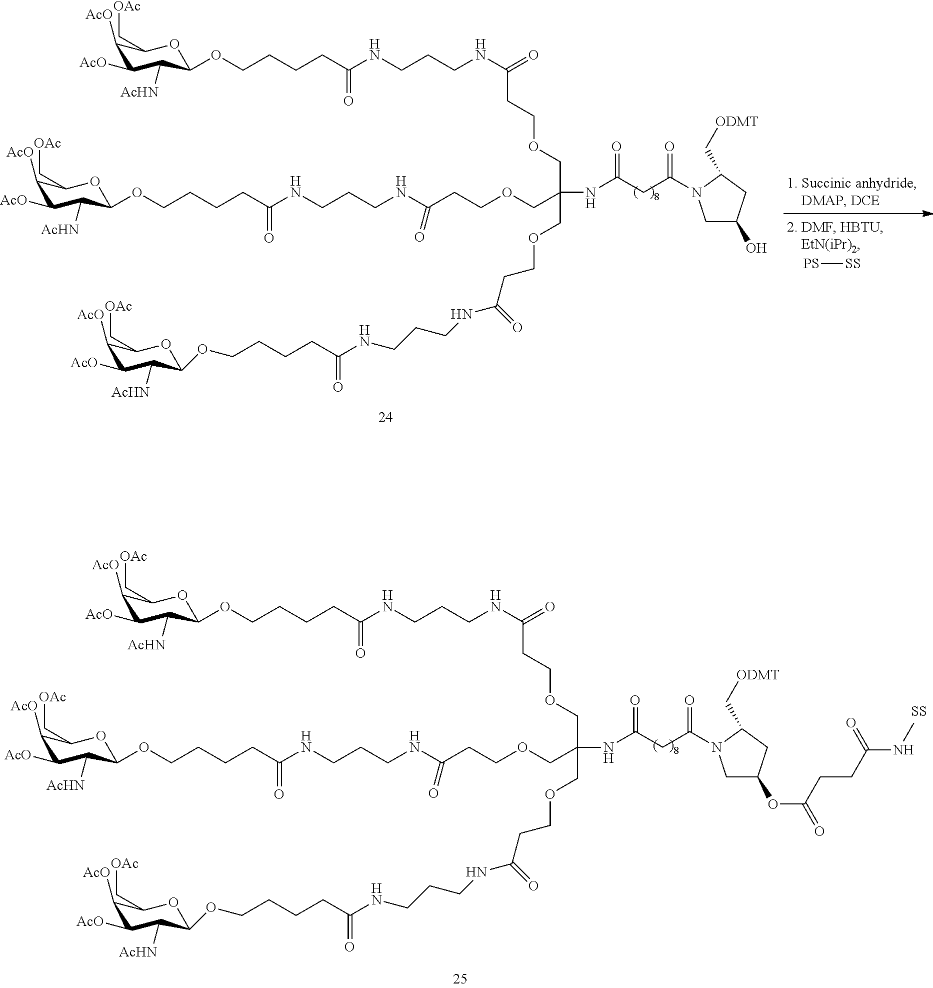

Compound 24 was prepared as per the procedures illustrated in Example 6.

### Example 8: Preparation of Compound 26

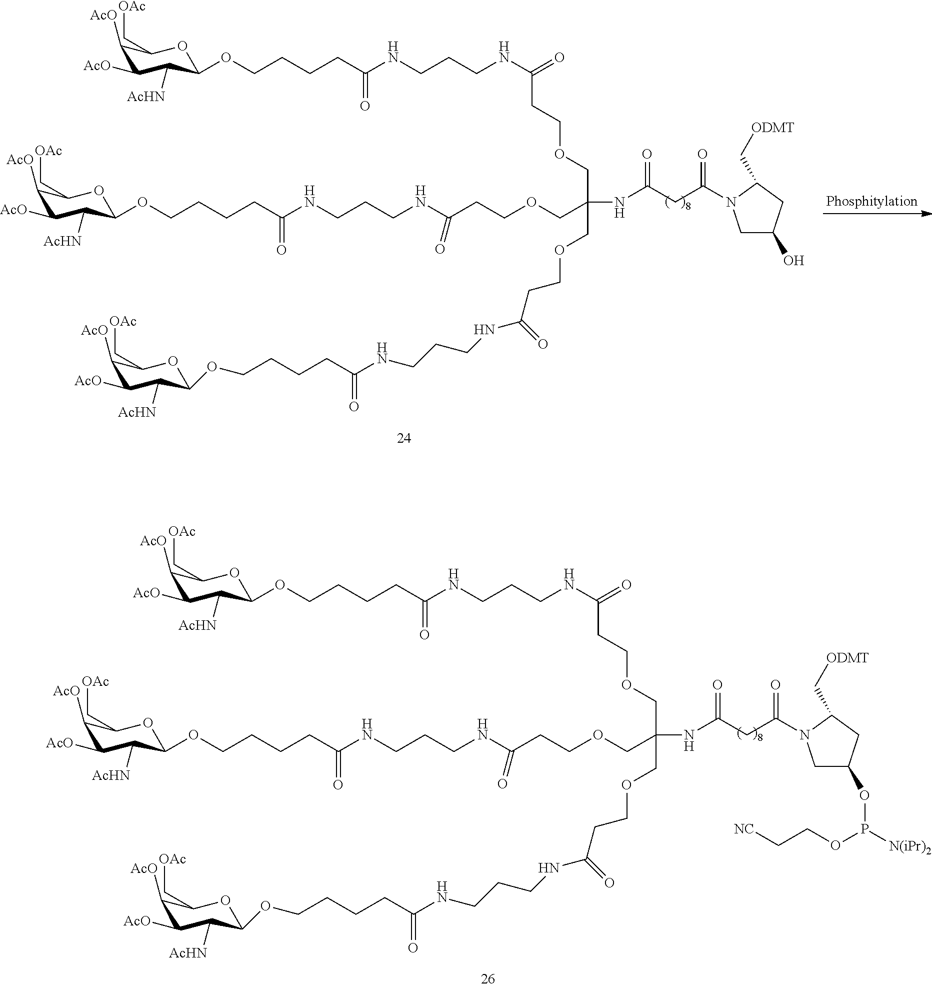

Compound 24 is prepared as per the procedures illustrated in Example 6.

### Example 9: General Preparation of Conjugated ASOs Comprising GalNAc₃-1 at the 3′ Terminus, Compound 29

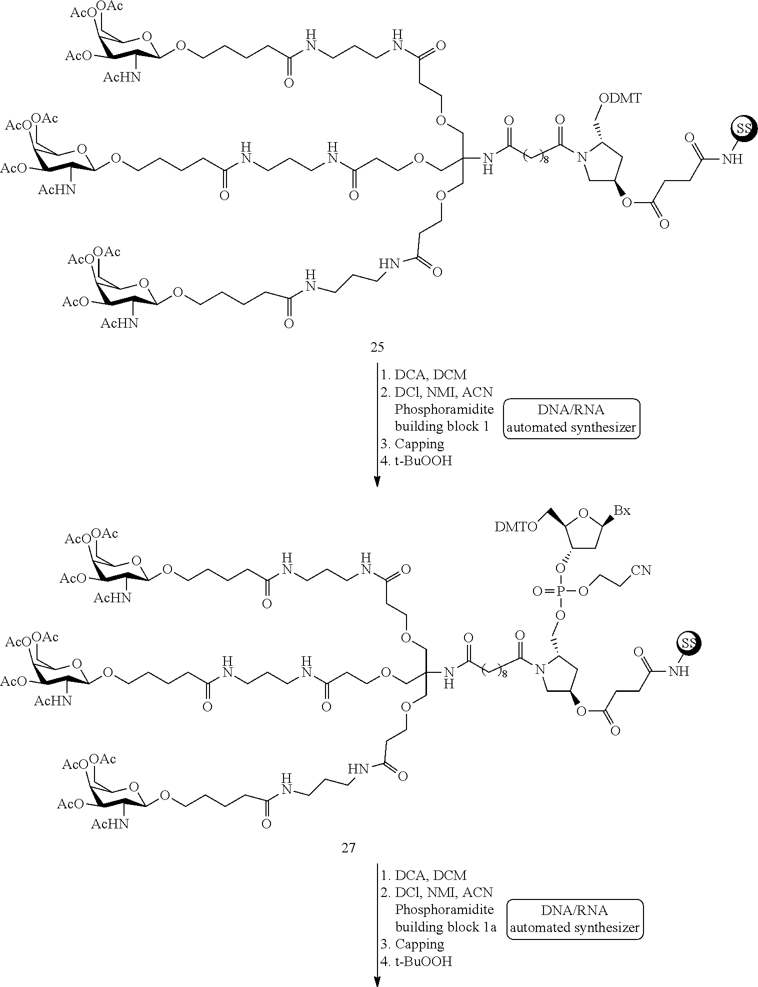

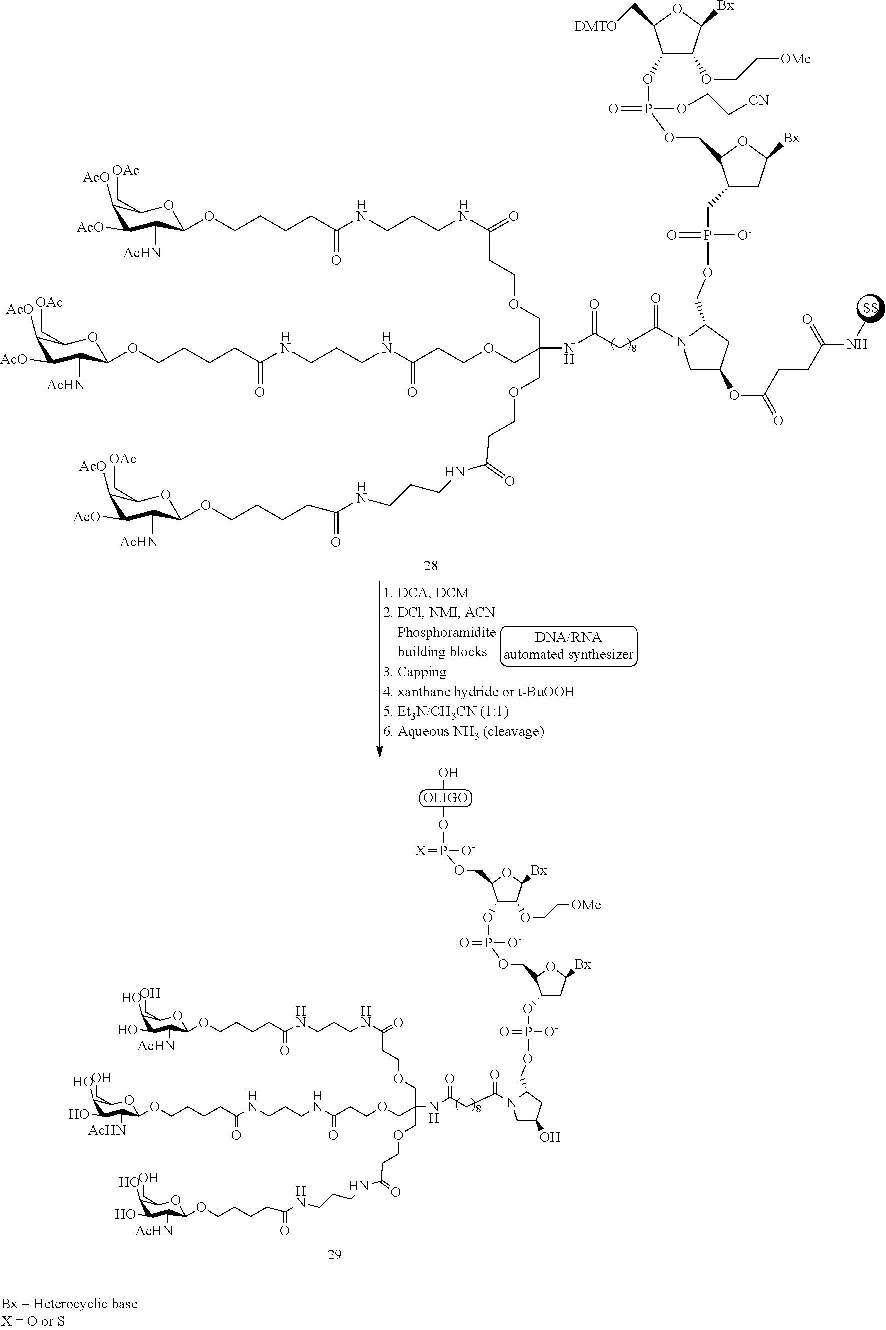

Wherein the protected GalNAc₃-1 has the structure:

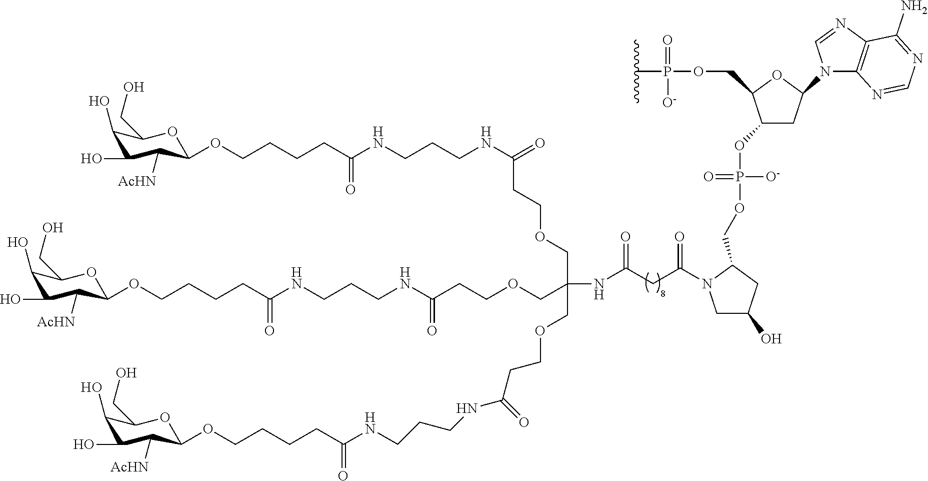

The GalNAc₃ cluster portion of the conjugate group GalNAc₃-1 (GalNAc₃-1ₐ) can be combined with any cleavable moiety to provide a variety of conjugate groups. Wherein GalNAc₃-1ₐ has the formula:

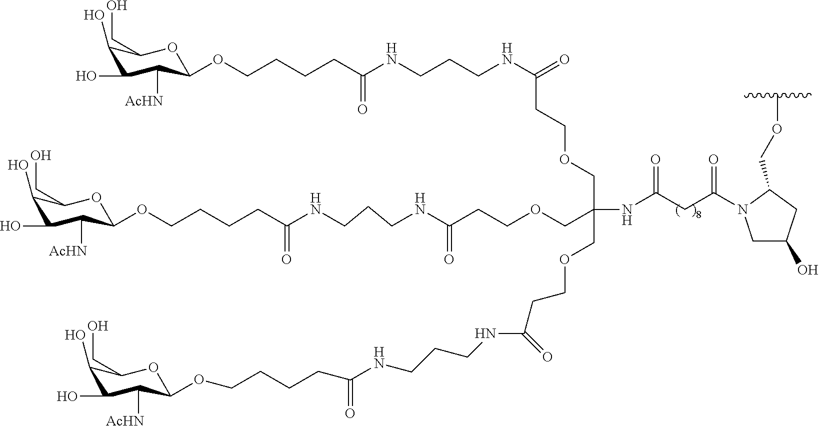

The solid support bound protected GalNAc₃-1, Compound 25, was prepared as per the procedures illustrated in Example 7. Oligomeric Compound 29 comprising GalNAc₃-1 at the 3′ terminus was prepared using standard procedures in automated DNA/RNA synthesis (see Dupouy et al., Angew. Chem. Int. Ed., 2006, 45, 3623-3627). Phosphoramidite building blocks, Compounds 1 and 1a were prepared as per the procedures illustrated in Example 1. The phosphoramidites illustrated are meant to be representative and not intended to be limiting as other phosphoramidite building blocks can be used to prepare oligomeric compounds having a predetermined sequence and composition. The order and quantity of phosphoramidites added to the solid support can be adjusted to prepare gapped oligomeric compounds as described herein. Such gapped oligomeric compounds can have predetermined composition and base sequence as dictated by any given target.

### Example 10: General Preparation Conjugated ASOs Comprising GalNAc₃-1 at the 5′ Terminus, Compound 34

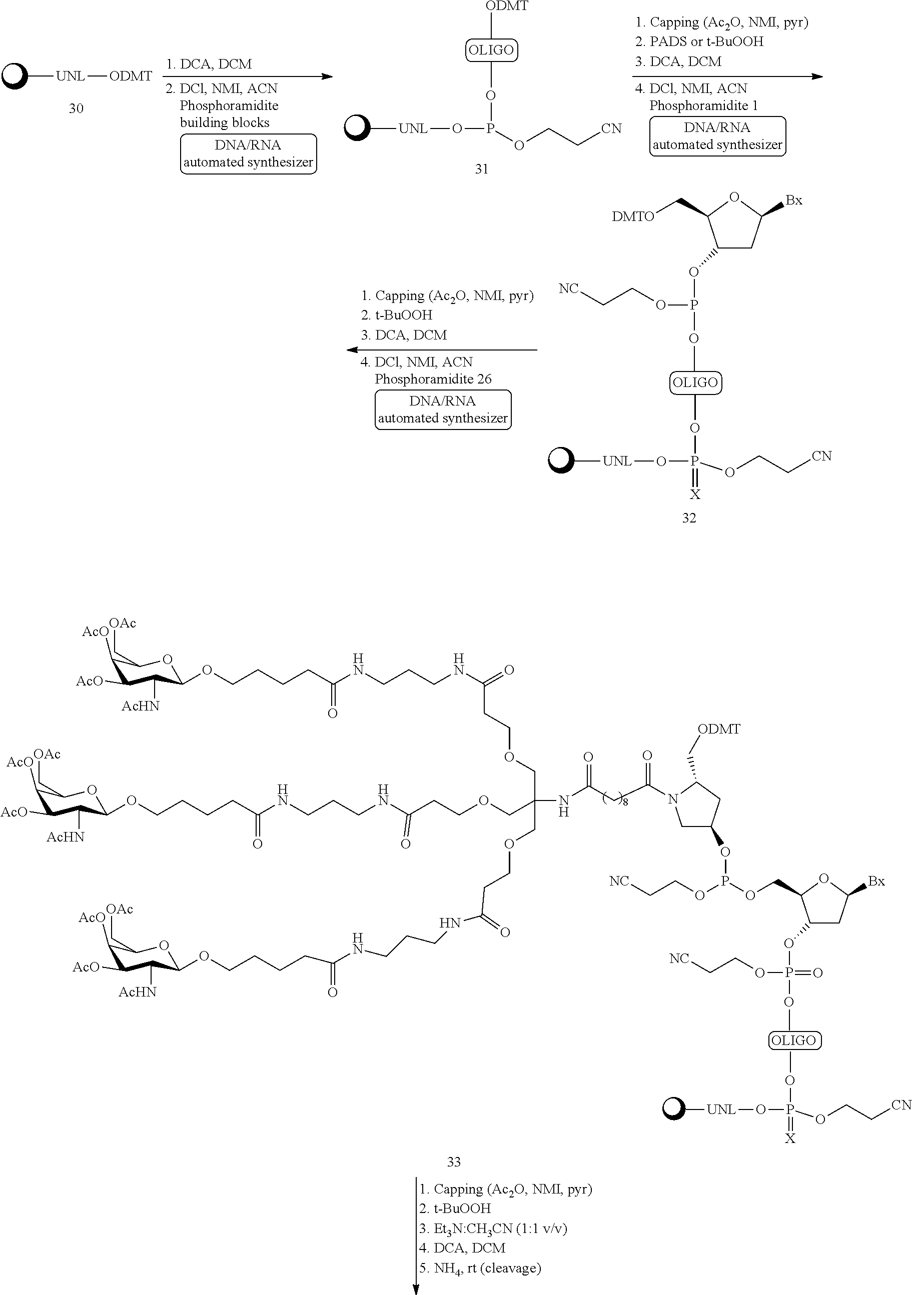

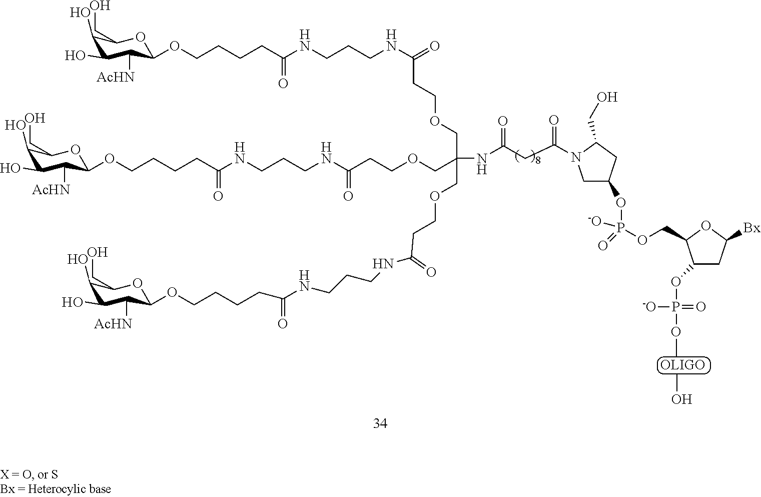

The Unylinker™ 30 is commercially available. Oligomeric Compound 34 comprising a GalNAc₃-1 cluster at the 5′ terminus is prepared using standard procedures in automated DNA/RNA synthesis (see Dupouy et al., Angew. Chem. Int. Ed., 2006, 45, 3623-3627). Phosphoramidite building blocks, Compounds 1 and 1a were prepared as per the procedures illustrated in Example 1. The phosphoramidites illustrated are meant to be representative and not intended to be limiting as other phosphoramidite building blocks can be used to prepare an oligomeric compound having a predetermined sequence and composition. The order and quantity of phosphoramidites added to the solid support can be adjusted to prepare gapped oligomeric compounds as described herein. Such gapped oligomeric compounds can have predetermined composition and base sequence as dictated by any given target.

### Example 11: Preparation of Compound 39

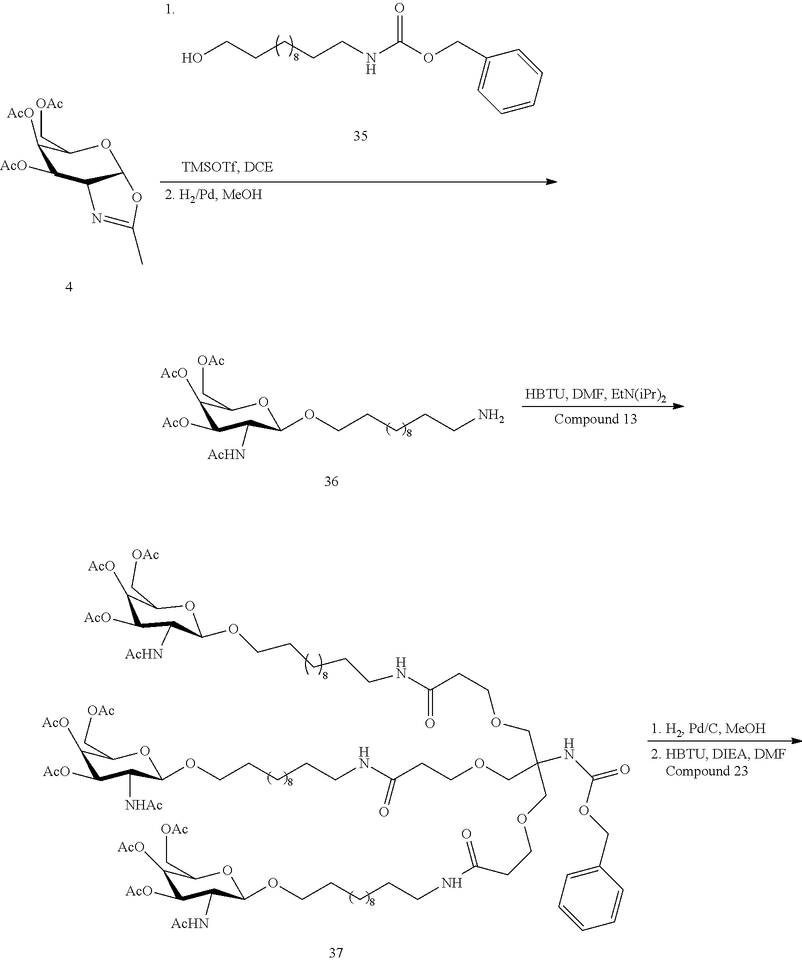

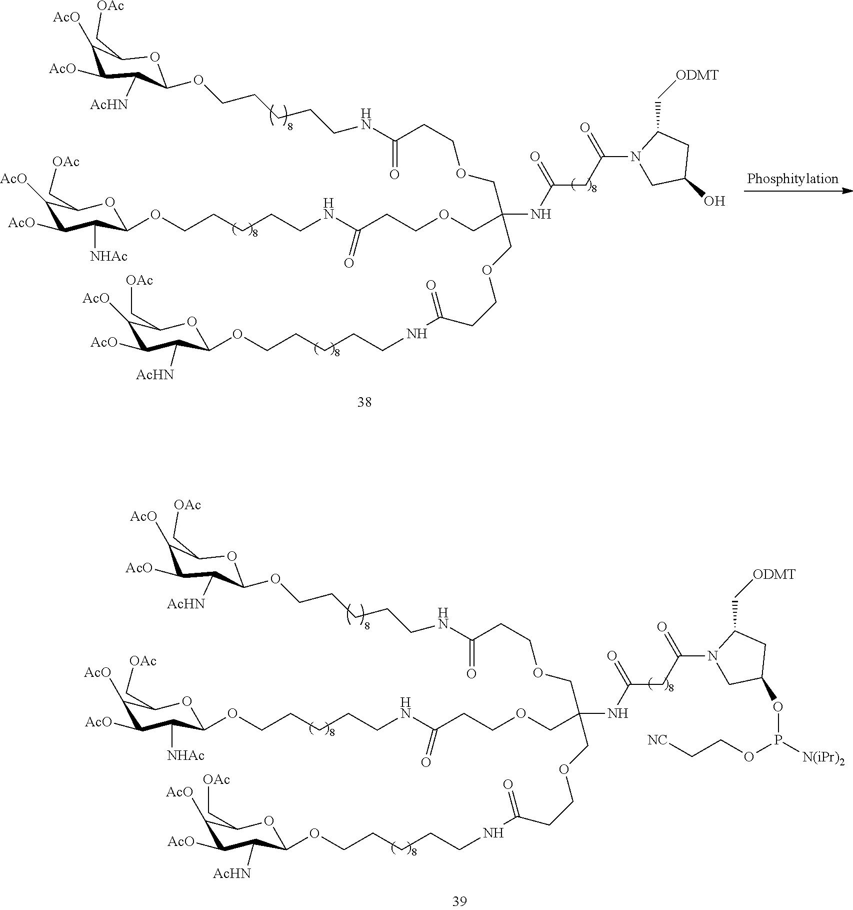

Compounds 4, 13 and 23 were prepared as per the procedures illustrated in Examples 2, 4, and 5. Compound 35 is prepared using similar procedures published in Rouchaud et al., Eur. J Org. Chem., 2011, 12, 2346-2353.

### Example 12: Preparation of Compound 40

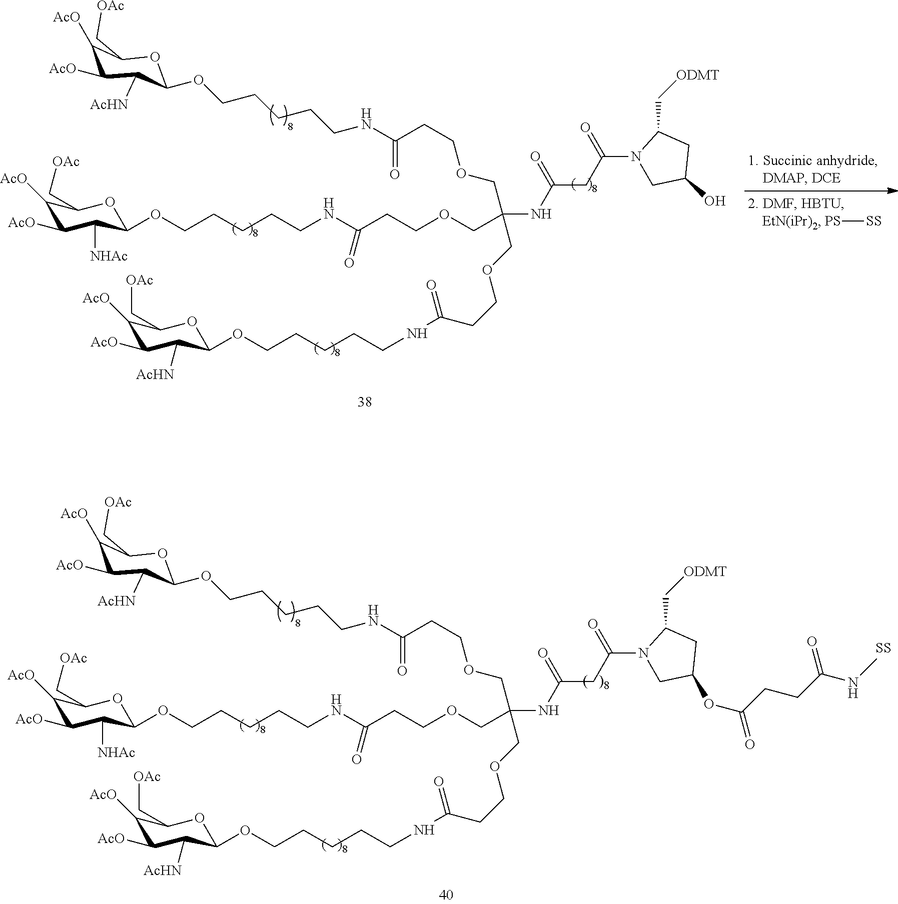

Compound 38 is prepared as per the procedures illustrated in Example 11.

### Example 13: Preparation of Compound 44

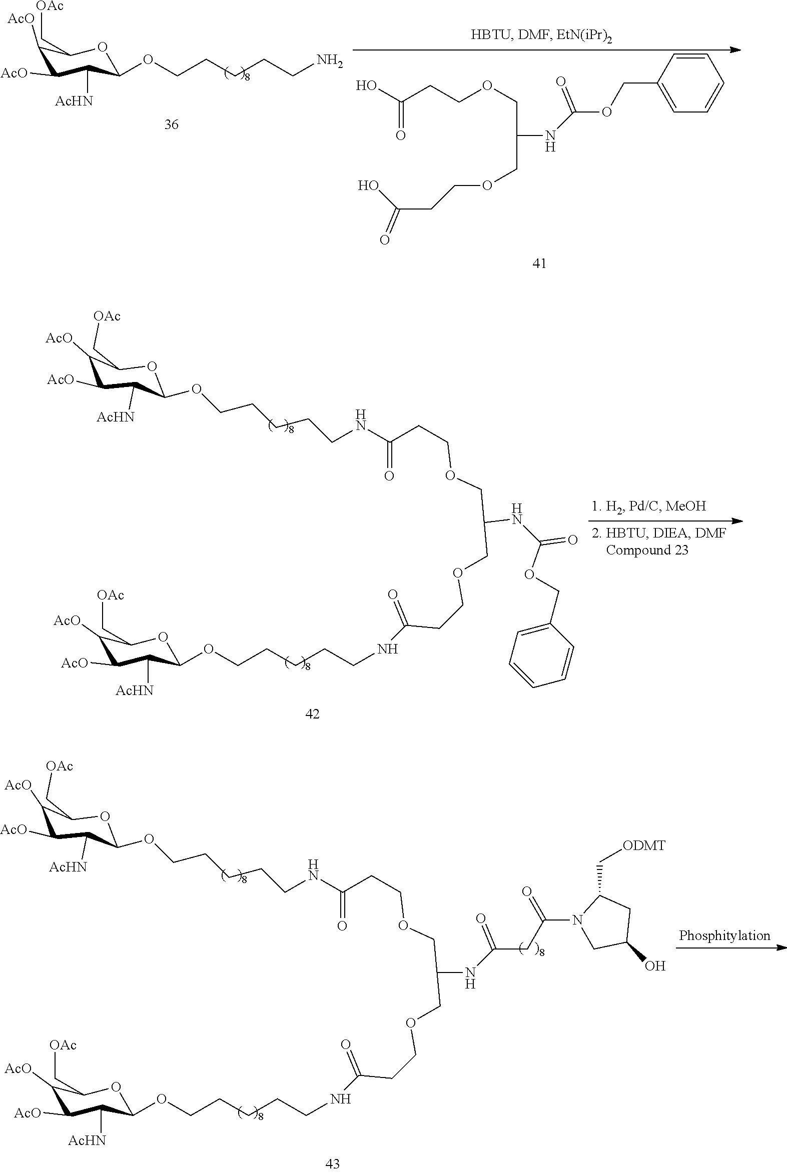

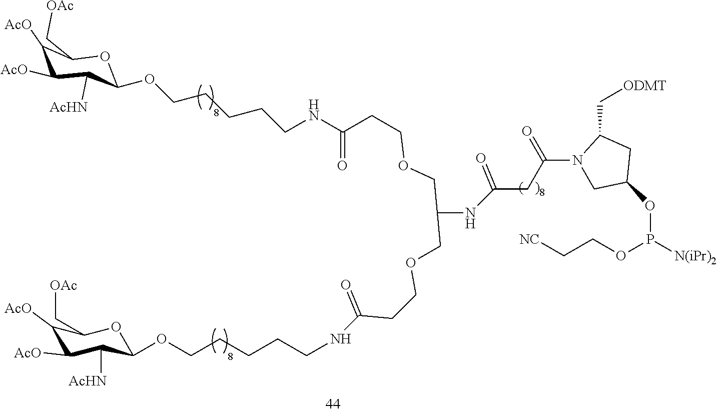

Compounds 23 and 36 are prepared as per the procedures illustrated in Examples 5 and 11. Compound 41 is prepared using similar procedures published in WO 2009082607.

### Example 14: Preparation of Compound 45

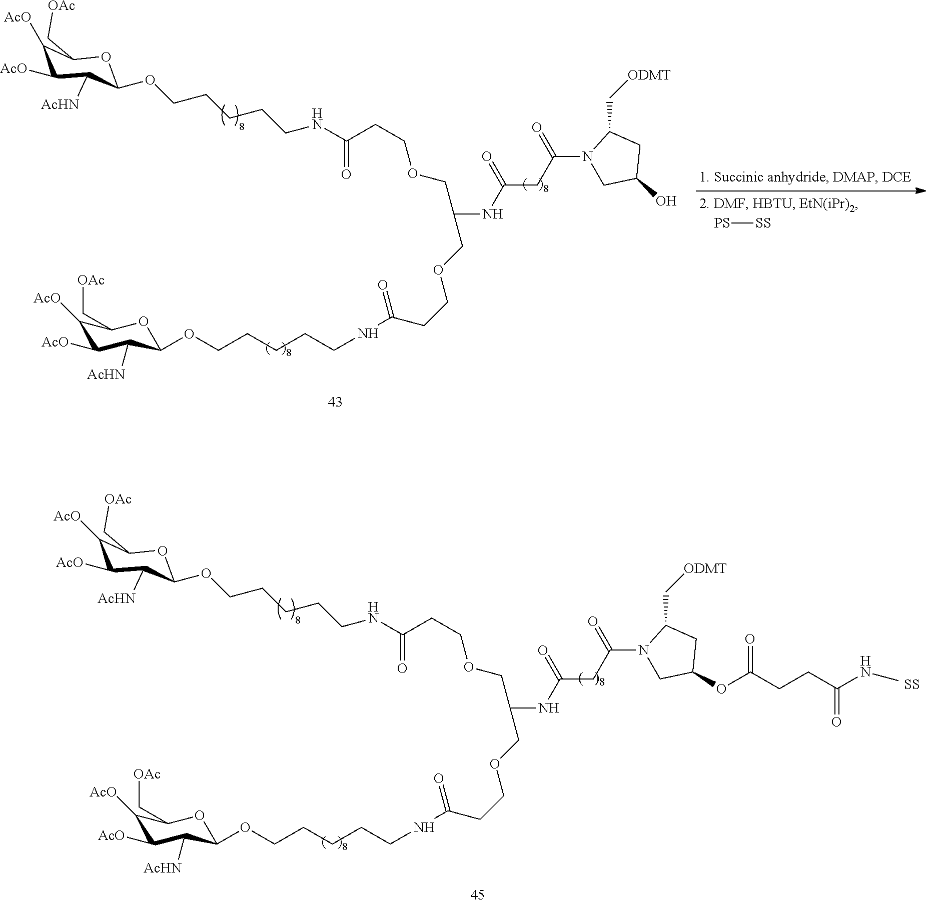

Compound 43 is prepared as per the procedures illustrated in Example 13.

### Example 15: Preparation of Compound 47

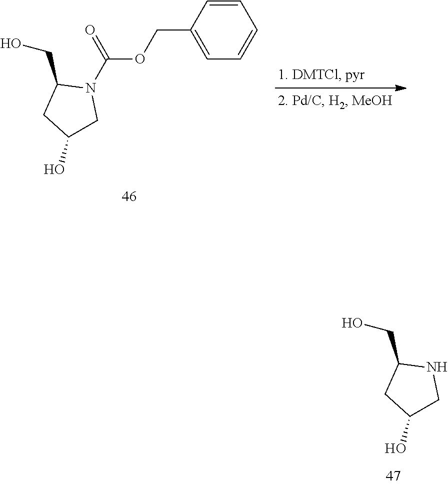 [SMILES: O=C(OCc1ccccc1)N1C[C@H](O)C[C@H]1CO ; OC[C@@H]1C[C@@H](O)CN1]

Compound 46 is commercially available.

### Example 16: Preparation of Compound 53

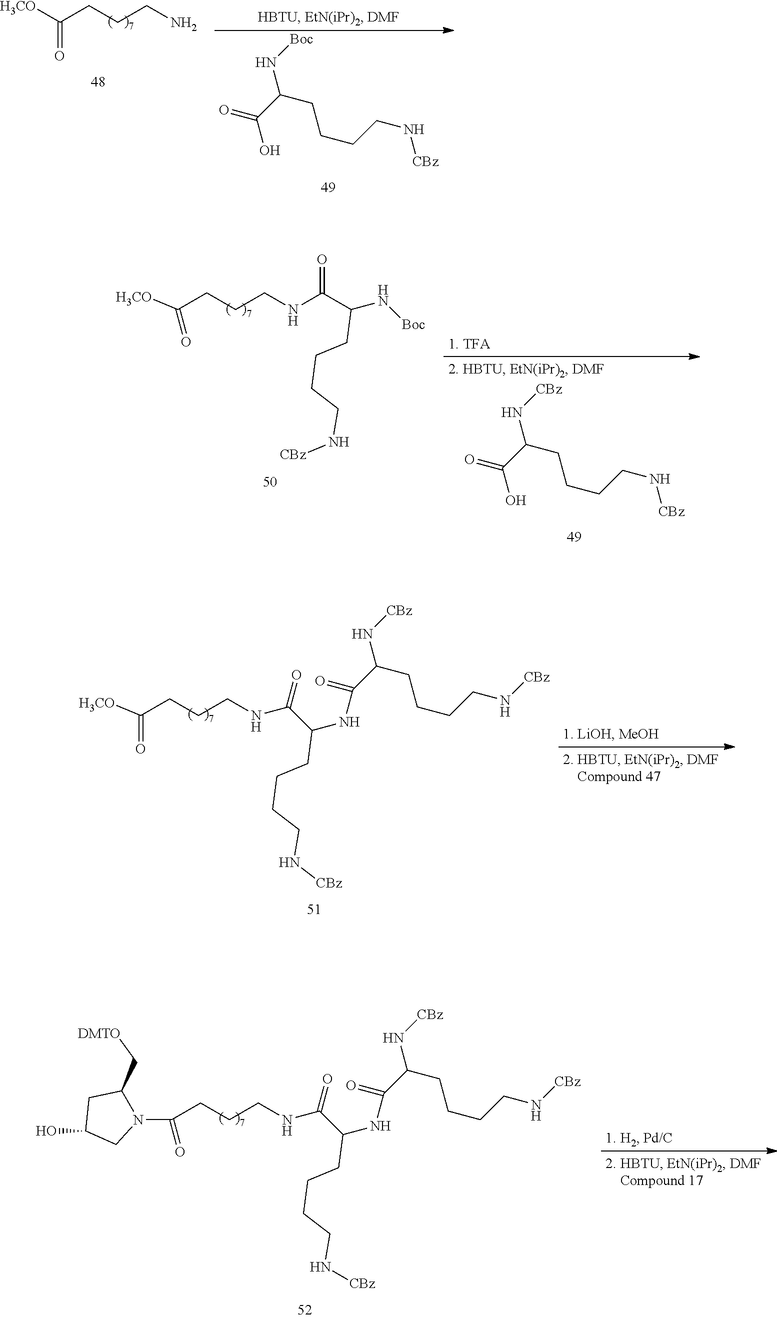

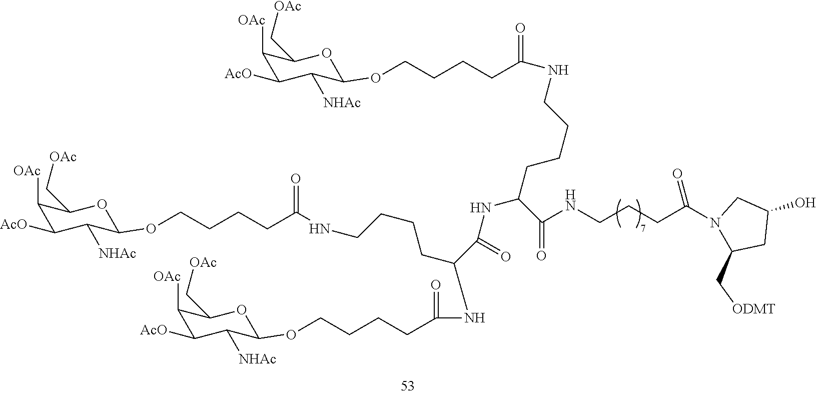

Compounds 48 and 49 are commercially available. Compounds 17 and 47 are prepared as per the procedures illustrated in Examples 4 and 15.

### Example 17: Preparation of Compound 54

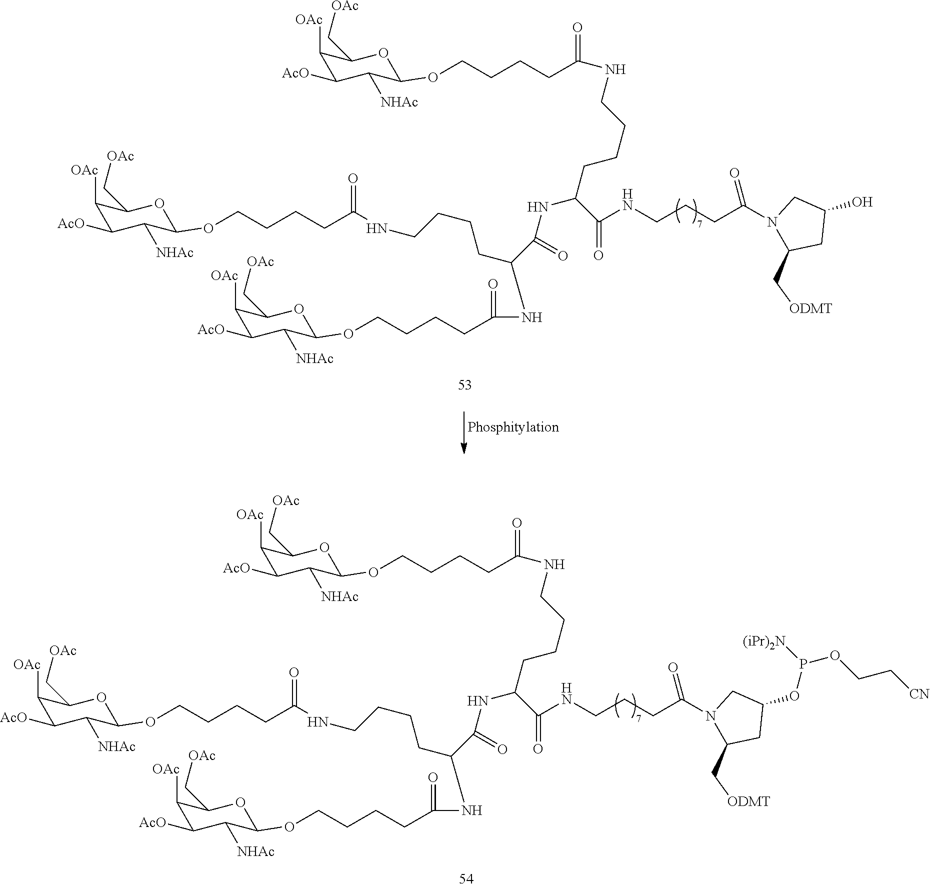

Compound 53 is prepared as per the procedures illustrated in Example 16.

### Example 18: Preparation of Compound 55

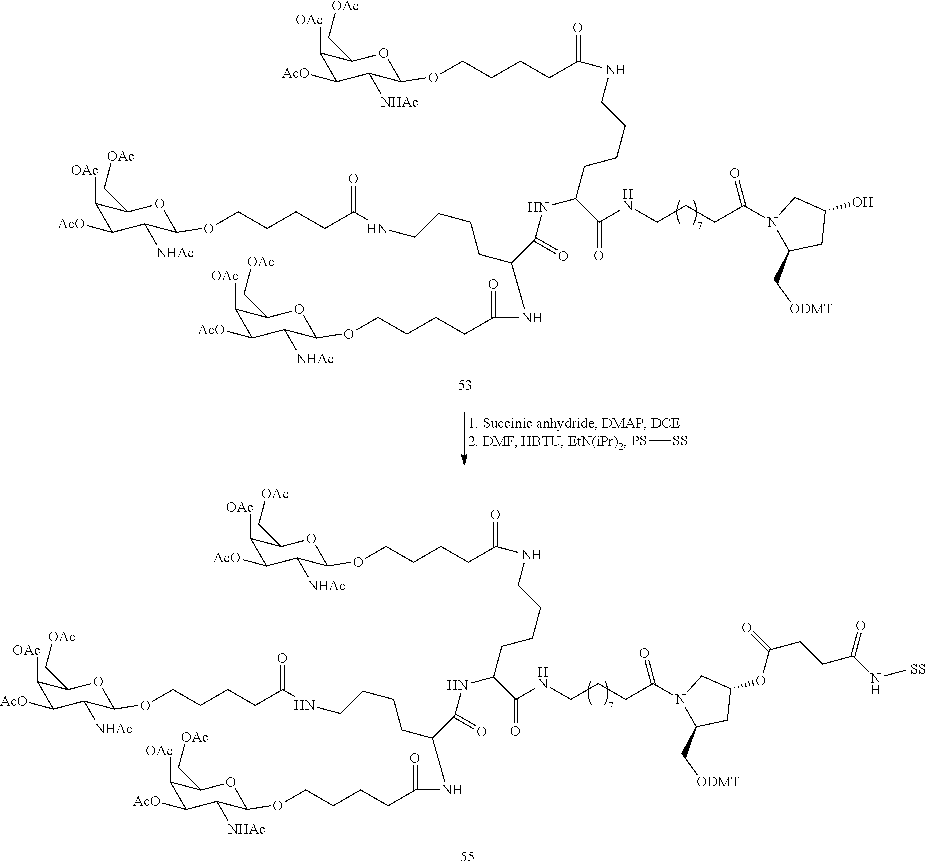

Compound 53 is prepared as per the procedures illustrated in Example 16.

### Example 19: General Method for the Preparation of Conjugated ASOs Comprising GalNAc₃-1 at the 3′ Position Via Solid Phase Techniques (Preparation of ISIS 647535, 647536 and 651900)

Unless otherwise stated, all reagents and solutions used for the synthesis of oligomeric compounds are purchased from commercial sources. Standard phosphoramidite building blocks and solid support are used for incorporation nucleoside residues which include for example T, A, G, and ^{m}C residues. A 0.1 M solution of phosphoramidite in anhydrous acetonitrile was used for β-D-2′-deoxyribonucleoside and 2′-MOE.

The ASO syntheses were performed on ABI 394 synthesizer (1-2 μmol scale) or on GE Healthcare Bioscience ÄKTA oligopilot synthesizer (40-200 μmol scale) by the phosphoramidite coupling method on an GalNAc₃-1 loaded VIMAD solid support (110 μmol/g, Guzaev et al., 2003) packed in the column. For the coupling step, the phosphoramidites were delivered 4 fold excess over the loading on the solid support and phosphoramidite condensation was carried out for 10 min. All other steps followed standard protocols supplied by the manufacturer. A solution of 6% dichloroacetic acid in toluene was used for removing dimethoxytrityl (DMT) group from 5′-hydroxyl group of the nucleotide. 4,5-Dicyanoimidazole (0.7 M) in anhydrous CH₃CN was used as activator during coupling step. Phosphorothioate linkages were introduced by sulfurization with 0.1 M solution of xanthane hydride in 1:1 pyridine/CH3CN for a contact time of 3 minutes. A solution of 20% tert-butylhydroperoxide in CH3CN containing 6% water was used as an oxidizing agent to provide phosphodiester internucleoside linkages with a contact time of 12 minutes.

After the desired sequence was assembled, the cyanoethyl phosphate protecting groups were deprotected using a 1:1 (v/v) mixture of triethylamine and acetonitrile with a contact time of 45 minutes. The solid-support bound ASOs were suspended in aqueous ammonia (28-30 wt %) and heated at 55° C. for 6 h.

The unbound ASOs were then filtered and the ammonia was boiled off. The residue was purified by high pressure liquid chromatography on a strong anion exchange column (GE Healthcare Bioscience, Source 30Q, 30 μm, 2.54×8 cm, A=100 mM ammonium acetate in 30% aqueous CH3CN, B=1.5 M NaBr in A, 0-40% of B in 60 min, flow 14 mL min-1, λ=260 nm). The residue was desalted by HPLC on a reverse phase column to yield the desired ASOs in an isolated yield of 15-30% based on the initial loading on the solid support. The ASOs were characterized by ion-pair-HPLC coupled MS analysis with Agilent 1100 MSD system.

Antisense oligonucleotides not comprising a conjugate were synthesized using standard oligonucleotide synthesis procedures well known in the art.

Using these methods, three separate antisense compounds targeting ApoC III were prepared. As summarized in Table 17, below, each of the three antisense compounds targeting ApoC III had the same nucleobase sequence; ISIS 304801 is a 5-10-5 MOE gapmer having all phosphorothioate linkages; ISIS 647535 is the same as ISIS 304801, except that it had a GalNAc₃-1 conjugated at its 3′end; and ISIS 647536 is the same as ISIS 647535 except that certain internucleoside linkages of that compound are phosphodiester linkages. As further summarized in Table 17, two separate antisense compounds targeting SRB-1 were synthesized. ISIS 440762 was a 2-10-2 cEt gapmer with all phosphorothioate internucleoside linkages; ISIS 651900 is the same as ISIS 440762, except that it included a GalNAc₃-1 at its 3′-end.

TABLE 17
Modified ASO targeting ApoC III and SRB-1

| | | | | | SEQ |
|---|---|---|---|---|---|
| | | | CalCd | Observed | ID |
| ASO | Sequence (5′ to 3′) | Target | Mass | Mass | No. |
| ISIS | AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds} TₑₛTₑₛTₑₛAₑₛTₑ | ApoC | 7165.4 | 7164.4 | 821 |
| 304801 | | III | | | |
| ISIS | AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛTₑₛAₑₛTₑₒA_{do′}- | ApoC | 9239.5 | 9237.8 | 822 |
| 647535 | GalNAc₃-1ₐ | III | | | |
| ISIS | AₑₛGₑₒ^{m}CₑₒTₑₒTₑₒ^{m}C_{ds}T_{ds}T_{ds}G_{ds}T_{ds}^{m}C_{ds}^{m}C_{ds}A_{ds}G_{ds}^{m}C_{ds}TₑₒTₑₒTₑₛAₑₛTₑₒA_{do′}- | ApoC | 9142.9 | 9140.8 | 822 |
| 647536 | GalNAc₃-1ₐ | III | | | |
| ISIS | Tₖₛ^{m}CₖₛA_{ds}G_{ds}T_{ds}^{m}C_{ds}A_{ds}T_{ds}G_{ds}A_{ds}^{m}C_{ds}T_{ds}Tₖₛ^{m}Cₖ | SRB-1 | 4647.0 | 4646.4 | 823 |
| 440762 | | | | | |
| ISIS | Tₖₛ^{m}CₖₛA_{ds}G_{ds}T_{ds}^{m}C_{ds}A_{ds}T_{ds}G_{ds}A_{ds}^{m}C_{ds}T_{ds}Tₖₛ^{m}CₖₒA_{do′}-GalNAc₃-1ₐ | SRB-1 | 6721.1 | 6719.4 | 824 |
| 651900 | | | | | |

Subscripts: “e” indicates 2′-MOE modified nucleoside; “d” indicates β-D-2′-deoxyribonucleoside; “k” indicates 6′-(S)—CH3 bicyclic nucleoside (e.g. cEt); “s” indicates phosphorothioate internucleoside linkages (PS); “o” indicates phosphodiester internucleoside linkages (PO); and “o′” indicates —O—P(═O)(OH)—. Superscript “m” indicates 5-methylcytosines. “GalNAc₃-1” indicates a conjugate group having the structure shown previously in Example 9. Note that GalNAc₃-1 comprises a cleavable adenosine which links the ASO to remainder of the conjugate, which is designated “GalNAc₃-1ₐ.” This nomenclature is used in the above table to show the full nucleobase sequence, including the adenosine, which is part of the conjugate. Thus, in the above table, the sequences could also be listed as ending with “GalNAc₃-1” with the “A_{do}” omitted. This convention of using the subscript “a” to indicate the portion of a conjugate group lacking a cleavable nucleoside or cleavable moiety is used throughout these Examples. This portion of a conjugate group lacking the cleavable moiety is referred to herein as a “cluster” or “conjugate cluster” or “GalNAc3 cluster.” In certain instances it is convenient to describe a conjugate group by separately providing its cluster and its cleavable moiety.

### Example 20: Dose-Dependent Antisense Inhibition of Human ApoC III in huApoC III Transgenic Mice

ISIS 304801 and ISIS 647535, each targeting human ApoC III and described above, were separately tested and evaluated in a dose-dependent study for their ability to inhibit human ApoC III in human ApoC III transgenic mice.

### Treatment

Human ApoCIII transgenic mice were maintained on a 12-hour light/dark cycle and fed ad libitum Teklad lab chow. Animals were acclimated for at least 7 days in the research facility before initiation of the experiment. ASOs were prepared in PBS and sterilized by filtering through a 0.2 micron filter. ASOs were dissolved in 0.9% PBS for injection.

Human ApoC III transgenic mice were injected intraperitoneally once a week for two weeks with ISIS 304801 or 647535 at 0.08, 0.25. 0.75, 2.25 or 6.75 μmol/kg or with PBS as a control. Each treatment group consisted of 4 animals. Forty-eight hours after the administration of the last dose, blood was drawn from each mouse and the mice were sacrificed and tissues were collected.

ApoC III mRNA Analysis

ApoC III mRNA levels in the mice's livers were determined using real-time PCR and RIBOGREEN® RNA quantification reagent (Molecular Probes, Inc. Eugene, OR) according to standard protocols. ApoC III mRNA levels were determined relative to total RNA (using Ribogreen), prior to normalization to PBS-treated control. The results below are presented as the average percent of ApoC III mRNA levels for each treatment group, normalized to PBS-treated control and are denoted as “% PBS”. The half maximal effective dosage (ED₅₀) of each ASO is also presented in Table 18, below.

As illustrated, both antisense compounds reduced ApoC III RNA relative to the PBS control. Further, the antisense compound conjugated to GalNAc₃-1 (ISIS 647535) was substantially more potent than the antisense compound lacking the GalNAc₃-1 conjugate (ISIS 304801).

TABLE 18
Effect of ASO treatment on ApoC III mRNA levels in human ApoC III transgenic mice

| | Dose | % | ED₅₀ | | Internucleoside | SEQ |
|---|---|---|---|---|---|---|
| ASO | (μmol/kg) | PBS | (μmol/kg) | 3′ Conjugate | linkage/Length | ID No. |
| PBS | 0 | 100 | — | — | — | |
| ISIS | 0.08 | 95 | 0.77 | None | PS/20 | 821 |
| 304801 | 0.75 | 42 | | | | |
| | 2.25 | 32 | | | | |
| | 6.75 | 19 | | | | |
| ISIS | 0.08 | 50 | 0.074 | GalNAc₃-1 | PS/20 | 822 |
| 647535 | 0.75 | 15 | | | | |
| | 2.25 | 17 | | | | |
| | 6.75 | 8 | | | | |

### ApoC III Protein Analysis (Turbidometric Assay)

Plasma ApoC III protein analysis was determined using procedures reported by Graham et al, Circulation Research, published online before print Mar. 29, 2013.

Approximately 100 μl of plasma isolated from mice was analyzed without dilution using an Olympus Clinical Analyzer and a commercially available turbidometric ApoC III assay (Kamiya, Cat #KAI-006, Kamiya Biomedical, Seattle, WA). The assay protocol was performed as described by the vendor.

As shown in the Table 19 below, both antisense compounds reduced ApoC III protein relative to the PBS control. Further, the antisense compound conjugated to GalNAc₃-1 (ISIS 647535) was substantially more potent than the antisense compound lacking the GalNAc₃-1 conjugate (ISIS 304801).

TABLE 19
Effect of ASO treatment on ApoC III plasma protein levels in human ApoC III transgenic mice

| | Dose | % | ED₅₀ | | Internucleoside | SEQ |
|---|---|---|---|---|---|---|
| ASO | (μmol/kg) | PBS | (μmol/kg) | 3′ Conjugate | Linkage/Length | ID No. |
| PBS | 0 | 100 | — | — | — | |
| ISIS | 0.08 | 86 | 0.73 | None | PS/20 | 821 |
| 304801 | 0.75 | 51 | | | | |
| | 2.25 | 23 | | | | |
| | 6.75 | 13 | | | | |
| ISIS | 0.08 | 72 | 0.19 | GalNAc₃-1 | PS/20 | 822 |
| 647535 | 0.75 | 14 | | | | |
| | 2.25 | 12 | | | | |
| | 6.75 | 11 | | | | |

Plasma triglycerides and cholesterol were extracted by the method of Bligh and Dyer (Bligh, E. G. and Dyer, W. J. Can. J. Biochem. Physiol. 37: 911-917, 1959)(Bligh, E and Dyer, W, Can J Biochem Physiol, 37, 911-917, 1959)(Bligh, E and Dyer, W, Can J Biochem Physiol, 37, 911-917, 1959) and measured by using a Beckmann Coulter clinical analyzer and commercially available reagents.

The triglyceride levels were measured relative to PBS injected mice and are denoted as “% PBS”. Results are presented in Table 20. As illustrated, both antisense compounds lowered triglyceride levels. Further, the antisense compound conjugated to GalNAc₃-1 (ISIS 647535) was substantially more potent than the antisense compound lacking the GalNAc₃-1 conjugate (ISIS 304801).

TABLE 20
Effect of ASO treatment on triglyceride levels in transgenic mice

| | | | ED₅₀ | | Internucleoside | |
|---|---|---|---|---|---|---|
| | Dose | % | (μmol/ | 3′ | Linkage/ | SEQ |
| ASO | (μmol/kg) | PBS | kg) | Conjugate | Length | ID No. |
| PBS | 0 | 100 | — | — | — | |
| ISIS | 0.08 | 87 | 0.63 | None | PS/20 | 821 |
| 304801 | 0.75 | 46 | | | | |
| | 2.25 | 21 | | | | |
| | 6.75 | 12 | | | | |
| ISIS | 0.08 | 65 | 0.13 | GalNAc₃-1 | PS/20 | 822 |
| 647535 | 0.75 | 9 | | | | |
| | 2.25 | 8 | | | | |
| | 6.75 | 9 | | | | |

Plasma samples were analyzed by HPLC to determine the amount of total cholesterol and of different fractions of cholesterol (HDL and LDL). Results are presented in Tables 21 and 22. As illustrated, both antisense compounds lowered total cholesterol levels; both lowered LDL; and both raised HDL. Further, the antisense compound conjugated to GalNAc₃-1 (ISIS 647535) was substantially more potent than the antisense compound lacking the GalNAc₃-1 conjugate (ISIS 304801). An increase in HDL and a decrease in LDL levels is a cardiovascular beneficial effect of antisense inhibition of ApoC III.

TABLE 21
Effect of ASO treatment on total cholesterol
levels in transgenic mice

| | | | | Inter- | |
|---|---|---|---|---|---|
| | | Total | | nucleoside | |
| | Dose | Cholesterol | 3′ | Linkage/ | SEQ |
| ASO | (μmol/kg) | (mg/dL) | Conjugate | Length | ID No. |
| PBS | 0 | 257 | — | — | |
| ISIS | 0.08 | 226 | None | PS/20 | 821 |
| 304801 | 0.75 | 164 | | | |
| | 2.25 | 110 | | | |
| | 6.75 | 82 | | | |
| ISIS | 0.08 | 230 | GalNAc3-1 | PS/20 | 822 |
| 647535 | 0.75 | 82 | | | |
| | 2.25 | 86 | | | |
| | 6.75 | 99 | | | |

TABLE 22
Effect of ASO treatment on HDL and LDL cholesterol
levels in transgenic mice

| | Dose | HDL | LDL | | Internucleoside | |
|---|---|---|---|---|---|---|
| | (μmol/ | (mg/ | (mg/ | 3′ | Linkage/ | SEQ |
| ASO | kg) | dL) | dL) | Conjugate | Length | ID No. |
| PBS | 0 | 17 | 28 | — | — | |
| ISIS | 0.08 | 17 | 23 | None | PS/20 | 821 |
| 304801 | 0.75 | 27 | 12 | | | |
| | 2.25 | 50 | 4 | | | |
| | 6.75 | 45 | 2 | | | |
| ISIS | 0.08 | 21 | 21 | GalNAc₃-1 | PS/20 | 822 |
| 647535 | 0.75 | 44 | 2 | | | |
| | 2.25 | 50 | 2 | | | |
| | 6.75 | 58 | 2 | | | |

### Pharmacokinetics Analysis (PK)

The PK of the ASOs was also evaluated. Liver and kidney samples were minced and extracted using standard protocols. Samples were analyzed on MSD1 utilizing IP-HPLC-MS. The tissue level (μg/g) of full-length ISIS 304801 and 647535 was measured and the results are provided in Table 23. As illustrated, liver concentrations of total full-length antisense compounds were similar for the two antisense compounds. Thus, even though the GalNAc₃-1-conjugated antisense compound is more active in the liver (as demonstrated by the RNA and protein data above), it is not present at substantially higher concentration in the liver. Indeed, the calculated EC₅₀ (provided in Table 23) confirms that the observed increase in potency of the conjugated compound cannot be entirely attributed to increased accumulation. This result suggests that the conjugate improved potency by a mechanism other than liver accumulation alone, possibly by improving the productive uptake of the antisense compound into cells.

The results also show that the concentration of GalNAc₃-1 conjugated antisense compound in the kidney is lower than that of antisense compound lacking the GalNAc conjugate. This has several beneficial therapeutic implications. For therapeutic indications where activity in the kidney is not sought, exposure to kidney risks kidney toxicity without corresponding benefit. Moreover, high concentration in kidney typically results in loss of compound to the urine resulting in faster clearance.

Accordingly, for non-kidney targets, kidney accumulation is undesired. These data suggest that GalNAc₃-1 conjugation reduces kidney accumulation.

TABLE 23
PK analysis of ASO treatment in transgenic mice

| | | | | | | Inter- | |
|---|---|---|---|---|---|---|---|
| | Dose | | | Liver | | nucleoside | SEQ |
| | (μmol/ | Liver | Kidney | EC₅₀ | 3′ | Linkage/ | ID |
| ASO | kg) | (μg/g) | (μg/g) | (μg/g) | Conjugate | Length | No. |
| ISIS | 0.1 | 5.2 | 2.1 | 53 | None | PS/20 | 821 |
| 304801 | 0.8 | 62.8 | 119.6 | | | | |
| | 2.3 | 142.3 | 191.5 | | | | |
| | 6.8 | 202.3 | 337.7 | | | | |
| ISIS | 0.1 | 3.8 | 0.7 | 3.8 | GalNAc₃-1 | PS/20 | 822 |
| 647535 | 0.8 | 72.7 | 34.3 | | | | |
| | 2.3 | 106.8 | 111.4 | | | | |
| | 6.8 | 237.2 | 179.3 | | | | |

Metabolites of ISIS 647535 were also identified and their masses were confirmed by high resolution mass spectrometry analysis. The cleavage sites and structures of the observed metabolites are shown below. The relative 00 of full length ASO was calculated using standard procedures and the results are presented in Table 23a. The major metabolite of ISIS 647535 was full-length ASO lacking the entire conjugate (i.e. ISIS 304801), which results from cleavage at cleavage site A, shown below. Further, additional metabolites resulting from other cleavage sites were also observed. These results suggest that introducing other cleabable bonds such as esters, peptides, disulfides, phosphoramidates or acyl-hydrazones between the GalNAc₃-1 sugar and the ASO, which can be cleaved by enzymes inside the cell, or which may cleave in the reductive environment of the cytosol, or which are labile to the acidic pH inside endosomes and lyzosomes, can also be useful.

TABLE 23a
Observed full length metabolites of ISIS 647535

| | | Cleavage | Relative |
|---|---|---|---|
| Metabolite | ASO | site | % |
| 1 | ISIS 304801 | A | 36.1 |
| 2 | ISIS 304801 + dA | B | 10.5 |
| 3 | ISIS 647535 minus [3 GalNAc] | C | 16.1 |
| 4 | ISIS 647535 minus | D | 17.6 |
| | [3 GalNAc + 1 5-hydroxy-pentanoic | | |
| | acid tether] | | |
| 5 | ISIS 647535 minus | D | 9.9 |
| | [2 GalNAc + 2 5-hydroxy-pentanoic | | |
| | acid tether] | | |
| 6 | ISIS 647535 minus | D | 9.8 |
| | [3 GalNAc + 3 5-hydroxy-pentanoic | | |
| | acid tether] | | |

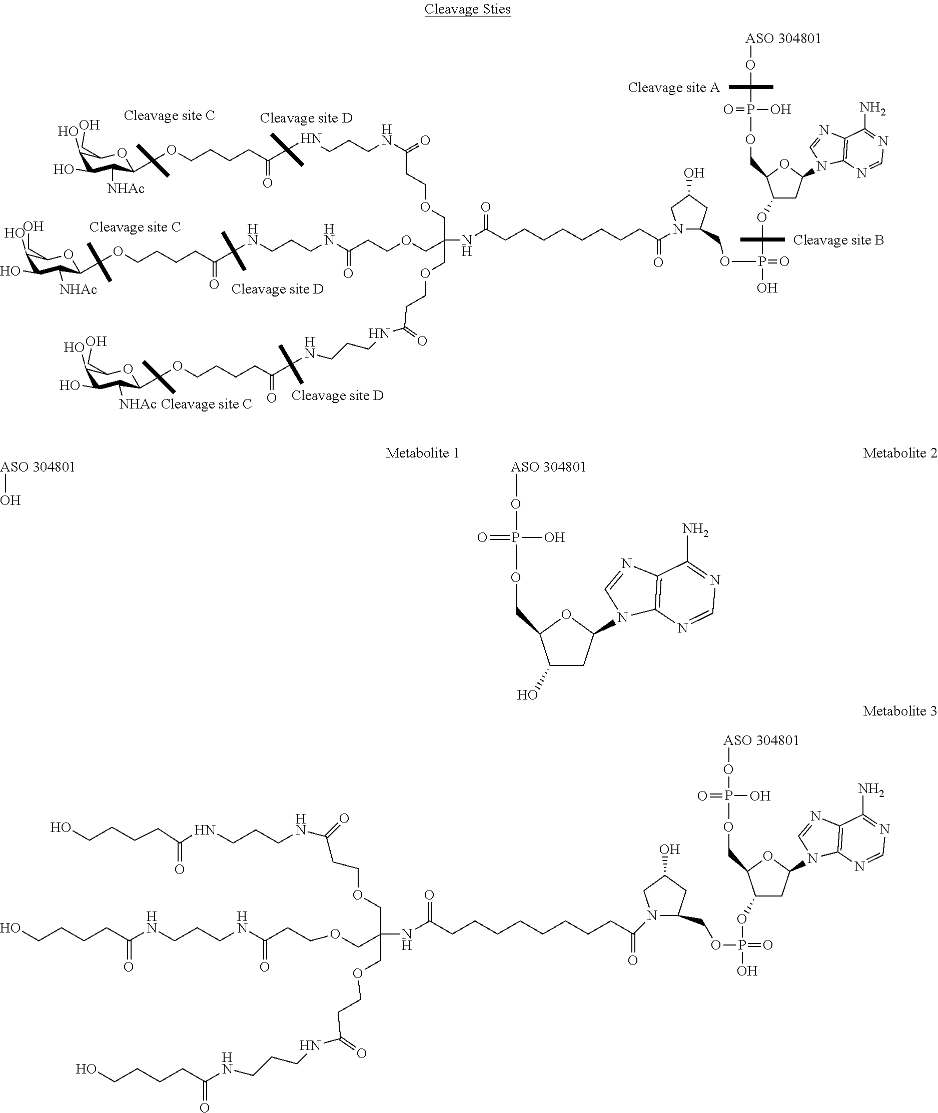

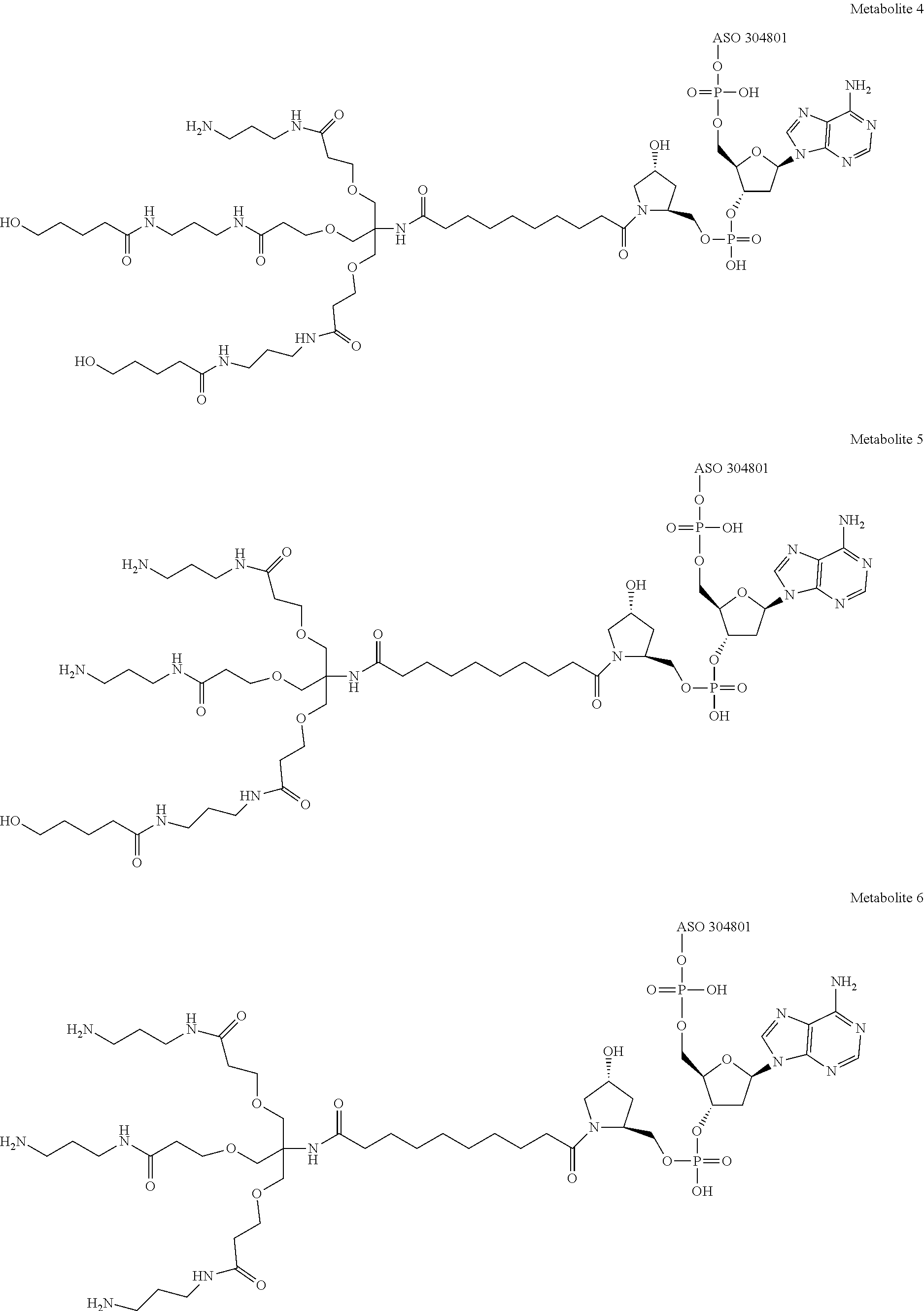

### Example 21: Antisense Inhibition of Human ApoC III in Human ApoC III Transgenic Mice in Single Administration Study

ISIS 304801, 647535 and 647536 each targeting human ApoC III and described in Table 17, were further evaluated in a single administration study for their ability to inhibit human ApoC III in human ApoC III transgenic mice.

### Treatment

Human ApoCIII transgenic mice were maintained on a 12-hour light/dark cycle and fed ad libitum Teklad lab chow. Animals were acclimated for at least 7 days in the research facility before initiation of the experiment. ASOs were prepared in PBS and sterilized by filtering through a 0.2 micron filter. ASOs were dissolved in 0.9% PBS for injection.

Human ApoC III transgenic mice were injected intraperitoneally once at the dosage shown below with ISIS 304801, 647535 or 647536 (described above) or with PBS treated control. The treatment group consisted of 3 animals and the control group consisted of 4 animals. Prior to the treatment as well as after the last dose, blood was drawn from each mouse and plasma samples were analyzed. The mice were sacrificed 72 hours following the last administration.

Samples were collected and analyzed to determine the ApoC III mRNA and protein levels in the liver; plasma triglycerides; and cholesterol, including HDL and LDL fractions were assessed as described above (Example 20). Data from those analyses are presented in Tables 24-28, below. Liver transaminase levels, alanine aminotransferase (ALT) and aspartate aminotransferase (AST), in serum were measured relative to saline injected mice using standard protocols. The ALT and AST levels showed that the antisense compounds were well tolerated at all administered doses.

These results show improvement in potency for antisense compounds comprising a GalNAc₃-1 conjugate at the 3′ terminus (ISIS 647535 and 647536) compared to the antisense compound lacking a GalNAc₃-1 conjugate (ISIS 304801). Further, ISIS 647536, which comprises a GalNAc₃-1 conjugate and some phosphodiester linkages was as potent as ISIS 647535, which comprises the same conjugate and all internucleoside linkages within the ASO are phosphorothioate.

TABLE 24
Effect of ASO treatment on ApoC III mRNA
levels in human ApoC III transgenic mice

| | Dose | % | ED₅₀ | 3′ | Internucleoside | SEQ |
|---|---|---|---|---|---|---|
| ASO | (mg/kg) | PBS | (mg/kg) | Conjugate | linkage/Length | ID No. |
| PBS | 0 | 99 | — | — | — | |
| ISIS | 1 | 104 | 13.2 | None | PS/20 | 821 |
| 304801 | 3 | 92 | | | | |
| | 10 | 71 | | | | |
| | 30 | 40 | | | | |
| ISIS | 0.3 | 98 | 1.9 | GalNAc₃-1 | PS/20 | 822 |
| 647535 | 1 | 70 | | | | |
| | 3 | 33 | | | | |
| | 10 | 20 | | | | |
| ISIS | 0.3 | 103 | 1.7 | GalNAc₃-1 | PS/PO/20 | 822 |
| 647536 | 1 | 60 | | | | |
| | 3 | 31 | | | | |
| | 10 | 21 | | | | |

TABLE 25
Effect of ASO treatment on ApoC III plasma protein
levels in human ApoC III transgenic mice

| | Dose | % | ED₅₀ | 3′ | Internucleoside | SEQ |
|---|---|---|---|---|---|---|
| ASO | (mg/kg) | PBS | (mg/kg) | Conjugate | Linkage/Length | ID No. |
| PBS | 0 | 99 | — | — | — | |
| ISIS | 1 | 104 | 23.2 | None | PS/20 | 821 |
| 304801 | 3 | 92 | | | | |
| | 10 | 71 | | | | |
| | 30 | 40 | | | | |
| ISIS | 0.3 | 98 | 2.1 | GalNAc₃-1 | PS/20 | 822 |
| 647535 | 1 | 70 | | | | |
| | 3 | 33 | | | | |
| | 10 | 20 | | | | |
| ISIS | 0.3 | 103 | 1.8 | GalNAc₃-1 | PS/PO/20 | 822 |
| 647536 | 1 | 60 | | | | |
| | 3 | 31 | | | | |
| | 10 | 21 | | | | |

TABLE 26
Effect of ASO treatment on triglyceride levels in transgenic mice

| | Dose | % | ED₅₀ | 3′ | Internucleoside | SEQ |
|---|---|---|---|---|---|---|
| ASO | (mg/kg) | PBS | (mg/kg) | Conjugate | Linkage/Length | ID No. |
| PBS | 0 | 98 | — | — | — | |
| ISIS | 1 | 80 | 29.1 | None | PS/20 | 821 |
| 304801 | 3 | 92 | | | | |
| | 10 | 70 | | | | |
| | 30 | 47 | | | | |
| ISIS | 0.3 | 100 | 2.2 | GalNAc₃-1 | PS/20 | 822 |
| 647535 | 1 | 70 | | | | |
| | 3 | 34 | | | | |
| | 10 | 23 | | | | |
| ISIS | 0.3 | 95 | 1.9 | GalNAc₃-1 | PS/PO/20 | 822 |
| 647536 | 1 | 66 | | | | |
| | 3 | 31 | | | | |
| | 10 | 23 | | | | |

TABLE 27
Effect of ASO treatment on total cholesterol
levels in transgenic mice

| | Dose | % | 3′ | Internucleoside | SEQ |
|---|---|---|---|---|---|
| ASO | (mg/kg) | PBS | Conjugate | Linkage/Length | ID No. |
| PBS | 0 | 96 | — | — | |
| ISIS | 1 | 104 | None | PS/20 | 821 |
| 304801 | 3 | 96 | | | |
| | 10 | 86 | | | |
| | 30 | 72 | | | |
| ISIS | 0.3 | 93 | GalNAc3-1 | PS/20 | 822 |
| 647535 | 1 | 85 | | | |
| | 3 | 61 | | | |
| | 10 | 53 | | | |
| ISIS | 0.3 | 115 | GalNAc3-1 | PS/PO/20 | 822 |
| 647536 | 1 | 79 | | | |
| | 3 | 51 | | | |
| | 10 | 54 | | | |

TABLE 28
Effect of ASO treatment on HDL and LDL cholesterol levels
in transgenic mice

| | Dose | HDL | LDL | 3′ | Internucleoside | SEQ |
|---|---|---|---|---|---|---|
| ASO | (mg/kg) | % PBS | % PBS | Conjugate | Linkage/Length | ID No. |
| PBS | 0 | 131 | 90 | — | — | |
| ISIS | 1 | 130 | 72 | None | PS/20 | 821 |
| 304801 | 3 | 186 | 79 | | | |
| | 10 | 226 | 63 | | | |
| | 30 | 240 | 46 | | | |
| ISIS | 0.3 | 98 | 86 | GalNAc₃-1 | PS/20 | 822 |
| 647535 | 1 | 214 | 67 | | | |
| | 3 | 212 | 39 | | | |
| | 10 | 218 | 35 | | | |
| ISIS | 0.3 | 143 | 89 | GalNAc₃-1 | PS/PO/20 | 822 |
| 647536 | 1 | 187 | 56 | | | |
| | 3 | 213 | 33 | | | |
| | 10 | 221 | 34 | | | |

These results confirm that the GalNAc₃-1 conjugate improves potency of an antisense compound. The results also show equal potency of a GalNAc₃-1 conjugated antisense compounds where the antisense oligonucleotides have mixed linkages (ISIS 647536 which has six phosphodiester linkages) and a full phosphorothioate version of the same antisense compound (ISIS 647535).

Phosphorothioate linkages provide several properties to antisense compounds. For example, they resist nuclease digestion and they bind proteins resulting in accumulation of compound in the liver, rather than in the kidney/urine. These are desirable properties, particularly when treating an indication in the liver. However, phosphorothioate linkages have also been associated with an inflammatory response. Accordingly, reducing the number of phosphorothioate linkages in a compound is expected to reduce the risk of inflammation, but also lower concentration of the compound in liver, increase concentration in the kidney and urine, decrease stability in the presence of nucleases, and lower overall potency. The present results show that a GalNAc₃-1 conjugated antisense compound where certain phosphorothioate linkages have been replaced with phosphodiester linkages is as potent against a target in the liver as a counterpart having full phosphorothioate linkages. Such compounds are expected to be less proinflammatory (See Example 24 describing an experiment showing reduction of PS results in reduced inflammatory effect).

### Example 22: Effect of GalNAc₃-1 Conjugated Modified ASO Targeting SRB-1 In Vivo

ISIS 440762 and 651900, each targeting SRB-1 and described in Table 17, were evaluated in a dose-dependent study for their ability to inhibit SRB-1 in Balb/c mice.

### Treatment

Six week old male Balb/c mice (Jackson Laboratory, Bar Harbor, ME) were injected subcutaneously once at the dosage shown below with ISIS 440762, 651900 or with PBS treated control. Each treatment group consisted of 4 animals. The mice were sacrificed 48 hours following the final administration to determine the SRB-1 mRNA levels in liver using real-time PCR and RIBOGREEN® RNA quantification reagent (Molecular Probes, Inc. Eugene, OR) according to standard protocols. SRB-1 mRNA levels were determined relative to total RNA (using Ribogreen), prior to normalization to PBS-treated control. The results below are presented as the average percent of SRB-1 mRNA levels for each treatment group, normalized to PBS-treated control and is denoted as “% PBS”.

As illustrated in Table 29, both antisense compounds lowered SRB-1 mRNA levels. Further, the antisense compound comprising the GalNAc₃-1 conjugate (ISIS 651900) was substantially more potent than the antisense compound lacking the GalNAc₃-1 conjugate (ISIS 440762). These results demonstrate that the potency benefit of GalNAc₃-1 conjugates are observed using antisense oligonucleotides complementary to a different target and having different chemically modified nucleosides, in this instance modified nucleosides comprise constrained ethyl sugar moieties (a bicyclic sugar moiety).

TABLE 29
Effect of ASO treatment on SRB-1 mRNA levels in Balb/c mice

| | Dose | Liver | ED₅₀ | 3′ | Internucleoside | SEQ |
|---|---|---|---|---|---|---|
| ASO | (mg/kg) | % PBS | (mg/kg) | Conjugate | linkage/Length | ID No. |
| PBS | 0 | 100 | | — | — | |
| ISIS | 0.7 | 85 | 2.2 | None | PS/14 | 823 |
| 440762 | 2 | 55 | | | | |
| | 7 | 12 | | | | |
| | 20 | 3 | | | | |
| ISIS | 0.07 | 98 | 0.3 | GalNAc3-1 | PS/14 | 824 |
| 651900 | 0.2 | 63 | | | | |
| | 0.7 | 20 | | | | |
| | 2 | 6 | | | | |
| | 7 | 5 | | | | |

### Example 23: Human Peripheral Blood Mononuclear Cells (hPBMC) Assay Protocol

The hPBMC assay was performed using BD Vautainer CPT tube method. A sample of whole blood from volunteered donors with informed consent at US HealthWorks clinic (Faraday & El Camino Real, Carlsbad) was obtained and collected in 4-15 BD Vacutainer CPT 8 ml tubes (VWR Cat. #BD362753). The approximate starting total whole blood volume in the CPT tubes for each donor was recorded using the PBMC assay data sheet.

The blood sample was remixed immediately prior to centrifugation by gently inverting tubes 8-10 times. CPT tubes were centrifuged at rt (18-25° C.) in a horizontal (swing-out) rotor for 30 min. at 1500-1800 RCF with brake off (2700 RPM Beckman Allegra 6R). The cells were retrieved from the buffy coat interface (between Ficoll and polymer gel layers); transferred to a sterile 50 ml conical tube and pooled up to 5 CPT tubes/50 ml conical tube/donor. The cells were then washed twice with PBS (Ca⁺⁺ Mg⁺⁺ free; GIBCO). The tubes were topped up to 50 ml and mixed by inverting several times. The sample was then centrifuged at 330×g for 15 minutes at rt (1215 RPM in Beckman Allegra 6R) and aspirated as much supernatant as possible without disturbing pellet. The cell pellet was dislodged by gently swirling tube and resuspended cells in RPMI+10% FBS+pen/strep (˜1 ml/10 ml starting whole blood volume). A 60 μl sample was pipette into a sample vial (Beckman Coulter) with 600 μl VersaLyse reagent (Beckman Coulter Cat #A09777) and was gently vortexed for 10-15 sec. The sample was allowed to incubate for 10 min. at rt and being mixed again before counting.

The cell suspension was counted on Vicell XR cell viability analyzer (Beckman Coulter) using PBMC cell type (dilution factor of 1:11 was stored with other parameters). The live cell/ml and viability were recorded. The cell suspension was diluted to 1×10⁷ live PBMC/ml in RPMI+10% FBS+pen/strep.

The cells were plated at 5×10⁵ in 50 μl/well of 96-well tissue culture plate (Falcon Microtest). 50 μl/well of 2× concentration oligos/controls diluted in RPMI+10% FBS+pen/strep. was added according to experiment template (100 μl/well total). Plates were placed on the shaker and allowed to mix for approx. 1 min. After being incubated for 24 hrs at 37° C.; 5% CO₂, the plates were centrifuged at 400×g for 10 minutes before removing the supernatant for MSD cytokine assay (i.e. human IL-6, IL-10, IL-8 and MCP-1).

### Example 24: Evaluation of Proinflammatory Effects in hPBMC Assay for GalNAc₃-1 Conjugated ASOs

The antisense oligonucleotides (ASOs) listed in Table 30 were evaluated for proinflammatory effect in hPBMC assay using the protocol described in Example 23. ISIS 353512 is an internal standard known to be a high responder for IL-6 release in the assay. The hPBMCs were isolated from fresh, volunteered donors and were treated with ASOs at 0, 0.0128, 0.064, 0.32, 1.6, 8, 40 and 200 μM concentrations. After a 24 hr treatment, the cytokine levels were measured.

The levels of IL-6 were used as the primary readout. The EC₅₀ and Eₘₐₓ was calculated using standard procedures. Results are expressed as the average ratio of Eₘₐₓ/EC₅₀ from two donors and is denoted as “Eₘₐₓ/EC₅₀.” The lower ratio indicates a relative decrease in the proinflammatory response and the higher ratio indicates a relative increase in the proinflammatory response.

With regard to the test compounds, the least proinflammatory compound was the PS/PO linked ASO (ISIS 616468). The GalNAc₃-1 conjugated ASO, ISIS 647535 was slightly less proinflammatory than its non-conjugated counterpart ISIS 304801. These results indicate that incorporation of some PO linkages reduces proinflammatory reaction and addition of a GalNAc₃-1 conjugate does not make a compound more proinflammatory and may reduce proinflammatory response. Accordingly, one would expect that an antisense compound comprising both mixed PS/PO linkages and a GalNAc₃-1 conjugate would produce lower proinflammatory responses relative to full PS linked antisense compound with or without a GalNAc₃-1 conjugate. These results show that GalNAc₃0.1 conjugated antisense compounds, particularly those having reduced PS content are less proinflammatory.

Together, these results suggest that a GalNAc₃-1 conjugated compound, particularly one with reduced PS content, can be administered at a higher dose than a counterpart full PS antisense compound lacking a GalNAc₃-1 conjugate. Since half-life is not expected to be substantially different for these compounds, such higher administration would result in less frequent dosing. Indeed such administration could be even less frequent, because the GalNAc₃-1 conjugated compounds are more potent (See Examples 20-22) and re-dosing is necessary once the concentration of a compound has dropped below a desired level, where such desired level is based on potency.

TABLE 30
Modified ASOs

| | | | SEQ ID |
|---|---|---|---|
| ASO | Sequence (5′ to 3′) | Target | No. |
| ISIS | Gₑₛ^{m}CₑₛTₑₛGₑₛAₑₛT_{ds}T_{ds}A_{ds}G_{ds}A_{ds}G_{ds} | TNFα | 825 |
| 104838 | A_{ds}G_{ds}A_{ds}G_{ds}GₑₛTₑₛ^{m}Cₑₛ^{m}Cₑₛ^{m}Cₑ | | |
| ISIS | Tₑₛ^{m}Cₑₛ^{m}Cₑₛ^{m}C_{ds}A_{ds}T_{ds}T_{ds}T_{ds}^{m}C_{ds}A_{ds}G_{ds} | CRP | 826 |
| 353512 | G_{ds}A_{ds}G_{ds}A_{ds}^{m}C_{ds}^{m}C_{ds}TₑₛGₑₛGₑ | | |
| ISIS | AₑₛGₑₛ^{m}CₑₛTₑₛTₑₛ^{m}C_{ds}T_{ds}T_{ds}G_{ds}T_{ds} | ApoC | 821 |
| 304801 | ^{m}C_{ds}^{m}C_{ds}A_{ds}G_{ds}^{m}C_{ds}TₑₛTₑₛTₑₛAₑₛTₑ | III | |
| ISIS | AₑₛGₑₛ^{m}CₑₛTₑₛTₑₛ^{m}C_{ds}T_{ds}T_{ds}G_{ds}T_{ds} | | |
| 647535 | ^{m}C_{ds}^{m}C_{ds}A_{ds}G_{ds}^{m}C_{ds}TₑₛTₑₛTₑₛAₑₛTₑₒ- | ApoC | 822 |
| | A_{do′}-GalNAc₃-1ₐ | III | |
| ISIS | AₑₛGₑₒ^{m}CₑₒTₑₒTₑₒ^{m}C_{ds}T_{ds}T_{ds}G_{ds}T_{ds} | ApoC | 821 |
| 616468 | ^{m}C_{ds}^{m}C_{ds}A_{ds}G_{ds}^{m}C_{ds}TₑₒTₑₒTₑₛAₑₛTₑ | III | |

Subscripts: “e” indicates 2′-MOE modified nucleoside; “d” indicates β-D-2′-deoxyribonucleoside; “k” indicates 6′-(S)—CH3 bicyclic nucleoside (e.g. cEt); “s” indicates phosphorothioate internucleoside linkages (PS); “o” indicates phosphodiester internucleoside linkages (PO); and “o′” indicates —O—P(═O)(OH)—. Superscript “m” indicates 5-methylcytosines. “A_{do′}-GalNAc₃-1ₐ” indicates a conjugate having the structure GalNAc₃-1 shown in Example 9 attached to the 3′-end of the antisense oligonucleotide, as indicated.

TABLE 31
Proinflammatory Effect of ASOs targeting ApoC III in hPBMC assay

| | EC₅₀ | Eₘₐₓ | | | Internucleoside | SEQ |
|---|---|---|---|---|---|---|
| ASO | (μM) | (μM) | Eₘₐₓ/EC₅₀ | 3′ Conjugate | Linkage/Length | ID No. |
| ISIS 353512 | 0.01 | 265.9 | 26,590 | None | PS/20 | 826 |
| (high responder) | | | | | | |
| ISIS 304801 | 0.07 | 106.55 | 1,522 | None | PS/20 | 821 |
| ISIS 647535 | 0.12 | 138 | 1,150 | GalNAc3-1 | PS/20 | 822 |
| ISIS 616468 | 0.32 | 71.52 | 224 | None | PS/PO/20 | 821 |

### Example 25: Effect of GalNAc₃-1 Conjugated Modified ASO Targeting Human ApoC III In Vitro

ISIS 304801 and 647535 described above were tested in vitro. Primary hepatocyte cells from transgenic mice at a density of 25,000 cells per well were treated with 0.03, 0.08, 0.24, 0.74, 2.22, 6.67 and 20 μM concentrations of modified oligonucleotides. After a treatment period of approximately 16 hours, RNA was isolated from the cells and mRNA levels were measured by quantitative real-time PCR and the hApoC III mRNA levels were adjusted according to total RNA content, as measured by RIBOGREEN.

The IC₅₀ was calculated using the standard methods and the results are presented in Table 32. As illustrated, comparable potency was observed in cells treated with ISIS 647535 as compared to the control, ISIS 304801.

TABLE 32
Modified ASO targeting human ApoC III in primary hepatocytes

| | IC₅₀ | | Internucleoside | SEQ |
|---|---|---|---|---|
| ASO | (μM) | 3′ Conjugate | linkage/Length | ID No. |
| ISIS | 0.44 | None | PS/20 | 821 |
| 304801 | | | | |
| ISIS | 0.31 | GalNAc3-1 | PS/20 | 822 |
| 647535 | | | | |

In this experiment, the large potency benefits of GalNAc₃-1 conjugation that are observed in vivo were not observed in vitro. Subsequent free uptake experiments in primary hepatocytes in vitro did show increased potency of oligonucleotides comprising various GalNAc conjugates relative to oligonucleotides that lacking the GalNAc conjugate. (see Examples 60, 82, and 92)

### Example 26: Effect of PO/PS Linkages on ApoC III ASO Activity

Human ApoC III transgenic mice were injected intraperitoneally once at 25 mg/kg of ISIS 304801, or ISIS 616468 (both described above) or with PBS treated control once per week for two weeks. The treatment group consisted of 3 animals and the control group consisted of 4 animals. Prior to the treatment as well as after the last dose, blood was drawn from each mouse and plasma samples were analyzed. The mice were sacrificed 72 hours following the last administration.

Samples were collected and analyzed to determine the ApoC III protein levels in the liver as described above (Example 20). Data from those analyses are presented in Table 33, below.

These results show reduction in potency for antisense compounds with PO/PS (ISIS 616468) in the wings relative to full PS (ISIS 304801).

TABLE 33
Effect of ASO treatment on ApoC III protein
levels in human ApoC III transgenic mice

| | Dose | % | 3′ | Internucleoside | SEQ |
|---|---|---|---|---|---|
| ASO | (mg/kg) | PBS | Conjugate | linkage/Length | ID No. |
| PBS | 0 | 99 | — | — | |
| ISIS | 25 | 24 | None | Full PS | 821 |
| 304801 | mg/kg/wk | | | | |
| | for 2 wks | | | | |
| ISIS | 25 | 40 | None | 14 PS/6 PO | 821 |
| 616468 | mg/kg/wk | | | | |
| | for 2 wks | | | | |

### Example 27: Compound 56

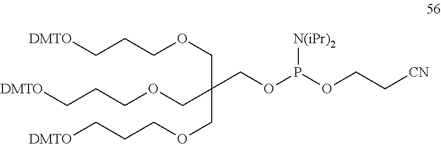

Compound 56 is commercially available from Glen Research or may be prepared according to published procedures reported by Shchepinov et al., Nucleic Acids Research, 1997, 25(22), 4447-4454.

### Example 28: Preparation of Compound 60

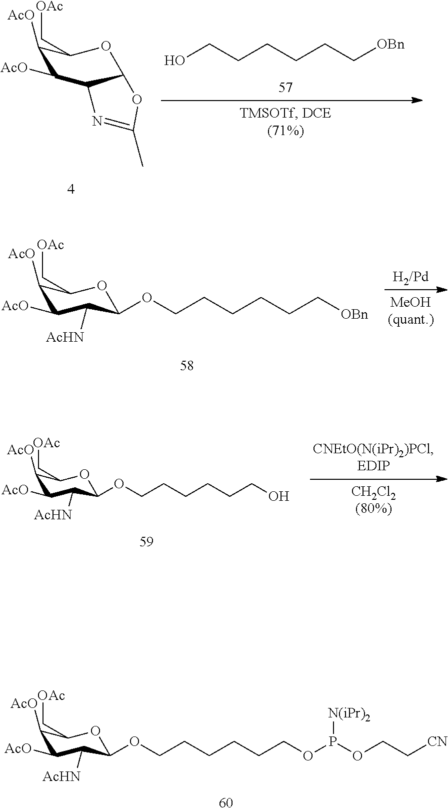

Compound 4 was prepared as per the procedures illustrated in Example 2. Compound 57 is commercially available. Compound 60 was confirmed by structural analysis.

Compound 57 is meant to be representative and not intended to be limiting as other mono-protected substituted or unsubstituted alkyl diols including but not limited to those presented in the specification herein can be used to prepare phosphoramidites having a predetermined composition.

### Example 29: Preparation of Compound 63

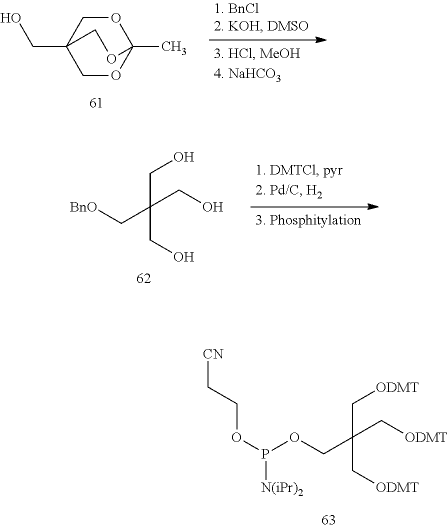

Compounds 61 and 62 are prepared using procedures similar to those reported by Tober et al., Eur. J. Org. Chem., 2013, 3, 566-577; and Jiang et al., Tetrahedron, 2007, 63(19), 3982-3988.

Alternatively, Compound 63 is prepared using procedures similar to those reported in scientific and patent literature by Kim et al., Synlett, 2003, 12, 1838-1840; and Kim et al., published PCT International Application, WO 2004063208.Example 30: Preparation of Compound 63b

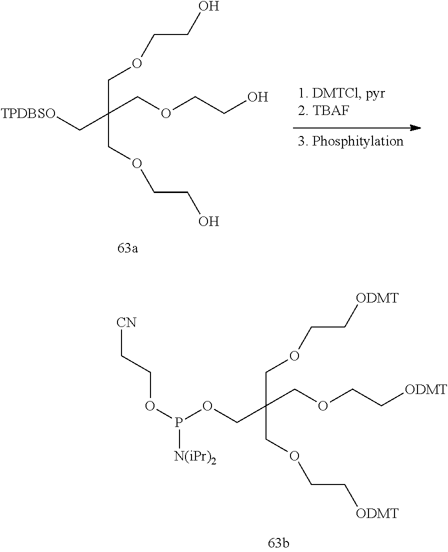

Compound 63a is prepared using procedures similar to those reported by Hanessian et al., Canadian Journal of Chemistry, 1996, 74(9), 1731-1737.

### Example 31: Preparation of Compound 63d

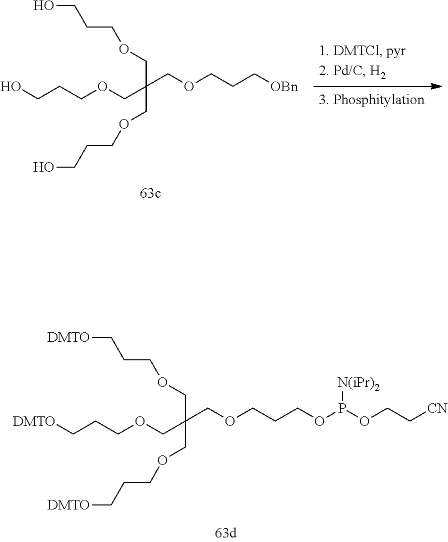

Compound 63c is prepared using procedures similar to those reported by Chen et al., Chinese Chemical Letters, 1998, 9(5), 451-453.

### Example 32: Preparation of Compound 67

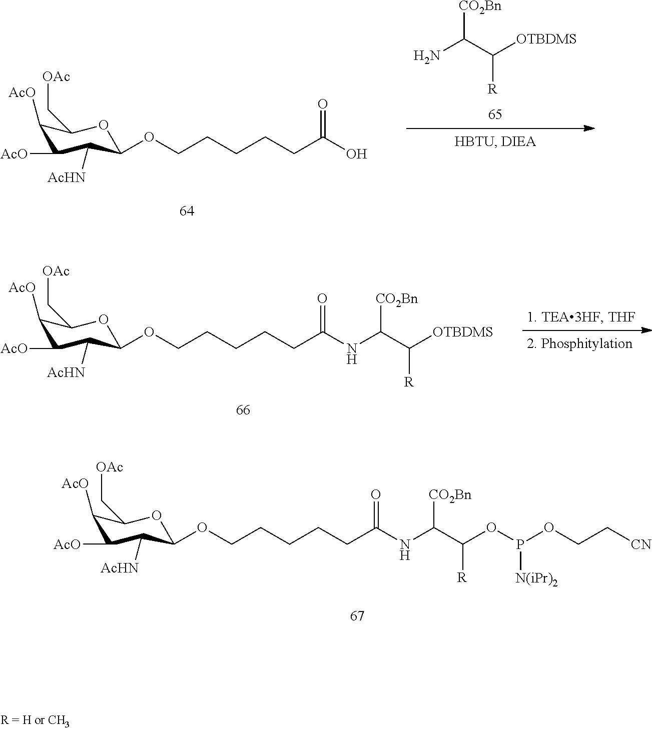

Compound 64 was prepared as per the procedures illustrated in Example 2. Compound 65 is prepared using procedures similar to those reported by Or et al., published PCT International Application, WO 2009003009. The protecting groups used for Compound 65 are meant to be representative and not intended to be limiting as other protecting groups including but not limited to those presented in the specification herein can be used.

### Example 33: Preparation of Compound 70

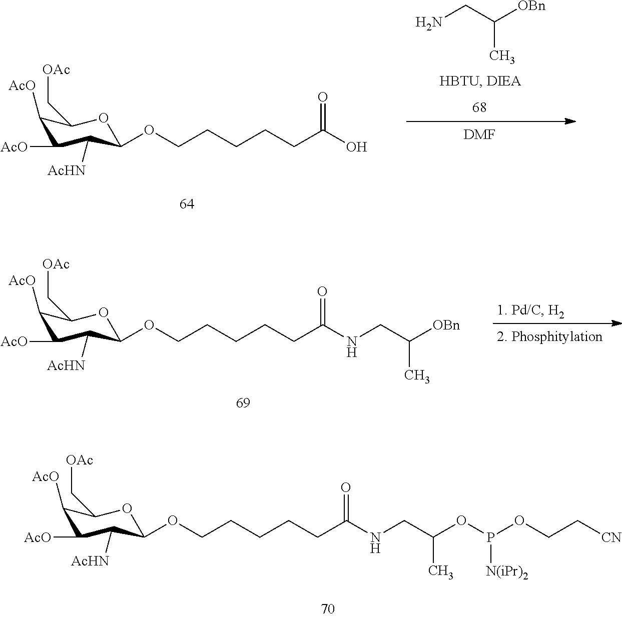

Compound 64 was prepared as per the procedures illustrated in Example 2. Compound 68 is commercially available. The protecting group used for Compound 68 is meant to be representative and not intended to be limiting as other protecting groups including but not limited to those presented in the specification herein can be used.

### Example 34: Preparation of Compound 75a

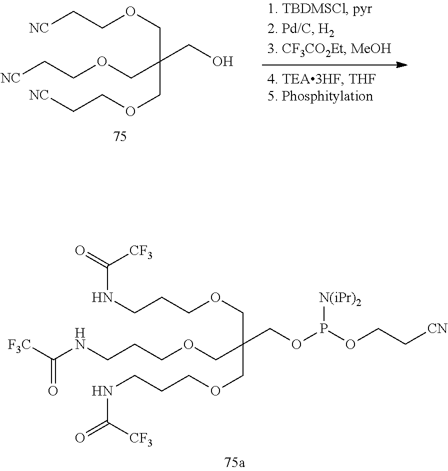

Compound 75 is prepared according to published procedures reported by Shchepinov et al., Nucleic Acids Research, 1997, 25(22), 4447-4454.

### Example 35: Preparation of Compound 79

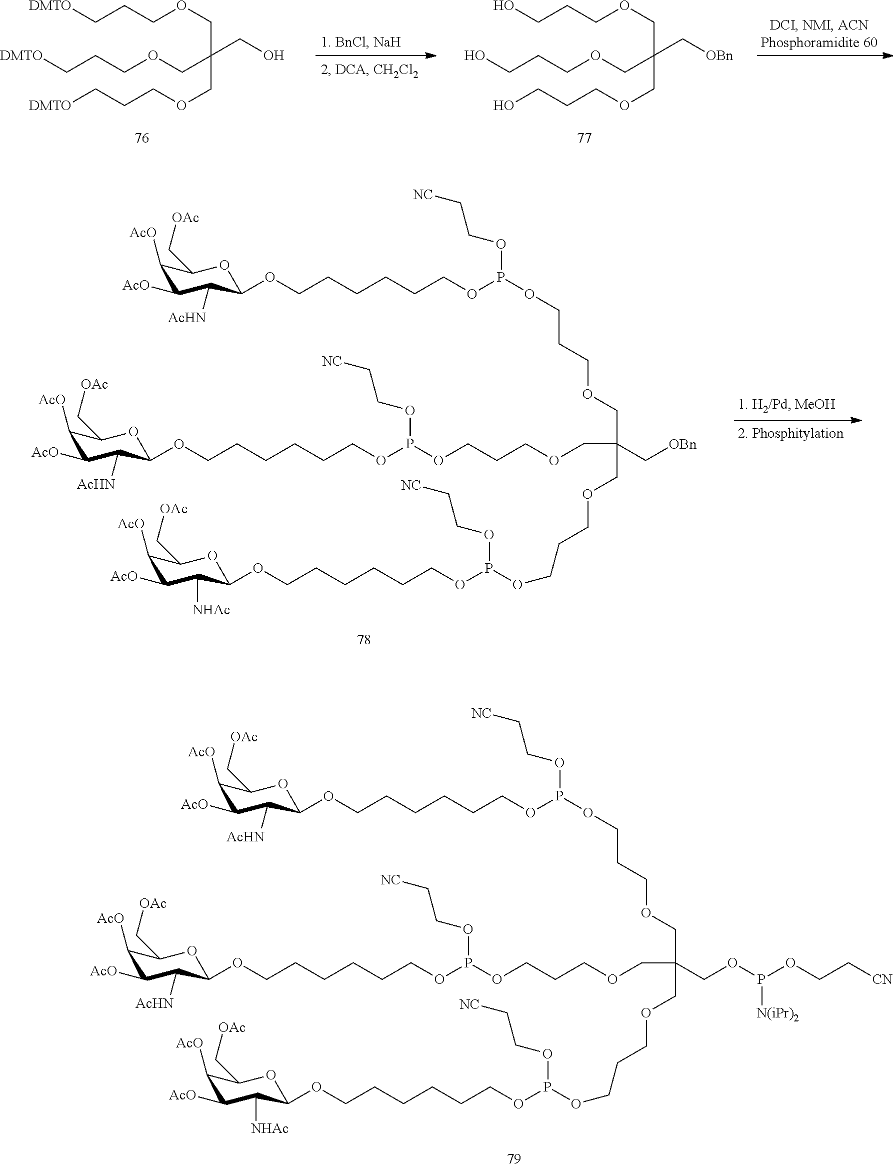

Compound 76 was prepared according to published procedures reported by Shchepinov et al., Nucleic Acids Research, 1997, 25(22), 4447-4454.

### Example 36: Preparation of Compound 79a

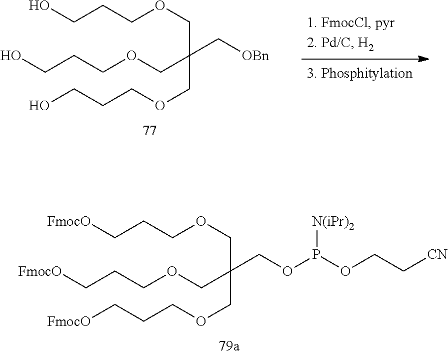

Compound 77 is prepared as per the procedures illustrated in Example 35.

### Example 37: General Method for the Preparation of Conjugated Oligomeric Compound 82 Comprising a Phosphodiester Linked GalNAc₃-2 Conjugate at 5′ Terminus Via Solid Support (Method I)

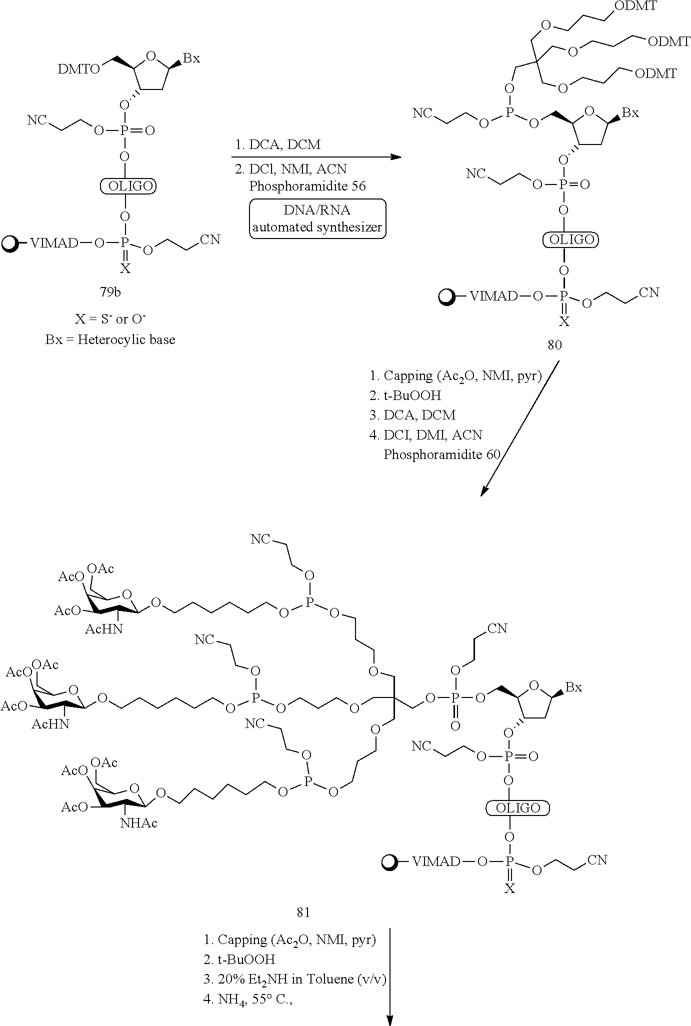

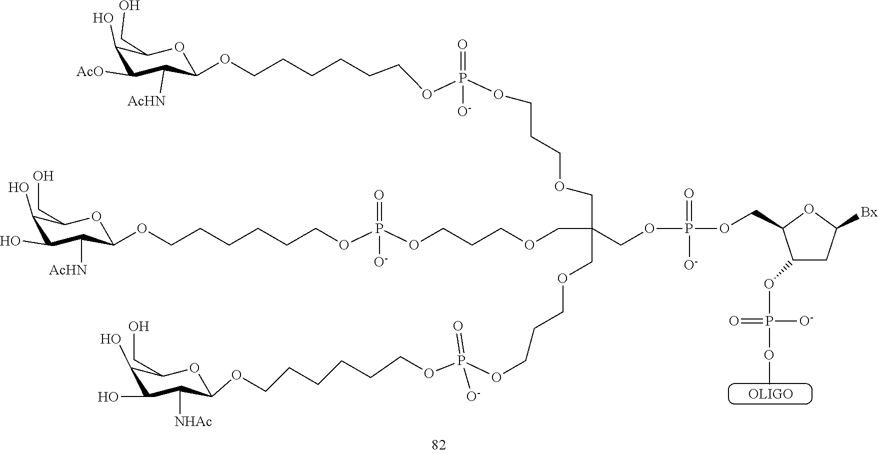

wherein GalNAc₃-2 has the structure:

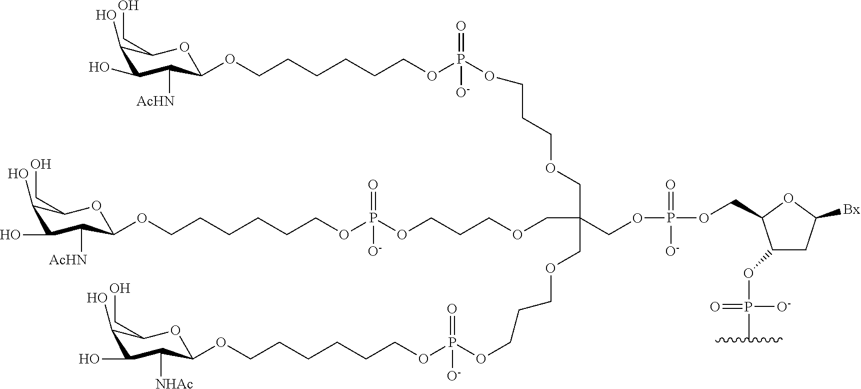

The GalNAc3 cluster portion of the conjugate group GalNAc₃-2 (GalNAc₃-2ₐ) can be combined with any cleavable moiety to provide a variety of conjugate groups. Wherein GalNAc₃-2ₐ has the formula:

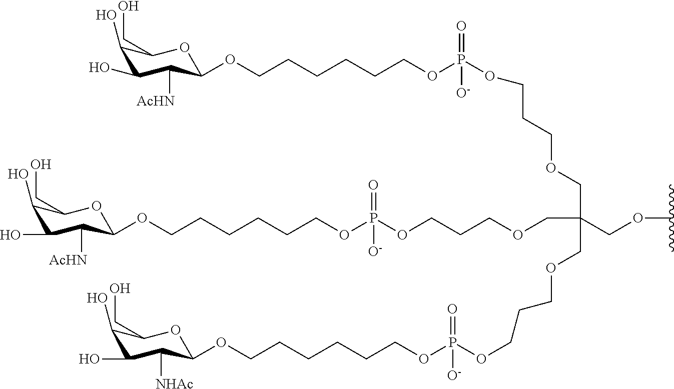

The VIMAD-bound oligomeric compound 79b was prepared using standard procedures for automated DNA/RNA synthesis (see Dupouy et al., Angew. Chem. Int. Ed., 2006, 45, 3623-3627). The phosphoramidite Compounds 56 and 60 were prepared as per the procedures illustrated in Examples 27 and 28, respectively. The phosphoramidites illustrated are meant to be representative and not intended to be limiting as other phosphoramidite building blocks including but not limited those presented in the specification herein can be used to prepare an oligomeric compound having a phosphodiester linked conjugate group at the 5′ terminus. The order and quantity of phosphoramidites added to the solid support can be adjusted to prepare the oligomeric compounds as described herein having any predetermined sequence and composition.

### Example 38: Alternative Method for the Preparation of Oligomeric Compound 82 Comprising a Phosphodiester Linked GalNAc₃-2 Conjugate at 5′ Terminus (Method II)

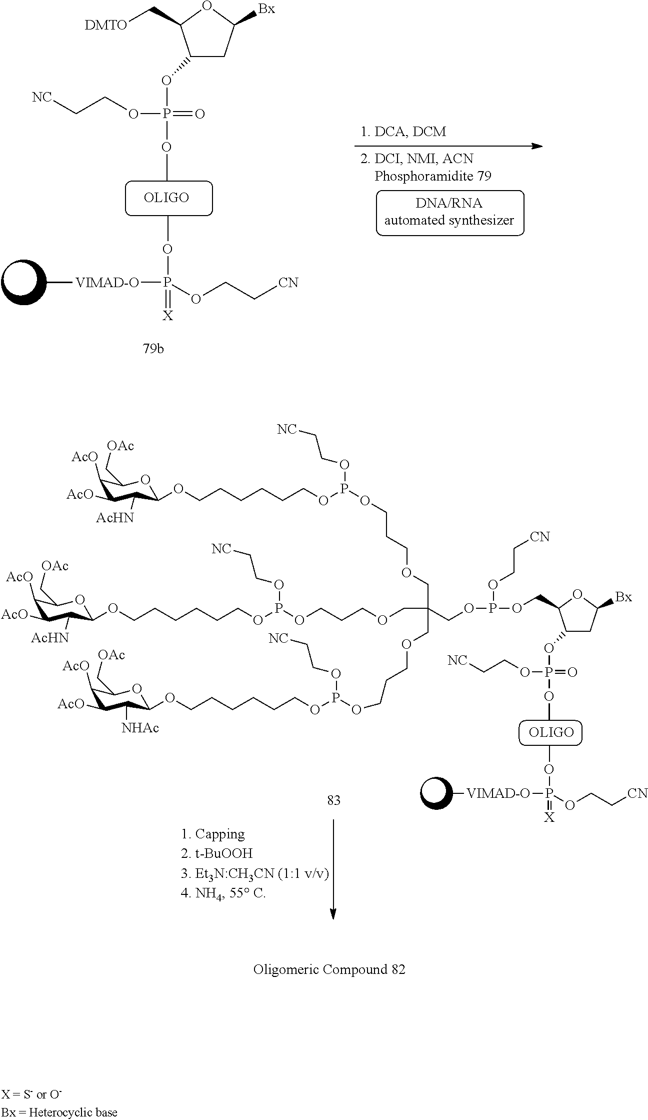

The VIMAD-bound oligomeric compound 79b was prepared using standard procedures for automated DNA/RNA synthesis (see Dupouy et al., Angew. Chem. Int. Ed., 2006, 45, 3623-3627). The GalNAc₃-2 cluster phosphoramidite, Compound 79 was prepared as per the procedures illustrated in Example 35. This alternative method allows a one-step installation of the phosphodiester linked GalNAc₃-2 conjugate to the oligomeric compound at the final step of the synthesis. The phosphoramidites illustrated are meant to be representative and not intended to be limiting, as other phosphoramidite building blocks including but not limited to those presented in the specification herein can be used to prepare oligomeric compounds having a phosphodiester conjugate at the 5′ terminus. The order and quantity of phosphoramidites added to the solid support can be adjusted to prepare the oligomeric compounds as described herein having any predetermined sequence and composition.

### Example 39: General Method for the Preparation of Oligomeric Compound 83h Comprising a GalNAc₃-3 Conjugate at the 5′ Terminus (GalNAc₃-1 Modified for 5′ End Attachment) Via Solid Support

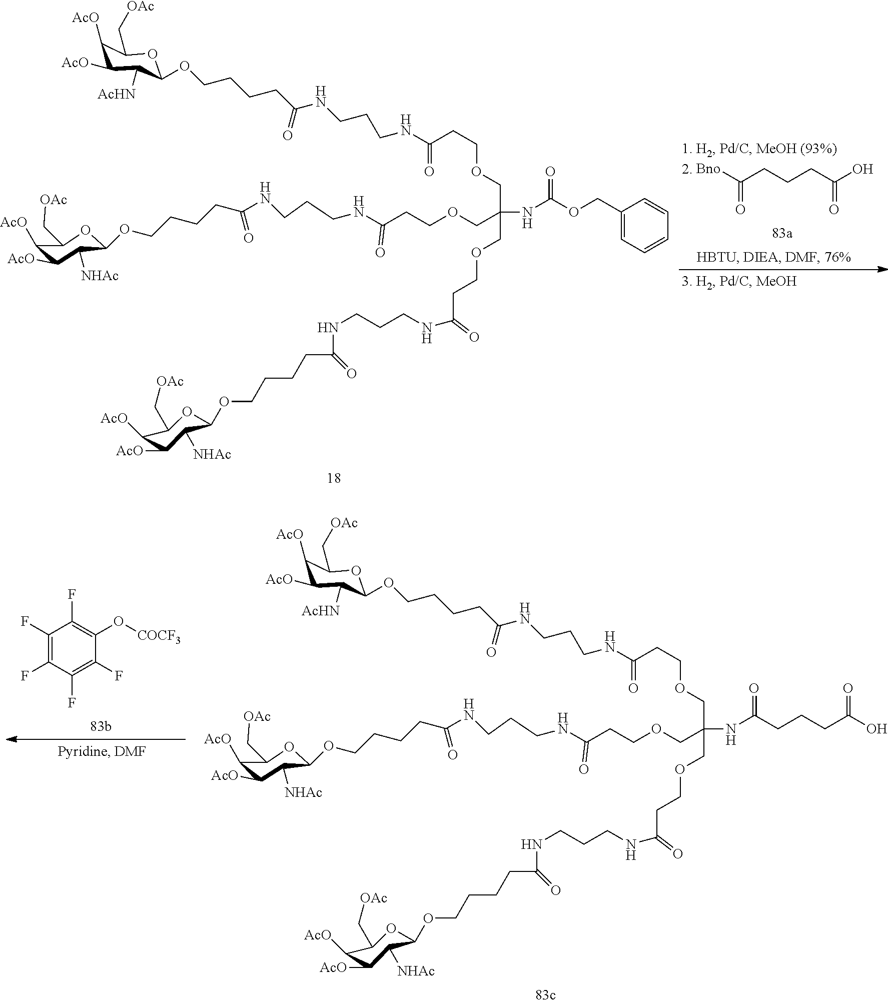

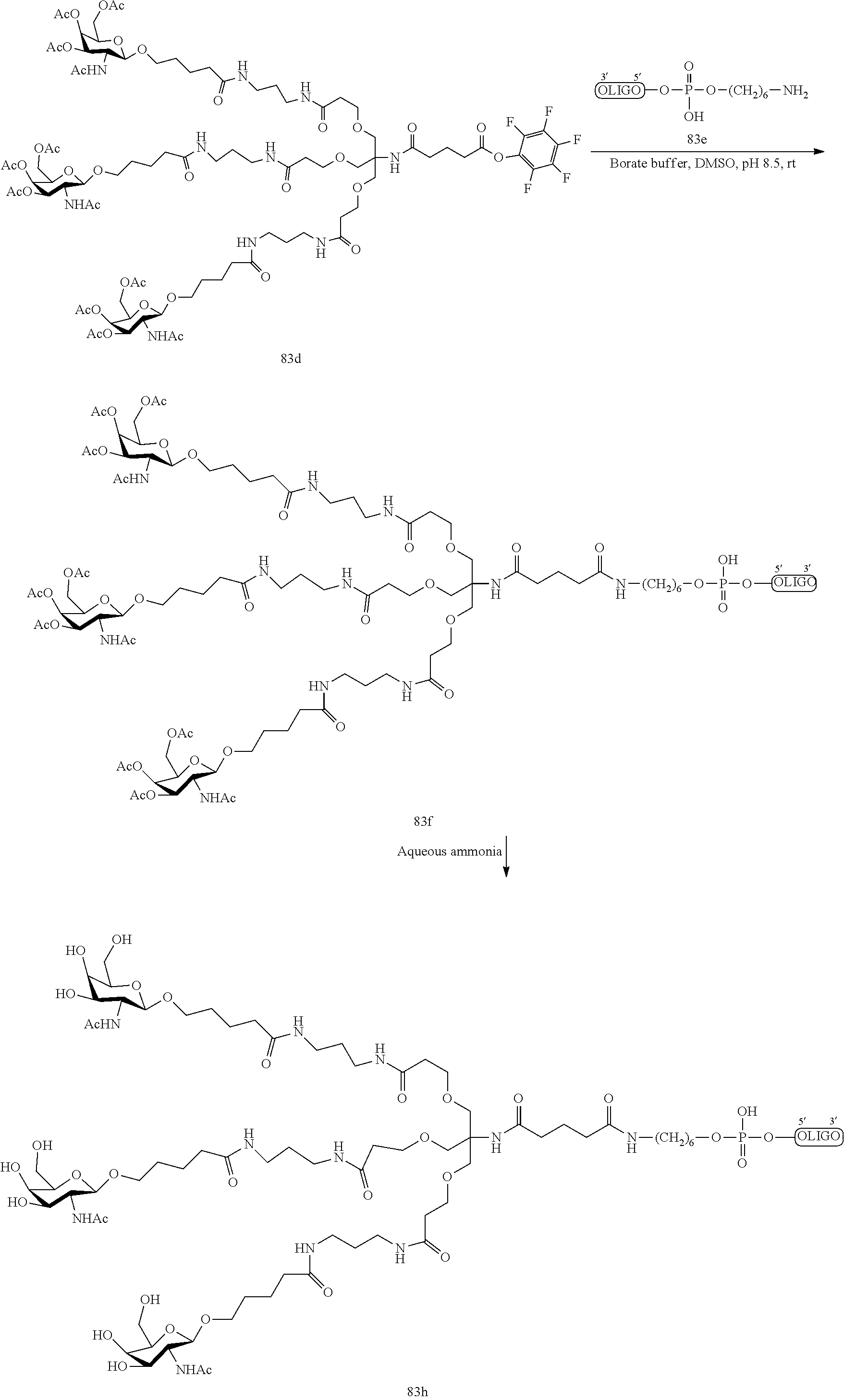

Compound 18 was prepared as per the procedures illustrated in Example 4. Compounds 83a and 83b are commercially available. Oligomeric Compound 83e comprising a phosphodiester linked hexylamine was prepared using standard oligonucleotide synthesis procedures. Treatment of the protected oligomeric compound with aqueous ammonia provided the 5′-GalNAc₃-3 conjugated oligomeric compound (83h).

Wherein GalNAc₃-3 has the structure:

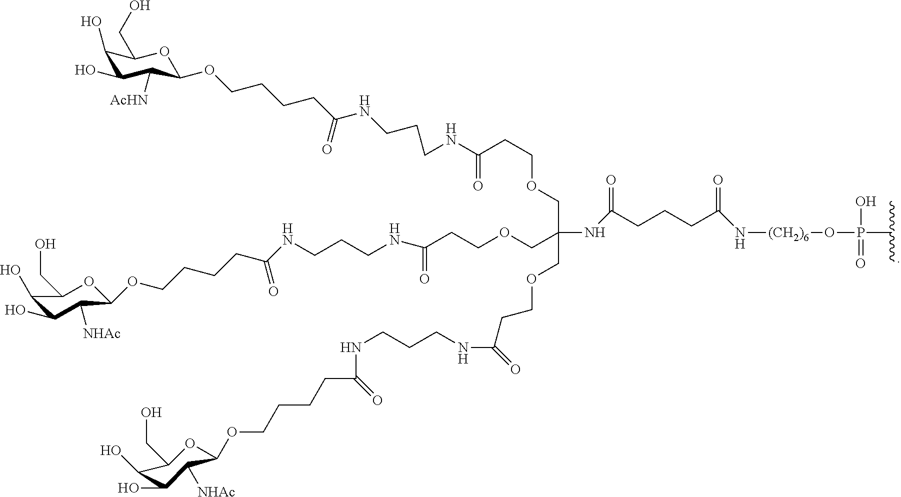

The GalNAc3 cluster portion of the conjugate group GalNAc₃-3 (GalNAc₃-3ₐ) can be combined with any cleavable moiety to provide a variety of conjugate groups. Wherein GalNAc₃-3ₐ has the formula:

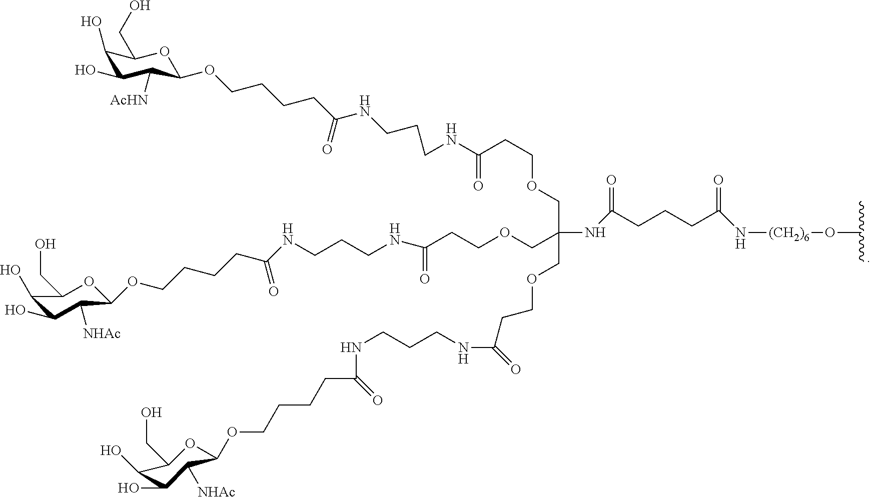

### Example 40: General Method for the Preparation of Oligomeric Compound 89 Comprising a Phosphodiester Linked GalNAc₃-4 Conjugate at the 3′ Terminus Via Solid Support

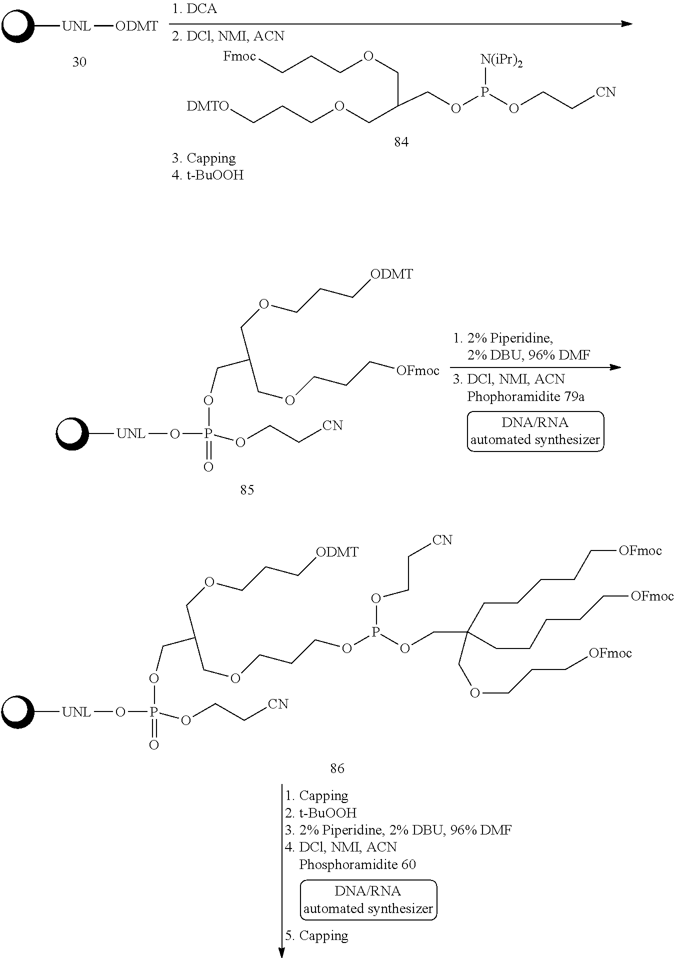

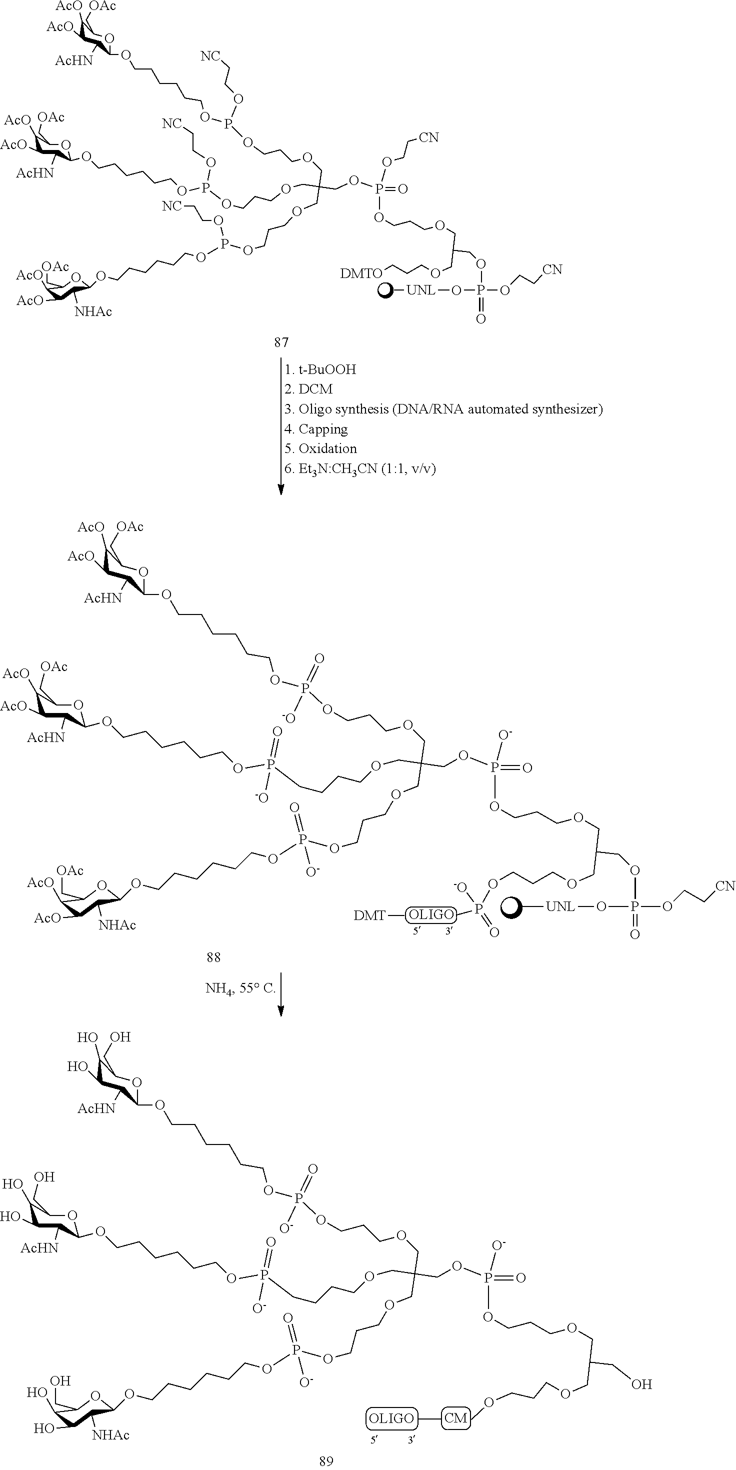

Wherein GalNAc₃-4 has the structure:

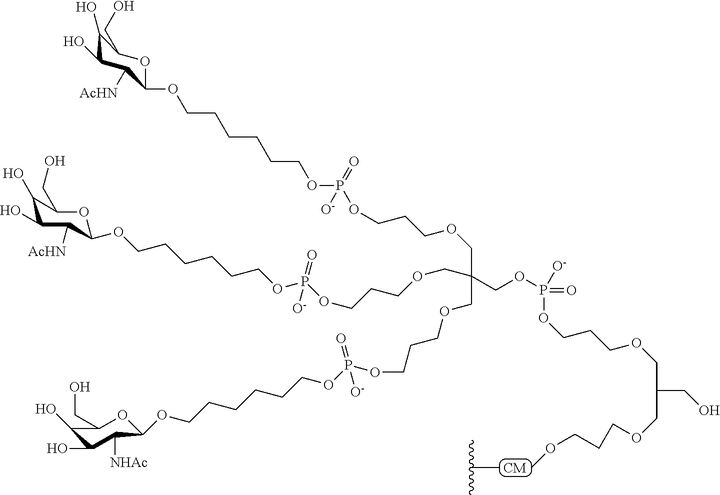

Wherein CM is a cleavable moiety. In certain embodiments, cleavable moiety is:

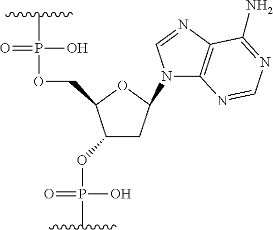 [SMILES: *P(=O)(O)OC[C@H]1O[C@@H](n2cnc3c(N)ncnc32)C[C@@H]1OP(*)(=O)O]

The GalNAc3 cluster portion of the conjugate group GalNAc₃-4 (GalNAc₃-4a) can be combined with any cleavable moiety to provide a variety of conjugate groups. Wherein GalNAc₃-4a has the formula:

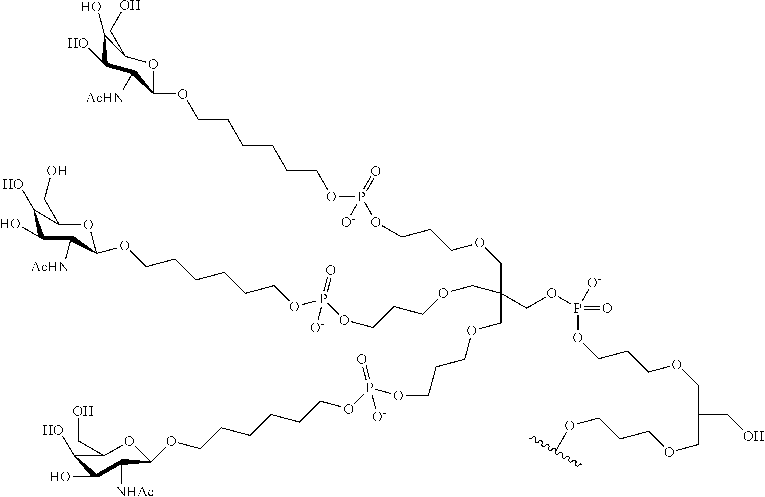

The protected Unylinker functionalized solid support Compound 30 is commercially available. Compound 84 is prepared using procedures similar to those reported in the literature (see Shchepinov et al., Nucleic Acids Research, 1997, 25(22), 4447-4454; Shchepinov et al., Nucleic Acids Research, 1999, 27, 3035-3041; and Hornet et al., Nucleic Acids Research, 1997, 25, 4842-4849).

The phosphoramidite building blocks, Compounds 60 and 79a are prepared as per the procedures illustrated in Examples 28 and 36. The phosphoramidites illustrated are meant to be representative and not intended to be limiting as other phosphoramidite building blocks can be used to prepare an oligomeric compound having a phosphodiester linked conjugate at the 3′ terminus with a predetermined sequence and composition. The order and quantity of phosphoramidites added to the solid support can be adjusted to prepare the oligomeric compounds as described herein having any predetermined sequence and composition.

### Example 41: General Method for the Preparation of ASOs Comprising a Phosphodiester Linked GalNAc₃-2 (See Example 37, Bx is Adenine) Conjugate at the 5′ Position Via Solid Phase Techniques (Preparation of ISIS 661134)

Unless otherwise stated, all reagents and solutions used for the synthesis of oligomeric compounds are purchased from commercial sources. Standard phosphoramidite building blocks and solid support are used for incorporation nucleoside residues which include for example T, A, G, and ^{m}C residues. Phosphoramidite compounds 56 and 60 were used to synthesize the phosphodiester linked GalNAc₃-2 conjugate at the 5′ terminus. A 0.1 M solution of phosphoramidite in anhydrous acetonitrile was used for β-D-2′-deoxyribonucleoside and 2′-MOE.

The ASO syntheses were performed on ABI 394 synthesizer (1-2 μmol scale) or on GE Healthcare Bioscience AKTA oligopilot synthesizer (40-200 μmol scale) by the phosphoramidite coupling method on VIMAD solid support (110 μmol/g, Guzaev et al., 2003) packed in the column. For the coupling step, the phosphoramidites were delivered at a 4 fold excess over the initial loading of the solid support and phosphoramidite coupling was carried out for 10 min. All other steps followed standard protocols supplied by the manufacturer. A solution of 6% dichloroacetic acid in toluene was used for removing the dimethoxytrityl (DMT) groups from 5′-hydroxyl groups of the nucleotide. 4,5-Dicyanoimidazole (0.7 M) in anhydrous CH3CN was used as activator during the coupling step. Phosphorothioate linkages were introduced by sulfurization with 0.1 M solution of xanthane hydride in 1:1 pyridine/CH3CN for a contact time of 3 minutes. A solution of 20% tert-butylhydroperoxide in CH3CN containing 6% water was used as an oxidizing agent to provide phosphodiester internucleoside linkages with a contact time of 12 minutes.

After the desired sequence was assembled, the cyanoethyl phosphate protecting groups were deprotected using a 20% diethylamine in toluene (v/v) with a contact time of 45 minutes. The solid-support bound ASOs were suspended in aqueous ammonia (28-30 wt %) and heated at 55° C. for 6 h. The unbound ASOs were then filtered and the ammonia was boiled off. The residue was purified by high pressure liquid chromatography on a strong anion exchange column (GE Healthcare Bioscience, Source 30Q, 30 μm, 2.54×8 cm, A=100 mM ammonium acetate in 30% aqueous CH3CN, B=1.5 M NaBr in A, 0-40% of B in 60 min, flow 14 mL min-1, λ=260 nm). The residue was desalted by HPLC on a reverse phase column to yield the desired ASOs in an isolated yield of 15-30% based on the initial loading on the solid support. The ASOs were characterized by ion-pair-HPLC coupled MS analysis with Agilent 1100 MSD system.

TABLE 34
ASO comprising a phosphodiester linked GalNAc₃-2
conjugate at the 5′ position targeting SRB-1

| ISIS | | CalCd | Observed | SEQ ID |
|---|---|---|---|---|
| No. | Sequence (5′ to 3′) | Mass | Mass | No. |
| 661134 | GalNAc₃-2ₐ-_{o′} | 6482.2 | 6481.6 | 827 |
| | A_{do}Tₖₛ^{m}CₖₛA_{ds}G_{ds}T_{ds}^{m}C_{ds}A_{ds}T_{ds}G_{ds} | | | |
| | A_{ds}^{m}C_{ds}T_{ds}Tₖₛ^{m}Cₖ | | | |

Subscripts: “e” indicates 2′-MOE modified nucleoside; “d” indicates 3-D-2′-deoxyribonucleoside; “k” indicates 6′-(S)—CH3 bicyclic nucleoside (e.g. cEt); “s” indicates phosphorothioate internucleoside linkages (PS); “o” indicates phosphodiester internucleoside linkages (PO); and “o′” indicates —O—P(═O)(OH)—. Superscript “m” indicates 5-methylcytosines. The structure of GalNAc₃-2ₐ is shown in Example 37.

### Example 42: General Method for the Preparation of ASOs Comprising a GalNAc₃-3 Conjugate at the 5′ Position Via Solid Phase Techniques (Preparation of ISIS 661166)

The synthesis for ISIS 661166 was performed using similar procedures as illustrated in Examples 39 and 41.

ISIS 661166 is a 5-10-5 MOE gapmer, wherein the 5′ position comprises a GalNAc₃-3 conjugate. The ASO was characterized by ion-pair-HPLC coupled MS analysis with Agilent 1100 MSD system.

TABLE 34a
ASO comprising a GalNAc₃-3 conjugate at the 5′ position via
a hexylamino phosphodiester linkage targeting Malat-1

| ISIS | | | Calcd | Observed | SEQ ID |
|---|---|---|---|---|---|
| No. | Sequence (5′ to 3′) | Conjugate | Mass | Mass | No. |
| 661166 | 5′-GalNAc₃-3_{a-o′}^{m}CₑₛGₑₛGₑₛTₑₛGₑₛ | 5′-GalNAc₃-3 | 8992.16 | 8990.51 | 828 |
| | ^{m}C_{ds}A_{ds}A_{ds}G_{ds}G_{ds}^{m}C_{ds}T_{ds}T_{ds}A_{ds}G_{ds} | | | | |
| | GₑₛAₑₛAₑₛTₑₛTₑ | | | | |

Subscripts: “e” indicates 2′-MOE modified nucleoside; “d” indicates 3-D-2′-deoxyribonucleoside; “s” indicates phosphorothioate internucleoside linkages (PS); “o” indicates phosphodiester internucleoside linkages (PO); and “o′” indicates —O—P(═O)(OH)—. Superscript “m” indicates 5-methylcytosines. The structure of “5′-GalNAc₃-3ₐ” is shown in Example 39.

### Example 43: Dose-Dependent Study of Phosphodiester Linked GalNAc₃-2 (See Examples 37 and 41, Bx is Adenine) at the 5′ Terminus Targeting SRB-1 In Vivo

ISIS 661134 (see Example 41) comprising a phosphodiester linked GalNAc₃-2 conjugate at the 5′ terminus was tested in a dose-dependent study for antisense inhibition of SRB-1 in mice. Unconjugated ISIS 440762 and 651900 (GalNAc₃-1 conjugate at 3′ terminus, see Example 9) were included in the study for comparison and are described previously in Table 17.

### Treatment

Six week old male Balb/c mice (Jackson Laboratory, Bar Harbor, ME) were injected subcutaneously once at the dosage shown below with ISIS 440762, 651900, 661134 or with PBS treated control. Each treatment group consisted of 4 animals. The mice were sacrificed 72 hours following the final administration to determine the liver SRB-1 mRNA levels using real-time PCR and RIBOGREEN® RNA quantification reagent (Molecular Probes, Inc. Eugene, OR) according to standard protocols. SRB-1 mRNA levels were determined relative to total RNA (using Ribogreen), prior to normalization to PBS-treated control. The results below are presented as the average percent of SRB-1 mRNA levels for each treatment group, normalized to PBS-treated control and is denoted as “% PBS”. The ED_{50S} were measured using similar methods as described previously and are presented below.

As illustrated in Table 35, treatment with antisense oligonucleotides lowered SRB-1 mRNA levels in a dose-dependent manner. Indeed, the antisense oligonucleotides comprising the phosphodiester linked GalNAc₃-2 conjugate at the 5′ terminus (ISIS 661134) or the GalNAc₃-1 conjugate linked at the 3′ terminus (ISIS 651900) showed substantial improvement in potency compared to the unconjugated antisense oligonucleotide (ISIS 440762). Further, ISIS 661134, which comprises the phosphodiester linked GalNAc₃-2 conjugate at the 5′ terminus was equipotent compared to ISIS 651900, which comprises the GalNAc₃-1 conjugate at the 3′ terminus.

TABLE 35
ASOs containing GalNAc₃-1 or GalNAc₃-2 targeting SRB-1

| | | SRB-1 | | | |
|---|---|---|---|---|---|
| ISIS | Dosage | mRNA levels | ED₅₀ | | SEQ |
| No. | (mg/kg) | (% PBS) | (mg/kg) | Conjugate | ID No. |
| PBS | 0 | 100 | — | — | |
| 440762 | 0.2 | 116 | 2.58 | No conjugate | 823 |
| | 0.7 | 91 | | | |
| | 2 | 69 | | | |
| | 7 | 22 | | | |
| | 20 | 5 | | | |
| 651900 | 0.07 | 95 | 0.26 | 3′ GalNAc3-1 | 824 |
| | 0.2 | 77 | | | |
| | 0.7 | 28 | | | |
| | 2 | 11 | | | |
| | 7 | 8 | | | |
| 661134 | 0.07 | 107 | 0.25 | 5′ GalNAc3-2 | 827 |
| | 0.2 | 86 | | | |
| | 0.7 | 28 | | | |
| | 2 | 10 | | | |
| | 7 | 6 | | | |

Structures for 3′ GalNAc3-1 and 5′ GalNAc3-2 were described previoulsy in Examples 9 and 37.

### Pharmacokinetics Analysis (PK)

The PK of the ASOs from the high dose group (7 mg/kg) was examined and evaluated in the same manner as illustrated in Example 20. Liver sample was minced and extracted using standard protocols. The full length metabolites of 661134 (5′ GalNAc₃-2) and ISIS 651900 (3′ GalNAc₃-1) were identified and their masses were confirmed by high resolution mass spectrometry analysis. The results showed that the major metabolite detected for the ASO comprising a phosphodiester linked GalNAc₃-2 conjugate at the 5′ terminus (ISIS 661134) was ISIS 440762 (data not shown). No additional metabolites, at a detectable level, were observed. Unlike its counterpart, additional metabolites similar to those reported previously in Table 23a were observed for the ASO having the GalNAc₃-1 conjugate at the 3′ terminus (ISIS 651900). These results suggest that having the phosphodiester linked GalNAc₃-1 or GalNAc₃-2 conjugate may improve the PK profile of ASOs without compromising their potency.

### Example 44: Effect of PO/PS Linkages on Antisense Inhibition of ASOs Comprising GalNAc₃-1 Conjugate (See Example 9) at the 3′ Terminus Targeting SRB-1

ISIS 655861 and 655862 comprising a GalNAc₃-1 conjugate at the 3′ terminus each targeting SRB-1 were tested in a single administration study for their ability to inhibit SRB-1 in mice. The parent unconjugated compound, ISIS 353382 was included in the study for comparison.

The ASOs are 5-10-5 MOE gapmers, wherein the gap region comprises ten 2′-deoxyribonucleosides and each wing region comprises five 2′-MOE modified nucleosides. The ASOs were prepared using similar methods as illustrated previously in Example 19 and are described Table 36, below.

TABLE 36
Modified ASOs comprising GalNAc₃-1
conjugate at the 3′ terminus targeting SRB-1

| | | | SEQ |
|---|---|---|---|
| | | | ID |
| ISIS No. | Sequence (5′ to 3′) | Chemistry | No. |
| 353382 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | Full PS no | 829 |
| (parent) | ^{m}C_{ds}T_{ds}Tₑₛ^{m}Cₑₛ^{m}CₑₛTₑₛTₑ | conjugate | |
| 655861 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | Full PS with | 830 |
| | ^{m}C_{ds}T_{ds}Tₑₛ^{m}Cₑₛ^{m}CₑₛTₑₛTₑₒA_{do′}-GalNAc₃-1ₐ | GalNAc₃-1 | |
| | | conjugate | |
| 655862 | Gₑₛ^{m}CₑₒTₑₒTₑₒ^{m}CₑₒA_{ds}G_{ds}T_{ds}^{m}C_{ds}A_{ds}T_{ds}G_{ds}A_{ds} | Mixed PS/PO | 830 |
| | ^{m}C_{ds}T_{ds}Tₑₒ^{m}Cₑₒ^{m}CₑₛTₑₛTₑₒA_{do′}-GalNAc₃-1ₐ | with GalNAc₃-1 | |
| | | conjugate | |

Subscripts: “e” indicates 2′-MOE modified nucleoside; “d” indicates β-D-2′-deoxyribonucleoside; “s” indicates phosphorothioate internucleoside linkages (PS); “o” indicates phosphodiester internucleoside linkages (PO); and “o′” indicates —O—P(═O)(OH)—. Superscript “m” indicates 5-methylcytosines. The structure of “GalNAc₃-1” is shown in Example 9.

### Treatment

Six week old male Balb/c mice (Jackson Laboratory, Bar Harbor, ME) were injected subcutaneously once at the dosage shown below with ISIS 353382, 655861, 655862 or with PBS treated control. Each treatment group consisted of 4 animals. Prior to the treatment as well as after the last dose, blood was drawn from each mouse and plasma samples were analyzed. The mice were sacrificed 72 hours following the final administration to determine the liver SRB-1 mRNA levels using real-time PCR and RIBOGREEN® RNA quantification reagent (Molecular Probes, Inc. Eugene, OR) according to standard protocols. SRB-1 mRNA levels were determined relative to total RNA (using Ribogreen), prior to normalization to PBS-treated control. The results below are presented as the average percent of SRB-1 mRNA levels for each treatment group, normalized to PBS-treated control and is denoted as “% PBS”. The ED_{50S} were measured using similar methods as described previously and are reported below.

As illustrated in Table 37, treatment with antisense oligonucleotides lowered SRB-1 mRNA levels in a dose-dependent manner compared to PBS treated control. Indeed, the antisense oligonucleotides comprising the GalNAc₃-1 conjugate at the 3′ terminus (ISIS 655861 and 655862) showed substantial improvement in potency comparing to the unconjugated antisense oligonucleotide (ISIS 353382). Further, ISIS 655862 with mixed PS/PO linkages showed an improvement in potency relative to full PS (ISIS 655861).

TABLE 37
Effect of PO/PS linkages on antisense inhibition of ASOs comprising
GalNAc₃-1 conjugate at 3′ terminus targeting SRB-1

| | | SRB-1 | | | |
|---|---|---|---|---|---|
| ISIS | Dosage | mRNA levels | ED₅₀ | | SEQ |
| No. | (mg/kg) | (% PBS) | (mg/kg) | Chemistry | ID No. |
| PBS | 0 | 100 | — | — | |
| 353382 | 3 | 76.65 | 10.4 | Full PS without | 829 |
| (parent) | 10 | 52.40 | | conjugate | |
| | 30 | 24.95 | | | |
| 655861 | 0.5 | 81.22 | 2.2 | Full PS with | 830 |
| | 1.5 | 63.51 | | GalNAc₃-1 | |
| | 5 | 24.61 | | conjugate | |
| | 15 | 14.80 | | | |
| 655862 | 0.5 | 69.57 | 1.3 | Mixed PS/PO | 830 |
| | 1.5 | 45.78 | | with GalNAc₃-1 | |
| | 5 | 19.70 | | conjugate | |
| | 15 | 12.90 | | | |

Liver transaminase levels, alanine aminotransferase (ALT) and aspartate aminotransferase (AST), in serum were measured relative to saline injected mice using standard protocols. Organ weights were also evaluated. The results demonstrated that no elevation in transaminase levels (Table 38) or organ weights (data not shown) were observed in mice treated with ASOs compared to PBS control. Further, the ASO with mixed PS/PO linkages (ISIS 655862) showed similar transaminase levels compared to full PS (ISIS 655861).

TABLE 38
Effect of PO/PS linkages on transaminase levels of ASOs comprising
GalNAc₃-1 conjugate at 3′ terminus targeting SRB-1

| ISIS | Dosage | ALT | AST | | SEQ |
|---|---|---|---|---|---|
| No. | (mg/kg) | (U/L) | (U/L) | Chemistry | ID No. |
| PBS | 0 | 28.5 | 65 | — | |
| 353382 | 3 | 50.25 | 89 | Full PS without | 829 |
| (parent) | 10 | 27.5 | 79.3 | conjugate | |
| | 30 | 27.3 | 97 | | |
| 655861 | 0.5 | 28 | 55.7 | Full PS with | 830 |
| | 1.5 | 30 | 78 | GalNAc3-1 | |
| | 5 | 29 | 63.5 | | |
| | 15 | 28.8 | 67.8 | | |
| 655862 | 0.5 | 50 | 75.5 | Mixed PS/PO | 830 |
| | 1.5 | 21.7 | 58.5 | with | |
| | 5 | 29.3 | 69 | GalNAc3-1 | |
| | 15 | 22 | 61 | | |

### Example 45: Preparation of PFP Ester, Compound 110a

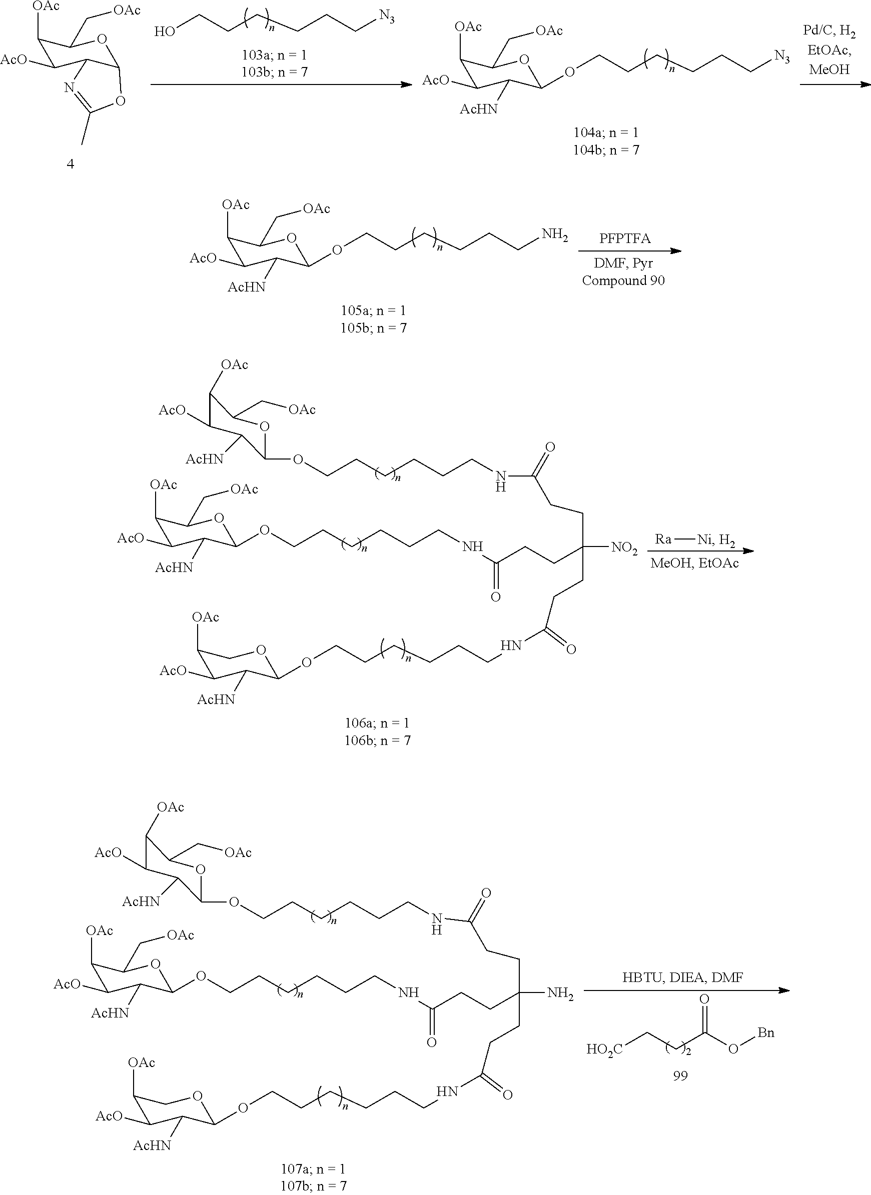

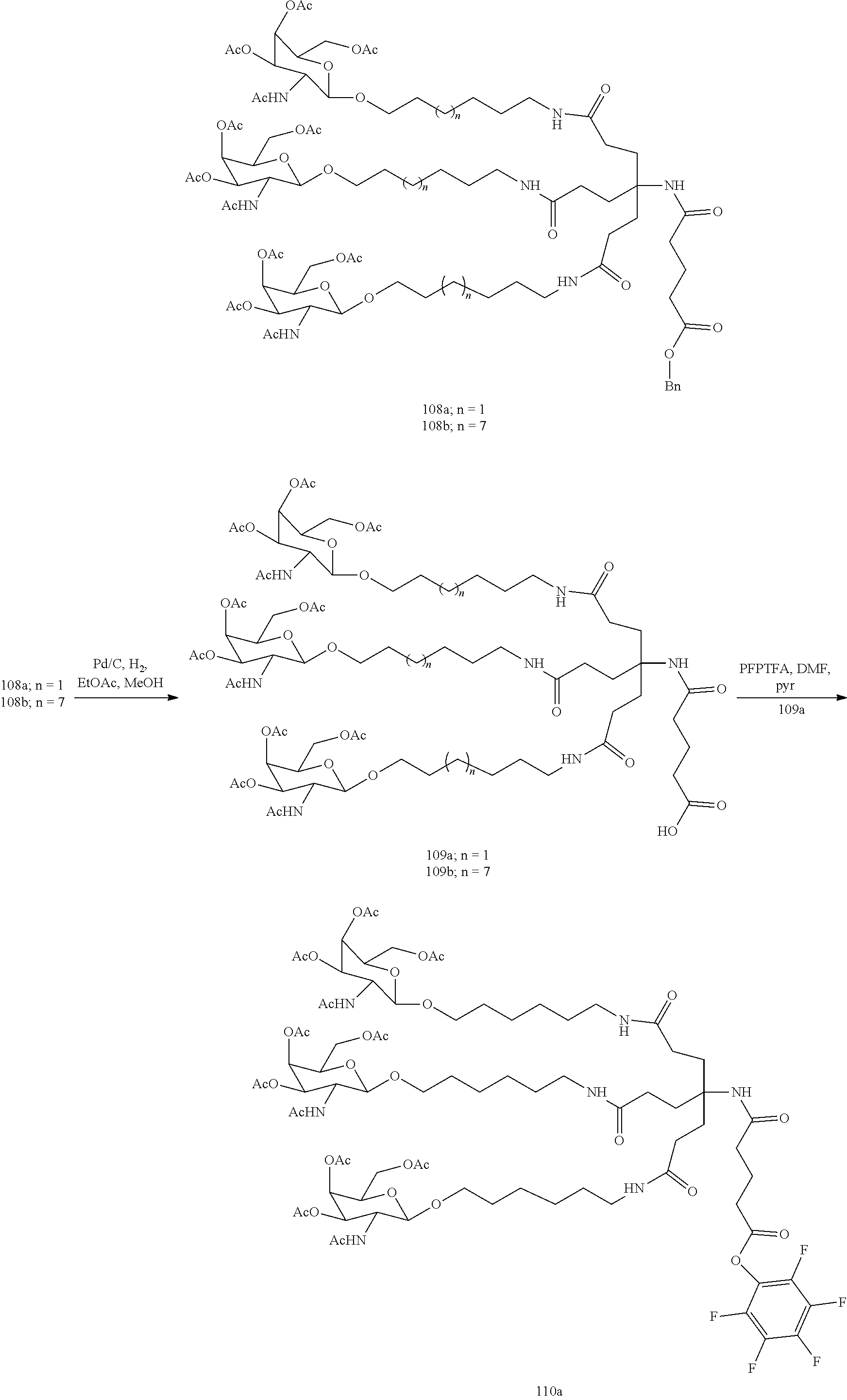

Compound 4 (9.5 g, 28.8 mmoles) was treated with compound 103a or 103b (38 mmoles), individually, and TMSOTf (0.5 eq.) and molecular sieves in dichloromethane (200 mL), and stirred for 16 hours at room temperature. At that time, the organic layer was filtered thru celite, then washed with sodium bicarbonate, water and brine. The organic layer was then separated and dried over sodium sulfate, filtered and reduced under reduced pressure. The resultant oil was purified by silica gel chromatography (2%-->10% methanol/dichloromethane) to give compounds 104a and 104b in >80% yield. LCMS and proton NMR was consistent with the structure.

Compounds 104a and 104b were treated to the same conditions as for compounds 100a-d (Example 47), to give compounds 105a and 105b in >90% yield. LCMS and proton NMR was consistent with the structure.

Compounds 105a and 105b were treated, individually, with compound 90 under the same conditions as for compounds 901a-d, to give compounds 106a (80%) and 106b (20%). LCMS and proton NMR was consistent with the structure.

Compounds 106a and 106b were treated to the same conditions as for compounds 96a-d (Example 47), to give 107a (60%) and 107b (20%). LCMS and proton NMR was consistent with the structure.

Compounds 107a and 107b were treated to the same conditions as for compounds 97a-d (Example 47), to give compounds 108a and 108b in 40-60% yield. LCMS and proton NMR was consistent with the structure.

Compounds 108a (60%) and 108b (40%) were treated to the same conditions as for compounds 100a-d (Example 47), to give compounds 109a and 109b in >80% yields. LCMS and proton NMR was consistent with the structure.

Compound 109a was treated to the same conditions as for compounds 101a-d (Example 47), to give Compound 110a in 30-60% yield. LCMS and proton NMR was consistent with the structure. Alternatively, Compound 110b can be prepared in a similar manner starting with Compound 109b.

### Example 46: General Procedure for Conjugation with PFP Esters (Oligonucleotide 111); Preparation of ISIS 666881 (GalNAc₃-10)

A 5′-hexylamino modified oligonucleotide was synthesized and purified using standard solid-phase oligonucleotide procedures. The 5′-hexylamino modified oligonucleotide was dissolved in 0.1 M sodium tetraborate, pH 8.5 (200 μL) and 3 equivalents of a selected PFP esterified GalNAc3 cluster dissolved in DMSO (50 μL) was added. If the PFP ester precipitated upon addition to the ASO solution DMSO was added until all PFP ester was in solution. The reaction was complete after about 16 h of mixing at room temperature. The resulting solution was diluted with water to 12 mL and then spun down at 3000 rpm in a spin filter with a mass cut off of 3000 Da. This process was repeated twice to remove small molecule impurities. The solution was then lyophilized to dryness and redissolved in concentrated aqueous ammonia and mixed at room temperature for 2.5 h followed by concentration in vacuo to remove most of the ammonia. The conjugated oligonucleotide was purified and desalted by RP-HPLC and lyophilized to provide the GalNAc3 conjugated oligonucleotide.

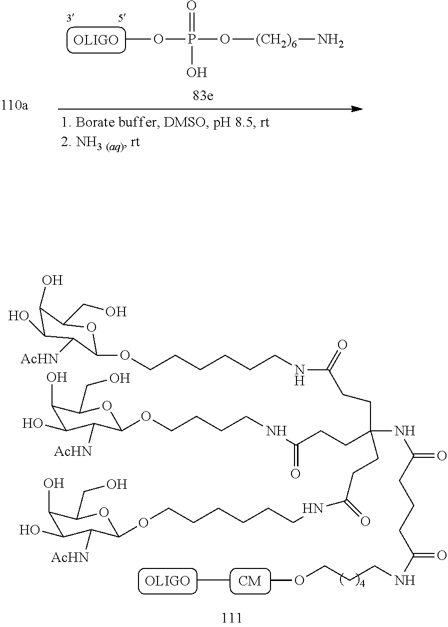

Oligonucleotide 111 is conjugated with GalNAc₃-10. The GalNAc3 cluster portion of the conjugate group GalNAc₃-10 (GalNAc₃-10ₐ) can be combined with any cleavable moiety to provide a variety of conjugate groups. In certain embodiments, the cleavable moiety is —P(═O)(OH)-A_{d}-P(═O)(OH)— as shown in the oligonucleotide (ISIS 666881) synthesized with GalNAc₃-10 below. The structure of GalNAc₃-10 (GalNAc₃-10ₐ-CM-) is shown below:

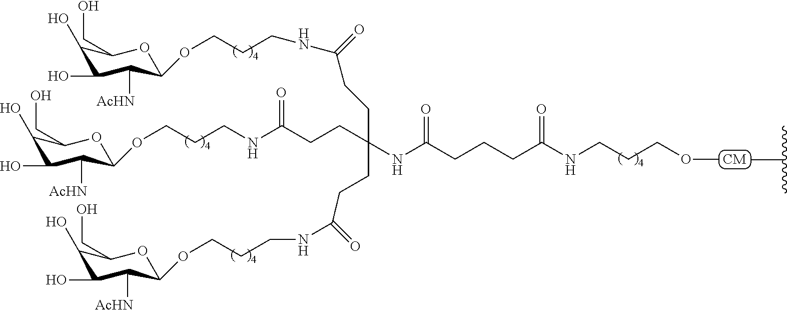

Following this general procedure ISIS 666881 was prepared. 5′-hexylamino modified oligonucleotide, ISIS 660254, was synthesized and purified using standard solid-phase oligonucleotide procedures. ISIS 660254 (40 mg, 5.2 μmol) was dissolved in 0.1 M sodium tetraborate, pH 8.5 (200 μL) and 3 equivalents PFP ester (Compound 110a) dissolved in DMSO (50 μL) was added. The PFP ester precipitated upon addition to the ASO solution requiring additional DMSO (600 μL) to fully dissolve the PFP ester. The reaction was complete after 16 h of mixing at room temperature. The solution was diluted with water to 12 mL total volume and spun down at 3000 rpm in a spin filter with a mass cut off of 3000 Da. This process was repeated twice to remove small molecule impurities. The solution was lyophilized to dryness and redissolved in concentrated aqueous ammonia with mixing at room temperature for 2.5 h followed by concentration in vacuo to remove most of the ammonia. The conjugated oligonucleotide was purified and desalted by RP-HPLC and lyophilized to give ISIS 666881 in 90% yield by weight (42 mg, 4.7 μmol).

GalNAc₃-10 conjugated oligonucleotide

| | | | SEQ |
|---|---|---|---|
| ASO | Sequence (5′ to 3′) | 5′ group | ID No. |
| ISIS 660254 | NH₂(CH₂)₆₋ₒ | Hexylamine | 831 |
| | A_{do}Gₑₛ^{m}CₑₛTₑₛTₑₛ^{m}CₑₛA_{ds}G_{ds}T_{ds} | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | |
| ISIS 666881 | GalNAc₃-10ₐ-_{o′} | GalNAc₃-10 | 831 |
| | A_{do}Gₑₛ^{m}CₑₛTₑₛTₑₛ^{m}CₑₛA_{ds}G_{ds}T_{ds} | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | |

Capital letters indicate the nucleobase for each nucleoside and ^{m}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

### Example 47: Preparation of Oligonucleotide 102 Comprising GalNAc₃-8

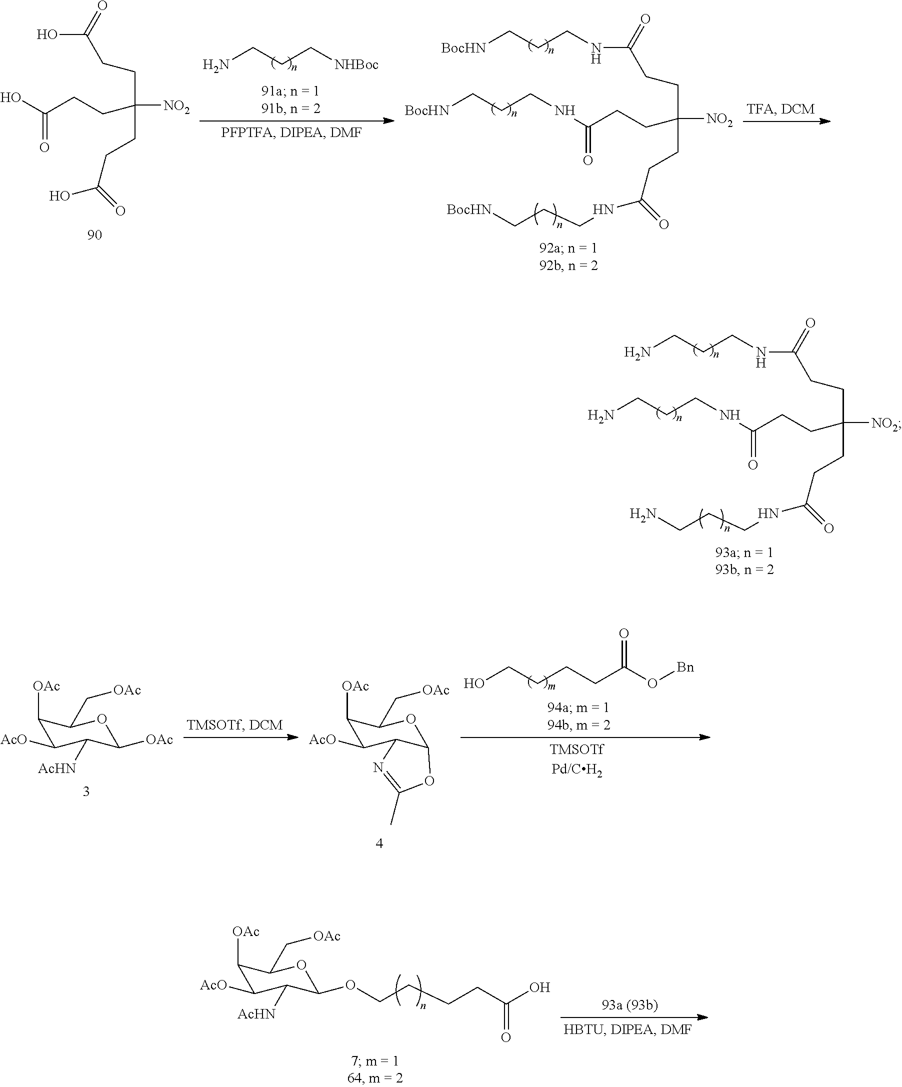

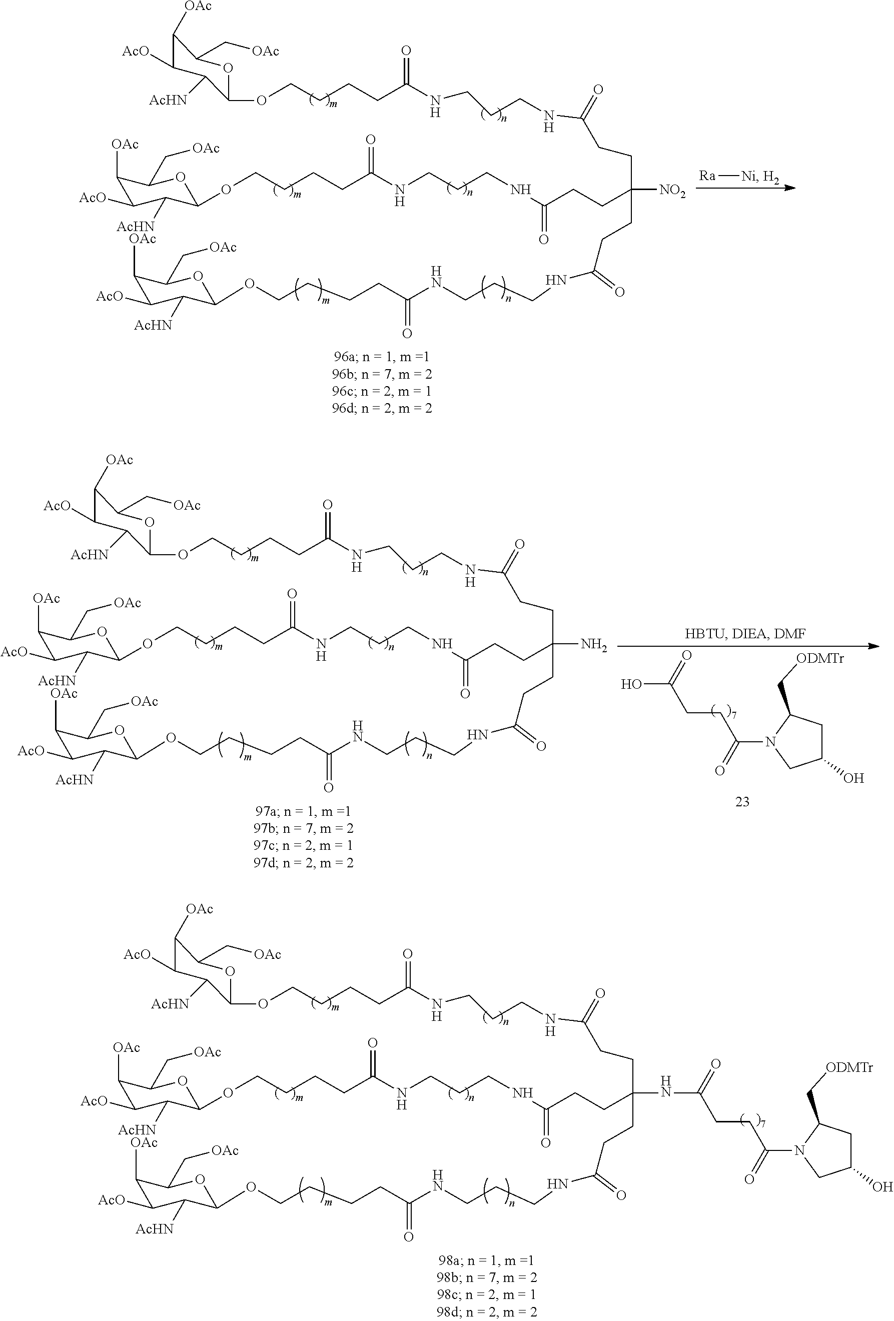

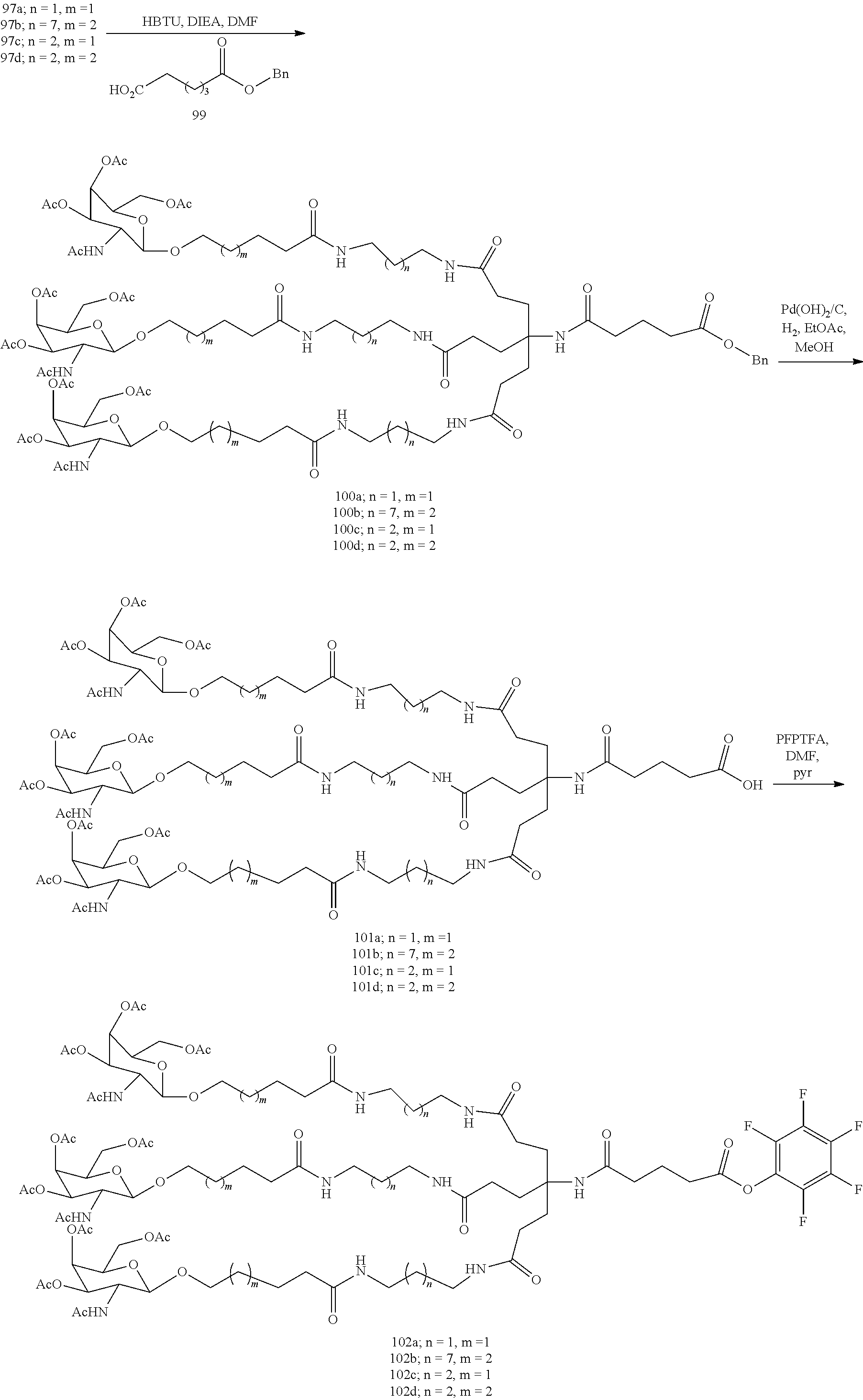

The triacid 90 (4 g, 14.43 mmol) was dissolved in DMF (120 mL) and N,N-Diisopropylethylamine (12.35 mL, 72 mmoles). Pentafluorophenyl trifluoroacetate (8.9 mL, 52 mmoles) was added dropwise, under argon, and the reaction was allowed to stir at room temperature for 30 minutes. Boc-diamine 91a or 91b (68.87 mmol) was added, along with N,N-Diisopropylethylamine (12.35 mL, 72 mmoles), and the reaction was allowed to stir at room temperature for 16 hours. At that time, the DMF was reduced by >75% under reduced pressure, and then the mixture was dissolved in dichloromethane. The organic layer was washed with sodium bicarbonate, water and brine. The organic layer was then separated and dried over sodium sulfate, filtered and reduced to an oil under reduced pressure. The resultant oil was purified by silica gel chromatography (2%-->10% methanol/dichloromethane) to give compounds 92a and 92b in an approximate 80% yield. LCMS and proton NMR were consistent with the structure.

Compound 92a or 92b (6.7 mmoles) was treated with 20 mL of dichloromethane and 20 mL of trifluoroacetic acid at room temperature for 16 hours. The resultant solution was evaporated and then dissolved in methanol and treated with DOWEX-OH resin for 30 minutes. The resultant solution was filtered and reduced to an oil under reduced pressure to give 85-90% yield of compounds 93a and 93b.

Compounds 7 or 64 (9.6 mmoles) were treated with HBTU (3.7 g, 9.6 mmoles) and N,N-Diisopropylethylamine (5 mL) in DMF (20 mL) for 15 minutes. To this was added either compounds 93a or 93b (3 mmoles), and allowed to stir at room temperature for 16 hours. At that time, the DMF was reduced by >75% under reduced pressure, and then the mixture was dissolved in dichloromethane.

The organic layer was washed with sodium bicarbonate, water and brine. The organic layer was then separated and dried over sodium sulfate, filtered and reduced to an oil under reduced pressure. The resultant oil was purified by silica gel chromatography (5%-->20% methanol/dichloromethane) to give compounds 96a-d in 20-40% yield. LCMS and proton NMR was consistent with the structure.

Compounds 96a-d (0.75 mmoles), individually, were hydrogenated over Raney Nickel for 3 hours in Ethanol (75 mL). At that time, the catalyst was removed by filtration thru celite, and the ethanol removed under reduced pressure to give compounds 97a-d in 80-90% yield. LCMS and proton NMR were consistent with the structure.

Compound 23 (0.32 g, 0.53 mmoles) was treated with HBTU (0.2 g, 0.53 mmoles) and N,N-Diisopropylethylamine (0.19 mL, 1.14 mmoles) in DMF (30 mL) for 15 minutes. To this was added compounds 97a-d (0.38 mmoles), individually, and allowed to stir at room temperature for 16 hours.

At that time, the DMF was reduced by >75% under reduced pressure, and then the mixture was dissolved in dichloromethane. The organic layer was washed with sodium bicarbonate, water and brine. The organic layer was then separated and dried over sodium sulfate, filtered and reduced to an oil under reduced pressure. The resultant oil was purified by silica gel chromatography (2%-->20% methanol/dichloromethane) to give compounds 98a-d in 30-40% yield. LCMS and proton NMR was consistent with the structure.

Compound 99 (0.17 g, 0.76 mmoles) was treated with HBTU (0.29 g, 0.76 mmoles) and N,N-Diisopropylethylamine (0.35 mL, 2.0 mmoles) in DMF (50 mL) for 15 minutes. To this was added compounds 97a-d (0.51 mmoles), individually, and allowed to stir at room temperature for 16 hours.

At that time, the DMF was reduced by >75% under reduced pressure, and then the mixture was dissolved in dichloromethane. The organic layer was washed with sodium bicarbonate, water and brine. The organic layer was then separated and dried over sodium sulfate, filtered and reduced to an oil under reduced pressure. The resultant oil was purified by silica gel chromatography (5%-->20% methanol/dichloromethane) to give compounds 100a-d in 40-60% yield. LCMS and proton NMR was consistent with the structure.

Compounds 100a-d (0.16 mmoles), individually, were hydrogenated over 10% Pd(OH)₂/C for 3 hours in methanol/ethyl acetate (1:1, 50 mL). At that time, the catalyst was removed by filtration thru celite, and the organics removed under reduced pressure to give compounds 101a-d in 80-90% yield. LCMS and proton NMR was consistent with the structure.

Compounds 101a-d (0.15 mmoles), individually, were dissolved in DMF (15 mL) and pyridine (0.016 mL, 0.2 mmoles). Pentafluorophenyl trifluoroacetate (0.034 mL, 0.2 mmoles) was added dropwise, under argon, and the reaction was allowed to stir at room temperature for 30 minutes. At that time, the DMF was reduced by >75% under reduced pressure, and then the mixture was dissolved in dichloromethane. The organic layer was washed with sodium bicarbonate, water and brine. The organic layer was then separated and dried over sodium sulfate, filtered and reduced to an oil under reduced pressure. The resultant oil was purified by silica gel chromatography (2%-->5% methanol/dichloromethane) to give compounds 102a-d in an approximate 80% yield. LCMS and proton NMR were consistent with the structure.

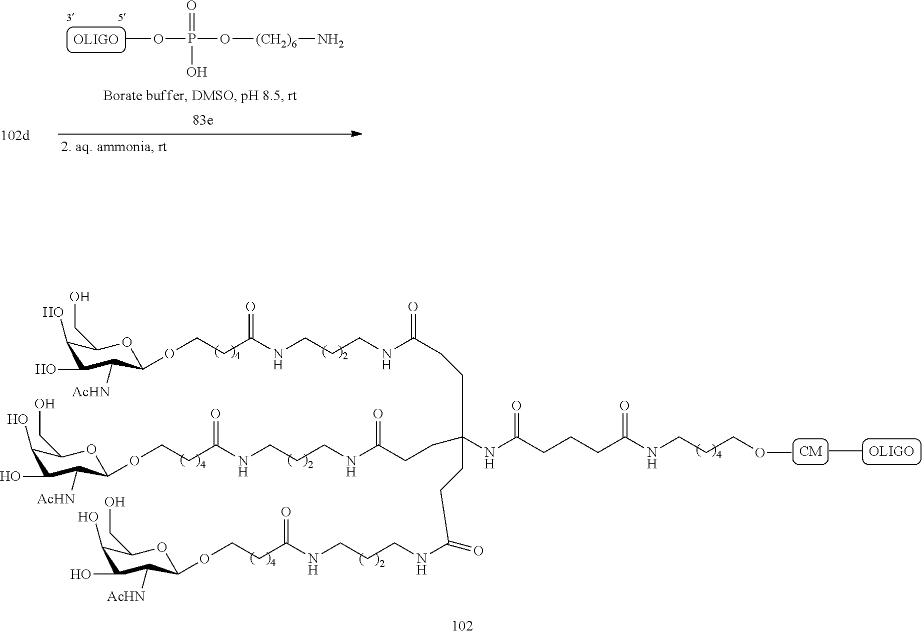

Oligomeric Compound 102, comprising a GalNAc₃-8 conjugate group, was prepared using the general procedures illustrated in Example 46. The GalNAc3 cluster portion of the conjugate group GalNAc₃-8 (GalNAc₃-8ₐ) can be combined with any cleavable moiety to provide a variety of conjugate groups. In a preferred embodiment, the cleavable moiety is —P(═O)(OH)-A_{d}-P(═O)(OH)—.

The structure of GalNAc₃-8 (GalNAc₃-8ₐ-CM-) is shown below:

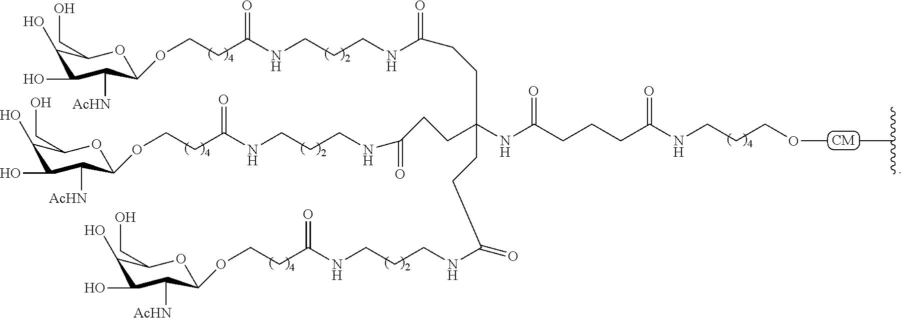

### Example 48: Preparation of Oligonucleotide 119 Comprising GalNAc₃-7

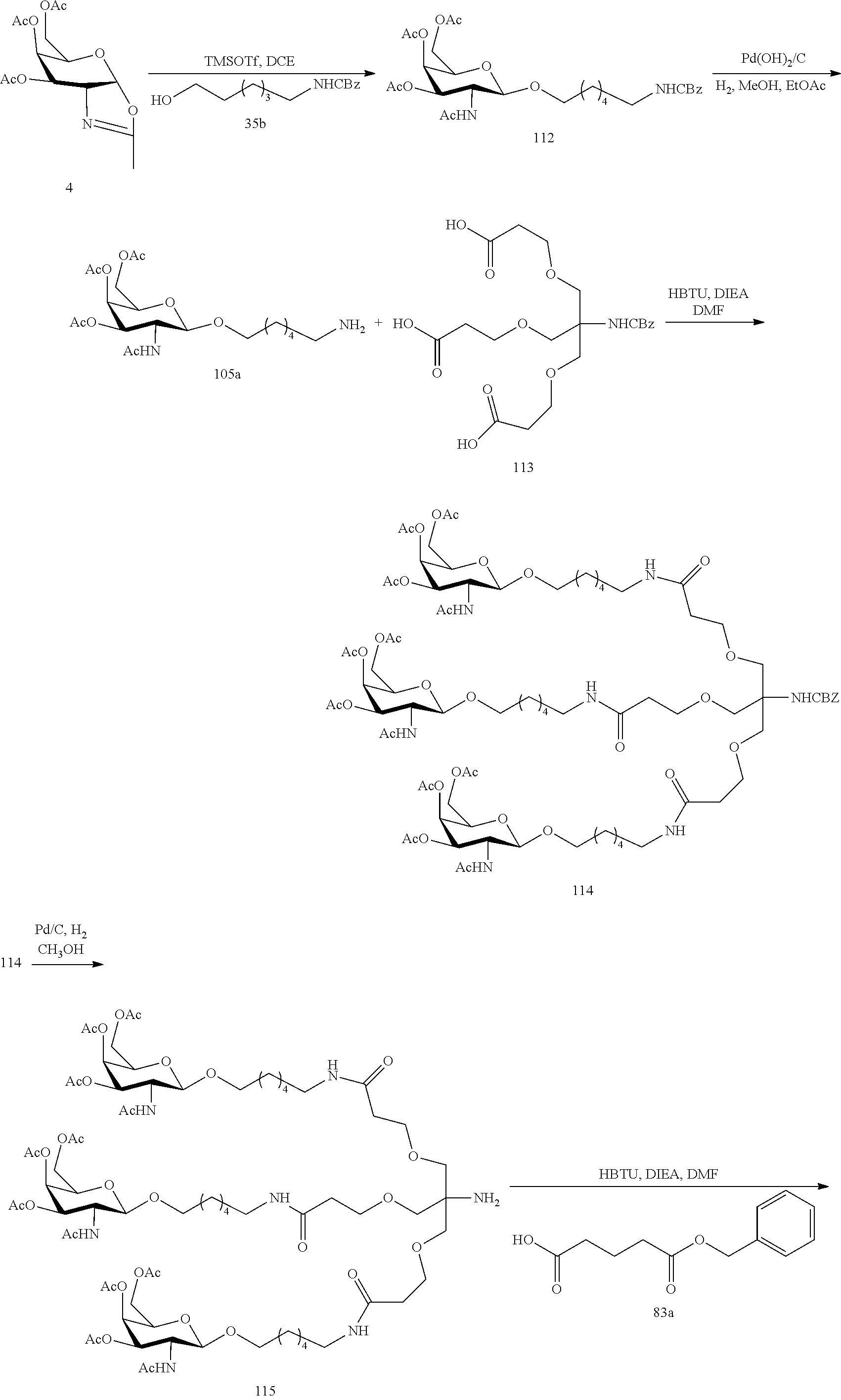

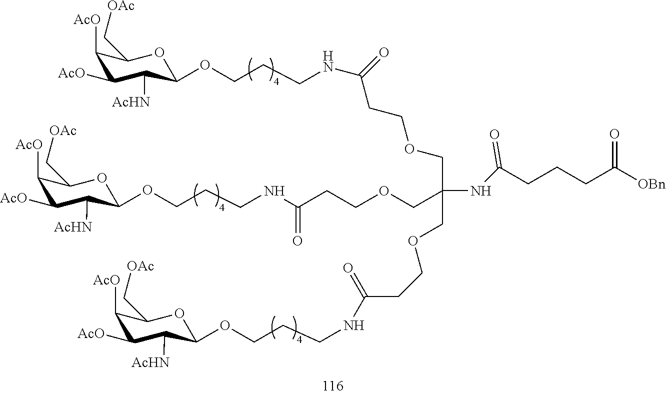

Compound 112 was synthesized following the procedure described in the literature (J. Med. Chem. 2004, 47, 5798-5808).

Compound 112 (5 g, 8.6 mmol) was dissolved in 1:1 methanol/ethyl acetate (22 mL/22 mL). Palladium hydroxide on carbon (0.5 g) was added. The reaction mixture was stirred at room temperature under hydrogen for 12 h. The reaction mixture was filtered through a pad of celite and washed the pad with 1:1 methanol/ethyl acetate. The filtrate and the washings were combined and concentrated to dryness to yield Compound 105a (quantitative). The structure was confirmed by LCMS.

Compound 113 (1.25 g, 2.7 mmol), HBTU (3.2 g, 8.4 mmol) and DIEA (2.8 mL, 16.2 mmol) were dissolved in anhydrous DMF (17 mL) and the reaction mixture was stirred at room temperature for 5 min. To this a solution of Compound 105a (3.77 g, 8.4 mmol) in anhydrous DMF (20 mL) was added. The reaction was stirred at room temperature for 6 h. Solvent was removed under reduced pressure to get an oil. The residue was dissolved in CH2C2 (100 mL) and washed with aqueous saturated NaHCO₃ solution (100 mL) and brine (100 mL). The organic phase was separated, dried (Na₂SO₄), filtered and evaporated. The residue was purified by silica gel column chromatography and eluted with 10 to 20% MeOH in dichloromethane to yield Compound 114 (1.45 g, 30%). The structure was confirmed by LCMS and ¹H NMR analysis.

Compound 114 (1.43 g, 0.8 mmol) was dissolved in 1:1 methanol/ethyl acetate (4 mL/4 mL). Palladium on carbon (wet, 0.14 g) was added. The reaction mixture was flushed with hydrogen and stirred at room temperature under hydrogen for 12 h. The reaction mixture was filtered through a pad of celite. The celite pad was washed with methanol/ethyl acetate (1:1). The filtrate and the washings were combined together and evaporated under reduced pressure to yield Compound 115 (quantitative). The structure was confirmed by LCMS and ¹H NMR analysis.

Compound 83a (0.17 g, 0.75 mmol), HBTU (0.31 g, 0.83 mmol) and DIEA (0.26 mL, 1.5 mmol) were dissolved in anhydrous DMF (5 mL) and the reaction mixture was stirred at room temperature for 5 min. To this a solution of Compound 115 (1.22 g, 0.75 mmol) in anhydrous DMF was added and the reaction was stirred at room temperature for 6 h. The solvent was removed under reduced pressure and the residue was dissolved in CH2C2. The organic layer was washed aqueous saturated NaHCO₃ solution and brine and dried over anhydrous Na₂SO₄ and filtered. The organic layer was concentrated to dryness and the residue obtained was purified by silica gel column chromatography and eluted with 3 to 15% MeOH in dichloromethane to yield Compound 116 (0.84 g, 61%). The structure was confirmed by LC MS and ¹H NMR analysis.

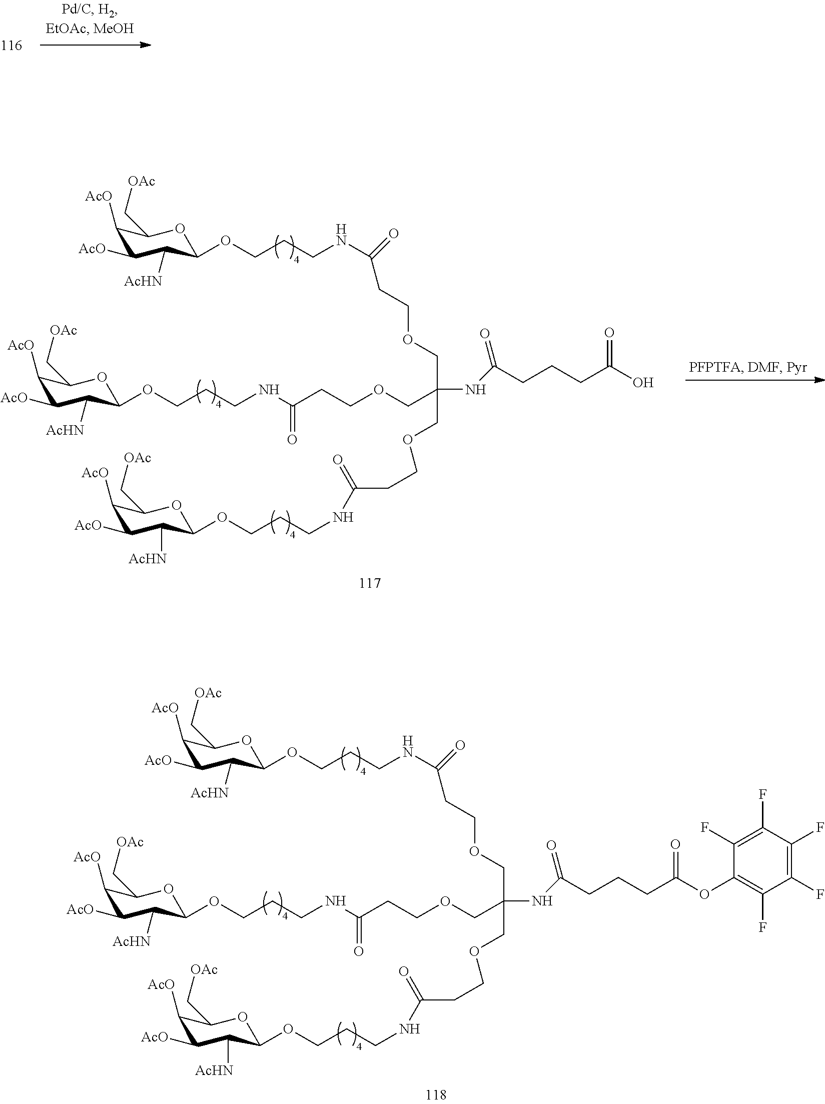

Compound 116 (0.74 g, 0.4 mmol) was dissolved in 1:1 methanol/ethyl acetate (5 mL/5 mL). Palladium on carbon (wet, 0.074 g) was added. The reaction mixture was flushed with hydrogen and stirred at room temperature under hydrogen for 12 h. The reaction mixture was filtered through a pad of celite. The celite pad was washed with methanol/ethyl acetate (1:1). The filtrate and the washings were combined together and evaporated under reduced pressure to yield compound 117 (0.73 g, 98%). The structure was confirmed by LCMS and ¹H NMR analysis.

Compound 117 (0.63 g, 0.36 mmol) was dissolved in anhydrous DMF (3 mL). To this solution N,N-Diisopropylethylamine (70 μL, 0.4 mmol) and pentafluorophenyl trifluoroacetate (72 μL, 0.42 mmol) were added. The reaction mixture was stirred at room temperature for 12 h and poured into a aqueous saturated NaHCO₃ solution. The mixture was extracted with dichloromethane, washed with brine and dried over anhydrous Na₂SO₄. The dichloromethane solution was concentrated to dryness and purified with silica gel column chromatography and eluted with 5 to 10% MeOH in dichloromethane to yield compound 118 (0.51 g, 79%). The structure was confirmed by LCMS and ¹H and ¹H and ¹⁹F NMR.

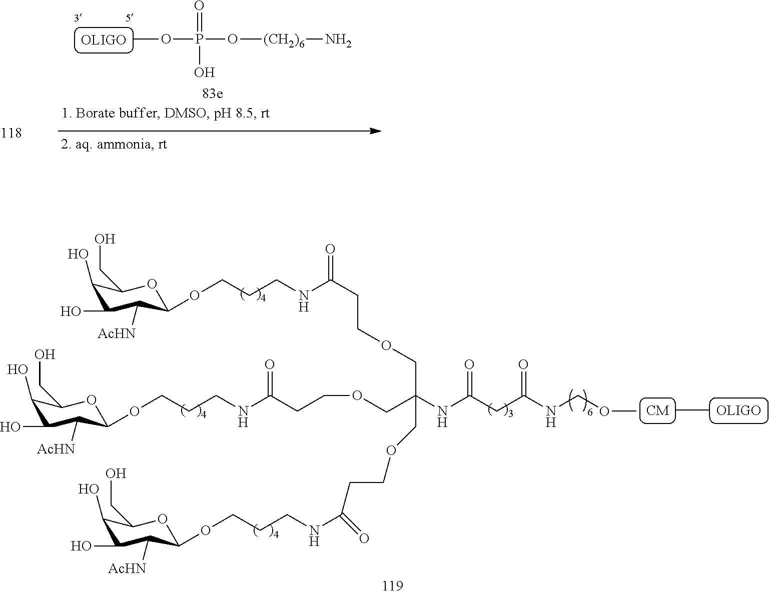

Oligomeric Compound 119, comprising a GalNAc₃-7 conjugate group, was prepared using the general procedures illustrated in Example 46. The GalNAc3 cluster portion of the conjugate group GalNAc₃-7 (GalNAc₃-7ₐ) can be combined with any cleavable moiety to provide a variety of conjugate groups. In certain embodiments, the cleavable moiety is —P(═O)(OH)-A_{d}-P(═O)(OH)—.

The structure of GalNAc₃-7 (GalNAc₃-7ₐ-CM-) is shown below:

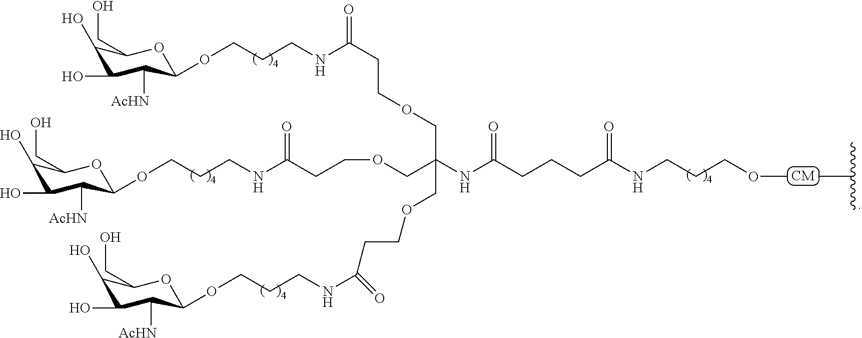

### Example 49: Preparation of Oligonucleotide 132 Comprising GalNAc₃-5

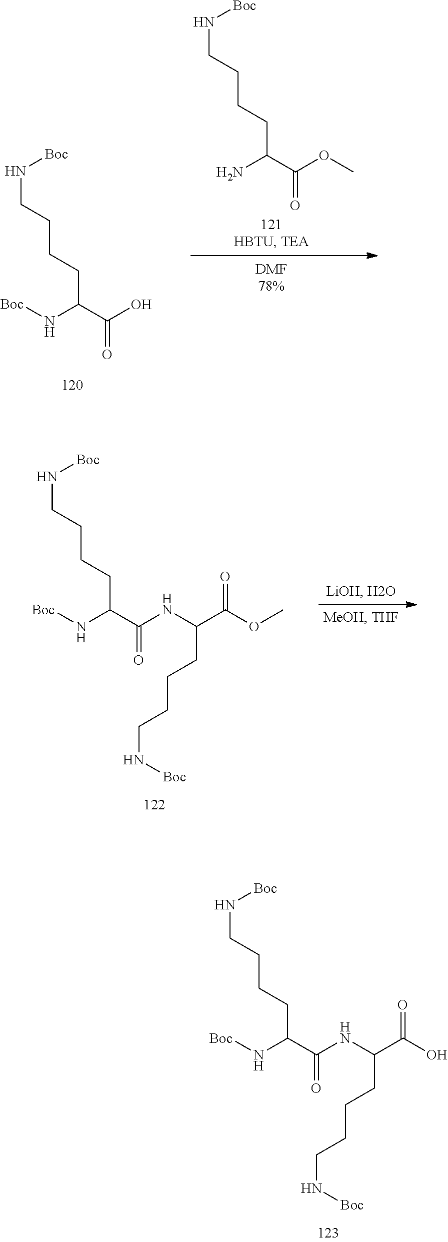 [SMILES: CC(C)(C)OC(=O)NCCCCC(NC(=O)OC(C)(C)C)C(=O)O ; COC(=O)C(N)CCCCNC(=O)OC(C)(C)C ; COC(=O)C(CCCCNC(=O)OC(C)(C)C)NC(=O)C(CCCCNC(=O)OC(C)(C)C)NC(=O)OC(C)(C)C ; CC(C)(C)OC(=O)NCCCCC(NC(=O)C(CCCCNC(=O)OC(C)(C)C)NC(=O)OC(C)(C)C)C(=O)O]

Compound 120 (14.01 g, 40 mmol) and HBTU (14.06 g, 37 mmol) were dissolved in anhydrous DMF (80 mL). Triethylamine (11.2 mL, 80.35 mmol) was added and stirred for 5 min. The reaction mixture was cooled in an ice bath and a solution of compound 121 (10 g, mmol) in anhydrous DMF (20 mL) was added. Additional triethylamine (4.5 mL, 32.28 mmol) was added and the reaction mixture was stirred for 18 h under an argon atmosphere. The reaction was monitored by TLC (ethyl acetate:hexane; 1:1; Rf=0.47). The solvent was removed under reduced pressure. The residue was taken up in EtOAc (300 mL) and washed with 1M NaHSO₄ (3×150 mL), aqueous saturated NaHCO₃ solution (3×150 mL) and brine (2×100 mL). Organic layer was dried with Na₂SO₄. Drying agent was removed by filtration and organic layer was concentrated by rotary evaporation. Crude mixture was purified by silica gel column chromatography and eluted by using 35-50% EtOAc in hexane to yield a compound 122 (15.50 g, 78.13%). The structure was confirmed by LCMS and ¹H NMR analysis. Mass m/z 589.3 [M+H]⁺.

A solution of LiGH (92.15 mmol) in water (20 mL) and THE (10 mL) was added to a cooled solution of Compound 122 (7.75 g, 13.16 mmol) dissolved in methanol (15 mL). The reaction mixture was stirred at room temperature for 45 min. and monitored by TLC (EtOAc:hexane; 1:1). The reaction mixture was concentrated to half the volume under reduced pressure. The remaining solution was cooled an ice bath and neutralized by adding concentrated HCl. The reaction mixture was diluted, extracted with EtOAc (120 mL) and washed with brine (100 mL). An emulsion formed and cleared upon standing overnight. The organic layer was separated dried (Na₂SO₄), filtered and evaporated to yield Compound 123 (8.42 g). Residual salt is the likely cause of excess mass. LCMS is consistent with structure. Product was used without any further purification. M.W.cal:574.36; M.W.fd:575.3 [M+H]⁺.

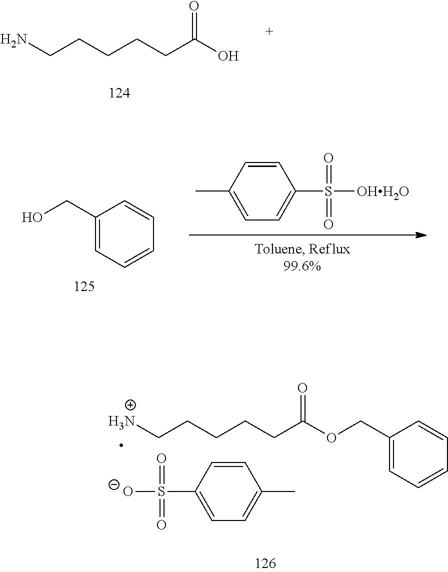

Compound 126 was synthesized following the procedure described in the literature (J. Am. Chem. Soc. 2011, 133, 958-963).

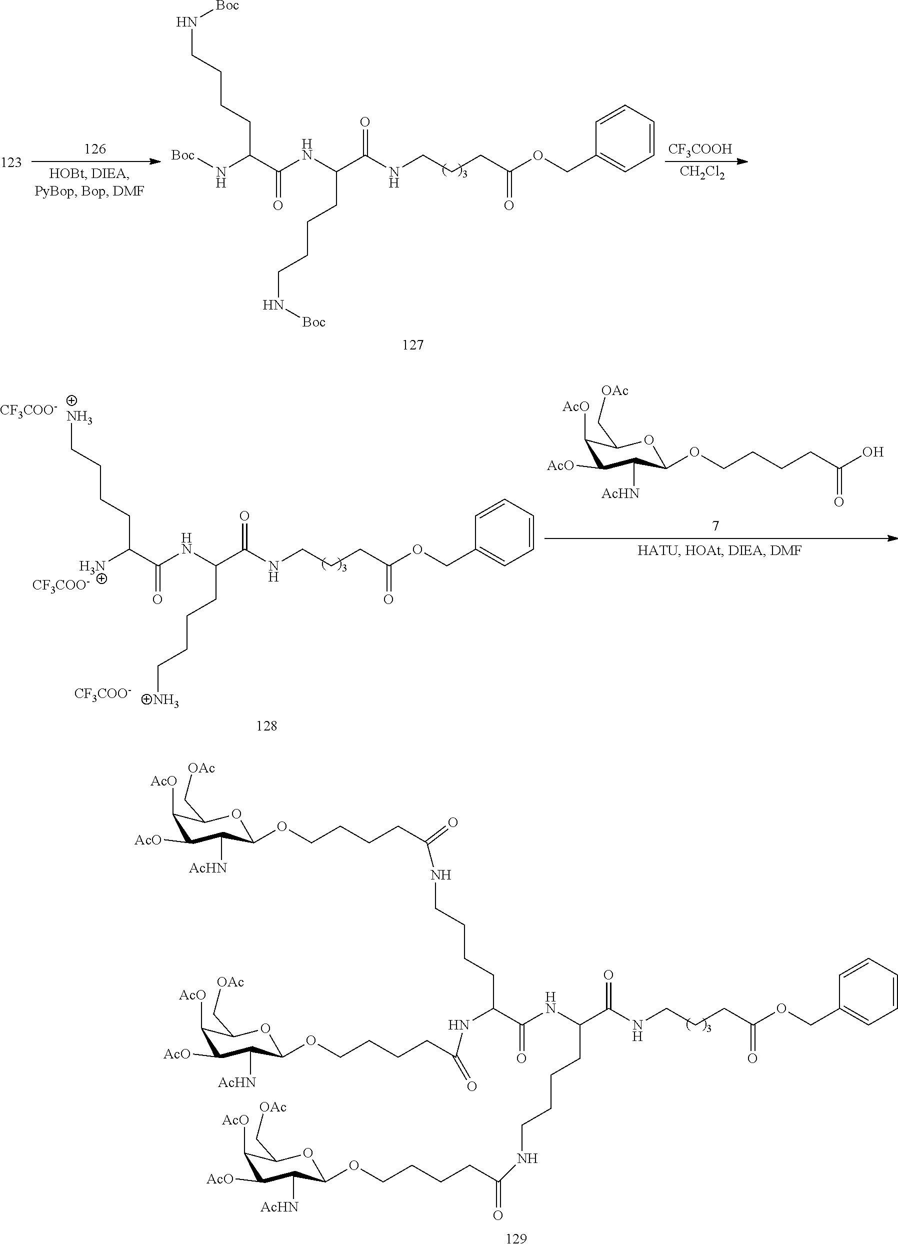

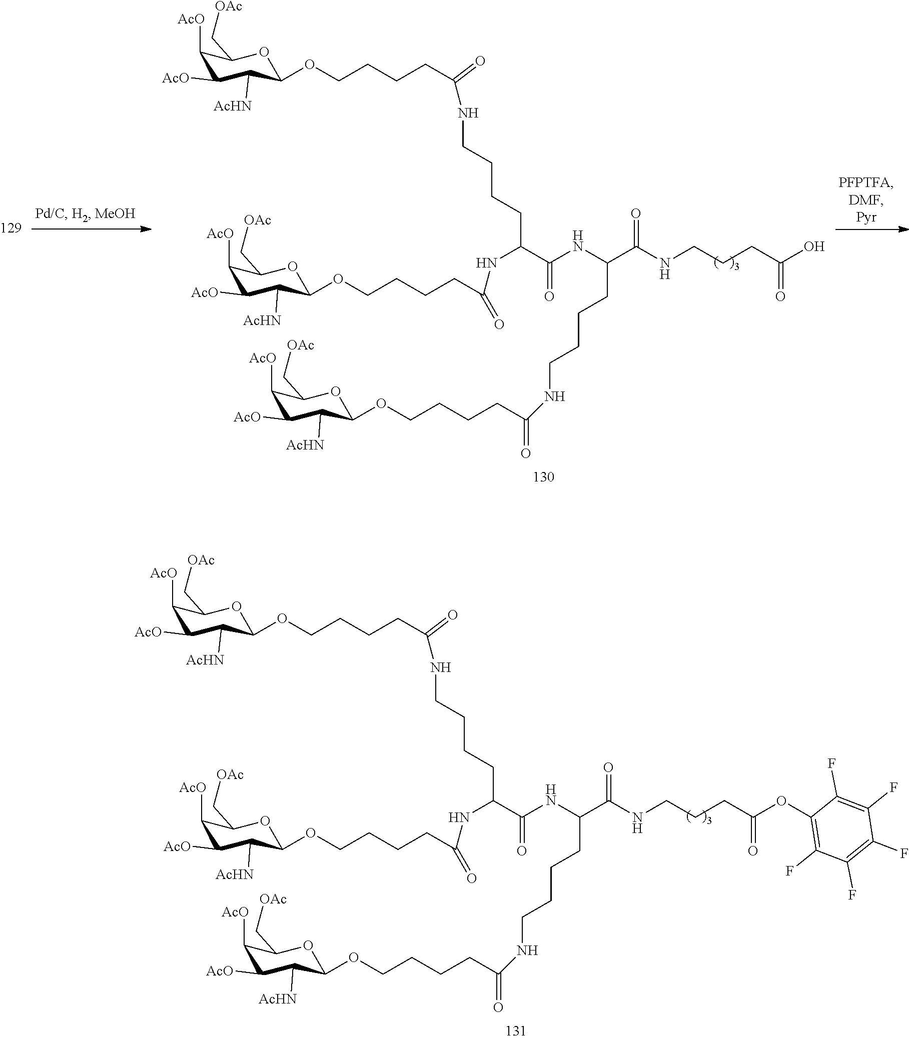

Compound 123 (7.419 g, 12.91 mmol), HOBt (3.49 g, 25.82 mmol) and compound 126 (6.33 g, 16.14 mmol) were dissolved in and DMF (40 mL) and the resulting reaction mixture was cooled in an ice bath. To this N,N-Diisopropylethylamine (4.42 mL, 25.82 mmol), PyBop (8.7 g, 16.7 mmol) followed by Bop coupling reagent (1.17 g, 2.66 mmol) were added under an argon atmosphere. The ice bath was removed and the solution was allowed to warm to room temperature. The reaction was completed after 1 h as determined by TLC (DCM:MeOH:AA; 89:10:1). The reaction mixture was concentrated under reduced pressure. The residue was dissolved in EtOAc (200 mL) and washed with 1 M NaHSO₄ (3×100 mL), aqueous saturated NaHCO₃ (3×100 mL) and brine (2×100 mL). The organic phase separated dried (Na₂SO₄), filtered and concentrated. The residue was purified by silica gel column chromatography with a gradient of 50% hexanes/EtOAC to 100% EtOAc to yield Compound 127 (9.4 g) as a white foam. LCMS and ¹H NMR were consistent with structure. Mass m/z 778.4 [M+H]⁺.

Trifluoroacetic acid (12 mL) was added to a solution of compound 127 (1.57 g, 2.02 mmol) in dichloromethane (12 mL) and stirred at room temperature for 1 h. The reaction mixture was co-evaporated with toluene (30 mL) under reduced pressure to dryness. The residue obtained was co-evaporated twice with acetonitrile (30 mL) and toluene (40 mL) to yield Compound 128 (1.67 g) as trifluoro acetate salt and used for next step without further purification. LCMS and ¹H NMR were consistent with structure. Mass m/z 478.2 [M+H]⁺.

Compound 7 (0.43 g, 0.963 mmol), HATU (0.35 g, 0.91 mmol), and HOAt (0.035 g, 0.26 mmol) were combined together and dried for 4 h over P₂O₅ under reduced pressure in a round bottom flask and then dissolved in anhydrous DMF (1 mL) and stirred for 5 min. To this a solution of compound 128 (0.20 g, 0.26 mmol) in anhydrous DMF (0.2 mL) and N,N-Diisopropylethylamine (0.2 mL) was added. The reaction mixture was stirred at room temperature under an argon atmosphere. The reaction was complete after 30 min as determined by LCMS and TLC (7% MeOH/DCM). The reaction mixture was concentrated under reduced pressure. The residue was dissolved in DCM (30 mL) and washed with 1 M NaHSO₄ (3×20 mL), aqueous saturated NaHCO₃ (3×20 mL) and brine (3×20 mL). The organic phase was separated, dried over Na₂SO₄, filtered and concentrated. The residue was purified by silica gel column chromatography using 5-15% MeOH in dichloromethane to yield Compound 129 (96.6 mg). LC MS and ¹H NMR are consistent with structure. Mass m/z 883.4 [M+2H]⁺.

Compound 129 (0.09 g, 0.051 mmol) was dissolved in methanol (5 mL) in 20 mL scintillation vial. To this was added a small amount of 10% Pd/C (0.015 mg) and the reaction vessel was flushed with H₂ gas. The reaction mixture was stirred at room temperature under H₂ atmosphere for 18 h. The reaction mixture was filtered through a pad of Celite and the Celite pad was washed with methanol. The filtrate washings were pooled together and concentrated under reduced pressure to yield Compound 130 (0.08 g). LCMS and ¹H NMR were consistent with structure. The product was used without further purification. Mass m/z 838.3 [M+2H]⁺.

To a 10 mL pointed round bottom flask were added compound 130 (75.8 mg, 0.046 mmol), 0.37 M pyridine/DMF (200 μL) and a stir bar. To this solution was added 0.7 M pentafluorophenyl trifluoroacetate/DMF (100 μL) drop wise with stirring. The reaction was completed after 1 h as determined by LC MS. The solvent was removed under reduced pressure and the residue was dissolved in CHCl₃ (˜10 mL). The organic layer was partitioned against NaHSO₄ (1 M, 10 mL), aqueous saturated NaHCO₃ (10 mL) and brine (10 mL) three times each. The organic phase separated and dried over Na₂SO₄, filtered and concentrated to yield Compound 131 (77.7 mg). LCMS is consistent with structure. Used without further purification. Mass m/z 921.3 [M+2H]⁺.

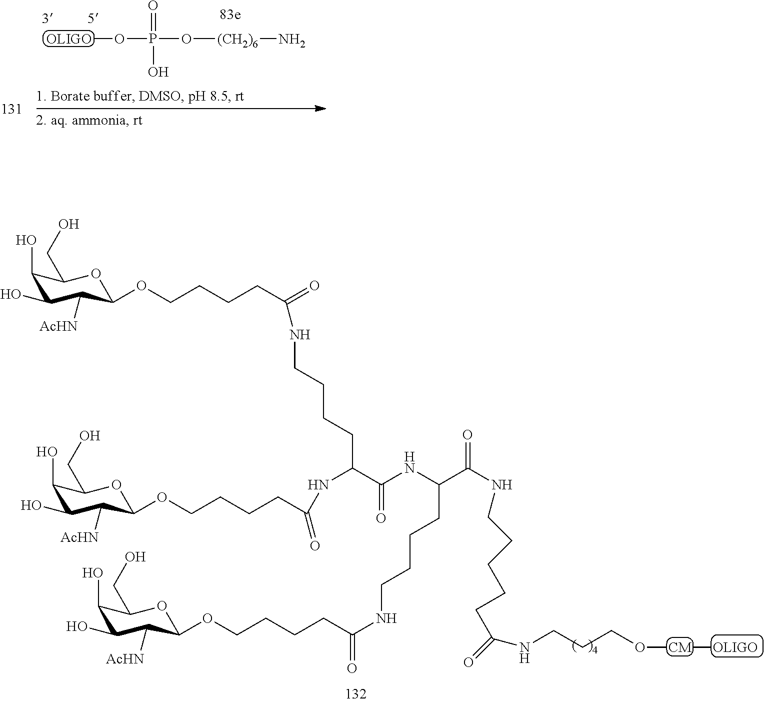

Oligomeric Compound 132, comprising a GalNAc₃-5 conjugate group, was prepared using the general procedures illustrated in Example 46. The GalNAc3 cluster portion of the conjugate group GalNAc₃-5 (GalNAc₃-5ₐ) can be combined with any cleavable moiety to provide a variety of conjugate groups. In certain embodiments, the cleavable moiety is —P(═O)(OH)-A_{d}-P(═O)(OH)—.

The structure of GalNAc₃-5 (GalNAc₃-5ₐ-CM-) is shown below:

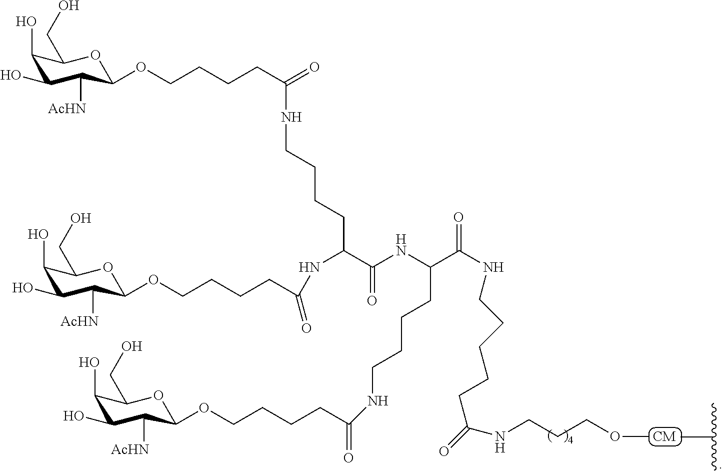

### Example 50: Preparation of Oligonucleotide 144 Comprising GalNAc₄-11

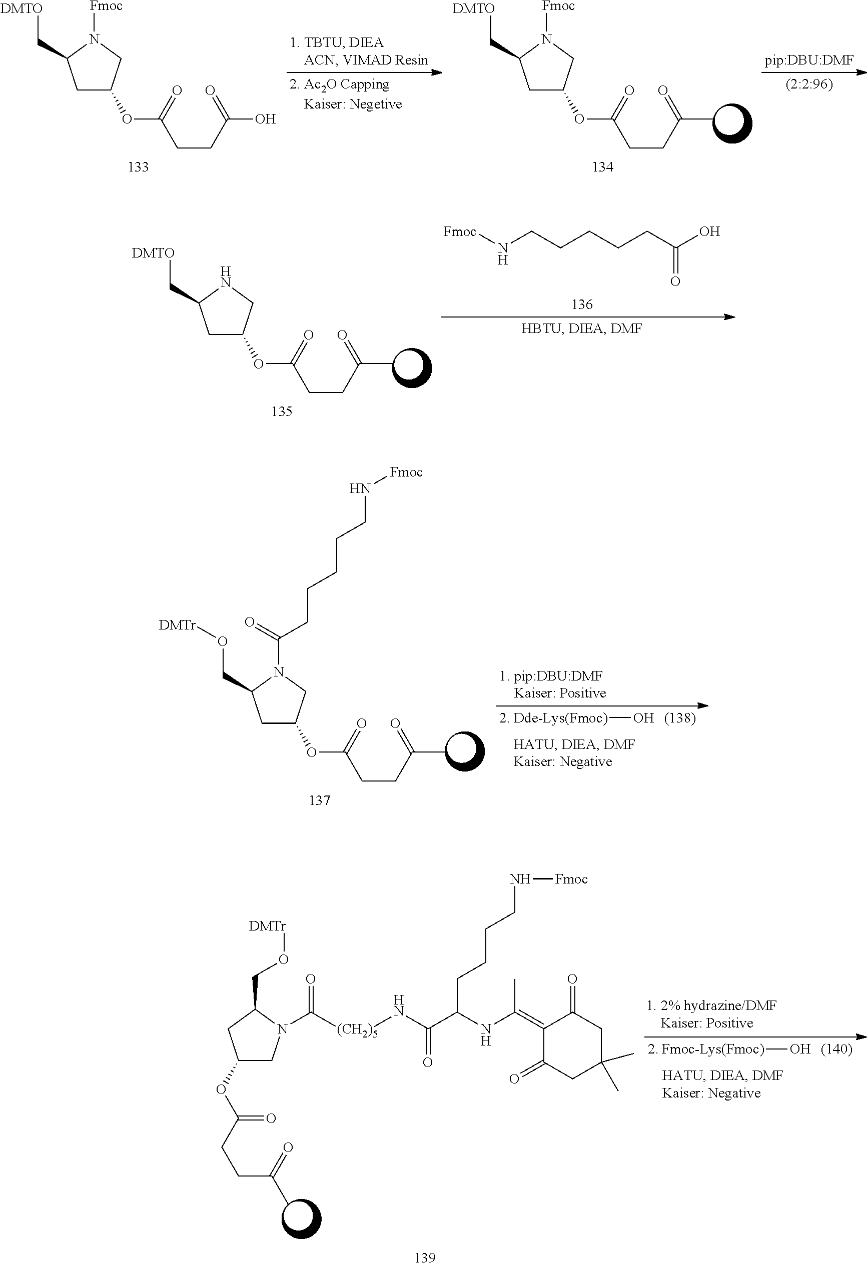

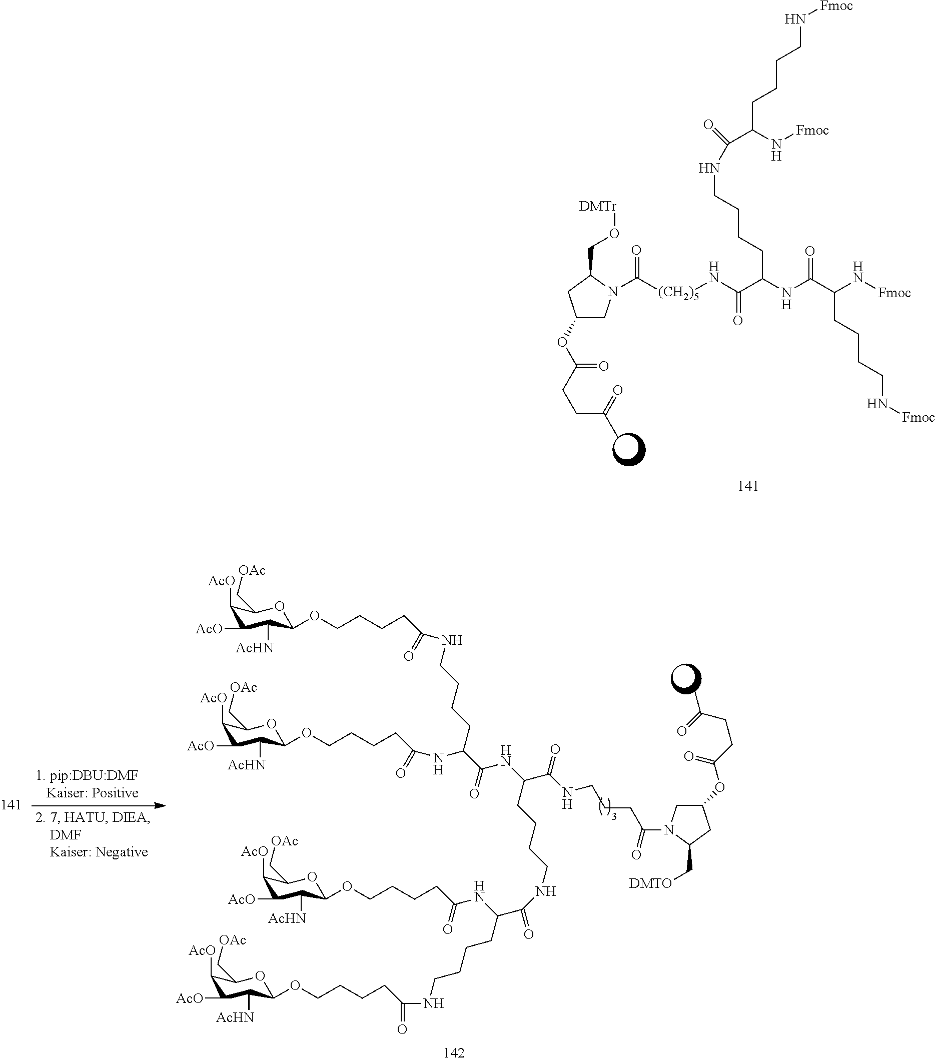

Synthesis of Compound 134. To a Merrifield flask was added aminomethyl VIMAD resin (2.5 g, 450 μmol/g) that was washed with acetonitrile, dimethylformamide, dichloromethane and acetonitrile. The resin was swelled in acetonitrile (4 mL). Compound 133 was pre-activated in a 100 mL round bottom flask by adding 20 (1.0 mmol, 0.747 g), TBTU (1.0 mmol, 0.321 g), acetonitrile (5 mL) and DIEA (3.0 mmol, 0.5 mL). This solution was allowed to stir for 5 min and was then added to the Merrifield flask with shaking. The suspension was allowed to shake for 3 h. The reaction mixture was drained and the resin was washed with acetonitrile, DMF and DCM. New resin loading was quantitated by measuring the absorbance of the DMT cation at 500 nm (extinction coefficient=76000) in DCM and determined to be 238 μmol/g. The resin was capped by suspending in an acetic anhydride solution for ten minutes three times.

The solid support bound compound 141 was synthesized using iterative Fmoc-based solid phase peptide synthesis methods. A small amount of solid support was withdrawn and suspended in aqueous ammonia (28-30 wt %) for 6 h. The cleaved compound was analyzed by LC-MS and the observed mass was consistent with structure. Mass m/z 1063.8 [M+2H]*.

The solid support bound compound 142 was synthesized using solid phase peptide synthesis methods.

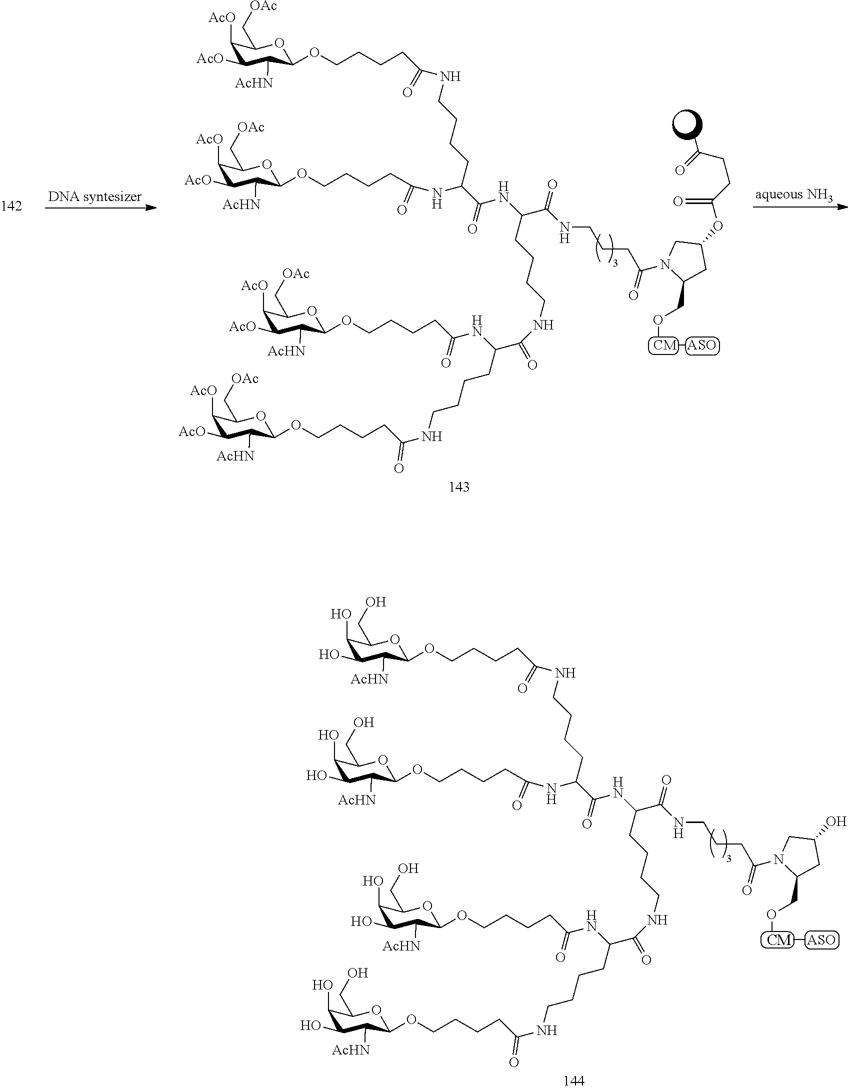

The solid support bound compound 143 was synthesized using standard solid phase synthesis on a DNA synthesizer.

The solid support bound compound 143 was suspended in aqueous ammonia (28-30 wt %) and heated at 55° C. for 16 h. The solution was cooled and the solid support was filtered. The filtrate was concentrated and the residue dissolved in water and purified by HPLC on a strong anion exchange column. The fractions containing full length compound 144 were pooled together and desalted. The resulting GalNAc₄-11 conjugated oligomeric compound was analyzed by LC-MS and the observed mass was consistent with structure.

The GalNAc4 cluster portion of the conjugate group GalNAc₄-11 (GalNAc₄-11ₐ) can be combined with any cleavable moiety to provide a variety of conjugate groups. In certain embodiments, the cleavable moiety is —P(═O)(OH)-A_{d}-P(═O)(OH)—.

The structure of GalNAc₄-11 (GalNAc₄-11ₐ-CM) is shown below:

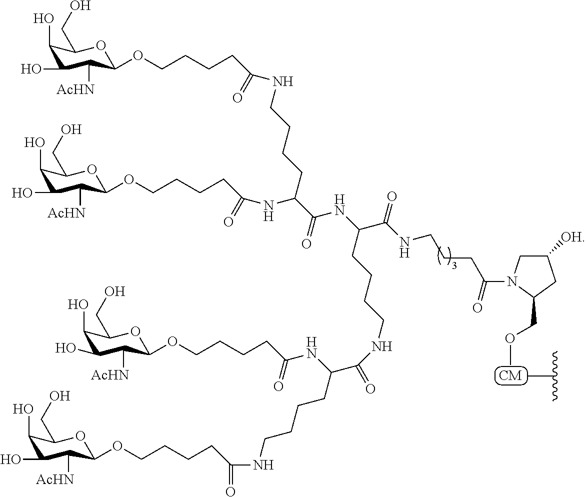

### Example 51: Preparation of Oligonucleotide 155 Comprising GalNAc₃-6

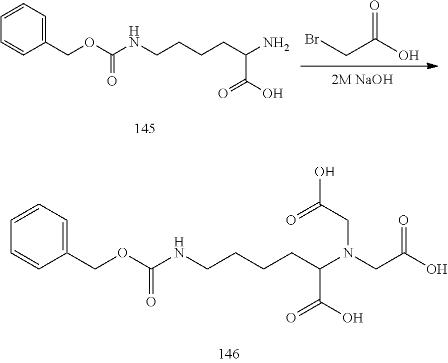 [SMILES: NC(CCCCNC(=O)OCc1ccccc1)C(=O)O ; O=C(O)CBr ; O=C(O)CN(CC(=O)O)C(CCCCNC(=O)OCc1ccccc1)C(=O)O]

Compound 146 was synthesized as described in the literature (Analytical Biochemistry 1995, 229, 54-60).

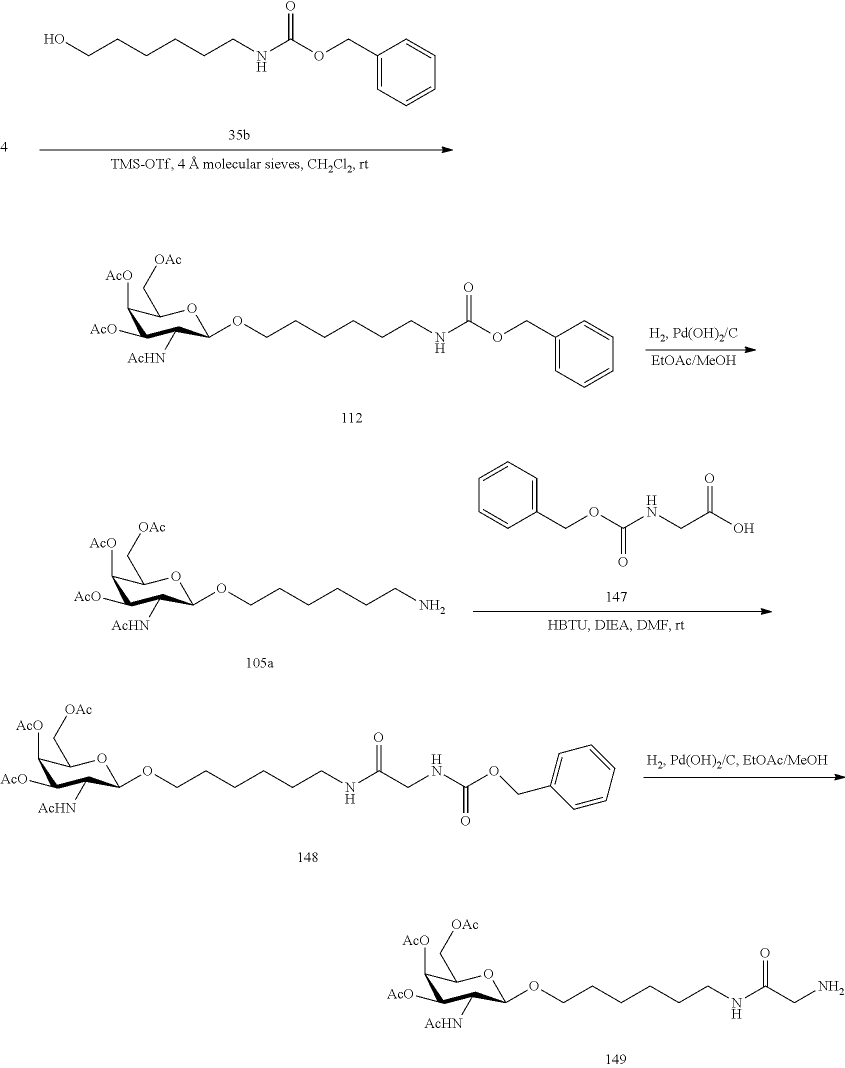

Compound 4 (15 g, 45.55 mmol) and compound 35b (14.3 grams, 57 mmol) were dissolved in CH2C12 (200 ml). Activated molecular sieves (4 A. 2 g, powdered) were added, and the reaction was allowed to stir for 30 minutes under nitrogen atmosphere. TMS-OTf was added (4.1 ml, 22.77 mmol) and the reaction was allowed to stir at room temp overnight. Upon completion, the reaction was quenched by pouring into solution of saturated aqueous NaHCO₃ (500 ml) and crushed ice (−150 g). The organic layer was separated, washed with brine, dried over MgSO₄, filtered, and was concentrated to an orange oil under reduced pressure. The crude material was purified by silica gel column chromatography and eluted with 2-10% MeOH in CH2C12 to yield Compound 112 (16.53 g, 63%). LCMS and ¹H NMR were consistent with the expected compound.

Compound 112 (4.27 g, 7.35 mmol) was dissolved in 1:1 MeOH/EtOAc (40 ml). The reaction mixture was purged by bubbling a stream of argon through the solution for 15 minutes. Pearlman's catalyst (palladium hydroxide on carbon, 400 mg) was added, and hydrogen gas was bubbled through the solution for 30 minutes. Upon completion (TLC 10% MeOH in CH2C12, and LCMS), the catalyst was removed by filtration through a pad of celite. The filtrate was concentrated by rotary evaporation, and was dried briefly under high vacuum to yield Compound 105a (3.28 g). LCMS and 1H NMR were consistent with desired product.

Compound 147 (2.31 g, 11 mmol) was dissolved in anhydrous DMF (100 mL). N,N-Diisopropylethylamine (DIEA, 3.9 mL, 22 mmol) was added, followed by HBTU (4 g, 10.5 mmol). The reaction mixture was allowed to stir for ˜15 minutes under nitrogen. To this a solution of compound 105a (3.3 g, 7.4 mmol) in dry DMF was added and stirred for 2 h under nitrogen atmosphere. The reaction was diluted with EtOAc and washed with saturated aqueous NaHCO₃ and brine. The organics phase was separated, dried (MgSO₄), filtered, and concentrated to an orange syrup. The crude material was purified by column chromatography 2-5% MeOH in CH₂Cl₂ to yield Compound 148 (3.44 g, 73%). LCMS and ¹H NMR were consistent with the expected product.

Compound 148 (3.3 g, 5.2 mmol) was dissolved in 1:1 MeOH/EtOAc (75 ml). The reaction mixture was purged by bubbling a stream of argon through the solution for 15 minutes. Pearlman's catalyst (palladium hydroxide on carbon) was added (350 mg). Hydrogen gas was bubbled through the solution for 30 minutes. Upon completion (TLC 10% MeOH in DCM, and LCMS), the catalyst was removed by filtration through a pad of celite. The filtrate was concentrated by rotary evaporation, and was dried briefly under high vacuum to yield Compound 149 (2.6 g). LCMS was consistent with desired product. The residue was dissolved in dry DMF (10 ml) was used immediately in the next step.

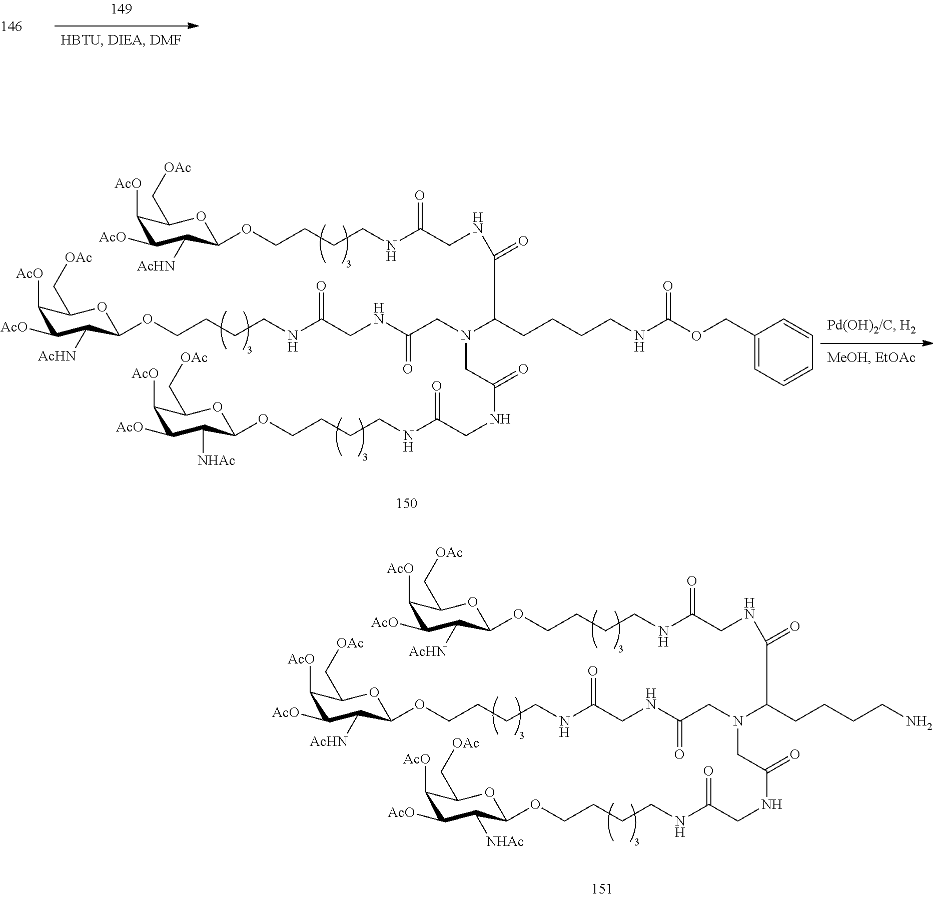

Compound 146 (0.68 g, 1.73 mmol) was dissolved in dry DMF (20 ml). To this DIEA (450 μL, 2.6 mmol, 1.5 eq.) and HBTU (1.96 g, 0.5.2 mmol) were added. The reaction mixture was allowed to stir for 15 minutes at room temperature under nitrogen. A solution of compound 149 (2.6 g) in anhydrous DMF (10 mL) was added. The pH of the reaction was adjusted to pH=9-10 by addition of DIEA (if necessary). The reaction was allowed to stir at room temperature under nitrogen for 2 h. Upon completion the reaction was diluted with EtOAc (100 mL), and washed with aqueous saturated aqueous NaHCO₃, followed by brine. The organic phase was separated, dried over MgSO₄, filtered, and concentrated. The residue was purified by silica gel column chromatography and eluted with 2-10% MeOH in CH₂C2 to yield Compound 150 (0.62 g, 20%). LCMS and ¹H NMR were consistent with the desired product.

Compound 150 (0.62 g) was dissolved in 1:1 MeOH/EtOAc (5 L). The reaction mixture was purged by bubbling a stream of argon through the solution for 15 minutes. Pearlman's catalyst (palladium hydroxide on carbon) was added (60 mg). Hydrogen gas was bubbled through the solution for 30 minutes. Upon completion (TLC 10% MeOH in DCM, and LCMS), the catalyst was removed by filtration (syringe-tip Teflon filter, 0.45 μm). The filtrate was concentrated by rotary evaporation, and was dried briefly under high vacuum to yield Compound 151 (0.57 g). The LCMS was consistent with the desired product. The product was dissolved in 4 mL dry DMF and was used immediately in the next step.

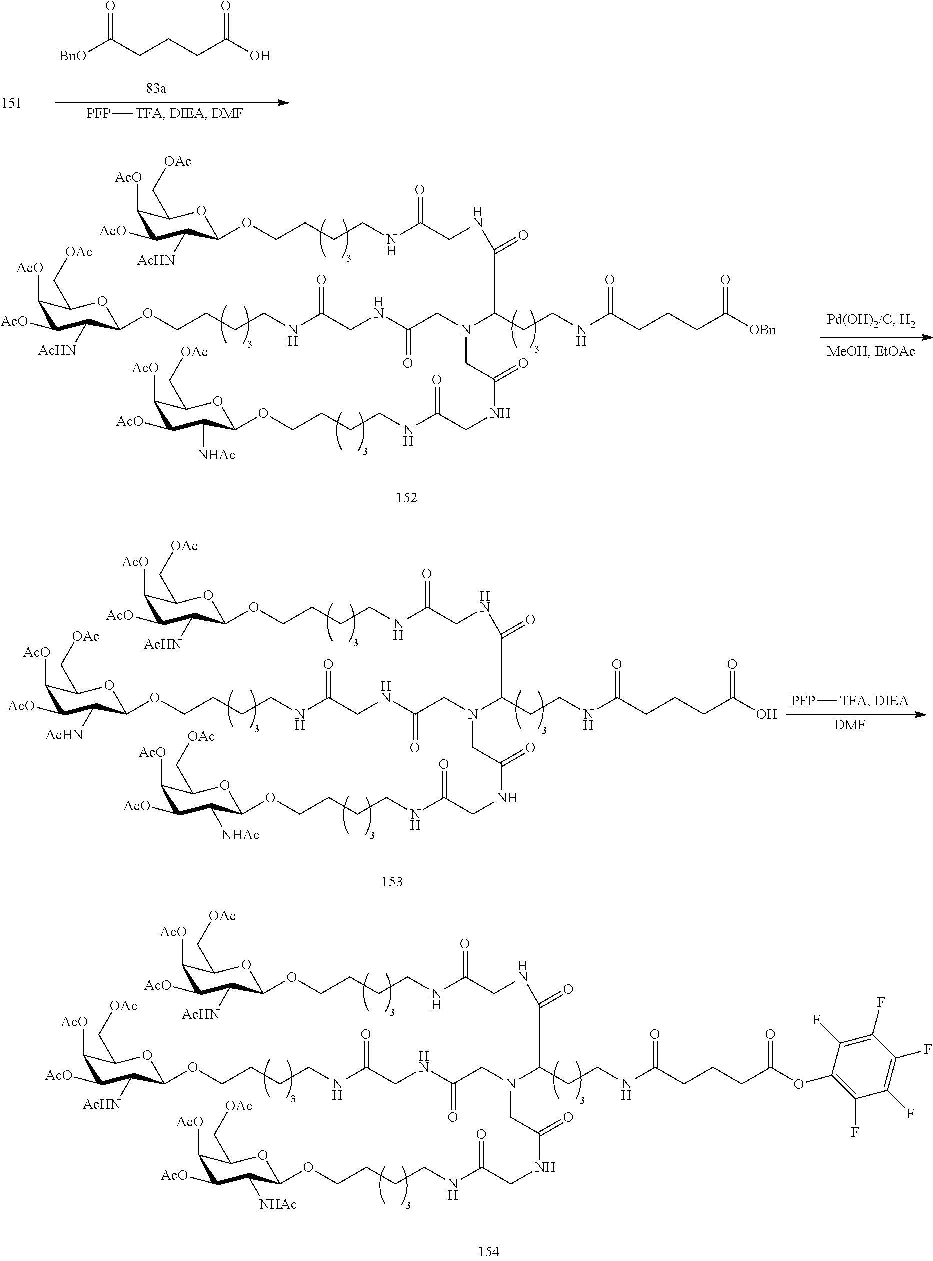

Compound 83a (0.11 g, 0.33 mmol) was dissolved in anhydrous DMF (5 mL) and N,N-Diisopropylethylamine (75 μL, 1 mmol) and PFP-TFA (90 μL, 0.76 mmol) were added. The reaction mixture turned magenta upon contact, and gradually turned orange over the next 30 minutes. Progress of reaction was monitored by TLC and LCMS. Upon completion (formation of the PFP ester), a solution of compound 151 (0.57 g, 0.33 mmol) in DMF was added. The pH of the reaction was adjusted to pH=9-10 by addition of N,N-Diisopropylethylamine (if necessary). The reaction mixture was stirred under nitrogen for ˜30 min. Upon completion, the majority of the solvent was removed under reduced pressure. The residue was diluted with CH₂C2 and washed with aqueous saturated NaHCO₃, followed by brine. The organic phase separated, dried over MgSO₄, filtered, and concentrated to an orange syrup. The residue was purified by silica gel column chromatography (2-10% MeOH in CH₂Cl₂) to yield Compound 152 (0.35 g, 55%). LCMS and ¹H NMR were consistent with the desired product.

Compound 152 (0.35 g, 0.182 mmol) was dissolved in 1:1 MeOH/EtOAc (10 mL). The reaction mixture was purged by bubbling a stream of argon thru the solution for 15 minutes. Pearlman's catalyst (palladium hydroxide on carbon) was added (35 mg). Hydrogen gas was bubbled thru the solution for 30 minutes. Upon completion (TLC 10% MeOH in DCM, and LCMS), the catalyst was removed by filtration (syringe-tip Teflon filter, 0.45 μm). The filtrate was concentrated by rotary evaporation, and was dried briefly under high vacuum to yield Compound 153 (0.33 g, quantitative). The LCMS was consistent with desired product.

Compound 153 (0.33 g, 0.18 mmol) was dissolved in anhydrous DMF (5 mL) with stirring under nitrogen. To this N,N-Diisopropylethylamine (65 μL, 0.37 mmol) and PFP-TFA (35 μL, 0.28 mmol) were added. The reaction mixture was stirred under nitrogen for ˜30 min. The reaction mixture turned magenta upon contact, and gradually turned orange. The pH of the reaction mixture was maintained at pH=9-10 by adding more N,-Diisopropylethylamine. The progress of the reaction was monitored by TLC and LCMS. Upon completion, the majority of the solvent was removed under reduced pressure. The residue was diluted with CH₂Cl₂ (50 mL), and washed with saturated aqueous NaHCO₃, followed by brine. The organic layer was dried over MgSO₄, filtered, and concentrated to an orange syrup. The residue was purified by column chromatography and eluted with 2-10% MeOH in CH₂Cl₂ to yield Compound 154 (0.29 g, 79%). LCMS and ¹H NMR were consistent with the desired product.

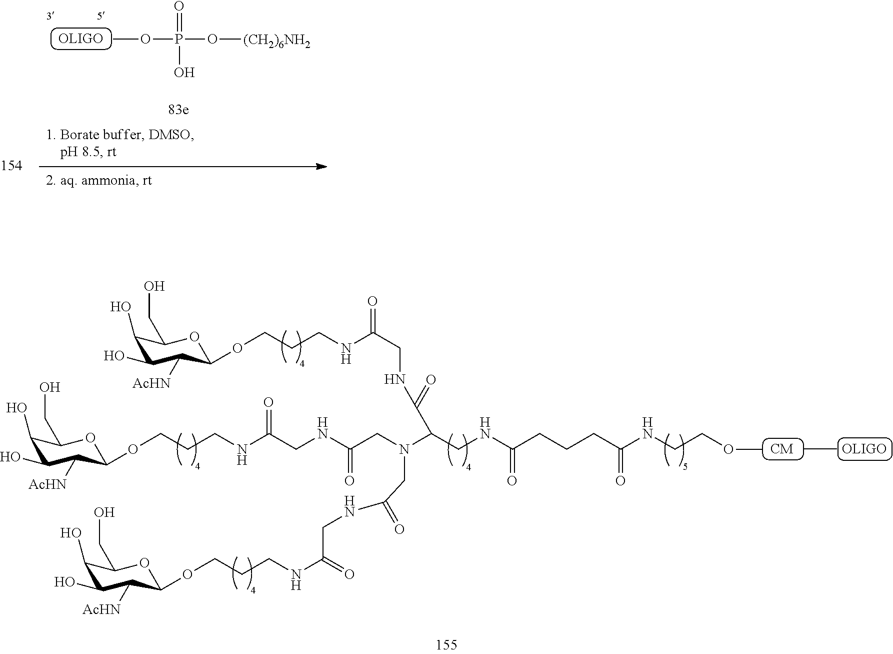

Oligomeric Compound 155, comprising a GalNAc₃-6 conjugate group, was prepared using the general procedures illustrated in Example 46. The GalNAc3 cluster portion of the conjugate group GalNAc₃-6 (GalNAc₃-6ₐ) can be combined with any cleavable moiety to provide a variety of conjugate groups. In certain embodiments, the cleavable moiety is —P(═O)(OH)-A_{d}-P(═O)(OH)—.

The structure of GalNAc₃-6 (GalNAc₃-6ₐ-CM-) is shown below:

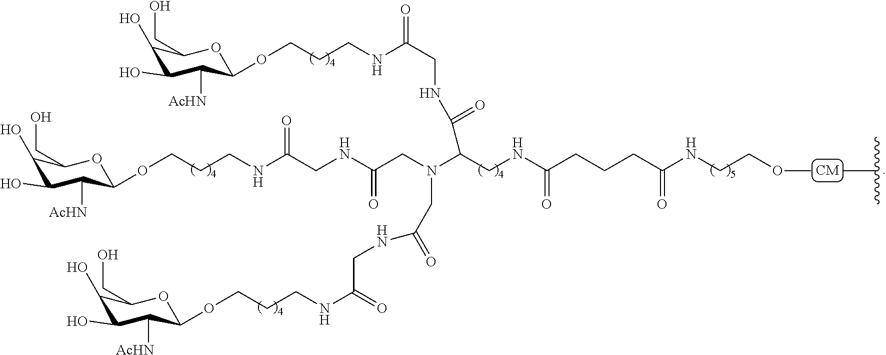

### Example 52: Preparation of Oligonucleotide 160 Comprising GalNAc₃-9

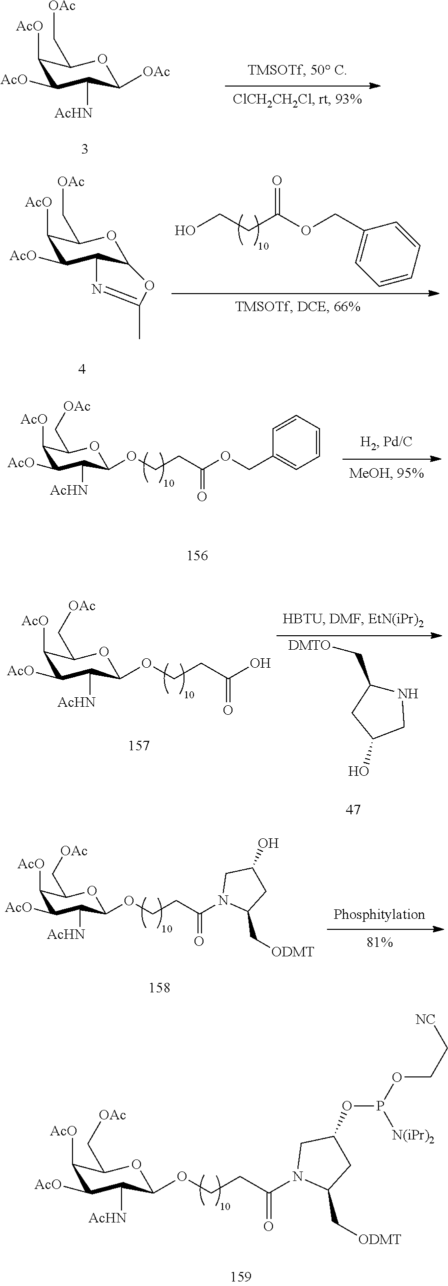

Compound 156 was synthesized following the procedure described in the literature (J. Med. Chem. 2004, 47, 5798-5808).

Compound 156, (18.60 g, 29.28 mmol) was dissolved in methanol (200 mL). Palladium on carbon (6.15 g, 10 wt %, loading (dry basis), matrix carbon powder, wet) was added. The reaction mixture was stirred at room temperature under hydrogen for 18 h. The reaction mixture was filtered through a pad of celite and the celite pad was washed thoroughly with methanol. The combined filtrate was washed and concentrated to dryness. The residue was purified by silica gel column chromatography and eluted with 5-10% methanol in dichloromethane to yield Compound 157 (14.26 g, 89%). Mass m/z 544.1 [M−H]⁻.

Compound 157 (5 g, 9.17 mmol) was dissolved in anhydrous DMF (30 mL). HBTU (3.65 g, 9.61 mmol) and N,N-Diisopropylethylamine (13.73 mL, 78.81 mmol) were added and the reaction mixture was stirred at room temperature for 5 minutes. To this a solution of compound 47 (2.96 g, 7.04 mmol) was added. The reaction was stirred at room temperature for 8 h. The reaction mixture was poured into a saturated NaHCO₃ aqueous solution. The mixture was extracted with ethyl acetate and the organic layer was washed with brine and dried (Na₂SO₄), filtered and evaporated. The residue obtained was purified by silica gel column chromatography and eluted with 50% ethyl acetate in hexane to yield compound 158 (8.25 g, 73.3%). The structure was confirmed by MS and ¹H NMR analysis.

Compound 158 (7.2 g, 7.61 mmol) was dried over P₂O₅ under reduced pressure. The dried compound was dissolved in anhydrous DMF (50 mL). To this 1H-tetrazole (0.43 g, 6.09 mmol) and N-methylimidazole (0.3 mL, 3.81 mmol) and 2-cyanoethyl-N,N,N′,N′-tetraisopropyl phosphorodiamidite (3.65 mL, 11.50 mmol) were added. The reaction mixture was stirred t under an argon atmosphere for 4 h. The reaction mixture was diluted with ethyl acetate (200 mL). The reaction mixture was washed with saturated NaHCO₃ and brine. The organic phase was separated, dried (Na₂SO₄), filtered and evaporated. The residue was purified by silica gel column chromatography and eluted with 50-90% ethyl acetate in hexane to yield Compound 159 (7.82 g, 80.5%). The structure was confirmed by LCMS and ³¹P NMR analysis.

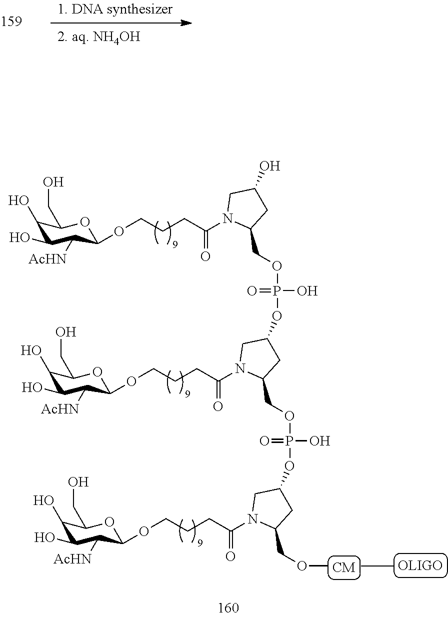

Oligomeric Compound 160, comprising a GalNAc₃-9 conjugate group, was prepared using standard oligonucleotide synthesis procedures. Three units of compound 159 were coupled to the solid support, followed by nucleotide phosphoramidites. Treatment of the protected oligomeric compound with aqueous ammonia yielded compound 160. The GalNAc3 cluster portion of the conjugate group GalNAc₃-9 (GalNAc₃-9ₐ) can be combined with any cleavable moiety to provide a variety of conjugate groups. In certain embodiments, the cleavable moiety is —P(═O)(OH)-A_{d}-P(═O)(OH)—. The structure of GalNAc₃-9 (GalNAc₃-9ₐ-CM) is shown below:

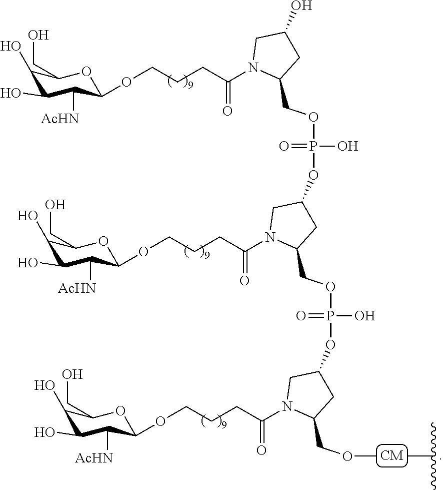

### Example 53: Alternate Procedure for Preparation of Compound 18 (GalNAc₃-1ₐ and GalNAc₃-3ₐ)

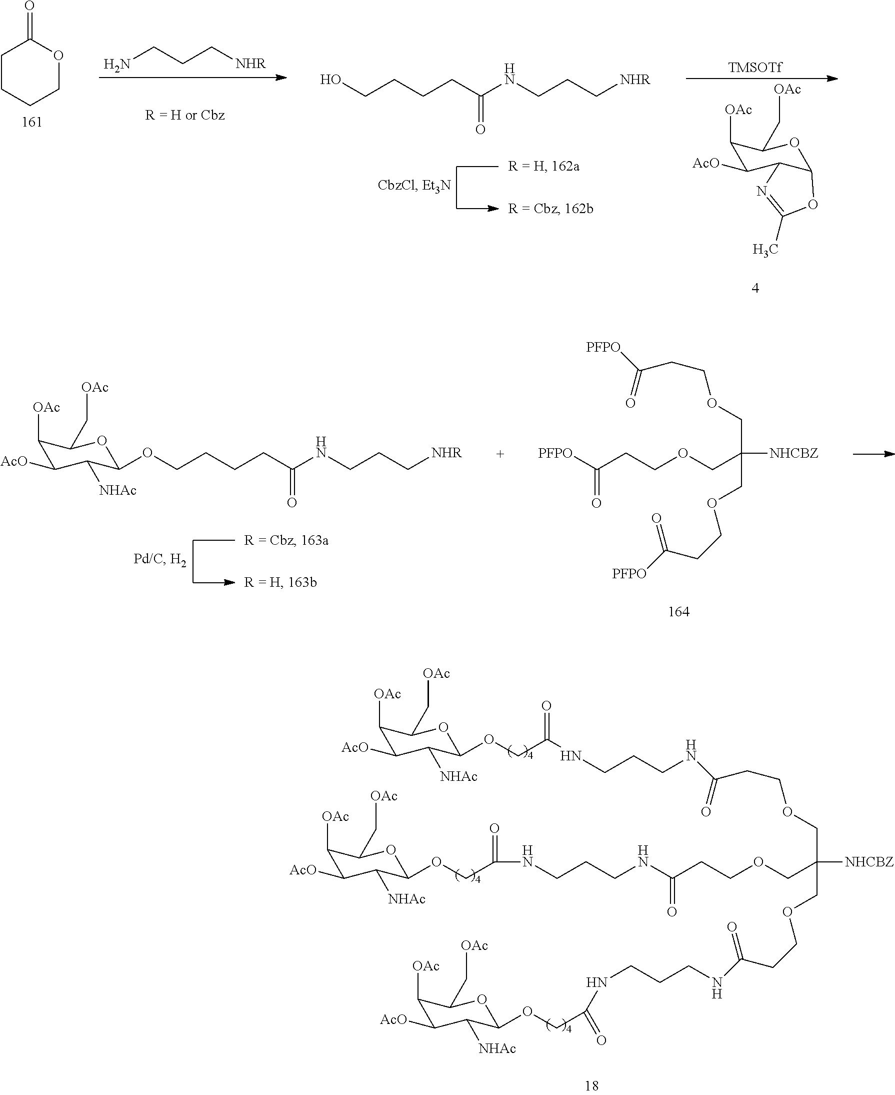

Lactone 161 was reacted with diamino propane (3-5 eq) or Mono-Boc protected diamino propane (1 eq) to provide alcohol 162a or 162b. When unprotected propanediamine was used for the above reaction, the excess diamine was removed by evaporation under high vacuum and the free amino group in 162a was protected using CbzCl to provide 162b as a white solid after purification by column chromatography. Alcohol 162b was further reacted with compound 4 in the presence of TMSOTf to provide 163a which was converted to 163b by removal of the Cbz group using catalytic hydrogenation. The pentafluorophenyl (PFP) ester 164 was prepared by reacting triacid 113 (see Example 48) with PFPTFA (3.5 eq) and pyridine (3.5 eq) in DMF (0.1 to 0.5 M). The triester 164 was directly reacted with the amine 163b (3-4 eq) and DIPEA (3-4 eq) to provide Compound 18. The above method greatly facilitates purification of intermediates and minimizes the formation of byproducts which are formed using the procedure described in Example 4.

### Example 54: Alternate Procedure for Preparation of Compound 18 (GalNAc₃-1ₐ and GalNAc₃-3a)

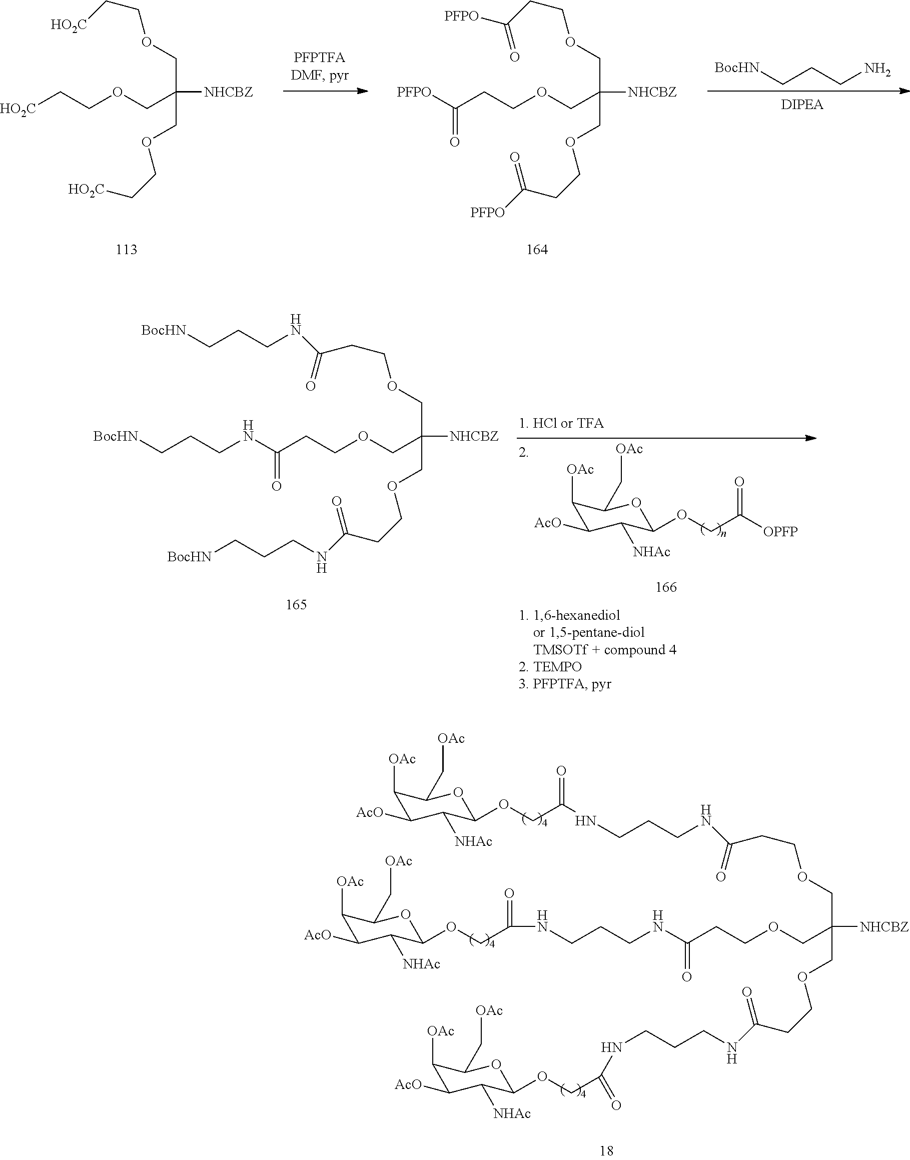

The triPFP ester 164 was prepared from acid 113 using the procedure outlined in example 53 above and reacted with mono-Boc protected diamine to provide 165 in essentially quantitative yield. The Boc groups were removed with hydrochloric acid or trifluoroacetic acid to provide the triamine which was reacted with the PFP activated acid 166 in the presence of a suitable base such as DIPEA to provide Compound 18.

The PFP protected Gal-NAc acid 166 was prepared from the corresponding acid by treatment with PFPTFA (1-1.2 eq) and pyridine (1-1.2 eq) in DMF. The precursor acid in turn was prepared from the corresponding alcohol by oxidation using TEMPO (0.2 eq) and BAIB in acetonitrile and water. The precursor alcohol was prepared from sugar intermediate 4 by reaction with 1,6-hexanediol (or 1,5-pentanediol or other diol for other n values) (2-4 eq) and TMSOTf using conditions described previously in example 47.

### Example 55: Dose-Dependent Study of Oligonucleotides Comprising Either a 3′ or 5′-Conjugate Group (Comparison of GalNAc₃-1, 3, 8 and 9) Targeting SRB-1 In Vivo

The oligonucleotides listed below were tested in a dose-dependent study for antisense inhibition of SRB-1 in mice. Unconjugated ISIS 353382 was included as a standard. Each of the various GalNAc3 conjugate groups was attached at either the 3′ or 5′ terminus of the respective oligonucleotide by a phosphodiester linked 2′-deoxyadenosine nucleoside (cleavable moiety).

TABLE 39
Modified ASO targeting SRB-1

| | | | | SEQ |
|---|---|---|---|---|
| ASO | Sequence (5′ to 3′) | Motif | Conjugate | ID No. |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | none | 829 |
| 353382 | ^{m}C_{ds}T_{ds}Tₑₛ^{m}Cₑₛ^{m}CₑₛTₑₛTₑ | | | |
| (parent) | | | | |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | GalNAc₃-1 | 830 |
| 655861 | ^{m}C_{ds}T_{ds}Tₑₛ^{m}Cₑₛ^{m}CₑₛTₑₛTₑₒA_{do′}-GalNAc₃-1ₐ | | | |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | GalNAc₃-9 | 830 |
| 664078 | ^{m}C_{ds}T_{ds}Tₑₛ^{m}Cₑₛ^{m}CₑₛTₑₛTₑₒA_{do′}-GalNAc₃-9ₐ | | | |
| ISIS | GalNAc₃-3ₐ-_{o′}A_{do} | 5/10/5 | GalNAc₃-3 | 831 |
| 661161 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | | | |
| | ^{m}C_{ds}T_{ds}Tₑₛ^{m}Cₑₛ^{m}CₑₛTₑₛTₑ | | | |
| ISIS | GalNAc₃-8ₐ-_{o′}A_{do} | 5/10/5 | GalNAc₃-8 | 831 |
| 665001 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | | | |
| | ^{m}C_{ds}T_{ds}Tₑₛ^{m}Cₑₛ^{m}CₑₛTₑₛTₑ | | | |

Capital letters indicate the nucleobase for each nucleoside and ^{m}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-1ₐ was shown previously in Example 9. The structure of GalNAc3-9 was shown previously in Example 52. The structure of GalNAc₃-3 was shown previously in Example 39. The structure of GalNAc₃-8 was shown previously in Example 47.

### Treatment

Six week old male Balb/c mice (Jackson Laboratory, Bar Harbor, ME) were injected subcutaneously once at the dosage shown below with ISIS 353382, 655861, 664078, 661161, 665001 or with saline. Each treatment group consisted of 4 animals. The mice were sacrificed 72 hours following the final administration to determine the liver SRB-1 mRNA levels using real-time PCR and RIBOGREEN® RNA quantification reagent (Molecular Probes, Inc. Eugene, OR) according to standard protocols. The results below are presented as the average percent of SRB-1 mRNA levels for each treatment group, normalized to the saline control.

As illustrated in Table 40, treatment with antisense oligonucleotides lowered SRB-1 mRNA levels in a dose-dependent manner. Indeed, the antisense oligonucleotides comprising the phosphodiester linked GalNAc₃-1 and GalNAc₃-9 conjugates at the 3′ terminus (ISIS 655861 and ISIS 664078) and the GalNAc₃-3 and GalNAc₃-8 conjugates linked at the 5′ terminus (ISIS 661161 and ISIS 665001) showed substantial improvement in potency compared to the unconjugated antisense oligonucleotide (ISIS 353382). Furthermore, ISIS 664078, comprising a GalNAc₃-9 conjugate at the 3′ terminus was essentially equipotent compared to ISIS 655861, which comprises a GalNAc₃-1 conjugate at the 3′ terminus. The 5′ conjugated antisense oligonucleotides, ISIS 661161 and ISIS 665001, comprising a GalNAc₃-3 or GalNAc₃-9, respectively, had increased potency compared to the 3′ conjugated antisense oligonucleotides (ISIS 655861 and ISIS 664078).

TABLE 40
ASOs containing GalNAc₃-1, 3, 8 or 9 targeting SRB-1

| ISIS | Dosage | SRB-1 mRNA | |
|---|---|---|---|
| No. | (mg/kg) | (% Saline) | Conjugate |
| Saline | n/a | 100 | |
| 353382 | 3 | 88 | none |
| | 10 | 68 | |
| | 30 | 36 | |
| 655861 | 0.5 | 98 | GalNac3-1 (3′) |
| | 1.5 | 76 | |
| | 5 | 31 | |
| | 15 | 20 | |
| 664078 | 0.5 | 88 | GalNac3-9 (3′) |
| | 1.5 | 85 | |
| | 5 | 46 | |
| | 15 | 20 | |
| 661161 | 0.5 | 92 | GalNac3-3 (5′) |
| | 1.5 | 59 | |
| | 5 | 19 | |
| | 15 | 11 | |
| 665001 | 0.5 | 100 | GalNac3-8 (5′) |
| | 1.5 | 73 | |
| | 5 | 29 | |
| | 15 | 13 | |

Liver transaminase levels, alanine aminotransferase (ALT) and aspartate aminotransferase (AST), in serum were measured relative to saline injected mice using standard protocols. Total bilirubin and BUN were also evaluated. The change in body weights was evaluated with no significant change from the saline group. ALTs, ASTs, total bilirubin and BUN values are shown in the table below.

TABLE 41

| ISIS | Dosage | | | | Total | | |
|---|---|---|---|---|---|---|---|
| No. | mg/kg | | ALT | AST | Bilirubin | BUN | Conjugate |
| Saline | | | 24 | 59 | 0.1 | 37.52 | |
| 353382 | 3 | | 21 | 66 | 0.2 | 34.65 | none |
| | 10 | | 22 | 54 | 0.2 | 34.2 | |
| | 30 | | 22 | 49 | 0.2 | 33.72 | |
| 655861 | 0.5 | | 25 | 62 | 0.2 | 30.65 | GalNac3-1 (3′) |
| | 1.5 | | 23 | 48 | 0.2 | 30.97 | |
| | 5 | | 28 | 49 | 0.1 | 32.92 | |
| | 15 | | 40 | 97 | 0.1 | 31.62 | |
| 664078 | 0.5 | | 40 | 74 | 0.1 | 35.3 | GalNac3-9 (3′) |
| | 1.5 | | 47 | 104 | 0.1 | 32.75 | |
| | 5 | | 20 | 43 | 0.1 | 30.62 | |
| | 15 | | 38 | 92 | 0.1 | 26.2 | |
| 661161 | 0.5 | | 101 | 162 | 0.1 | 34.17 | GalNac3-3 (5′) |
| | 1.5 | g | 42 | 100 | 0.1 | 33.37 | |
| | 5 | g | 23 | 99 | 0.1 | 34.97 | |

| | | | | | | |
|---|---|---|---|---|---|---|
| | 15 | 53 | 83 | 0.1 | 34.8 | |
| 665001 | 0.5 | 28 | 54 | 0.1 | 31.32 | GalNac3-8 (5′) |
| | 1.5 | 42 | 75 | 0.1 | 32.32 | |
| | 5 | 24 | 42 | 0.1 | 31.85 | |
| | 15 | 32 | 67 | 0.1 | 31. | |

### Example 56: Dose-Dependent Study of Oligonucleotides Comprising Either a 3′ or 5′-Conjugate Group (Comparison of GalNAc₃-1, 2, 3, 5, 6, 7 and 10) Targeting SRB-1 In Vivo

The oligonucleotides listed below were tested in a dose-dependent study for antisense inhibition of SRB-1 in mice. Unconjugated ISIS 353382 was included as a standard. Each of the various GalNAc₃ conjugate groups was attached at the 5′ terminus of the respective oligonucleotide by a phosphodiester linked 2′-deoxyadenosine nucleoside (cleavable moiety) except for ISIS 655861 which had the GalNAc₃ conjugate group attached at the 3′ terminus.

TABLE 42
Modified ASO targeting SRB-1

| | | | | SEQ |
|---|---|---|---|---|
| ASO | Sequence (5′ to 3′) | Motif | Conjugate | ID No. |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | no conjugate | 829 |
| 353382 | ^{m}C_{ds}T_{ds}Tₑₛ^{m}Cₑₛ^{m}CₑₛTₑₛTₑ | | | |
| (parent) | | | | |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | GalNAc₃-1 | 830 |
| 655861 | ^{m}C_{ds}T_{ds}Tₑₛ^{m}Cₑₛ^{m}CₑₛTₑₛTₑₒA_{do′}-GalNAc₃-1ₐ | | | |
| ISIS | GalNAc₃-2ₐ-_{o′} | 5/10/5 | GalNAc₃-2 | 831 |
| 664507 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-3ₐ-_{o′}A_{do} | 5/10/5 | GalNAc₃-3 | 831 |
| 661161 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | | | |
| | ^{m}C_{ds}T_{ds}Tₑₛ^{m}Cₑₛ^{m}CₑₛTₑₛTₑ | | | |
| ISIS | GalNAc₃-5ₐ-_{o′} | 5/10/5 | GalNAc₃-5 | 831 |
| 666224 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-6ₐ-_{o′} | 5/10/5 | GalNAc₃-6 | 831 |
| 666961 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-7ₐ-_{o′} | 5/10/5 | GalNAc₃-7 | 831 |
| 666981 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-10ₐ-_{o′} | 5/10/5 | GalNAc₃-10 | 831 |
| 666881 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |

Capital letters indicate the nucleobase for each nucleoside and ^{m}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-1ₐ was shown previously in Example 9. The structure of GalNAc₃-2ₐ was shown previously in Example 37. The structure of GalNAc₃-3ₐ was shown previously in Example 39. The structure of GalNAc₃-5ₐ was shown previously in Example 49. The structure of GalNAc₃-6ₐ was shown previously in Example 51. The structure of GalNAc₃-7ₐ was shown previously in Example 48. The structure of GalNAc₃-10ₐ was shown previously in Example 46.

### Treatment

Six week old male Balb/c mice (Jackson Laboratory, Bar Harbor, ME) were injected subcutaneously once at the dosage shown below with ISIS 353382, 655861, 664507, 661161, 666224, 666961, 666981, 666881 or with saline. Each treatment group consisted of 4 animals. The mice were sacrificed 72 hours following the final administration to determine the liver SRB-1 mRNA levels using real-time PCR and RIBOGREEN® RNA quantification reagent (Molecular Probes, Inc. Eugene, OR) according to standard protocols. The results below are presented as the average percent of SRB-1 mRNA levels for each treatment group, normalized to the saline control.

As illustrated in Table 43, treatment with antisense oligonucleotides lowered SRB-1 mRNA levels in a dose-dependent manner. Indeed, the conjugated antisense oligonucleotides showed substantial improvement in potency compared to the unconjugated antisense oligonucleotide (ISIS 353382). The 5′ conjugated antisense oligonucleotides showed a slight increase in potency compared to the 3′ conjugated antisense oligonucleotide.

TABLE 43

| ISIS | Dosage | SRB-1 mRNA | |
|---|---|---|---|
| No. | (mg/kg) | (% Saline) | Conjugate |
| Saline | n/a | 100.0 | |
| 353382 | 3 | 96.0 | none |
| | 10 | 73.1 | |
| | 30 | 36.1 | |
| 655861 | 0.5 | 99.4 | GalNac3-1 (3′) |
| | 1.5 | 81.2 | |
| | 5 | 33.9 | |
| | 15 | 15.2 | |
| 664507 | 0.5 | 102.0 | GalNac3-2 (5′) |
| | 1.5 | 73.2 | |
| | 5 | 31.3 | |
| | 15 | 10.8 | |
| 661161 | 0.5 | 90.7 | GalNac3-3 (5′) |
| | 1.5 | 67.6 | |
| | 5 | 24.3 | |
| | 15 | 11.5 | |
| 666224 | 0.5 | 96.1 | GalNac3-5 (5′) |
| | 1.5 | 61.6 | |
| | 5 | 25.6 | |
| | 15 | 11.7 | |
| 666961 | 0.5 | 85.5 | GalNAc3-6 (5′) |
| | 1.5 | 56.3 | |
| | 5 | 34.2 | |
| | 15 | 13.1 | |
| 666981 | 0.5 | 84.7 | GalNAc3-7 (5′) |
| | 1.5 | 59.9 | |
| | 5 | 24.9 | |
| | 15 | 8.5 | |
| 666881 | 0.5 | 100.0 | GalNAc3-10 (5′) |
| | 1.5 | 65.8 | |
| | 5 | 26.0 | |
| | 15 | 13.0 | |

Liver transaminase levels, alanine aminotransferase (ALT) and aspartate aminotransferase (AST), in serum were measured relative to saline injected mice using standard protocols. Total bilirubin and BUN were also evaluated. The change in body weights was evaluated with no significant change from the saline group. ALTs, ASTs, total bilirubin and BUN values are shown in Table 44 below.

TABLE 44

| ISIS | Dosage | | | | Total | | |
|---|---|---|---|---|---|---|---|
| No. | mg/kg | | ALT | AST | Bilirubin | BUN | Conjugate |
| Saline | | | 26 | 57 | 0.2 | 27 | |
| 353382 | 3 | | 25 | 92 | 0.2 | 27 | none |
| | 10 | | 23 | 40 | 0.2 | 25 | |
| | 30 | | 29 | 54 | 0.1 | 28 | |
| 655861 | 0.5 | | 25 | 71 | 0.2 | 34 | GalNac3-1 (3′) |
| | 1.5 | | 28 | 60 | 0.2 | 26 | |
| | 5 | | 26 | 63 | 0.2 | 28 | |
| | 15 | | 25 | 61 | 0.2 | 28 | |
| 664507 | 0.5 | | 25 | 62 | 0.2 | 25 | GalNac3-2 (5′) |
| | 1.5 | | 24 | 49 | 0.2 | 26 | |
| | 5 | | 21 | 50 | 0.2 | 26 | |
| | 15 | | 59 | 84 | 0.1 | 22 | |
| 661161 | 0.5 | | 20 | 42 | 0.2 | 29 | GalNac3-3 (5′) |
| | 1.5 | g | 37 | 74 | 0.2 | 25 | |
| | 5 | g | 28 | 61 | 0.2 | 29 | |

| | | | | | | |
|---|---|---|---|---|---|---|
| | 15 | 21 | 41 | 0.2 | 25 | |
| 666224 | 0.5 | 34 | 48 | 0.2 | 21 | GalNac3-5 (5′) |
| | 1.5 | 23 | 46 | 0.2 | 26 | |
| | 5 | 24 | 47 | 0.2 | 23 | |
| | 15 | 32 | 49 | 0.1 | 26 | |
| 666961 | 0.5 | 17 | 63 | 0.2 | 26 | GalNAc3-6 (5′) |
| | 1.5 | 23 | 68 | 0.2 | 26 | |
| | 5 | 25 | 66 | 0.2 | 26 | |
| | 15 | 29 | 107 | 0.2 | 28 | |
| 666981 | 0.5 | 24 | 48 | 0.2 | 26 | GalNAc3-7 (5′) |
| | 1.5 | 30 | 55 | 0.2 | 24 | |
| | 5 | 46 | 74 | 0.1 | 24 | |
| | 15 | 29 | 58 | 0.1 | 26 | |
| 666881 | 0.5 | 20 | 65 | 0.2 | 27 | GalNAc3-10 (5′) |
| | 1.5 | 23 | 59 | 0.2 | 24 | |
| | 5 | 45 | 70 | 0.2 | 26 | |
| | 15 | 21 | 57 | 0.2 | 24 | |

### Example 57: Duration of Action Study of Oligonucleotides Comprising a 3′-Conjugate Group Targeting ApoC III In Vivo

Mice were injected once with the doses indicated below and monitored over the course of 42 days for ApoC-III and plasma triglycerides (Plasma TG) levels. The study was performed using 3 transgenic mice that express human APOC-II in each group.

TABLE 45
Modified ASO targeting ApoC III

| | | | SEQ |
|---|---|---|---|
| | | | ID |
| ASO | Sequence (5′ to 3′) | Linkages | No. |
| ISIS | AₑₛGₑₛ^{m}CₑₛTₑₛTₑₛ^{m}C_{ds}T_{ds}T_{ds}G_{ds}T_{ds} | PS | 821 |
| 304801 | ^{m}C_{ds}^{m}C_{ds}A_{ds}G_{ds}^{m}C_{ds}TₑₛTₑₛTₑₛAₑₛTₑ | | |
| ISIS | AₑₛGₑₛ^{m}CₑₛTₑₛTₑₛ^{m}C_{ds}T_{ds}T_{ds}G_{ds}T_{ds} | PS | 822 |
| 647535 | ^{m}C_{ds}^{m}C_{ds}A_{ds}G_{ds}^{m}C_{ds}TₑₛTₑₛTₑₛAₑₛTₑₒ | | |
| | A_{do′}-GalNAc₃-1ₐ | | |
| ISIS | AₑₛGₑₒ^{m}CₑₒTₑₒTₑₒ^{m}C_{ds}T_{ds}T_{ds}G_{ds}T_{ds} | PO/PS | 822 |
| 647536 | ^{m}C_{ds}^{m}C_{ds}A_{ds}G_{ds}^{m}C_{ds}TₑₒTₑₒTₑₛAₑₛTₑₒ | | |
| | A_{do′}-GalNAc₃-1ₐ | | |

Capital letters indicate the nucleobase for each nucleoside and ^{tm}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-1ₐ was shown previously in Example 9.

TABLE 46
ApoC III mRNA (% Saline on Day 1) and Plasma TG Levels (% Saline on Day 1)

| ASO | Dose | | Target | Day 3 | Day 7 | Day 14 | Day 35 | Day 42 |
|---|---|---|---|---|---|---|---|---|
| Saline | 0 | mg/kg | ApoC-III | 98 | 100 | 100 | 95 | 116 |
| ISIS 304801 | 30 | mg/kg | ApoC-III | 28 | 30 | 41 | 65 | 74 |
| ISIS 647535 | 10 | mg/kg | ApoC-III | 16 | 19 | 25 | 74 | 94 |
| ISIS 647536 | 10 | mg/kg | ApoC-III | 18 | 16 | 17 | 35 | 51 |
| Saline | 0 | mg/kg | Plasma TG | 121 | 130 | 123 | 105 | 109 |
| ISIS 304801 | 30 | mg/kg | Plasma TG | 34 | 37 | 50 | 69 | 69 |
| ISIS 647535 | 10 | mg/kg | Plasma TG | 18 | 14 | 24 | 18 | 71 |
| ISIS 647536 | 10 | mg/kg | Plasma TG | 21 | 19 | 15 | 32 | 35 |

As can be seen in the table above the duration of action increased with addition of the 3′-conjugate group compared to the unconjugated oligonucleotide. There was a further increase in the duration of action for the conjugated mixed PO/PS oligonucleotide 647536 as compared to the conjugated full PS oligonucleotide 647535.

### Example 58: Dose-Dependent Study of Oligonucleotides Comprising a 3′-Conjugate Group (Comparison of GalNAc₃-1 and GalNAc₄-11) Targeting SRB-1 In Vivo

The oligonucleotides listed below were tested in a dose-dependent study for antisense inhibition of SRB-1 in mice. Unconjugated ISIS 440762 was included as an unconjugated standard. Each of the conjugate groups were attached at the 3′ terminus of the respective oligonucleotide by a phosphodiester linked 2′-deoxyadenosine nucleoside cleavable moiety.

The structure of GalNAc₃-1ₐ was shown previously in Example 9. The structure of GalNAc3-11ₐ was shown previously in Example 50.

### Treatment

Six week old male Balb/c mice (Jackson Laboratory, Bar Harbor, ME) were injected subcutaneously once at the dosage shown below with ISIS 440762, 651900, 663748 or with saline. Each treatment group consisted of 4 animals. The mice were sacrificed 72 hours following the final administration to determine the liver SRB-1 mRNA levels using real-time PCR and RIBOGREEN® RNA quantification reagent (Molecular Probes, Inc. Eugene, OR) according to standard protocols. The results below are presented as the average percent of SRB-1 mRNA levels for each treatment group, normalized to the saline control.

As illustrated in Table 47, treatment with antisense oligonucleotides lowered SRB-1 mRNA levels in a dose-dependent manner. The antisense oligonucleotides comprising the phosphodiester linked GalNAc₃-1 and GalNAc₄-11 conjugates at the 3′ terminus (ISIS 651900 and ISIS 663748) showed substantial improvement in potency compared to the unconjugated antisense oligonucleotide (ISIS 440762). The two conjugated oligonucleotides, GalNAc₃-1 and GalNAc₄-11, were equipotent.

TABLE 47
Modified ASO targeting SRB-1

| | | | % Saline | SEQ ID |
|---|---|---|---|---|
| ASO | Sequence (5′ to 3′) | Dose mg/kg | control | No. |
| Saline | | | 100 | |
| ISIS | Tₖₛ^{m}CₖₛA_{ds}G_{ds}T_{ds}^{m}C_{ds}A_{ds}T_{ds}G_{ds}A_{ds} | 0.6 | 73.45 | 823 |
| 440762 | ^{m}C_{ds}T_{ds}Tₖₛ^{m}Cₖ | 2 | 59.66 | |
| | | 6 | 23.50 | |
| ISIS | Tₖₛ^{m}CₖₛA_{ds}G_{ds}T_{ds}^{m}C_{ds}A_{ds}T_{ds}G_{ds}A_{ds} | 0.2 | 62.75 | 824 |
| 651900 | ^{m}C_{ds}T_{ds}Tₖₛ^{m}CₖₒA_{do′}-GalNAc₃-1ₐ | 0.6 | 29.14 | |
| | | 2 | 8.61 | |
| | | 6 | 5.62 | |
| ISIS | Tₖₛ^{m}CₖₛA_{ds}G_{ds}T_{ds}^{m}C_{ds}A_{ds}T_{ds}G_{ds}A_{ds} | 0.2 | 63.99 | 824 |
| 663748 | ^{m}C_{ds}T_{ds}Tₖₛ^{m}CₖₒA_{do′}-GalNAc₄-11ₐ | 0.6 | 33.53 | |
| | | 2 | 7.58 | |
| | | 6 | 5.52 | |

Capital letters indicate the nucleobase for each nucleoside and ^{m}C indicates a 5-methyl cytosine. Subscripts: “e” indicates a 2′-MOE modified nucleoside; “k” indicates 6′-(S)—CH₃ bicyclic nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

Liver transaminase levels, alanine aminotransferase (ALT) and aspartate aminotransferase (AST), in serum were measured relative to saline injected mice using standard protocols. Total bilirubin and BUN were also evaluated. The change in body weights was evaluated with no significant change from the saline group. ALTs, ASTs, total bilirubin and BUN values are shown in Table 48 below.

TABLE 48

| ISIS | Dosage | | | Total | | |
|---|---|---|---|---|---|---|
| No. | mg/kg | ALT | AST | Bilirubin | BUN | Conjugate |
| Saline | | 30 | 76 | 0.2 | 40 | |
| 440762 | 0.60 | 32 | 70 | 0.1 | 35 | none |
| | 2 | 26 | 57 | 0.1 | 35 | |
| | 6 | 31 | 48 | 0.1 | 39 | |
| 651900 | 0.2 | 32 | 115 | 0.2 | 39 | GalNac3-1 (3′) |
| | 0.6 | 33 | 61 | 0.1 | 35 | |
| | 2 | 30 | 50 | 0.1 | 37 | |
| | 6 | 34 | 52 | 0.1 | 36 | |
| 663748 | 0.2 | 28 | 56 | 0.2 | 36 | GalNac4-11 (3′) |
| | 0.6 | 34 | 60 | 0.1 | 35 | |
| | 2 | 44 | 62 | 0.1 | 36 | |
| | 6 | 38 | 71 | 0.1 | 33 | |

### Example 59: Effects of GalNAc₃-1 Conjugated ASOs Targeting FXI In Vivo

The oligonucleotides listed below were tested in a multiple dose study for antisense inhibition of FXI in mice. ISIS 404071 was included as an unconjugated standard. Each of the conjugate groups was attached at the 3′ terminus of the respective oligonucleotide by a phosphodiester linked 2′-deoxyadenosine nucleoside cleavable moiety.

TABLE 49
Modified ASOs targeting FXI

| | | | SEQ |
|---|---|---|---|
| | | | ID |
| ASO | Sequence (5′ to 3′) | Linkages | No. |
| ISIS | TₑₛGₑₛGₑₛTₑₛAₑₛA_{ds}T_{ds}^{m}C_{ds}^{m}C_{ds}A_{ds}^{m}C_{ds} | PS | 832 |
| 404071 | T_{ds}T_{ds}T_{ds}^{m}C_{ds}AₑₛGₑₛAₑₛGₑₛGₑ | | |
| ISIS | TₑₛGₑₛGₑₛTₑₛAₑₛA_{ds}T_{ds}^{m}C_{ds}^{m}C_{ds}A_{ds}^{m}C_{ds} | PS | 833 |
| 656172 | T_{ds}T_{ds}T_{ds}^{m}C_{ds}AₑₛGₑₛAₑₛGₑₛGₑₒA_{do′}- | | |
| | GalNAc₃-1ₐ | | |
| ISIS | TₑₛGₑₒGₑₒTₑₒAₑₒA_{ds}T_{ds}^{m}C_{ds}^{m}C_{ds}A_{ds}^{m}C_{ds} | PO/PS | 833 |
| 656173 | T_{ds}T_{ds}T_{ds}^{m}C_{ds}AₑₒGₑₒAₑₛGₑₛGₑₒA_{do′}- | | |
| | GalNAc₃-1ₐ | | |

Capital letters indicate the nucleobase for each nucleoside and ^{m}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-1ₐ was shown previously in Example 9.

### Treatment

Six week old male Balb/c mice (Jackson Laboratory, Bar Harbor, ME) were injected subcutaneously twice a week for 3 weeks at the dosage shown below with ISIS 404071, 656172, 656173 or with PBS treated control. Each treatment group consisted of 4 animals. The mice were sacrificed 72 hours following the final administration to determine the liver FXI mRNA levels using real-time PCR and RIBOGREEN® RNA quantification reagent (Molecular Probes, Inc. Eugene, OR) according to standard protocols. Plasma FXI protein levels were also measured using ELISA. FXI mRNA levels were determined relative to total RNA (using RIBOGREEN®), prior to normalization to PBS-treated control. The results below are presented as the average percent of FXI mRNA levels for each treatment group. The data was normalized to PBS-treated control and is denoted as “% PBS”.

The ED_{50S} were measured using similar methods as described previously and are presented below.

TABLE 50
Factor XI mRNA (% Saline)

| | Dose | % | | |
|---|---|---|---|---|
| ASO | mg/kg | Control | Conjugate | Linkages |
| Saline | | 100 | none | |
| ISIS | 3 | 92 | none | PS |
| 404071 | 10 | 40 | | |
| | 30 | 15 | | |
| ISIS | 0.7 | 74 | GalNAc3-1 | PS |
| 656172 | 2 | 33 | | |
| | 6 | 9 | | |
| ISIS | 0.7 | 49 | GalNAc3-1 | PO/PS |
| 656173 | 2 | 22 | | |
| | 6 | 1 | | |

As illustrated in Table 50, treatment with antisense oligonucleotides lowered FXI mRNA levels in a dose-dependent manner. The oligonucleotides comprising a 3′-GalNAc₃-1 conjugate group showed substantial improvement in potency compared to the unconjugated antisense oligonucleotide (ISIS 404071). Between the two conjugated oligonucleotides an improvement in potency was further provided by substituting some of the PS linkages with PO (ISIS 656173).

As illustrated in Table 50a, treatment with antisense oligonucleotides lowered FXI protein levels in a dose-dependent manner. The oligonucleotides comprising a 3′-GalNAc₃-1 conjugate group showed substantial improvement in potency compared to the unconjugated antisense oligonucleotide (ISIS 404071). Between the two conjugated oligonucleotides an improvement in potency was further provided by substituting some of the PS linkages with PO (ISIS 656173).

TABLE 50a
Factor XI protein (% Saline)

| | Dose | Protein | | |
|---|---|---|---|---|
| ASO | mg/kg | (% Control) | Conjugate | Linkages |
| Saline | | 100 | none | |
| ISIS | 3 | 127 | none | PS |
| 404071 | 10 | 32 | | |
| | 30 | 3 | | |
| ISIS | 0.7 | 70 | GalNAc3-1 | PS |
| 656172 | 2 | 23 | | |
| | 6 | 1 | | |
| ISIS | 0.7 | 45 | GalNAc3-1 | PO/PS |
| 656173 | 2 | 6 | | |
| | 6 | 0 | | |

Liver transaminase levels, alanine aminotransferase (ALT) and aspartate aminotransferase (AST), in serum were measured relative to saline injected mice using standard protocols. Total bilirubin, total albumin, CRE and BUN were also evaluated. The change in body weights was evaluated with no significant change from the saline group. ALTs, ASTs, total bilirubin and BUN values are shown in the table below.

TABLE 51

| ISIS | Dosage | | | Total | Total | | | |
|---|---|---|---|---|---|---|---|---|
| No. | mg/kg | ALT | AST | Albumin | Bilirubin | CRE | BUN | Conjugate |
| Saline | | 71.8 | 84.0 | 3.1 | 0.2 | 0.2 | 22.9 | |
| 404071 | 3 | 152.8 | 176.0 | 3.1 | 0.3 | 0.2 | 23.0 | none |
| | 10 | 73.3 | 121.5 | 3.0 | 0.2 | 0.2 | 21.4 | |
| | 30 | 82.5 | 92.3 | 3.0 | 0.2 | 0.2 | 23.0 | |
| 656172 | 0.7 | 62.5 | 111.5 | 3.1 | 0.2 | 0.2 | 23.8 | GalNac3-1 (3′) |
| | 2 | 33.0 | 51.8 | 2.9 | 0.2 | 0.2 | 22.0 | |
| | 6 | 65.0 | 71.5 | 3.2 | 0.2 | 0.2 | 23.9 | |
| 656173 | 0.7 | 54.8 | 90.5 | 3.0 | 0.2 | 0.2 | 24.9 | GalNac3-1 (3′) |
| | 2 | 85.8 | 71.5 | 3.2 | 0.2 | 0.2 | 21.0 | |
| | 6 | 114.0 | 101.8 | 3.3 | 0.2 | 0.2 | 22.7 | |

### Example 60: Effects of Conjugated ASOs Targeting SRB-1 in Vitro

The oligonucleotides listed below were tested in a multiple dose study for antisense inhibition of SRB-1 in primary mouse hepatocytes. ISIS 353382 was included as an unconjugated standard. Each of the conjugate groups were attached at the 3′ or 5′ terminus of the respective oligonucleotide by a phosphodiester linked 2-deoxyadenosine nucleoside cleavable moiety.

TABLE 52
Modified ASO targeting SRB-1

| | | | | SEQ |
|---|---|---|---|---|
| | | | | ID |
| ASO | Sequence (5′ to 3′) | Motif | Conjugate | No. |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | none | 829 |
| 353382 | ^{m}C_{ds}T_{ds}Tₑₛ^{m}Cₑₛ^{m}CₑₛTₑₛTₑ | | | |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | GalNAc₃-1 | 830 |
| 655861 | ^{m}C_{ds}T_{ds}Tₑₛ^{m}Cₑₛ^{m}CₑₛTₑₛTₑₒA_{do′}-GalNAc₃-1ₐ | | | |
| ISIS | Gₑₛ^{m}CₑₒTₑₒTₑₒ^{m}CₑₒA_{ds}G_{ds}T_{ds}^{m}C_{ds}A_{ds}T_{ds}G_{ds}A_{ds} | 5/10/5 | GalNAc₃-1 | 830 |
| 655862 | ^{m}C_{ds}T_{ds}Tₑₛ^{m}Cₑₒ^{m}CₑₛTₑₛTₑₒA_{do′}-GalNAc₃-1ₐ | | | |
| ISIS | GalNAc₃-3_{a-o′}A_{do}Gₑₛ^{m}CₑₛTₑₛTₑₛ^{m}CₑₛA_{ds}G_{ds} | 5/10/5 | GalNAc₃-3 | 831 |
| 661161 | T_{ds}^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-8_{a-o′}A_{do}Gₑₛ^{m}CₑₛTₑₛTₑₛ^{m}CₑₛA_{ds}G_{ds} | 5/10/5 | GalNAc₃-8 | 831 |
| 665001 | T_{ds}^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | 5/10/5 | GalNAc₃-9 | 830 |
| 664078 | ^{m}C_{ds}T_{ds}Tₑₛ^{m}Cₑₛ^{m}CₑₛTₑₛTₑₒA_{do′}-GalNAc₃-9ₐ | | | |
| ISIS | GalNAc₃-6ₐ-_{o′}A_{do}Gₑₛ^{m}CₑₛTₑₛTₑₛ^{m}CₑₛA_{ds}G_{ds} | 5/10/5 | GalNAc₃-6 | 831 |
| 666961 | T_{ds}^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-2ₐ-_{o′} | 5/10/5 | GalNAc₃-2 | 831 |
| 664507 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-10ₐ-_{o′} | 5/10/5 | GalNAc₃-10 | 831 |
| 666881 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-5ₐ-_{o′} | 5/10/5 | GalNAc₃-5 | 831 |
| 666224 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| ISIS | GalNAc₃-7ₐ-_{o′} | 5/10/5 | GalNAc₃-7 | 831 |
| 666981 | A_{do}Gₑₛ^{m}CₑₛTₑₛTₑₛ^{m}CₑₛA_{ds}G_{ds}T_{ds} | | | |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |

Capital letters indicate the nucleobase for each nucleoside and ^{tm}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-1ₐ was shown previously in Example 9. The structure of GalNAc3-3a was shown previously in Example 39. The structure of GalNAc₃-8ₐ was shown previously in Example 47. The structure of GalNAc₃-9ₐ was shown previously in Example 52. The structure of GalNAc₃-6ₐ was shown previously in Example 51. The structure of GalNAc₃-2ₐ was shown previously in Example 37. The structure of GalNAc₃-10ₐ was shown previously in Example 46. The structure of GalNAc₃-5ₐ was shown previously in Example 49. The structure of GalNAc₃-7ₐ was shown previously in Example 48.

### Treatment

The oligonucleotides listed above were tested in vitro in primary mouse hepatocyte cells plated at a density of 25,000 cells per well and treated with 0.03, 0.08, 0.24, 0.74, 2.22, 6.67 or 20 nM modified oligonucleotide. After a treatment period of approximately 16 hours, RNA was isolated from the cells and mRNA levels were measured by quantitative real-time PCR and the SRB-1 mRNA levels were adjusted according to total RNA content, as measured by RIBOGREEN®.

The IC₅₀ was calculated using standard methods and the results are presented in Table 53. The results show that, under free uptake conditions in which no reagents or electroporation techniques are used to artificially promote entry of the oligonucleotides into cells, the oligonucleotides comprising a GalNAc conjugate were significantly more potent in hepatocytes than the parent oligonucleotide (ISIS 353382) that does not comprise a GalNAc conjugate.

TABLE 53

| | IC₅₀ | Internucleoside | | SEQ |
|---|---|---|---|---|
| ASO | (nM) | linkages | Conjugate | ID No. |
| ISIS 353382 | 190^{a} | PS | none | 829 |
| ISIS 655861 | 11^{a} | PS | GalNAc3-1 | 830 |
| ISIS 655862 | 3 | PO/PS | GalNAc3-1 | 830 |
| ISIS 661161 | 15^{a} | PS | GalNAc3-3 | 831 |
| ISIS 665001 | 20 | PS | GalNAc3-8 | 831 |
| ISIS 664078 | 55 | PS | GalNAc3-9 | 830 |
| ISIS 666961 | 22^{a} | PS | GalNAc3-6 | 831 |
| ISIS 664507 | 30 | PS | GalNAc3-2 | 831 |
| ISIS 666881 | 30 | PS | GalNAc3-10 | 831 |
| ISIS 666224 | 30^{a} | PS | GalNAc3-5 | 831 |
| ISIS 666981 | 40 | PS | GalNAc3-7 | 831 |

^{a}Average of multiple runs.

### Example 61: Preparation of Oligomeric Compound 175 Comprising GalNAc₃-12

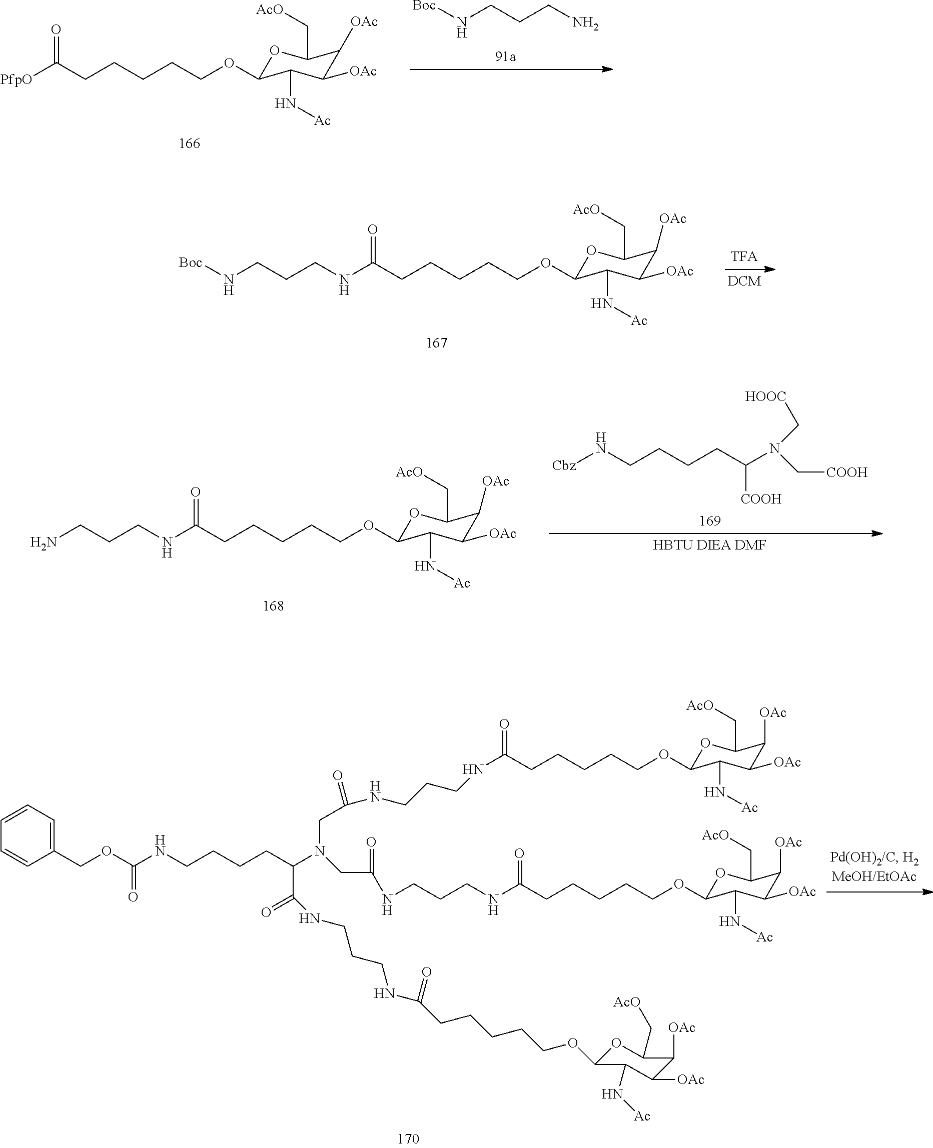

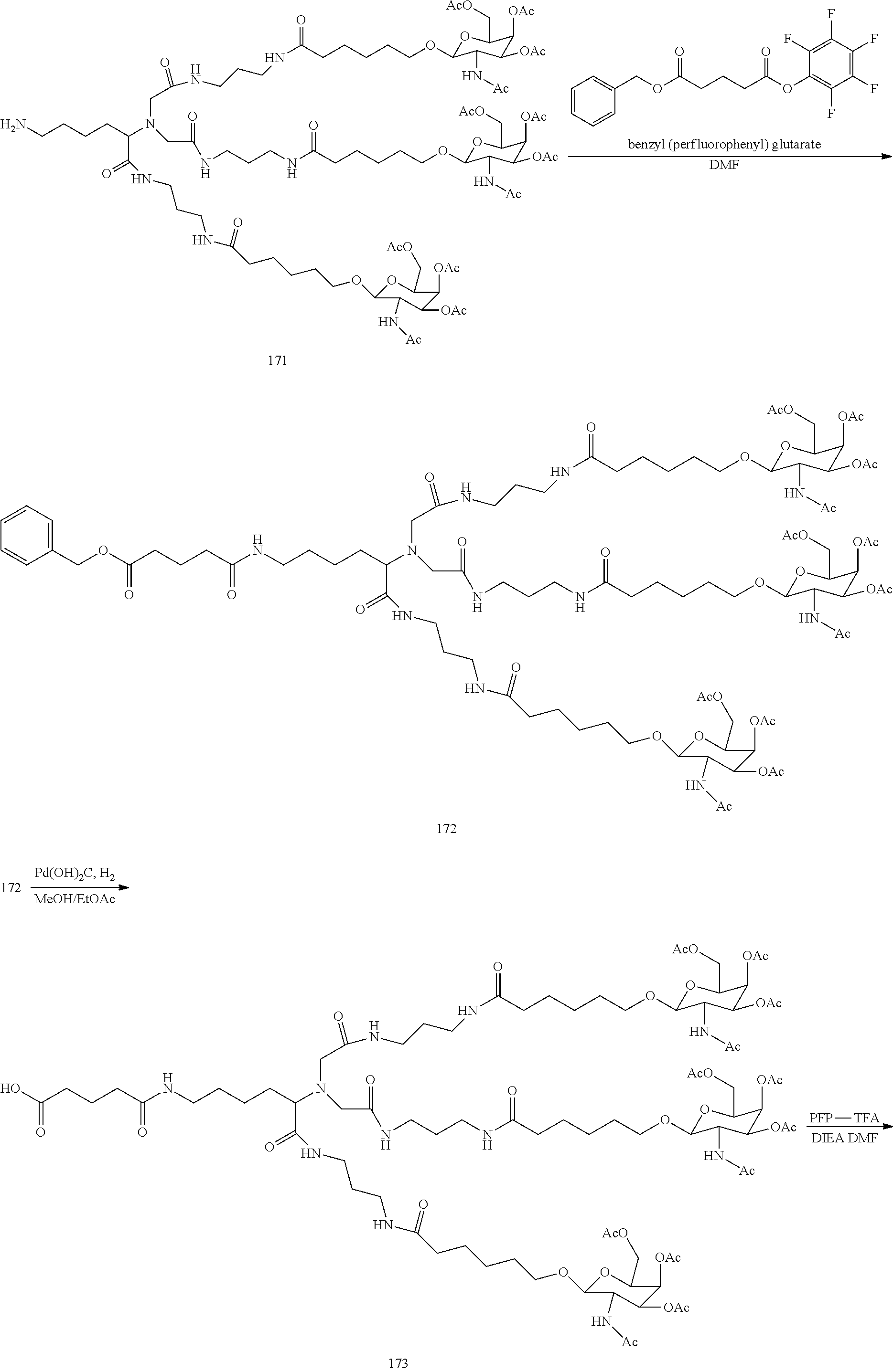

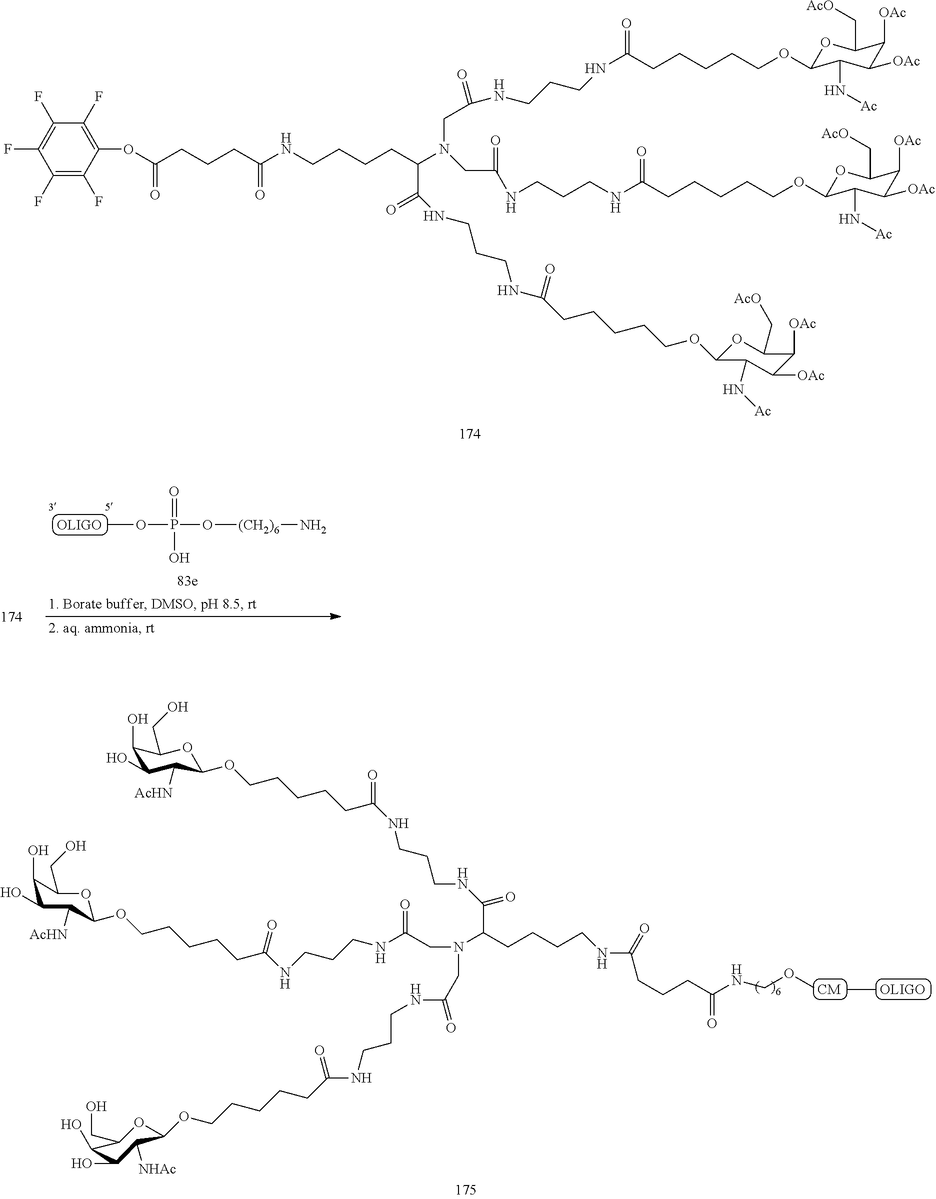

Compound 169 is commercially available. Compound 172 was prepared by addition of benzyl (perfluorophenyl) glutarate to compound 171. The benzyl (perfluorophenyl) glutarate was prepared by adding PFP-TFA and DIEA to 5-(benzyloxy)-5-oxopentanoic acid in DMF. Oligomeric compound 175, comprising a GalNAc₃-12 conjugate group, was prepared from compound 174 using the general procedures illustrated in Example 46. The GalNAc3 cluster portion of the conjugate group GalNAc₃-12 (GalNAc₃-12ₐ) can be combined with any cleavable moiety to provide a variety of conjugate groups. In a certain embodiments, the cleavable moiety is —P(═O)(OH)-A_{d}-P(═O)(OH)—. The structure of GalNAc₃-12 (GalNAc₃-12ₐ-CM-) is shown below:

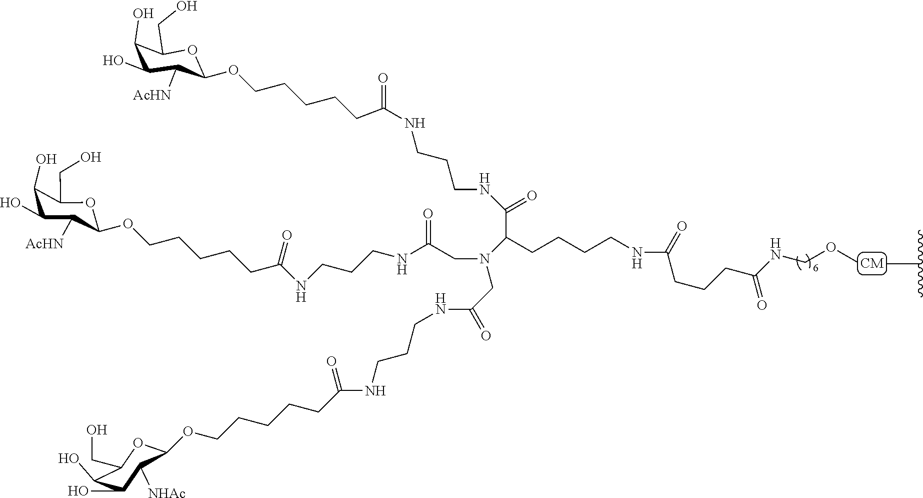

### Example 62: Preparation of Oligomeric Compound 180 Comprising GalNAc₃-13

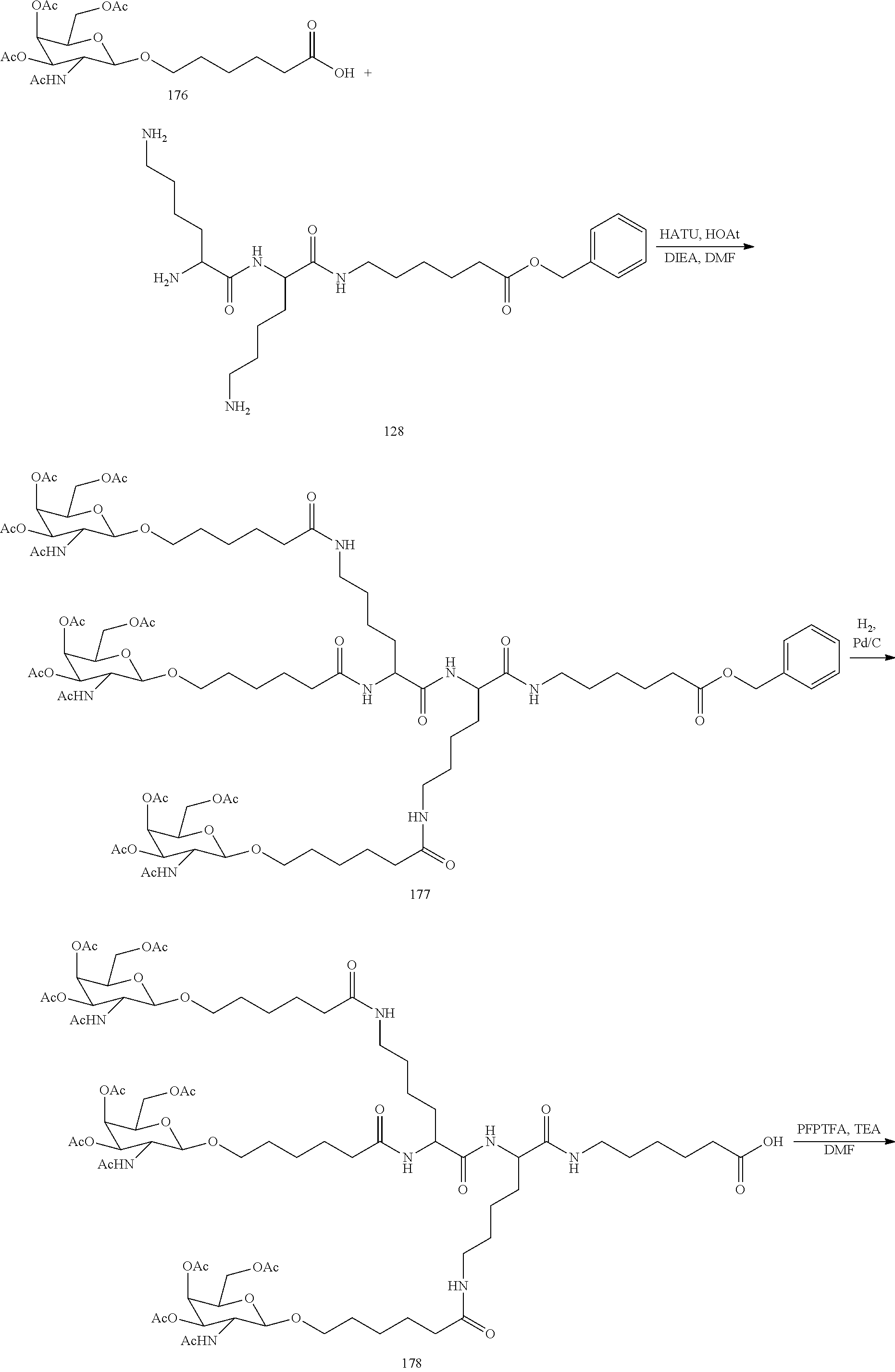

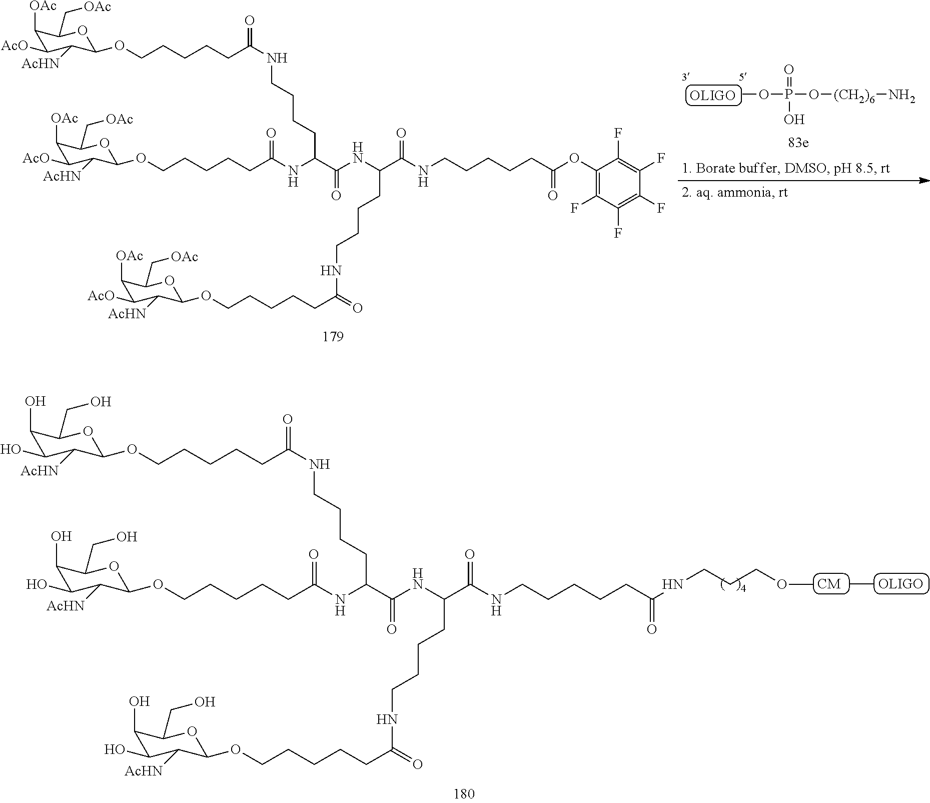

Compound 176 was prepared using the general procedure shown in Example 2. Oligomeric compound 180, comprising a GalNAc₃-13 conjugate group, was prepared from compound 177 using the general procedures illustrated in Example 49. The GalNAc3 cluster portion of the conjugate group GalNAc₃-13 (GalNAc₃-13ₐ) can be combined with any cleavable moiety to provide a variety of conjugate groups. In a certain embodiments, the cleavable moiety is —P(═O)(OH)-A_{d}-P(═O)(OH)—. The structure of GalNAc₃-13 (GalNAc₃-13ₐ-CM-) is shown below:

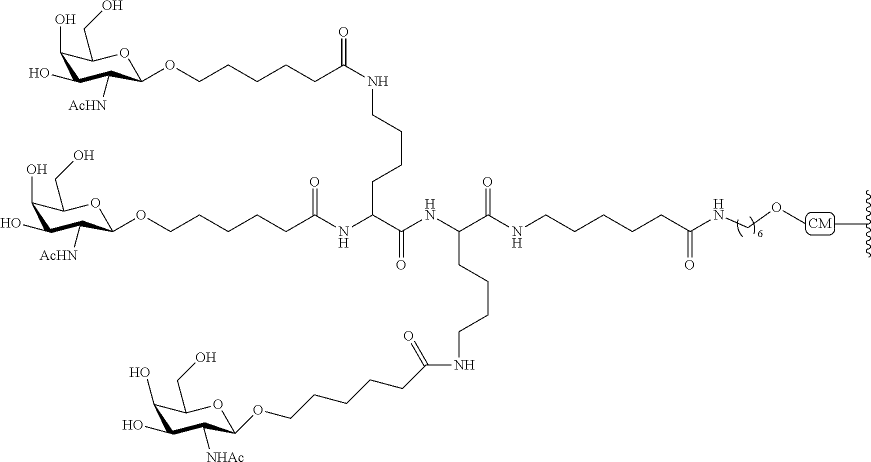

### Example 63: Preparation of Oligomeric Compound 188 Comprising GalNAc₃-14

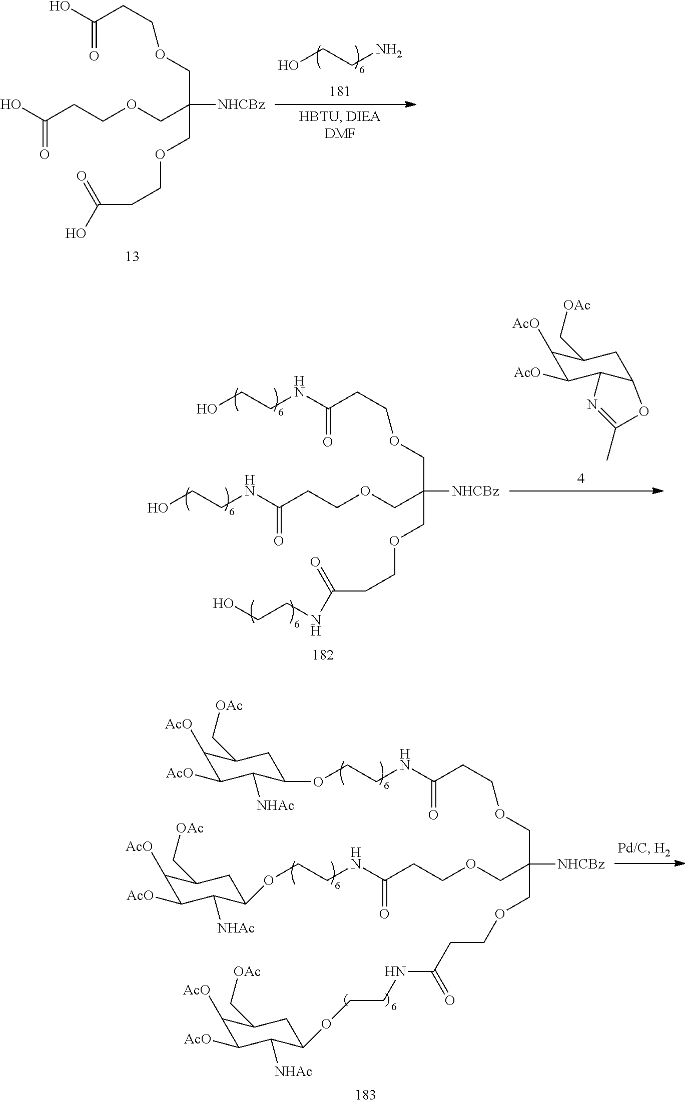

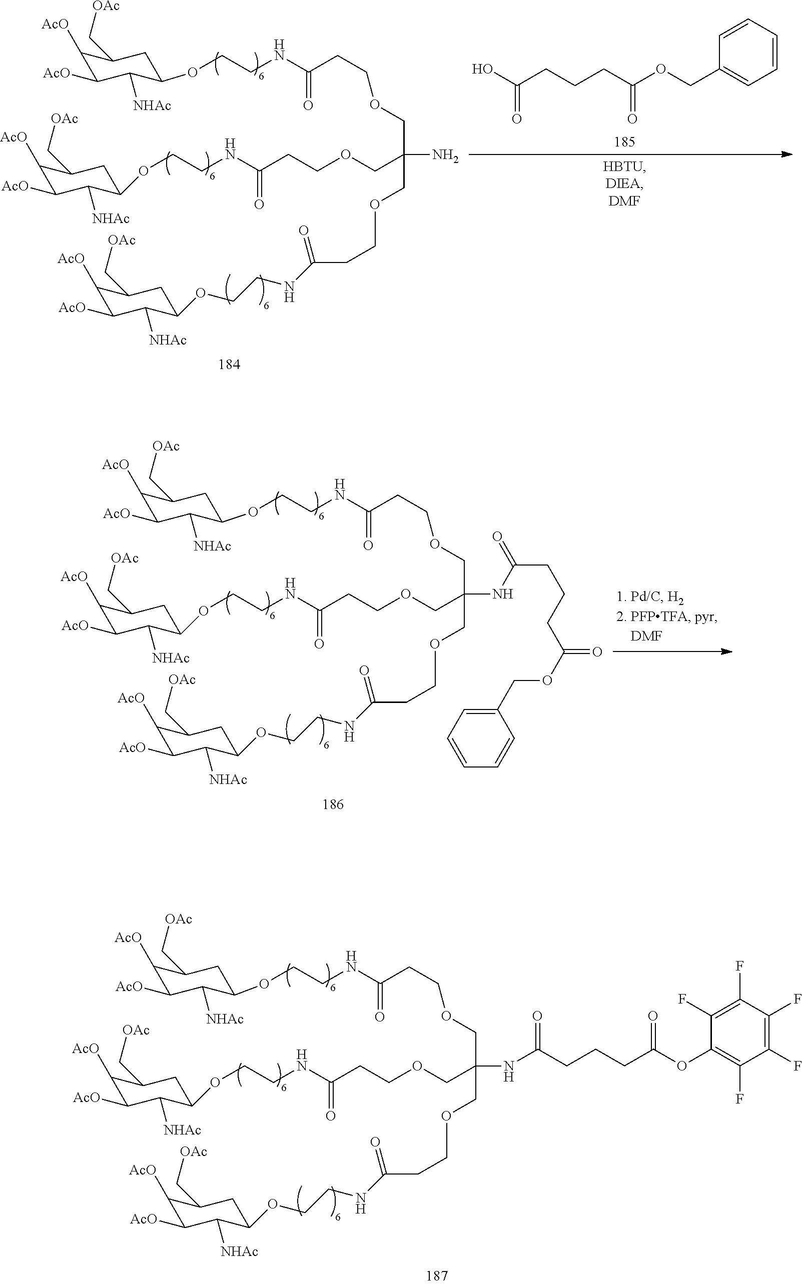

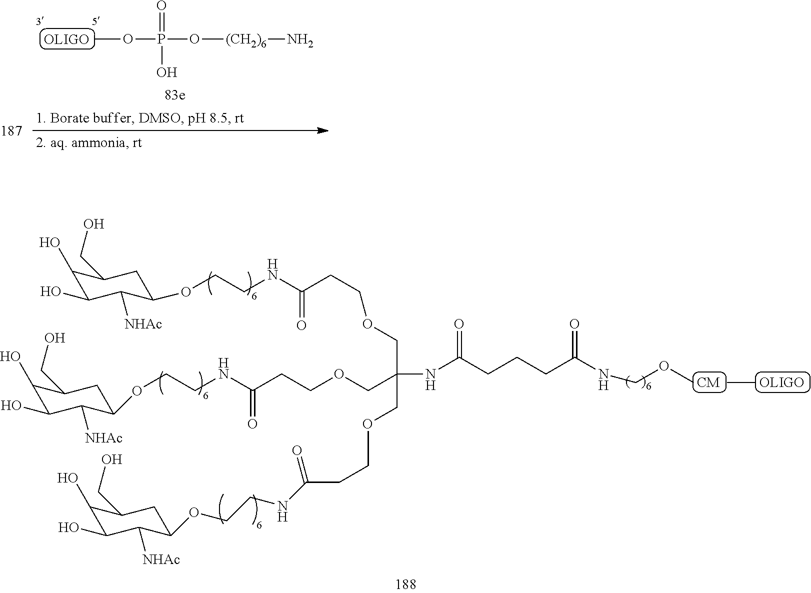

Compounds 181 and 185 are commercially available. Oligomeric compound 188, comprising a GalNAc₃-14 conjugate group, was prepared from compound 187 using the general procedures illustrated in Example 46. The GalNAc3 cluster portion of the conjugate group GalNAc₃-14 (GalNAc₃-14ₐ) can be combined with any cleavable moiety to provide a variety of conjugate groups. In certain embodiments, the cleavable moiety is —P(═O)(OH)-A_{d}-P(═O)(OH)—. The structure of GalNAc₃-14 (GalNAc₃-14ₐ-CM-) is shown below:

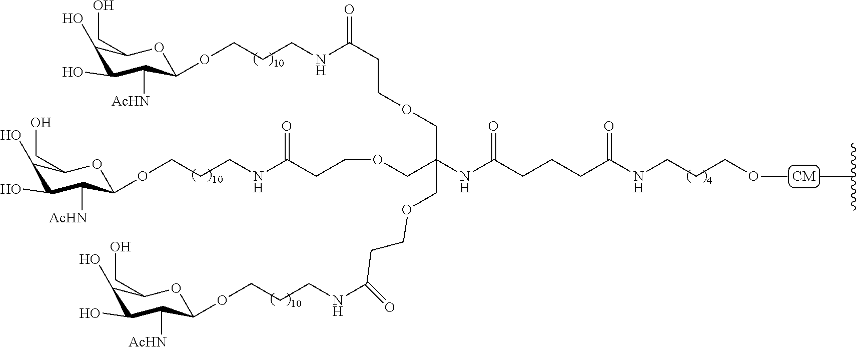

### Example 64: Preparation of Oligomeric Compound 197 Comprising GalNAc₃-15

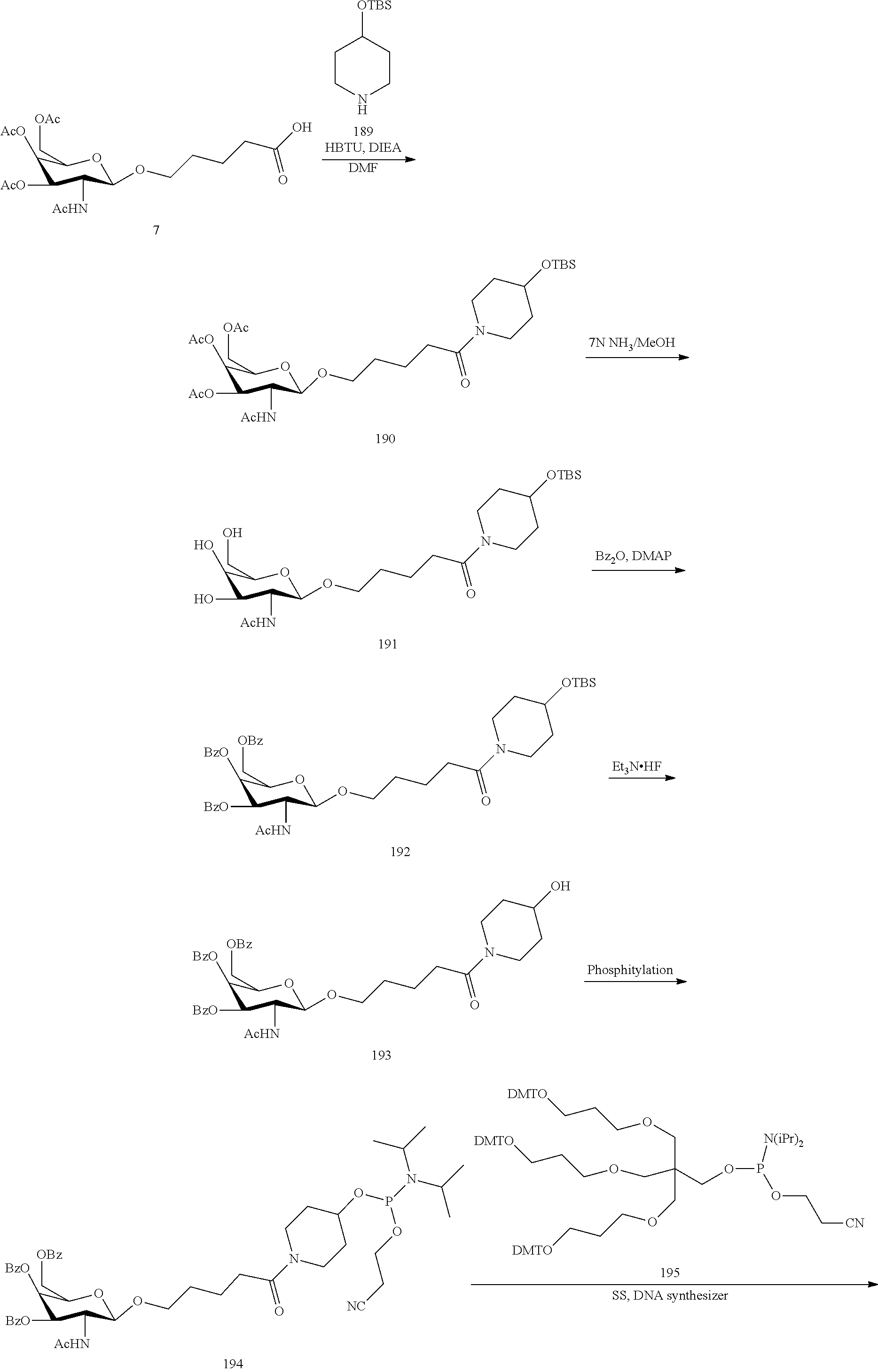

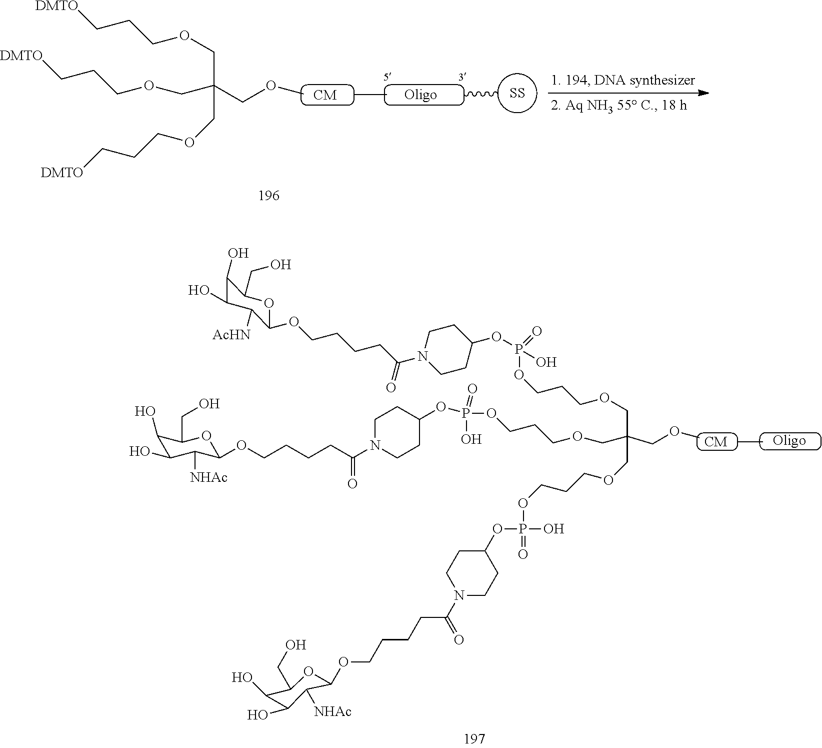

Compound 189 is commercially available. Compound 195 was prepared using the general procedure shown in Example 31. Oligomeric compound 197, comprising a GalNAc₃-15 conjugate group, was prepared from compounds 194 and 195 using standard oligonucleotide synthesis procedures. The GalNAc3 cluster portion of the conjugate group GalNAc₃-15 (GalNAc₃-15ₐ) can be combined with any cleavable moiety to provide a variety of conjugate groups. In certain embodiments, the cleavable moiety is —P(═O)(OH)-A_{d}-P(═O)(OH)—. The structure of GalNAc₃-15 (GalNAc₃-15ₐ-CM-) is shown below:

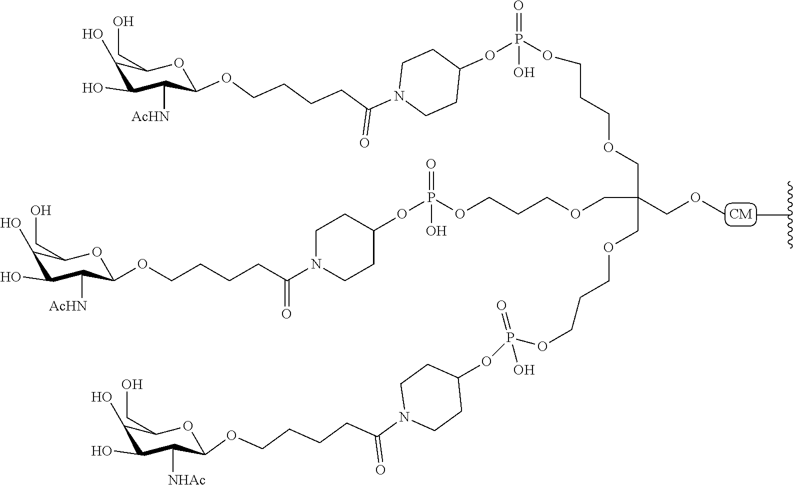

### Example 65: Dose-Dependent Study of Oligonucleotides Comprising a 5′-Conjugate Group (Comparison of GalNAc₃-3, 12, 13, 14, and 15) Targeting SRB-1 In Vivo

The oligonucleotides listed below were tested in a dose-dependent study for antisense inhibition of SRB-1 in mice. Unconjugated ISIS 353382 was included as a standard. Each of the GalNAc3 conjugate groups was attached at the 5′ terminus of the respective oligonucleotide by a phosphodiester linked 2-deoxyadenosine nucleoside (cleavable moiety).

TABLE 54
Modified ASOs targeting SRB-1

| | | | SEQ |
|---|---|---|---|
| ISIS No. | Sequences (5′ to 3′) | Conjugate | ID No. |
| 353382 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ^{m}Cₑₛ^{m}Cₑₛ | none | 829 |
| | TₑₛTₑ | | |
| 661161 | GalNAc₃-3ₐ-_{o′} | GalNAc₃-3 | 831 |
| | A_{do}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds} | | |
| | Tₑₛ^{m}Cₑₛ^{m}CₑₛTₑₛTₑ | | |
| 671144 | GalNAc₃-12ₐ-_{o′} | GalNAc₃-12 | 831 |
| | A_{do}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds} | | |
| | Tₑₛ^{m}Cₑₛ^{m}CₑₛTₑₛTₑ | | |
| 670061 | GalNAc₃-13ₐ-_{o′} | GalNAc₃-13 | 831 |
| | A_{do}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds} | | |
| | Tₑₛ^{m}Cₑₛ^{m}CₑₛTₑₛTₑ | | |
| 671261 | GalNAc₃-14ₐ-_{o′} | GalNAc₃-14 | 831 |
| | A_{do}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds} | | |
| | Tₑₛ^{m}Cₑₛ^{m}CₑₛTₑₛTₑ | | |
| 671262 | GalNAc₃-15ₐ-_{o′} | GalNAc₃-15 | 831 |
| | A_{do}Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds} | | |
| | Tₑₛ^{m}Cₑₛ^{m}CₑₛTₑₛTₑ | | |

Capital letters indicate the nucleobase for each nucleoside and ^{m}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-3ₐ was shown previously in Example 39. The structure of GalNAc3-12a was shown previously in Example 61. The structure of GalNAc₃-13ₐ was shown previously in Example 62. The structure of GalNAc₃-14ₐ was shown previously in Example 63. The structure of GalNAc₃-15ₐ was shown previously in Example 64.

### Treatment

Six to eight week old C57bl6 mice (Jackson Laboratory, Bar Harbor, ME) were injected subcutaneously once or twice at the dosage shown below with ISIS 353382, 661161, 671144, 670061, 671261, 671262, or with saline. Mice that were dosed twice received the second dose three days after the first dose. Each treatment group consisted of 4 animals. The mice were sacrificed 72 hours following the final administration to determine the liver SRB-1 mRNA levels using real-time PCR and RIBOGREEN® RNA quantification reagent (Molecular Probes, Inc. Eugene, OR) according to standard protocols. The results below are presented as the average percent of SRB-1 mRNA levels for each treatment group, normalized to the saline control.

As illustrated in Table 55, treatment with antisense oligonucleotides lowered SRB-1 mRNA levels in a dose-dependent manner. No significant differences in target knockdown were observed between animals that received a single dose and animals that received two doses (see ISIS 353382 dosages 30 and 2×15 mg/kg; and ISIS 661161 dosages 5 and 2×2.5 mg/kg). The antisense oligonucleotides comprising the phosphodiester linked GalNAc₃-3, 12, 13, 14, and 15 conjugates showed substantial improvement in potency compared to the unconjugated antisense oligonucleotide (ISIS 335382).

TABLE 55
SRB-1 mRNA (% Saline)

| ISIS | Dosage | SRB-1 mRNA | ED₅₀ | |
|---|---|---|---|---|
| No. | (mg/kg) | (% Saline) | (mg/kg) | Conjugate |
| Saline | n/a | 100.0 | n/a | n/a |
| 353382 | 3 | 85.0 | 22.4 | none |
| | 10 | 69.2 | | |
| | 30 | 34.2 | | |
| | 2 × 15 | 36.0 | | |
| 661161 | 0.5 | 87.4 | 2.2 | GalNAc₃-3 |
| | 1.5 | 59.0 | | |
| | 5 | 25.6 | | |
| | 2 × 2.5 | 27.5 | | |
| | 15 | 17.4 | | |
| 671144 | 0.5 | 101.2 | 3.4 | GalNAc₃-12 |
| | 1.5 | 76.1 | | |
| | 5 | 32.0 | | |
| | 15 | 17.6 | | |
| 670061 | 0.5 | 94.8 | 2.1 | GalNAc₃-13 |
| | 1.5 | 57.8 | | |
| | 5 | 20.7 | | |
| | 15 | 13.3 | | |
| 671261 | 0.5 | 110.7 | 4.1 | GalNAc₃-14 |
| | 1.5 | 81.9 | | |
| | 5 | 39.8 | | |
| | 15 | 14.1 | | |
| 671262 | 0.5 | 109.4 | 9.8 | GalNAc₃-15 |
| | 1.5 | 99.5 | | |
| | 5 | 69.2 | | |
| | 15 | 36.1 | | |

Liver transaminase levels, alanine aminotransferase (ALT) and aspartate aminotransferase (AST), in serum were measured relative to saline injected mice using standard protocols. Total bilirubin and BUN were also evaluated. The changes in body weights were evaluated with no significant differences from the saline group (data not shown). ALTs, ASTs, total bilirubin and BUN values are shown in Table 56 below.

TABLE 56

| | | | | Total | | |
|---|---|---|---|---|---|---|
| ISIS | Dosage | ALT | AST | Bilirubin | BUN | |
| No. | (mg/kg) | (U/L) | (U/L) | (mg/dL) | (mg/dL) | Conjugate |
| Saline | n/a | 28 | 60 | 0.1 | 39 | n/a |
| 353382 | 3 | 30 | 77 | 0.2 | 36 | none |
| | 10 | 25 | 78 | 0.2 | 36 | |
| | 30 | 28 | 62 | 0.2 | 35 | |
| | 2 × 15 | 22 | 59 | 0.2 | 33 | |
| 661161 | 0.5 | 39 | 72 | 0.2 | 34 | GalNAc₃-3 |
| | 1.5 | 26 | 50 | 0.2 | 33 | |
| | 5 | 41 | 80 | 0.2 | 32 | |
| | 2 × 2.5 | 24 | 72 | 0.2 | 28 | |
| | 15 | 32 | 69 | 0.2 | 36 | |
| 671144 | 0.5 | 25 | 39 | 0.2 | 34 | GalNAc₃-12 |
| | 1.5 | 26 | 55 | 0.2 | 28 | |
| | 5 | 48 | 82 | 0.2 | 34 | |
| | 15 | 23 | 46 | 0.2 | 32 | |
| 670061 | 0.5 | 27 | 53 | 0.2 | 33 | GalNAc₃-13 |
| | 1.5 | 24 | 45 | 0.2 | 35 | |
| | 5 | 23 | 58 | 0.1 | 34 | |
| | 15 | 24 | 72 | 0.1 | 31 | |
| 671261 | 0.5 | 69 | 99 | 0.1 | 33 | GalNAc₃-14 |
| | 1.5 | 34 | 62 | 0.1 | 33 | |
| | 5 | 43 | 73 | 0.1 | 32 | |
| | 15 | 32 | 53 | 0.2 | 30 | |
| 671262 | 0.5 | 24 | 51 | 0.2 | 29 | GalNAc₃-15 |
| | 1.5 | 32 | 62 | 0.1 | 31 | |
| | 5 | 30 | 76 | 0.2 | 32 | |
| | 15 | 31 | 64 | 0.1 | 32 | |

### Example 66: Effect of Various Cleavable Moieties on Antisense Inhibition In Vivo by Oligonucleotides Targeting SRB-1 Comprising a 5′-GalNAc₃ Cluster

The oligonucleotides listed below were tested in a dose-dependent study for antisense inhibition of SRB-1 in mice. Each of the GalNAc3 conjugate groups was attached at the 5′ terminus of the respective oligonucleotide by a phosphodiester linked nucleoside (cleavable moiety (CM)).

TABLE 57
Modified ASOs targeting SRB-1

| ISIS | | GalNAc₃ | | SEQ |
|---|---|---|---|---|
| No. | Sequences (5′ to 3′) | Cluster | CM | ID No. |
| 661161 | GalNAc₃-3ₐ-_{o′}A_{do}Gₑₛ^{m}CₑₛTₑₛTₑₛ^{m}CₑₛA_{ds}G_{ds}T_{ds}^{m}C_{ds}A_{ds}T_{ds} | GalNAc₃₋3a | A_{d} | 831 |
| | G_{ds}A_{ds}^{m}C_{ds}T_{ds}Tₑₛ^{m}Cₑₛ^{m}CₑₛTₑₛTₑ | | | |
| 670699 | GalNAc₃-3ₐ-_{o′}T_{do}Gₑₛ^{m}CₑₒTₑₒTₑₒ^{m}CₑₒA_{ds}G_{ds}T_{ds}^{m}C_{ds}A_{ds}T_{ds} | GalNAc₃-3a | T_{d} | 834 |
| | G_{ds}A_{ds}^{m}C_{ds}T_{ds}Tₑₒ^{m}Cₑₒ^{m}CₑₛTₑₛTₑ | | | |
| 670700 | GalNAc₃-3ₐ-_{o′}AₑₒGₑₛ^{m}CₑₒTₑₒTₑₒ^{m}CₑₒA_{ds}G_{ds}T_{ds}^{m}C_{ds}A_{ds}T_{ds} | GalNAc₃-3a | Aₑ | 831 |
| | G_{ds}A_{ds}^{m}C_{ds}T_{ds}Tₑₒ^{m}Cₑₒ^{m}CₑₛTₑₛTₑ | | | |
| 670701 | GalNAc₃-3ₐ-_{o′}TₑₒGₑₛ^{m}CₑₒTₑₒTₑₒ^{m}CₑₒA_{ds}G_{ds}T_{ds}^{m}C_{ds}A_{ds}T_{ds} | GalNAc₃-3a | Tₑ | 834 |
| | G_{ds}A_{ds}^{m}C_{ds}T_{ds}Tₑₒ^{m}Cₑₒ^{m}CₑₛTₑₛTₑ | | | |
| 671165 | GalNAc₃-13ₐ-_{o′}A_{do}Gₑₛ^{m}CₑₒTₑₒTₑₒ^{m}CₑₒA_{ds}G_{ds}T_{ds}^{m}C_{ds}A_{ds}T_{ds} | GalNAc₃-13a | A_{d} | 831 |
| | G_{ds}A_{ds}^{m}C_{ds}T_{ds}Tₑₒ^{m}Cₑₒ^{m}CₑₛTₑₛTₑ | | | |

Capital letters indicate the nucleobase for each nucleoside and ^{m}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-3ₐ was shown previously in Example 39. The structure of GalNAc₃-13ₐ was shown previously in Example 62.

### Treatment

Six to eight week old C57bl6 mice (Jackson Laboratory, Bar Harbor, MVE) were injected subcutaneously once at the dosage shown below with ISIS 661161, 670699, 670700, 670701, 671165, or with saline. Each treatment group consisted of 4 animals. The mice were sacrificed 72 hours following the final administration to determine the liver SRB—I mRNA levels using real-time PCR and RIBOGREEN® RNA quantification reagent (Molecular Probes, Inc. Eugene, OR) according to standard protocols. The results below are presented as the average percent of SRB—I mRNA levels for each treatment group, normalized to the saline control.

As illustrated in Table 58, treatment with antisense oligonucleotides lowered SRB—I mRNA levels in a dose-dependent manner. The antisense oligonucleotides comprising various cleavable moieties all showed similar potencies.

TABLE 58
SRB-1 mRNA (% Saline)

| ISIS | Dosage | SRB-1 mRNA | GalNAc₃ | |
|---|---|---|---|---|
| No. | (mg/kg) | (% Saline) | Cluster | CM |
| Saline | n/a | 100.0 | n/a | n/a |
| 661161 | 0.5 | 87.8 | GalNAc₃-3a | A_{d} |
| | 1.5 | 61.3 | | |
| | 5 | 33.8 | | |
| | 15 | 14.0 | | |
| 670699 | 0.5 | 89.4 | GalNAc₃-3a | T_{d} |
| | 1.5 | 59.4 | | |
| | 5 | 31.3 | | |
| | 15 | 17.1 | | |
| 670700 | 0.5 | 79.0 | GalNAc₃-3a | Aₑ |
| | 1.5 | 63.3 | | |
| | 5 | 32.8 | | |
| | 15 | 17.9 | | |
| 670701 | 0.5 | 79.1 | GalNAc₃-3a | Tₑ |
| | 1.5 | 59.2 | | |
| | 5 | 35.8 | | |
| | 15 | 17.7 | | |
| 671165 | 0.5 | 76.4 | GalNAc₃-13a | A_{d} |
| | 1.5 | 43.2 | | |
| | 5 | 22.6 | | |
| | 15 | 10.0 | | |

Liver transaminase levels, alanine aminotransferase (ALT) and aspartate aminotransferase (AST), in serum were measured relative to saline injected mice using standard protocols. Total bilirubin and BUN were also evaluated. The changes in body weights were evaluated with no significant differences from the saline group (data not shown). ALTs, ASTs, total bilirubin and BUN values are shown in Table 56 below.

TABLE 59

| | | | | Total | | | |
|---|---|---|---|---|---|---|---|
| ISIS | Dosage | ALT | AST | Bilirubin | BUN | GalNAc₃ | |
| No. | (mg/kg) | (U/L) | (U/L) | (mg/dL) | (mg/dL) | Cluster | CM |
| Saline | n/a | 24 | 64 | 0.2 | 31 | n/a | n/a |
| 661161 | 0.5 | 25 | 64 | 0.2 | 31 | GalNAc₃-3a | A_{d} |
| | 1.5 | 24 | 50 | 0.2 | 32 | | |
| | 5 | 26 | 55 | 0.2 | 28 | | |
| | 15 | 27 | 52 | 0.2 | 31 | | |
| 670699 | 0.5 | 42 | 83 | 0.2 | 31 | GalNAc₃-3a | T_{d} |
| | 1.5 | 33 | 58 | 0.2 | 32 | | |
| | 5 | 26 | 70 | 0.2 | 29 | | |
| | 15 | 25 | 67 | 0.2 | 29 | | |
| 670700 | 0.5 | 40 | 74 | 0.2 | 27 | GalNAc₃-3a | Aₑ |
| | 1.5 | 23 | 62 | 0.2 | 27 | | |
| | 5 | 24 | 49 | 0.2 | 29 | | |
| | 15 | 25 | 87 | 0.1 | 25 | | |
| 670701 | 0.5 | 30 | 77 | 0.2 | 27 | GalNAc₃-3a | Tₑ |
| | 1.5 | 22 | 55 | 0.2 | 30 | | |
| | 5 | 81 | 101 | 0.2 | 25 | | |
| | 15 | 31 | 82 | 0.2 | 24 | | |
| 671165 | 0.5 | 44 | 84 | 0.2 | 26 | GalNAc₃-13a | A_{d} |
| | 1.5 | 47 | 71 | 0.1 | 24 | | |
| | 5 | 33 | 91 | 0.2 | 26 | | |
| | 15 | 33 | 56 | 0.2 | 29 | | |

### Example 67: Preparation of Oligomeric Compound 199 Comprising GalNAc₃-16

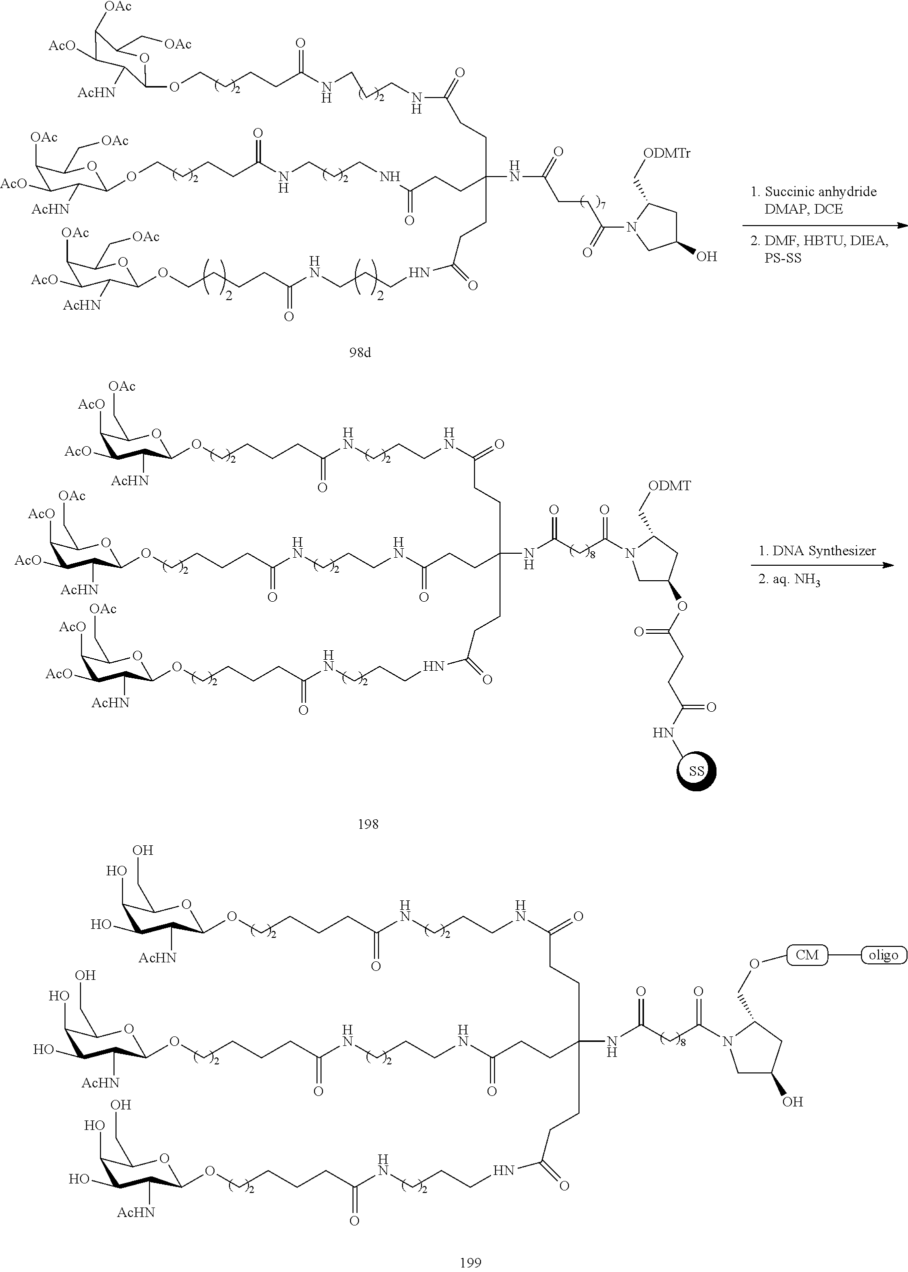

Oligomeric compound 199, comprising a GalNAc₃-16 conjugate group, is prepared using the general procedures illustrated in Examples 7 and 9. The GalNAc3 cluster portion of the conjugate group GalNAc₃-16 (GalNAc₃-16ₐ) can be combined with any cleavable moiety to provide a variety of conjugate groups. In certain embodiments, the cleavable moiety is —P(═O)(OH)-A_{d}-P(═O)(OH)—. The structure of GalNAc₃-16 (GalNAc₃-16ₐ-CM-) is shown below:

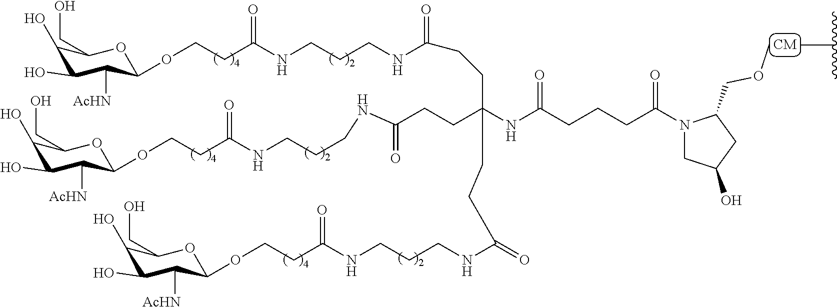

### Example 68: Preparation of Oligomeric Compound 200 Comprising GalNAc₃-17

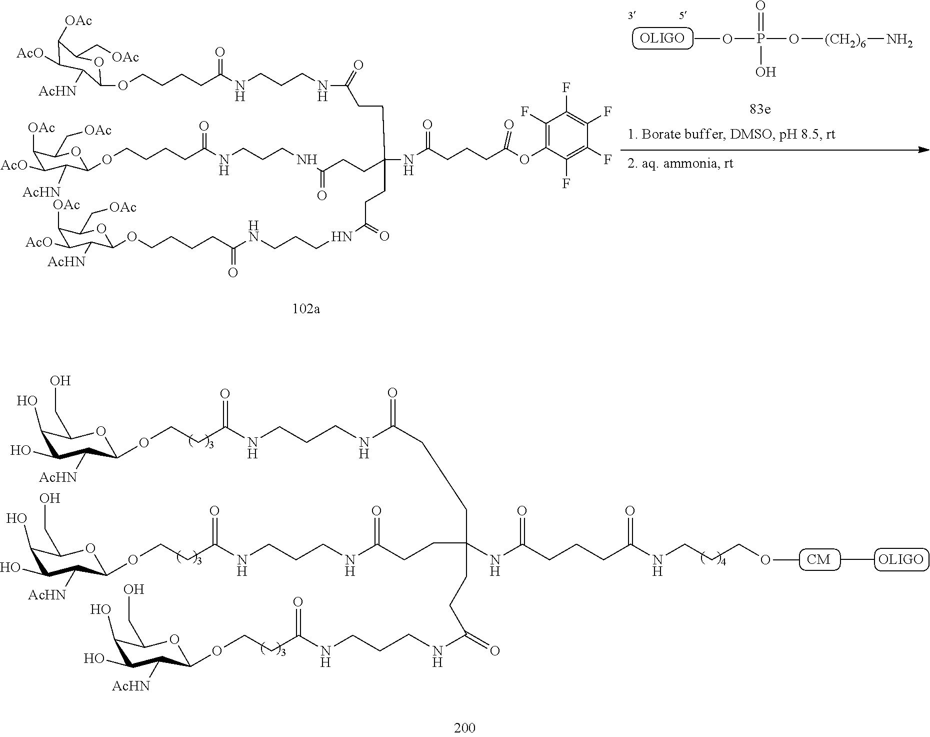

Oligomeric compound 200, comprising a GalNAc₃-17 conjugate group, was prepared using the general procedures illustrated in Example 46. The GalNAc3 cluster portion of the conjugate group GalNAc₃-17 (GalNAc₃-17ₐ) can be combined with any cleavable moiety to provide a variety of conjugate groups. In certain embodiments, the cleavable moiety is —P(═O)(OH)-A_{d}-P(═O)(OH)—. The structure of GalNAc₃-17 (GalNAc₃-17ₐ-CM-) is shown below:

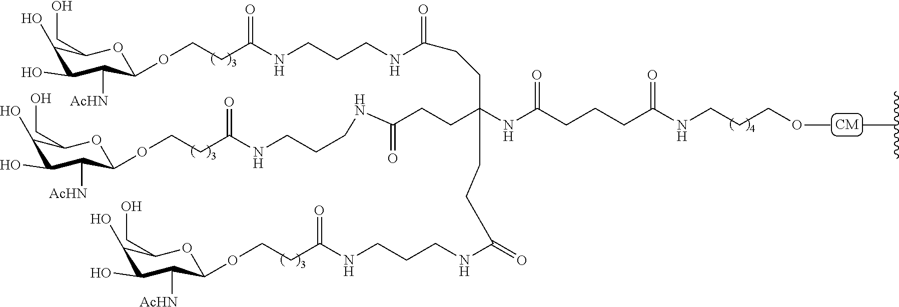

### Example 69: Preparation of Oligomeric Compound 201 Comprising GalNAc₃-18

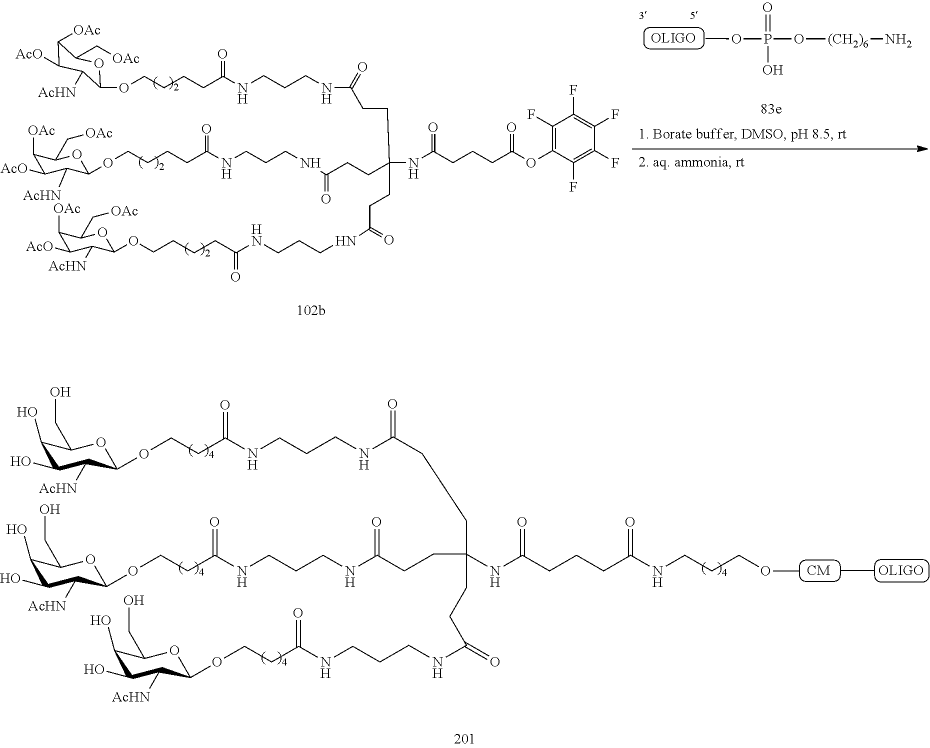

Oligomeric compound 201, comprising a GalNAc₃-18 conjugate group, was prepared using the general procedures illustrated in Example 46. The GalNAc3 cluster portion of the conjugate group GalNAc₃-18 (GalNAc₃-18ₐ) can be combined with any cleavable moiety to provide a variety of conjugate groups. In certain embodiments, the cleavable moiety is —P(═O)(OH)-A_{d}-P(═O)(OH)—. The structure of GalNAc₃-18 (GalNAc₃-18ₐ-CM-) is shown below:

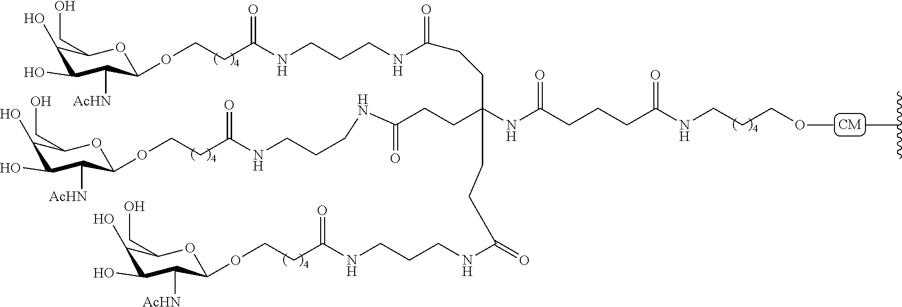

### Example 70: Preparation of Oligomeric Compound 204 Comprising GalNAc₃-19

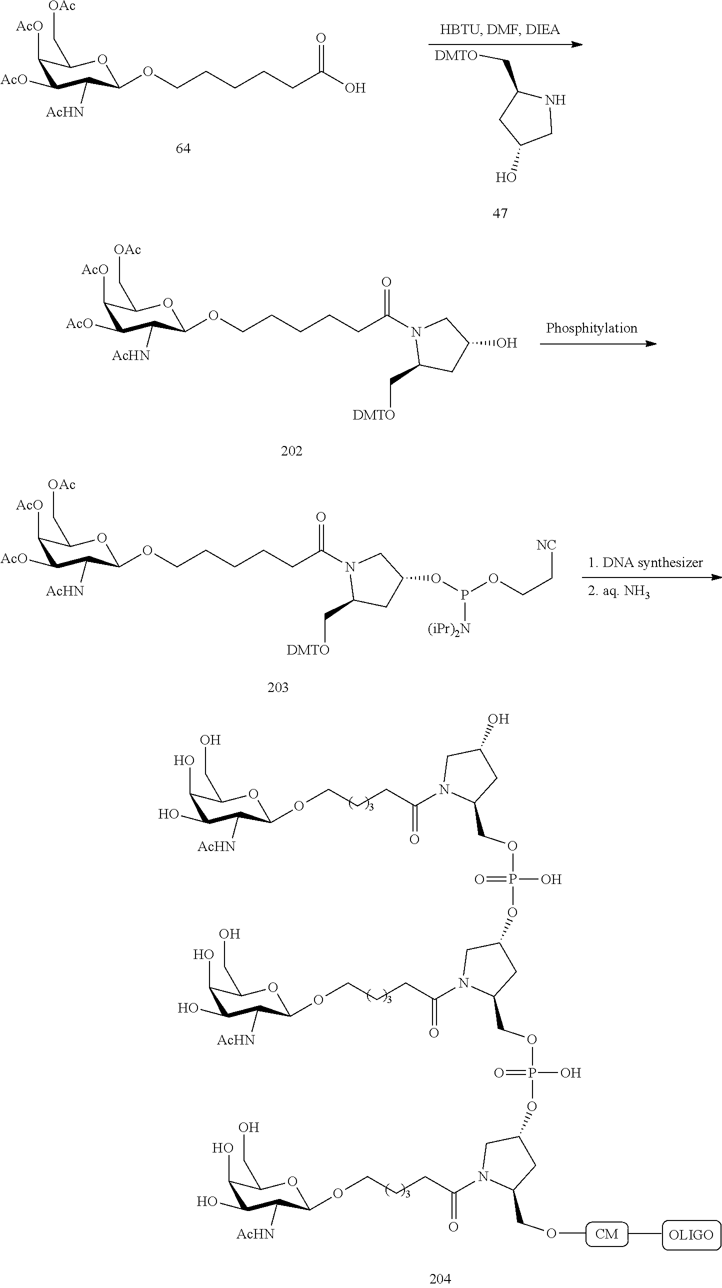

Oligomeric compound 204, comprising a GalNAc₃-19 conjugate group, was prepared from compound 64 using the general procedures illustrated in Example 52. The GalNAc3 cluster portion of the conjugate group GalNAc₃-19 (GalNAc₃-19ₐ) can be combined with any cleavable moiety to provide a variety of conjugate groups. In certain embodiments, the cleavable moiety is —P(═O)(OH)-A_{d}-P(═O)(OH)—. The structure of GalNAc₃-19 (GalNAc₃-19ₐ-CM-) is shown below:

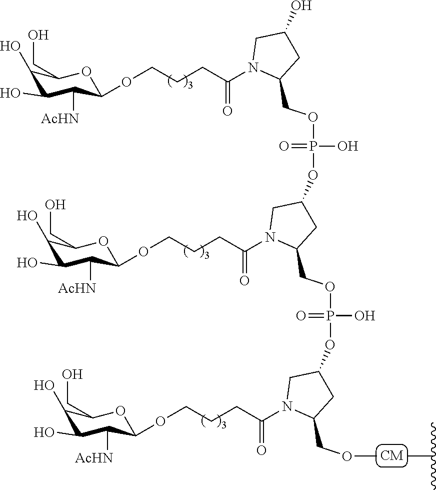

### Example 71: Preparation of Oligomeric Compound 210 Comprising GalNAc₃-20

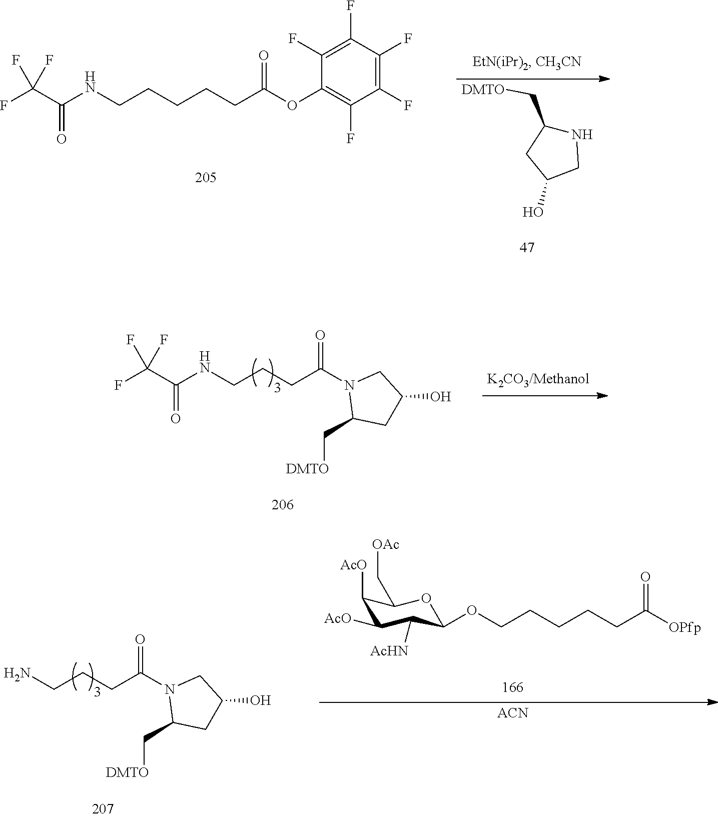

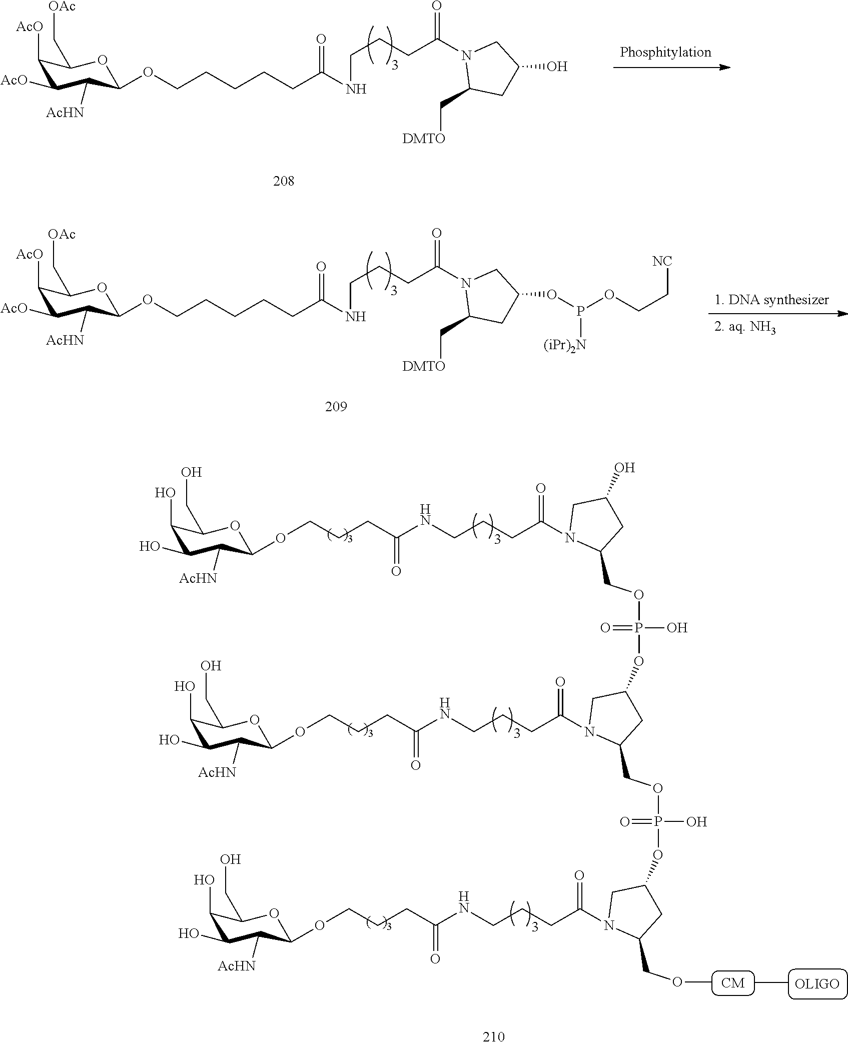

Compound 205 was prepared by adding PFP-TFA and DIEA to 6-(2,2,2-trifluoroacetamido)hexanoic acid in acetonitrile, which was prepared by adding triflic anhydride to 6-aminohexanoic acid. The reaction mixture was heated to 80° C., then lowered to rt. Oligomeric compound 210, comprising a GalNAc₃-20 conjugate group, was prepared from compound 208 using the general procedures illustrated in Example 52. The GalNAc3 cluster portion of the conjugate group GalNAc₃-20 (GalNAc₃-20ₐ) can be combined with any cleavable moiety to provide a variety of conjugate groups.

In certain embodiments, the cleavable moiety is —P(═O)(OH)-A_{d}-P(═O)(OH)—. The structure of GalNAc₃-20 (GalNAc₃-20ₐ-CM-) is shown below:

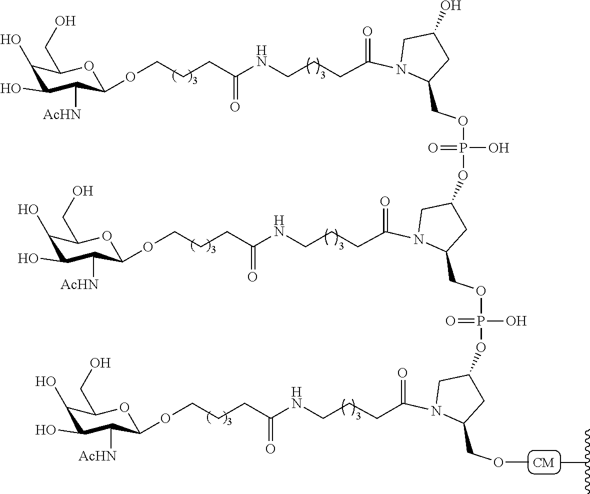

### Example 72: Preparation of Oligomeric Compound 215 Comprising GalNAc₃-21

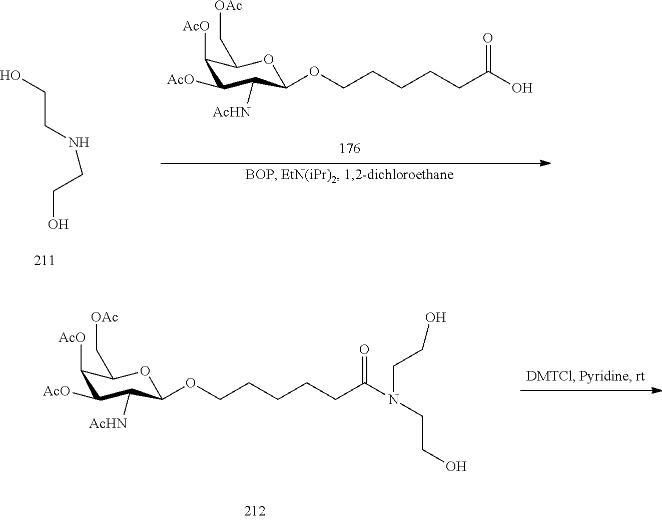

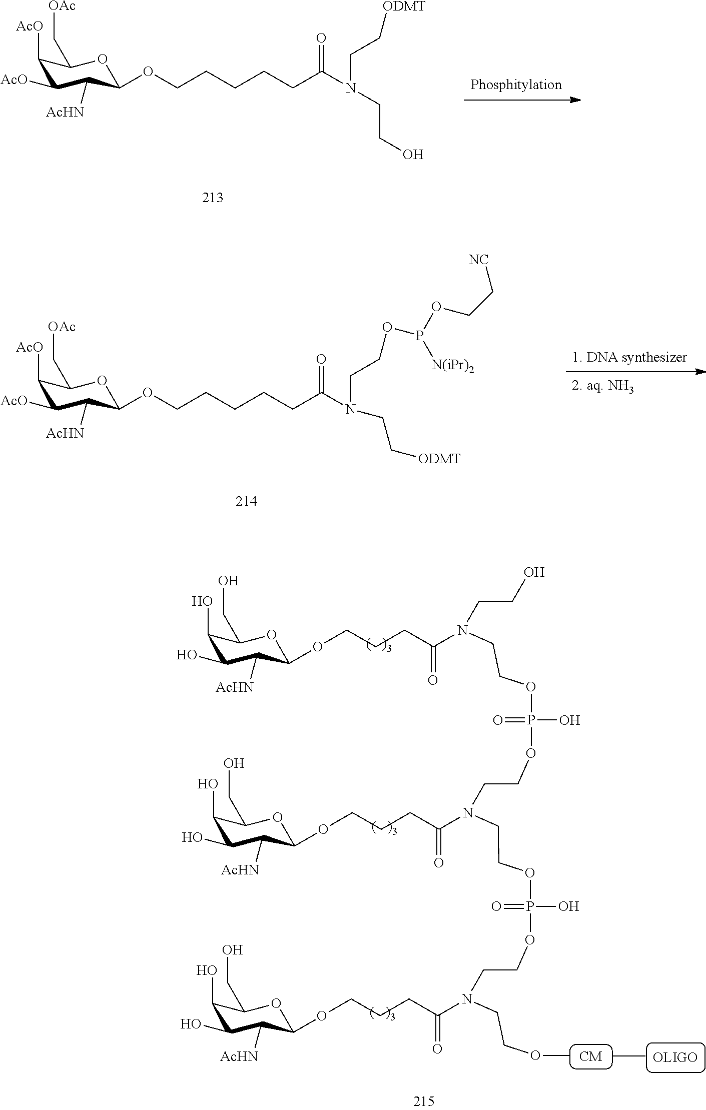

Compound 211 is commercially available. Oligomeric compound 215, comprising a GalNAc₃-21 conjugate group, was prepared from compound 213 using the general procedures illustrated in Example 52. The GalNAc3 cluster portion of the conjugate group GalNAc₃-21 (GalNAc₃-21ₐ) can be combined with any cleavable moiety to provide a variety of conjugate groups. In certain embodiments, the cleavable moiety is —P(═O)(OH)-A_{d}-P(═O)(OH)—. The structure of GalNAc₃-21 (GalNAc₃-21ₐ-CM-) is shown below:

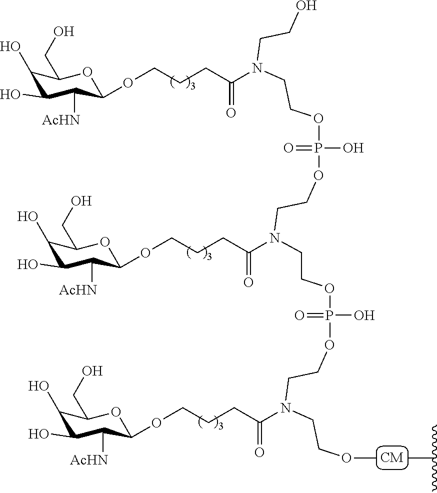

### Example 73: Preparation of Oligomeric Compound 221 Comprising GalNAc₃-22

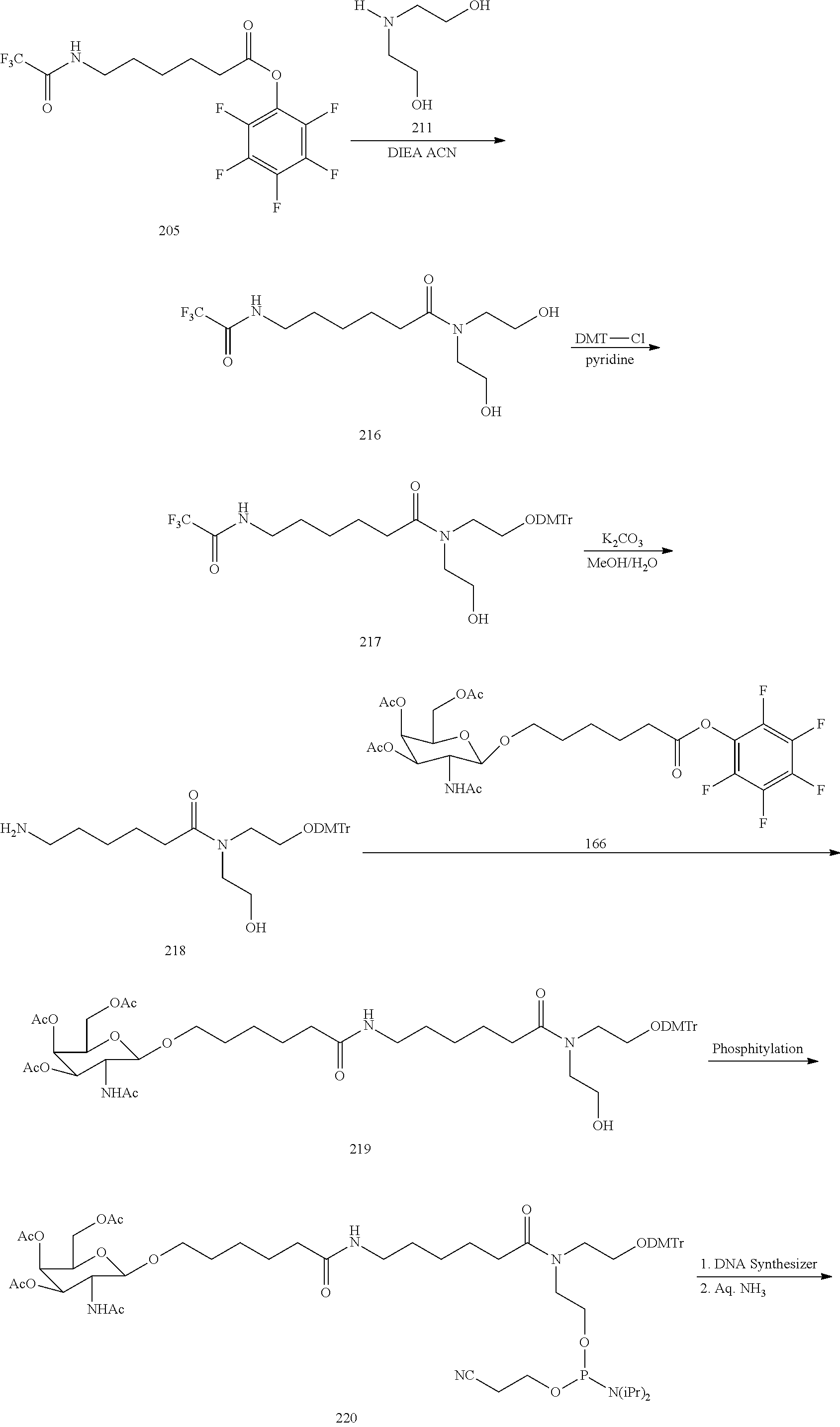

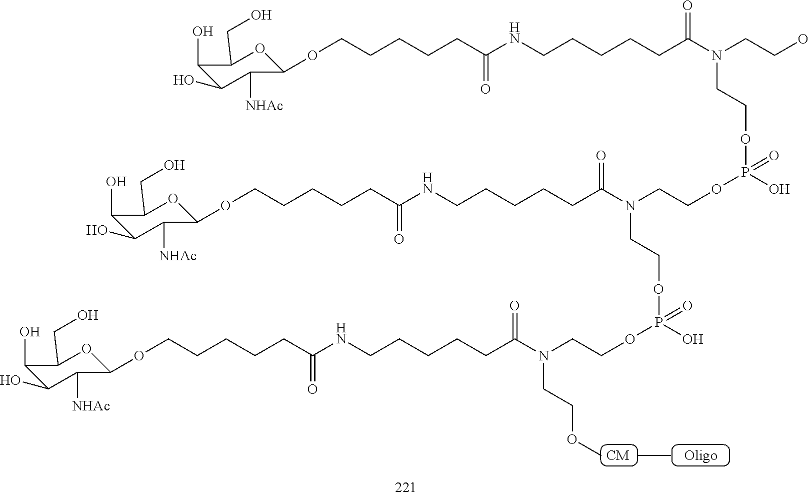

Compound was prepare from compound using diisopropylammonium tetrazolide. Oligomeric compound 221, comprising a GalNAc₃-21 conjugate group, is prepared from compound 220 using the general procedure illustrated in Example 52. The GalNAc3 cluster portion of the conjugate group GalNAc₃-22 (GalNAc₃-22ₐ) can be combined with any cleavable moiety to provide a variety of conjugate groups. In certain embodiments, the cleavable moiety is —P(═O)(OH)-A_{d}-P(═O)(OH)—. The structure of GalNAc₃-22 (GalNAc₃-22ₐ-CM-) is shown below:

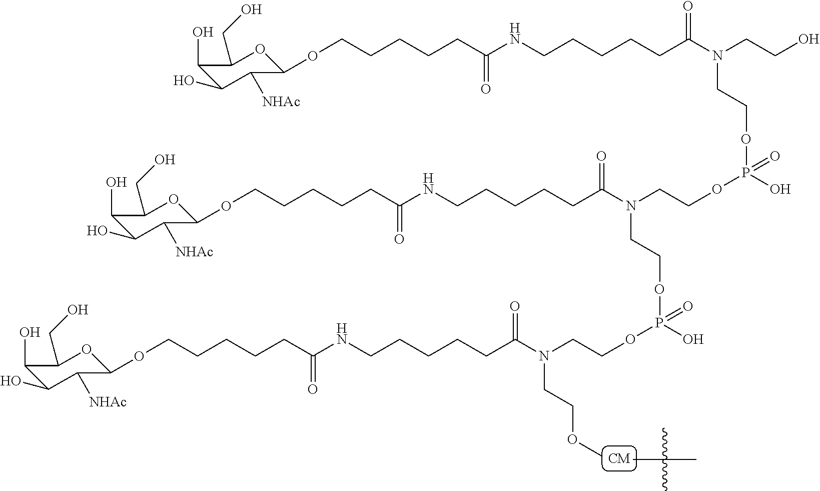

### Example 74: Effect of Various Cleavable Moieties on Antisense Inhibition In Vivo by Oligonucleotides Targeting SRB-1 Comprising a 5′-GalNAc₃ Conjugate

The oligonucleotides listed below were tested in a dose-dependent study for antisense inhibition of SRB-1 in mice. Each of the GalNAc3 conjugate groups was attached at the 5′ terminus of the respective oligonucleotide.

TABLE 60
Modified ASOs targeting SRB-1

| ISIS | | GalNAc₃ | | SEQ |
|---|---|---|---|---|
| No. | Sequences (5′ to 3′) | Cluster | CM | ID No. |
| 353382 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ | n/a | n/a | 829 |
| | ^{m}Cₑₛ^{m}CₑₛTₑₛTₑ | | | |
| 661161 | GalNAc₃-3ₐ-_{o′}A_{do}Gₑₛ^{m}CₑₛTₑₛTₑₛ^{m}CₑₛA_{ds}G_{ds}T_{ds}^{m}C_{ds}A_{ds}T_{ds} | GalNAc₃-3a | A_{d} | 831 |
| | G_{ds}A_{ds}^{m}C_{ds}T_{ds}Tₑₛ^{m}Cₑₛ^{m}CₑₛTₑₛTₑ | | | |
| 666904 | GalNAc₃-3ₐ-_{o′}Gₑₛ^{m}CₑₛTₑₛTₑₛ^{m}CₑₛA_{ds}G_{ds}T_{ds}^{m}C_{ds}A_{ds}T_{ds} | GalNAc₃-3a | PO | 829 |
| | G_{ds}A_{ds}^{m}C_{ds}T_{ds}Tₑₛ^{m}Cₑₛ^{m}CₑₛTₑₛTₑ | | | |
| 675441 | GalNAc₃-17ₐ-_{o′}A_{do}Gₑₛ^{m}CₑₛTₑₛTₑₛ^{m}CₑₛA_{ds}G_{ds}T_{ds}^{m}C_{ds}A_{ds}T_{ds} | GalNAc₃-17a | A_{d} | 831 |
| | G_{ds}A_{ds}^{m}C_{ds}T_{ds}Tₑₛ^{m}Cₑₛ^{m}CₑₛTₑₛTₑ | | | |
| 675442 | GalNAc₃-18ₐ-_{o′}A_{do}Gₑₛ^{m}CₑₛTₑₛTₑₛ^{m}CₑₛA_{ds}G_{ds}T_{ds}^{m}C_{ds}A_{ds}T_{ds} | GalNAc₃-18a | A_{d} | 831 |
| | G_{ds}A_{ds}^{m}C_{ds}T_{ds}Tₑₛ^{m}Cₑₛ^{m}CₑₛTₑₛTₑ | | | |

In all tables, capital letters indicate the nucleobase for each nucleoside and ^{m}C indicates a 5-methyl cytosine. Subscripts: “e” indicates a 2′-MOE modified nucleoside; “d” indicates a β-D-2′-deoxyribonucleoside; “s” indicates a phosphorothioate internucleoside linkage (PS); “o” indicates a phosphodiester internucleoside linkage (PO); and “o′” indicates —O—P(═O)(OH)—. Conjugate groups are in bold.

The structure of GalNAc₃-3ₐ was shown previously in Example 39. The structure of GalNAc3-17a was shown previously in Example 68, and the structure of GalNAc₃-18ₐ was shown in Example 69.

### Treatment

Six to eight week old C57BL/6 mice (Jackson Laboratory, Bar Harbor, ME) were injected subcutaneously once at the dosage shown below with an oligonucleotide listed in Table 60 or with saline. Each treatment group consisted of 4 animals. The mice were sacrificed 72 hours following the final administration to determine the SRB-1 mRNA levels using real-time PCR and RIBOGREEN® RNA quantification reagent (Molecular Probes, Inc. Eugene, OR) according to standard protocols. The results below are presented as the average percent of SRB-1 mRNA levels for each treatment group, normalized to the saline control.

As illustrated in Table 61, treatment with antisense oligonucleotides lowered SRB-1 mRNA levels in a dose-dependent manner. The antisense oligonucleotides comprising a GalNAc conjugate showed similar potencies and were significantly more potent than the parent oligonucleotide lacking a GalNAc conjugate.

TABLE 61
SRB-1 mRNA (% Saline)

| ISIS | Dosage | SRB-1 mRNA | GalNAc₃ | |
|---|---|---|---|---|
| No. | (mg/kg) | (% Saline) | Cluster | CM |
| Saline | n/a | 100.0 | n/a | n/a |
| 353382 | 3 | 79.38 | n/a | n/a |
| | 10 | 68.67 | | |
| | 30 | 40.70 | | |
| 661161 | 0.5 | 79.18 | GalNAc₃-3a | A_{d} |
| | 1.5 | 75.96 | | |
| | 5 | 30.53 | | |
| | 15 | 12.52 | | |
| 666904 | 0.5 | 91.30 | GalNAc₃-3a | PO |
| | 1.5 | 57.88 | | |
| | 5 | 21.22 | | |
| | 15 | 16.49 | | |
| 675441 | 0.5 | 76.71 | GalNAc₃-17a | A_{d} |
| | 1.5 | 63.63 | | |
| | 5 | 29.57 | | |
| | 15 | 13.49 | | |
| 675442 | 0.5 | 95.03 | GalNAc₃-18a | A_{d} |
| | 1.5 | 60.06 | | |
| | 5 | 31.04 | | |
| | 15 | 19.40 | | |

Liver transaminase levels, alanine aminotransferase (ALT) and aspartate aminotransferase (AST), in serum were measured relative to saline injected mice using standard protocols. Total bilirubin and BUN were also evaluated. The change in body weights was evaluated with no significant change from the saline group (data not shown). ALTs, ASTs, total bilirubin and BUN values are shown in Table 62 below.

TABLE 62

| | | | | Total | | | |
|---|---|---|---|---|---|---|---|
| ISIS | Dosage | ALT | AST | Bilirubin | BUN | GalNAc₃ | |
| No. | (mg/kg) | (U/L) | (U/L) | (mg/dL) | (mg/dL) | Cluster | CM |
| Saline | n/a | 26 | 59 | 0.16 | 42 | n/a | n/a |
| 353382 | 3 | 23 | 58 | 0.18 | 39 | n/a | n/a |
| | 10 | 28 | 58 | 0.16 | 43 | | |
| | 30 | 20 | 48 | 0.12 | 34 | | |
| 661161 | 0.5 | 30 | 47 | 0.13 | 35 | GalNAc₃-3a | A_{d} |
| | 1.5 | 23 | 53 | 0.14 | 37 | | |
| | 5 | 26 | 48 | 0.15 | 39 | | |
| | 15 | 32 | 57 | 0.15 | 42 | | |
| 666904 | 0.5 | 24 | 73 | 0.13 | 36 | GalNAc₃-3a | PO |
| | 1.5 | 21 | 48 | 0.12 | 32 | | |
| | 5 | 19 | 49 | 0.14 | 33 | | |
| | 15 | 20 | 52 | 0.15 | 26 | | |
| 675441 | 0.5 | 42 | 148 | 0.21 | 36 | GalNAc₃-17a | A_{d} |
| | 1.5 | 60 | 95 | 0.16 | 34 | | |
| | 5 | 27 | 75 | 0.14 | 37 | | |
| | 15 | 24 | 61 | 0.14 | 36 | | |
| 675442 | 0.5 | 26 | 65 | 0.15 | 37 | GalNAc₃-18a | A_{d} |
| | 1.5 | 25 | 64 | 0.15 | 43 | | |
| | 5 | 27 | 69 | 0.15 | 37 | | |
| | 15 | 30 | 84 | 0.14 | 37 | | |

### Example 75: Pharmacokinetic Analysis of Oligonucleotides Comprising a M-Conjugate Group

The PK of the ASOs in Tables 54, 57 and 60 above was evaluated using liver samples that were obtained following the treatment procedures described in Examples 65, 66, and 74. The liver samples were minced and extracted using standard protocols and analyzed by IP-HPLC-MS alongside an internal standard. The combined tissue level (μg/g) of all metabolites was measured by integrating the appropriate UV peaks, and the tissue level of the full-length ASO missing the conjugate (“parent,” which is Isis No. 353382 in this case) was measured using the appropriate extracted ion chromatograms (EIC).

TABLE 63
PK Analysis in Liver

| | | | Parent | | |
|---|---|---|---|---|---|
| | | Total | ASO | | |
| | | Tissue | Tissue | | |
| | | Level | Level | | |
| ISIS | Dosage | by UV | by EIC | GalNAc₃ | |
| No. | (mg/kg) | (μg/g) | (μg/g) | Cluster | CM |
| 353382 | 3 | 8.9 | 8.6 | n/a | n/a |
| | 10 | 22.4 | 21.0 | | |
| | 30 | 54.2 | 44.2 | | |
| 661161 | 5 | 32.4 | 20.7 | GalNAc₃-3a | A_{d} |
| | 15 | 63.2 | 44.1 | | |
| 671144 | 5 | 20.5 | 19.2 | GalNAc₃-12a | A_{d} |
| | 15 | 48.6 | 41.5 | | |
| 670061 | 5 | 31.6 | 28.0 | GalNAc₃-13a | A_{d} |
| | 15 | 67.6 | 55.5 | | |
| 671261 | 5 | 19.8 | 16.8 | GalNAc₃-14a | A_{d} |
| | 15 | 64.7 | 49.1 | | |
| 671262 | 5 | 18.5 | 7.4 | GalNAc₃-15a | A_{d} |
| | 15 | 52.3 | 24.2 | | |
| 670699 | 5 | 16.4 | 10.4 | GalNAc₃-3a | T_{d} |
| | 15 | 31.5 | 22.5 | | |
| 670700 | 5 | 19.3 | 10.9 | GalNAc₃-3a | Aₑ |
| | 15 | 38.1 | 20.0 | | |
| 670701 | 5 | 21.8 | 8.8 | GalNAc₃-3a | Tₑ |
| | 15 | 35.2 | 16.1 | | |
| 671165 | 5 | 27.1 | 26.5 | GalNAc₃-13a | A_{d} |
| | 15 | 48.3 | 44.3 | | |
| 666904 | 5 | 30.8 | 24.0 | GalNAc₃-3a | PO |
| | 15 | 52.6 | 37.6 | | |
| 675441 | 5 | 25.4 | 19.0 | GalNAc₃-17a | A_{d} |
| | 15 | 54.2 | 42.1 | | |
| 675442 | 5 | 22.2 | 20.7 | GalNAc₃-18a | A_{d} |
| | 15 | 39.6 | 29.0 | | |

The results in Table 63 above show that there were greater liver tissue levels of the oligonucleotides comprising a GalNAc3 conjugate group than of the parent oligonucleotide that does not comprise a GalNAc3 conjugate group (ISIS 353382) 72 hours following oligonucleotide administration, particularly when taking into consideration the differences in dosing between the oligonucleotides with and without a GalNAc3 conjugate group. Furthermore, by 72 hours, 40-98% of each oligonucleotide comprising a GalNAc3 conjugate group was metabolized to the parent compound, indicating that the GalNAc3 conjugate groups were cleaved from the oligonucleotides.

### Example 76: Preparation of Oligomeric Compound 230 Comprising GalNAc₃-23

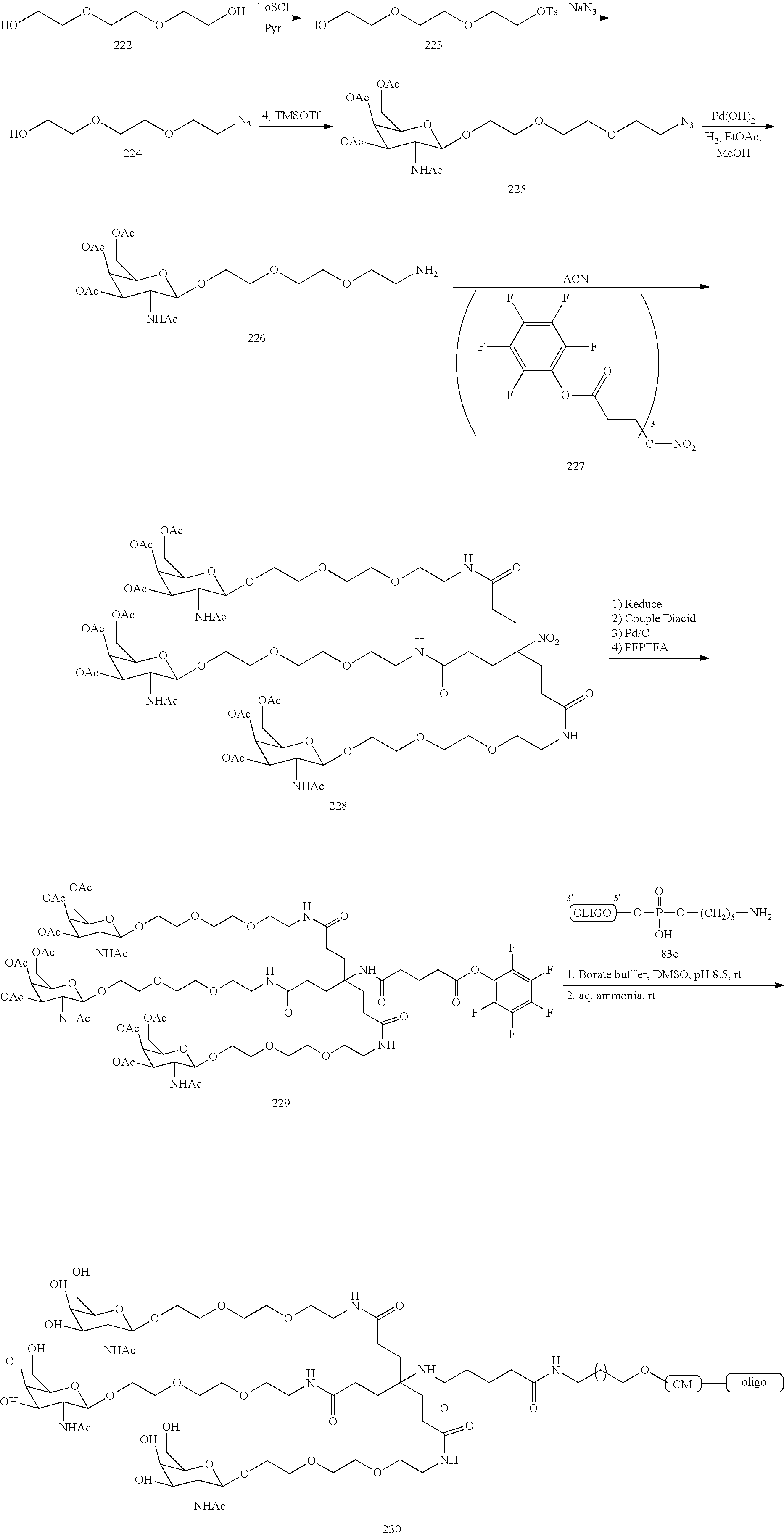

Compound 222 is commercially available. 44.48 ml (0.33 mol) of compound 222 was treated with tosyl chloride (25.39 g, 0.13 mol) in pyridine (500 mL) for 16 hours. The reaction was then evaporated to an oil, dissolved in EtOAc and washed with water, sat. NaHCO₃, brine, and dried over Na₂SO₄. The ethyl acetate was concentrated to dryness and purified by column chromatography, eluted with EtOAc/hexanes (1:1) followed by 10% methanol in CH₂C12 to give compound 223 as a colorless oil. LCMS and NMR were consistent with the structure. 10 g (32.86 mmol) of 1-Tosyltriethylene glycol (compound 223) was treated with sodium azide (10.68 g, 164.28 mmol) in DMSO (100 mL) at room temperature for 17 hours. The reaction mixture was then poured onto water, and extracted with EtOAc. The organic layer was washed with water three times and dried over Na₂SO₄. The organic layer was concentrated to dryness to give 5.3 g of compound 224 (92%). LCMS and NMR were consistent with the structure. 1-Azidotriethylene glycol (compound 224, 5.53 g, 23.69 mmol) and compound 4 (6 g, 18.22 mmol) were treated with 4A molecular sieves (5 g), and TMSOTf (1.65 ml, 9.11 mmol) in dichloromethane (100 mL) under an inert atmosphere. After 14 hours, the reaction was filtered to remove the sieves, and the organic layer was washed with sat. NaHCO₃, water, brine, and dried over Na₂SO₄. The organic layer was concentrated to dryness and purified by column chromatography, eluted with a gradient of 2 to 4% methanol in dichloromethane to give compound 225. LCMS and NMR were consistent with the structure. Compound 225 (11.9 g, 23.59 mmol) was hydrogenated in EtOAc/Methanol (4:1, 250 mL) over Pearlman's catalyst. After 8 hours, the catalyst was removed by filtration and the solvents removed to dryness to give compound 226. LCMS and NMR were consistent with the structure.

In order to generate compound 227, a solution of nitromethanetrispropionic acid (4.17 g, 15.04 mmol) and Hunig's base (10.3 ml, 60.17 mmol) in DMF (100 mL) were treated dropwise with pentaflourotrifluoro acetate (9.05 ml, 52.65 mmol). After 30 minutes, the reaction was poured onto ice water and extracted with EtOAc. The organic layer was washed with water, brine, and dried over Na₂SO₄. The organic layer was concentrated to dryness and then recrystallized from heptane to give compound 227 as a white solid. LCMS and NMR were consistent with the structure. Compound 227 (1.5 g, 1.93 mmol) and compound 226 (3.7 g, 7.74 mmol) were stirred at room temperature in acetonitrile (15 mL) for 2 hours. The reaction was then evaporated to dryness and purified by column chromatography, eluting with a gradient of 2 to 10% methanol in dichloromethane to give compound 228. LCMS and NMR were consistent with the structure. Compound 228 (1.7 g, 1.02 mmol) was treated with Raney Nickel (about 2 g wet) in ethanol (100 mL) in an atmosphere of hydrogen. After 12 hours, the catalyst was removed by filtration and the organic layer was evaporated to a solid that was used directly in the next step. LCMS and NMR were consistent with the structure. This solid (0.87 g, 0.53 mmol) was treated with benzylglutaric acid (0.18 g, 0.8 mmol), HBTU (0.3 g, 0.8 mmol) and DIEA (273.7 μl, 1.6 mmol) in DMF (5 mL). After 16 hours, the DMF was removed under reduced pressure at 65° C. to an oil, and the oil was dissolved in dichloromethane. The organic layer was washed with sat. NaHCO₃, brine, and dried over Na₂SO₄. After evaporation of the organic layer, the compound was purified by column chromatography and eluted with a gradient of 2 to 20% methanol in dichloromethane to give the coupled product. LCMS and NMR were consistent with the structure. The benzyl ester was deprotected with Pearlman's catalyst under a hydrogen atmosphere for 1 hour. The catalyst was them removed by filtration and the solvents removed to dryness to give the acid. LCMS and NMR were consistent with the structure. The acid (486 mg, 0.27 mmol) was dissolved in dry DMF (3 mL). Pyridine (53.61 μl, 0.66 mmol) was added and the reaction was purged with argon. Pentaflourotriflouro acetate (46.39 μl, 0.4 mmol) was slowly added to the reaction mixture. The color of the reaction changed from pale yellow to burgundy, and gave off a light smoke which was blown away with a stream of argon. The reaction was allowed to stir at room temperature for one hour (completion of reaction was confirmed by LCMS). The solvent was removed under reduced pressure (rotovap) at 70° C. The residue was diluted with DCM and washed with 1N NaHSO₄, brine, saturated sodium bicarbonate and brine again. The organics were dried over Na₂SO₄, filtered, and were concentrated to dryness to give 225 mg of compound 229 as a brittle yellow foam. LCMS and NMR were consistent with the structure.

Oligomeric compound 230, comprising a GalNAc₃-23 conjugate group, was prepared from compound 229 using the general procedure illustrated in Example 46. The GalNAc3 cluster portion of the GalNAc₃-23 conjugate group (GalNAc₃-23ₐ) can be combined with any cleavable moiety to provide a variety of conjugate groups. The structure of GalNAc₃-23 (GalNAc₃-23ₐ-CM) is shown below:

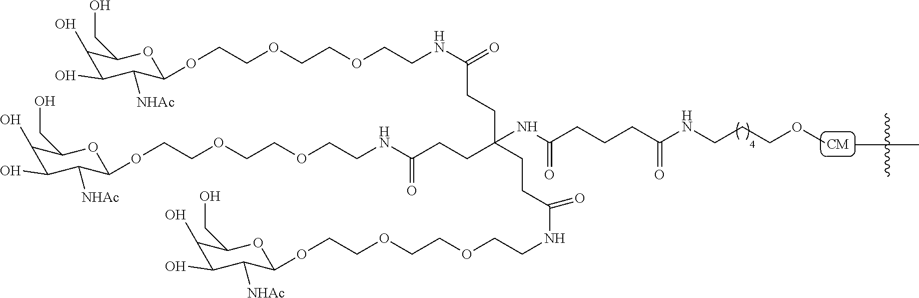

Example 77: Antisense Inhibition In Vivo by Oligonucleotides Targeting SRB-1 Comprising a GalNAc₃ Conjugate

The oligonucleotides listed below were tested in a dose-dependent study for antisense inhibition of SRB-1 in mice.

TABLE 64
Modified ASOs targeting SRB-1

| ISIS | | GalNAc₃ | | SEQ |
|---|---|---|---|---|
| No. | Sequences (5′ to 3′) | Cluster | CM | ID No. |
| 661161 | GalNAc₃-3ₐ-_{o′}A_{do}Gₑₛ^{m}CₑₛTₑₛTₑₛ^{m}CₑₛA_{ds}G_{ds}T_{ds}^{m}C_{ds}A_{ds}T_{ds} | GalNAc₃-3a | A_{d} | 831 |
| | G_{ds}A_{ds}^{m}C_{ds}T_{ds}Tₑₛ^{m}Cₑₛ^{m}CₑₛTₑₛTₑ | | | |
| 666904 | GalNAc₃-3ₐ-_{o′}Gₑₛ^{m}CₑₛTₑₛTₑₛ^{m}CₑₛA_{ds}G_{ds}T_{ds}^{m}C_{ds}A_{ds}T_{ds} | GalNAc₃-3a | PO | 829 |
| | G_{ds}A_{ds}^{m}C_{ds}T_{ds}Tₑₛ^{m}Cₑₛ^{m}CₑₛTₑₛTₑ | | | |
| 673502 | GalNAc₃-10ₐ-_{o′}A_{do}Gₑₛ^{m}CₑₒTₑₒTₑₒ^{m}CₑₒA_{ds}G_{ds}T_{ds}^{m}C_{ds}A_{ds}T_{ds} | GalNAc₃-10a | A_{d} | 831 |
| | G_{ds}A_{ds}^{m}C_{ds}T_{ds}Tₑₒ^{m}Cₑₒ^{m}CₑₛTₑₛTₑ | | | |
| 677844 | GalNAc₃-9ₐ-_{o′}A_{do}Gₑₛ^{m}CₑₛTₑₛTₑₛ^{m}CₑₛA_{ds}G_{ds}T_{ds}^{m}C_{ds}A_{ds}T_{ds} | GalNAc₃-9a | A_{d} | 831 |
| | G_{ds}A_{ds}^{m}C_{ds}T_{ds}Tₑₛ^{m}Cₑₛ^{m}CₑₛTₑₛTₑ | | | |
| 677843 | GalNAc₃-23ₐ-_{o′}A_{do}Gₑₛ^{m}CₑₛTₑₛTₑₛ^{m}CₑₛA_{ds}G_{ds}T_{ds}^{m}C_{ds}A_{ds}T_{ds} | GalNAc₃-23a | A_{d} | 831 |
| | G_{ds}A_{ds}^{m}C_{ds}T_{ds}Tₑₛ^{m}Cₑₛ^{m}CₑₛTₑₛTₑ | | | |
| 655861 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ^{m}Cₑₛ | GalNAc₃-1a | A_{d} | 830 |
| | ^{m}CₑₛTₑₛTₑₒA_{do′}-GalNAc₃-1ₐ | | | |
| 677841 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ^{m}Cₑₛ | GalNAc₃-19a | A_{d} | 830 |
| | ^{m}CₑₛTₑₛTₑₒA_{do′}-GalNAc₃-19ₐ | | | |
| 677842 | Gₑₛ^{m}CₑₛTₑₛTₑₛ^{m}CₑₛA_{ds}G_{ds}T_{ds}^{m}C_{ds}A_{ds}T_{ds}G_{ds}A_{ds}^{m}C_{ds}T_{ds}Tₑₛ^{m}Cₑₛ | GalNAc₃-20a | A_{d} | 830 |
| | ^{m}CₑₛTₑₛTₑₒA_{do′}-GalNAc₃-20ₐ | | | |

The structure of GalNAc₃-1ₐ was shown previously in Example 9, GalNAc₃-3ₐ was shown in Example 39, GalNAc₃-9ₐ was shown in Example 52, GalNAc₃-10ₐ was shown in Example 46, GalNAc₃-19ₐ was shown in Example 70, GalNAc₃-20ₐ was shown in Example 71, and GalNAc₃-23ₐ was shown in Example 76.

### Treatment

Six to eight week old C57BL/6 mice (Jackson Laboratory, Bar Harbor, ME) were each injected subcutaneously once at a dosage shown below with an oligonucleotide listed in Table 64 or with saline. Each treatment group consisted of 4 animals. The mice were sacrificed 72 hours following the final administration to determine the SRB-1 mRNA levels using real-time PCR and RIBOGREEN® RNA quantification reagent (Molecular Probes, Inc. Eugene, OR) according to standard protocols. The results below are presented as the average percent of SRB-1 mRNA levels for each treatment group, normalized to the saline control.

As illustrated in Table 65, treatment with antisense oligonucleotides lowered SRB-1 mRNA levels in a dose-dependent manner.

TABLE 65
SRB-1 mRNA (% Saline)

| ISIS | Dosage | SRB-1 mRNA | GalNAc₃ | |
|---|---|---|---|---|
| No. | (mg/kg) | (% Saline) | Cluster | CM |
| Saline | n/a | 100.0 | n/a | n/a |
| 661161 | 0.5 | 89.18 | GalNAc₃-3a | A_{d} |
| | 1.5 | 77.02 | | |
| | 5 | 29.10 | | |
| | 15 | 12.64 | | |
| 666904 | 0.5 | 93.11 | GalNAc₃-3a | PO |
| | 1.5 | 55.85 | | |
| | 5 | 21.29 | | |
| | 15 | 13.43 | | |
| 673502 | 0.5 | 77.75 | GalNAc₃-10a | A_{d} |
| | 1.5 | 41.05 | | |
| | 5 | 19.27 | | |
| | 15 | 14.41 | | |
| 677844 | 0.5 | 87.65 | GalNAc₃-9a | A_{d} |
| | 1.5 | 93.04 | | |
| | 5 | 40.77 | | |
| | 15 | 16.95 | | |
| 677843 | 0.5 | 102.28 | GalNAc₃-23a | A_{d} |
| | 1.5 | 70.51 | | |
| | 5 | 30.68 | | |
| | 15 | 13.26 | | |
| 655861 | 0.5 | 79.72 | GalNAc₃-1a | A_{d} |
| | 1.5 | 55.48 | | |
| | 5 | 26.99 | | |
| | 15 | 17.58 | | |
| 677841 | 0.5 | 67.43 | GalNAc₃-19a | A_{d} |
| | 1.5 | 45.13 | | |
| | 5 | 27.02 | | |
| | 15 | 12.41 | | |
| 677842 | 0.5 | 64.13 | GalNAc₃-20a | A_{d} |
| | 1.5 | 53.56 | | |
| | 5 | 20.47 | | |
| | 15 | 10.23 | | |

Liver transaminase levels, alanine aminotransferase (ALT) and aspartate aminotransferase (AST), in serum were also measured using standard protocols. Total bilirubin and BUN were also evaluated. Changes in body weights were evaluated, with no significant change from the saline group (data not shown). ALTs, ASTs, total bilirubin and BUN values are shown in Table 66 below.

TABLE 66

| | | | | Total | | | |
|---|---|---|---|---|---|---|---|
| ISIS | Dosage | ALT | AST | Bilirubin | BUN | GalNAc₃ | |
| No. | (mg/kg) | (U/L) | (U/L) | (mg/dL) | (mg/dL) | Cluster | CM |
| Saline | n/a | 21 | 45 | 0.13 | 34 | n/a | n/a |
| 661161 | 0.5 | 28 | 51 | 0.14 | 39 | GalNAc₃-3a | A_{d} |
| | 1.5 | 23 | 42 | 0.13 | 39 | | |
| | 5 | 22 | 59 | 0.13 | 37 | | |
| | 15 | 21 | 56 | 0.15 | 35 | | |
| 666904 | 0.5 | 24 | 56 | 0.14 | 37 | GalNAc₃-3a | PO |
| | 1.5 | 26 | 68 | 0.15 | 35 | | |
| | 5 | 23 | 77 | 0.14 | 34 | | |
| | 15 | 24 | 60 | 0.13 | 35 | | |
| 673502 | 0.5 | 24 | 59 | 0.16 | 34 | GalNAc₃-10a | A_{d} |
| | 1.5 | 20 | 46 | 0.17 | 32 | | |
| | 5 | 24 | 45 | 0.12 | 31 | | |
| | 15 | 24 | 47 | 0.13 | 34 | | |
| 677844 | 0.5 | 25 | 61 | 0.14 | 37 | GalNAc₃-9a | A_{d} |
| | 1.5 | 23 | 64 | 0.17 | 33 | | |
| | 5 | 25 | 58 | 0.13 | 35 | | |
| | 15 | 22 | 65 | 0.14 | 34 | | |
| 677843 | 0.5 | 53 | 53 | 0.13 | 35 | GalNAc₃-23a | A_{d} |
| | 1.5 | 25 | 54 | 0.13 | 34 | | |
| | 5 | 21 | 60 | 0.15 | 34 | | |
| | 15 | 22 | 43 | 0.12 | 38 | | |
| 655861 | 0.5 | 21 | 48 | 0.15 | 33 | GalNAc₃-1a | A_{d} |
| | 1.5 | 28 | 54 | 0.12 | 35 | | |
| | 5 | 22 | 60 | 0.13 | 36 | | |
| | 15 | 21 | 55 | 0.17 | 30 | | |
| 677841 | 0.5 | 32 | 54 | 0.13 | 34 | GalNAc₃-19a | A_{d} |
| | 1.5 | 24 | 56 | 0.14 | 34 | | |
| | 5 | 23 | 92 | 0.18 | 31 | | |
| | 15 | 24 | 58 | 0.15 | 31 | | |
| 677842 | 0.5 | 23 | 61 | 0.15 | 35 | GalNAc₃-20a | A_{d} |
| | 1.5 | 24 | 57 | 0.14 | 34 | | |
| | 5 | 41 | 62 | 0.15 | 35 | | |
| | 15 | 24 | 37 | 0.14 | 32 | | |

### Example 78: Antisense Inhibition In Vivo by Oligonucleotides Targeting Angiotensinogen Comprising a GalNAc₃ Conjugate

The oligonucleotides listed below were tested in a dose-dependent study for antisense inhibition of Angiotensinogen (AGT) in normotensive Sprague Dawley rats.

TABLE 67
Modified ASOs targeting AGT

| ISIS | Sequences | GalNAc₃ | | SEQ |
|---|---|---|---|---|
| No. | (5′ to 3′) | Cluster | CM | ID No. |
| 552668 | ^{m}CₑₛAₑₛ^{m}CₑₛTₑₛGₑₛ | n/a | n/a | 835 |
| | A_{ds}T_{ds}T_{ds}T_{ds}T_{ds} | | | |
| | T_{ds}G_{ds}^{m}C_{ds}^{m}C_{ds}^{m}C_{ds} | | | |
| | AₑₛGₑₛGₑₛAₑₛTₑ | | | |
| 669509 | ^{m}CₑₛAₑₛ^{m}CₑₛTₑₛGₑₛA_{ds} | GalNAc₃- | A_{d} | 836 |
| | T_{ds}T_{ds}T_{ds}T_{ds}T_{ds}G_{ds}^{m}C_{ds} | 1ₐ | | |
| | ^{m}C_{ds}^{m}C_{ds}AₑₛGₑₛGₑₛAₑₛ | | | |
| | TₑₒA_{do′}-GalNAc₃-1ₐ | | | |

The structure of GalNAc₃-1ₐ was shown previously in Example 9.

### Treatment

Six week old, male Sprague Dawley rats were each injected subcutaneously once per week at a dosage shown below, for a total of three doses, with an oligonucleotide listed in Table 67 or with PBS. Each treatment group consisted of 4 animals. The rats were sacrificed 72 hours following the final dose. AGT liver mRNA levels were measured using real-time PCR and RIBOGREEN® RNA quantification reagent (Molecular Probes, Inc. Eugene, OR) according to standard protocols. AGT plasma protein levels were measured using the Total Angiotensinogen ELISA (Catalog #JP27412, IBL International, Toronto, ON) with plasma diluted 1:20,000. The results below are presented as the average percent of AGT mRNA levels in liver or AGT protein levels in plasma for each treatment group, normalized to the PBS control.

As illustrated in Table 68, treatment with antisense oligonucleotides lowered AGT liver mRNA and plasma protein levels in a dose-dependent manner, and the oligonucleotide comprising a GalNAc conjugate was significantly more potent than the parent oligonucleotide lacking a GalNAc conjugate.

TABLE 68
AGT liver mRNA and plasma protein levels

| | | AGT | AGT | | |
|---|---|---|---|---|---|
| | | liver | plasma | | |
| ISIS | Dosage | mRNA | protein | GalNAc₃ | |
| No. | (mg/kg) | (% PBS) | (% PBS) | Cluster | CM |
| PBS | n/a | 100 | 100 | n/a | n/a |
| 552668 | 3 | 95 | 122 | n/a | n/a |
| | 10 | 85 | 97 | | |
| | 30 | 46 | 79 | | |
| | 90 | 8 | 11 | | |
| 669509 | 0.3 | 95 | 70 | GalNAc₃-1a | A_{d} |
| | 1 | 95 | 129 | | |
| | 3 | 62 | 97 | | |
| | 10 | 9 | 23 | | |

Liver transaminase levels, alanine aminotransferase (ALT) and aspartate aminotransferase (AST), in plasma and body weights were also measured at time of sacrifice using standard protocols.

The results are shown in Table 69 below.

TABLE 69
Liver transaminase levels and rat body weights

| | | | | Body Weight | | |
|---|---|---|---|---|---|---|
| ISIS | Dosage | ALT | AST | (% of | GalNAc₃ | |
| No. | (mg/kg) | (U/L) | (U/L) | baseline) | Cluster | CM |
| PBS | n/a | 51 | 81 | 186 | n/a | n/a |
| 552668 | 3 | 54 | 93 | 183 | n/a | n/a |
| | 10 | 51 | 93 | 194 | | |
| | 30 | 59 | 99 | 182 | | |
| | 90 | 56 | 78 | 170 | | |
| 669509 | 0.3 | 53 | 90 | 190 | GalNAc₃-1a | A_{d} |
| | 1 | 51 | 93 | 192 | | |
| | 3 | 48 | 85 | 189 | | |
| | 10 | 56 | 95 | 189 | | |

### Example 79: Duration of Action In Vivo of Oligonucleotides Targeting APOC-II Comprising a GalNAc₃ Conjugate

The oligonucleotides listed in Table 70 below were tested in a single dose study for duration of action in mice.

TABLE 70
Modified ASOs targeting APOC-III

| ISIS | Sequences | GalNAc₃ | | SEQ |
|---|---|---|---|---|
| No. | (5′ to 3′) | Cluster | CM | ID No. |
| 304801 | AₑₛGₑₛ^{m}CₑₛTₑₛTₑₛ | n/a | n/a | 821 |
| | ^{m}C_{ds}T_{ds}T_{ds}G_{ds}T_{ds} | | | |
| | ^{m}C_{ds}^{m}C_{ds}A_{ds}G_{ds}^{m}C_{ds} | | | |
| | TₑₛTₑₛTₑₛAₑₛTₑ | | | |
| 647535 | AₑₛGₑₛ^{m}CₑₛTₑₛTₑₛ^{m}C_{ds} | GalNAc₃- | A_{d} | 822 |
| | T_{ds}T_{ds}G_{ds}T_{ds}^{m}C_{ds}^{m}C_{ds} | 1a | | |
| | A_{ds}G_{ds}^{m}C_{ds}TₑₛTₑₛTₑₛAₑₛ | | | |
| | TₑₒA_{do′}-GalNAc₃-1ₐ | | | |
| 663083 | GalNAc₃-3ₐ-_{o′}A_{do}Aₑₛ | GalNAc₃- | A_{d} | 837 |
| | Gₑₛ^{m}CₑₛTₑₛTₑₛ^{m}C_{ds}T_{ds} | 3a | | |
| | T_{ds}G_{ds}T_{ds}^{m}C_{ds}^{m}C_{ds}A_{ds} | | | |
| | G_{ds}^{m}C_{ds}TₑₛTₑₛTₑₛAₑₛTₑ | | | |
| 674449 | GalNAc₃-7ₐ-_{o′}A_{do}Aₑₛ | GalNAc₃- | A_{d} | 837 |
| | Gₑₛ^{m}CₑₛTₑₛTₑₛ^{m}C_{ds}T_{ds} | 7a | | |
| | T_{ds}G_{ds}T_{ds}^{m}C_{ds}^{m}C_{ds}A_{ds} | | | |
| | G_{ds}^{m}C_{ds}TₑₛTₑₛTₑₛAₑₛTₑ | | | |
| 674450 | GalNAc₃-10ₐ-_{o′}A_{do}Aₑₛ | GalNAc₃- | A_{d} | 837 |
| | Gₑₛ^{m}CₑₛTₑₛTₑₛ^{m}C_{ds}T_{ds} | 10a | | |
| | T_{ds}G_{ds}T_{ds}^{m}C_{ds}^{m}C_{ds}A_{ds} | | | |
| | G_{ds}^{m}C_{ds}TₑₛTₑₛTₑₛAₑₛTₑ | | | |
| 674451 | GalNAc₃-13ₐ-_{o′}A_{do}Aₑₛ | GalNAc₃- | A_{d} | 837 |
| | Gₑₛ^{m}CₑₛTₑₛTₑₛ^{m}C_{ds}T_{ds} | 13a | | |
| | T_{ds}G_{ds}T_{ds}^{m}C_{ds}^{m}C_{ds}A_{ds} | | | |
| | G_{ds}^{m}C_{ds}TₑₛTₑₛTₑₛAₑₛTₑ | | | |

The structure of GalNAc₃-1ₐ was shown previously in Example 9, GalNAc₃-3ₐ was shown in Example 39, GalNAc₃-7ₐ was shown in Example 48, GalNAc₃-10ₐ was shown in Example 46, and GalNAc₃-13ₐ was shown in Example 62.

### Treatment

Six to eight week old transgenic mice that express human APOC-III were each injected subcutaneously once with an oligonucleotide listed in Table 70 or with PBS. Each treatment group consisted of 3 animals. Blood was drawn before dosing to determine baseline and at 72 hours, 1 week, 2 weeks, 3 weeks, 4 weeks, 5 weeks, and 6 weeks following the dose. Plasma triglyceride and APOC-III protein levels were measured as described in Example 20. The results below are presented as the average percent of plasma triglyceride and APOC-II levels for each treatment group, normalized to baseline levels, showing that the oligonucleotides comprising a GalNAc conjugate group exhibited a longer duration of action than the parent oligonucleotide without a conjugate group (ISIS 304801) even though the dosage of the parent was three times the dosage of the oligonucleotides comprising a GalNAc conjugate group.

TABLE 71
Plasma triglyceride and APOC-III protein levels in transgenic mice

| | | Time point | | | | |
|---|---|---|---|---|---|---|
| ISIS | Dosage | (days post- | Triglycerides | APOC-III protein | GalNAc₃ | |
| No. | (mg/kg) | dose) | (% baseline) | (% baseline) | Cluster | CM |
| PBS | n/a | 3 | 97 | 102 | n/a | n/a |
| | | 7 | 101 | 98 | | |
| | | 14 | 108 | 98 | | |
| | | 21 | 107 | 107 | | |
| | | 28 | 94 | 91 | | |
| | | 35 | 88 | 90 | | |
| | | 42 | 91 | 105 | | |
| 304801 | 30 | 3 | 40 | 34 | n/a | n/a |
| | | 7 | 41 | 37 | | |
| | | 14 | 50 | 57 | | |
| | | 21 | 50 | 50 | | |
| | | 28 | 57 | 73 | | |
| | | 35 | 68 | 70 | | |
| | | 42 | 75 | 93 | | |
| 647535 | 10 | 3 | 36 | 37 | GalNAc₃-1a | A_{d} |
| | | 7 | 39 | 47 | | |
| | | 14 | 40 | 45 | | |
| | | 21 | 41 | 41 | | |
| | | 28 | 42 | 62 | | |
| | | 35 | 69 | 69 | | |
| | | 42 | 85 | 102 | | |
| 663083 | 10 | 3 | 24 | 18 | GalNAc₃-3a | A_{d} |
| | | 7 | 28 | 23 | | |
| | | 14 | 25 | 27 | | |
| | | 21 | 28 | 28 | | |
| | | 28 | 37 | 44 | | |
| | | 35 | 55 | 57 | | |
| | | 42 | 60 | 78 | | |
| 674449 | 10 | 3 | 29 | 26 | GalNAc₃-7a | A_{d} |
| | | 7 | 32 | 31 | | |
| | | 14 | 38 | 41 | | |
| | | 21 | 44 | 44 | | |
| | | 28 | 53 | 63 | | |
| | | 35 | 69 | 77 | | |
| | | 42 | 78 | 99 | | |
| 674450 | 10 | 3 | 33 | 30 | GalNAc₃-10a | A_{d} |
| | | 7 | 35 | 34 | | |
| | | 14 | 31 | 34 | | |
| | | 21 | 44 | 44 | | |
| | | 28 | 56 | 61 | | |
| | | 35 | 68 | 70 | | |
| | | 42 | 83 | 95 | | |
| 674451 | 10 | 3 | 35 | 33 | GalNAc₃-13a | A_{d} |
| | | 7 | 24 | 32 | | |
| | | 14 | 40 | 34 | | |
| | | 21 | 48 | 48 | | |
| | | 28 | 54 | 67 | | |
| | | 35 | 65 | 75 | | |
| | | 42 | 74 | 97 | | |

### Example 80: Antisense Inhibition In Vivo by Oligonucleotides Targeting Alpha-1 Antitrypsin (A1AT) Comprising a GalNAc₃ Conjugate

The oligonucleotides listed in Table 72 below were tested in a study for dose-dependent inhibition of A1AT in mice.

TABLE 72
Modified ASOs targeting A1AT

| ISIS | Sequences | GalNAc₃ | | SEQ |
|---|---|---|---|---|
| No. | (5′ to 3′) | Cluster | CM | ID No. |
| 476366 | Aₑₛ^{m}Cₑₛ^{m}Cₑₛ^{m}CₑₛAₑₛ | n/a | n/a | 838 |
| | A_{ds}T_{ds}T_{ds}^{m}C_{ds}A_{ds} | | | |
| | G_{ds}A_{ds}A_{ds}G_{ds}G_{ds} | | | |
| | AₑₛAₑₛGₑₛGₑₛAₑ | | | |
| 656326 | Aₑₛ^{m}Cₑₛ^{m}Cₑₛ^{m}CₑₛAₑₛA_{ds} | GalNAc₃- | A_{d} | 839 |
| | T_{ds}T_{ds}^{m}C_{ds}A_{ds}G_{ds}A_{ds} | 1a | | |
| | A_{ds}G_{ds}G_{ds}AₑₛAₑₛGₑₛGₑₛ | | | |
| | AₑₒA_{do′}-GalNAc₃-1ₐ | | | |
| 678381 | GalNAc₃-3ₐ-_{o′}A_{do}Aₑₛ | GalNAc₃- | A_{d} | 840 |
| | ^{m}Cₑₛ^{m}Cₑₛ^{m}CₑₛAₑₛA_{ds}T_{ds} | 3a | | |
| | T_{ds}^{m}C_{ds}A_{ds}G_{ds}A_{ds}A_{ds} | | | |
| | G_{ds}G_{ds}AₑₛAₑₛGₑₛGₑₛAₑ | | | |
| 678382 | GalNAc₃-7ₐ-_{o′}A_{do}Aₑₛ | GalNAc₃- | A_{d} | 840 |
| | ^{m}Cₑₛ^{m}Cₑₛ^{m}CₑₛAₑₛA_{ds}T_{ds} | 7a | | |
| | T_{ds}^{m}C_{ds}A_{ds}G_{ds}A_{ds}A_{ds}G_{ds} | | | |
| | G_{ds}AₑₛAₑₛGₑₛGₑₛAₑ | | | |
| 678383 | GalNAc₃-10ₐ-_{o′}A_{do} | GalNAc₃- | A_{d} | 840 |
| | Aₑₛ^{m}Cₑₛ^{m}Cₑₛ^{m}CₑₛAₑₛA_{ds} | 10a | | |
| | T_{ds}T_{ds}^{m}C_{ds}A_{ds}G_{ds}A_{ds}A_{ds} | | | |
| | G_{ds}G_{ds}AₑₛAₑₛGₑₛGₑₛAₑ | | | |
| 678384 | GalNAc₃-13ₐ-_{o′}A_{do} | GalNAc₃- | A_{d} | 840 |
| | Aₑₛ^{m}Cₑₛ^{m}Cₑₛ^{m}CₑₛAₑₛA_{ds} | 13a | | |
| | T_{ds}T_{ds}^{m}C_{ds}A_{ds}G_{ds}A_{ds}A_{ds} | | | |
| | G_{ds}G_{ds}AₑₛAₑₛGₑₛGₑₛAₑ | | | |

The structure of GalNAc₃-1ₐ was shown previously in Example 9, GalNAc₃-3ₐ was shown in Example 39, GalNAc₃-7ₐ was shown in Example 48, GalNAc₃-10ₐ was shown in Example 46, and GalNAc₃-13ₐ was shown in Example 62.

### Treatment

Six week old, male C57BL/6 mice (Jackson Laboratory, Bar Harbor, ME) were each injected subcutaneously once per week at a dosage shown below, for a total of three doses, with an oligonucleotide listed in Table 72 or with PBS. Each treatment group consisted of 4 animals. The mice were sacrificed 72 hours following the final administration. AAT liver mRNA levels were determined using real-time PCR and RIBOGREEN® RNA quantification reagent (Molecular Probes, Inc. Eugene, OR) according to standard protocols. AAT plasma protein levels were determined using the Mouse Alpha 1-Antitrypsin ELISA (catalog #41-A1AMS-E01, Alpco, Salem, Nil). The results below are presented as the average percent of A1AT liver mRNA and plasma protein levels for each treatment group, normalized to the PBS control.

As illustrated in Table 73, treatment with antisense oligonucleotides lowered A1AT liver mRNA and A1AT plasma protein levels in a dose-dependent manner. The oligonucleotides comprising a GalNAc conjugate were significantly more potent than the parent (ISIS 476366).

TABLE 73
A1AT liver mRNA and plasma protein levels

| | | A1AT | A1AT | | |
|---|---|---|---|---|---|
| | | liver | plasma | | |
| ISIS | Dosage | mRNA | protein | GalNAc₃ | |
| No. | (mg/kg) | (% PBS) | (% PBS) | Cluster | CM |
| PBS | n/a | 100 | 100 | n/a | n/a |
| 476366 | 5 | 86 | 78 | n/a | n/a |
| | 15 | 73 | 61 | | |
| | 45 | 30 | 38 | | |
| 656326 | 0.6 | 99 | 90 | GalNAc₃-1a | A_{d} |
| | 2 | 61 | 70 | | |
| | 6 | 15 | 30 | | |
| | 18 | 6 | 10 | | |
| 678381 | 0.6 | 105 | 90 | GalNAc₃-3a | A_{d} |
| | 2 | 53 | 60 | | |
| | 6 | 16 | 20 | | |
| | 18 | 7 | 13 | | |
| 678382 | 0.6 | 90 | 79 | GalNAc₃-7a | A_{d} |
| | 2 | 49 | 57 | | |
| | 6 | 21 | 27 | | |
| | 18 | 8 | 11 | | |
| 678383 | 0.6 | 94 | 84 | GalNAc₃-10a | A_{d} |
| | 2 | 44 | 53 | | |
| | 6 | 13 | 24 | | |
| | 18 | 6 | 10 | | |
| 678384 | 0.6 | 106 | 91 | GalNAc₃-13a | A_{d} |
| | 2 | 65 | 59 | | |
| | 6 | 26 | 31 | | |
| | 18 | 11 | 15 | | |

Liver transaminase and BUN levels in plasma were measured at time of sacrifice using standard protocols. Body weights and organ weights were also measured. The results are shown in Table 74 below. Body weight is shown as 00 relative to baseline. Organ weights are shown as 0% of body weight relative to the PBS control group.

TABLE 74

| ISIS | Dosage | ALT | AST | BUN | Body weight | Liver weight | Kidney weight | Spleen weight |
|---|---|---|---|---|---|---|---|---|
| No. | (mg/kg) | (U/L) | (U/L) | (mg/dL) | (% baseline) | (Rel % BW) | (Rel % BW) | (Rel % BW) |
| PBS | n/a | 25 | 51 | 37 | 119 | 100 | 100 | 100 |
| 476366 | 5 | 34 | 68 | 35 | 116 | 91 | 98 | 106 |
| | 15 | 37 | 74 | 30 | 122 | 92 | 101 | 128 |
| | 45 | 30 | 47 | 31 | 118 | 99 | 108 | 123 |
| 656326 | 0.6 | 29 | 57 | 40 | 123 | 100 | 103 | 119 |
| | 2 | 36 | 75 | 39 | 114 | 98 | 111 | 106 |
| | 6 | 32 | 67 | 39 | 125 | 99 | 97 | 122 |
| | 18 | 46 | 77 | 36 | 116 | 102 | 109 | 101 |
| 678381 | 0.6 | 26 | 57 | 32 | 117 | 93 | 109 | 110 |
| | 2 | 26 | 52 | 33 | 121 | 96 | 106 | 125 |
| | 6 | 40 | 78 | 32 | 124 | 92 | 106 | 126 |
| | 18 | 31 | 54 | 28 | 118 | 94 | 103 | 120 |
| 678382 | 0.6 | 26 | 42 | 35 | 114 | 100 | 103 | 103 |
| | 2 | 25 | 50 | 31 | 117 | 91 | 104 | 117 |
| | 6 | 30 | 79 | 29 | 117 | 89 | 102 | 107 |
| | 18 | 65 | 112 | 31 | 120 | 89 | 104 | 113 |
| 678383 | 0.6 | 30 | 67 | 38 | 121 | 91 | 100 | 123 |
| | 2 | 33 | 53 | 33 | 118 | 98 | 102 | 121 |
| | 6 | 32 | 63 | 32 | 117 | 97 | 105 | 105 |
| | 18 | 36 | 68 | 31 | 118 | 99 | 103 | 108 |
| 678384 | 0.6 | 36 | 63 | 31 | 118 | 98 | 103 | 98 |
| | 2 | 32 | 61 | 32 | 119 | 93 | 102 | 114 |
| | 6 | 34 | 69 | 34 | 122 | 100 | 100 | 96 |
| | 18 | 28 | 54 | 30 | 117 | 98 | 101 | 104 |

### Example 81: Duration of Action In Vivo of Oligonucleotides Targeting AMAT Comprising a GalNAc₃ Cluster

The oligonucleotides listed in Table 72 were tested in a single dose study for duration of action in mice.

### Treatment

Six week old, male C57BL/6 mice were each injected subcutaneously once with an oligonucleotide listed in Table 72 or with PBS. Each treatment group consisted of 4 animals. Blood was drawn the day before dosing to determine baseline and at 5, 12, 19, and 25 days following the dose. Plasma AMAT protein levels were measured via ELISA (see Example 80). The results below are presented as the average percent of plasma AMAT protein levels for each treatment group, normalized to baseline levels. The results show that the oligonucleotides comprising a GalNAc conjugate were more potent and had longer duration of action than the parent lacking a GalNAc conjugate (ISIS 476366). Furthermore, the oligonucleotides comprising a 5′-GalNAc conjugate (ISIS 678381, 678382, 678383, and 678384) were generally even more potent with even longer duration of action than the oligonucleotide comprising a 3′-GalNAc conjugate (ISIS 656326).

TABLE 75
Plasma A1AT protein levels in mice

| | | Time point | | | |
|---|---|---|---|---|---|
| ISIS | Dosage | (days post- | A1AT | GalNAc₃ | |
| No. | (mg/kg) | dose) | (% baseline) | Cluster | CM |
| PBS | n/a | 5 | 93 | n/a | n/a |
| | | 12 | 93 | | |
| | | 19 | 90 | | |
| | | 25 | 97 | | |
| 476366 | 100 | 5 | 38 | n/a | n/a |
| | | 12 | 46 | | |
| | | 19 | 62 | | |
| | | 25 | 77 | | |
| 656326 | 18 | 5 | 33 | GalNAc₃-1a | A_{d} |
| | | 12 | 36 | | |
| | | 19 | 51 | | |
| | | 25 | 72 | | |
| 678381 | 18 | 5 | 21 | GalNAc₃-3a | A_{d} |
| | | 12 | 21 | | |
| | | 19 | 35 | | |
| | | 25 | 48 | | |
| 678382 | 18 | 5 | 21 | GalNAc₃-7a | A_{d} |
| | | 12 | 21 | | |
| | | 19 | 39 | | |
| | | 25 | 60 | | |
| 678383 | 18 | 5 | 24 | GalNAc₃-10a | A_{d} |
| | | 12 | 21 | | |
| | | 19 | 45 | | |
| | | 25 | 73 | | |
| 678384 | 18 | 5 | 29 | GalNAc₃-13a | A_{d} |
| | | 12 | 34 | | |
| | | 19 | 57 | | |
| | | 25 | 76 | | |

### Example 82: Antisense Inhibition In Vitro by Oligonucleotides Targeting SRB-1 Comprising a GalNAc₃ Conjugate

Primary mouse liver hepatocytes were seeded in 96 well plates at 15,000 cells/well 2 hours prior to treatment. The oligonucleotides listed in Table 76 were added at 2, 10, 50, or 250 nM in Williams E medium and cells were incubated overnight at 37° C. in 500 C02. Cells were lysed 16 hours following oligonucleotide addition, and total RNA was purified using RNease 3000 BioRobot (Qiagen). SRB-1 mRNA levels were determined using real-time PCR and RIBOGREEN® RNA quantification reagent (Molecular Probes, Inc. Eugene, OR) according to standard protocols. IC₅₀ values were determined using Prism 4 software (GraphPad). The results show that oligonucleotides comprising a variety of different GalNAc conjugate groups and a variety of different cleavable moieties are significantly more potent in an in vitro free uptake experiment than the parent oligonucleotides lacking a GalNAc conjugate group (ISIS 353382 and 666841).

TABLE 76
Inhibition of SRB-1 expression in vitro

| ISIS | | | GalNAc | | IC₅₀ | SEQ |
|---|---|---|---|---|---|---|
| No. | Sequence (5′ to 3′) | Linkages | cluster | CM | (nM) | ID No. |
| 353382 | Gₑₛ^{m}CₑₛTₑₛTₑₛ^{m}CₑₛA_{ds}G_{ds}T_{ds}^{m}C_{ds}A_{ds} | PS | n/a | n/a | 250 | 829 |
| | T_{ds}G_{ds}A_{ds}^{m}C_{ds}T_{ds}Tₑₛ^{m}Cₑₛ^{m}CₑₛTₑₛTₑ | | | | | |
| 655861 | Gₑₛ^{m}CₑₛTₑₛTₑₛ^{m}CₑₛA_{ds}G_{ds}T_{ds}^{m}C_{ds}A_{ds} | PS | GalNAc₃-1ₐ | A_{d} | 40 | 830 |
| | T_{ds}G_{ds}A_{ds}^{m}C_{ds}T_{ds}Tₑₛ^{m}Cₑₛ^{m}CₑₛTₑₛTₑₒ | | | | | |
| | A_{do′}-GalNAc₃-1ₐ | | | | | |
| 661161 | GalNAc₃-3ₐ-_{o′}A_{do}Gₑₛ^{m}CₑₛTₑₛ | PS | GalNAc₃-3ₐ | A_{d} | 40 | 831 |
| | Tₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 661162 | GalNAc₃-3ₐ-_{o′}A_{do}Gₑₛ^{m}CₑₒTₑₒ | PO/PS | GalNAc₃-3ₐ | A_{d} | 8 | 831 |
| | Tₑₒ^{m}TₑₒA_{ds}G_{ds}T_{ds}^{m}C_{ds}A_{ds}T_{ds}G_{ds} | | | | | |
| | A_{ds}^{m}C_{ds}T_{ds}Tₑₒ^{m}Cₑₒ^{m}CₑₛTₑₛTₑ | | | | | |
| 664078 | Gₑₛ^{m}CₑₛTₑₛTₑₛ^{m}CₑₛA_{ds}G_{ds}T_{ds}^{m}C_{ds} | PS | GalNAc₃-9ₐ | A_{d} | 20 | 830 |
| | A_{ds}T_{ds}G_{ds}A_{ds}^{m}C_{ds}T_{ds}Tₑₛ^{m}Cₑₛ^{m}Cₑₛ | | | | | |
| | TₑₛTₑₒA_{do′}-GalNAc₃-9ₐ | | | | | |
| 665001 | GalNAc₃-8ₐ-_{o′}A_{do}Gₑₛ^{m}CₑₛTₑₛ | PS | GalNAc₃-8ₐ | A_{d} | 70 | 831 |
| | Tₑₛ^{m}CₑₛA_{ds}G_{ds}T_{ds}^{m}C_{ds}-A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 666224 | GalNAc₃-5ₐ-_{o′}A_{do}Gₑₛ^{m}CₑₛTₑₛ | PS | GalNAc₃-5ₐ | A_{d} | 80 | 831 |
| | Tₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}cₑₛ^{m}cₑₛTₑₛTₑ | | | | | |
| 666841 | Gₑₛ^{m}CₑₒTₑₒTₑₒ^{m}CₑₛA_{ds}G_{ds}T_{ds}^{m}C_{ds}A_{ds} | PO/PS | n/a | n/a | >250 | 829 |
| | T_{ds}G_{ds}A_{ds}^{m}C_{ds}T_{ds}Tₑₒ^{m}Cₑₒ^{m}CₑₛTₑₛTₑ | | | | | |
| 666881 | GalNAc₃-10ₐ-_{o′}A_{do}Gₑₛ^{m}Cₑₛ | PS | GalNAc₃-10ₐ | A_{d} | 30 | 831 |
| | TₑₛTₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 666904 | GalNAc₃-3ₐ-_{o′}Gₑₛ^{m}CₑₛTₑₛTₑₛ | PS | GalNAc₃-3ₐ | PO | 9 | 829 |
| | ^{m}CₑₛA_{ds}G_{ds}T_{ds}^{m}C_{ds}A_{ds}T_{ds}G_{ds} | | | | | |
| | A_{ds}^{m}C_{ds}T_{ds}Tₑₛ^{m}Cₑₛ^{m}CₑₛTₑₛTₑ | | | | | |
| 666924 | GalNAc₃-3ₐ-_{o′}T_{do}Gₑₛ^{m}CₑₛTₑₛ | PS | GalNAc₃-3ₐ | T_{d} | 15 | 834 |
| | Tₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 666961 | GalNAc₃-6ₐ-_{o′}A_{do}Gₑₛ^{m}CₑₛTₑₛ | PS | GalNAc₃-6ₐ | A_{d} | 150 | 831 |
| | Tₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 666981 | GalNAc₃-7ₐ-_{o′}A_{do}Gₑₛ^{m}CₑₛTₑₛ | PS | GalNAc₃-7ₐ | A_{d} | 20 | 831 |
| | Tₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 670061 | GalNAc₃-13ₐ-_{o′}A_{do}Gₑₛ^{m}CₑₛTₑₛ | PS | GalNAc₃-13ₐ | A_{d} | 30 | 831 |
| | Tₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 670699 | GalNAc₃-3ₐ-_{o′}T_{do}Gₑₛ^{m}CₑₒTₑₒ | PO/PS | GalNAc₃-3ₐ | T_{d} | 15 | 834 |
| | Tₑₒ^{m}TₑₒA_{ds}G_{ds}T_{ds}^{m}C_{ds}A_{ds}T_{ds} | | | | | |
| | G_{ds}A_{ds}^{m}C_{ds}T_{ds}Tₑₒ^{m}Cₑₒ^{m}CₑₛTₑₛTₑ | | | | | |
| 670700 | GalNAc₃-3ₐ-_{o′}AₑₒGₑₛ^{m}CₑₒTₑₒ | PO/PS | GalNAc₃-3ₐ | Aₑ | 30 | 831 |
| | Tₑₒ^{m}CₑₒA_{ds}G_{ds}T_{ds}^{m}C_{ds}A_{ds}T_{ds} | | | | | |
| | G_{ds}A_{ds}^{m}C_{ds}T_{ds}Tₑₒ^{m}Cₑₒ^{m}CₑₛTₑₛT | | | | | |
| 670701 | GalNAc₃-3ₐ-_{o′}TₑₒGₑₛ^{m}CₑₒTₑₒ | PO/PS | GalNAc₃-3ₐ | Tₑ | 25 | 834 |
| | Tₑₒ^{m}CₑₒA_{ds}G_{ds}T_{ds}^{m}C_{ds}A_{ds}T_{ds} | | | | | |
| | G_{ds}A_{ds}^{m}C_{ds}T_{ds}Tₑₒ^{m}Cₑₒ^{m}CₑₛTₑₛTₑ | | | | | |
| 671144 | GalNAc₃-12ₐ-_{o′}A_{do}Gₑₛ^{m}CₑₛTₑₛ | PS | GalNAc₃-12ₐ | A_{d} | 40 | 831 |
| | Tₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 671165 | GalNAc₃-13ₐ-_{o′}A_{do}Gₑₛ^{m}Cₑₒ | PO/PS | GalNAc₃-13ₐ | A_{d} | 8 | 831 |
| | TₑₒTₑₒ^{m}TₑₒA_{ds}G_{ds}T_{ds}^{m}C_{ds}A_{ds}T_{ds} | | | | | |
| | G_{ds}A_{ds}^{m}C_{ds}T_{ds}Tₑₒ^{m}Cₑₒ^{m}CₑₛTₑₛT | | | | | |
| 671261 | GalNAc₃-14ₐ-_{o′}A_{do}Gₑₛ^{m}Cₑₛ | PS | GalNAc₃-14ₐ | A_{d} | >250 | 831 |
| | TₑₛTₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 671262 | GalNAc₃-15ₐ-_{o′}A_{do}Gₑₛ^{m}Cₑₛ | PS | GalNAc₃-15ₐ | A_{d} | >250 | 831 |
| | TₑₛTₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 673501 | GalNAc₃-7ₐ-_{o′}A_{do}Gₑₛ^{m}Cₑₒ | PO/PS | GalNAc₃-7ₐ | A_{d} | 30 | 831 |
| | TₑₒTₑₒ^{m}TₑₒA_{ds}G_{ds}T_{ds}^{m}C_{ds}A_{ds}T_{ds} | | | | | |
| | G_{ds}A_{ds}^{m}C_{ds}T_{ds}Tₑₒ^{m}Cₑₒ^{m}CₑₛTₑₛTₑ | | | | | |
| 673502 | GalNAc₃-10ₐ-_{o′}A_{do}Gₑₛ^{m}Cₑₒ | PO/PS | GalNAc₃-10ₐ | A_{d} | 8 | 831 |
| | TₑₒTₑₒ^{m}CₑₒA_{ds}G_{ds}T_{ds}^{m}C_{ds}A_{ds}T_{ds} | | | | | |
| | G_{ds}A_{ds}^{m}C_{ds}T_{ds}Tₑₒ^{m}Cₑₒ^{m}CₑₛTₑₛTₑ | | | | | |
| 675441 | GalNAc₃-17ₐ-_{o′}A_{do}Gₑₛ^{m}Cₑₛ | PS | GalNAc₃-17ₐ | A_{d} | 30 | 831 |
| | TₑₛTₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 675442 | GalNAc₃-18ₐ-_{o′}A_{do}Gₑₛ^{m}Cₑₛ | PS | GalNAc₃-18ₐ | A_{d} | 20 | 831 |
| | TₑₛTₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |
| 677841 | Gₑₛ^{m}CₑₛTₑₛTₑₛ^{m}CₑₛA_{ds}G_{ds}T_{ds}^{m}C_{ds} | PS | GalNAc₃-19ₐ | A_{d} | 40 | 830 |
| | A_{ds}T_{ds}G_{ds}A_{ds}^{m}C_{ds}T_{ds}Tₑₛ^{m}Cₑₛ^{m}Cₑₛ | | | | | |
| | TₑₛTₑₒA_{do′}-GalNAc₃-19ₐ | | | | | |
| 677842 | Gₑₛ^{m}CₑₛTₑₛTₑₛ^{m}CₑₛA_{ds}G_{ds}T_{ds}^{m}C_{ds} | PS | GalNAc₃-20ₐ | A_{d} | 30 | 830 |
| | A_{ds}T_{ds}G_{ds}A_{ds}^{m}C_{ds}T_{ds}Tₑₛ^{m}Cₑₛ^{m}Cₑₛ | | | | | |
| | TₑₛTₑₒA_{do′}-GalNAc₃-20ₐ | | | | | |
| 677843 | GalNAc₃-23ₐ-_{o′}A_{do}Gₑₛ^{m}Cₑₛ | PS | GalNAc₃-23ₐ | A_{d} | 40 | 831 |
| | TₑₛTₑₛ^{m}CₑₛA_{ds}G_{ds}T_{ds}^{m}C_{ds}A_{ds}T_{ds} | | | | | |
| | G_{ds}A_{ds}^{m}C_{ds}T_{ds}Tₑₛ^{m}Cₑₛ^{m}CₑₛTₑₛTₑ | | | | | |

The structure of GalNAc3-1ₐ was shown previously in Example 9, GalNAc3-3ₐ was shown in Example 39, GalNAc3-5a was shown in Example 49, GalNAc3-6a was shown in Example 51, GalNAc3-7a was shown in Example 48, GalNAc3-8a was shown in Example 47, GalNAc3-9a was shown in Example 52, GalNAc₃-10ₐ was shown in Example 46, GalNAc₃-12a was shown in Example 61, GalNAc₃-13a was shown in Example 62, GalNAc₃-14a was shown in Example 63, GalNAc₃-15a was shown in Example 64, GalNAc₃-17a was shown in Example 68, GalNAc₃-18a was shown in Example 69, GalNAc₃-19ₐ was shown in Example 70, GalNAc₃-20ₐ was shown in Example 71, and GalNAc₃-23ₐ was shown in Example 76.

### Example 83: Antisense Inhibition In Vivo by Oligonucleotides Targeting Factor XI Comprising a GalNAc₃ Cluster

The oligonucleotides listed in Table 77 below were tested in a study for dose-dependent inhibition of Factor XI in mice.

TABLE 77
Modified oligonucleotides targeting Factor XI

| ISIS | Sequence | GalNAc | | SEQ |
|---|---|---|---|---|
| No. | (5′ to 3′) | cluster | CM | ID No. |
| 404071 | TₑₛGₑₛGₑₛTₑₛAₑₛA_{ds} | n/a | n/a | 832 |
| | T_{ds}^{m}C_{ds}^{m}C_{ds}A_{ds} | | | |
| | ^{m}C_{ds}T_{ds}T_{ds}T_{ds}^{m}C_{ds} | | | |
| | AₑₛGₑₛAₑₛGₑₛGₑ | | | |
| 656173 | TₑₛGₑₛGₑₒTₑₒAₑₒA_{ds} | GalNAc₃- | A_{d} | 833 |
| | T_{ds}^{m}C_{ds}^{m}C_{ds}A_{ds}^{m}C_{ds} | 1ₐ | | |
| | T_{ds}T_{ds}T_{ds}^{m}C_{ds}Aₑₒ | | | |
| | GₑₒAₑₛGₑₛGₑₒA_{do′}- | | | |
| | GalNAc₃-1ₐ | | | |
| 663086 | GalNAc₃-3ₐ-_{o′}A_{do} | GalNAc₃- | A_{d} | 841 |
| | TₑₛGₑₒGₑₒTₑₒAₑₒA_{ds} | 3ₐ | | |
| | T_{ds}^{m}C_{ds}^{m}C_{ds}A_{ds}^{m}C_{ds} | | | |
| | T_{ds}T_{ds}T_{ds}^{m}C_{ds}Aₑₒ | | | |
| | GₑₒAₑₛGₑₛGₑ | | | |
| 678347 | GalNAc₃-7ₐ-_{o′}A_{do} | GalNAc₃- | A_{d} | 841 |
| | TₑₛGₑₒGₑₒTₑₒAₑₒA_{ds} | 7ₐ | | |
| | T_{ds}^{m}C_{ds}^{m}C_{ds}A_{ds}^{m}C_{ds} | | | |
| | T_{ds}T_{ds}T_{ds}^{m}C_{ds}Aₑₒ | | | |
| | GₑₒAₑₛGₑₛGₑ | | | |
| 678348 | GalNAc₃-10ₐ-_{o′}A_{do} | GalNAc₃- | A_{d} | 841 |
| | TₑₛGₑₒGₑₒTₑₒAₑₒA_{ds} | 10ₐ | | |
| | T_{ds}^{m}C_{ds}^{m}C_{ds}A_{ds}^{m}C_{ds} | | | |
| | T_{ds}T_{ds}T_{ds}^{m}C_{ds}Aₑₒ | | | |
| | GₑₒAₑₛGₑₛGₑ | | | |
| 678349 | GalNAc₃-13ₐ-_{o′}A_{do} | GalNAc₃- | A_{d} | 841 |
| | TₑₛGₑₒGₑₒTₑₒAₑₒA_{ds} | 13ₐ | | |
| | T_{ds}^{m}C_{ds}^{m}C_{ds}A_{ds}^{m}C_{ds} | | | |
| | T_{ds}T_{ds}T_{ds}^{m}C_{ds}AₑₒGₑₒ | | | |
| | AₑₛGₑₛGₑ | | | |

The structure of GalNAc₃-1ₐ was shown previously in Example 9, GalNAc₃-3ₐ was shown in Example 39, GalNAc₃-7ₐ was shown in Example 48, GalNAc₃-10ₐ was shown in Example 46, and GalNAc3-13ₐ was shown in Example 62.

### Treatment

Six to eight week old mice were each injected subcutaneously once per week at a dosage shown below, for a total of three doses, with an oligonucleotide listed below or with PBS. Each treatment group consisted of 4 animals. The mice were sacrificed 72 hours following the final dose. Factor XI liver mRNA levels were measured using real-time PCR and normalized to cyclophilin according to standard protocols. Liver transaminases, BUN, and bilirubin were also measured. The results below are presented as the average percent for each treatment group, normalized to the PBS control.

As illustrated in Table 78, treatment with antisense oligonucleotides lowered Factor XI liver mRNA in a dose-dependent manner. The results show that the oligonucleotides comprising a GalNAc conjugate were more potent than the parent lacking a GalNAc conjugate (ISIS 404071). Furthermore, the oligonucleotides comprising a 5′-GalNAc conjugate (ISIS 663086, 678347, 678348, and 678349) were even more potent than the oligonucleotide comprising a 3′-GalNAc conjugate (ISIS 656173).

TABLE 78
Factor XI liver mRNA, liver transaminase, BUN, and bilirubin levels

| | | Factor XI | | | | | | |
|---|---|---|---|---|---|---|---|---|
| ISIS | Dosage | mRNA | ALT | AST | BUN | Bilirubin | GalNAc₃ | SEQ |
| No. | (mg/kg) | (% PBS) | (U/L) | (U/L) | (mg/dL) | (mg/dL) | Cluster | ID No. |
| PBS | n/a | 100 | 63 | 70 | 21 | 0.18 | n/a | n/a |
| 404071 | 3 | 65 | 41 | 58 | 21 | 0.15 | n/a | 832 |
| | 10 | 33 | 49 | 53 | 23 | 0.15 | | |
| | 30 | 17 | 43 | 57 | 22 | 0.14 | | |
| 656173 | 0.7 | 43 | 90 | 89 | 21 | 0.16 | GalNAc₃-1a | 833 |
| | 2 | 9 | 36 | 58 | 26 | 0.17 | | |
| | 6 | 3 | 50 | 63 | 25 | 0.15 | | |
| 663086 | 0.7 | 33 | 91 | 169 | 25 | 0.16 | GalNAc₃-3a | 841 |
| | 2 | 7 | 38 | 55 | 21 | 0.16 | | |
| | 6 | 1 | 34 | 40 | 23 | 0.14 | | |
| 678347 | 0.7 | 35 | 28 | 49 | 20 | 0.14 | GalNAc₃-7a | 841 |
| | 2 | 10 | 180 | 149 | 21 | 0.18 | | |
| | 6 | 1 | 44 | 76 | 19 | 0.15 | | |
| 678348 | 0.7 | 39 | 43 | 54 | 21 | 0.16 | GalNAc₃-10a | 841 |
| | 2 | 5 | 38 | 55 | 22 | 0.17 | | |
| | 6 | 2 | 25 | 38 | 20 | 0.14 | | |
| 678349 | 0.7 | 34 | 39 | 46 | 20 | 0.16 | GalNAc₃-13a | 841 |
| | 2 | 8 | 43 | 63 | 21 | 0.14 | | |
| | 6 | 2 | 28 | 41 | 20 | 0.14 | | |

Example 84: Duration of Action In Vivo of Oligonucleotides Targeting Factor XI Comprising a GalNAc₃ Conjugate

The oligonucleotides listed in Table 77 were tested in a single dose study for duration of action in mice.

### Treatment

Six to eight week old mice were each injected subcutaneously once with an oligonucleotide listed in Table 77 or with PBS. Each treatment group consisted of 4 animals. Blood was drawn by tail bleeds the day before dosing to determine baseline and at 3, 10, and 17 days following the dose. Plasma Factor XI protein levels were measured by ELISA using Factor XI capture and biotinylated detection antibodies from R & D Systems, Minneapolis, MN (catalog #AF2460 and #BAF2460, respectively) and the OptEIA Reagent Set B (Catalog #550534, BD Biosciences, San Jose, CA). The results below are presented as the average percent of plasma Factor XI protein levels for each treatment group, normalized to baseline levels. The results show that the oligonucleotides comprising a GalNAc conjugate were more potent with longer duration of action than the parent lacking a GalNAc conjugate (ISIS 404071). Furthermore, the oligonucleotides comprising a 5′-GalNAc conjugate (ISIS 663086, 678347, 678348, and 678349) were even more potent with an even longer duration of action than the oligonucleotide comprising a 3′-GalNAc conjugate (ISIS 656173).

TABLE 79
Plasma Factor XI protein levels in mice

| | | Time point | | | | |
|---|---|---|---|---|---|---|
| ISIS | Dosage | (days post- | Factor XI | GalNAc₃ | | SEQ |
| No. | (mg/kg) | dose) | (% baseline) | Cluster | CM | ID No. |
| PBS | n/a | 3 | 123 | n/a | n/a | n/a |
| | | 10 | 56 | | | |
| | | 17 | 100 | | | |
| 404071 | 30 | 3 | 11 | n/a | n/a | 832 |
| | | 10 | 47 | | | |
| | | 17 | 52 | | | |
| 656173 | 6 | 3 | 1 | GalNAc₃-1a | A_{d} | 833 |
| | | 10 | 3 | | | |
| | | 17 | 21 | | | |
| 663086 | 6 | 3 | 1 | GalNAc₃-3a | A_{d} | 841 |
| | | 10 | 2 | | | |
| | | 17 | 9 | | | |
| 678347 | 6 | 3 | 1 | GalNAc₃-7a | A_{d} | 841 |
| | | 10 | 1 | | | |
| | | 17 | 8 | | | |
| 678348 | 6 | 3 | 1 | GalNAc₃-10a | A_{d} | 841 |
| | | 10 | 1 | | | |
| | | 17 | 6 | | | |
| 678349 | 6 | 3 | 1 | GalNAc₃-13a | A_{d} | 841 |
| | | 10 | 1 | | | |
| | | 17 | 5 | | | |

### Example 85: Antisense Inhibition In Vivo by Oligonucleotides Targeting SRB-1 Comprising a GalNAc₃ Conjugate

Oligonucleotides listed in Table 76 were tested in a dose-dependent study for antisense inhibition of SRB-1 in mice.

### Treatment

Six to eight week old C57BL/6 mice were each injected subcutaneously once per week at a dosage shown below, for a total of three doses, with an oligonucleotide listed in Table 76 or with saline. Each treatment group consisted of 4 animals. The mice were sacrificed 48 hours following the final administration to determine the SRB-1 mRNA levels using real-time PCR and RIBOGREEN® RNA quantification reagent (Molecular Probes, Inc. Eugene, OR) according to standard protocols. The results below are presented as the average percent of liver SRB-1 mRNA levels for each treatment group, normalized to the saline control.

As illustrated in Tables 80 and 81, treatment with antisense oligonucleotides lowered SRB-1 mRNA levels in a dose-dependent manner.

TABLE 80
SRB-1 mRNA in liver

| ISIS | Dosage | SRB-1 mRNA | GalNAc₃ | |
|---|---|---|---|---|
| No. | (mg/kg) | (% Saline) | Cluster | CM |
| Saline | n/a | 100 | n/a | n/a |
| 655861 | 0.1 | 94 | GalNAc₃-1a | A_{d} |
| | 0.3 | 119 | | |
| | 1 | 68 | | |
| | 3 | 32 | | |
| 661161 | 0.1 | 120 | GalNAc₃-3a | A_{d} |
| | 0.3 | 107 | | |
| | 1 | 68 | | |
| | 3 | 26 | | |
| 666881 | 0.1 | 107 | GalNAc₃-10a | A_{d} |
| | 0.3 | 107 | | |
| | 1 | 69 | | |
| | 3 | 27 | | |
| 666981 | 0.1 | 120 | GalNAc₃-7a | A_{d} |
| | 0.3 | 103 | | |
| | 1 | 54 | | |
| | 3 | 21 | | |
| 670061 | 0.1 | 118 | GalNAc₃-13a | A_{d} |
| | 0.3 | 89 | | |
| | 1 | 52 | | |
| | 3 | 18 | | |
| 677842 | 0.1 | 119 | GalNAc₃-20a | A_{d} |
| | 0.3 | 96 | | |
| | 1 | 65 | | |
| | 3 | 23 | | |

TABLE 81
SRB-1 mRNA in liver

| ISIS | Dosage | SRB-1 mRNA | GalNAc₃ | |
|---|---|---|---|---|
| No. | (mg/kg) | (% Saline) | Cluster | CM |
| 661161 | 0.1 | 107 | GalNAc₃-3a | A_{d} |
| | 0.3 | 95 | | |
| | 1 | 53 | | |
| | 3 | 18 | | |
| 677841 | 0.1 | 110 | GalNAc₃-19a | A_{d} |
| | 0.3 | 88 | | |
| | 1 | 52 | | |
| | 3 | 25 | | |

Liver transaminase levels, total bilirubin, BUN, and body weights were also measured using standard protocols. Average values for each treatment group are shown in Table 82 below.

TABLE 82

| ISIS | Dosage | ALT | AST | Bilirubin | BUN | Body Weight | GalNAc₃ | |
|---|---|---|---|---|---|---|---|---|
| No. | (mg/kg) | (U/L) | (U/L) | (mg/dL) | (mg/dL) | (% baseline) | Cluster | CM |
| Saline | n/a | 19 | 39 | 0.17 | 26 | 118 | n/a | n/a |
| 655861 | 0.1 | 25 | 47 | 0.17 | 27 | 114 | GalNAc₃-1a | A_{d} |
| | 0.3 | 29 | 56 | 0.15 | 27 | 118 | | |
| | 1 | 20 | 32 | 0.14 | 24 | 112 | | |
| | 3 | 27 | 54 | 0.14 | 24 | 115 | | |
| 661161 | 0.1 | 35 | 83 | 0.13 | 24 | 113 | GalNAc₃-3a | A_{d} |
| | 0.3 | 42 | 61 | 0.15 | 23 | 117 | | |
| | 1 | 34 | 60 | 0.18 | 22 | 116 | | |
| | 3 | 29 | 52 | 0.13 | 25 | 117 | | |
| 666881 | 0.1 | 30 | 51 | 0.15 | 23 | 118 | GalNAc₃-10a | A_{d} |
| | 0.3 | 49 | 82 | 0.16 | 25 | 119 | | |
| | 1 | 23 | 45 | 0.14 | 24 | 117 | | |
| | 3 | 20 | 38 | 0.15 | 21 | 112 | | |
| 666981 | 0.1 | 21 | 41 | 0.14 | 22 | 113 | GalNAc₃-7a | A_{d} |
| | 0.3 | 29 | 49 | 0.16 | 24 | 112 | | |
| | 1 | 19 | 34 | 0.15 | 22 | 111 | | |
| | 3 | 77 | 78 | 0.18 | 25 | 115 | | |
| 670061 | 0.1 | 20 | 63 | 0.18 | 24 | 111 | GalNAc₃-13a | A_{d} |
| | 0.3 | 20 | 57 | 0.15 | 21 | 115 | | |
| | 1 | 20 | 35 | 0.14 | 20 | 115 | | |
| | 3 | 27 | 42 | 0.12 | 20 | 116 | | |
| 677842 | 0.1 | 20 | 38 | 0.17 | 24 | 114 | GalNAc₃-20a | A_{d} |
| | 0.3 | 31 | 46 | 0.17 | 21 | 117 | | |
| | 1 | 22 | 34 | 0.15 | 21 | 119 | | |
| | 3 | 41 | 57 | 0.14 | 23 | 118 | | |

### Example 86: Antisense Inhibition In Vivo by Oligonucleotides Targeting TTR Comprising a GalNAc₃ Cluster

Oligonucleotides listed in Table 83 below were tested in a dose-dependent study for antisense inhibition of human transthyretin (TTR) in transgenic mice that express the human TTR gene.

### Treatment

Eight week old TTR transgenic mice were each injected subcutaneously once per week for three weeks, for a total of three doses, with an oligonucleotide and dosage listed in the tables below or with PBS. Each treatment group consisted of 4 animals. The mice were sacrificed 72 hours following the final administration. Tail bleeds were performed at various time points throughout the experiment, and plasma TTR protein, ALT, and AST levels were measured and reported in Tables 85-87. After the animals were sacrificed, plasma ALT, AST, and human TTR levels were measured, as were body weights, organ weights, and liver human TTR mRNA levels. TTR protein levels were measured using a clinical analyzer (AU480, Beckman Coulter, CA). Real-time PCR and RIBOGREEN® RNA quantification reagent (Molecular Probes, Inc. Eugene, OR) were used according to standard protocols to determine liver human TTR mRNA levels. The results presented in Tables 84-87 are the average values for each treatment group. The mRNA levels are the average values relative to the average for the PBS group. Plasma protein levels are the average values relative to the average value for the PBS group at baseline. Body weights are the average percent weight change from baseline until sacrifice for each individual treatment group. Organ weights shown are normalized to the animal's body weight, and the average normalized organ weight for each treatment group is then presented relative to the average normalized organ weight for the PBS group.

In Tables 84-87, “BL” indicates baseline, measurements that were taken just prior to the first dose. As illustrated in Tables 84 and 85, treatment with antisense oligonucleotides lowered TTR expression levels in a dose-dependent manner. The oligonucleotides comprising a GalNAc conjugate were more potent than the parent lacking a GalNAc conjugate (ISIS 420915). Furthermore, the oligonucleotides comprising a GalNAc conjugate and mixed PS/PG internucleoside linkages were even more potent than the oligonucleotide comprising a GalNAc conjugate and full PS linkages.

TABLE 83
Oligonucleotides targeting human TTR

| Isis | | | GalNAc | | SEQ |
|---|---|---|---|---|---|
| No. | Sequence 5′ to 3′ | Linkages | cluster | CM | ID No. |
| 420915 | Tₑₛ^{m}CₑₛTₑₛTₑₛGₑₛG_{ds}T_{ds}T_{ds}A_{ds}^{m}C_{ds} | PS | n/a | n/a | 842 |
| | A_{ds}T_{ds}G_{ds}A_{ds}A_{ds}AₑₛTₑₛ^{m}Cₑₛ^{m}Cₑₛ^{m}Cₑ | | | | |
| 660261 | Tₑₛ^{m}CₑₛTₑₛTₑₛGₑₛG_{ds}T_{ds}T_{ds}A_{ds} | PS | GalNAc₃-1a | A_{d} | 843 |
| | ^{m}C_{ds}A_{ds}T_{ds}G_{ds}A_{ds}A_{ds}AₑₛTₑₛ^{m}Cₑₛ | | | | |
| | ^{m}Cₑₛ^{m}CₑₒA_{do′}-GalNAc₃-1ₐ | | | | |
| 682883 | GalNAc₃-3_{a-o′}Tₑₛ^{m}CₑₒTₑₒ | PS/PO | GalNAc₃-3a | PO | 842 |
| | TₑₒGₑₒG_{ds}T_{ds}T_{ds}A_{ds}^{m}C_{ds}A_{ds}T_{ds} | | | | |
| | G_{ds}A_{ds}A_{ds}AₑₒTₑₒ^{m}Cₑₛ^{m}Cₑₛ^{m}Cₑ | | | | |
| 682884 | GalNAc₃-7_{a-o′}Tₑₛ^{m}CₑₒTₑₒ | PS/PO | GalNAc₃-7a | PO | 842 |
| | TₑₒGₑₒG_{ds}T_{ds}T_{ds}A_{ds}^{m}C_{ds}A_{ds}T_{ds} | | | | |
| | G_{ds}A_{ds}A_{ds}AₑₒTₑₒ^{m}Cₑₛ^{m}Cₑₛ^{m}Cₑ | | | | |
| 682885 | GalNAc₃-10_{a-o′}Tₑₛ^{m}CₑₒTₑₒ | PS/PO | GalNAc₃-10a | PO | 842 |
| | TₑₒGₑₒG_{ds}T_{ds}T_{ds}A_{ds}^{m}C_{ds}A_{ds}T_{ds} | | | | |
| | G_{ds}A_{ds}A_{ds}AₑₒTₑₒ^{m}Cₑₛ^{m}Cₑₛ^{m}Cₑ | | | | |
| 682886 | GalNAc₃-13_{a-o′}Tₑₛ^{m}CₑₒTₑₒ | PS/PO | GalNAc₃-13a | PO | 842 |
| | TₑₒGₑₒG_{ds}T_{ds}T_{ds}A_{ds}^{m}C_{ds}A_{ds}T_{ds} | | | | |
| | G_{ds}A_{ds}A_{ds}AₑₒTₑₒ^{m}Cₑₛ^{m}Cₑₛ^{m}Cₑ | | | | |
| 684057 | Tₑₛ^{m}CₑₒTₑₒTₑₒGₑₒG_{ds}T_{ds}T_{ds}A_{ds} | PS/PO | GalNAc₃-19a | A_{d} | 843 |
| | ^{m}C_{ds}A_{ds}T_{ds}G_{ds}A_{ds}A_{ds}AₑₒTₑₒ^{m}Cₑₛ | | | | |
| | ^{m}Cₑₛ^{m}CₑₒA_{do′}-GalNAc₃-19ₐ | | | | |

The legend for Table 85 can be found in Example 74. The structure of GalNAc₃-1 was shown in Example 9. The structure of GalNAC₃-3ₐ was shown in Example 39. The structure of GalNAc₃-7ₐ was shown in Example 48. The structure of GalNAc₃-10ₐ was shown in Example 46. The structure of GalNAc₃-13ₐ was shown in Example 62. The structure of GalNAc₃-19ₐ was shown in Example 70.

TABLE 84
Antisense inhibition of human TTR in vivo

| | | | Plasma TTR | | | |
|---|---|---|---|---|---|---|
| Isis | Dosage | TTR mRNA | protein | GalNAc | | SEQ |
| No. | (mg/kg) | (% PBS) | (% PBS) | cluster | CM | ID No. |
| PBS | n/a | 100 | 100 | n/a | n/a | |
| 420915 | 6 | 99 | 95 | n/a | n/a | 842 |
| | 20 | 48 | 65 | | | |
| | 60 | 18 | 28 | | | |
| 660261 | 0.6 | 113 | 87 | GalNAc₃-1a | A_{d} | 843 |
| | 2 | 40 | 56 | | | |
| | 6 | 20 | 27 | | | |
| | 20 | 9 | 11 | | | |

TABLE 85
Antisense inhibition of human TTR in vivo

| | | TTR | Plasma TTR protein (% PBS at BL) | | | | | | |
|---|---|---|---|---|---|---|---|---|---|
| Isis | Dosage | mRNA | | | | Day 17 | GalNAc | | SEQ |
| No. | (mg/kg) | (% PBS) | BL | Day 3 | Day 10 | (After sac) | cluster | CM | ID No. |
| PBS | n/a | 100 | 100 | 96 | 90 | 114 | n/a | n/a | |
| 420915 | 6 | 74 | 106 | 86 | 76 | 83 | n/a | n/a | 842 |
| | 20 | 43 | 102 | 66 | 61 | 58 | | | |
| | 60 | 24 | 92 | 43 | 29 | 32 | | | |
| 682883 | 0.6 | 60 | 88 | 73 | 63 | 68 | GalNAc₃-3a | PO | 842 |
| | 2 | 18 | 75 | 38 | 23 | 23 | | | |
| | 6 | 10 | 80 | 35 | 11 | 9 | | | |
| 682884 | 0.6 | 56 | 88 | 78 | 63 | 67 | GalNAc₃-7a | PO | 842 |
| | 2 | 19 | 76 | 44 | 25 | 23 | | | |
| | 6 | 15 | 82 | 35 | 21 | 24 | | | |
| 682885 | 0.6 | 60 | 92 | 77 | 68 | 76 | GalNAc₃-10a | PO | 842 |
| | 2 | 22 | 93 | 58 | 32 | 32 | | | |
| | 6 | 17 | 85 | 37 | 25 | 20 | | | |
| 682886 | 0.6 | 57 | 91 | 70 | 64 | 69 | GalNAc₃-13a | PO | 842 |
| | 2 | 21 | 89 | 50 | 31 | 30 | | | |
| | 6 | 18 | 102 | 41 | 24 | 27 | | | |
| 684057 | 0.6 | 53 | 80 | 69 | 56 | 62 | GalNAc₃-19a | A_{d} | 843 |
| | 2 | 21 | 92 | 55 | 34 | 30 | | | |
| | 6 | 11 | 82 | 50 | 18 | 13 | | | |

TABLE 86
Transaminase levels, body weight changes, and relative organ weights

| | | ALT (U/L) | | | | AST (U/L) | | | | | | | | |
|---|---|---|---|---|---|---|---|---|---|---|---|---|---|---|
| Isis | Dosage | | Day | Day | Day | | Day | Day | Day | Body | Liver | Spleen | Kidney | SEQ |
| No. | (mg/kg) | BL | 3 | 10 | 17 | BL | 3 | 10 | 17 | (% BL) | (% PBS) | (% PBS) | (% PBS) | ID No. |
| PBS | n/a | 33 | 34 | 33 | 24 | 58 | 62 | 67 | 52 | 105 | 100 | 100 | 100 | n/a |
| 420915 | 6 | 34 | 33 | 27 | 21 | 64 | 59 | 73 | 47 | 115 | 99 | 89 | 91 | 842 |
| | 20 | 34 | 30 | 28 | 19 | 64 | 54 | 56 | 42 | 111 | 97 | 83 | 89 | |
| | 60 | 34 | 35 | 31 | 24 | 61 | 58 | 71 | 58 | 113 | 102 | 98 | 95 | |
| 660261 | 0.6 | 33 | 38 | 28 | 26 | 70 | 71 | 63 | 59 | 111 | 96 | 99 | 92 | 843 |
| | 2 | 29 | 32 | 31 | 34 | 61 | 60 | 68 | 61 | 118 | 100 | 92 | 90 | |
| | 6 | 29 | 29 | 28 | 34 | 58 | 59 | 70 | 90 | 114 | 99 | 97 | 95 | |
| | 20 | 33 | 32 | 28 | 33 | 64 | 54 | 68 | 95 | 114 | 101 | 106 | 92 | |

TABLE 87
Transaminase levels, body weight changes, and relative organ weights

| | | ALT (U/L) | | | | AST (U/L) | | | | | | | | |
|---|---|---|---|---|---|---|---|---|---|---|---|---|---|---|
| Isis | Dosage | | Day | Day | Day | | Day | Day | Day | Body | Liver | Spleen | Kidney | SEQ |
| No. | (mg/kg) | BL | 3 | 10 | 17 | BL | 3 | 10 | 17 | (% BL) | (% PBS) | (% PBS) | (% PBS) | ID No. |
| PBS | n/a | 32 | 34 | 37 | 41 | 62 | 78 | 76 | 77 | 104 | 100 | 100 | 100 | n/a |
| 420915 | 6 | 32 | 30 | 34 | 34 | 61 | 71 | 72 | 66 | 102 | 103 | 102 | 105 | 842 |
| | 20 | 41 | 34 | 37 | 33 | 80 | 76 | 63 | 54 | 106 | 107 | 135 | 101 | |
| | 60 | 36 | 30 | 32 | 34 | 58 | 81 | 57 | 60 | 106 | 105 | 104 | 99 | |
| 682883 | 0.6 | 32 | 35 | 38 | 40 | 53 | 81 | 74 | 76 | 104 | 101 | 112 | 95 | 842 |
| | 2 | 38 | 39 | 42 | 43 | 71 | 84 | 70 | 77 | 107 | 98 | 116 | 99 | |
| | 6 | 35 | 35 | 41 | 38 | 62 | 79 | 103 | 65 | 105 | 103 | 143 | 97 | |
| 682884 | 0.6 | 33 | 32 | 35 | 34 | 70 | 74 | 75 | 67 | 101 | 100 | 130 | 99 | 842 |
| | 2 | 31 | 32 | 38 | 38 | 63 | 77 | 66 | 55 | 104 | 103 | 122 | 100 | |
| | 6 | 38 | 32 | 36 | 34 | 65 | 85 | 80 | 62 | 99 | 105 | 129 | 95 | |
| 682885 | 0.6 | 39 | 26 | 37 | 35 | 63 | 63 | 77 | 59 | 100 | 109 | 109 | 112 | 842 |
| | 2 | 30 | 26 | 38 | 40 | 54 | 56 | 71 | 72 | 102 | 98 | 111 | 102 | |
| | 6 | 27 | 27 | 34 | 35 | 46 | 52 | 56 | 64 | 102 | 98 | 113 | 96 | |
| 682886 | 0.6 | 30 | 40 | 34 | 36 | 58 | 87 | 54 | 61 | 104 | 99 | 120 | 101 | 842 |
| | 2 | 27 | 26 | 34 | 36 | 51 | 55 | 55 | 69 | 103 | 91 | 105 | 92 | |
| | 6 | 40 | 28 | 34 | 37 | 107 | 54 | 61 | 69 | 109 | 100 | 102 | 99 | |
| 684057 | 0.6 | 35 | 26 | 33 | 39 | 56 | 51 | 51 | 69 | 104 | 99 | 110 | 102 | 843 |
| | 2 | 33 | 32 | 31 | 40 | 54 | 57 | 56 | 87 | 103 | 100 | 112 | 97 | |
| | 6 | 39 | 33 | 35 | 40 | 67 | 52 | 55 | 92 | 98 | 104 | 121 | 108 | |

### Example 87: Duration of Action In Vivo by Single Doses of Oligonucleotides Targeting TTR Comprising a GalNAc₃ Cluster

ISIS numbers 420915 and 660261 (see Table 83) were tested in a single dose study for duration of action in mice. ISIS numbers 420915, 682883, and 682885 (see Table 83) were also tested in a single dose study for duration of action in mice.

### Treatment

Eight week old, male transgenic mice that express human TTR were each injected subcutaneously once with 100 mg/kg ISIS No. 420915 or 13.5 mg/kg ISIS No. 660261. Each treatment group consisted of 4 animals. Tail bleeds were performed before dosing to determine baseline and at days 3, 7, 10, 17, 24, and 39 following the dose. Plasma TTR protein levels were measured as described in Example 86. The results below are presented as the average percent of plasma TTR levels for each treatment group, normalized to baseline levels.

TABLE 88
Plasma TTR protein levels

| | | Time point | | | | |
|---|---|---|---|---|---|---|
| ISIS | Dosage | (days post- | TTR | GalNAc₃ | | SEQ |
| No. | (mg/kg) | dose) | (% baseline) | Cluster | CM | ID No. |
| 420915 | 100 | 3 | 30 | n/a | n/a | 842 |
| | | 7 | 23 | | | |
| | | 10 | 35 | | | |
| | | 17 | 53 | | | |
| | | 24 | 75 | | | |
| | | 39 | 100 | | | |
| 660261 | 13.5 | 3 | 27 | GalNAc₃-1a | A_{d} | 843 |
| | | 7 | 21 | | | |
| | | 10 | 22 | | | |
| | | 17 | 36 | | | |
| | | 24 | 48 | | | |
| | | 39 | 69 | | | |

### Treatment

Female transgenic mice that express human TTR were each injected subcutaneously once with 100 mg/kg ISIS No. 420915, 10.0 mg/kg ISIS No. 682883, or 10.0 mg/kg 682885. Each treatment group consisted of 4 animals. Tail bleeds were performed before dosing to determine baseline and at days 3, 7, 10, 17, 24, and 39 following the dose. Plasma TTR protein levels were measured as described in Example 86. The results below are presented as the average percent of plasma TTR levels for each treatment group, normalized to baseline levels.

TABLE 89
Plasma TTR protein levels

| | | Time point | | | | |
|---|---|---|---|---|---|---|
| ISIS | Dosage | (days post- | TTR | GalNAc₃ | | SEQ |
| No. | (mg/kg) | dose) | (% baseline) | Cluster | CM | ID No. |
| 420915 | 100 | 3 | 48 | n/a | n/a | 842 |
| | | 7 | 48 | | | |
| | | 10 | 48 | | | |
| | | 17 | 66 | | | |
| | | 31 | 80 | | | |
| 682883 | 10.0 | 3 | 45 | GalNAc₃-3a | PO | 842 |
| | | 7 | 37 | | | |
| | | 10 | 38 | | | |
| | | 17 | 42 | | | |
| | | 31 | 65 | | | |
| 682885 | 10.0 | 3 | 40 | GalNAc₃-10a | PO | 842 |
| | | 7 | 33 | | | |
| | | 10 | 34 | | | |
| | | 17 | 40 | | | |
| | | 31 | 64 | | | |

The results in Tables 88 and 89 show that the oligonucleotides comprising a GalNAc conjugate are more potent with a longer duration of action than the parent oligonucleotide lacking a conjugate (ISIS 420915).

### Example 88: Splicing Modulation In Vivo by Oligonucleotides Targeting SMN Comprising a GalNAc₃ Conjugate

The oligonucleotides listed in Table 90 were tested for splicing modulation of human survival of motor neuron (SMN) in mice.

TABLE 90
Modified ASOs targeting SMN

| ISIS | Sequences | GalNAc₃ | | SEQ |
|---|---|---|---|---|
| No. | (5′ to 3′) | Cluster | CM | ID No. |
| 387954 | AₑₛTₑₛTₑₛ^{m}CₑₛAₑₛ | n/a | n/a | 844 |
| | ^{m}CₑₛTₑₛTₑₛTₑₛ^{m}Cₑₛ | | | |
| | AₑₛTₑₛAₑₛAₑₛTₑₛ | | | |
| | Gₑₛ^{m}CₑₛTₑₛGₑₛGₑ | | | |
| 699819 | GalNAc₃-7ₐ-_{o′}Aₑₛ | GalNAc₃- | PO | 844 |
| | TₑₛTₑₛ^{m}CₑₛAₑₛ^{m}CₑₛTₑₛ | 7a | | |
| | TₑₛTₑₛ^{m}CₑₛAₑₛTₑₛAₑₛ | | | |
| | AₑₛTₑₛGₑₛ^{m}CₑₛTₑₛGₑₛGₑ | | | |
| 699821 | GalNAc₃-7ₐ-_{o′}AₑₛTₑₒ | GalNAc₃- | PO | 844 |
| | Tₑₒ^{m}CₑₒAₑₒ^{m}CₑₒTₑₒTₑₒ | 7a | | |
| | Tₑₒ^{m}CₑₒAₑₒTₑₒAₑₒAₑₒ | | | |
| | TₑₒGₑₒ^{m}CₑₒTₑₛGₑₛGₑ | | | |
| 700000 | AₑₛTₑₛTₑₛ^{m}CₑₛAₑₛ^{m}Cₑₛ | GalNAc₃- | A_{d} | 845 |
| | TₑₛTₑₛTₑₛ^{m}CₑₛAₑₛTₑₛAₑₛ | 1a | | |
| | AₑₛTₑₛGₑₛ^{m}CₑₛTₑₛGₑₛ | | | |
| | GₑₒA_{do′}-GalNAc₃-1ₐ | | | |
| 703421 | X-ATT^{m}CA^{m}CTTT^{m}C | n/a | n/a | 844 |
| | ATAATG^{m}CTGG | | | |
| 703422 | GalNAc₃-7_{b}-X- | GalNAc₃- | n/a | 844 |
| | ATT^{m}CA^{m}CTTT^{m}C | 7b | | |
| | ATAATG^{m}CTGG | | | |

The structure of GalNAc₃-7ₐ was shown previously in Example 48. “X” indicates a primary amine generated by Gene Tools (Philomath, OR), and GalNAc₃-7b indicates the structure of GalNAc₃-7ₐ lacking the —NH—C₆—O portion of the linker as shown below:

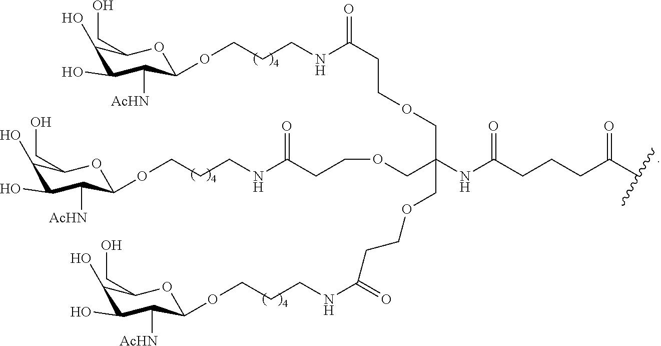

ISIS numbers 703421 and 703422 are morphlino oligonucleotides, wherein each nucleotide of the two oligonucleotides is a morpholino nucleotide.

### Treatment

Six week old transgenic mice that express human SMN were injected subcutaneously once with an oligonucleotide listed in Table 91 or with saline. Each treatment group consisted of 2 males and 2 females. The mice were sacrificed 3 days following the dose to determine the liver human SMN mRNA levels both with and without exon 7 using real-time PCR according to standard protocols. Total RNA was measured using Ribogreen reagent. The SMN mRNA levels were normalized to total mRNA, and further normalized to the averages for the saline treatment group. The resulting average ratios of SMN mRNA including exon 7 to SMN mRNA missing exon 7 are shown in Table 91. The results show that fully modified oligonucleotides that modulate splicing and comprise a GalNAc conjugate are significantly more potent in altering splicing in the liver than the parent oligonucleotides lacking a GlaNAc conjugate. Furthermore, this trend is maintained for multiple modification chemistries, including 2′-MOE and morpholino modified oligonucleotides.

TABLE 91
Effect of oligonucleotides targeting human SMN in vivo

| ISIS | Dose | | GalNAc₃ | | SEQ |
|---|---|---|---|---|---|
| No. | (mg/kg) | +Exon 7/−Exon 7 | Cluster | CM | ID No. |
| Saline | n/a | 1.00 | n/a | n/a | n/a |
| 387954 | 32 | 1.65 | n/a | n/a | 844 |
| 387954 | 288 | 5.00 | n/a | n/a | 844 |
| 699819 | 32 | 7.84 | GalNAc₃-7a | PO | 844 |
| 699821 | 32 | 7.22 | GalNAc₃-7a | PO | 844 |
| 700000 | 32 | 6.91 | GalNAc₃-1a | A_{d} | 845 |
| 703421 | 32 | 1.27 | n/a | n/a | 844 |
| 703422 | 32 | 4.12 | GalNAc₃-7b | n/a | 844 |

### Example 89: Antisense Inhibition In Vivo by Oligonucleotides Targeting Apolipoprotein a (Apo(a)) Comprising a GalNAc₃ Conjugate

The oligonucleotides listed in Table 92 below were tested in a study for dose-dependent inhibition of Apo(a) in transgenic mice.

TABLE 92
Modified ASOs targeting Apo(a)

| ISIS | Sequences | GalNAc₃ | | SEQ |
|---|---|---|---|---|
| No. | (5′ to 3′) | Cluster | CM | ID No. |
| 494372 | TₑₛGₑₛ^{m}CₑₛTₑₛ^{m}Cₑₛ | n/a | n/a | 847 |
| | ^{m}C_{ds}G_{ds}T_{ds}T_{ds}G_{ds} | | | |
| | G_{ds}T_{ds}G_{ds}^{m}C_{ds}T_{ds} | | | |
| | TₑₛGₑₛTₑₛTₑₛ^{m}Cₑ | | | |
| 681257 | GalNAc₃-7ₐ-_{o′}Tₑₛ | GalNAc₃- | PO | 847 |
| | Gₑₒ^{m}CₑₒTₑₒ^{m}Cₑₒ^{m}C_{ds}G_{ds} | 7a | | |
| | T_{ds}T_{ds}G_{ds}G_{ds}T_{ds}G_{ds}^{m}C_{ds} | | | |
| | T_{ds}TₑₒGₑₒTₑₛTₑₛ^{m}Cₑ | | | |

The structure of GalNAc₃-7ₐ was shown in Example 48.

### Treatment

Eight week old, female C57BL/6 mice (Jackson Laboratory, Bar Harbor, ME) were each injected subcutaneously once per week at a dosage shown below, for a total of six doses, with an oligonucleotide listed in Table 92 or with PBS. Each treatment group consisted of 3-4 animals. Tail bleeds were performed the day before the first dose and weekly following each dose to determine plasma Apo(a) protein levels. The mice were sacrificed two days following the final administration. Apo(a) liver mRNA levels were determined using real-time PCR and RIBOGREEN® RNA quantification reagent (Molecular Probes, Inc. Eugene, OR) according to standard protocols. Apo(a) plasma protein levels were determined using ELISA, and liver transaminase levels were determined. The mRNA and plasma protein results in Table 93 are presented as the treatment group average percent relative to the PBS treated group. Plasma protein levels were further normalized to the baseline (BL) value for the PBS group. Average absolute transaminase levels and body weights (% relative to baseline averages) are reported in Table 94.

As illustrated in Table 93, treatment with the oligonucleotides lowered Apo(a) liver mRNA and plasma protein levels in a dose-dependent manner. Furthermore, the oligonucleotide comprising the GalNAc conjugate was significantly more potent with a longer duration of action than the parent oligonucleotide lacking a GalNAc conjugate. As illustrated in Table 94, transaminase levels and body weights were unaffected by the oligonucleotides, indicating that the oligonucleotides were well tolerated.

TABLE 93
Apo(a) liver mRNA and plasma protein levels

| | | Apo(a) | Apo(a) plasma protein (% PBS) | | | | | | |
|---|---|---|---|---|---|---|---|---|---|
| ISIS | Dosage | mRNA | | Week | Week | Week | Week | Week | Week |
| No. | (mg/kg) | (% PBS) | BL | 1 | 2 | 3 | 4 | 5 | 6 |
| PBS | n/a | 100 | 100 | 120 | 119 | 113 | 88 | 121 | 97 |
| 494372 | 3 | 80 | 84 | 89 | 91 | 98 | 87 | 87 | 79 |
| | 10 | 30 | 87 | 72 | 76 | 71 | 57 | 59 | 46 |
| | 30 | 5 | 92 | 54 | 28 | 10 | 7 | 9 | 7 |
| 681257 | 0.3 | 75 | 79 | 76 | 89 | 98 | 71 | 94 | 78 |
| | 1 | 19 | 79 | 88 | 66 | 60 | 54 | 32 | 24 |
| | 3 | 2 | 82 | 52 | 17 | 7 | 4 | 6 | 5 |
| | 10 | 2 | 79 | 17 | 6 | 3 | 2 | 4 | 5 |

TABLE 94

| ISIS | Dosage | ALT | AST | Body weight |
|---|---|---|---|---|
| No. | (mg/kg) | (U/L) | (U/L) | (% baseline) |
| PBS | n/a | 37 | 54 | 103 |
| 494372 | 3 | 28 | 68 | 106 |
| | 10 | 22 | 55 | 102 |
| | 30 | 19 | 48 | 103 |
| 681257 | 0.3 | 30 | 80 | 104 |
| | 1 | 26 | 47 | 105 |
| | 3 | 29 | 62 | 102 |
| | 10 | 21 | 52 | 107 |

### Example 90: Antisense Inhibition In Vivo by Oligonucleotides Targeting TTR Comprising a GalNAc₃ Cluster

Oligonucleotides listed in Table 95 below were tested in a dose-dependent study for antisense inhibition of human transthyretin (TTR) in transgenic mice that express the human TTR gene.

### Treatment

TTR transgenic mice were each injected subcutaneously once per week for three weeks, for a total of three doses, with an oligonucleotide and dosage listed in Table 96 or with PBS. Each treatment group consisted of 4 animals. Prior to the first dose, a tail bleed was performed to determine plasma TTR protein levels at baseline (BL). The mice were sacrificed 72 hours following the final administration. TTR protein levels were measured using a clinical analyzer (AU480, Beckman Coulter, CA). Real-time PCR and RIBOGREEN® RNA quantification reagent (Molecular Probes, Inc. Eugene, OR) were used according to standard protocols to determine liver human TTR mRNA levels. The results presented in Table 96 are the average values for each treatment group. The mRNA levels are the average values relative to the average for the PBS group. Plasma protein levels are the average values relative to the average value for the PBS group at baseline. “BL” indicates baseline, measurements that were taken just prior to the first dose. As illustrated in Table 96, treatment with antisense oligonucleotides lowered TTR expression levels in a dose-dependent manner. The oligonucleotides comprising a GalNAc conjugate were more potent than the parent lacking a GalNAc conjugate (ISIS 420915), and oligonucleotides comprising a phosphodiester or deoxyadenosine cleavable moiety showed significant improvements in potency compared to the parent lacking a conjugate (see ISIS numbers 682883 and 666943 vs 420915 and see Examples 86 and 87).

TABLE 95
Oligonucleotides targeting human TTR

| Isis | | | GalNAc | | SEQ |
|---|---|---|---|---|---|
| No. | Sequence 5′ to 3′ | Linkages | cluster | CM | ID No. |
| 420915 | Tₑₛ^{m}CₑₛTₑₛTₑₛGₑₛG_{ds}T_{ds}T_{ds}A_{ds}^{m}C_{ds} | PS | n/a | n/a | 842 |
| | A_{ds}T_{ds}G_{ds}A_{ds}A_{ds}AₑₛTₑₛ^{m}Cₑₛ^{m}Cₑₛ^{m}Cₑ | | | | |
| 682883 | GalNAc₃-3_{a-o′}Tₑₛ^{m}CₑₒTₑₒTₑₒ | PS/PO | GalNAc₃-3a | PO | 842 |
| | GₑₒG_{ds}T_{ds}T_{ds}A_{ds}^{m}C_{ds}A_{ds}T_{ds} | | | | |
| | G_{ds}A_{ds}A_{ds}AₑₒTₑₒ^{m}Cₑₛ^{m}Cₑₛ^{m}Cₑ | | | | |
| 666943 | GalNAc₃-3_{a-o′}A_{do}Tₑₛ^{m}CₑₒTₑₒ | PS/PO | GalNAc₃-3a | A_{d} | 846 |
| | TₑₒGₑₒG_{ds}T_{ds}T_{ds}A_{ds}^{m}C_{ds}A_{ds}T_{ds} | | | | |
| | G_{ds}A_{ds}A_{ds}AₑₒTₑₒ^{m}Cₑₛ^{m}Cₑₛ^{m}Cₑ | | | | |
| 682887 | GalNAc₃-7_{a-o′}A_{do}Tₑₛ^{m}CₑₒTₑₒ | PS/PO | GalNAc₃-7a | A_{d} | 846 |
| | TₑₒGₑₒG_{ds}T_{ds}T_{ds}A_{ds}^{m}C_{ds}A_{ds}T_{ds} | | | | |
| | G_{ds}A_{ds}A_{ds}AₑₒTₑₒ^{m}Cₑₛ^{m}Cₑₛ^{m}Cₑ | | | | |
| 682888 | GalNAc₃-10_{a-o′}A_{do}Tₑₛ^{m}Cₑₒ | PS/PO | GalNAc₃-10a | A_{d} | 846 |
| | TₑₒTₑₒGₑₒG_{ds}T_{ds}T_{ds}A_{ds}^{m}C_{ds}A_{ds} | | | | |
| | T_{ds}G_{ds}A_{ds}A_{ds}AₑₒTₑₒ^{m}Cₑₛ^{m}Cₑₛ^{m}Cₑ | | | | |
| 682889 | GalNAc₃-13_{a-o′}A_{do}Tₑₛ^{m}Cₑₒ | PS/PO | GalNAc₃-13a | A_{d} | 846 |
| | TₑₒTₑₒGₑₒG_{ds}T_{ds}T_{ds}A_{ds}^{m}C_{ds}A_{ds} | | | | |
| | T_{ds}G_{ds}A_{ds}A_{ds}AₑₒTₑₒ^{m}Cₑₛ^{m}Cₑₛ^{m}Cₑ | | | | |

The legend for Table 95 can be found in Example 74. The structure of GalNAc₃-3ₐ was shown in Example 39. The structure of GalNAc₃-7ₐ was shown in Example 48. The structure of GalNAc₃-10ₐ was shown in Example 46. The structure of GalNAc₃-13ₐ was shown in Example 62.

TABLE 96
Antisense inhibition of human TTR in vivo

| | | TTR | TTR | | |
|---|---|---|---|---|---|
| Isis | Dosage | mRNA | protein | GalNAc | |
| No. | (mg/kg) | (% PBS) | (% BL) | cluster | CM |
| PBS | n/a | 100 | 124 | n/a | n/a |
| 420915 | 6 | 69 | 114 | n/a | n/a |
| | 20 | 71 | 86 | | |
| | 60 | 21 | 36 | | |
| 682883 | 0.6 | 61 | 73 | GalNAc₃-3a | PO |
| | 2 | 23 | 36 | | |
| | 6 | 18 | 23 | | |
| 666943 | 0.6 | 74 | 93 | GalNAc₃-3a | A_{d} |
| | 2 | 33 | 57 | | |
| | 6 | 17 | 22 | | |
| 682887 | 0.6 | 60 | 97 | GalNAc₃-7a | A_{d} |
| | 2 | 36 | 49 | | |
| | 6 | 12 | 19 | | |
| 682888 | 0.6 | 65 | 92 | GalNAc₃-10a | A_{d} |
| | 2 | 32 | 46 | | |
| | 6 | 17 | 22 | | |
| 682889 | 0.6 | 72 | 74 | GalNAc₃-13a | A_{d} |
| | 2 | 38 | 45 | | |
| | 6 | 16 | 18 | | |

### Example 91: Antisense Inhibition In Vivo by Oligonucleotides Targeting Factor VII Comprising a GalNAc₃ Conjugate in Non-Human Primates

Oligonucleotides listed in Table 97 below were tested in a non-terminal, dose escalation study for antisense inhibition of Factor VII in monkeys.

### Treatment

Non-naive monkeys were each injected subcutaneously on days 0, 15, and 29 with escalating doses of an oligonucleotide listed in Table 97 or with PBS. Each treatment group consisted of 4 males and 1 female. Prior to the first dose and at various time points thereafter, blood draws were performed to determine plasma Factor VII protein levels. Factor VII protein levels were measured by ELISA. The results presented in Table 98 are the average values for each treatment group relative to the average value for the PBS group at baseline (BL), the measurements taken just prior to the first dose. As illustrated in Table 98, treatment with antisense oligonucleotides lowered Factor VII expression levels in a dose-dependent manner, and the oligonucleotide comprising the GalNAc conjugate was significantly more potent in monkeys compared to the oligonucleotide lacking a GalNAc conjugate.

TABLE 97
Oligonucleotides targeting Factor VII

| Isis | | | GalNAc | | SEQ |
|---|---|---|---|---|---|
| No. | Sequence 5′ to 3′ | Linkages | cluster | CM | ID No. |
| 407935 | AₑₛTₑₛGₑₛ^{m}CₑₛAₑₛT_{ds}G_{ds}G_{ds}T_{ds}G_{ds} | PS | n/a | n/a | 848 |
| | A_{ds}T_{ds}G_{ds}^{m}C_{ds}T_{ds}Tₑₛ^{m}CₑₛTₑₛGₑₛAₑ | | | | |
| 686892 | GalNAc₃-10_{a-o′} | PS | GalNAc₃-10a | PO | 848 |
| | AₑₛTₑₛGₑₛ^{m}CₑₛAₑₛT_{ds}G_{ds}G_{ds}T_{ds}G_{ds} | | | | |
| | A_{ds}T_{ds}G_{ds}^{m}C_{ds}T_{ds}Tₑₛ^{m}CₑₛTₑₛGₑₛAₑ | | | | |

The legend for Table 97 can be found in Example 74. The structure of GalNAc₃-10ₐ was shown in Example 46.

TABLE 98
Factor VII plasma protein levels

| ISIS | | Dose | Factor VII |
|---|---|---|---|
| No. | Day | (mg/kg) | (% BL) |
| 407935 | 0 | n/a | 100 |
| | 15 | 10 | 87 |
| | 22 | n/a | 92 |
| | 29 | 30 | 77 |
| | 36 | n/a | 46 |
| | 43 | n/a | 43 |
| 686892 | 0 | 3 | 100 |
| | 15 | 10 | 56 |
| | 22 | n/a | 29 |
| | 29 | 30 | 19 |
| | 36 | n/a | 15 |
| | 43 | n/a | 11 |

### Example 92: Antisense Inhibition in Primary Hepatocytes by Antisense Oligonucleotides Targeting Apo-CIII Comprising a GalNAc₃ Conjugate

Primary mouse hepatocytes were seeded in 96-well plates at 15,000 cells per well, and the oligonucleotides listed in Table 99, targeting mouse ApoC-II, were added at 0.46, 1.37, 4.12, or 12.35, 37.04, 111.11, or 333.33 nM or 1.00 μM. After incubation with the oligonucleotides for 24 hours, the cells were lysed and total RNA was purified using RNeasy (Qiagen). ApoC-II mRNA levels were determined using real-time PCR and RIBOGREEN® RNA quantification reagent (Molecular Probes, Inc.) according to standard protocols. IC₅₀ values were determined using Prism 4 software (GraphPad). The results show that regardless of whether the cleavable moiety was a phosphodiester or a phosphodiester-linked deoxyadensoine, the oligonucleotides comprising a GalNAc conjugate were significantly more potent than the parent oligonucleotide lacking a conjugate.

TABLE 99
Inhibition of mouse APOC-III
expression in mouse primary hepatocytes

| ISIS | Sequence | | IC₅₀ | SEQ |
|---|---|---|---|---|
| No. | (5′ to 3′) | CM | (nM) | ID No. |
| 440670 | ^{m}CₑₛAₑₛGₑₛ^{m}CₑₛTₑₛT_{ds}T_{ds} | n/a | 13.20 | 849 |
| | A_{ds}T_{ds}T_{ds}A_{ds}G_{ds}G_{ds}G_{ds} | | | |
| | A_{ds}^{m}CₑₛAₑₛGₑₛ^{m}CₑₛAₑ | | | |
| 661180 | ^{m}CₑₛAₑₛGₑₛ^{m}CₑₛTₑₛT_{ds}T_{ds} | A_{d} | 1.40 | 850 |
| | A_{ds}T_{ds}T_{ds}A_{ds}G_{ds}G_{ds}G_{ds} | | | |
| | A_{ds}^{m}CₑₛAₑₛGₑₛ^{m}CₑₛAₑₒ | | | |
| | A_{do′}-GalNAc₃-1ₐ | | | |
| 680771 | GalNAc₃-3_{a-o′}^{m}CₑₛAₑₛ | PO | 0.70 | 849 |
| | Gₑₛ^{m}CₑₛTₑₛT_{ds}T_{ds}A_{ds}T_{ds} | | | |
| | T_{ds}A_{ds}G_{ds}G_{ds}G_{ds}A_{ds}^{m}Cₑₛ | | | |
| | AₑₛGₑₛ^{m}CₑₛAₑ | | | |
| 680772 | GalNAc₃-7_{a-o′}^{m}CₑₛAₑₛ | PO | 1.70 | 849 |
| | Gₑₛ^{m}CₑₛTₑₛT_{ds}T_{ds}A_{ds}T_{ds} | | | |
| | T_{ds}A_{ds}G_{ds}G_{ds}G_{ds}A_{ds}^{m}Cₑₛ | | | |
| | AₑₛGₑₛ^{m}CₑₛAₑ | | | |
| 680773 | GalNAc₃-10_{a-o′}^{m}CₑₛAₑₛ | PO | 2.00 | 849 |
| | Gₑₛ^{m}CₑₛTₑₛT_{ds}T_{ds}A_{ds}T_{ds} | | | |
| | T_{ds}A_{ds}G_{ds}G_{ds}G_{ds}A_{ds}^{m}Cₑₛ | | | |
| | AₑₛGₑₛ^{m}CₑₛAₑ | | | |
| 680774 | GalNAc₃-13_{a-o′}^{m}CₑₛAₑₛ | PO | 1.50 | 849 |
| | Gₑₛ^{m}CₑₛTₑₛT_{ds}T_{ds}A_{ds}T_{ds} | | | |
| | T_{ds}A_{ds}G_{ds}G_{ds}G_{ds}A_{ds}^{m}Cₑₛ | | | |
| | AₑₛGₑₛ^{m}CₑₛAₑ | | | |
| 681272 | GalNAc₃-3_{a-o′}^{m}CₑₛAₑₒ | PO | <0.46 | 849 |
| | Gₑₒ^{m}CₑₒTₑₒT_{ds}T_{ds}A_{ds}T_{ds} | | | |
| | T_{ds}A_{ds}G_{ds}G_{ds}G_{ds}A_{ds}^{m}Cₑₒ | | | |
| | AₑₒGₑₛ^{m}CₑₛAₑ | | | |
| 681273 | GalNAc₃-3ₐ-_{o′}A_{do}^{m}Cₑₛ | A_{d} | 1.10 | 851 |
| | AₑₛGₑₛ^{m}CₑₛTₑₛT_{ds}T_{ds}A_{ds} | | | |
| | T_{ds}T_{ds}A_{ds}G_{ds}G_{ds}G_{ds}A_{ds} | | | |
| | ^{m}CₑₛAₑₛGₑₛ^{m}CₑₛAₑ | | | |
| 683733 | ^{m}CₑₛAₑₛGₑₛ^{m}CₑₛTₑₛT_{ds} | A_{d} | 2.50 | 850 |
| | T_{ds}A_{ds}T_{ds}T_{ds}A_{ds}G_{ds}G_{ds} | | | |
| | G_{ds}A_{ds}^{m}CₑₛAₑₛGₑₛ^{m}CₑₛAₑₒ | | | |
| | A_{do′}-GalNAc₃-19ₐ | | | |

The structure of GalNAc₃-1ₐ was shown previously in Example 9, GalNAc₃-3ₐ was shown in Example 39, GalNAc₃-7ₐ was shown in Example 48, GalNAc₃-10ₐ was shown in Example 46, GalNAc₃-13ₐ was shown in Example 62, and GalNAc₃-19ₐ was shown in Example 70.

### Example 93: Antisense Inhibition In Vivo by Oligonucleotides Targeting SRB-1 Comprising Mixed Wings and a 5′-GalNAc₃ Conjugate

The oligonucleotides listed in Table 100 were tested in a dose-dependent study for antisense inhibition of SRB-1 in mice.

TABLE 100
Modified ASOs targeting SRB-1

| ISIS | Sequences | GalNAc₃ | | SEQ |
|---|---|---|---|---|
| No. | (5′ to 3′) | Cluster | CM | ID No. |
| 449093 | TₖₛTₖₛ^{m}CₖₛA_{ds}G_{ds} | n/a | n/a | 852 |
| | T_{ds}^{m}C_{ds}A_{ds}T_{ds}G_{ds}A_{ds} | | | |
| | ^{m}C_{ds}T_{ds}Tₖₛ^{m}Cₖₛ^{m}Cₖ | | | |
| 699806 | GalNAc₃-3ₐ-_{o′}Tₖₛ | GalNAc₃- | PO | 852 |
| | Tₖₛ^{m}CₖₛA_{ds}G_{ds}T_{ds} | 3a | | |
| | ^{m}C_{ds}A_{ds}T_{ds}G_{ds}A_{ds} | | | |
| | ^{m}C_{ds}T_{ds}Tₖₛ^{m}Cₖₛ^{m}Cₖ | | | |
| 699807 | GalNAc₃-7ₐ-_{o′}Tₖₛ | GalNAc₃- | PO | 852 |
| | Tₖₛ^{m}CₖₛA_{ds}G_{ds}T_{ds} | 7a | | |
| | ^{m}C_{ds}A_{ds}T_{ds}G_{ds}A_{ds} | | | |
| | ^{m}C_{ds}T_{ds}Tₖₛ^{m}Cₖₛ^{m}Cₖ | | | |
| 699809 | GalNAc₃-7ₐ-_{o′}Tₖₛ | GalNAc₃- | PO | 852 |
| | Tₖₛ^{m}CₖₛA_{ds}G_{ds}T_{ds} | 7a | | |
| | ^{m}C_{ds}A_{ds}T_{ds}G_{ds}A_{ds} | | | |
| | ^{m}C_{ds}T_{ds}Tₑₛ^{m}Cₑₛ^{m}Cₑ | | | |
| 699811 | GalNAc₃-7ₐ-_{o′}Tₑₛ | GalNAc₃- | PO | 852 |
| | Tₑₛ^{m}CₑₛA_{ds}G_{ds}T_{ds} | 7a | | |
| | ^{m}C_{ds}A_{ds}T_{ds}G_{ds}A_{ds} | | | |
| | ^{m}C_{ds}T_{ds}Tₖₛ^{m}Cₖₛ^{m}Cₖ | | | |
| 699813 | GalNAc₃-7ₐ-_{o′}Tₖₛ | GalNAc₃- | PO | 852 |
| | T_{ds}^{m}CₖₛA_{ds}G_{ds}T_{ds} | 7a | | |
| | ^{m}C_{ds}A_{ds}T_{ds}G_{ds}A_{ds} | | | |
| | ^{m}C_{ds}T_{ds}Tₖₛ^{m}C_{ds}^{m}Cₖ | | | |
| 699815 | GalNAc₃-7ₐ-_{o′}Tₑₛ | GalNAc₃- | PO | 852 |
| | Tₖₛ^{m}CₖₛA_{ds}G_{ds}T_{ds} | 7a | | |
| | ^{m}C_{ds}A_{ds}T_{ds}G_{ds}A_{ds} | | | |
| | ^{m}C_{ds}T_{ds}Tₖₛ^{m}Cₖₛ^{m}Cₑ | | | |

The structure of GalNAc₃-3ₐ was shown previously in Example 39, and the structure of GalNAc₃-7ₐ was shown previously in Example 48. Subscripts: “e” indicates 2′-MOE modified nucleoside; “d” indicates β-D-2′-deoxyribonucleoside; “k” indicates 6′-(S)—CH₃ bicyclic nucleoside (cEt); “s” indicates phosphorothioate internucleoside linkages (PS); “o” indicates phosphodiester internucleoside linkages (PO). Supersript “m” indicates 5-methylcytosines.

### Treatment

Six to eight week old C57BL/6 mice (Jackson Laboratory, Bar Harbor, ME) were injected subcutaneously once at the dosage shown below with an oligonucleotide listed in Table 100 or with saline. Each treatment group consisted of 4 animals. The mice were sacrificed 72 hours following the final administration. Liver SRB-1 mRNA levels were measured using real-time PCR. SRB-1 mRNA levels were normalized to cyclophilin mRNA levels according to standard protocols. The results are presented as the average percent of SRB-1 mRNA levels for each treatment group relative to the saline control group. As illustrated in Table 101, treatment with antisense oligonucleotides lowered SRB-1 mRNA levels in a dose-dependent manner, and the gapmer oligonucleotides comprising a GalNAc conjugate and having wings that were either full cEt or mixed sugar modifications were significantly more potent than the parent oligonucleotide lacking a conjugate and comprising full cEt modified wings.

Body weights, liver transaminases, total bilirubin, and BUN were also measured, and the average values for each treatment group are shown in Table 101. Body weight is shown as the average percent body weight relative to the baseline body weight (% BL) measured just prior to the oligonucleotide dose.

TABLE 101
SRB-1 mRNA, ALT, AST, BUN, and total
bilirubin levels and body weights

| | | SRB-1 | | | | | |
|---|---|---|---|---|---|---|---|
| ISIS | Dosage | mRNA | ALT | AST | | | Body weight |
| No. | (mg/kg) | (% PBS) | (U/L) | (U/L) | Bil | BUN | (% BL) |
| PBS | n/a | 100 | 31 | 84 | 0.15 | 28 | 102 |
| 449093 | 1 | 111 | 18 | 48 | 0.17 | 31 | 104 |
| | 3 | 94 | 20 | 43 | 0.15 | 26 | 103 |
| | 10 | 36 | 19 | 50 | 0.12 | 29 | 104 |
| 699806 | 0.1 | 114 | 23 | 58 | 0.13 | 26 | 107 |
| | 0.3 | 59 | 21 | 45 | 0.12 | 27 | 108 |
| | 1 | 25 | 30 | 61 | 0.12 | 30 | 104 |
| 699807 | 0.1 | 121 | 19 | 41 | 0.14 | 25 | 100 |
| | 0.3 | 73 | 23 | 56 | 0.13 | 26 | 105 |
| | 1 | 24 | 22 | 69 | 0.14 | 25 | 102 |
| 699809 | 0.1 | 125 | 23 | 57 | 0.14 | 26 | 104 |
| | 0.3 | 70 | 20 | 49 | 0.10 | 25 | 105 |
| | 1 | 33 | 34 | 62 | 0.17 | 25 | 107 |
| 699811 | 0.1 | 123 | 48 | 77 | 0.14 | 24 | 106 |
| | 0.3 | 94 | 20 | 45 | 0.13 | 25 | 101 |
| | 1 | 66 | 57 | 104 | 0.14 | 24 | 107 |
| 699813 | 0.1 | 95 | 20 | 58 | 0.13 | 28 | 104 |
| | 0.3 | 98 | 22 | 61 | 0.17 | 28 | 105 |
| | 1 | 49 | 19 | 47 | 0.11 | 27 | 106 |
| 699815 | 0.1 | 93 | 30 | 79 | 0.17 | 25 | 105 |
| | 0.3 | 64 | 30 | 61 | 0.12 | 26 | 105 |
| | 1 | 24 | 18 | 41 | 0.14 | 25 | 106 |

### Example 94: Antisense Inhibition In Vivo by Oligonucleotides Targeting SRB-1 Comprising 2′-Sugar Modifications and a 5′-GalNAc₃ Conjugate

The oligonucleotides listed in Table 102 were tested in a dose-dependent study for antisense inhibition of SRB-1 in mice.

TABLE 102
Modified ASOs targeting SRB-1

| ISIS | Sequences | GalNAc₃ | | SEQ |
|---|---|---|---|---|
| No. | (5′ to 3′) | Cluster | CM | ID No. |
| 353382 | Gₑₛ^{m}CₑₛTₑₛTₑₛ^{m}Cₑₛ | n/a | n/a | 829 |
| | A_{ds}G_{ds}T_{ds}^{m}C_{ds}A_{ds} | | | |
| | T_{ds}G_{ds}A_{ds}^{m}C_{ds}T_{ds} | | | |
| | Tₑₛ^{m}Cₑₛ^{m}CₑₛTₑₛTₑ | | | |
| 700989 | GₘₛCₘₛUₘₛUₘₛCₘₛ | n/a | n/a | 853 |
| | A_{ds}G_{ds}T_{ds}^{m}C_{ds}A_{ds} | | | |
| | T_{ds}G_{ds}A_{ds}^{m}C_{ds}T_{ds} | | | |
| | UₘₛCₘₛCₘₛUₘₛUₘ | | | |
| 666904 | GalNAc₃-3ₐ-_{o′}Gₑₛ^{m}Cₑₛ | GalNAc₃- | PO | 829 |
| | TₑₛTₑₛ^{m}CₑₛA_{ds}G_{ds}T_{ds} | 3a | | |
| | ^{m}C_{ds}A_{ds}T_{ds}G_{ds}A_{ds}^{m}C_{ds} | | | |
| | T_{ds}Tₑₛ^{m}Cₑₛ^{m}CₑₛTₑₛTₑ | | | |
| 700991 | GalNAc₃-7ₐ-_{o′}GₘₛCₘₛ | GalNAc₃- | PO | 853 |
| | UₘₛUₘₛCₘₛA_{ds}G_{ds}T_{ds} | 7a | | |
| | ^{m}C_{ds}A_{ds}T_{ds}G_{ds}A_{ds}^{m}C_{ds} | | | |
| | T_{ds}UₘₛCₘₛCₘₛUₘₛUₘ | | | |

Subscript “m” indicates a 2′-O-methyl modified nucleoside. See Example 74 for complete table legend. The structure of GalNAc₃-3ₐ was shown previously in Example 39, and the structure of GalNAc₃-7ₐ was shown previously in Example 48.

### Treatment

The study was completed using the protocol described in Example 93. Results are shown in Table 103 below and show that both the 2′-MOE and 2′-OMe modified oligonucleotides comprising a GalNAc conjugate were significantly more potent than the respective parent oligonucleotides lacking a conjugate. The results of the body weights, liver transaminases, total bilirubin, and BUN measurements indicated that the compounds were all well tolerated.

TABLE 103
SRB-1 mRNA

| | Dosage | SRB-1 mRNA |
|---|---|---|
| ISIS No. | (mg/kg) | (% PBS) |
| PBS | n/a | 100 |
| 353382 | 5 | 116 |
| | 15 | 58 |
| | 45 | 27 |
| 700989 | 5 | 120 |
| | 15 | 92 |
| | 45 | 46 |
| 666904 | 1 | 98 |
| | 3 | 45 |
| | 10 | 17 |
| 700991 | 1 | 118 |
| | 3 | 63 |
| | 10 | 14 |

### Example 95: Antisense Inhibition In Vivo by Oligonucleotides Targeting SRB-1 Comprising Bicyclic Nucleosides and a 5′-GalNAc₃ Conjugate

The oligonucleotides listed in Table 104 were tested in a dose-dependent study for antisense inhibition of SRB-1 in mice.

TABLE 104
Modified ASOs targeting SRB-1

| ISIS | Sequences | GalNAc₃ | | SEQ |
|---|---|---|---|---|
| No. | (5′ to 3′) | Cluster | CM | ID No |
| 440762 | Tₖₛ^{m}CₖₛA_{ds}G_{ds}T_{ds}^{m}C_{ds}A_{ds} | n/a | n/a | 823 |
| | T_{ds}G_{ds}A_{ds}^{m}C_{ds}T_{ds}Tₖₛ^{m}Cₖ | | | |
| 666905 | GalNAc₃-3ₐ-_{o′}Tₖₛ^{m}Cₖₛ | GalNAc₃- | PO | 823 |
| | A_{ds}G_{ds}T_{ds}^{m}C_{ds}A_{ds}T_{ds}G_{ds} | 3ₐ | | |
| | A_{ds}^{m}C_{ds}T_{ds}Tₖₛ^{m}Cₖ | | | |
| 699782 | GalNAc₃-7ₐ-_{o′}Tₖₛ^{m}Cₖₛ | GalNAc₃- | PO | 823 |
| | A_{ds}G_{ds}T_{ds}^{m}C_{ds}A_{ds}T_{ds}G_{ds} | 7ₐ | | |
| | A_{ds}^{m}C_{ds}T_{ds}Tₖₛ^{m}Cₖ | | | |
| 699783 | GalNAc₃-3ₐ-_{o′}Tₗₛ^{m}Cₗₛ | GalNAc₃- | PO | 823 |
| | A_{ds}G_{ds}T_{ds}^{m}C_{ds}A_{ds}T_{ds}G_{ds} | 3ₐ | | |
| | A_{ds}^{m}C_{ds}T_{ds}Tₗₛ^{m}Cₗ | | | |
| 653621 | Tₗₛ^{m}CₗₛA_{ds}G_{ds}T_{ds}^{m}C_{ds} | GalNAc₃- | A_{d} | 824 |
| | A_{ds}T_{ds}G_{ds}A_{ds}^{m}C_{ds}T_{ds}Tₗₛₘ | 1ₐ | | |
| | CₗₒA_{do′}-GalNAc₃-1ₐ | | | |
| 439879 | T_{gs}^{m}C_{gs}A_{ds}G_{ds}T_{ds}^{m}C_{ds}A_{ds} | n/a | n/a | 823 |
| | T_{d}G_{ds}A_{ds}^{m}C_{ds}T_{ds}T_{gs}^{m}C_{g} | | | |
| 699789 | GalNAc₃-3ₐ-_{o′}T_{gs}^{m}C_{gs} | GalNAc₃- | PO | 823 |
| | A_{ds}G_{ds}T_{ds}^{m}C_{ds}A_{ds}T_{d}G_{ds} | 3ₐ | | |
| | A_{ds}^{m}C_{ds}T_{ds}T_{gs}^{m}C_{g} | | | |

Subscript g indicates a fluoro-HNA nucleoside, subscript indicates a locked nucleoside comprising a 2′-O—CH₂-4′ bridge. See the Example 74 table legend for other abbreviations. The structure of GalNAc₃-1ₐ was shown previously in Example 9, the structure of GalNAc₃-3ₐ was shown previously in Example 39, and the structure of GalNAc₃-7ₐ was shown previously in Example 48.

### Treatment

The study was completed using the protocol described in Example 93. Results are shown in Table 105 below and show that oligonucleotides comprising a GalNAc conjugate and various bicyclic nucleoside modifications were significantly more potent than the parent oligonucleotide lacking a conjugate and comprising bicyclic nucleoside modifications. Furthermore, the oligonucleotide comprising a GalNAc conjugate and fluoro-HNA modifications was significantly more potent than the parent lacking a conjugate and comprising fluoro-HNA modifications. The results of the body weights, liver transaminases, total bilirubin, and BUN measurements indicated that the compounds were all well tolerated.

TABLE 105
SRB-1 mRNA, ALT, AST, BUN, and total
bilirubin levels and body weights

| ISIS No. | Dosage (mg/kg) | SRB-1 mRNA (% PBS) |
|---|---|---|
| PBS | n/a | 100 |
| 440762 | 1 | 104 |
| | 3 | 65 |
| | 10 | 35 |
| 666905 | 0.1 | 105 |
| | 0.3 | 56 |
| | 1 | 18 |
| 699782 | 0.1 | 93 |
| | 0.3 | 63 |
| | 1 | 15 |
| 699783 | 0.1 | 105 |
| | 0.3 | 53 |
| | 1 | 12 |
| 653621 | 0.1 | 109 |
| | 0.3 | 82 |
| | 1 | 27 |
| 439879 | 1 | 96 |
| | 3 | 77 |
| | 10 | 37 |
| 699789 | 0.1 | 82 |
| | 0.3 | 69 |
| | 1 | 26 |

### Example 96: Plasma Protein Binding of Antisense Oligonucleotides Comprising a GalNAc₃ Conjugate Group

Oligonucleotides listed in Table 70 targeting ApoC-III and oligonucleotides in Table 106 targeting Apo(a) were tested in an ultra-filtration assay in order to assess plasma protein binding.

TABLE 106
Modified oligonucleotides targeting Apo(a)

| ISIS | Sequences | GalNAc₃ | | SEQ |
|---|---|---|---|---|
| No. | (5′ to 3′) | Cluster | CM | ID No |
| 494372 | TₑₛGₑₛ^{m}CₑₛTₑₛ^{m}Cₑₛ | n/a | n/a | 847 |
| | ^{m}C_{ds}G_{ds}T_{ds}T_{ds}G_{ds} | | | |
| | G_{ds}T_{ds}G_{ds}^{m}C_{ds}T_{ds} | | | |
| | TₑₛGₑₛTₑₛTₑₛ^{m}Cₑ | | | |
| 693401 | TₑₛGₑₒ^{m}CₑₒTₑₒ^{m}Cₑₒ | n/a | n/a | 847 |
| | ^{m}C_{ds}G_{ds}T_{ds}T_{ds}G_{ds} | | | |
| | G_{ds}T_{ds}G_{ds}^{m}C_{ds}T_{ds} | | | |
| | TₑₒGₑₒTₑₛTₑₛ^{m}Cₑ | | | |
| 681251 | GalNAc₃-7ₐ-_{o′}TₑₛGₑₛ | GalNAc₃- | PO | 847 |
| | ^{m}CₑₛTₑₛ^{m}Cₑₛ^{m}C_{ds}G_{ds}T_{ds} | 7ₐ | | |
| | T_{ds}G_{ds}G_{ds}T_{ds}G_{ds}^{m}C_{ds} | | | |
| | T_{ds}TₑₛGₑₛTₑₛTₑₛ^{m}Cₑ | | | |
| 681257 | GalNAc₃-7ₐ-_{o′}TₑₛGₑₒ | GalNAc₃- | PO | 847 |
| | ^{m}CₑₒTₑₒ^{m}Cₑₒ^{m}C_{ds}G_{ds}T_{ds} | 7ₐ | | |
| | T_{ds}G_{ds}G_{ds}T_{ds}G_{ds}^{m}C_{ds} | | | |
| | T_{ds}TₑₒGₑₒTₑₛTₑₛ^{m}Cₑ | | | |

See the Example 74 for table legend. The structure of GalNAc₃-7ₐ was shown previously in Example 48.

Ultrafree-MC ultrafiltration units (30,000 NMWL, low-binding regenerated cellulose membrane, Millipore, Bedford, MA) were pre-conditioned with 300 μL of 0.500 Tween 80 and centrifuged at 2000 g for 10 minutes, then with 300 μL of a 300 g/mL solution of a control oligonucleotide in H₂O and centrifuged at 2000 g for 16 minutes. In order to assess non-specific binding to the filters of each test oligonucleotide from Tables 70 and 106 to be used in the studies, 300 μL of a 250 ng/mL solution of oligonucleotide in H₂O at pH 7.4 was placed in the pre-conditioned filters and centrifuged at 2000 g for 16 minutes. The unfiltered and filtered samples were analyzed by an ELISA assay to determine the oligonucleotide concentrations. Three replicates were used to obtain an average concentration for each sample. The average concentration of the filtered sample relative to the unfiltered sample is used to determine the percent of oligonucleotide that is recovered through the filter in the absence of plasma (% recovery).

Frozen whole plasma samples collected in K3-EDTA from normal, drug-free human volunteers, cynomolgus monkeys, and CD-1 mice, were purchased from Bioreclamation LLC (Westbury, NY). The test oligonucleotides were added to 1.2 mL aliquots of plasma at two concentrations (5 and 150 μg/mL). An aliquot (300 μL) of each spiked plasma sample was placed in a pre-conditioned filter unit and incubated at 37° C. for 30 minutes, immediately followed by centrifugation at 2000 g for 16 minutes. Aliquots of filtered and unfiltered spiked plasma samples were analyzed by an ELISA to determine the oligonucleotide concentration in each sample. Three replicates per concentration were used to determine the average percentage of bound and unbound oligonucleotide in each sample. The average concentration of the filtered sample relative to the concentration of the unfiltered sample is used to determine the percent of oligonucleotide in the plasma that is not bound to plasma proteins (% unbound). The final unbound oligonucleotide values are corrected for non-specific binding by dividing the % unbound by the % recovery for each oligonucleotide. The final % bound oligonucleotide values are determined by subtracting the final % unbound values from 100. The results are shown in Table 107 for the two concentrations of oligonucleotide tested (5 and 150 μg/mL) in each species of plasma. The results show that GalNAc conjugate groups do not have a significant impact on plasma protein binding. Furthermore, oligonucleotides with full PS internucleoside linkages and mixed PO/PS linkages both bind plasma proteins, and those with full PS linkages bind plasma proteins to a somewhat greater extent than those with mixed PO/PS linkages.

TABLE 107
Percent of modified oligonucleotide bound to plasma proteins

| | Human plasma | | Monkey plasma | | Mouse plasma | |
|---|---|---|---|---|---|---|
| ISIS | 5 | 150 | 5 | 150 | 5 | 150 |
| No. | μg/mL | μg/mL | μg/mL | μg/mL | μg/mL | μg/mL |
| 304801 | 99.2 | 98.0 | 99.8 | 99.5 | 98.1 | 97.2 |
| 663083 | 97.8 | 90.9 | 99.3 | 99.3 | 96.5 | 93.0 |
| 674450 | 96.2 | 97.0 | 98.6 | 94.4 | 94.6 | 89.3 |
| 494372 | 94.1 | 89.3 | 98.9 | 97.5 | 97.2 | 93.6 |
| 693401 | 93.6 | 89.9 | 96.7 | 92.0 | 94.6 | 90.2 |
| 681251 | 95.4 | 93.9 | 99.1 | 98.2 | 97.8 | 96.1 |
| 681257 | 93.4 | 90.5 | 97.6 | 93.7 | 95.6 | 92.7 |

### Example 97: Modified Oligonucleotides Targeting TTR Comprising a GalNAc₃ Conjugate Group

The oligonucleotides shown in Table 108 comprising a GalNAc conjugate were designed to target TTR.

TABLE 108
Modified oligonucleotides targeting TTR

| ISIS | | GalNAc₃ | | SEQ |
|---|---|---|---|---|
| No. | Sequences (5′ to 3′) | Cluster | CM | ID No |
| 666941 | GalNAc₃-3_{a-o′}A_{do}Tₑₛ^{m}CₑₛTₑₛTₑₛGₑₛG_{ds}T_{ds}T_{ds}A_{ds} | GalNAc₃-3 | A_{d} | 846 |
| | ^{m}C_{ds}A_{ds}T_{ds}G_{ds}A_{ds}A_{ds}AₑₛTₑₛ^{m}Cₑₛ^{m}Cₑₛ^{m}Cₑ | | | |
| 666942 | Tₑₛ^{m}CₑₒTₑₒTₑₒGₑₒG_{ds}T_{ds}T_{ds}A_{ds}^{m}C_{ds}A_{ds}T_{ds}G_{ds} | GalNAc₃-1 | A_{d} | 843 |
| | A_{ds}A_{ds}AₑₒTₑₒ^{m}Cₑₛ^{m}Cₑₛ^{m}CₑₒA_{do′}-GalNAc₃-3ₐ | | | |
| 682876 | GalNAc₃-3_{a-o′}Tₑₛ^{m}CₑₛTₑₛTₑₛGₑₛG_{ds}T_{ds}T_{ds}A_{ds}^{m}C_{ds} | GalNAc₃-3 | PO | 842 |
| | A_{ds}T_{ds}G_{ds}A_{ds}A_{ds}AₑₛTₑₛ^{m}Cₑₛ^{m}Cₑₛ^{m}Cₑ | | | |
| 682877 | GalNAc₃-7_{a-o′}Tₑₛ^{m}CₑₛTₑₛTₑₛGₑₛG_{ds}T_{ds}T_{ds}A_{ds}^{m}C_{ds} | GalNAc₃-7 | PO | 842 |
| | A_{ds}T_{ds}G_{ds}A_{ds}A_{ds}AₑₛTₑₛ^{m}Cₑₛ^{m}Cₑₛ^{m}Cₑ | | | |
| 682878 | GalNAc₃-10_{a-o′}Tₑₛ^{m}CₑₛTₑₛTₑₛGₑₛG_{ds}T_{ds}T_{ds}A_{ds}^{m}C_{ds} | GalNAc₃-10 | PO | 842 |
| | A_{ds}T_{ds}G_{ds}A_{ds}A_{ds}AₑₛTₑₛ^{m}Cₑₛ^{m}Cₑₛ^{m}Cₑ | | | |
| 682879 | GalNAc₃-13_{a-o′}Tₑₛ^{m}CₑₛTₑₛTₑₛGₑₛG_{ds}T_{ds}T_{ds}A_{ds}^{m}C_{ds} | GalNAc₃-13 | PO | 842 |
| | A_{ds}T_{ds}G_{ds}A_{ds}A_{ds}AₑₛTₑₛ^{m}Cₑₛ^{m}Cₑₛ^{m}Cₑ | | | |
| 682880 | GalNAc₃-7_{a-o′}A_{do}Tₑₛ^{m}CₑₛTₑₛTₑₛGₑₛG_{ds}T_{ds}T_{ds}A_{ds} | GalNAc₃-7 | A_{d} | 846 |
| | ^{m}C_{ds}A_{ds}T_{ds}G_{ds}A_{ds}A_{ds}AₑₛTₑₛ^{m}Cₑₛ^{m}Cₑₛ^{m}Cₑ | | | |
| 682881 | GalNAc₃-10_{a-o′}A_{do}Tₑₛ^{m}CₑₛTₑₛTₑₛGₑₛG_{ds}T_{ds}T_{ds}A_{ds} | GalNAc₃-10 | A_{d} | 846 |
| | ^{m}C_{ds}A_{ds}T_{ds}G_{ds}A_{ds}A_{ds}AₑₛTₑₛ^{m}Cₑₛ^{m}Cₑₛ^{m}Cₑ | | | |
| 682882 | GalNAc₃-13_{a-o′}A_{do}Tₑₛ^{m}CₑₛTₑₛTₑₛGₑₛG_{ds}T_{ds}T_{ds}A_{ds} | GalNAc₃-13 | A_{d} | 846 |
| | ^{m}C_{ds}A_{ds}T_{ds}G_{ds}A_{ds}A_{ds}AₑₛTₑₛ^{m}Cₑₛ^{m}Cₑₛ^{m}Cₑ | | | |
| 684056 | Tₑₛ^{m}CₑₛTₑₛTₑₛGₑₛG_{ds}T_{ds}T_{ds}A_{ds}^{m}C_{ds}A_{ds}T_{ds}G_{ds}A_{ds} | GalNAc₃-19 | A_{d} | 846 |
| | A_{ds}AₑₛTₑₛ^{m}Cₑₛ^{m}Cₑₛ^{m}CₑₒA_{do′}-GalNAc₃-19ₐ | | | |

The legend for Table 108 can be found in Example 74. The structure of GalNAc₃-1 was shown in Example 9. The structure of GalNAc₃-3ₐ was shown in Example 39. The structure of GalNAc₃-7ₐ was shown in Example 48. The structure of GalNAc₃-10ₐ was shown in Example 46. The structure of GalNAc₃-13ₐ was shown in Example 62. The structure of GalNAc₃-19ₐ was shown in Example 70.

### Example 98: Evaluation of Pro-Inflammatory Effects of Oligonucleotides Comprising a GalNAc Conjugate in hPMBC Assay

The oligonucleotides listed in Table 109 and were tested for pro-inflammatory effects in an hPMBC assay as described in Examples 23 and 24. (See Tables 30, 83, 95, and 108 for descriptions of the oligonucleotides.) ISIS 353512 is a high responder used as a positive control, and the other oligonucleotides are described in Tables 83, 95, and 108. The results shown in Table 109 were obtained using blood from one volunteer donor. The results show that the oligonucleotides comprising mixed PO/PS internucleoside linkages produced significantly lower pro-inflammatory responses compared to the same oligonucleotides having full PS linkages. Furthermore, the GalNAc conjugate group did not have a significant effect in this assay.

TABLE 109

| ISIS No. | Eₘₐₓ/EC₅₀ | GalNAc₃ cluster | Linkages | CM |
|---|---|---|---|---|
| 353512 | 3630 | n/a | PS | n/a |
| 420915 | 802 | n/a | PS | n/a |
| 682881 | 1311 | GalNAc₃-10 | PS | A_{d} |
| 682888 | 0.26 | GalNAc₃-10 | PO/PS | A_{d} |
| 684057 | 1.03 | GalNAc₃-19 | PO/PS | A_{d} |

### Example 99: Binding Affinities of Oligonucleotides Comprising a GalNAc Conjugate for the Asialoglycoprotein Receptor

The binding affinities of the oligonucleotides listed in Table 110 (see Table 76 for descriptions of the oligonucleotides) for the asialoglycoprotein receptor were tested in a competitive receptor binding assay. The competitor ligand, al-acid glycoprotein (AGP), was incubated in 50 mM sodium acetate buffer (pH 5) with 1 U neuraminidase-agarose for 16 hours at 37° C., and >90% desialylation was confirmed by either sialic acid assay or size exclusion chromatography (SEC). Iodine monochloride was used to iodinate the AGP according to the procedure by Atsma et al. (see J Lipid Res. 1991 January; 32(1):173-81.) In this method, desialylated al-acid glycoprotein (de-AGP) was added to 10 mM iodine chloride, Na¹²⁵I, and 1 M glycine in 0.25 M NaOH. After incubation for 10 minutes at room temperature, ¹²⁵I-labeled de-AGP was separated from free ¹²⁵I by concentrating the mixture twice utilizing a 3 KDMWCO spin column. The protein was tested for labeling efficiency and purity on a HPLC system equipped with an Agilent SEC-3 column (7.8×300 mm) and a ß-RAM counter. Competition experiments utilizing ¹²⁵I-labeled de-AGP and various GalNAc-cluster containing ASOs were performed as follows. Human HepG2 cells (10⁶ cells/ml) were plated on 6-well plates in 2 ml of appropriate growth media. MEM media supplemented with 10% fetal bovine serum (FBS), 2 mM L-Glutamine and 10 mM HEPES was used. Cells were incubated 16-20 hours @37° C. with 5% and 10% CO₂ respectively. Cells were washed with media without FBS prior to the experiment. Cells were incubated for 30 min @37° C. with 1 ml competition mix containing appropriate growth media with 2% FBS, 10⁻⁸ M ¹²⁵I-labeled de-AGP and GalNAc-cluster containing ASOs at concentrations ranging from 10⁻¹¹ to 10⁻⁵ M. Non-specific binding was determined in the presence of 10⁻² M GalNAc sugar. Cells were washed twice with media without FBS to remove unbound ¹²⁵I-labeled de-AGP and competitor GalNAc ASO. Cells were lysed using Qiagen's RLT buffer containing 1% ß-mercaptoethanol. Lysates were transferred to round bottom assay tubes after a brief 10 min freeze/thaw cycle and assayed on a γ-counter. Non-specific binding was subtracted before dividing ¹²⁵I protein counts by the value of the lowest GalNAc-ASO concentration counts. The inhibition curves were fitted according to a single site competition binding equation using a nonlinear regression algorithm to calculate the binding affinities (K_{D}'s).

The results in Table 110 were obtained from experiments performed on five different days. Results for oligonucleotides marked with superscript “a” are the average of experiments run on two different days. The results show that the oligonucleotides comprising a GalNAc conjugate group on the 5′-end bound the asialoglycoprotein receptor on human HepG2 cells with 1.5 to 16-fold greater affinity than the oligonucleotides comprising a GalNAc conjugate group on the 3′-end.

TABLE 110
Asialoglycoprotein receptor binding assay results

| | | Oligonucleotide end | |
|---|---|---|---|
| ISIS | | to which GalNAc | K_{D} |
| No. | GalNAc conjugate | conjugate is attached | (nM) |
| 661161^{a} | GalNAc₃-3 | 5′ | 3.7 |
| 666881^{a} | GalNAc₃-10 | 5′ | 7.6 |
| 666981 | GalNAc₃-7 | 5′ | 6.0 |
| 670061 | GalNAc₃-13 | 5′ | 7.4 |
| 655861^{a} | GalNAc₃-1 | 3′ | 11.6 |
| 677841^{a} | GalNAc₃-19 | 3′ | 60.8 |

### Example 100: Antisense Inhibition In Vivo by Oligonucleotides Comprising a GalNAc Conjugate Group Targeting Apo(a) In Vivo

The oligonucleotides listed in Table 111a below were tested in a single dose study for duration of action in mice.

TABLE 111a
Modified ASOs targeting APO(a)

| ISIS | | GalNAc₃ | | SEQ |
|---|---|---|---|---|
| No. | Sequences (5′ to 3′) | Cluster | CM | ID No. |
| 681251 | GalNAc₃-7ₐ-_{o′}TₑₛGₑₛ^{m}CₑₛTₑₛ^{m}Cₑₛ^{m}C_{ds}G_{ds}T_{ds}T_{ds}G_{ds}G_{ds} | GalNAc₃-7a | PO | 847 |
| | T_{ds}G_{ds}^{m}C_{ds}T_{ds}TₑₛGₑₛTₑₛTₑₛ^{m}Cₑ | | | |
| 681257 | GalNAc₃-7ₐ-_{o′}TₑₛGₑₒ^{m}CₑₒTₑₒ^{m}Cₑₒ^{m}C_{ds}G_{ds}T_{ds}T_{ds}G_{ds}G_{ds} | GalNAc₃-7a | PO | 847 |
| | T_{ds}G_{ds}^{m}C_{ds}T_{ds}TₑₒGₑₒTₑₛTₑₛ^{m}Cₑ | | | |

The structure of GalNAc₃-7ₐ was shown in Example 48.

### Treatment

Female transgenic mice that express human Apo(a) were each injected subcutaneously once per week, for a total of 6 doses, with an oligonucleotide and dosage listed in Table 111b or with PBS. Each treatment group consisted of 3 animals. Blood was drawn the day before dosing to determine baseline levels of Apo(a) protein in plasma and at 72 hours, 1 week, and 2 weeks following the first dose. Additional blood draws will occur at 3 weeks, 4 weeks, 5 weeks, and 6 weeks following the first dose. Plasma Apo(a) protein levels were measured using an ELISA. The results in Table 111b are presented as the average percent of plasma Apo(a) protein levels for each treatment group, normalized to baseline levels (% BL), The results show that the oligonucleotides comprising a GalNAc conjugate group exhibited potent reduction in Apo(a) expression. This potent effect was observed for the oligonucleotide that comprises full PS internucleoside linkages and the oligonucleotide that comprises mixed PO and PS linkages.

TABLE 111b
Apo(a) plasma protein levels

| | | Apo(a) at | Apo(a) at | Apo(a) at |
|---|---|---|---|---|
| ISIS | Dosage | 72 hours | 1 week | 3 weeks |
| No. | (mg/kg) | (% BL) | (% BL) | (% BL) |
| PBS | n/a | 116 | 104 | 107 |
| 681251 | 0.3 | 97 | 108 | 93 |
| | 1.0 | 85 | 77 | 57 |
| | 3.0 | 54 | 49 | 11 |
| | 10.0 | 23 | 15 | 4 |
| 681257 | 0.3 | 114 | 138 | 104 |
| | 1.0 | 91 | 98 | 54 |
| | 3.0 | 69 | 40 | 6 |
| | 10.0 | 30 | 21 | 4 |

### Example 101: Antisense Inhibition by Oligonucleotides Comprising a GalNAc Cluster Linked Via a Stable Moiety

The oligonucleotides listed in Table 112 were tested for inhibition of mouse APOC-III expression in vivo. C57Bl/6 mice were each injected subcutaneously once with an oligonucleotide listed in Table 112 or with PBS. Each treatment group consisted of 4 animals. Each mouse treated with ISIS 440670 received a dose of 2, 6, 20, or 60 mg/kg. Each mouse treated with ISIS 680772 or 696847 received 0.6, 2, 6, or 20 mg/kg. The GalNAc conjugate group of ISIS 696847 is linked via a stable moiety, a phosphorothioate linkage instead of a readily cleavable phosphodiester containing linkage. The animals were sacrificed 72 hours after the dose. Liver APOC-III mRNA levels were measured using real-time PCR. APOC-III mRNA levels were normalized to cyclophilin mRNA levels according to standard protocols. The results are presented in Table 112 as the average percent of APOC-III mRNA levels for each treatment group relative to the saline control group. The results show that the oligonucleotides comprising a GalNAc conjugate group were significantly more potent than the oligonucleotide lacking a conjugate group. Furthermore, the oligonucleotide comprising a GalNAc conjugate group linked to the oligonucleotide via a cleavable moiety (ISIS 680772) was even more potent than the oligonucleotide comprising a GalNAc conjugate group linked to the oligonucleotide via a stable moiety (ISIS 696847).

TABLE 112
Modified oligonucleotides targeting mouse APOC-III

| | | | | APOC-III | SEQ |
|---|---|---|---|---|---|
| ISIS | | | Dosage | mRNA | ID |
| No. | Sequences (5′ to 3′) | CM | (mg/kg) | (% PBS) | No. |
| 440670 | ^{m}CₑₛAₑₛGₑₛ^{m}CₑₛTₑₛT_{ds}T_{ds}A_{ds}T_{ds}T_{ds}A_{ds} | n/a | 2 | 92 | 849 |
| | G_{ds}G_{ds}G_{ds}A_{ds}^{m}CₑₛAₑₛGₑₛ^{m}CₑₛAₑ | | 6 | 86 | |
| | | | 20 | 59 | |
| | | | 60 | 37 | |
| 680772 | GalNAc₃-7_{a-o′}^{m}CₑₛAₑₛGₑₛ^{m}CₑₛTₑₛT_{ds}T_{ds}A_{ds} | PO | 0.6 | 79 | 849 |
| | T_{ds}T_{ds}A_{ds}G_{ds}G_{ds}G_{ds}A_{ds}^{m}CₑₛAₑₛGₑₛ^{m}CₑₛAₑ | | 2 | 58 | |
| | | | 6 | 31 | |
| | | | 20 | 13 | |
| 696847 | GalNAc₃-7_{a-s′} | n/a | 0.6 | 83 | 849 |
| | ^{m}CₑₛAₑₛGₑₛ^{m}CₑₛTₑₛT_{ds}T_{ds}A_{ds}T_{ds} | (PS) | 2 | 73 | |
| | T_{ds}A_{ds}G_{ds}G_{ds}G_{ds}A_{ds}^{m}CₑₛAₑₛGₑₛ^{m}CₑₛAₑ | | 6 | 40 | |
| | | | 20 | 28 | |

The structure of GalNAc₃-7ₐ was shown in Example 48.

### Example 102: Distribution in Liver of Antisense Oligonucleotides Comprising a GalNAc Conjugate

The liver distribution of ISIS 353382 (see Table 36) that does not comprise a GalNAc conjugate and ISIS 655861 (see Table 36) that does comprise a GalNAc conjugate was evaluated. Male balb/c mice were subcutaneously injected once with ISIS 353382 or 655861 at a dosage listed in Table 113. Each treatment group consisted of 3 animals except for the 18 mg/kg group for ISIS 655861, which consisted of 2 animals. The animals were sacrificed 48 hours following the dose to determine the liver distribution of the oligonucleotides. In order to measure the number of antisense oligonucleotide molecules per cell, a Ruthenium (II) tris-bipyridine tag (MSD TAG, Meso Scale Discovery) was conjugated to an oligonucleotide probe used to detect the antisense oligonucleotides. The results presented in Table 113 are the average concentrations of oligonucleotide for each treatment group in units of millions of oligonucleotide molecules per cell. The results show that at equivalent doses, the oligonucleotide comprising a GalNAc conjugate was present at higher concentrations in the total liver and in hepatocytes than the oligonucleotide that does not comprise a GalNAc conjugate. Furthermore, the oligonucleotide comprising a GalNAc conjugate was present at lower concentrations in non-parenchymal liver cells than the oligonucleotide that does not comprise a GalNAc conjugate. And while the concentrations of ISIS 655861 in hepatocytes and non-parenchymal liver cells were similar per cell, the liver is approximately 80% hepatocytes by volume. Thus, the majority of the ISIS 655861 oligonucleotide that was present in the liver was found in hepatocytes, whereas the majority of the ISIS 353382 oligonucleotide that was present in the liver was found in non-parenchymal liver cells.

TABLE 113

| | | | | Concentration |
|---|---|---|---|---|
| | | Concentration | Concentration | in non-parenchymal |
| | | in whole liver | in hepatocytes | liver cells |
| ISIS | Dosage | (molecules*10\hat{ }6 | (molecules*10\hat{ }6 | (molecules*10\hat{ }6 |
| No. | (mg/kg) | per cell) | per cell) | per cell) |
| 353382 | 3 | 9.7 | 1.2 | 37.2 |
| | 10 | 17.3 | 4.5 | 34.0 |
| | 20 | 23.6 | 6.6 | 65.6 |
| | 30 | 29.1 | 11.7 | 80.0 |
| | 60 | 73.4 | 14.8 | 98.0 |
| | 90 | 89.6 | 18.5 | 119.9 |
| 655861 | 0.5 | 2.6 | 2.9 | 3.2 |
| | 1 | 6.2 | 7.0 | 8.8 |
| | 3 | 19.1 | 25.1 | 28.5 |
| | 6 | 44.1 | 48.7 | 55.0 |
| | 18 | 76.6 | 82.3 | 77.1 |

### Example 103: Duration of Action In Vivo of Oligonucleotides Targeting APOC-III Comprising a GalNAc₃ Conjugate

The oligonucleotides listed in Table 114 below were tested in a single dose study for duration of action in mice.

TABLE 114
Modified ASOs targeting APOC-III

| ISIS | | GalNAc₃ | | SEQ |
|---|---|---|---|---|
| No. | Sequences (5′ to 3′) | Cluster | CM | ID No. |
| 304801 | AₑₛGₑₛ^{m}CₑₛTₑₛTₑₛ^{m}C_{ds}T_{ds}T_{ds}G_{ds}T_{ds}^{m}C_{ds}^{m}C_{ds}A_{ds}G_{ds}^{m}C_{ds}TₑₛTₑₛ | n/a | n/a | 821 |
| | TₑₛAₑₛTₑ | | | |
| 663084 | GalNAc₃-3ₐ-_{o′}A_{do}AₑₛGₑₒ^{m}CₑₒTₑₒTₑₒ^{m}C_{ds}T_{ds}T_{ds}G_{ds}T_{ds}^{m}C_{ds} | GalNAc₃-3a | A_{d} | 837 |
| | ^{m}C_{ds}A_{ds}G_{ds}^{m}C_{ds}TₑₒTₑₒTₑₛAₑₛTₑ | | | |
| 679241 | AₑₛGₑₒ^{m}CₑₒTₑₒTₑₒ^{m}C_{ds}T_{ds}T_{ds}G_{ds}T_{ds}^{m}C_{ds}^{m}C_{ds}A_{ds}G_{ds}^{m}C_{ds}TₑₒTₑₒ | GalNAc₃-19a | A_{d} | 822 |
| | TₑₛAₑₛTₑₒA_{do′}-GalNAc₃-19ₐ | | | |

The structure of GalNAc₃-3ₐ was shown in Example 39, and GalNAc₃-19ₐ was shown in Example 70.

### Treatment

Female transgenic mice that express human APOC-III were each injected subcutaneously once with an oligonucleotide listed in Table 114 or with PBS. Each treatment group consisted of 3 animals. Blood was drawn before dosing to determine baseline and at 3, 7, 14, 21, 28, 35, and 42 days following the dose. Plasma triglyceride and APOC-III protein levels were measured as described in Example 20. The results in Table 115 are presented as the average percent of plasma triglyceride and APOC-III levels for each treatment group, normalized to baseline levels. A comparison of the results in Table 71 of example 79 with the results in Table 115 below show that oligonucleotides comprising a mixture of phosphodiester and phosphorothioate internucleoside linkages exhibited increased duration of action than equivalent oligonucleotides comprising only phosphorothioate internucleoside linkages.

TABLE 115
Plasma triglyceride and APOC-III protein levels in transgenic mice

| | | Time point | | APOC-III | | |
|---|---|---|---|---|---|---|
| ISIS | Dosage | (days post- | Triglycerides | protein | GalNAc₃ | |
| No. | (mg/kg) | dose) | (% baseline) | (% baseline) | Cluster | CM |
| PBS | n/a | 3 | 96 | 101 | n/a | n/a |
| | | 7 | 88 | 98 | | |
| | | 14 | 91 | 103 | | |
| | | 21 | 69 | 92 | | |
| | | 28 | 83 | 81 | | |
| | | 35 | 65 | 86 | | |
| | | 42 | 72 | 88 | | |
| 304801 | 30 | 3 | 42 | 46 | n/a | n/a |
| | | 7 | 42 | 51 | | |
| | | 14 | 59 | 69 | | |
| | | 21 | 67 | 81 | | |
| | | 28 | 79 | 76 | | |
| | | 35 | 72 | 95 | | |
| | | 42 | 82 | 92 | | |
| 663084 | 10 | 3 | 35 | 28 | GalNAc₃-3a | A_{d} |
| | | 7 | 23 | 24 | | |
| | | 14 | 23 | 26 | | |
| | | 21 | 23 | 29 | | |
| | | 28 | 30 | 22 | | |
| | | 35 | 32 | 36 | | |
| | | 42 | 37 | 47 | | |
| 679241 | 10 | 3 | 38 | 30 | GalNAc₃-19a | A_{d} |
| | | 7 | 31 | 28 | | |
| | | 14 | 30 | 22 | | |
| | | 21 | 36 | 34 | | |
| | | 28 | 48 | 34 | | |
| | | 35 | 50 | 45 | | |
| | | 42 | 72 | 64 | | |

### Example 104: Synthesis of Oligonucleotides Comprising a 5′-GalNAc₂ Conjugate

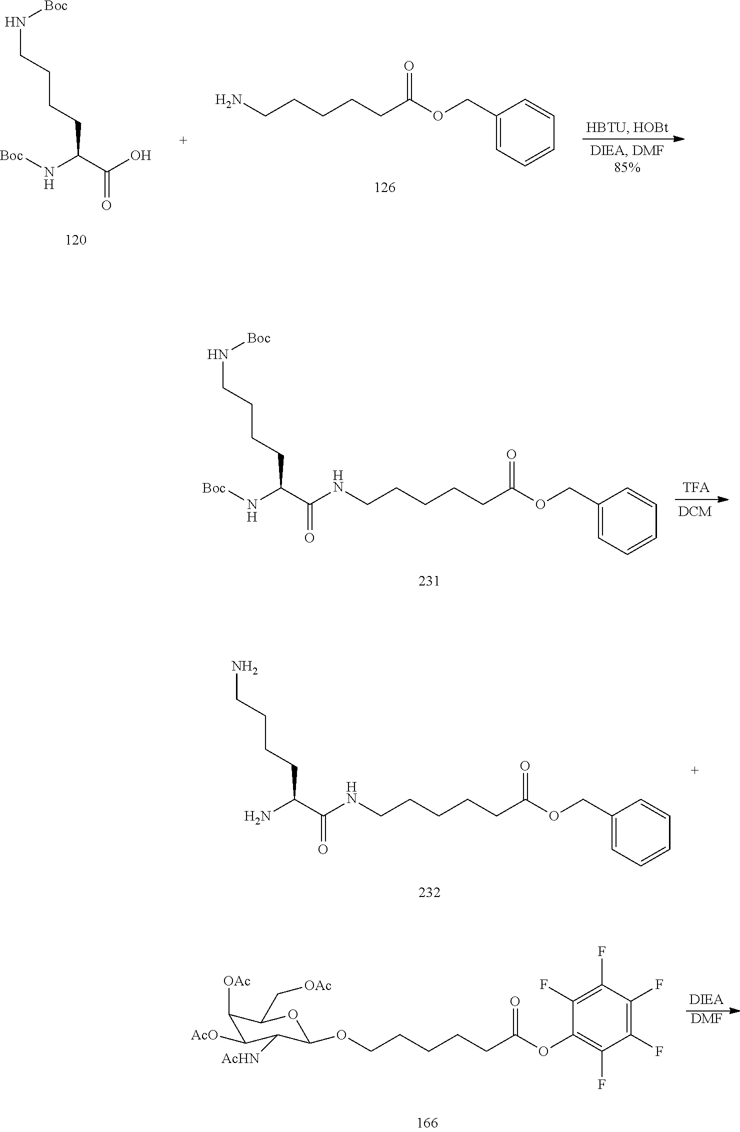

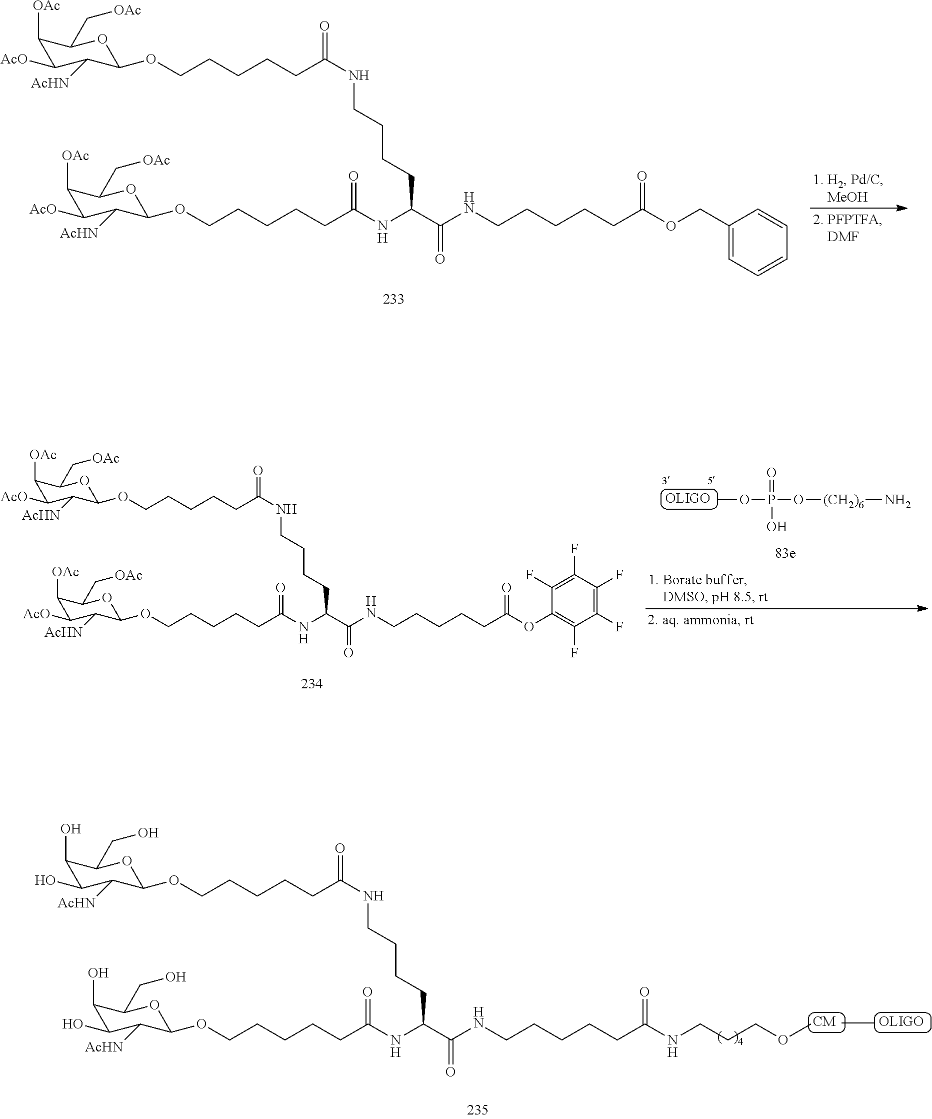

Compound 120 is commercially available, and the synthesis of compound 126 is described in Example 49. Compound 120 (1 g, 2.89 mmol), HBTU (0.39 g, 2.89 mmol), and HOBt (1.64 g, 4.33 mmol) were dissolved in DMF (10 mL. and N,N-diisopropylethylamine (1.75 mL, 10.1 mmol) were added. After about 5 min, aminohexanoic acid benzyl ester (1.36 g, 3.46 mmol) was added to the reaction. After 3 h, the reaction mixture was poured into 100 mL of 1 M NaHSO₄ and extracted with 2×50 mL ethyl acetate. Organic layers were combined and washed with 3×40 mL sat NaHCO₃ and 2×brine, dried with Na₂SO₄, filtered and concentrated. The product was purified by silica gel column chromatography (DCM:EA:Hex, 1:1:1) to yield compound 231. LCMS and NMR were consistent with the structure. Compounds 231 (1.34 g, 2.438 mmol) was dissolved in dichloromethane (10 mL) and trifluoracetic acid (10 mL) was added. After stirring at room temperature for 2 h, the reaction mixture was concentrated under reduced pressure and co-evaporated with toluene (3×10 mL). The residue was dried under reduced pressure to yield compound 232 as the trifuloracetate salt. The synthesis of compound 166 is described in Example 54. Compound 166 (3.39 g, 5.40 mmol) was dissolved in DMF (3 mL). A solution of compound 232 (1.3 g, 2.25 mmol) was dissolved in DMF (3 mL) and N,N-diisopropylethylamine (1.55 mL) was added. The reaction was stirred at room temperature for 30 minutes, then poured into water (80 mL) and the aqueous layer was extracted with EtOAc (2×100 mL). The organic phase was separated and washed with sat. aqueous NaHCO₃ (3×80 mL), 1 M NaHSO₄ (3×80 mL) and brine (2×80 mL), then dried (Na₂SO₄), filtered, and concentrated.

The residue was purified by silica gel column chromatography to yield compound 233. LCMS and NMR were consistent with the structure. Compound 233 (0.59 g, 0.48 mmol) was dissolved in methanol (2.2 mL) and ethyl acetate (2.2 mL). Palladium on carbon (10 wt % Pd/C, wet, 0.07 g) was added, and the reaction mixture was stirred under hydrogen atmosphere for 3 h. The reaction mixture was filtered through a pad of Celite and concentrated to yield the carboxylic acid. The carboxylic acid (1.32 g, 1.15 mmol, cluster free acid) was dissolved in DMF (3.2 mL). To this NN-diisopropylehtylamine (0.3 mL, 1.73 mmol) and PFPTFA (0.30 mL, 1.73 mmol) were added. After 30 min stirring at room temperature the reaction mixture was poured into water (40 mL) and extracted with EtOAc (2×50 mL). A standard work-up was completed as described above to yield compound 234. LCMS and NMR were consistent with the structure. Oligonucleotide 235 was prepared using the general procedure described in Example 46. The GalNAc2 cluster portion (GalNAc₂-24ₐ) of the conjugate group GalNAc₂-24 can be combined with any cleavable moiety present on the oligonucleotide to provide a variety of conjugate groups. The structure of GalNAc₂-24 (GalNAc₂-24ₐ-CM) is shown below:

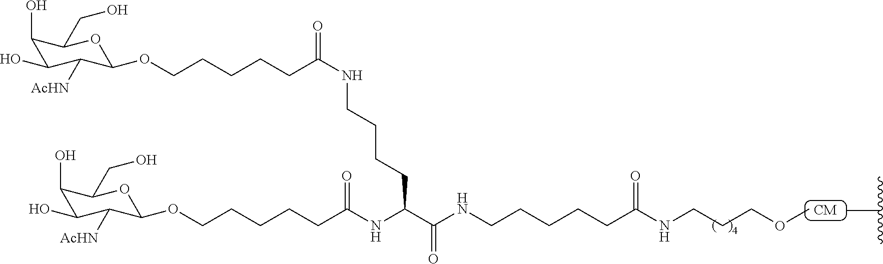

### Example 105: Synthesis of oligonucleotides comprising a GalNAc₁-25 conjugate

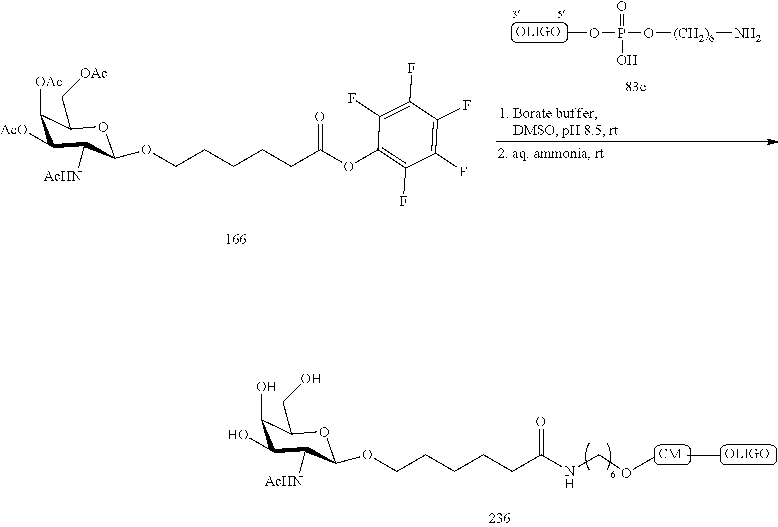

The synthesis of compound 166 is described in Example 54. Oligonucleotide 236 was prepared using the general procedure described in Example 46.

Alternatively, oligonucleotide 236 was synthesized using the scheme shown below, and compound 238 was used to form the oligonucleotide 236 using procedures described in Example 10.

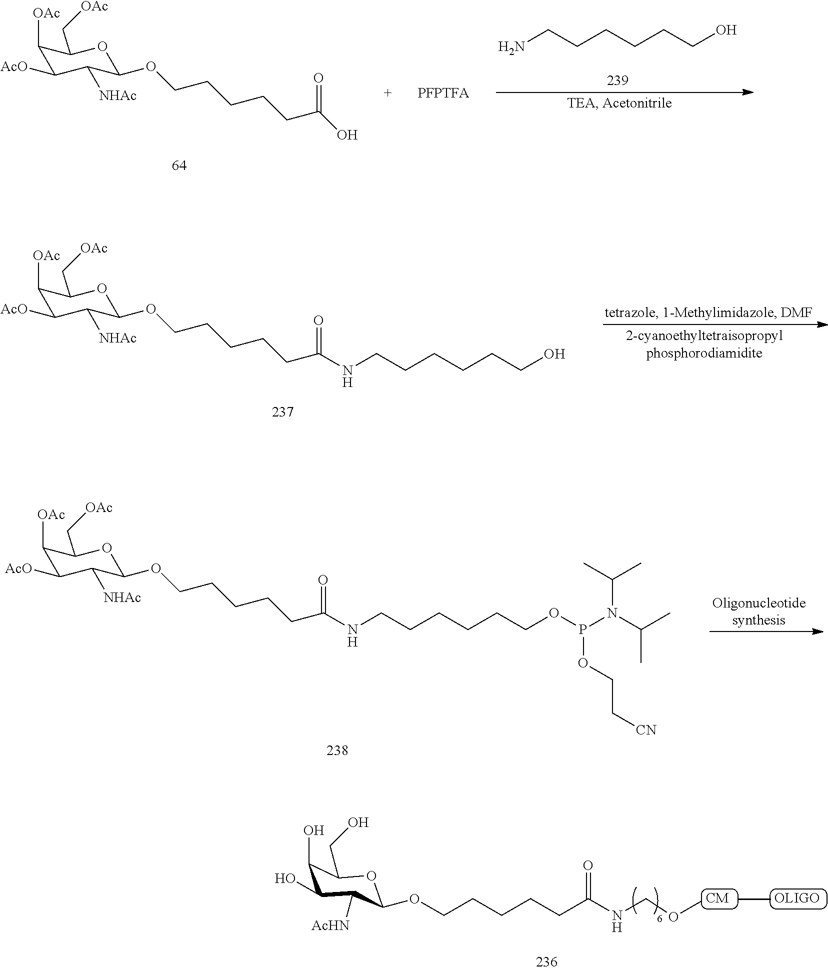

The GalNAc₁ cluster portion (GalNAc₁-25ₐ) of the conjugate group GalNAc₁-25 can be combined with any cleavable moiety present on the oligonucleotide to provide a variety of conjugate groups. The structure of GalNAc₁-25 (GalNAc₁-25ₐ-CM) is shown below:

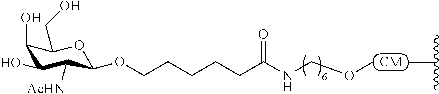

### Example 106: Antisense Inhibition In Vivo by Oligonucleotides Targeting SRB-1 Comprising a 5′-GalNAc₂ or a 5′-GalNAc₃ Conjugate

Oligonucleotides listed in Tables 116 and 117 were tested in dose-dependent studies for antisense inhibition of SRB-1 in mice.

### Treatment

Six to week old, male C57BL/6 mice (Jackson Laboratory, Bar Harbor, ME) were injected subcutaneously once with 2, 7, or 20 mg/kg of ISIS No. 440762; or with 0.2, 0.6, 2, 6, or 20 mg/kg of ISIS No. 686221, 686222, or 708561; or with saline. Each treatment group consisted of 4 animals. The mice were sacrificed 72 hours following the final administration. Liver SRB-1 mRNA levels were measured using real-time PCR. SRB-1 mRNA levels were normalized to cyclophilin mRNA levels according to standard protocols. The antisense oligonucleotides lowered SRB-1 mRNA levels in a dose-dependent manner, and the ED₅₀ results are presented in Tables 116 and 117. Although previous studies showed that trivalent GalNAc-conjugated oligonucleotides were significantly more potent than divalent GalNAc-conjugated oligonucleotides, which were in turn significantly more potent than monovalent GalNAc conjugated oligonucleotides (see, e.g., Khorev et al., Bioorg. & Med. Chem., Vol. 16, 5216-5231 (2008)), treatment with antisense oligonucleotides comprising monovalent, divalent, and trivalent GalNAc clusters lowered SRB-1 mRNA levels with similar potencies as shown in Tables 116 and 117.

TABLE 116
Modified oligonucleotides targeting SRB-1

| | | | | SEQ |
|---|---|---|---|---|
| ISIS | | GalNAc | ED₅₀ | ID |
| No. | Sequences (5′ to 3′) | Cluster | (mg/kg) | No |
| 440762 | Tₖₛ^{m}CₖₛA_{ds}G_{ds}T_{ds}^{m}C_{ds}A_{ds}T_{ds}G_{ds}A_{ds}^{m}C_{ds}T_{ds}Tₖₛ^{m}Cₖ | n/a | 4.7 | 823 |
| 686221 | GalNAc₂-24ₐ-_{o′}A_{do}Tₖₛ^{m}CₖₛA_{ds}G_{ds}T_{ds}^{m}C_{ds}A_{ds}T_{ds}G_{ds}A_{ds} | GalNAc₂-24ₐ | 0.39 | 827 |
| | ^{m}C_{ds}T_{ds}Tₖₛ^{m}Cₖ | | | |
| 686222 | GalNAc₃-13ₐ-_{o′}A_{do}Tₖₛ^{m}CₖₛA_{ds}G_{ds}T_{ds}^{m}C_{ds}A_{ds}T_{ds}G_{ds}A_{ds} | GalNAc₃-13ₐ | 0.41 | 827 |
| | ^{m}C_{ds}T_{ds}Tₖₛ^{m}Cₖ | | | |

See Example 93 for table legend. The structure of GalNAc₃-13ₐ was shown in Example 62, and the structure of GalNAc₂-24ₐ was shown in Example 104.

TABLE 117
Modified oligonucleotides targeting SRB-1

| | | | | SEQ |
|---|---|---|---|---|
| ISIS | | GalNAc | ED₅₀ | ID |
| No. | Sequences (5′ to 3′) | Cluster | (mg/kg) | No |
| 440762 | Tₖₛ^{m}CₖₛA_{ds}G_{ds}T_{ds}^{m}C_{ds}A_{ds}T_{ds}G_{ds}A_{ds}^{m}C_{ds}T_{ds}Tₖₛ^{m}Cₖ | n/a | 5 | 823 |
| 708561 | GalNAc₁-25ₐ-_{o′}Tₖₛ^{m}CₖₛA_{ds}G_{ds}T_{ds}^{m}C_{ds}A_{ds}T_{ds}G_{ds}A_{ds} | GalNAc₁-25ₐ | 0.4 | 823 |
| | ^{m}C_{ds}T_{ds}Tₖₛ^{m}Cₖ | | | |

See Example 93 for table legend. The structure of GalNAc₁-25ₐ was shown in Example 105.

The concentrations of the oligonucleotides in Tables 116 and 117 in liver were also assessed, using procedures described in Example 75. The results shown in Tables 117a and 117b below are the average total antisense oligonucleotide tissues levels for each treatment group, as measured by UV in units of μg oligonucleotide per gram of liver tissue. The results show that the oligonucleotides comprising a GalNAc conjugate group accumulated in the liver at significantly higher levels than the same dose of the oligonucleotide lacking a GalNAc conjugate group. Furthermore, the antisense oligonucleotides comprising one, two, or three GalNAc ligands in their respective conjugate groups all accumulated in the liver at similar levels. This result is surprising in view of the Khorev et al. literature reference cited above and is consistent with the activity data shown in Tables 116 and 117 above.

TABLE 117a
Liver concentrations of oligonucleotides comprising
a GalNAc₂ or GalNAc₃ conjugate group

| ISIS | Dosage | [Antisense oligonucleotide] | | |
|---|---|---|---|---|
| No. | (mg/kg) | (μg/g) | GalNAc cluster | CM |
| 440762 | 2 | 2.1 | n/a | n/a |
| | 7 | 13.1 | | |
| | 20 | 31.1 | | |
| 686221 | 0.2 | 0.9 | GalNAc₂-24a | A_{d} |
| | 0.6 | 2.7 | | |
| | 2 | 12.0 | | |
| | 6 | 26.5 | | |
| 686222 | 0.2 | 0.5 | GalNAc₃-13a | A_{d} |
| | 0.6 | 1.6 | | |
| | 2 | 11.6 | | |
| | 6 | 19.8 | | |

TABLE 117b
Liver concentrations of oligonucleotides
comprising a GalNAc₁ conjugate group

| ISIS | Dosage | [Antisense oligonucleotide] | | |
|---|---|---|---|---|
| No. | (mg/kg) | (μg/g) | GalNAc cluster | CM |
| 440762 | 2 | 2.3 | n/a | n/a |
| | 7 | 8.9 | | |
| | 20 | 23.7 | | |
| 708561 | 0.2 | 0.4 | GalNAc₁-25ₐ | PO |
| | 0.6 | 1.1 | | |
| | 2 | 5.9 | | |
| | 6 | 23.7 | | |
| | 20 | 53.9 | | |

### Example 107: Synthesis of Oligonucleotides Comprising a GalNAc₁-26 or GalNAc₁-27 Conjugate

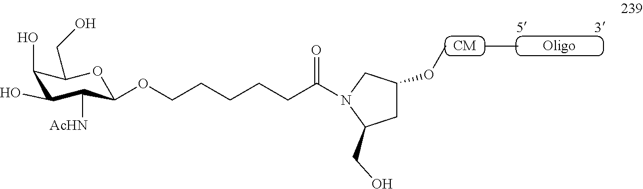

Oligonucleotide 239 is synthesized via coupling of compound 47 (see Example 15) to acid 64 (see Example 32) using HBTU and DIEA in DMF. The resulting amide containing compound is phosphitylated, then added to the 5′-end of an oligonucleotide using procedures described in Example 10. The GalNAc₁ cluster portion (GalNAc₁-26ₐ) of the conjugate group GalNAc₁-26 can be combined with any cleavable moiety present on the oligonucleotide to provide a variety of conjugate groups. The structure of GalNAc₁-26 (GalNAc₁-26ₐ-CM) is shown below:

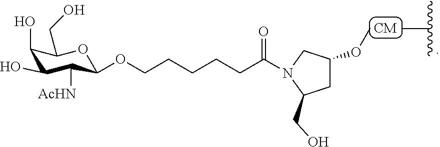

In order to add the GalNAc₁ conjugate group to the 3′-end of an oligonucleotide, the amide formed from the reaction of compounds 47 and 64 is added to a solid support using procedures described in Example 7. The oligonucleotide synthesis is then completed using procedures described in Example 9 in order to form oligonucleotide 240.

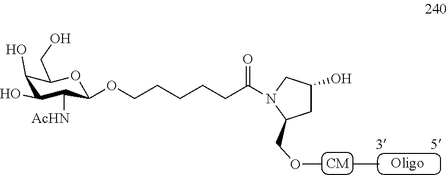

The GalNAc₁ cluster portion (GalNAc₁-27ₐ) of the conjugate group GalNAc₁-27 can be combined with any cleavable moiety present on the oligonucleotide to provide a variety of conjugate groups. The structure of GalNAc₁-27 (GalNAc₁-27ₐ-CM) is shown below:

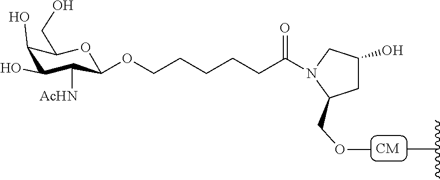

### Example 108: Antisense Inhibition In Vivo by Oligonucleotides Comprising a GalNAc Conjugate Group Targeting Apo(a) In Vivo

The oligonucleotides listed in Table 118 below were tested in a single dose study in mice.

TABLE 118
Modified ASOs targeting APO(a)

| ISIS | | GalNAc₃ | | SEQ |
|---|---|---|---|---|
| No. | Sequences (5′ to 3′) | Cluster | CM | ID No. |
| 494372 | TₑₛGₑₛ^{m}CₑₛTₑₛ^{m}Cₑₛ^{m}C_{ds}G_{ds}T_{ds}T_{ds}G_{ds}G_{ds}T_{ds}G_{ds}^{m}C_{ds} | n/a | n/a | 847 |
| | T_{ds}TₑₛGₑₛTₑₛTₑₛ^{m}Cₑ | | | |
| 681251 | GalNAc₃-7ₐ-_{o′}TₑₛGₑₛ^{m}CₑₛTₑₛ^{m}Cₑₛ^{m}C_{ds}G_{ds}T_{ds}T_{ds}G_{ds}G_{ds} | GalNAc₃-7a | PO | 847 |
| | T_{ds}G_{ds}^{m}C_{ds}T_{ds}TₑₛGₑₛTₑₛTₑₛ^{m}Cₑ | | | |
| 681255 | GalNAc₃-3ₐ-_{o′}TₑₛGₑₒ^{m}CₑₒTₑₒ^{m}Cₑₒ^{m}C_{ds}G_{ds}T_{ds}T_{ds}G_{ds}G_{ds} | GalNAc₃-3a | PO | 847 |
| | T_{ds}G_{ds}^{m}C_{ds}T_{ds}TₑₒGₑₒTₑₛTₑₛ^{m}Cₑ | | | |
| 681256 | GalNAc₃-10ₐ-_{o′}TₑₛGₑₒ^{m}CₑₒTₑₒ^{m}Cₑₒ^{m}C_{ds}G_{ds}T_{ds}T_{ds}G_{ds}G_{ds} | GalNAc₃-10a | PO | 847 |
| | T_{ds}G_{ds}^{m}C_{ds}T_{ds}TₑₒGₑₒTₑₛTₑₛ^{m}Cₑ | | | |
| 681257 | GalNAc₃-7ₐ-_{o′}TₑₛGₑₒ^{m}CₑₒTₑₒ^{m}Cₑₒ^{m}C_{ds}G_{ds}T_{ds}T_{ds}G_{ds}G_{ds} | GalNAc₃-7a | PO | 847 |
| | T_{ds}G_{ds}^{m}C_{ds}T_{ds}TₑₒGₑₒTₑₛTₑₛ^{m}Cₑ | | | |
| 681258 | GalNAc₃-13ₐ-_{o′}TₑₛGₑₒ^{m}CₑₒTₑₒ^{m}Cₑₒ^{m}C_{ds}G_{ds}T_{ds}T_{ds}G_{ds}G_{ds} | GalNAc₃-13a | PO | 847 |
| | T_{ds}G_{ds}^{m}C_{ds}T_{ds}TₑₒGₑₒTₑₛTₑₛ^{m}Cₑ | | | |
| 681260 | TₑₛGₑₒ^{m}CₑₒTₑₒ^{m}Cₑₒ^{m}C_{ds}G_{ds}T_{ds}T_{ds}G_{ds}G_{ds}T_{ds}G_{ds}^{m}C_{ds}T_{ds}TₑₒGₑₒ | GalNAc₃-19a | A_{d} | 854 |
| | TₑₛTₑₛ^{m}CₑₒA_{do′}-GalNAc₃-19 | | | |

The structure of GalNAc₃-7ₐ was shown in Example 48.

### Treatment

Male transgenic mice that express human Apo(a) were each injected subcutaneously once with an oligonucleotide and dosage listed in Table 119 or with PBS. Each treatment group consisted of 4 animals. Blood was drawn the day before dosing to determine baseline levels of Apo(a) protein in plasma and at 1 week following the first dose. Additional blood draws will occur weekly for approximately 8 weeks. Plasma Apo(a) protein levels were measured using an ELISA. The results in Table 119 are presented as the average percent of plasma Apo(a) protein levels for each treatment group, normalized to baseline levels (% BL), The results show that the antisense oligonucleotides reduced Apo(a) protein expression. Furthermore, the oligonucleotides comprising a GalNAc conjugate group exhibited even more potent reduction in Apo(a) expression than the oligonucleotide that does not comprise a conjugate group.

TABLE 119
Apo(a) plasma protein levels

| ISIS No. | Dosage (mg/kg) | Apo(a) at 1 week (% BL) |
|---|---|---|
| PBS | n/a | 143 |
| 494372 | 50 | 58 |
| 681251 | 10 | 15 |
| 681255 | 10 | 14 |
| 681256 | 10 | 17 |
| 681257 | 10 | 24 |
| 681258 | 10 | 22 |
| 681260 | 10 | 26 |

### Example 109: Synthesis of Oligonucleotides Comprising a GalNAc₁-28 or GalNAc₁-29 Conjugate

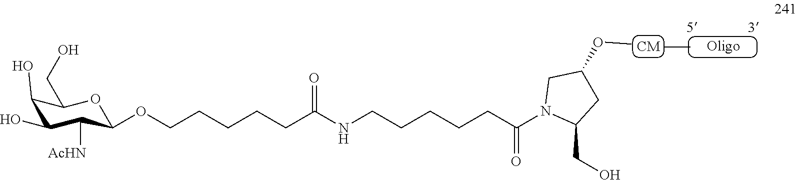

Oligonucleotide 241 is synthesized using procedures similar to those described in Example 71 to form the phosphoramidite intermediate, followed by procedures described in Example 10 to synthesize the oligonucleotide. The GalNAc₁ cluster portion (GalNAc₁-28ₐ) of the conjugate group GalNAc₁-28 can be combined with any cleavable moiety present on the oligonucleotide to provide a variety of conjugate groups. The structure of GalNAc₁-28 (GalNAc₁-28ₐ-CM) is shown below:

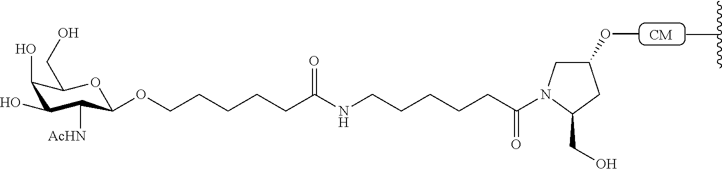

In order to add the GalNAc₁ conjugate group to the 3′-end of an oligonucleotide, procedures similar to those described in Example 71 are used to form the hydroxyl intermediate, which is then added to the solid support using procedures described in Example 7. The oligonucleotide synthesis is then completed using procedures described in Example 9 in order to form oligonucleotide 242.

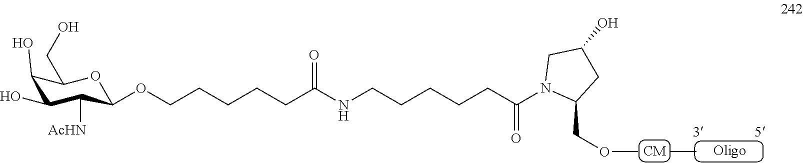

The GalNAc₁ cluster portion (GalNAc₁-29ₐ) of the conjugate group GalNAc₁-29 can be combined with any cleavable moiety present on the oligonucleotide to provide a variety of conjugate groups. The structure of GalNAc₁-29 (GalNAc₁-29ₐ-CM) is shown below:

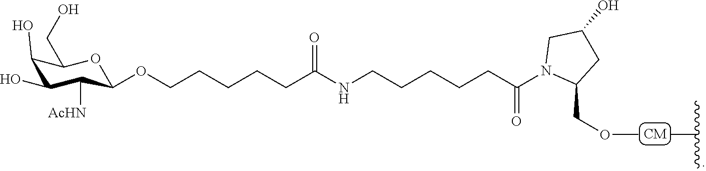

### Example 110: Synthesis of Oligonucleotides Comprising a GalNAc₁-30 Conjugate

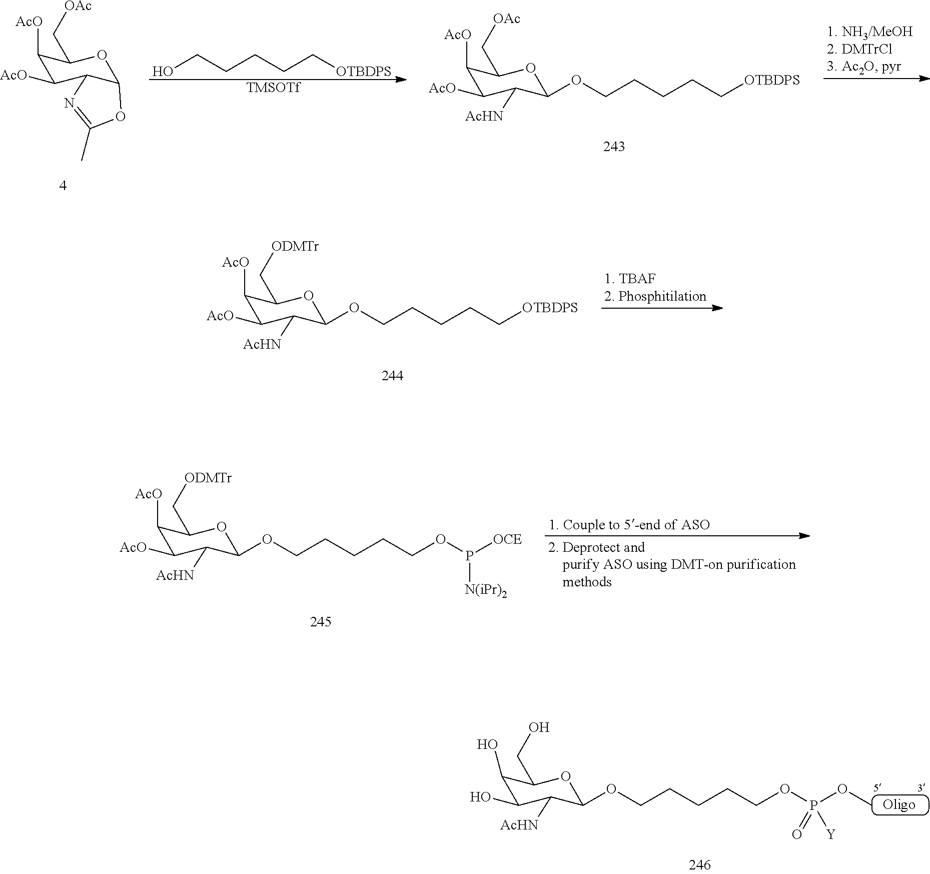

Oligonucleotide 246 comprising a GalNAc₁-30 conjugate group, wherein Y is selected from O, S, a substituted or unsubstituted C₁-C₁₀ alkyl, amino, substituted amino, azido, alkenyl or alkynyl, is synthesized as shown above. The GalNAc₁ cluster portion (GalNAc₁-30ₐ) of the conjugate group GalNAc₁-30 can be combined with any cleavable moiety to provide a variety of conjugate groups. In certain embodiments, Y is part of the cleavable moiety. In certain embodiments, Y is part of a stable moiety, and the cleavable moiety is present on the oligonucleotide. The structure of GalNAc₁-30ₐ is shown below:

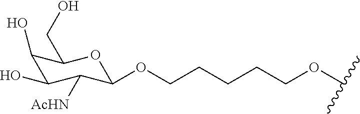 [SMILES: *OCCCCCOC1OC(CO)C(O)C(O)C1NC(C)=O]

### Example 111: Synthesis of Oligonucleotides Comprising a GalNAc₂-31 or GalNAc₂-32 Conjugate

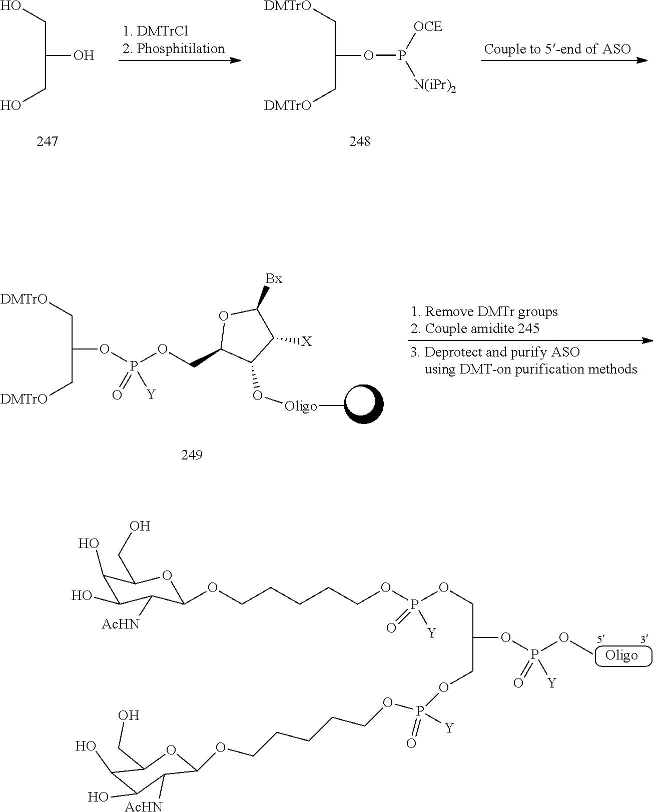

Oligonucleotide 250 comprising a GalNAc₂-31 conjugate group, wherein Y is selected from O, S, a substituted or unsubstituted C₁-C₁₀ alkyl, amino, substituted amino, azido, alkenyl or alkynyl, is synthesized as shown above. The GalNAc2 cluster portion (GalNAc₂-31ₐ) of the conjugate group GalNAc₂-31 can be combined with any cleavable moiety to provide a variety of conjugate groups. In certain embodiments, the Y-containing group directly adjacent to the 5′-end of the oligonucleotide is part of the cleavable moiety. In certain embodiments, the Y-containing group directly adjacent to the 5′-end of the oligonucleotide is part of a stable moiety, and the cleavable moiety is present on the oligonucleotide. The structure of GalNAc₂-31ₐ is shown below:

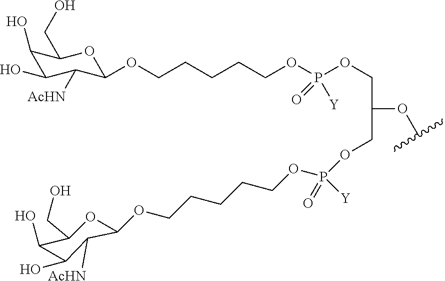

The synthesis of an oligonucleotide comprising a GalNAc₂-32 conjugate is shown below.

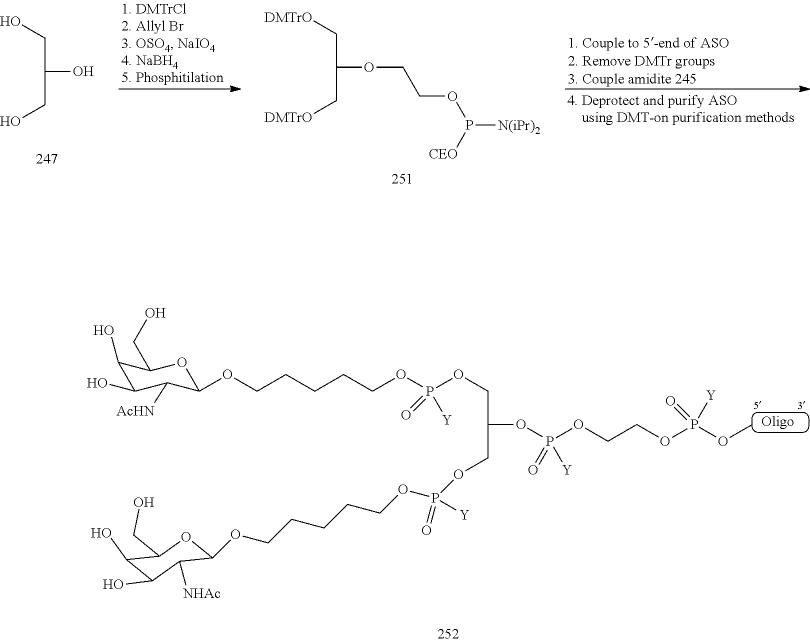

Oligonucleotide 252 comprising a GalNAc₂-32 conjugate group, wherein Y is selected from O, S, a substituted or unsubstituted C₁-C₁₀ alkyl, amino, substituted amino, azido, alkenyl or alkynyl, is synthesized as shown above. The GalNAc2 cluster portion (GalNAc₂-32a) of the conjugate group GalNAc₂-32 can be combined with any cleavable moiety to provide a variety of conjugate groups. In certain embodiments, the Y-containing group directly adjacent to the 5′-end of the oligonucleotide is part of the cleavable moiety. In certain embodiments, the Y-containing group directly adjacent to the 5′-end of the oligonucleotide is part of a stable moiety, and the cleavable moiety is present on the oligonucleotide. The structure of GalNAc₂-32a is shown below:

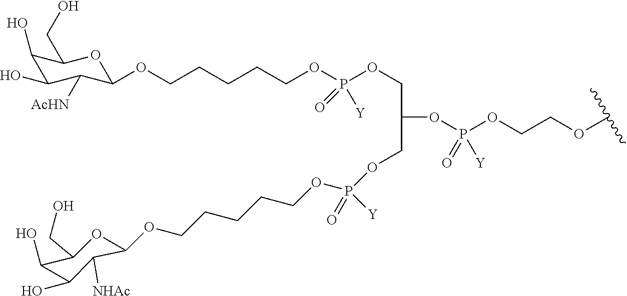

### Example 112: Modified Oligonucleotides Comprising a GalNAc₁ Conjugate

The oligonucleotides in Table 120 targeting SRB-1 were synthesized with a GalNAc₁ conjugate group in order to further test the potency of oligonucleotides comprising conjugate groups that contain one GalNAc ligand.

TABLE 120

| ISIS | | GalNAc | | SEQ |
|---|---|---|---|---|
| No. | Sequences (5′ to 3′) | Cluster | CM | ID No. |
| 711461 | GalNAc₁-25_{a-o′}A_{do}Gₑₛ^{m}CₑₛTₑₛTₑₛ^{m}CₑₛA_{ds}G_{ds}T_{ds} | GalNAc₁-25ₐ | A_{d} | 831 |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| 711462 | GalNAc₁-25_{a-o′}Gₑₛ^{m}CₑₛTₑₛTₑₛ^{m}CₑₛA_{ds}G_{ds}T_{ds}^{m}C_{ds} | GalNAc₁-25ₐ | PO | 829 |
| | A_{ds}T_{ds}G_{ds}A_{ds}^{m}C_{ds}T_{ds}Tₑₛ^{m}Cₑₛ^{m}CₑₛTₑₛTₑ | | | |
| 711463 | GalNAc₁-25_{a-o′}Gₑₛ^{m}CₑₒTₑₒTₑₒ^{m}CₑₒA_{ds}G_{ds}T_{ds}^{m}C_{ds} | GalNAc₁-25ₐ | PO | 829 |
| | A_{ds}T_{ds}G_{ds}A_{ds}^{m}C_{ds}T_{ds}Tₑₒ^{m}Cₑₒ^{m}CₑₛTₑₛTₑ | | | |
| 711465 | GalNAc₁-26_{a-o′}A_{do}Gₑₛ^{m}CₑₛTₑₛTₑₛ^{m}CₑₛA_{ds}G_{ds}T_{ds} | GalNAc₁-26ₐ | A_{d} | 831 |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| 711466 | GalNAc₁-26_{a-o′}Gₑₛ^{m}CₑₛTₑₛTₑₛ^{m}CₑₛA_{ds}G_{ds}T_{ds}^{m}C_{ds} | GalNAc₁-26ₐ | PO | 829 |
| | A_{ds}T_{ds}G_{ds}A_{ds}^{m}C_{ds}T_{ds}Tₑₛ^{m}Cₑₛ^{m}CₑₛTₑₛTₑ | | | |
| 711467 | GalNAc₁-26_{a-o′}Gₑₛ^{m}CₑₒTₑₒTₑₒ^{m}CₑₒA_{ds}G_{ds}T_{ds}^{m}C_{ds} | GalNAc₁-26ₐ | PO | 829 |
| | A_{ds}T_{ds}G_{ds}A_{ds}^{m}C_{ds}T_{ds}Tₑₒ^{m}Cₑₒ^{m}CₑₛTₑₛTₑ | | | |
| 711468 | GalNAc₁-28_{a-o′}A_{do}Gₑₛ^{m}CₑₛTₑₛTₑₛ^{m}CₑₛA_{ds}G_{ds}T_{ds} | GalNAc₁-28ₐ | A_{d} | 831 |
| | ^{m}C_{ds}A_{ds}T_{ds}G_{ds}A_{ds}^{m}C_{ds}T_{ds}Tₑₛ^{m}Cₑₛ^{m}CₑₛTₑₛTₑ | | | |
| 711469 | GalNAc₁-28_{a-o′}Gₑₛ^{m}CₑₛTₑₛTₑₛ^{m}CₑₛA_{ds}G_{ds}T_{ds}^{m}C_{ds} | GalNAc₁-28ₐ | PO | 829 |
| | A_{ds}T_{ds}G_{ds}A_{ds}^{m}C_{ds}T_{ds}Tₑₛ^{m}Cₑₛ^{m}CₑₛTₑₛTₑ | | | |
| 711470 | GalNAc₁-28_{a-o′}Gₑₛ^{m}CₑₒTₑₒTₑₒ^{m}CₑₒA_{ds}G_{ds}T_{ds}^{m}C_{ds} | GalNAc₁-28ₐ | PO | 829 |
| | A_{ds}T_{ds}G_{ds}A_{ds}^{m}C_{ds}T_{ds}Tₑₒ^{m}Cₑₒ^{m}CₑₛTₑₛTₑ | | | |
| 713844 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | GalNAc₁-27ₐ | PO | 829 |
| | ^{m}C_{ds}T_{ds}Tₑₛ^{m}Cₑₛ^{m}CₑₛTₑₛT_{eo′-}GalNAc₁-27ₐ | | | |
| 713845 | Gₑₛ^{m}CₑₒTₑₒTₑₒ^{m}CₑₒA_{ds}G_{ds}T_{ds}^{m}C_{ds}A_{ds}T_{ds}G_{ds}A_{ds} | GalNAc₁-27ₐ | PO | 829 |
| | ^{m}C_{ds}T_{ds}Tₑₒ^{m}Cₑₒ^{m}CₑₛTₑₛT_{eo′-}GalNAc₁-27ₐ | | | |
| 713846 | Gₑₛ^{m}CₑₒTₑₒTₑₒ^{m}CₑₒA_{ds}G_{ds}T_{ds}^{m}C_{ds}A_{ds}T_{ds}G_{ds}A_{ds} | GalNAc₁-27ₐ | A_{d} | 830 |
| | ^{m}C_{ds}T_{ds}Tₑₒ^{m}Cₑₒ^{m}CₑₛTₑₛTₑₒA_{do′-}GalNAc₁-27ₐ | | | |
| 713847 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | GalNAc₁-29ₐ | PO | 829 |
| | ^{m}C_{ds}T_{ds}Tₑₛ^{m}Cₑₛ^{m}CₑₛTₑₛT_{eo′-}GalNAc₁-29ₐ | | | |
| 713848 | Gₑₛ^{m}CₑₒTₑₒTₑₒ^{m}CₑₒA_{ds}G_{ds}T_{ds}^{m}C_{ds}A_{ds}T_{ds}G_{ds}A_{ds} | GalNAc₁-29ₐ | PO | 829 |
| | ^{m}C_{ds}T_{ds}Tₑₒ^{m}Cₑₒ^{m}CₑₛTₑₛT_{eo′-}GalNAc₁-29ₐ | | | |
| 713849 | Gₑₛ^{m}CₑₛTₑₛTₑₛ^{m}CₑₛA_{ds}G_{ds}T_{ds}^{m}C_{ds}A_{ds}T_{ds}G_{ds}A_{ds} | GalNAc₁-29ₐ | A_{d} | 830 |
| | ^{m}C_{ds}T_{ds}Tₑₛ^{m}Cₑₛ^{m}CₑₛTₑₛTₑₒA_{do′-}GalNAc₁-29ₐ | | | |
| 713850 | Gₑₛ^{m}CₑₒTₑₒTₑₒ^{m}CₑₒA_{ds}G_{ds}T_{ds}^{m}C_{ds}A_{ds}T_{ds}G_{ds}A_{ds} | GalNAc₁-29ₐ | A_{d} | 830 |
| | ^{m}C_{ds}T_{ds}Tₑₒ^{m}Cₑₒ^{m}CₑₛTₑₛTₑₒA_{do′-}GalNAc₁-29ₐ | | | |

### Example 113: Antisense Inhibition In Vivo by Oligonucleotides Targeting CFB

The oligonucleotides listed in Table 121 were tested in a dose-dependent study for antisense inhibition of human Complement Factor B (CFB) in mice.

TABLE 121
Modified ASOs targeting CFB

| ISIS | | GalNAc₃ | | SEQ |
|---|---|---|---|---|
| No. | Sequences (5′ to 3′) | Cluster | CM | ID No. |
| 588540 | AₑₛTₑₛ^{m}Cₑₛ^{m}Cₑₛ^{m}CₑₛA_{ds}^{m}C_{ds}G_{ds}^{m}C_{ds}^{m}C_{ds}^{m}C_{ds} | n/a | n/a | 440 |
| | ^{m}C_{ds}T_{ds}G_{ds}T_{ds}^{m}Cₑₛ^{m}CₑₛAₑₛGₑₛ^{m}Ce | | | |
| 687301 | GalNAc₃-3ₐ-_{o′}AₑₛTₑₛ^{m}Cₑₛ^{m}Cₑₛ^{m}CₑₛA_{ds}^{m}C_{ds}G_{ds} | GalNAc₃-3a | PO | 440 |
| | ^{m}C_{ds}^{m}C_{ds}^{m}C_{ds}^{m}C_{ds}T_{ds}G_{ds}T_{ds}^{m}Cₑₛ^{m}CₑₛAₑₛGₑₛ^{m}Cₑ | | | |

The structure of GalNAc₃-3ₐ was shown previously in Example 39.

### Treatment

Transgenic mice that express human CFB (Jackson Laboratory, Bar Harbor, ME) were injected subcutaneously once per week for 3 weeks (a total of 4 doses) with an oligonucleotide listed in Table 122 or with saline. The four treatment groups that received ISIS No. 588540 were given 6, 12, 25, or 50 mg/kg per dose. The four treatment groups that received ISIS No. 687301 were given 0.25, 0.5, 2, or 6 mg/kg per dose. Each treatment group consisted of 4 animals. The mice were sacrificed 2 days following the final administration to determine the liver and kidney human CFB and cyclophilin mRNA levels using real-time PCR according to standard protocols. The CFB mRNA levels were normalized to the cyclophilin levels, and the averages for each treatment group were used to determine the dose that achieved 50% inhibition of the human CFB transcript expression (ED₅₀). The results are the averages of four experiments completed with two different primer probe sets and are shown in Table 122.

TABLE 122
Potencies of oligonucleotides targeting human CFB in vivo

| ISIS | ED₅₀ in liver | ED₅₀ in kidney | GalNAc₃ | |
|---|---|---|---|---|
| No. | (mg/kg) | (mg/kg) | Cluster | CM |
| 588540 | 7.9 | 11.7 | n/a | n/a |
| 687301 | 0.49 | 0.35 | GalNAc₃-3a | PO |

Liver transaminase levels, alanine aminotransferase (ALT) and aspartate aminotransferase (AST), in serum were measured relative to saline injected mice using standard protocols. Total bilirubin, BUN, and body weights were also evaluated. The results show that there were no significant changes in any of the treatment groups relative to the saline treated group (data not shown), indicating that both oligonucleotides were very well tolerated.

### Example 114: Antisense Inhibition In Vivo by Oligonucleotides Targeting CFB

The oligonucleotides listed in Table 123 were tested in a dose-dependent study for antisense inhibition of human CFB in mice.

### Treatment

Transgenic mice that express human CFB (Jackson Laboratory, Bar Harbor, ME) were injected subcutaneously once with 0.6, 1, 6, or 18 mg/kg of an oligonucleotide listed in Table 123 or with saline. Each treatment group consisted of 4 or 5 animals. The mice were sacrificed 72 hours following the dose to determine the liver human CFB and cyclophilin mRNA levels using real-time PCR according to standard protocols. The CFB mRNA levels were normalized to the cyclophilin levels, and the averages for each treatment group were used to determine the dose that achieved 50% inhibition of the human CFB transcript expression (ED₅₀). The results are shown in Table 123.

TABLE 123
Modified ASOs targeting CFB

| | | | | ED₅₀ in | |
|---|---|---|---|---|---|
| ISIS | | GalNAc₃ | | liver | SEQ |
| No. | Sequences (5′ to 3′) | Cluster | CM | (mg/kg) | ID No. |
| 696844 | GalNAc₃-7ₐ-_{o′}AₑₛTₑₛ^{m}Cₑₛ^{m}Cₑₛ^{m}CₑₛA_{ds}^{m}C_{ds}G_{ds} | GalNAc₃-7a | PO | 0.86 | 440 |
| | ^{m}C_{ds}^{m}C_{ds}^{m}C_{ds}^{m}C_{ds}T_{ds}G_{ds}T_{ds}^{m}Cₑₛ^{m}CₑₛAₑₛGₑₛ^{m}Cₑ | | | | |
| 696845 | GalNAc₃-7ₐ-_{o′}AₑₛTₑₒ^{m}Cₑₒ^{m}Cₑₒ^{m}CₑₒA_{ds}^{m}C_{ds}G_{ds} | GalNAc₃-7a | PO | 0.71 | 440 |
| | ^{m}C_{ds}^{m}C_{ds}^{m}C_{ds}^{m}C_{ds}T_{ds}G_{ds}T_{ds}^{m}Cₑₒ^{m}CₑₒAₑₛGₑₛ^{m}Cₑ | | | | |
| 698969 | GalNAc₃-7ₐ-_{o′}AₑₛTₑₒ^{m}Cₑₒ^{m}Cₑₒ^{m}CₑₛA_{ds}^{m}C_{ds}G_{ds} | GalNAc₃-7a | PO | 0.51 | 440 |
| | ^{m}C_{ds}^{m}C_{ds}^{m}C_{ds}^{m}C_{ds}T_{ds}G_{ds}T_{ds}^{m}Cₑₒ^{m}CₑₒAₑₛGₑₛ^{m}Cₑ | | | | |
| 698970 | GalNAc₃-7ₐ-_{o′}AₑₛTₑₛ^{m}Cₑₒ^{m}Cₑₒ^{m}CₑₒA_{ds}^{m}C_{ds}G_{ds} | GalNAc₃-7a | PO | 0.55 | 440 |
| | ^{m}C_{ds}^{m}C_{ds}^{m}C_{ds}^{m}C_{ds}T_{ds}G_{ds}T_{ds}^{m}Cₑₒ^{m}CₑₒAₑₛGₑₛ^{m}Cₑ | | | | |

The structure of GalNAc₃-7ₐ was shown previously in Example 48.

### Example 115: Antisense Inhibition of Human Complement Factor B (CFB) in HepG2 Cells by MOE Gapmers

Antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 4,500 nM antisense oligonucleotide. After a treatment period of approximately 24 hours, RNA was isolated from the cells and CFB mRNA levels were measured by quantitative real-time PCR. Human primer probe set RTS3459 (forward sequence AGTCTCTGTGGCATGGTTTGG, designated herein as SEQ ID NO: 810; reverse sequence GGGCGAATGACTGAGATCTTG, designated herein as SEQ ID NO: 811; probe sequence TACCGATTACCACAAGCAACCATGGCA, designated herein as SEQ ID NO: 812) was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 5-10-5 MOE gapmers. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 124
Inhibition of CFB mRNA by 5-10-5 MOE gapmers targeting SEQ ID NO: 1 and 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | Region | Sequence | bition | site | site | NO: |
| 532608 | 20 | 39 | Exon 1 | GCTGAGCTGCCAGTCAAGGA | 36 | 1741 | 1760 | 6 |
| 532609 | 26 | 45 | Exon 1 | GGCCCCGCTGAGCTGCCAGT | 16 | 1747 | 1766 | 7 |
| 532610 | 45 | 64 | Exon 1 | CGGAACATCCAAGCGGGAGG | 11 | 1766 | 1785 | 8 |
| 532611 | 51 | 70 | Exon 1 | CTTTCCCGGAACATCCAAGC | 26 | 1772 | 1791 | 9 |
| 532612 | 100 | 119 | Exon 1 | ATCTGTGTTCTGGCACCTGC | 25 | 1821 | 1840 | 10 |
| 532613 | 148 | 167 | Exon 1 | GTCACATTCCCTTCCCCTGC | 39 | 1869 | 1888 | 11 |
| 532614 | 154 | 173 | Exon 1 | GACCTGGTCACATTCCCTTC | 71 | 1875 | 1894 | 12 |
| 532615 | 160 | 179 | Exon 1 | GACCTAGACCTGGTCACATT | 35 | 1881 | 1900 | 13 |
| 532616 | 166 | 185 | Exon 1 | ACTCCAGACCTAGACCTGGT | 39 | 1887 | 1906 | 14 |
| 532617 | 172 | 191 | Exon 1 | GCTGAAACTCCAGACCTAGA | 27 | 1893 | 1912 | 15 |
| 532618 | 178 | 197 | Exon 1 | GTCCAAGCTGAAACTCCAGA | 29 | 1899 | 1918 | 16 |
| 532619 | 184 | 203 | Exon 1 | CTCAGTGTCCAAGCTGAAAC | 21 | 1905 | 1924 | 17 |
| 532620 | 246 | 265 | Exon 1 | AGGAGAGAAGCTGGGCCTGG | 31 | 1967 | 1986 | 18 |
| 532621 | 252 | 271 | Exon 1 | GAAGGCAGGAGAGAAGCTGG | 25 | 1973 | 1992 | 19 |
| 532622 | 336 | 355 | Exon 1-2 | GTGGTGGTCACACCTCCAGA | 28 | n/a | n/a | 20 |
| | | | Junction | | | | | |
| 532623 | 381 | 400 | Exon 2 | CCCTCCAGAGAGCAGGATCC | 22 | 2189 | 2208 | 21 |
| 532624 | 387 | 406 | Exon 2 | TCTACCCCCTCCAGAGAGCA | 37 | 2195 | 2214 | 22 |
| 532625 | 393 | 412 | Exon 2 | TTGATCTCTACCCCCTCCAG | 30 | 2201 | 2220 | 23 |
| 532626 | 417 | 436 | Exon 2 | TGGAGAAGTCGGAAGGAGCC | 35 | 2225 | 2244 | 24 |
| 532627 | 423 | 442 | Exon 2 | CCCTCTTGGAGAAGTCGGAA | 37 | 2231 | 2250 | 25 |
| 532628 | 429 | 448 | Exon 2 | GCCTGGCCCTCTTGGAGAAG | 0 | 2237 | 2256 | 26 |
| 532629 | 435 | 454 | Exon 2 | TCCAGTGCCTGGCCCTCTTG | 26 | 2243 | 2262 | 27 |
| 532630 | 458 | 477 | Exon 2 | AGAAGCCAGAAGGACACACG | 30 | 2266 | 2285 | 28 |
| 532631 | 464 | 483 | Exon 2 | ACGGGTAGAAGCCAGAAGGA | 43 | 2272 | 2291 | 29 |
| 532632 | 480 | 499 | Exon 2 | CGTGTCTGCACAGGGTACGG | 57 | 2288 | 2307 | 30 |
| 532633 | 513 | 532 | Exon 2 | AGGGTGCTCCAGGACCCCGT | 27 | 2321 | 2340 | 31 |
| 532634 | 560 | 579 | Exon 2-3 | TTGCTCTGCACTCTGCCTTC | 41 | n/a | n/a | 32 |
| | | | Junction | | | | | |
| 532635 | 600 | 619 | Exon 3 | TATTCCCCGTTCTCGAAGTC | 67 | 2808 | 2827 | 33 |
| 532636 | 626 | 645 | Exon 3 | CATTGTAGTAGGGAGACCGG | 24 | 2834 | 2853 | 34 |
| 532637 | 632 | 651 | Exon 3 | CACTCACATTGTAGTAGGGA | 49 | 2840 | 2859 | 35 |
| 532638 | 638 | 657 | Exon 3 | TCTCATCACTCACATTGTAG | 50 | 2846 | 2865 | 36 |
| 532639 | 644 | 663 | Exon 3 | AAGAGATCTCATCACTCACA | 52 | 2852 | 2871 | 37 |
| 532640 | 650 | 669 | Exon 3 | AGTGGAAAGAGATCTCATCA | 34 | 2858 | 2877 | 38 |
| 532641 | 656 | 675 | Exon 3 | CATAGCAGTGGAAAGAGATC | 32 | 2864 | 2883 | 39 |
| 532642 | 662 | 681 | Exon 3 | AACCGTCATAGCAGTGGAAA | 45 | 2870 | 2889 | 40 |
| 532643 | 668 | 687 | Exon 3 | GAGTGTAACCGTCATAGCAG | 36 | 2876 | 2895 | 41 |
| 532644 | 674 | 693 | Exon 3 | CCCGGAGAGTGTAACCGTCA | 30 | 2882 | 2901 | 42 |
| 532645 | 680 | 699 | Exon 3 | CAGAGCCCCGGAGAGTGTAA | 27 | 2888 | 2907 | 43 |
| 532646 | 686 | 705 | Exon 3 | GATTGGCAGAGCCCCGGAGA | 20 | 2894 | 2913 | 44 |
| 532647 | 692 | 711 | Exon 3 | AGGTGCGATTGGCAGAGCCC | 28 | 2900 | 2919 | 45 |
| 532648 | 698 | 717 | Exon 3 | CTTGGCAGGTGCGATTGGCA | 24 | 2906 | 2925 | 46 |
| 532649 | 704 | 723 | Exon 3 | CATTCACTTGGCAGGTGCGA | 28 | 2912 | 2931 | 47 |
| 532650 | 729 | 748 | Exon 3 | ATCGCTGTCTGCCCACTCCA | 44 | 2937 | 2956 | 48 |
| 532651 | 735 | 754 | Exon 3 | TCACAGATCGCTGTCTGCCC | 44 | 2943 | 2962 | 49 |
| 532652 | 741 | 760 | Exon 3 | CCGTTGTCACAGATCGCTGT | 27 | 2949 | 2968 | 50 |
| 532653 | 747 | 766 | Exon 3-4 | CCCGCTCCGTTGTCACAGAT | 28 | n/a | n/a | 51 |
| | | | Junction | | | | | |
| 532654 | 753 | 772 | Exon 3-4 | CAGTACCCCGCTCCGTTGTC | 13 | n/a | n/a | 52 |
| | | | Junction | | | | | |
| 532655 | 759 | 778 | Exon 3-4 | TTGGAGCAGTACCCCGCTCC | 8 | n/a | n/a | 53 |
| | | | Junction | | | | | |
| 532656 | 789 | 808 | Exon 4 | ACCTTCCTTGTGCCAATGGG | 40 | 3152 | 3171 | 54 |
| 532657 | 795 | 814 | Exon 4 | CTGCCCACCTTCCTTGTGCC | 41 | 3158 | 3177 | 55 |
| 532658 | 818 | 837 | Exon 4 | CGCTGTCTTCAAGGCGGTAC | 33 | 3181 | 3200 | 56 |
| 532659 | 835 | 854 | Exon 4 | GCTGCAGTGGTAGGTGACGC | 32 | 3198 | 3217 | 57 |
| 532660 | 841 | 860 | Exon 4 | CCCCCGGCTGCAGTGGTAGG | 17 | 3204 | 3223 | 58 |
| 532661 | 847 | 866 | Exon 4 | GGTAAGCCCCCGGCTGCAGT | 28 | 3210 | 3229 | 59 |
| 532662 | 853 | 872 | Exon 4 | ACGCAGGGTAAGCCCCCGGC | 13 | 3216 | 3235 | 60 |
| 532663 | 859 | 878 | Exon 4 | GGAGCCACGCAGGGTAAGCC | 33 | 3222 | 3241 | 61 |
| 532664 | 866 | 885 | Exon 4 | GCCGCTGGGAGCCACGCAGG | 10 | 3229 | 3248 | 62 |
| 532665 | 891 | 910 | Exon 4 | CAAGAGCCACCTTCCTGACA | 17 | 3254 | 3273 | 63 |
| 532666 | 897 | 916 | Exon 4 | CCGCTCCAAGAGCCACCTTC | 25 | 3260 | 3279 | 64 |
| 532667 | 903 | 922 | Exon 4 | TCCGTCCCGCTCCAAGAGCC | 29 | 3266 | 3285 | 65 |
| 532668 | 909 | 928 | Exon 4 | GAAGGCTCCGTCCCGCTCCA | 14 | 3272 | 3291 | 66 |
| 532669 | 915 | 934 | Exon 4 | TGGCAGGAAGGCTCCGTCCC | 18 | 3278 | 3297 | 67 |
| 532670 | 921 | 940 | Exon 4-5 | GAGTCTTGGCAGGAAGGCTC | 20 | n/a | n/a | 68 |
| | | | Junction | | | | | |
| 532671 | 927 | 946 | Exon 4-5 | ATGAAGGAGTCTTGGCAGGA | 14 | n/a | n/a | 69 |
| | | | Junction | | | | | |
| 532672 | 956 | 975 | Exon 5 | CTTCGGCCACCTCTTGAGGG | 45 | 3539 | 3558 | 70 |
| 532673 | 962 | 981 | Exon 5 | GGAAAGCTTCGGCCACCTCT | 37 | 3545 | 3564 | 71 |
| 532674 | 968 | 987 | Exon 5 | AAGACAGGAAAGCTTCGGCC | 28 | 3551 | 3570 | 72 |
| 532675 | 974 | 993 | Exon 5 | TCAGGGAAGACAGGAAAGCT | 16 | 3557 | 3576 | 73 |
| 532676 | 996 | 1015 | Exon 5 | TCGACTCCTTCTATGGTCTC | 31 | 3579 | 3598 | 74 |
| 532677 | 1033 | 1052 | Exon 5-6 | CTTCTGTTGTTCCCCTGGGC | 36 | n/a | n/a | 75 |
| | | | Junction | | | | | |
| 532678 | 1068 | 1087 | Exon 6 | TTCATGGAGCCTGAAGGGTC | 19 | 3752 | 3771 | 76 |
| 532679 | 1074 | 1093 | Exon 6 | TAGATGTTCATGGAGCCTGA | 24 | 3758 | 3777 | 77 |
| 532680 | 1080 | 1099 | Exon 6 | ACCAGGTAGATGTTCATGGA | 13 | 3764 | 3783 | 78 |
| 532681 | 1086 | 1105 | Exon 6 | TCTAGCACCAGGTAGATGTT | 20 | 3770 | 3789 | 79 |
| 532682 | 1092 | 1111 | Exon 6 | GATCCATCTAGCACCAGGTA | 33 | 3776 | 3795 | 80 |
| 532683 | 1098 | 1117 | Exon 6 | CTGTCTGATCCATCTAGCAC | 44 | 3782 | 3801 | 81 |
| 532684 | 1104 | 1123 | Exon 6 | CCAATGCTGTCTGATCCATC | 29 | 3788 | 3807 | 82 |
| 532685 | 1129 | 1148 | Exon 6 | TTTGGCTCCTGTGAAGTTGC | 40 | 3813 | 3832 | 83 |

TABLE 125
Inhibition of CFB mRNA by 5-10-5 MOE gapmers targeting SEQ ID NO: 1 and 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | Region | Sequence | bition | site | site | NO: |
| 532686 | 1135 | 1154 | Exon 6 | ACACTTTTTGGCTCCTGTGA | 91 | 3819 | 3838 | 84 |
| 532687 | 1141 | 1160 | Exon 6 | GACTAGACACTTTTTGGCTC | 77 | 3825 | 3844 | 85 |
| 532688 | 1147 | 1166 | Exon 6 | TAAGTTGACTAGACACTTTT | 70 | 3831 | 3850 | 86 |
| 532689 | 1153 | 1172 | Exon 6 | CTCAATTAAGTTGACTAGAC | 61 | 3837 | 3856 | 87 |
| 532690 | 1159 | 1178 | Exon 6-7 | CACCTTCTCAATTAAGTTGA | 57 | 3843 | 3862 | 88 |
| | | | Junction | | | | | |
| 532691 | 1165 | 1184 | Exon 6-7 | ACTTGCCACCTTCTCAATTA | 56 | n/a | n/a | 89 |
| | | | Junction | | | | | |
| 532692 | 1171 | 1190 | Exon 6-7 | ACCATAACTTGCCACCTTCT | 56 | n/a | n/a | 90 |
| | | | Junction | | | | | |
| 532693 | 1177 | 1196 | Exon 7 | CTTCACACCATAACTTGCCA | 56 | 4153 | 4172 | 91 |
| 532694 | 1183 | 1202 | Exon 7 | TCTTGGCTTCACACCATAAC | 55 | 4159 | 4178 | 92 |
| 532695 | 1208 | 1227 | Exon 7 | ATGTGGCATATGTCACTAGA | 55 | 4184 | 4203 | 93 |
| 532696 | 1235 | 1254 | Exon 7 | CAGACACTTTGACCCAAATT | 55 | 4211 | 4230 | 94 |
| 532697 | 1298 | 1317 | Exon 7-8 | GGTCTTCATAATTGATTTCA | 53 | n/a | n/a | 95 |
| | | | Junction | | | | | |
| 532698 | 1304 | 1323 | Exon 7-8 | ACTTGTGGTCTTCATAATTG | 53 | n/a | n/a | 96 |
| | | | Junction | | | | | |
| 532699 | 1310 | 1329 | Exon 7-8 | ACTTCAACTTGTGGTCTTCA | 52 | n/a | n/a | 97 |
| | | | Junction | | | | | |
| 532700 | 1316 | 1335 | Exon 8 | TCCCTGACTTCAACTTGTGG | 52 | 4609 | 4628 | 98 |
| 532701 | 1322 | 1341 | Exon 8 | TGTTAGTCCCTGACTTCAAC | 52 | 4615 | 4634 | 99 |
| 532702 | 1328 | 1347 | Exon 8 | TCTTGGTGTTAGTCCCTGAC | 51 | 4621 | 4640 | 100 |
| 532703 | 1349 | 1368 | Exon 8 | TGTACACTGCCTGGAGGGCC | 51 | 4642 | 4661 | 101 |
| 532704 | 1355 | 1374 | Exon 8 | TCATGCTGTACACTGCCTGG | 51 | 4648 | 4667 | 102 |
| 532705 | 1393 | 1412 | Exon 8 | GTTCCAGCCTTCAGGAGGGA | 50 | 4686 | 4705 | 103 |
| 532706 | 1399 | 1418 | Exon 8 | GGTGCGGTTCCAGCCTTCAG | 50 | 4692 | 4711 | 104 |
| 532707 | 1405 | 1424 | Exon 8 | ATGGCGGGTGCGGTTCCAGC | 50 | 4698 | 4717 | 105 |
| 532708 | 1411 | 1430 | Exon 8 | GATGACATGGCGGGTGCGGT | 49 | 4704 | 4723 | 106 |
| 532709 | 1417 | 1436 | Exon 8 | GAGGATGATGACATGGCGGG | 49 | 4710 | 4729 | 107 |
| 532710 | 1443 | 1462 | Exon 8-9 | CCCATGTTGTGCAATCCATC | 48 | n/a | n/a | 108 |
| | | | Junction | | | | | |
| 532711 | 1449 | 1468 | Exon 9 | TCCCCGCCCATGTTGTGCAA | 48 | 5023 | 5042 | 109 |
| 532712 | 1455 | 1474 | Exon 9 | ATTGGGTCCCCGCCCATGTT | 48 | 5029 | 5048 | 110 |
| 532713 | 1461 | 1480 | Exon 9 | ACAGTAATTGGGTCCCCGCC | 48 | 5035 | 5054 | 111 |
| 532714 | 1467 | 1486 | Exon 9 | TCAATGACAGTAATTGGGTC | 47 | 5041 | 5060 | 112 |
| 532715 | 1473 | 1492 | Exon 9 | ATCTCATCAATGACAGTAAT | 47 | 5047 | 5066 | 113 |
| 532716 | 1479 | 1498 | Exon 9 | TCCCGGATCTCATCAATGAC | 46 | 5053 | 5072 | 114 |
| 532717 | 1533 | 1552 | Exon 9-10 | ACATCCAGATAATCCTCCCT | 46 | n/a | n/a | 115 |
| | | | Junction | | | | | |
| 532718 | 1539 | 1558 | Exon 9-10 | ACATAGACATCCAGATAATC | 46 | n/a | n/a | 116 |
| | | | Junction | | | | | |
| 532719 | 1545 | 1564 | Exon 9-10 | CCAAACACATAGACATCCAG | 46 | n/a | n/a | 117 |
| | | | Junction | | | | | |
| 532720 | 1582 | 1601 | Exon 10 | AGCATTGATGTTCACTTGGT | 46 | 5231 | 5250 | 118 |
| 532721 | 1588 | 1607 | Exon 10 | AGCCAAAGCATTGATGTTCA | 45 | 5237 | 5256 | 119 |
| 532722 | 1594 | 1613 | Exon 10 | CTTGGAAGCCAAAGCATTGA | 45 | 5243 | 5262 | 120 |
| 532723 | 1600 | 1619 | Exon 10 | GTCTTTCTTGGAAGCCAAAG | 45 | 5249 | 5268 | 121 |
| 532724 | 1606 | 1625 | Exon 10 | CTCATTGTCTTTCTTGGAAG | 44 | 5255 | 5274 | 122 |
| 532725 | 1612 | 1631 | Exon 10 | ATGTTGCTCATTGTCTTTCT | 44 | 5261 | 5280 | 123 |
| 532726 | 1618 | 1637 | Exon 10 | GAACACATGTTGCTCATTGT | 44 | 5267 | 5286 | 124 |
| 532727 | 1624 | 1643 | Exon 10 | GACTTTGAACACATGTTGCT | 43 | 5273 | 5292 | 125 |
| 532728 | 1630 | 1649 | Exon 10 | ATCCTTGACTTTGAACACAT | 43 | 5279 | 5298 | 126 |
| 532729 | 1636 | 1655 | Exon 10 | TTCCATATCCTTGACTTTGA | 43 | 5285 | 5304 | 127 |
| 532730 | 1642 | 1661 | Exon 10 | CAGGTTTTCCATATCCTTGA | 42 | 5291 | 5310 | 128 |
| 532731 | 1686 | 1705 | Exon 11 | CTCAGAGACTGGCTTTCATC | 42 | 5827 | 5846 | 129 |
| 532732 | 1692 | 1711 | Exon 11 | CAGAGACTCAGAGACTGGCT | 42 | 5833 | 5852 | 130 |
| 516252 | 1698 | 1717 | Exon 11 | ATGCCACAGAGACTCAGAGA | 42 | 5839 | 5858 | 131 |
| 532733 | 1704 | 1723 | Exon 11 | CAAACCATGCCACAGAGACT | 41 | 5845 | 5864 | 132 |
| 532734 | 1710 | 1729 | Exon 11 | TGTTCCCAAACCATGCCACA | 41 | 5851 | 5870 | 133 |
| 532735 | 1734 | 1753 | Exon 11 | TTGTGGTAATCGGTACCCTT | 41 | 5875 | 5894 | 134 |
| 532736 | 1740 | 1759 | Exon 11 | GGTTGCTTGTGGTAATCGGT | 40 | 5881 | 5900 | 135 |
| 532737 | 1746 | 1765 | Exon 11 | TGCCATGGTTGCTTGTGGTA | 40 | 5887 | 5906 | 136 |
| 532738 | 1752 | 1771 | Exon 11 | TTGGCCTGCCATGGTTGCTT | 40 | 5893 | 5912 | 137 |
| 532739 | 1758 | 1777 | Exon 11 | GAGATCTTGGCCTGCCATGG | 38 | 5899 | 5918 | 138 |
| 532740 | 1803 | 1822 | Exon 12 | ACAGCCCCCATACAGCTCTC | 38 | 6082 | 6101 | 139 |
| 532741 | 1809 | 1828 | Exon 12 | GACACCACAGCCCCCATACA | 38 | 6088 | 6107 | 140 |
| 532742 | 1815 | 1834 | Exon 12 | TACTCAGACACCACAGCCCC | 38 | 6094 | 6113 | 141 |
| 532743 | 1821 | 1840 | Exon 12 | ACAAAGTACTCAGACACCAC | 37 | 6100 | 6119 | 142 |
| 532744 | 1827 | 1846 | Exon 12 | GTCAGCACAAAGTACTCAGA | 37 | 6106 | 6125 | 143 |
| 532745 | 1872 | 1891 | Exon 12 | TTGATTGAGTGTTCCTTGTC | 36 | 6151 | 6170 | 144 |
| 532746 | 1878 | 1897 | Exon 12 | CTGACCTTGATTGAGTGTTC | 35 | 6157 | 6176 | 145 |
| 532747 | 1909 | 1928 | Exon 13 | TATCTCCAGGTCCCGCTTCT | 35 | 6403 | 6422 | 146 |
| 532748 | 1967 | 1986 | Exon 13 | GAATTCCTGCTTCTTTTTTC | 32 | 6461 | 6480 | 147 |
| 532749 | 1973 | 1992 | Exon 13 | ATTCAGGAATTCCTGCTTCT | 32 | 6467 | 6486 | 148 |
| 532750 | 1979 | 1998 | Exon 13 | CATAAAATTCAGGAATTCCT | 32 | 6473 | 6492 | 149 |
| 532751 | 1985 | 2004 | Exon 13 | CATAGTCATAAAATTCAGGA | 31 | 6479 | 6498 | 150 |
| 532752 | 2006 | 2025 | Exon 13 | TGAGCTTGATCAGGGCAACG | 30 | 6500 | 6519 | 151 |
| 532753 | 2012 | 2031 | Exon 13 | TATTCTTGAGCTTGATCAGG | 30 | 6506 | 6525 | 152 |
| 532754 | 2048 | 2067 | Exon 13-14 | GACAAATGGGCCTGATAGTC | 30 | n/a | n/a | 153 |
| | | | Junction | | | | | |
| 532755 | 2070 | 2089 | Exon 14 | GTTGTTCCCTCGGTGCAGGG | 29 | 6659 | 6678 | 154 |
| 532756 | 2076 | 2095 | Exon 14 | GCTCGAGTTGTTCCCTCGGT | 28 | 6665 | 6684 | 155 |
| 532757 | 2082 | 2101 | Exon 14 | CTCAAAGCTCGAGTTGTTCC | 28 | 6671 | 6690 | 156 |
| 532758 | 2088 | 2107 | Exon 14 | GGAAGCCTCAAAGCTCGAGT | 25 | 6677 | 6696 | 157 |
| 532759 | 2094 | 2113 | Exon 14 | GTTGGAGGAAGCCTCAAAGC | 23 | 6683 | 6702 | 158 |
| 532760 | 2100 | 2119 | Exon 14 | GTGGTAGTTGGAGGAAGCCT | 23 | 6689 | 6708 | 159 |
| 532761 | 2106 | 2125 | Exon 14 | TGGCAAGTGGTAGTTGGAGG | 18 | 6695 | 6714 | 160 |
| 532762 | 2112 | 2131 | Exon 14 | TGTTGCTGGCAAGTGGTAGT | 14 | 6701 | 6720 | 161 |

TABLE 126
Inhibition of CFB mRNA by 5-10-5 MOE gapmers targeting SEQ ID NO: 1 and 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | Region | Sequence | bition | site | site | NO: |
| 532812 | n/a | n/a | Exon 1 | TCCAGCTCACTCCCCTGTTG | 19 | 1593 | 1612 | 162 |
| 532813 | n/a | n/a | Exon 1 | TAAGGATCCAGCTCACTCCC | 40 | 1599 | 1618 | 163 |
| 532814 | n/a | n/a | Exon 1 | CAGAAATAAGGATCCAGCTC | 39 | 1605 | 1624 | 164 |
| 532815 | n/a | n/a | Exon 1 | AGGGACCAGAAATAAGGATC | 0 | 1611 | 1630 | 165 |
| 532816 | n/a | n/a | Exon 1 | CCACTTAGGGACCAGAAATA | 27 | 1617 | 1636 | 166 |
| 532817 | n/a | n/a | Exon 1 | TCCAGGACTCTCCCCTTCAG | 39 | 1682 | 1701 | 167 |
| 532818 | n/a | n/a | Exon 1 | AAGTCCCACCCTTTGCTGCC | 15 | 1707 | 1726 | 168 |
| 532819 | n/a | n/a | Exon 1 | CTGCAGAAGTCCCACCCTTT | 26 | 1713 | 1732 | 169 |
| 532820 | n/a | n/a | Exon 1 | CAGAAACTGCAGAAGTCCCA | 8 | 1719 | 1738 | 170 |
| 532821 | n/a | n/a | Exon 2- | AACCTCTGCACTCTGCCTTC | 39 | 2368 | 2387 | 171 |
| | | | Intron | | | | | |
| | | | 2 | | | | | |
| 532822 | n/a | n/a | Exon 2- | CCCTCAAACCTCTGCACTCT | 3 | 2374 | 2393 | 172 |
| | | | Intron | | | | | |
| | | | 2 | | | | | |
| 532823 | n/a | n/a | Exon 2- | TCATTGCCCTCAAACCTCTG | 19 | 2380 | 2399 | 173 |
| | | | Intron | | | | | |
| | | | 2 | | | | | |
| 532824 | n/a | n/a | Intron 2 | CCACACTCATTGCCCTCAAA | 37 | 2386 | 2405 | 174 |
| 532825 | n/a | n/a | Intron 2 | CACTGCCCACACTCATTGCC | 23 | 2392 | 2411 | 175 |
| 532826 | n/a | n/a | Intron 2 | TTAGGCCACTGCCCACACTC | 15 | 2398 | 2417 | 176 |
| 532827 | n/a | n/a | Intron 2 | CTAGTCCTGACCTTGCTGCC | 28 | 2436 | 2455 | 177 |
| 532828 | n/a | n/a | Intron 2 | CTCATCCTAGTCCTGACCTT | 25 | 2442 | 2461 | 178 |
| 532829 | n/a | n/a | Intron 2 | CCTAGTCTCATCCTAGTCCT | 23 | 2448 | 2467 | 179 |
| 532830 | n/a | n/a | Intron 2 | ACCCTGCCTAGTCTCATCCT | 30 | 2454 | 2473 | 180 |
| 532831 | n/a | n/a | Intron 2 | CTTGTCACCCTGCCTAGTCT | 34 | 2460 | 2479 | 181 |
| 532832 | n/a | n/a | Intron 2 | GCCCACCTTGTCACCCTGCC | 36 | 2466 | 2485 | 182 |
| 532833 | n/a | n/a | Intron 2 | CCTAAAACTGCTCCTACTCC | 9 | 2492 | 2511 | 183 |
| 532834 | n/a | n/a | Intron 4 | GAGTCAGAAATGAGGTCAAA | 19 | 3494 | 3513 | 184 |
| 532835 | n/a | n/a | Intron | CCCTACTCCCATTTCACCTT | 16 | 5971 | 5990 | 185 |
| | | | 11 | | | | | |
| 532836 | n/a | n/a | Intron 8- | TGTTGTGCAATCCTGCAGAA | 25 | 5013 | 5032 | 186 |
| | | | Exon | | | | | |
| | | | 9 | | | | | |
| 532837 | n/a | n/a | Intron 1 | AAAGGCTGATGAAGCCTGGC | 18 | 2123 | 2142 | 187 |
| 532838 | n/a | n/a | Intron 7 | CCTTTGACCACAAAGTGGCC | 21 | 4461 | 4480 | 188 |
| 532839 | n/a | n/a | Intron | AGGTACCACCTCTTTGTGGG | 29 | 6362 | 6381 | 189 |
| | | | 12 | | | | | |
| 532840 | n/a | n/a | Intron 1- | TGGTGGTCACACCTGAAGAG | 34 | 2143 | 2162 | 190 |
| | | | Exon | | | | | |
| | | | 2 | | | | | |
| 532763 | 2133 | 2152 | Exon | GCAGGGAGCAGCTCTTCCTT | 40 | n/a | n/a | 191 |
| | | | 14-15 | | | | | |
| | | | Junction | | | | | |
| 532764 | 2139 | 2158 | Exon 15 | TCCTGTGCAGGGAGCAGCTC | 28 | 6927 | 6946 | 192 |
| 532765 | 2145 | 2164 | Exon 15 | TTGATATCCTGTGCAGGGAG | 41 | 6933 | 6952 | 193 |
| 532766 | 2151 | 2170 | Exon 15 | AGAGCTTTGATATCCTGTGC | 36 | 6939 | 6958 | 194 |
| 532767 | 2157 | 2176 | Exon 15 | ACAAACAGAGCTTTGATATC | 33 | 6945 | 6964 | 195 |
| 532768 | 2163 | 2182 | Exon 15 | TCAGACACAAACAGAGCTTT | 41 | 6951 | 6970 | 196 |
| 532769 | 2169 | 2188 | Exon 15 | TCCTCCTCAGACACAAACAG | 49 | 6957 | 6976 | 197 |
| 532770 | 2193 | 2212 | Exon 15 | ACCTCCTTCCGAGTCAGCTT | 61 | 6981 | 7000 | 198 |
| 532771 | 2199 | 2218 | Exon 15 | ATGTAGACCTCCTTCCGAGT | 39 | 6987 | 7006 | 199 |
| 532772 | 2205 | 2224 | Exon 15 | TTCTTGATGTAGACCTCCTT | 30 | 6993 | 7012 | 200 |
| 532773 | 2211 | 2230 | Exon 15 | TCCCCATTCTTGATGTAGAC | 31 | 6999 | 7018 | 201 |
| 532774 | 2217 | 2236 | Exon | TTCTTATCCCCATTCTTGAT | 36 | n/a | n/a | 202 |
| | | | 15-16 | | | | | |
| | | | Junction | | | | | |
| 532775 | 2223 | 2242 | Exon | CTGCCTTTCTTATCCCCATT | 56 | n/a | n/a | 203 |
| | | | 15-16 | | | | | |
| | | | Junction | | | | | |
| 532776 | 2229 | 2248 | Exon | TCACAGCTGCCTTTCTTATC | 33 | n/a | n/a | 204 |
| | | | 15-16 | | | | | |
| | | | Junction | | | | | |
| 532777 | 2235 | 2254 | Exon 16 | TCTCTCTCACAGCTGCCTTT | 38 | 7119 | 7138 | 205 |
| 532778 | 2241 | 2260 | Exon 16 | TGAGCATCTCTCTCACAGCT | 51 | 7125 | 7144 | 206 |
| 532779 | 2247 | 2266 | Exon 16 | GCATATTGAGCATCTCTCTC | 39 | 7131 | 7150 | 207 |
| 532780 | 2267 | 2286 | Exon 16 | TGACTTTGTCATAGCCTGGG | 56 | 7151 | 7170 | 208 |
| 532781 | 2273 | 2292 | Exon 16 | TGTCCTTGACTTTGTCATAG | 36 | 7157 | 7176 | 209 |
| 532782 | 2309 | 2328 | Exon 16 | CAGTACAAAGGAACCGAGGG | 30 | 7193 | 7212 | 210 |
| 532783 | 2315 | 2334 | Exon 16 | CTCCTCCAGTACAAAGGAAC | 21 | 7199 | 7218 | 211 |
| 532784 | 2321 | 2340 | Exon 16 | GACTCACTCCTCCAGTACAA | 31 | 7205 | 7224 | 212 |
| 532785 | 2327 | 2346 | Exon 16 | CATAGGGACTCACTCCTCCA | 30 | 7211 | 7230 | 213 |
| 532786 | 2333 | 2352 | Exon 16 | GGTCAGCATAGGGACTCACT | 31 | 7217 | 7236 | 214 |
| 532787 | 2352 | 2371 | Exon | TCACCTCTGCAAGTATTGGG | 42 | 7236 | 7255 | 215 |
| | | | 16-17 | | | | | |
| | | | Junction | | | | | |
| 532788 | 2358 | 2377 | Exon | CCAGAATCACCTCTGCAAGT | 32 | n/a | n/a | 216 |
| | | | 16-17 | | | | | |
| | | | Junction | | | | | |
| 532789 | 2364 | 2383 | Exon | GGGCCGCCAGAATCACCTCT | 35 | n/a | n/a | 217 |
| | | | 16-17 | | | | | |
| | | | Junction | | | | | |
| 532790 | 2382 | 2401 | Exon 17 | CTCTTGTGAACTATCAAGGG | 33 | 7347 | 7366 | 218 |
| 532791 | 2388 | 2407 | Exon 17 | CGACTTCTCTTGTGAACTAT | 52 | 7353 | 7372 | 219 |
| 532792 | 2394 | 2413 | Exon 17 | ATGAAACGACTTCTCTTGTG | 16 | 7359 | 7378 | 220 |
| 532793 | 2400 | 2419 | Exon | ACTTGAATGAAACGACTTCT | 45 | 7365 | 7384 | 221 |
| | | | 17-18 | | | | | |
| | | | Junction | | | | | |
| 532794 | 2406 | 2425 | Exon | ACACCAACTTGAATGAAACG | 18 | n/a | n/a | 222 |
| | | | 17-18 | | | | | |
| | | | Junction | | | | | |
| 532795 | 2427 | 2446 | Exon 18 | TCCACTACTCCCCAGCTGAT | 30 | 7662 | 7681 | 223 |
| 532796 | 2433 | 2452 | Exon 18 | CAGACATCCACTACTCCCCA | 38 | 7668 | 7687 | 224 |
| 532797 | 2439 | 2458 | Exon 18 | TTTTTGCAGACATCCACTAC | 35 | 7674 | 7693 | 225 |
| 532798 | 2445 | 2464 | Exon 18 | TTCTGGTTTTTGCAGACATC | 45 | 7680 | 7699 | 226 |
| 532799 | 2451 | 2470 | Exon 18 | TGCCGCTTCTGGTTTTTGCA | 47 | 7686 | 7705 | 227 |
| 532800 | 2457 | 2476 | Exon 18 | TGCTTTTGCCGCTTCTGGTT | 61 | 7692 | 7711 | 228 |
| 532801 | 2463 | 2482 | Exon 18 | GGTACCTGCTTTTGCCGCTT | 47 | 7698 | 7717 | 229 |
| 532802 | 2469 | 2488 | Exon 18 | TGAGCAGGTACCTGCTTTTG | 31 | 7704 | 7723 | 230 |
| 532803 | 2517 | 2536 | Exon 18 | TTCAGCCAGGGCAGCACTTG | 41 | 7752 | 7771 | 231 |
| 532804 | 2523 | 2542 | Exon 18 | TTCTCCTTCAGCCAGGGCAG | 44 | 7758 | 7777 | 232 |
| 532805 | 2529 | 2548 | Exon 18 | TGGAGTTTCTCCTTCAGCCA | 46 | 7764 | 7783 | 233 |
| 532806 | 2535 | 2554 | Exon 18 | TCATCTTGGAGTTTCTCCTT | 49 | 7770 | 7789 | 234 |
| 532807 | 2541 | 2560 | Exon 18 | AAATCCTCATCTTGGAGTTT | 30 | 7776 | 7795 | 235 |
| 532808 | 2547 | 2566 | Exon 18 | AAACCCAAATCCTCATCTTG | 20 | 7782 | 7801 | 236 |
| 532809 | 2571 | 2590 | Exon 18 | GTCCAGCAGGAAACCCCTTA | 65 | 7806 | 7825 | 237 |
| 532810 | 2577 | 2596 | Exon 18 | GCCCCTGTCCAGCAGGAAAC | 74 | 7812 | 7831 | 238 |
| 532811 | 2599 | 2618 | Exon 18 | AGCTGTTTTAATTCAATCCC | 96 | 7834 | 7853 | 239 |

TABLE 127
Inhibition of CFB mRNA by 5-10-5 MOE gapmers targeting SEQ ID NO: 1 and 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | Region | Sequence | bition | site | site | NO: |
| 532841 | n/a | n/a | Intron 6- | AACTTGCCACCTGTGGGTGA | 4142 | 4161 | 11 | 240 |
| | | | Exon 7 | | | | | |
| 532842 | n/a | n/a | Exon 15- | TCACCTTATCCCCATTCTTG | 7007 | 7026 | 16 | 241 |
| | | | Intron 15 | | | | | |
| 532843 | n/a | n/a | Intron 11 | TCAACTTTCACAAACCACCA | 6015 | 6034 | 19 | 242 |
| 532844 | n/a | n/a | Intron 16- | CCGCCAGAATCACCTGCAAG | 7326 | 7345 | 33 | 243 |
| | | | Exon 17 | | | | | |
| 532845 | n/a | n/a | Intron 10 | AGGAGGAATGAAGAAGGCTT | 5431 | 5450 | 29 | 244 |
| 532846 | n/a | n/a | Intron 13 | GCCTTTCCTCAGGGATCTGG | 6561 | 6580 | 26 | 245 |
| 532847 | n/a | n/a | Intron 4 | AAATGTCTGGGAGTGTCAGG | 3477 | 3496 | 18 | 246 |
| 532848 | n/a | n/a | Intron 15 | GCCTAGAGTGCCTCCTTAGG | 7038 | 7057 | 20 | 247 |
| 532849 | n/a | n/a | Intron 17 | GGCATCTCCCCAGATAGGAA | 7396 | 7415 | 16 | 248 |
| 532850 | n/a | n/a | Intron 6 | AGGGAGCTAGTCCTGGAAGA | 3906 | 3925 | 14 | 249 |
| 532851 | n/a | n/a | Intron 1- | ACACCTGAAGAGAAAGGCTG | 2135 | 2154 | 6 | 250 |
| | | | Exon 2 | | | | | |
| 532852 | n/a | n/a | Intron 7 | CCCTTTGACCACAAAGTGGC | 4462 | 4481 | 25 | 251 |
| 532853 | n/a | n/a | Intron 7 | GCCCTCAAGGTAGTCTCATG | 4354 | 4373 | 26 | 252 |
| 532854 | n/a | n/a | Intron 6 | AAGGGAAGGAGGACAGAATA | 3977 | 3996 | 18 | 253 |
| 532855 | n/a | n/a | Intron 1 | AAAGGCCAAGGAGGGATGCT | 2099 | 2118 | 9 | 254 |
| 532856 | n/a | n/a | Exon 8- | AGAGGTCCCTTCTGACCATC | 4736 | 4755 | 4 | 255 |
| | | | Intron 8 | | | | | |
| 532857 | n/a | n/a | Intron 8 | GCTGGGACAGGAGAGAGGTC | 4749 | 4768 | 0 | 256 |
| 532858 | n/a | n/a | Intron 4 | TCAAATGTCTGGGAGTGTCA | 3479 | 3498 | 13 | 257 |
| 532859 | n/a | n/a | Intron 10 | AGAAGGAGAATGTGCTGAAA | 5801 | 5820 | 20 | 258 |
| 532860 | n/a | n/a | Intron 17 | TGCTGACCACTTGGCATCTC | 7408 | 7427 | 20 | 259 |
| 532861 | n/a | n/a | Intron 11 | CAACTTTCACAAACCACCAT | 6014 | 6033 | 18 | 260 |
| 532862 | n/a | n/a | Intron 10 | AGCTCTGTGATTCTAAGGTT | 5497 | 5516 | 15 | 261 |
| 532863 | n/a | n/a | Intron 6- | CCACCTGTGGGTGAGGAGAA | 4136 | 4155 | 16 | 262 |
| | | | Exon 7 | | | | | |
| 532864 | n/a | n/a | Exon 17- | GAGGACTCACTTGAATGAAA | 7373 | 7392 | 21 | 263 |
| | | | Intron 17 | | | | | |
| 532865 | n/a | n/a | Intron 6 | TGGAATGATCAGGGAGCTAG | 3916 | 3935 | 30 | 264 |
| 532866 | n/a | n/a | Intron 5 | GTCCCTTCTCCATTTTCCCC | 3659 | 3678 | 26 | 265 |
| 532867 | n/a | n/a | Intron 7 | TCAACTTTTTAAGTTAATCA | 4497 | 4516 | 14 | 266 |
| 532868 | n/a | n/a | Intron 6 | GGGTGAGGAGAACAAGGCGC | 4128 | 4147 | 21 | 267 |
| 532869 | n/a | n/a | Intron 7 | CTTCCAAGCCATCTTTTAAC | 4553 | 4572 | 5 | 268 |
| 532870 | n/a | n/a | Exon 17- | AGGACTCACTTGAATGAAAC | 7372 | 7391 | 18 | 269 |
| | | | Intron 17 | | | | | |
| 532871 | n/a | n/a | Intron 10 | TTCCAGGCAACTAGAGCTTC | 5412 | 5431 | 15 | 270 |
| 532872 | n/a | n/a | Exon 1 | CAGAGTCCAGCCACTGTTTG | 1557 | 1576 | 13 | 271 |
| 532873 | n/a | n/a | Intron 17- | CCAACCTGCAGAGGCAGTGG | 7638 | 7657 | 23 | 272 |
| | | | Exon 18 | | | | | |
| 532874 | n/a | n/a | Intron 16 | TGCAAGGAGAGGAGAAGCTG | 7312 | 7331 | 10 | 273 |
| 532875 | n/a | n/a | Exon 9- | CTAGGCAGGTTACTCACCCA | 5120 | 5139 | 21 | 274 |
| | | | Intron 9 | | | | | |
| 532876 | n/a | n/a | Intron 6- | CACCATAACTTGCCACCTGT | 4148 | 4167 | 41 | 275 |
| | | | Exon 7 | | | | | |
| 532877 | n/a | n/a | Intron 12 | TAGGTACCACCTCTTTGTGG | 6363 | 6382 | 27 | 276 |
| 532878 | n/a | n/a | Intron 11 | CTTGACCTCACCTCCCCCAA | 5954 | 5973 | 13 | 277 |
| 532879 | n/a | n/a | Intron 12 | CCACCTCTTTGTGGGCAGCT | 6357 | 6376 | 33 | 278 |
| 532880 | n/a | n/a | Intron 11 | TTCACAAACCACCATCTCTT | 6009 | 6028 | 8 | 279 |
| 532881 | n/a | n/a | Exon 3- | TTCTCACCTCCGTTGTCACA | 2958 | 2977 | 17 | 280 |
| | | | Intron 3 | | | | | |
| 532882 | n/a | n/a | Intron 12 | GAAAGTGGGAGGTGTTGCCT | 6225 | 6244 | 19 | 281 |
| 532883 | n/a | n/a | Intron 1 | ACAGCAGGAAGGGAAGGTTA | 2075 | 2094 | 34 | 282 |
| 532884 | n/a | n/a | Intron 17 | CATGCTGACCACTTGGCATC | 7410 | 7429 | 18 | 283 |
| 532885 | n/a | n/a | Exon 4- | GGTCACCTTGGCAGGAAGGC | 3286 | 3305 | 0 | 284 |
| | | | Intron 4 | | | | | |
| 532886 | n/a | n/a | Intron 8 | GTATAGTGTTACAAGTGGAC | 4804 | 4823 | 13 | 285 |
| 532887 | n/a | n/a | Intron 7 | GGACTTCCCTTTGACCACAA | 4468 | 4487 | 18 | 286 |
| 532888 | n/a | n/a | Intron 11 | TCACCTTGACCTCACCTCCC | 5958 | 5977 | 20 | 287 |
| 532889 | n/a | n/a | Intron 15 | TAGAGTGCCTCCTTAGGATG | 7035 | 7054 | 27 | 288 |
| 532890 | n/a | n/a | Intron 7 | TGACTTCAACTTGTGGTCTG | 4605 | 4624 | 16 | 289 |
| 532891 | n/a | n/a | Intron 10 | CAGAGAAGGAGAATGTGCTG | 5804 | 5823 | 25 | 290 |
| 532892 | n/a | n/a | Intron 14- | AGGGAGCAGCTCTTCCTCTG | 6919 | 6938 | 47 | 291 |
| | | | Exon 15 | | | | | |
| 532893 | n/a | n/a | Intron 5- | TGTTCCCCTGGGTGCCAGGA | 3710 | 3729 | 24 | 292 |
| | | | Exon 6 | | | | | |
| 532894 | n/a | n/a | Intron 10 | GGCCTGGCTGTTTTCAAGCC | 5612 | 5631 | 15 | 293 |
| 532895 | n/a | n/a | Intron 10- | GACTGGCTTTCATCTGGCAG | 5821 | 5840 | 25 | 294 |
| | | | Exon 11 | | | | | |
| 532896 | n/a | n/a | Intron 10 | GAAGGCTTTCCAGGCAACTA | 5419 | 5438 | 19 | 295 |
| 532897 | n/a | n/a | Exon 17- | TCACTTGAATGAAACGACTT | 7367 | 7386 | 11 | 296 |
| | | | Intron 17 | | | | | |
| 532898 | n/a | n/a | Intron 1 | GGCCCCAAAAGGCCAAGGAG | 2106 | 2125 | 5 | 297 |
| 532899 | n/a | n/a | Intron 16- | AATCACCTGCAAGGAGAGGA | 7319 | 7338 | 19 | 298 |
| | | | Exon 17 | | | | | |
| 532900 | n/a | n/a | Intron 12 | GACCTTCAGTTGCATCCTTA | 6183 | 6202 | 25 | 299 |
| 532901 | n/a | n/a | Intron 1 | TGATGAAGCCTGGCCCCAAA | 2117 | 2136 | 0 | 300 |
| 532902 | n/a | n/a | Intron 12 | TAGAAAGTGGGAGGTGTTGC | 6227 | 6246 | 0 | 301 |
| 532903 | n/a | n/a | Intron 12 | CCCATCCCTGACTGGTCTGG | 6295 | 6314 | 14 | 302 |
| 532904 | n/a | n/a | Intron 8 | CCATGGGTATAGTGTTACAA | 4810 | 4829 | 13 | 303 |
| 532905 | n/a | n/a | Intron 2 | GTGTTCTCTTGACTTCCAGG | 2586 | 2605 | 23 | 304 |
| 532906 | n/a | n/a | Intron 13 | GGCCTGCTCCTCACCCCAGT | 6597 | 6616 | 27 | 305 |
| 532907 | n/a | n/a | Intron 10 | GAGGCCTGGCTGTTTTCAAG | 5614 | 5633 | 32 | 306 |
| 532908 | n/a | n/a | Exon 1 | GACTCTCCCCTTCAGTACCT | 1677 | 1696 | 16 | 307 |
| 532909 | n/a | n/a | Intron 8 | CATGGGTATAGTGTTACAAG | 4809 | 4828 | 10 | 308 |
| 532910 | n/a | n/a | Intron 10 | GAAGGAGAATGTGCTGAAAA | 5800 | 5819 | 0 | 309 |
| 532911 | n/a | n/a | Intron 7 | TCACCTGGTCTTCCAAGCCA | 4562 | 4581 | 0 | 310 |
| 532912 | n/a | n/a | Intron 17 | CTCCCCAGATAGGAAAGGGA | 7391 | 7410 | 0 | 311 |
| 532913 | n/a | n/a | Exon 17- | GGACTCACTTGAATGAAACG | 7371 | 7390 | 0 | 312 |
| | | | Intron 17 | | | | | |
| 532914 | n/a | n/a | Intron 16- | GGCCGCCAGAATCACCTGCA | 7328 | 7347 | 30 | 313 |
| | | | Exon 17 | | | | | |
| 532915 | n/a | n/a | Exon 17- | CTCACTTGAATGAAACGACT | 7368 | 7387 | 22 | 314 |
| | | | Intron 17 | | | | | |
| 532916 | n/a | n/a | Intron 13 | CTTTCCCAGCCTTTCCTCAG | 6569 | 6588 | 28 | 315 |
| 532918 | n/a | n/a | Intron 12 | AGAAAGTGGGAGGTGTTGCC | 6226 | 6245 | 3 | 316 |
| 532917 | 2604 | 2623 | Exon 18 | GTCGCAGCTGTTTTAATTCA | 7839 | 7858 | 90 | 317 |

TABLE 128
Inhibition of CFB mRNA by 5-10-5 MOE gapmers targeting SEQ ID NO: 1 and 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | Region | Sequence | bition | site | site | NO: |
| 532919 | n/a | n/a | Exon 1 | CCAGGACTCTCCCCTTCAGT | 1681 | 1700 | 4 | 318 |
| 532920 | n/a | n/a | Intron 6 | AGGGAAGGAGGACAGAATAG | 3976 | 3995 | 25 | 319 |
| 532921 | n/a | n/a | Intron 4 | GAAATGAGGTCAAATGTCTG | 3488 | 3507 | 30 | 320 |
| 532922 | n/a | n/a | Intron 4 | GGAGAGTCAGAAATGAGGTC | 3497 | 3516 | 25 | 321 |
| 532923 | n/a | n/a | Intron 12 | GTAGAAAGTGGGAGGTGTTG | 6228 | 6247 | 26 | 322 |
| 532924 | n/a | n/a | Intron 10 | TAGAAAGATCTCTGAAGTGC | 5521 | 5540 | 24 | 323 |
| 532925 | n/a | n/a | Intron 13 | CTGCTCCTCACCCCAGTCCT | 6594 | 6613 | 26 | 324 |
| 532926 | n/a | n/a | Intron 11 | CTACTGGGATTCTGTGCTTA | 5927 | 5946 | 30 | 325 |
| 532927 | n/a | n/a | Intron 1 | CCCAAAAGGCCAAGGAGGGA | 2103 | 2122 | 13 | 326 |
| 532928 | n/a | n/a | Intron 17 | TGACCACTTGGCATCTCCCC | 7405 | 7424 | 27 | 327 |
| 532929 | n/a | n/a | Intron 16- | CCTGCAAGGAGAGGAGAAGC | 7314 | 7333 | 29 | 328 |
| | | | Exon 17 | | | | | |
| 532930 | n/a | n/a | Exon 16- | CTCTCACCTCTGCAAGTATT | 7239 | 7258 | 44 | 329 |
| | | | Intron 16 | | | | | |
| 532931 | n/a | n/a | Intron 1 | CCCCAAAAGGCCAAGGAGGG | 2104 | 2123 | 21 | 330 |
| 532932 | n/a | n/a | Intron 7 | GTCTTCCAAGCCATCTTTTA | 4555 | 4574 | 20 | 331 |
| 532933 | n/a | n/a | Intron 8 | GTTACAAGTGGACTTAAGGG | 4797 | 4816 | 30 | 332 |
| 532934 | n/a | n/a | Intron 8- | CCCATGTTGTGCAATCCTGC | 5017 | 5036 | 30 | 333 |
| | | | Exon 9 | | | | | |
| 532935 | n/a | n/a | Intron 15 | GAGGTGGGAAGCATGGAGAA | 7091 | 7110 | 17 | 334 |
| 532936 | n/a | n/a | Intron 14 | TGCTCCCACCACTGTCATCT | 6874 | 6893 | 21 | 335 |
| 532937 | n/a | n/a | Exon 9- | AGGCAGGTTACTCACCCAGA | 5118 | 5137 | 18 | 336 |
| | | | Intron 9 | | | | | |
| 532938 | n/a | n/a | Intron 11 | TACTGGGATTCTGTGCTTAC | 5926 | 5945 | 15 | 337 |
| 532939 | n/a | n/a | Intron 13 | GCCTTTCCCAGCCTTTCCTC | 6571 | 6590 | 27 | 338 |
| 532940 | n/a | n/a | Intron 8- | GTGCAATCCTGCAGAAGAGA | 5009 | 5028 | 21 | 339 |
| | | | Exon 9 | | | | | |
| 532941 | n/a | n/a | Intron 8 | ACAGGAGAGAGGTCCCTTCT | 4743 | 4762 | 20 | 340 |
| 532942 | n/a | n/a | Intron 10 | CCCAAAAGGAGAAAGGGAAA | 5717 | 5736 | 14 | 341 |
| 532943 | n/a | n/a | Intron 2 | AAGCCCAGGGTAAATGCTTA | 2557 | 2576 | 32 | 342 |
| 532944 | n/a | n/a | Intron 1 | GATGAAGCCTGGCCCCAAAA | 2116 | 2135 | 22 | 343 |
| 532945 | n/a | n/a | Intron 10 | TGGCAGAGAAGGAGAATGTG | 5807 | 5826 | 22 | 344 |
| 532946 | n/a | n/a | Intron 13 | TTCCCAGCCTTTCCTCAGGG | 6567 | 6586 | 35 | 345 |
| 532947 | n/a | n/a | Intron 10 | GGCAGAGAAGGAGAATGTGC | 5806 | 5825 | 30 | 346 |
| 532948 | n/a | n/a | Intron 10 | ACAGTGCCAGGAAACAAGAA | 5471 | 5490 | 25 | 347 |
| 532949 | n/a | n/a | Exon 9- | TAGGCAGGTTACTCACCCAG | 5119 | 5138 | 22 | 348 |
| | | | Intron 9 | | | | | |
| 532950 | n/a | n/a | Intron 2 | TTCTCTTGACTTCCAGGGCT | 2583 | 2602 | 22 | 349 |
| 532951 | n/a | n/a | Intron 13 | CCTGCTCCTCACCCCAGTCC | 6595 | 6614 | 16 | 350 |
| 532953 | n/a | n/a | Intron 7 | TCCCACTAACCTCCATTGCC | 4422 | 4441 | 14 | 351 |
| 532954 | n/a | n/a | Intron 7 | TTCCCTTTGACCACAAAGTG | 4464 | 4483 | 16 | 352 |
| 532955 | n/a | n/a | Intron 9 | CTGGGTCCTAGGCAGGTTAC | 5127 | 5146 | 30 | 353 |
| 532956 | n/a | n/a | Intron 10 | TCCAGGCAACTAGAGCTTCA | 5411 | 5430 | 20 | 354 |
| 532957 | n/a | n/a | Intron 8- | GCCCATGTTGTGCAATCCTG | 5018 | 5037 | 45 | 355 |
| | | | Exon 9 | | | | | |
| 532958 | n/a | n/a | Intron 7 | GGTTCCCACTAACCTCCATT | 4425 | 4444 | 18 | 356 |
| 532959 | n/a | n/a | Intron 3 | AGGTAGAGAGCAAGAGTTAC | 3052 | 3071 | 26 | 357 |
| 532960 | n/a | n/a | Intron 7 | CCACTAACCTCCATTGCCCA | 4420 | 4439 | 10 | 358 |
| 532961 | n/a | n/a | Intron 11 | TCACAAACCACCATCTCTTA | 6008 | 6027 | 40 | 359 |
| 532962 | n/a | n/a | Exon 9- | TACTCACCCAGATAATCCTC | 5110 | 5129 | 27 | 360 |
| | | | Intron 9 | | | | | |
| 532963 | n/a | n/a | Intron 13 | TGCTCCTCACCCCAGTCCTC | 6593 | 6612 | 24 | 361 |
| 532964 | n/a | n/a | Intron 15- | TCTCACAGCTGCCTTTCTGT | 7115 | 7134 | 25 | 362 |
| | | | Exon 16 | | | | | |
| 532965 | n/a | n/a | Exon 17- | GAAAGGGAGGACTCACTTGA | 7379 | 7398 | 11 | 363 |
| | | | Intron 17 | | | | | |
| 532966 | n/a | n/a | Intron 7 | CCATCTTTTAACCCCAGAGA | 4545 | 4564 | 18 | 364 |
| 532967 | n/a | n/a | Intron 13 | TCCTCACCCCAGTCCTCCAG | 6590 | 6609 | 27 | 365 |
| 532968 | n/a | n/a | Intron 10 | CTGGCAGAGAAGGAGAATGT | 5808 | 5827 | 15 | 366 |
| 532969 | n/a | n/a | Intron 17 | TCTCCCCAGATAGGAAAGGG | 7392 | 7411 | 23 | 367 |
| 532970 | n/a | n/a | Intron 14 | ACTTCAGCTGCTCCCACCAC | 6882 | 6901 | 18 | 368 |
| 532971 | n/a | n/a | Intron 1 | GACAGCAGGAAGGGAAGGTT | 2076 | 2095 | 13 | 369 |
| 532972 | n/a | n/a | Intron 13- | GGAGACAAATGGGCCTATAA | 6640 | 6659 | 33 | 370 |
| | | | Exon 14 | | | | | |
| 532973 | n/a | n/a | Intron 14 | CTGCTCCCACCACTGTCATC | 6875 | 6894 | 11 | 371 |
| 532974 | n/a | n/a | Intron 10 | AGGAATGAAGAAGGCTTTCC | 5428 | 5447 | 21 | 372 |
| 532975 | n/a | n/a | Intron 14 | GGGATCTCATCCTTATCCTC | 6741 | 6760 | 31 | 373 |
| 532976 | n/a | n/a | Intron 9 | GTGCTGGGTCCTAGGCAGGT | 5130 | 5149 | 16 | 374 |
| 532977 | n/a | n/a | Intron 1 | CAAAAGGCCAAGGAGGGATG | 2101 | 2120 | 14 | 375 |
| 532978 | n/a | n/a | Intron 17 | CCATGCTGACCACTTGGCAT | 7411 | 7430 | 20 | 376 |
| 532979 | n/a | n/a | Intron 8 | GGAGGCTGGGACAGGAGAGA | 4753 | 4772 | 25 | 377 |
| 532980 | n/a | n/a | Intron 14- | GGAGCAGCTCTTCCTCTGGA | 6917 | 6936 | 36 | 378 |
| | | | Exon 15 | | | | | |
| 532981 | n/a | n/a | Exon 3- | TCTCACCTCCGTTGTCACAG | 2957 | 2976 | 20 | 379 |
| | | | Intron 3 | | | | | |
| 532982 | n/a | n/a | Intron 13 | CAGTCCTCCAGCCTTTCCCA | 6581 | 6600 | 21 | 380 |
| 532983 | n/a | n/a | Intron 13 | AGTCCTCCAGCCTTTCCCAG | 6580 | 6599 | 22 | 381 |
| 532984 | n/a | n/a | Intron 4- | TGAAGGAGTCTGGGAGAGTC | 3509 | 3528 | 12 | 382 |
| | | | Exon 5 | | | | | |
| 532985 | n/a | n/a | Intron 16- | CAGAATCACCTGCAAGGAGA | 7322 | 7341 | 20 | 383 |
| | | | Exon 17 | | | | | |
| 532986 | n/a | n/a | Exon 17- | TAGGAAAGGGAGGACTCACT | 7382 | 7401 | 3 | 384 |
| | | | Intron 17 | | | | | |
| 532987 | n/a | n/a | Exon 4- | ACCTTGGCAGGAAGGCTCCG | 3282 | 3301 | 12 | 385 |
| | | | Intron 4 | | | | | |
| 532988 | n/a | n/a | Intron 13- | GAGACAAATGGGCCTATAAA | 6639 | 6658 | 15 | 386 |
| | | | Exon 14 | | | | | |
| 532989 | n/a | n/a | Intron 1 | CTGAAGAGAAAGGCTGATGA | 2131 | 2150 | 17 | 387 |
| 532990 | n/a | n/a | Intron 6 | AATGATCAGGGAGCTAGTCC | 3913 | 3932 | 30 | 388 |
| 532991 | n/a | n/a | Intron 17 | CTTAGCTGACCTAAAGGAAT | 7557 | 7576 | 22 | 389 |
| 532992 | n/a | n/a | Intron 8 | TGGGTATAGTGTTACAAGTG | 4807 | 4826 | 17 | 390 |
| 532993 | n/a | n/a | Intron 1 | TGAAGAGAAAGGCTGATGAA | 2130 | 2149 | 19 | 391 |
| 532994 | n/a | n/a | Intron 8 | GTGTTACAAGTGGACTTAAG | 4799 | 4818 | 25 | 392 |
| 532995 | n/a | n/a | Intron 6 | ACCTGTGGGTGAGGAGAACA | 4134 | 4153 | 24 | 393 |
| 532996 | n/a | n/a | Exon 9- | TCACCCAGATAATCCTCCCT | 5107 | 5126 | 36 | 394 |
| | | | Intron 9 | | | | | |
| 532952 | 2608 | 2627 | Exon 18 | TGTTGTCGCAGCTGTTTTAA | 7843 | 7862 | 90 | 395 |

### Example 116: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. Cultured HepG2 cells at a density of 20,000 cells per well were transfected using electroporation with 4,500 nM antisense oligonucleotide. After a treatment period of approximately 24 hours, RNA was isolated from the cells and CFB mRNA levels were measured by quantitative real-time PCR. Human primer probe set RTS3460_MGB (forward sequence CGAAGCAGCTCAATGAAATCAA, designated herein as SEQ ID NO: 813; reverse sequence TGCCTGGAGGGCCTTCTT, designated herein as SEQ ID NO: 814; probe sequence AGACCACAAGTTGAAGTC, designated herein as SEQ ID NO: 815) was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 5-10-5 MOE gapmers. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 10000 complementarity.

TABLE 129
Inhibition of CFB mRNA by 5-10-5 MOE gapmers targeting SEQ ID NO: 1 and 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | Region | Sequence | bition | site | site | NO: |
| 532686 | 1135 | 1154 | Exon 6 | ACACTTTTTGGCTCCTGTGA | 48 | 3819 | 3838 | 84 |
| 532687 | 1141 | 1160 | Exon 6 | GACTAGACACTTTTTGGCTC | 63 | 3825 | 3844 | 85 |
| 532688 | 1147 | 1166 | Exon 6 | TAAGTTGACTAGACACTTTT | 47 | 3831 | 3850 | 86 |
| 532689 | 1153 | 1172 | Exon 6 | CTCAATTAAGTTGACTAGAC | 57 | 3837 | 3856 | 87 |
| 532690 | 1159 | 1178 | Exon 6-7 | CACCTTCTCAATTAAGTTGA | 49 | 3843 | 3862 | 88 |
| | | | Junction | | | | | |
| 532691 | 1165 | 1184 | Exon 6-7 | ACTTGCCACCTTCTCAATTA | 33 | n/a | n/a | 89 |
| | | | Junction | | | | | |
| 532692 | 1171 | 1190 | Exon 6-7 | ACCATAACTTGCCACCTTCT | 67 | n/a | n/a | 90 |
| | | | Junction | | | | | |
| 532693 | 1177 | 1196 | Exon 7 | CTTCACACCATAACTTGCCA | 56 | 4153 | 4172 | 91 |
| 532694 | 1183 | 1202 | Exon 7 | TCTTGGCTTCACACCATAAC | 50 | 4159 | 4178 | 92 |
| 532695 | 1208 | 1227 | Exon 7 | ATGTGGCATATGTCACTAGA | 53 | 4184 | 4203 | 93 |
| 532696 | 1235 | 1254 | Exon 7 | CAGACACTTTGACCCAAATT | 52 | 4211 | 4230 | 94 |
| 532697 | 1298 | 1317 | Exon 7-8 | GGTCTTCATAATTGATTTCA | 59 | n/a | n/a | 95 |
| | | | Juncion | | | | | |
| 532698 | 1304 | 1323 | Exon 7-8 | ACTTGTGGTCTTCATAATTG | 52 | n/a | n/a | 96 |
| | | | Juncion | | | | | |
| 532699 | 1310 | 1329 | Exon 7-8 | ACTTCAACTTGTGGTCTTCA | 85 | n/a | n/a | 97 |
| | | | Juncion | | | | | |
| 532700 | 1316 | 1335 | Exon 8 | TCCCTGACTTCAACTTGTGG | 96 | 4609 | 4628 | 98 |
| 532701 | 1322 | 1341 | Exon 8 | TGTTAGTCCCTGACTTCAAC | 56 | 4615 | 4634 | 99 |
| 532702 | 1328 | 1347 | Exon 8 | TCTTGGTGTTAGTCCCTGAC | 86 | 4621 | 4640 | 100 |
| 532703 | 1349 | 1368 | Exon 8 | TGTACACTGCCTGGAGGGCC | 35 | 4642 | 4661 | 101 |
| 532704 | 1355 | 1374 | Exon 8 | TCATGCTGTACACTGCCTGG | 12 | 4648 | 4667 | 102 |
| 532705 | 1393 | 1412 | Exon 8 | GTTCCAGCCTTCAGGAGGGA | 27 | 4686 | 4705 | 103 |
| 532706 | 1399 | 1418 | Exon 8 | GGTGCGGTTCCAGCCTTCAG | 67 | 4692 | 4711 | 104 |
| 532707 | 1405 | 1424 | Exon 8 | ATGGCGGGTGCGGTTCCAGC | 26 | 4698 | 4717 | 105 |
| 532708 | 1411 | 1430 | Exon 8 | GATGACATGGCGGGTGCGGT | 28 | 4704 | 4723 | 106 |
| 532709 | 1417 | 1436 | Exon 8 | GAGGATGATGACATGGCGGG | 6 | 4710 | 4729 | 107 |
| 532710 | 1443 | 1462 | Exon 8-9 | CCCATGTTGTGCAATCCATC | 35 | n/a | n/a | 108 |
| | | | Junction | | | | | |
| 532711 | 1449 | 1468 | Exon 9 | TCCCCGCCCATGTTGTGCAA | 28 | 5023 | 5042 | 109 |
| 532712 | 1455 | 1474 | Exon 9 | ATTGGGTCCCCGCCCATGTT | 19 | 5029 | 5048 | 110 |
| 532713 | 1461 | 1480 | Exon 9 | ACAGTAATTGGGTCCCCGCC | 29 | 5035 | 5054 | 111 |
| 532714 | 1467 | 1486 | Exon 9 | TCAATGACAGTAATTGGGTC | 49 | 5041 | 5060 | 112 |
| 532715 | 1473 | 1492 | Exon 9 | ATCTCATCAATGACAGTAAT | 45 | 5047 | 5066 | 113 |
| 532716 | 1479 | 1498 | Exon 9 | TCCCGGATCTCATCAATGAC | 54 | 5053 | 5072 | 114 |
| 532717 | 1533 | 1552 | Exon 9- | ACATCCAGATAATCCTCCCT | 22 | n/a | n/a | 115 |
| | | | 10 | | | | | |
| | | | Junction | | | | | |
| 532718 | 1539 | 1558 | Exon 9- | ACATAGACATCCAGATAATC | 8 | n/a | n/a | 116 |
| | | | 10 | | | | | |
| | | | Junction | | | | | |
| 532719 | 1545 | 1564 | Exon 9- | CCAAACACATAGACATCCAG | 30 | n/a | n/a | 117 |
| | | | 10 | | | | | |
| | | | Junction | | | | | |
| 532720 | 1582 | 1601 | Exon 10 | AGCATTGATGTTCACTTGGT | 62 | 5231 | 5250 | 118 |
| 532721 | 1588 | 1607 | Exon 10 | AGCCAAAGCATTGATGTTCA | 46 | 5237 | 5256 | 119 |
| 532722 | 1594 | 1613 | Exon 10 | CTTGGAAGCCAAAGCATTGA | 35 | 5243 | 5262 | 120 |
| 532723 | 1600 | 1619 | Exon 10 | GTCTTTCTTGGAAGCCAAAG | 43 | 5249 | 5268 | 121 |
| 532724 | 1606 | 1625 | Exon 10 | CTCATTGTCTTTCTTGGAAG | 40 | 5255 | 5274 | 122 |
| 532725 | 1612 | 1631 | Exon 10 | ATGTTGCTCATTGTCTTTCT | 49 | 5261 | 5280 | 123 |
| 532726 | 1618 | 1637 | Exon 10 | GAACACATGTTGCTCATTGT | 68 | 5267 | 5286 | 124 |
| 532727 | 1624 | 1643 | Exon 10 | GACTTTGAACACATGTTGCT | 54 | 5273 | 5292 | 125 |
| 532728 | 1630 | 1649 | Exon 10 | ATCCTTGACTTTGAACACAT | 61 | 5279 | 5298 | 126 |
| 532729 | 1636 | 1655 | Exon 10 | TTCCATATCCTTGACTTTGA | 55 | 5285 | 5304 | 127 |
| 532730 | 1642 | 1661 | Exon 10 | CAGGTTTTCCATATCCTTGA | 51 | 5291 | 5310 | 440 |
| 532731 | 1686 | 1705 | Exon 10- | CTCAGAGACTGGCTTTCATC | 41 | 5827 | 5846 | 129 |
| | | | 11 | | | | | |
| | | | Junction | | | | | |
| 532732 | 1692 | 1711 | Exon 11 | CAGAGACTCAGAGACTGGCT | 59 | 5833 | 5852 | 130 |
| 516252 | 1698 | 1717 | Exon 11 | ATGCCACAGAGACTCAGAGA | 57 | 5839 | 5858 | 131 |
| 532733 | 1704 | 1723 | Exon 11 | CAAACCATGCCACAGAGACT | 34 | 5845 | 5864 | 132 |
| 532734 | 1710 | 1729 | Exon 11 | TGTTCCCAAACCATGCCACA | 51 | 5851 | 5870 | 133 |
| 532735 | 1734 | 1753 | Exon 11 | TTGTGGTAATCGGTACCCTT | 50 | 5875 | 5894 | 134 |
| 532736 | 1740 | 1759 | Exon 11 | GGTTGCTTGTGGTAATCGGT | 64 | 5881 | 5900 | 135 |
| 532737 | 1746 | 1765 | Exon 11 | TGCCATGGTTGCTTGTGGTA | 40 | 5887 | 5906 | 136 |
| 532738 | 1752 | 1771 | Exon 11 | TTGGCCTGCCATGGTTGCTT | 49 | 5893 | 5912 | 137 |
| 532739 | 1758 | 1777 | Exon 11 | GAGATCTTGGCCTGCCATGG | 47 | 5899 | 5918 | 138 |
| 532740 | 1803 | 1822 | Exon 12 | ACAGCCCCCATACAGCTCTC | 48 | 6082 | 6101 | 139 |
| 532741 | 1809 | 1828 | Exon 12 | GACACCACAGCCCCCATACA | 40 | 6088 | 6107 | 140 |
| 532742 | 1815 | 1834 | Exon 12 | TACTCAGACACCACAGCCCC | 33 | 6094 | 6113 | 141 |
| 532743 | 1821 | 1840 | Exon 12 | ACAAAGTACTCAGACACCAC | 39 | 6100 | 6119 | 142 |
| 532744 | 1827 | 1846 | Exon 12 | GTCAGCACAAAGTACTCAGA | 45 | 6106 | 6125 | 143 |
| 532745 | 1872 | 1891 | Exon 12 | TTGATTGAGTGTTCCTTGTC | 42 | 6151 | 6170 | 144 |
| 532746 | 1878 | 1897 | Exon 12 | CTGACCTTGATTGAGTGTTC | 53 | 6157 | 6176 | 145 |
| 532747 | 1909 | 1928 | Exon 13 | TATCTCCAGGTCCCGCTTCT | 31 | 6403 | 6422 | 146 |
| 532748 | 1967 | 1986 | Exon 13 | GAATTCCTGCTTCTTTTTTC | 30 | 6461 | 6480 | 147 |
| 532749 | 1973 | 1992 | Exon 13 | ATTCAGGAATTCCTGCTTCT | 40 | 6467 | 6486 | 148 |
| 532750 | 1979 | 1998 | Exon 13 | CATAAAATTCAGGAATTCCT | 45 | 6473 | 6492 | 149 |
| 532751 | 1985 | 2004 | Exon 13 | CATAGTCATAAAATTCAGGA | 43 | 6479 | 6498 | 150 |
| 532752 | 2006 | 2025 | Exon 13 | TGAGCTTGATCAGGGCAACG | 61 | 6500 | 6519 | 151 |
| 532753 | 2012 | 2031 | Exon 13 | TATTCTTGAGCTTGATCAGG | 47 | 6506 | 6525 | 152 |
| 532754 | 2048 | 2067 | Exon 13- | GACAAATGGGCCTGATAGTC | 35 | n/a | n/a | 153 |
| | | | 14 | | | | | |
| | | | Junction | | | | | |
| 532755 | 2070 | 2089 | Exon 14 | GTTGTTCCCTCGGTGCAGGG | 43 | 6659 | 6678 | 154 |
| 532756 | 2076 | 2095 | Exon 14 | GCTCGAGTTGTTCCCTCGGT | 51 | 6665 | 6684 | 155 |
| 532757 | 2082 | 2101 | Exon 14 | CTCAAAGCTCGAGTTGTTCC | 36 | 6671 | 6690 | 156 |
| 532758 | 2088 | 2107 | Exon 14 | GGAAGCCTCAAAGCTCGAGT | 54 | 6677 | 6696 | 157 |
| 532759 | 2094 | 2113 | Exon 14 | GTTGGAGGAAGCCTCAAAGC | 52 | 6683 | 6702 | 158 |
| 532760 | 2100 | 2119 | Exon 14 | GTGGTAGTTGGAGGAAGCCT | 22 | 6689 | 6708 | 159 |
| 532761 | 2106 | 2125 | Exon 14 | TGGCAAGTGGTAGTTGGAGG | 34 | 6695 | 6714 | 160 |
| 532762 | 2112 | 2131 | Exon 14 | TGTTGCTGGCAAGTGGTAGT | 52 | 6701 | 6720 | 161 |

### Example 117: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 5,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 5-10-5 MOE gapmers. The gapmers are 20 nucleosides in length, wherein the central gap segment comprises of ten 2′-deoxynucleosides and is flanked by wing segments on the 5′ direction and the 3′ direction comprising five nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines. “Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity. In case the sequence alignment for a target gene in a particular table is not shown, it is understood that none of the oligonucleotides presented in that table align with 100% complementarity with that target gene.

TABLE 130
Inhibition of CFB mRNA by 5-10-5 MOE gapmers targeting SEQ ID NO: 1

| | SEQ ID | SEQ ID | | | | |
|---|---|---|---|---|---|---|
| | NO: 1 | NO: 1 | | | % | SEQ |
| ISIS | start | stop | Target | | inhi- | ID |
| NO | site | site | Region | Sequence | bition | NO: |
| 588570 | 150 | 169 | Exon 1 | TGGTCACATTCCCTTCCCCT | 54 | 396 |
| 588571 | 152 | 171 | Exon 1 | CCTGGTCACATTCCCTTCCC | 63 | 397 |
| 532614 | 154 | 173 | Exon 1 | GACCTGGTCACATTCCCTTC | 64 | 12 |
| 588572 | 156 | 175 | Exon 1 | TAGACCTGGTCACATTCCCT | 62 | 398 |
| 588573 | 158 | 177 | Exon 1 | CCTAGACCTGGTCACATTCC | 53 | 399 |
| 588566 | 2189 | 2208 | Exon 15 | CCTTCCGAGTCAGCTTTTTC | 60 | 400 |
| 588567 | 2191 | 2210 | Exon 15 | CTCCTTCCGAGTCAGCTTTT | 61 | 401 |
| 532770 | 2193 | 2212 | Exon 15 | ACCTCCTTCCGAGTCAGCTT | 77 | 198 |
| 588568 | 2195 | 2214 | Exon 15 | AGACCTCCTTCCGAGTCAGC | 72 | 402 |
| 588569 | 2197 | 2216 | Exon 15 | GTAGACCTCCTTCCGAGTCA | 46 | 403 |
| 588574 | 2453 | 2472 | Exon 18 | TTTGCCGCTTCTGGTTTTTG | 46 | 404 |
| 588575 | 2455 | 2474 | Exon 18 | CTTTTGCCGCTTCTGGTTTT | 41 | 405 |
| 532800 | 2457 | 2476 | Exon 18 | TGCTTTTGCCGCTTCTGGTT | 69 | 228 |
| 588576 | 2459 | 2478 | Exon 18 | CCTGCTTTTGCCGCTTCTGG | 61 | 406 |
| 588577 | 2461 | 2480 | Exon 18 | TACCTGCTTTTGCCGCTTCT | 51 | 407 |
| 516350 | 2550 | 2569 | Exon 18 | AGAAAACCCAAATCCTCATC | 71 | 408 |
| 588509 | 2551 | 2570 | Exon 18 | TAGAAAACCCAAATCCTCAT | 58 | 409 |
| 588510 | 2552 | 2571 | Exon 18 | ATAGAAAACCCAAATCCTCA | 57 | 410 |
| 588511 | 2553 | 2572 | Exon 18 | TATAGAAAACCCAAATCCTC | 57 | 411 |
| 588512 | 2554 | 2573 | Exon 18 | TTATAGAAAACCCAAATCCT | 44 | 412 |
| 588513 | 2555 | 2574 | Exon 18 | CTTATAGAAAACCCAAATCC | 37 | 413 |
| 588514 | 2556 | 2575 | Exon 18 | CCTTATAGAAAACCCAAATC | 50 | 414 |
| 588515 | 2557 | 2576 | Exon 18 | CCCTTATAGAAAACCCAAAT | 45 | 415 |
| 588516 | 2558 | 2577 | Exon 18 | CCCCTTATAGAAAACCCAAA | 60 | 416 |
| 588517 | 2559 | 2578 | Exon 18 | ACCCCTTATAGAAAACCCAA | 67 | 417 |
| 588518 | 2560 | 2579 | Exon 18 | AACCCCTTATAGAAAACCCA | 57 | 418 |
| 588519 | 2561 | 2580 | Exon 18 | AAACCCCTTATAGAAAACCC | 61 | 419 |
| 588520 | 2562 | 2581 | Exon 18 | GAAACCCCTTATAGAAAACC | 27 | 420 |
| 588521 | 2563 | 2582 | Exon 18 | GGAAACCCCTTATAGAAAAC | 25 | 421 |
| 588522 | 2564 | 2583 | Exon 18 | AGGAAACCCCTTATAGAAAA | 36 | 422 |
| 588523 | 2565 | 2584 | Exon 18 | CAGGAAACCCCTTATAGAAA | 36 | 423 |
| 588524 | 2566 | 2585 | Exon 18 | GCAGGAAACCCCTTATAGAA | 46 | 424 |
| 588525 | 2567 | 2586 | Exon 18 | AGCAGGAAACCCCTTATAGA | 38 | 425 |
| 588526 | 2568 | 2587 | Exon 18 | CAGCAGGAAACCCCTTATAG | 47 | 426 |
| 588527 | 2569 | 2588 | Exon 18 | CCAGCAGGAAACCCCTTATA | 68 | 427 |
| 588528 | 2570 | 2589 | Exon 18 | TCCAGCAGGAAACCCCTTAT | 63 | 428 |
| 532809 | 2571 | 2590 | Exon 18 | GTCCAGCAGGAAACCCCTTA | 85 | 237 |
| 588529 | 2572 | 2591 | Exon 18 | TGTCCAGCAGGAAACCCCTT | 76 | 429 |
| 588530 | 2573 | 2592 | Exon 18 | CTGTCCAGCAGGAAACCCCT | 74 | 430 |
| 588531 | 2574 | 2593 | Exon 18 | CCTGTCCAGCAGGAAACCCC | 75 | 431 |
| 588532 | 2575 | 2594 | Exon 18 | CCCTGTCCAGCAGGAAACCC | 73 | 432 |
| 588533 | 2576 | 2595 | Exon 18 | CCCCTGTCCAGCAGGAAACC | 82 | 433 |
| 532810 | 2577 | 2596 | Exon 18 | GCCCCTGTCCAGCAGGAAAC | 88 | 238 |
| 588534 | 2578 | 2597 | Exon 18 | CGCCCCTGTCCAGCAGGAAA | 86 | 434 |
| 588535 | 2579 | 2598 | Exon 18 | ACGCCCCTGTCCAGCAGGAA | 86 | 435 |
| 588536 | 2580 | 2599 | Exon 18 | CACGCCCCTGTCCAGCAGGA | 93 | 436 |
| 588537 | 2581 | 2600 | Exon 18 | CCACGCCCCTGTCCAGCAGG | 92 | 437 |
| 588538 | 2582 | 2601 | Exon 18 | CCCACGCCCCTGTCCAGCAG | 94 | 438 |
| 588539 | 2583 | 2602 | Exon 18 | TCCCACGCCCCTGTCCAGCA | 96 | 439 |
| 588540 | 2584 | 2603 | Exon 18 | ATCCCACGCCCCTGTCCAGC | 88 | 440 |
| 588541 | 2585 | 2604 | Exon 18 | AATCCCACGCCCCTGTCCAG | 79 | 441 |
| 588542 | 2586 | 2605 | Exon 18 | CAATCCCACGCCCCTGTCCA | 83 | 442 |
| 588543 | 2587 | 2606 | Exon 18 | TCAATCCCACGCCCCTGTCC | 86 | 443 |
| 588544 | 2588 | 2607 | Exon 18 | TTCAATCCCACGCCCCTGTC | 90 | 444 |
| 588545 | 2589 | 2608 | Exon 18 | ATTCAATCCCACGCCCCTGT | 92 | 445 |
| 588546 | 2590 | 2609 | Exon 18 | AATTCAATCCCACGCCCCTG | 92 | 446 |
| 588547 | 2591 | 2610 | Exon 18 | TAATTCAATCCCACGCCCCT | 88 | 447 |
| 588548 | 2592 | 2611 | Exon 18 | TTAATTCAATCCCACGCCCC | 93 | 448 |
| 588549 | 2593 | 2612 | Exon 18 | TTTAATTCAATCCCACGCCC | 88 | 449 |
| 588550 | 2594 | 2613 | Exon 18 | TTTTAATTCAATCCCACGCC | 89 | 450 |
| 588551 | 2595 | 2614 | Exon 18 | GTTTTAATTCAATCCCACGC | 94 | 451 |
| 588552 | 2596 | 2615 | Exon 18 | TGTTTTAATTCAATCCCACG | 93 | 452 |
| 588553 | 2597 | 2616 | Exon 18 | CTGTTTTAATTCAATCCCAC | 96 | 453 |
| 588554 | 2598 | 2617 | Exon 18 | GCTGTTTTAATTCAATCCCA | 98 | 454 |
| 532811 | 2599 | 2618 | Exon 18 | AGCTGTTTTAATTCAATCCC | 97 | 239 |
| 532811 | 2599 | 2618 | Exon 18 | AGCTGTTTTAATTCAATCCC | 95 | 239 |
| 588555 | 2600 | 2619 | Exon 18 | CAGCTGTTTTAATTCAATCC | 93 | 455 |
| 588556 | 2601 | 2620 | Exon 18 | GCAGCTGTTTTAATTCAATC | 96 | 456 |
| 588557 | 2602 | 2621 | Exon 18 | CGCAGCTGTTTTAATTCAAT | 98 | 457 |
| 588558 | 2603 | 2622 | Exon 18 | TCGCAGCTGTTTTAATTCAA | 95 | 458 |
| 532917 | 2604 | 2623 | Exon 18 | GTCGCAGCTGTTTTAATTCA | 97 | 317 |
| 588559 | 2605 | 2624 | Exon 18 | TGTCGCAGCTGTTTTAATTC | 95 | 459 |
| 588560 | 2606 | 2625 | Exon 18 | TTGTCGCAGCTGTTTTAATT | 92 | 460 |
| 588561 | 2607 | 2626 | Exon 18 | GTTGTCGCAGCTGTTTTAAT | 93 | 461 |
| 532952 | 2608 | 2627 | Exon 18 | TGTTGTCGCAGCTGTTTTAA | 88 | 395 |
| 588562 | 2609 | 2628 | Exon 18/ | TTGTTGTCGCAGCTGTTTTA | 90 | 462 |
| | | | Repeat | | | |
| 588563 | 2610 | 2629 | Exon 18/ | TTTGTTGTCGCAGCTGTTTT | 89 | 463 |
| | | | Repeat | | | |
| 588564 | 2611 | 2630 | Exon 18/ | TTTTGTTGTCGCAGCTGTTT | 92 | 464 |
| | | | Repeat | | | |
| 588565 | 2612 | 2631 | Exon 18/ | TTTTTGTTGTCGCAGCTGTT | 88 | 465 |
| | | | Repeat | | | |

TABLE 131
Inhibition of CFB mRNA by 5-10-5 MOE gapmers targeting SEQ ID NO: 1 or
SEQ ID NO: 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | Region | Sequence | bition | site | site | NO: |
| 588685 | n/a | n/a | Exon 1 | GGATCCAGCTCACTCCCCTG | 48 | 1596 | 1615 | 466 |
| 588686 | n/a | n/a | Exon 1 | AAATAAGGATCCAGCTCACT | 29 | 1602 | n/a | 467 |
| 588688 | n/a | n/a | Exon 1 | GACCAGAAATAAGGATCCAG | 58 | 1608 | 1627 | 468 |
| 588690 | n/a | n/a | Exon 1 | CTTAGGGACCAGAAATAAGG | 45 | 1614 | 1633 | 469 |
| 588692 | n/a | n/a | Exon 1 | CACCCACTTAGGGACCAGAA | 36 | 1620 | 1639 | 470 |
| 588694 | n/a | n/a | Exon 1 | ACCACCCACTTAGGGACCAG | 47 | 1622 | 1641 | 471 |
| 588696 | n/a | n/a | Exon 1 | AGGTCCAGGACTCTCCCCTT | 96 | 1685 | 1704 | 472 |
| 588698 | n/a | n/a | Exon 1 | AAGGTCCAGGACTCTCCCCT | 96 | 1686 | 1705 | 473 |
| 588700 | n/a | n/a | Exon 1 | AAACTGCAGAAGTCCCACCC | 2 | 1716 | 1735 | 474 |
| 588586 | 30 | 49 | Exon 1 | GGAGGGCCCCGCTGAGCTGC | 59 | 1751 | 1770 | 475 |
| 588587 | 48 | 67 | Exon 1 | TCCCGGAACATCCAAGCGGG | 45 | 1769 | 1788 | 476 |
| 588588 | 56 | 75 | Exon 1 | CATCACTTTCCCGGAACATC | 39 | 1777 | n/a | 477 |
| 588589 | 151 | 170 | Exon 1 | CTGGTCACATTCCCTTCCCC | 29 | 1872 | 1891 | 478 |
| 588590 | 157 | 176 | Exon 1 | CTAGACCTGGTCACATTCCC | 47 | 1878 | 1897 | 479 |
| 588591 | 339 | 358 | Exon 1-2 | GGAGTGGTGGTCACACCTCC | 44 | n/a | n/a | 480 |
| | | | Junction | | | | | |
| 588592 | 384 | 403 | Exon 2 | ACCCCCTCCAGAGAGCAGGA | 43 | 2192 | 2211 | 481 |
| 588593 | 390 | 409 | Exon 2 | ATCTCTACCCCCTCCAGAGA | 34 | 2198 | 2217 | 482 |
| 588594 | 467 | 486 | Exon 2 | GGTACGGGTAGAAGCCAGAA | 17 | 2275 | 2294 | 483 |
| 588595 | 671 | 690 | Exon 3 | GGAGAGTGTAACCGTCATAG | 37 | 2879 | 2898 | 484 |
| 588596 | 689 | 708 | Exon 3 | TGCGATTGGCAGAGCCCCGG | 18 | 2897 | 2916 | 485 |
| 588597 | 695 | 714 | Exon 3 | GGCAGGTGCGATTGGCAGAG | 32 | 2903 | 2922 | 486 |
| 588598 | 707 | 726 | Exon 3 | GGCCATTCACTTGGCAGGTG | 45 | 2915 | 2934 | 487 |
| 588599 | 738 | 757 | Exon 3 | TTGTCACAGATCGCTGTCTG | 52 | 2946 | 2965 | 488 |
| 588600 | 924 | 943 | Exon 4-5 | AAGGAGTCTTGGCAGGAAGG | 39 | n/a | n/a | 489 |
| | | | Junction | | | | | |
| 588601 | 931 | 950 | Exon 4-5 | GTACATGAAGGAGTCTTGGC | 37 | n/a | n/a | 490 |
| | | | Junction | | | | | |
| 588602 | 959 | 978 | Exon 5 | AAGCTTCGGCCACCTCTTGA | 21 | 3542 | 3561 | 491 |
| 588603 | 1089 | 1108 | Exon 6 | CCATCTAGCACCAGGTAGAT | 22 | 3773 | 3792 | 492 |
| 588604 | 1108 | 1127 | Exon 6 | GGCCCCAATGCTGTCTGATC | 21 | 3792 | 3811 | 493 |
| 588606 | 1150 | 1169 | Exon 6 | AATTAAGTTGACTAGACACT | 56 | 3834 | 3853 | 494 |
| 588608 | 1162 | 1181 | Exon 6-7 | TGCCACCTTCTCAATTAAGT | 50 | | 19 | 495 |
| | | | Junction | | | | | |
| 588578 | 1167 | 1186 | Exon 6-7 | TAACTTGCCACCTTCTCAAT | 23 | n/a | n/a | 496 |
| | | | Junction | | | | | |
| 588579 | 1169 | 1188 | Exon 6-7 | CATAACTTGCCACCTTCTCA | 23 | n/a | n/a | 497 |
| | | | Junction | | | | | |
| 532692 | 1171 | 1190 | Exon 6-7 | ACCATAACTTGCCACCTTCT | 15 | n/a | n/a | 90 |
| | | | Junction | | | | | |
| 588580 | 1173 | 1192 | Exon 6-7 | ACACCATAACTTGCCACCTT | 16 | n/a | n/a | 498 |
| | | | Junction | | | | | |
| 588581 | 1175 | 1194 | Exon 6-7 | TCACACCATAACTTGCCACC | 14 | 4151 | 4170 | 499 |
| | | | Junction | | | | | |
| 588610 | 1319 | 1338 | Exon 8 | TAGTCCCTGACTTCAACTTG | 50 | 4612 | 4631 | 500 |
| 588612 | 1325 | 1344 | Exon 8 | TGGTGTTAGTCCCTGACTTC | 47 | 4618 | 4637 | 501 |
| 588614 | 1396 | 1415 | Exon 8 | GCGGTTCCAGCCTTCAGGAG | 47 | 4689 | 4708 | 502 |
| 588616 | 1421 | 1440 | Exon 8 | TCATGAGGATGATGACATGG | 51 | 4714 | 4733 | 503 |
| 588618 | 1446 | 1465 | Exon 9 | CCGCCCATGTTGTGCAATCC | 18 | 5020 | 5039 | 504 |
| 588620 | 1458 | 1477 | Exon 9 | GTAATTGGGTCCCCGCCCAT | 40 | 5032 | 5051 | 505 |
| 588623 | 1482 | 1501 | Exon 9 | AAGTCCCGGATCTCATCAAT | 40 | 5056 | 5075 | 506 |
| 588624 | 1542 | 1561 | Exon 9-10 | AACACATAGACATCCAGATA | 45 | n/a | n/a | 507 |
| | | | Junction | | | | | |
| 588626 | 1585 | 1604 | Exon 10 | CAAAGCATTGATGTTCACTT | 43 | 5234 | 5253 | 508 |
| 588628 | 1621 | 1640 | Exon 10 | TTTGAACACATGTTGCTCAT | 45 | 5270 | 5289 | 509 |
| 588631 | 1646 | 1665 | Exon 10 | CTTCCAGGTTTTCCATATCC | 53 | 5295 | 5314 | 510 |
| 588632 | 1647 | 1666 | Exon 10 | TCTTCCAGGTTTTCCATATC | 56 | 5296 | 5315 | 511 |
| 588634 | 1689 | 1708 | Exon 11 | AGACTCAGAGACTGGCTTTC | 35 | 5830 | 5849 | 512 |
| 588636 | 1749 | 1768 | Exon 11 | GCCTGCCATGGTTGCTTGTG | 55 | 5890 | 5909 | 513 |
| 588638 | 1763 | 1782 | Exon 11 | TGACTGAGATCTTGGCCTGC | 78 | 5904 | 5923 | 514 |
| 588640 | 1912 | 1931 | Exon 13 | TTCTATCTCCAGGTCCCGCT | 95 | 6406 | 6425 | 515 |
| 588642 | 1982 | 2001 | Exon 13 | AGTCATAAAATTCAGGAATT | 44 | 6476 | 6495 | 516 |
| 588645 | 2073 | 2092 | Exon 14 | CGAGTTGTTCCCTCGGTGCA | 40 | 6662 | 6681 | 517 |
| 588646 | 2085 | 2104 | Exon 14 | AGCCTCAAAGCTCGAGTTGT | 57 | 6674 | 6693 | 518 |
| 588648 | 2091 | 2110 | Exon 14 | GGAGGAAGCCTCAAAGCTCG | 48 | 6680 | 6699 | 519 |
| 588651 | 2097 | 2116 | Exon 14 | GTAGTTGGAGGAAGCCTCAA | 40 | 6686 | 6705 | 520 |
| 588652 | 2103 | 2122 | Exon 14 | CAAGTGGTAGTTGGAGGAAG | 43 | 6692 | 6711 | 521 |
| 588654 | 2166 | 2185 | Exon 15 | TCCTCAGACACAAACAGAGC | 13 | 6954 | 6973 | 522 |
| 588656 | 2172 | 2191 | Exon 15 | TTCTCCTCCTCAGACACAAA | 55 | 6960 | 6979 | 523 |
| 588658 | 2196 | 2215 | Exon 15 | TAGACCTCCTTCCGAGTCAG | 44 | 6984 | 7003 | 524 |
| 588660 | 2202 | 2221 | Exon 15 | TTGATGTAGACCTCCTTCCG | 50 | 6990 | 7009 | 525 |
| 588582 | 2219 | 2238 | Exon 15-16 | CTTTCTTATCCCCATTCTTG | 19 | n/a | n/a | 526 |
| | | | Junction | | | | | |
| 588583 | 2221 | 2240 | Exon 15-16 | GCCTTTCTTATCCCCATTCT | 14 | n/a | n/a | 527 |
| | | | Junction | | | | | |
| 532775 | 2223 | 2242 | Exon 15-16 | CTGCCTTTCTTATCCCCATT | 3 | n/a | n/a | 203 |
| | | | Junction | | | | | |
| 588584 | 2225 | 2244 | Exon 15-16 | AGCTGCCTTTCTTATCCCCA | 18 | n/a | n/a | 528 |
| | | | Junction | | | | | |
| 588662 | 2226 | 2245 | Exon 15-16 | CAGCTGCCTTTCTTATCCCC | 27 | n/a | n/a | 529 |
| | | | Junction | | | | | |
| 588585 | 2227 | 2246 | Exon 15-16 | ACAGCTGCCTTTCTTATCCC | 59 | n/a | n/a | 530 |
| | | | Junction | | | | | |
| 588664 | 2238 | 2257 | Exon 16 | GCATCTCTCTCACAGCTGCC | 49 | 7122 | 7141 | 531 |
| 588666 | 2276 | 2295 | Exon 16 | AGATGTCCTTGACTTTGTCA | 41 | 7160 | 7179 | 532 |
| 588668 | 2330 | 2349 | Exon 16 | CAGCATAGGGACTCACTCCT | 41 | 7214 | 7233 | 533 |
| 588670 | 2361 | 2380 | Exon 16-17 | CCGCCAGAATCACCTCTGCA | 43 | n/a | n/a | 534 |
| | | | Junction | | | | | |
| 588672 | 2397 | 2416 | Exon 17 | TGAATGAAACGACTTCTCTT | 52 | 7362 | 7381 | 535 |
| 588674 | 2430 | 2449 | Exon 18 | ACATCCACTACTCCCCAGCT | 39 | 7665 | 7684 | 536 |
| 588676 | 2448 | 2467 | Exon 18 | CGCTTCTGGTTTTTGCAGAC | 69 | 7683 | 7702 | 537 |
| 588678 | 2454 | 2473 | Exon 18 | TTTTGCCGCTTCTGGTTTTT | 46 | 7689 | 7708 | 538 |
| 588680 | 2466 | 2485 | Exon 18 | GCAGGTACCTGCTTTTGCCG | 47 | 7701 | 7720 | 539 |
| 588682 | 2532 | 2551 | Exon 18 | TCTTGGAGTTTCTCCTTCAG | 58 | 7767 | 7786 | 540 |
| 532811 | 2599 | 2618 | Exon 18 | AGCTGTTTTAATTCAATCCC | 10 | 7834 | 7853 | 239 |
| 532917 | 2604 | 2623 | Exon 18 | GTCGCAGCTGTTTTAATTCA | 11 | 7839 | 7858 | 317 |

### Example 118: Antisense Inhibition of Human Complement Factor B (CFB) in HepG2 Cells by MOE Gapmers

Antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 3,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 4-8-5 MOE, 5-9-5 MOE, 5-10-5 MOE, 3-10-4 MOE, 3-10-7 MOE, 6-7-6-MOE, 6-8-6 MOE, or 5-7-5 MOE gapmers, or as deoxy, MOE, and (S)-cEt oligonucleotides.

The 4-8-5 MOE gapmers are 17 nucleosides in length, wherein the central gap segment comprises of eight 2′-deoxynucleosides and is flanked by wing segments on the 5′ direction and the 3′ direction comprising four and five nucleosides respectively. The 5-9-5 MOE gapmers are 19 nucleosides in length, wherein the central gap segment comprises of nine 2′-deoxynucleosides and is flanked by wing segments on the 5′ direction and the 3′ direction comprising five nucleosides each. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. The 5-7-5 MOE gapmers are 17 nucleosides in length, wherein the central gap segment comprises of seven 2′-deoxynucleosides and is flanked by wing segments on the 5′ direction and the 3′ direction comprising five nucleosides each. The 3-10-4 MOE gapmers are 17 nucleosides in length, wherein the central gap segment comprises of ten 2′-deoxynucleosides and is flanked by wing segments on the 5′ direction and the 3′ direction comprising three and four nucleosides respectively. The 3-10-7 MOE gapmers are 20 nucleosides in length, wherein the central gap segment comprises of ten 2′-deoxynucleosides and is flanked by wing segments on the 5′ direction and the 3′ direction comprising three and seven nucleosides respectively. The 6-7-6 MOE gapmers are 19 nucleosides in length, wherein the central gap segment comprises of seven 2′-deoxynucleosides and is flanked by wing segments on the 5′ direction and the 3′ direction comprising six nucleosides each. The 6-8-6 MOE gapmers are 20 nucleosides in length, wherein the central gap segment comprises of eight 2′-deoxynucleosides and is flanked by wing segments on the 5′ direction and the 3′ direction comprising six nucleosides each. The internucleoside linkages throughout each gapmer are phosphorothioate (P═S) linkages. All cytosine residues throughout each gapmer are 5-methylcytosines.

The deoxy, MOE and (S)-cEt oligonucleotides are 16 nucleosides in length wherein the nucleoside have either a MOE sugar modification, an (S)-cEt sugar modification, or a deoxy modification. The ‘Chemistry’ column describes the sugar modifications of each oligonucleotide. ‘k’ indicates an (S)-cEt sugar modification; ‘d’ indicates deoxyribose; and ‘e’ indicates a MOE modification.

“Start site” indicates the 5′-most nucleoside to which the gapmer is targeted in the human gene sequence. “Stop site” indicates the 3′-most nucleoside to which the gapmer is targeted human gene sequence.

Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 132
Inhibition of CFB mRNA by deoxy, MOE and (S)-cEt oligonucleotides targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | Start | stop | Target | | inhi- | Start | Stop | | ID |
| NO | site | site | region | Sequence | bition | site | site | Motif | NO: |
| 532811 | 2599 | 2618 | Exon 18 | AGCTGTTTTAATTCAATCCC | 10 | 7834 | 7853 | eeeeeddddddddddeeeee | 239 |
| 588884 | 48 | 63 | Exon 1 | GGAACATCCAAGCGGG | 79 | 1769 | 1784 | eekdddddddddddke | 541 |
| 588872 | 154 | 169 | Exon 1 | TGGTCACATTCCCTTC | 91 | 1875 | 1890 | eekdddddddddddke | 542 |
| 588873 | 156 | 171 | Exon 1 | CCTGGTCACATTCCCT | 91 | 1877 | 1892 | eekdddddddddddke | 543 |
| 588874 | 158 | 173 | Exon 1 | GACCTGGTCACATTCC | 91 | 1879 | 1894 | eekdddddddddddke | 544 |
| 588878 | 1171 | 1186 | Exon 6-7 | TAACTTGCCACCTTCT | 92 | n/a | n/a | eekdddddddddddke | 545 |
| | | | Junction | | | | | | |
| 588879 | 1173 | 1188 | Exon 6-7 | CATAACTTGCCACCTT | 94 | n/a | n/a | eekdddddddddddke | 546 |
| | | | Junction | | | | | | |
| 588880 | 1175 | 1190 | Exon 6-7 | ACCATAACTTGCCACC | 89 | 4151 | 4166 | eekdddddddddddke | 547 |
| | | | Junction | | | | | | |
| 588869 | 2193 | 2208 | Exon 15 | CCTTCCGAGTCAGCTT | 17 | 6981 | 6996 | eekdddddddddddke | 548 |
| 588870 | 2195 | 2210 | Exon 15 | CTCCTTCCGAGTCAGC | 78 | 6983 | 6998 | eekdddddddddddke | 549 |
| 588871 | 2197 | 2212 | Exon 15 | ACCTCCTTCCGAGTCA | 80 | 6985 | 7000 | eekdddddddddddke | 550 |
| 588881 | 2223 | 2238 | Exon 15- | CTTTCTTATCCCCATT | 93 | n/a | n/a | eekdddddddddddke | 551 |
| | | | 16 | | | | | | |
| | | | Junction | | | | | | |
| 588882 | 2225 | 2240 | Exon 15- | GCCTTTCTTATCCCCA | 88 | n/a | n/a | eekdddddddddddke | 552 |
| | | | 16 | | | | | | |
| | | | Junction | | | | | | |
| 588883 | 2227 | 2242 | Exon 15- | CTGCCTTTCTTATCCC | 90 | n/a | n/a | eekdddddddddddke | 553 |
| | | | 16 | | | | | | |
| | | | Junction | | | | | | |
| 588875 | 2457 | 2472 | Exon 18 | TTTGCCGCTTCTGGTT | 81 | 7692 | 7707 | eekdddddddddddke | 554 |
| 588876 | 2459 | 2474 | Exon 18 | CTTTTGCCGCTTCTGG | 95 | 7694 | 7709 | eekdddddddddddke | 555 |
| 588877 | 2461 | 2476 | Exon 18 | TGCTTTTGCCGCTTCT | 91 | 7696 | 7711 | eekdddddddddddke | 556 |
| 588807 | 2551 | 2566 | Exon 18 | AAACCCAAATCCTCAT | 82 | 7786 | 7801 | eekdddddddddddke | 557 |
| 588808 | 2553 | 2568 | Exon 18 | GAAAACCCAAATCCTC | 69 | 7788 | 7803 | eekdddddddddddke | 558 |
| 588809 | 2555 | 2570 | Exon 18 | TAGAAAACCCAAATCC | 51 | 7790 | 7805 | eekdddddddddddke | 559 |
| 588810 | 2556 | 2571 | Exon 18 | ATAGAAAACCCAAATC | 23 | 7791 | 7806 | eekdddddddddddke | 560 |
| 588811 | 2559 | 2574 | Exon 18 | CTTATAGAAAACCCAA | 13 | 7794 | 7809 | eekdddddddddddke | 561 |
| 588812 | 2560 | 2575 | Exon 18 | CCTTATAGAAAACCCA | 29 | 7795 | 7810 | eekdddddddddddke | 562 |
| 588813 | 2561 | 2576 | Exon 18 | CCCTTATAGAAAACCC | 53 | 7796 | 7811 | eekdddddddddddke | 563 |
| 588814 | 2562 | 2577 | Exon 18 | CCCCTTATAGAAAACC | 86 | 7797 | 7812 | eekdddddddddddke | 564 |
| 588815 | 2563 | 2578 | Exon 18 | ACCCCTTATAGAAAAC | 76 | 7798 | 7813 | eekdddddddddddke | 565 |
| 588816 | 2564 | 2579 | Exon 18 | AACCCCTTATAGAAAA | 33 | 7799 | 7814 | eekdddddddddddke | 566 |
| 588817 | 2565 | 2580 | Exon 18 | AAACCCCTTATAGAAA | 48 | 7800 | 7815 | eekdddddddddddke | 567 |
| 588818 | 2566 | 2581 | Exon 18 | GAAACCCCTTATAGAA | 44 | 7801 | 7816 | eekdddddddddddke | 568 |
| 588819 | 2567 | 2582 | Exon 18 | GGAAACCCCTTATAGA | 74 | 7802 | 7817 | eekdddddddddddke | 569 |
| 588820 | 2568 | 2583 | Exon 18 | AGGAAACCCCTTATAG | 68 | 7803 | 7818 | eekdddddddddddke | 570 |
| 588821 | 2569 | 2584 | Exon 18 | CAGGAAACCCCTTATA | 45 | 7804 | 7819 | eekdddddddddddke | 571 |
| 588822 | 2570 | 2585 | Exon 18 | GCAGGAAACCCCTTAT | 50 | 7805 | 7820 | eekdddddddddddke | 572 |
| 588823 | 2571 | 2586 | Exon 18 | AGCAGGAAACCCCTTA | 54 | 7806 | 7821 | eekdddddddddddke | 573 |
| 588824 | 2572 | 2587 | Exon 18 | CAGCAGGAAACCCCTT | 35 | 7807 | 7822 | eekdddddddddddke | 574 |
| 588825 | 2573 | 2588 | Exon 18 | CCAGCAGGAAACCCCT | 11 | 7808 | 7823 | eekdddddddddddke | 575 |
| 588826 | 2574 | 2589 | Exon 18 | TCCAGCAGGAAACCCC | 19 | 7809 | 7824 | eekdddddddddddke | 576 |
| 588827 | 2575 | 2590 | Exon 18 | GTCCAGCAGGAAACCC | 42 | 7810 | 7825 | eekdddddddddddke | 577 |
| 588828 | 2576 | 2591 | Exon 18 | TGTCCAGCAGGAAACC | 0 | 7811 | 7826 | eekdddddddddddke | 578 |
| 588829 | 2577 | 2592 | Exon 18 | CTGTCCAGCAGGAAAC | 49 | 7812 | 7827 | eekdddddddddddke | 579 |
| 588830 | 2578 | 2593 | Exon 18 | CCTGTCCAGCAGGAAA | 11 | 7813 | 7828 | eekdddddddddddke | 580 |
| 588831 | 2579 | 2594 | Exon 18 | CCCTGTCCAGCAGGAA | 20 | 7814 | 7829 | eekdddddddddddke | 581 |
| 588832 | 2580 | 2595 | Exon 18 | CCCCTGTCCAGCAGGA | 19 | 7815 | 7830 | eekdddddddddddke | 582 |
| 588833 | 2581 | 2596 | Exon 18 | GCCCCTGTCCAGCAGG | 12 | 7816 | 7831 | eekdddddddddddke | 583 |
| 588834 | 2582 | 2597 | Exon 18 | CGCCCCTGTCCAGCAG | 10 | 7817 | 7832 | eekdddddddddddke | 584 |
| 588835 | 2583 | 2598 | Exon 18 | ACGCCCCTGTCCAGCA | 13 | 7818 | 7833 | eekdddddddddddke | 585 |
| 588836 | 2584 | 2599 | Exon 18 | CACGCCCCTGTCCAGC | 13 | 7819 | 7834 | eekdddddddddddke | 586 |
| 588837 | 2585 | 2600 | Exon 18 | CCACGCCCCTGTCCAG | 39 | 7820 | 7835 | eekdddddddddddke | 587 |
| 588838 | 2586 | 2601 | Exon 18 | CCCACGCCCCTGTCCA | 54 | 7821 | 7836 | eekdddddddddddke | 588 |
| 588839 | 2587 | 2602 | Exon 18 | TCCCACGCCCCTGTCC | 51 | 7822 | 7837 | eekdddddddddddke | 589 |
| 588840 | 2588 | 2603 | Exon 18 | ATCCCACGCCCCTGTC | 65 | 7823 | 7838 | eekdddddddddddke | 590 |
| 588841 | 2589 | 2604 | Exon 18 | AATCCCACGCCCCTGT | 59 | 7824 | 7839 | eekdddddddddddke | 591 |
| 588842 | 2590 | 2605 | Exon 18 | CAATCCCACGCCCCTG | 70 | 7825 | 7840 | eekdddddddddddke | 592 |
| 588843 | 2591 | 2606 | Exon 18 | TCAATCCCACGCCCCT | 0 | 7826 | 7841 | eekdddddddddddke | 593 |
| 588844 | 2592 | 2607 | Exon 18 | TTCAATCCCACGCCCC | 48 | 7827 | 7842 | eekdddddddddddke | 594 |
| 588845 | 2593 | 2608 | Exon 18 | ATTCAATCCCACGCCC | 46 | 7828 | 7843 | eekdddddddddddke | 595 |
| 588846 | 2594 | 2609 | Exon 18 | AATTCAATCCCACGCC | 67 | 7829 | 7844 | eekdddddddddddke | 596 |
| 588847 | 2595 | 2610 | Exon 18 | TAATTCAATCCCACGC | 75 | 7830 | 7845 | eekdddddddddddke | 597 |
| 588848 | 2596 | 2611 | Exon 18 | TTAATTCAATCCCACG | 76 | 7831 | 7846 | eekdddddddddddke | 598 |
| 588849 | 2597 | 2612 | Exon 18 | TTTAATTCAATCCCAC | 94 | 7832 | 7847 | eekdddddddddddke | 599 |
| 588850 | 2598 | 2613 | Exon 18 | TTTTAATTCAATCCCA | 91 | 7833 | 7848 | eekdddddddddddke | 600 |
| 588851 | 2599 | 2614 | Exon 18 | GTTTTAATTCAATCCC | 91 | 7834 | 7849 | eekdddddddddddke | 601 |
| 588852 | 2600 | 2615 | Exon 18 | TGTTTTAATTCAATCC | 78 | 7835 | 7850 | eekdddddddddddke | 602 |
| 588853 | 2601 | 2616 | Exon 18 | CTGTTTTAATTCAATC | 81 | 7836 | 7851 | eekdddddddddddke | 603 |
| 588854 | 2602 | 2617 | Exon 18 | GCTGTTTTAATTCAAT | 63 | 7837 | 7852 | eekdddddddddddke | 604 |
| 588855 | 2603 | 2618 | Exon 18 | AGCTGTTTTAATTCAA | 65 | 7838 | 7853 | eekdddddddddddke | 605 |
| 588856 | 2604 | 2619 | Exon 18 | CAGCTGTTTTAATTCA | 76 | 7839 | 7854 | eekdddddddddddke | 606 |
| 588857 | 2605 | 2620 | Exon 18 | GCAGCTGTTTTAATTC | 89 | 7840 | 7855 | eekdddddddddddke | 607 |
| 588858 | 2606 | 2621 | Exon 18 | CGCAGCTGTTTTAATT | 89 | 7841 | 7856 | eekdddddddddddke | 608 |
| 588859 | 2607 | 2622 | Exon 18 | TCGCAGCTGTTTTAAT | 89 | 7842 | 7857 | eekdddddddddddke | 609 |
| 588860 | 2608 | 2623 | Exon 18 | GTCGCAGCTGTTTTAA | 76 | 7843 | 7858 | eekdddddddddddke | 610 |
| 588861 | 2609 | 2624 | Exon 18 | TGTCGCAGCTGTTTTA | 87 | 7844 | 7859 | eekdddddddddddke | 611 |
| 588862 | 2610 | 2625 | Exon 18 | TTGTCGCAGCTGTTTT | 85 | 7845 | 7860 | eekdddddddddddke | 612 |
| 588863 | 2611 | 2626 | Exon 18 | GTTGTCGCAGCTGTTT | 87 | 7846 | 7861 | eekdddddddddddke | 613 |
| 588864 | 2612 | 2627 | Exon 18 | TGTTGTCGCAGCTGTT | 67 | 7847 | 7862 | eekdddddddddddke | 614 |
| 588865 | 2613 | 2628 | Exon 18 | TTGTTGTCGCAGCTGT | 51 | n/a | n/a | eekdddddddddddke | 615 |
| 588866 | 2614 | 2629 | Exon 18 | TTTGTTGTCGCAGCTG | 95 | n/a | n/a | eekdddddddddddke | 616 |
| 588867 | 2615 | 2630 | Exon 18 | TTTTGTTGTCGCAGCT | 92 | n/a | n/a | eekdddddddddddke | 617 |
| 588868 | 2616 | 2631 | Exon 18 | TTTTTGTTGTCGCAGC | 66 | n/a | n/a | eekdddddddddddke | 618 |

TABLE 133
Inhibition of CFB mRNA by 5-10-5 MOE gapmers targeting SEQ ID NO: 1 or SEQ
ID NO: 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | Start | stop | Target | | inhi- | Start | Stop | ID |
| NO | site | site | region | Sequence | bition | site | site | NO: |
| 588685 | n/a | n/a | Exon 1 | GGATCCAGCTCACTCCCCTG | 14 | 1596 | 1615 | 466 |
| 588686 | n/a | n/a | Exon 1 | AAATAAGGATCCAGCTCACT | 2 | 1602 | 1621 | 467 |
| 588688 | n/a | n/a | Exon 1 | GACCAGAAATAAGGATCCAG | 3 | 1608 | 1627 | 468 |
| 588690 | n/a | n/a | Exon 1 | CTTAGGGACCAGAAATAAGG | 10 | 1614 | 1633 | 469 |
| 588692 | n/a | n/a | Exon 1 | CACCCACTTAGGGACCAGAA | 23 | 1620 | 1639 | 470 |
| 588694 | n/a | n/a | Exon 1 | ACCACCCACTTAGGGACCAG | 23 | 1622 | 1641 | 471 |
| 588696 | n/a | n/a | Exon 1 | AGGTCCAGGACTCTCCCCTT | 15 | 1685 | 1704 | 472 |
| 588698 | n/a | n/a | Exon 1 | AAGGTCCAGGACTCTCCCCT | 19 | 1686 | 1705 | 473 |
| 588700 | n/a | n/a | Exon 1 | AAACTGCAGAAGTCCCACCC | 16 | 1716 | 1735 | 474 |
| 588586 | 30 | 49 | Exon 1 | GGAGGGCCCCGCTGAGCTGC | 11 | 1751 | 1770 | 475 |
| 588587 | 48 | 67 | Exon 1 | TCCCGGAACATCCAAGCGGG | 14 | 1769 | 1788 | 476 |
| 588588 | 56 | 75 | Exon 1 | CATCACTTTCCCGGAACATC | 18 | 1777 | 1796 | 477 |
| 588589 | 151 | 170 | Exon 1 | CTGGTCACATTCCCTTCCCC | 59 | 1872 | 1891 | 478 |
| 588590 | 157 | 176 | Exon 1 | CTAGACCTGGTCACATTCCC | 59 | 1878 | 1897 | 479 |
| 588591 | 339 | 358 | Exon 1-2 | GGAGTGGTGGTCACACCTCC | 45 | n/a | n/a | 480 |
| | | | Junction | | | | | |
| 588592 | 384 | 403 | Exon 2 | ACCCCCTCCAGAGAGCAGGA | 39 | 2192 | 2211 | 481 |
| 588593 | 390 | 409 | Exon 2 | ATCTCTACCCCCTCCAGAGA | 29 | 2198 | 2217 | 482 |
| 588594 | 467 | 486 | Exon 2 | GGTACGGGTAGAAGCCAGAA | 47 | 2275 | 2294 | 483 |
| 588595 | 671 | 690 | Exon 3 | GGAGAGTGTAACCGTCATAG | 44 | 2879 | 2898 | 484 |
| 588596 | 689 | 708 | Exon 3 | TGCGATTGGCAGAGCCCCGG | 43 | 2897 | 2916 | 638 |
| 588597 | 695 | 714 | Exon 3 | GGCAGGTGCGATTGGCAGAG | 34 | 2903 | 2922 | 486 |
| 588598 | 707 | 726 | Exon 3 | GGCCATTCACTTGGCAGGTG | 17 | 2915 | 2934 | 487 |
| 588599 | 738 | 757 | Exon 3 | TTGTCACAGATCGCTGTCTG | 37 | 2946 | 2965 | 488 |
| 588600 | 924 | 943 | Exon 3-4 | AAGGAGTCTTGGCAGGAAGG | 18 | n/a | n/a | 489 |
| | | | Junction | | | | | |
| 588601 | 931 | 950 | Exon 3-4 | GTACATGAAGGAGTCTTGGC | 32 | n/a | n/a | 490 |
| | | | Junction | | | | | |
| 588602 | 959 | 978 | Exon 5 | AAGCTTCGGCCACCTCTTGA | 45 | 3542 | 3561 | 491 |
| 588603 | 1089 | 1108 | Exon 6 | CCATCTAGCACCAGGTAGAT | 52 | 3773 | 3792 | 492 |
| 588604 | 1108 | 1127 | Exon 6 | GGCCCCAATGCTGTCTGATC | 39 | 3792 | 3811 | 493 |
| 588606 | 1150 | 1169 | Exon 6 | AATTAAGTTGACTAGACACT | 37 | 3834 | 3853 | 494 |
| 588608 | 1162 | 1181 | Exon 6-7 | TGCCACCTTCTCAATTAAGT | 21 | n/a | n/a | 648 |
| | | | Junction | | | | | |
| 588578 | 1167 | 1186 | Exon 6-7 | TAACTTGCCACCTTCTCAAT | 22 | n/a | n/a | 496 |
| | | | Junction | | | | | |
| 588579 | 1169 | 1188 | Exon 6-7 | CATAACTTGCCACCTTCTCA | 21 | n/a | n/a | 497 |
| | | | Junction | | | | | |
| 532692 | 1171 | 1190 | Exon 6-7 | ACCATAACTTGCCACCTTCT | 56 | n/a | n/a | 90 |
| | | | Junction | | | | | |
| 588580 | 1173 | 1192 | Exon 6-7 | ACACCATAACTTGCCACCTT | 50 | n/a | n/a | 498 |
| | | | Junction | | | | | |
| 588581 | 1175 | 1194 | Exon 7 | TCACACCATAACTTGCCACC | 50 | 4151 | 4170 | 499 |
| 588610 | 1319 | 1338 | Exon 8 | TAGTCCCTGACTTCAACTTG | 47 | 4612 | 4631 | 500 |
| 588612 | 1325 | 1344 | Exon 8 | TGGTGTTAGTCCCTGACTTC | 47 | 4618 | 4637 | 501 |
| 588614 | 1396 | 1415 | Exon 8 | GCGGTTCCAGCCTTCAGGAG | 51 | 4689 | 4708 | 502 |
| 588616 | 1421 | 1440 | Exon 8 | TCATGAGGATGATGACATGG | 18 | 4714 | 4733 | 503 |
| 588618 | 1446 | 1465 | Exon 9 | CCGCCCATGTTGTGCAATCC | 40 | 5020 | 5039 | 504 |
| 588620 | 1458 | 1477 | Exon 9 | GTAATTGGGTCCCCGCCCAT | 40 | 5032 | 5051 | 505 |
| 588623 | 1482 | 1501 | Exon 9 | AAGTCCCGGATCTCATCAAT | 45 | 5056 | 5075 | 506 |
| 588624 | 1542 | 1561 | Exon 9-10 | AACACATAGACATCCAGATA | 43 | n/a | n/a | 507 |
| | | | Junction | | | | | |
| 588626 | 1585 | 1604 | Exon 10 | CAAAGCATTGATGTTCACTT | 45 | 5234 | 5253 | 508 |
| 588628 | 1621 | 1640 | Exon 10 | TTTGAACACATGTTGCTCAT | 53 | 5270 | 5289 | 509 |
| 588631 | 1646 | 1665 | Exon 10 | CTTCCAGGTTTTCCATATCC | 56 | 5295 | 5314 | 510 |
| 588632 | 1647 | 1666 | Exon 10 | TCTTCCAGGTTTTCCATATC | 35 | 5296 | 5315 | 511 |
| 588634 | 1689 | 1708 | Exon 11 | AGACTCAGAGACTGGCTTTC | 55 | 5830 | 5849 | 512 |
| 588636 | 1749 | 1768 | Exon 11 | GCCTGCCATGGTTGCTTGTG | 78 | 5890 | 5909 | 513 |
| 588638 | 1763 | 1782 | Exon 11 | TGACTGAGATCTTGGCCTGC | 95 | 5904 | 5923 | 514 |
| 588640 | 1912 | 1931 | Exon 13 | TTCTATCTCCAGGTCCCGCT | 44 | 6406 | 6425 | 515 |
| 588642 | 1982 | 2001 | Exon 13 | AGTCATAAAATTCAGGAATT | 40 | 6476 | 6495 | 516 |
| 588645 | 2073 | 2092 | Exon 14 | CGAGTTGTTCCCTCGGTGCA | 57 | 6662 | 6681 | 517 |
| 588646 | 2085 | 2104 | Exon 14 | AGCCTCAAAGCTCGAGTTGT | 48 | 6674 | 6693 | 518 |
| 588648 | 2091 | 2110 | Exon 14 | GGAGGAAGCCTCAAAGCTCG | 40 | 6680 | 6699 | 519 |
| 588651 | 2097 | 2116 | Exon 14 | GTAGTTGGAGGAAGCCTCAA | 43 | 6686 | 6705 | 520 |
| 588652 | 2103 | 2122 | Exon 14 | CAAGTGGTAGTTGGAGGAAG | 13 | 6692 | 6711 | 521 |
| 588654 | 2166 | 2185 | Exon 15 | TCCTCAGACACAAACAGAGC | 55 | 6954 | 6973 | 522 |
| 588656 | 2172 | 2191 | Exon 15 | TTCTCCTCCTCAGACACAAA | 44 | 6960 | 6979 | 523 |
| 588658 | 2196 | 2215 | Exon 15 | TAGACCTCCTTCCGAGTCAG | 50 | 6984 | 7003 | 524 |
| 588660 | 2202 | 2221 | Exon 15 | TTGATGTAGACCTCCTTCCG | 27 | 6990 | 7009 | 525 |
| 588582 | 2219 | 2238 | Exon 15- | CTTTCTTATCCCCATTCTTG | 49 | n/a | n/a | 526 |
| | | | 16 | | | | | |
| | | | Junction | | | | | |
| 588583 | 2221 | 2240 | Exon 15- | GCCTTTCTTATCCCCATTCT | 41 | n/a | n/a | 527 |
| | | | 16 | | | | | |
| | | | Junction | | | | | |
| 532775 | 2223 | 2242 | Exon 15- | CTGCCTTTCTTATCCCCATT | 41 | n/a | n/a | 203 |
| | | | 16 | | | | | |
| | | | Junction | | | | | |
| 588584 | 2225 | 2244 | Exon 15- | AGCTGCCTTTCTTATCCCCA | 43 | n/a | n/a | 528 |
| | | | 16 | | | | | |
| | | | Junction | | | | | |
| 588662 | 2226 | 2245 | Exon 15- | CAGCTGCCTTTCTTATCCCC | 52 | n/a | n/a | 529 |
| | | | 16 | | | | | |
| | | | Junction | | | | | |
| 588585 | 2227 | 2246 | Exon 15- | ACAGCTGCCTTTCTTATCCC | 39 | n/a | n/a | 530 |
| | | | 16 | | | | | |
| | | | Junction | | | | | |
| 588664 | 2238 | 2257 | Exon 16 | GCATCTCTCTCACAGCTGCC | 69 | 7122 | 7141 | 531 |
| 588666 | 2276 | 2295 | Exon 16 | AGATGTCCTTGACTTTGTCA | 46 | 7160 | 7179 | 532 |
| 588668 | 2330 | 2349 | Exon 16 | CAGCATAGGGACTCACTCCT | 47 | 7214 | 7233 | 533 |
| 588670 | 2361 | 2380 | Exon 16- | CCGCCAGAATCACCTCTGCA | 58 | n/a | n/a | 534 |
| | | | 17 | | | | | |
| | | | Junction | | | | | |
| 588672 | 2397 | 2416 | Exon 17 | TGAATGAAACGACTTCTCTT | 48 | 7362 | 7381 | 535 |
| 588674 | 2430 | 2449 | Exon 18 | ACATCCACTACTCCCCAGCT | 29 | 7665 | 7684 | 536 |
| 588676 | 2448 | 2467 | Exon 18 | CGCTTCTGGTTTTTGCAGAC | 58 | 7683 | 7702 | 537 |
| 588678 | 2454 | 2473 | Exon 18 | TTTTGCCGCTTCTGGTTTTT | 45 | 7689 | 7708 | 538 |
| 588680 | 2466 | 2485 | Exon 18 | GCAGGTACCTGCTTTTGCCG | 36 | 7701 | 7720 | 539 |
| 588682 | 2532 | 2551 | Exon 18 | TCTTGGAGTTTCTCCTTCAG | 47 | 7767 | 7786 | 540 |
| 532811 | 2599 | 2618 | Exon 18 | AGCTGTTTTAATTCAATCCC | 96 | 7834 | 7853 | 239 |
| 532917 | 2604 | 2623 | Exon 18 | GTCGCAGCTGTTTTAATTCA | 96 | 7839 | 7858 | 317 |

TABLE 134
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | Start | stop | Target | | inhi- | Start | Stop | | ID |
| NO | site | site | region | Sequence | bition | site | site | Motif | NO: |
| 598973 | 2552 | 2568 | Exon 18 | GAAAACCCAAATCCTCA | 40 | 7787 | 7803 | 3-10-4 | 619 |
| 599036 | 2552 | 2568 | Exon 18 | GAAAACCCAAATCCTCA | 18 | 7787 | 7803 | 5-7-5 | 619 |
| 598974 | 2553 | 2569 | Exon 18 | AGAAAACCCAAATCCTC | 28 | 7788 | 7804 | 3-10-4 | 620 |
| 599037 | 2553 | 2569 | Exon 18 | AGAAAACCCAAATCCTC | 19 | 7788 | 7804 | 5-7-5 | 620 |
| 598975 | 2554 | 2570 | Exon 18 | TAGAAAACCCAAATCCT | 15 | 7789 | 7805 | 3-10-4 | 621 |
| 599038 | 2554 | 2570 | Exon 18 | TAGAAAACCCAAATCCT | 32 | 7789 | 7805 | 5-7-5 | 621 |
| 598976 | 2555 | 2571 | Exon 18 | ATAGAAAACCCAAATCC | 12 | 7790 | 7806 | 3-10-4 | 622 |
| 599039 | 2555 | 2571 | Exon 18 | ATAGAAAACCCAAATCC | 7 | 7790 | 7806 | 5-7-5 | 622 |
| 598977 | 2557 | 2573 | Exon 18 | TTATAGAAAACCCAAAT | 13 | 7792 | 7808 | 3-10-4 | 623 |
| 599040 | 2557 | 2573 | Exon 18 | TTATAGAAAACCCAAAT | 13 | 7792 | 7808 | 5-7-5 | 623 |
| 598978 | 2558 | 2574 | Exon 18 | CTTATAGAAAACCCAAA | 0 | 7793 | 7809 | 3-10-4 | 624 |
| 599041 | 2558 | 2574 | Exon 18 | CTTATAGAAAACCCAAA | 0 | 7793 | 7809 | 5-7-5 | 624 |
| 598979 | 2559 | 2575 | Exon 18 | CCTTATAGAAAACCCAA | 8 | 7794 | 7810 | 3-10-4 | 625 |
| 599042 | 2559 | 2575 | Exon 18 | CCTTATAGAAAACCCAA | 19 | 7794 | 7810 | 5-7-5 | 625 |
| 598980 | 2560 | 2576 | Exon 18 | CCCTTATAGAAAACCCA | 42 | 7795 | 7811 | 3-10-4 | 626 |
| 599043 | 2560 | 2576 | Exon 18 | CCCTTATAGAAAACCCA | 10 | 7795 | 7811 | 5-7-5 | 626 |
| 598981 | 2561 | 2577 | Exon 18 | CCCCTTATAGAAAACCC | 20 | 7796 | 7812 | 3-10-4 | 627 |
| 599044 | 2561 | 2577 | Exon 18 | CCCCTTATAGAAAACCC | 12 | 7796 | 7812 | 5-7-5 | 627 |
| 598982 | 2562 | 2578 | Exon 18 | ACCCCTTATAGAAAACC | 10 | 7797 | 7813 | 3-10-4 | 628 |
| 599045 | 2562 | 2578 | Exon 18 | ACCCCTTATAGAAAACC | 3 | 7797 | 7813 | 5-7-5 | 628 |
| 598983 | 2563 | 2579 | Exon 18 | AACCCCTTATAGAAAAC | 0 | 7798 | 7814 | 3-10-4 | 629 |
| 599046 | 2563 | 2579 | Exon 18 | AACCCCTTATAGAAAAC | 18 | 7798 | 7814 | 5-7-5 | 629 |
| 598984 | 2564 | 2580 | Exon 18 | AAACCCCTTATAGAAAA | 0 | 7799 | 7815 | 3-10-4 | 630 |
| 599047 | 2564 | 2580 | Exon 18 | AAACCCCTTATAGAAAA | 7 | 7799 | 7815 | 5-7-5 | 630 |
| 598985 | 2565 | 2581 | Exon 18 | GAAACCCCTTATAGAAA | 0 | 7800 | 7816 | 3-10-4 | 631 |
| 599048 | 2565 | 2581 | Exon 18 | GAAACCCCTTATAGAAA | 9 | 7800 | 7816 | 5-7-5 | 631 |
| 598986 | 2566 | 2582 | Exon 18 | GGAAACCCCTTATAGAA | 0 | 7801 | 7817 | 3-10-4 | 632 |
| 599049 | 2566 | 2582 | Exon 18 | GGAAACCCCTTATAGAA | 18 | 7801 | 7817 | 5-7-5 | 632 |
| 598988 | 2567 | 2583 | Exon 18 | AGGAAACCCCTTATAGA | 0 | 7802 | 7818 | 3-10-4 | 633 |
| 599050 | 2567 | 2583 | Exon 18 | AGGAAACCCCTTATAGA | 8 | 7802 | 7818 | 5-7-5 | 633 |
| 598989 | 2568 | 2584 | Exon 18 | CAGGAAACCCCTTATAG | 0 | 7803 | 7819 | 3-10-4 | 634 |
| 598990 | 2569 | 2585 | Exon 18 | GCAGGAAACCCCTTATA | 8 | 7804 | 7820 | 3-10-4 | 635 |
| 598991 | 2570 | 2586 | Exon 18 | AGCAGGAAACCCCTTAT | 25 | 7805 | 7821 | 3-10-4 | 636 |
| 598992 | 2571 | 2587 | Exon 18 | CAGCAGGAAACCCCTTA | 12 | 7806 | 7822 | 3-10-4 | 637 |
| 598993 | 2572 | 2588 | Exon 18 | CCAGCAGGAAACCCCTT | 37 | 7807 | 7823 | 3-10-4 | 638 |
| 598994 | 2573 | 2589 | Exon 18 | TCCAGCAGGAAACCCCT | 29 | 7808 | 7824 | 3-10-4 | 639 |
| 598995 | 2574 | 2590 | Exon 18 | GTCCAGCAGGAAACCCC | 42 | 7809 | 7825 | 3-10-4 | 640 |
| 598996 | 2575 | 2591 | Exon 18 | TGTCCAGCAGGAAACCC | 36 | 7810 | 7826 | 3-10-4 | 641 |
| 598997 | 2576 | 2592 | Exon 18 | CTGTCCAGCAGGAAACC | 18 | 7811 | 7827 | 3-10-4 | 642 |
| 598998 | 2577 | 2593 | Exon 18 | CCTGTCCAGCAGGAAAC | 27 | 7812 | 7828 | 3-10-4 | 643 |
| 598999 | 2578 | 2594 | Exon 18 | CCCTGTCCAGCAGGAAA | 61 | 7813 | 7829 | 3-10-4 | 644 |
| 599000 | 2580 | 2596 | Exon 18 | GCCCCTGTCCAGCAGGA | 71 | 7815 | 7831 | 3-10-4 | 645 |
| 599001 | 2581 | 2597 | Exon 18 | CGCCCCTGTCCAGCAGG | 80 | 7816 | 7832 | 3-10-4 | 646 |
| 599002 | 2582 | 2598 | Exon 18 | ACGCCCCTGTCCAGCAG | 68 | 7817 | 7833 | 3-10-4 | 647 |
| 599003 | 2583 | 2599 | Exon 18 | CACGCCCCTGTCCAGCA | 71 | 7818 | 7834 | 3-10-4 | 648 |
| 599004 | 2584 | 2600 | Exon 18 | CCACGCCCCTGTCCAGC | 76 | 7819 | 7835 | 3-10-4 | 649 |
| 599005 | 2585 | 2601 | Exon 18 | CCCACGCCCCTGTCCAG | 70 | 7820 | 7836 | 3-10-4 | 650 |
| 599006 | 2586 | 2602 | Exon 18 | TCCCACGCCCCTGTCCA | 65 | 7821 | 7837 | 3-10-4 | 651 |
| 599007 | 2587 | 2603 | Exon 18 | ATCCCACGCCCCTGTCC | 60 | 7822 | 7838 | 3-10-4 | 652 |
| 599008 | 2588 | 2604 | Exon 18 | AATCCCACGCCCCTGTC | 72 | 7823 | 7839 | 3-10-4 | 653 |
| 599009 | 2589 | 2605 | Exon 18 | CAATCCCACGCCCCTGT | 79 | 7824 | 7840 | 3-10-4 | 654 |
| 599010 | 2590 | 2606 | Exon 18 | TCAATCCCACGCCCCTG | 73 | 7825 | 7841 | 3-10-4 | 655 |
| 599011 | 2591 | 2607 | Exon 18 | TTCAATCCCACGCCCCT | 79 | 7826 | 7842 | 3-10-4 | 656 |
| 599012 | 2592 | 2608 | Exon 18 | ATTCAATCCCACGCCCC | 67 | 7827 | 7843 | 3-10-4 | 657 |
| 599013 | 2593 | 2609 | Exon 18 | AATTCAATCCCACGCCC | 65 | 7828 | 7844 | 3-10-4 | 658 |
| 599014 | 2594 | 2610 | Exon 18 | TAATTCAATCCCACGCC | 74 | 7829 | 7845 | 3-10-4 | 659 |
| 599015 | 2595 | 2611 | Exon 18 | TTAATTCAATCCCACGC | 71 | 7830 | 7846 | 3-10-4 | 660 |
| 599016 | 2596 | 2612 | Exon 18 | TTTAATTCAATCCCACG | 48 | 7831 | 7847 | 3-10-4 | 661 |
| 599017 | 2597 | 2613 | Exon 18 | TTTTAATTCAATCCCAC | 34 | 7832 | 7848 | 3-10-4 | 662 |
| 599018 | 2598 | 2614 | Exon 18 | GTTTTAATTCAATCCCA | 56 | 7833 | 7849 | 3-10-4 | 663 |
| 599019 | 2599 | 2615 | Exon 18 | TGTTTTAATTCAATCCC | 60 | 7834 | 7850 | 3-10-4 | 664 |
| 599020 | 2600 | 2616 | Exon 18 | CTGTTTTAATTCAATCC | 0 | 7835 | 7851 | 3-10-4 | 665 |
| 599021 | 2601 | 2617 | Exon 18 | GCTGTTTTAATTCAATC | 33 | 7836 | 7852 | 3-10-4 | 666 |
| 599022 | 2602 | 2618 | Exon 18 | AGCTGTTTTAATTCAAT | 17 | 7837 | 7853 | 3-10-4 | 667 |
| 599023 | 2603 | 2619 | Exon 18 | CAGCTGTTTTAATTCAA | 52 | 7838 | 7854 | 3-10-4 | 668 |
| 532917 | 2604 | 2623 | Exon 18 | GTCGCAGCTGTTTTAATTCA | 86 | 7839 | 7858 | 5-10-5 | 317 |
| 599024 | 2604 | 2620 | Exon 18 | GCAGCTGTTTTAATTCA | 88 | 7839 | 7855 | 3-10-4 | 669 |
| 599025 | 2605 | 2621 | Exon 18 | CGCAGCTGTTTTAATTC | 85 | 7840 | 7856 | 3-10-4 | 670 |
| 599026 | 2606 | 2622 | Exon 18 | TCGCAGCTGTTTTAATT | 69 | 7841 | 7857 | 3-10-4 | 671 |
| 599027 | 2607 | 2623 | Exon 18 | GTCGCAGCTGTTTTAAT | 77 | 7842 | 7858 | 3-10-4 | 672 |
| 599028 | 2608 | 2624 | Exon 18 | TGTCGCAGCTGTTTTAA | 73 | 7843 | 7859 | 3-10-4 | 673 |
| 599029 | 2609 | 2625 | Exon 18 | TTGTCGCAGCTGTTTTA | 78 | 7844 | 7860 | 3-10-4 | 674 |
| 599030 | 2610 | 2626 | Exon 18 | GTTGTCGCAGCTGTTTT | 75 | 7845 | 7861 | 3-10-4 | 675 |
| 599031 | 2611 | 2627 | Exon 18 | TGTTGTCGCAGCTGTTT | 77 | 7846 | 7862 | 3-10-4 | 676 |
| 599032 | 2612 | 2628 | Exon 18/ | TTGTTGTCGCAGCTGTT | 79 | n/a | n/a | 3-10-4 | 677 |
| | | | Repeat | | | | | | |
| 599033 | 2613 | 2629 | Exon 18/ | TTTGTTGTCGCAGCTGT | 80 | n/a | n/a | 3-10-4 | 678 |
| | | | Repeat | | | | | | |
| 599034 | 2614 | 2630 | Exon 18/ | TTTTGTTGTCGCAGCTG | 78 | n/a | n/a | 3-10-4 | 679 |
| | | | Repeat | | | | | | |
| 599035 | 2615 | 2631 | Exon 18/ | TTTTTGTTGTCGCAGCT | 63 | n/a | n/a | 3-10-4 | 680 |
| | | | Repeat | | | | | | |

TABLE 135
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | Start | stop | Target | | inhi- | Start | Stop | | ID |
| NO | site | site | region | Sequence | bition | site | site | Motif | NO: |
| 599098 | 2552 | 2568 | Exon 18 | GAAAACCCAAATCCTCA | 57 | 7787 | 7803 | 4-8-5 | 619 |
| 599099 | 2553 | 2569 | Exon 18 | AGAAAACCCAAATCCTC | 33 | 7788 | 7804 | 4-8-5 | 620 |
| 599100 | 2554 | 2570 | Exon 18 | TAGAAAACCCAAATCCT | 32 | 7789 | 7805 | 4-8-5 | 621 |
| 599101 | 2555 | 2571 | Exon 18 | ATAGAAAACCCAAATCC | 47 | 7790 | 7806 | 4-8-5 | 622 |
| 599102 | 2557 | 2573 | Exon 18 | TTATAGAAAACCCAAAT | 59 | 7792 | 7808 | 4-8-5 | 623 |
| 599103 | 2558 | 2574 | Exon 18 | CTTATAGAAAACCCAAA | 10 | 7793 | 7809 | 4-8-5 | 624 |
| 599104 | 2559 | 2575 | Exon 18 | CCTTATAGAAAACCCAA | 3 | 7794 | 7810 | 4-8-5 | 625 |
| 599105 | 2560 | 2576 | Exon 18 | CCCTTATAGAAAACCCA | 45 | 7795 | 7811 | 4-8-5 | 626 |
| 599106 | 2561 | 2577 | Exon 18 | CCCCTTATAGAAAACCC | 49 | 7796 | 7812 | 4-8-5 | 627 |
| 599107 | 2562 | 2578 | Exon 18 | ACCCCTTATAGAAAACC | 35 | 7797 | 7813 | 4-8-5 | 628 |
| 599108 | 2563 | 2579 | Exon 18 | AACCCCTTATAGAAAAC | 17 | 7798 | 7814 | 4-8-5 | 629 |
| 599109 | 2564 | 2580 | Exon 18 | AAACCCCTTATAGAAAA | 36 | 7799 | 7815 | 4-8-5 | 630 |
| 599110 | 2565 | 2581 | Exon 18 | GAAACCCCTTATAGAAA | 20 | 7800 | 7816 | 4-8-5 | 631 |
| 599111 | 2566 | 2582 | Exon 18 | GGAAACCCCTTATAGAA | 20 | 7801 | 7817 | 4-8-5 | 632 |
| 599112 | 2567 | 2583 | Exon 18 | AGGAAACCCCTTATAGA | 15 | 7802 | 7818 | 4-8-5 | 633 |
| 599113 | 2568 | 2584 | Exon 18 | CAGGAAACCCCTTATAG | 19 | 7803 | 7819 | 4-8-5 | 634 |
| 599051 | 2568 | 2584 | Exon 18 | CAGGAAACCCCTTATAG | 26 | 7803 | 7819 | 5-7-5 | 634 |
| 599114 | 2569 | 2585 | Exon 18 | GCAGGAAACCCCTTATA | 18 | 7804 | 7820 | 4-8-5 | 635 |
| 599052 | 2569 | 2585 | Exon 18 | GCAGGAAACCCCTTATA | 21 | 7804 | 7820 | 5-7-5 | 635 |
| 599115 | 2570 | 2586 | Exon 18 | AGCAGGAAACCCCTTAT | 31 | 7805 | 7821 | 4-8-5 | 636 |
| 599053 | 2570 | 2586 | Exon 18 | AGCAGGAAACCCCTTAT | 25 | 7805 | 7821 | 5-7-5 | 636 |
| 599116 | 2571 | 2587 | Exon 18 | CAGCAGGAAACCCCTTA | 39 | 7806 | 7822 | 4-8-5 | 637 |
| 599054 | 2571 | 2587 | Exon 18 | CAGCAGGAAACCCCTTA | 36 | 7806 | 7822 | 5-7-5 | 637 |
| 599117 | 2572 | 2588 | Exon 18 | CCAGCAGGAAACCCCTT | 46 | 7807 | 7823 | 4-8-5 | 638 |
| 599055 | 2572 | 2588 | Exon 18 | CCAGCAGGAAACCCCTT | 22 | 7807 | 7823 | 5-7-5 | 638 |
| 599118 | 2573 | 2589 | Exon 18 | TCCAGCAGGAAACCCCT | 40 | 7808 | 7824 | 4-8-5 | 639 |
| 599056 | 2573 | 2589 | Exon 18 | TCCAGCAGGAAACCCCT | 32 | 7808 | 7824 | 5-7-5 | 639 |
| 599119 | 2574 | 2590 | Exon 18 | GTCCAGCAGGAAACCCC | 50 | 7809 | 7825 | 4-8-5 | 640 |
| 599057 | 2574 | 2590 | Exon 18 | GTCCAGCAGGAAACCCC | 46 | 7809 | 7825 | 5-7-5 | 640 |
| 599120 | 2575 | 2591 | Exon 18 | TGTCCAGCAGGAAACCC | 30 | 7810 | 7826 | 4-8-5 | 641 |
| 599058 | 2575 | 2591 | Exon 18 | TGTCCAGCAGGAAACCC | 52 | 7810 | 7826 | 5-7-5 | 641 |
| 599121 | 2576 | 2592 | Exon 18 | CTGTCCAGCAGGAAACC | 31 | 7811 | 7827 | 4-8-5 | 642 |
| 599059 | 2576 | 2592 | Exon 18 | CTGTCCAGCAGGAAACC | 24 | 7811 | 7827 | 5-7-5 | 642 |
| 599122 | 2577 | 2593 | Exon 18 | CCTGTCCAGCAGGAAAC | 23 | 7812 | 7828 | 4-8-5 | 643 |
| 599060 | 2577 | 2593 | Exon 18 | CCTGTCCAGCAGGAAAC | 37 | 7812 | 7828 | 5-7-5 | 643 |
| 599123 | 2578 | 2594 | Exon 18 | CCCTGTCCAGCAGGAAA | 51 | 7813 | 7829 | 4-8-5 | 644 |
| 599061 | 2578 | 2594 | Exon 18 | CCCTGTCCAGCAGGAAA | 34 | 7813 | 7829 | 5-7-5 | 644 |
| 599124 | 2580 | 2596 | Exon 18 | GCCCCTGTCCAGCAGGA | 56 | 7815 | 7831 | 4-8-5 | 645 |
| 599062 | 2580 | 2596 | Exon 18 | GCCCCTGTCCAGCAGGA | 51 | 7815 | 7831 | 5-7-5 | 645 |
| 599125 | 2581 | 2597 | Exon 18 | CGCCCCTGTCCAGCAGG | 70 | 7816 | 7832 | 4-8-5 | 646 |
| 599063 | 2581 | 2597 | Exon 18 | CGCCCCTGTCCAGCAGG | 56 | 7816 | 7832 | 5-7-5 | 646 |
| 599126 | 2582 | 2598 | Exon 18 | ACGCCCCTGTCCAGCAG | 76 | 7817 | 7833 | 4-8-5 | 647 |
| 599064 | 2582 | 2598 | Exon 18 | ACGCCCCTGTCCAGCAG | 61 | 7817 | 7833 | 5-7-5 | 647 |
| 599127 | 2583 | 2599 | Exon 18 | CACGCCCCTGTCCAGCA | 67 | 7818 | 7834 | 4-8-5 | 648 |
| 599065 | 2583 | 2599 | Exon 18 | CACGCCCCTGTCCAGCA | 64 | 7818 | 7834 | 5-7-5 | 648 |
| 599066 | 2584 | 2600 | Exon 18 | CCACGCCCCTGTCCAGC | 40 | 7819 | 7835 | 5-7-5 | 649 |
| 599067 | 2585 | 2601 | Exon 18 | CCCACGCCCCTGTCCAG | 37 | 7820 | 7836 | 5-7-5 | 650 |
| 599068 | 2586 | 2602 | Exon 18 | TCCCACGCCCCTGTCCA | 31 | 7821 | 7837 | 5-7-5 | 651 |
| 599069 | 2587 | 2603 | Exon 18 | ATCCCACGCCCCTGTCC | 39 | 7822 | 7838 | 5-7-5 | 652 |
| 599070 | 2588 | 2604 | Exon 18 | AATCCCACGCCCCTGTC | 59 | 7823 | 7839 | 5-7-5 | 653 |
| 599071 | 2589 | 2605 | Exon 18 | CAATCCCACGCCCCTGT | 63 | 7824 | 7840 | 5-7-5 | 657 |
| 599072 | 2590 | 2606 | Exon 18 | TCAATCCCACGCCCCTG | 74 | 7825 | 7841 | 5-7-5 | 655 |
| 599073 | 2591 | 2607 | Exon 18 | TTCAATCCCACGCCCCT | 53 | 7826 | 7842 | 5-7-5 | 656 |
| 599074 | 2592 | 2608 | Exon 18 | ATTCAATCCCACGCCCC | 56 | 7827 | 7843 | 5-7-5 | 657 |
| 599075 | 2593 | 2609 | Exon 18 | AATTCAATCCCACGCCC | 49 | 7828 | 7844 | 5-7-5 | 658 |
| 599076 | 2594 | 2610 | Exon 18 | TAATTCAATCCCACGCC | 54 | 7829 | 7845 | 5-7-5 | 659 |
| 599077 | 2595 | 2611 | Exon 18 | TTAATTCAATCCCACGC | 79 | 7830 | 7846 | 5-7-5 | 660 |
| 599078 | 2596 | 2612 | Exon 18 | TTTAATTCAATCCCACG | 67 | 7831 | 7847 | 5-7-5 | 661 |
| 599079 | 2597 | 2613 | Exon 18 | TTTTAATTCAATCCCAC | 69 | 7832 | 7848 | 5-7-5 | 662 |
| 599080 | 2598 | 2614 | Exon 18 | GTTTTAATTCAATCCCA | 79 | 7833 | 7849 | 5-7-5 | 663 |
| 599081 | 2599 | 2615 | Exon 18 | TGTTTTAATTCAATCCC | 57 | 7834 | 7850 | 5-7-5 | 664 |
| 599082 | 2600 | 2616 | Exon 18 | CTGTTTTAATTCAATCC | 50 | 7835 | 7851 | 5-7-5 | 665 |
| 599083 | 2601 | 2617 | Exon 18 | GCTGTTTTAATTCAATC | 67 | 7836 | 7852 | 5-7-5 | 666 |
| 599084 | 2602 | 2618 | Exon 18 | AGCTGTTTTAATTCAAT | 60 | 7837 | 7853 | 5-7-5 | 667 |
| 599085 | 2603 | 2619 | Exon 18 | CAGCTGTTTTAATTCAA | 71 | 7838 | 7854 | 5-7-5 | 668 |
| 532917 | 2604 | 2623 | Exon 18 | GTCGCAGCTGTTTTAATTCA | 82 | 7839 | 7858 | 5-10-5 | 317 |
| 599086 | 2604 | 2620 | Exon 18 | GCAGCTGTTTTAATTCA | 81 | 7839 | 7855 | 5-7-5 | 669 |
| 599087 | 2605 | 2621 | Exon 18 | CGCAGCTGTTTTAATTC | 88 | 7840 | 7856 | 5-7-5 | 670 |
| 599088 | 2606 | 2622 | Exon 18 | TCGCAGCTGTTTTAATT | 84 | 7841 | 7857 | 5-7-5 | 671 |
| 599089 | 2607 | 2623 | Exon 18 | GTCGCAGCTGTTTTAAT | 81 | 7842 | 7858 | 5-7-5 | 672 |
| 599090 | 2608 | 2624 | Exon 18 | TGTCGCAGCTGTTTTAA | 77 | 7843 | 7859 | 5-7-5 | 673 |
| 599091 | 2609 | 2625 | Exon 18 | TTGTCGCAGCTGTTTTA | 74 | 7844 | 7860 | 5-7-5 | 674 |
| 599092 | 2610 | 2626 | Exon 18 | GTTGTCGCAGCTGTTTT | 66 | 7845 | 7861 | 5-7-5 | 675 |
| 599093 | 2611 | 2627 | Exon 18 | TGTTGTCGCAGCTGTTT | 89 | 7846 | 7862 | 5-7-5 | 676 |
| 599094 | 2612 | 2628 | Exon 18/ | TTGTTGTCGCAGCTGTT | 82 | n/a | n/a | 5-7-5 | 677 |
| | | | Repeat | | | | | | |
| 599095 | 2613 | 2629 | Exon 18/ | TTTGTTGTCGCAGCTGT | 87 | n/a | n/a | 5-7-5 | 678 |
| | | | Repeat | | | | | | |
| 599096 | 2614 | 2630 | Exon 18/ | TTTTGTTGTCGCAGCTG | 85 | n/a | n/a | 5-7-5 | 679 |
| | | | Repeat | | | | | | |
| 599097 | 2615 | 2631 | Exon 18/ | TTTTTGTTGTCGCAGCT | 78 | n/a | n/a | 5-7-5 | 680 |
| | | | Repeat | | | | | | |

TABLE 136
Inhibition of CFB mRNA by MOE gapmers targeting SEQ ID NO: 1 or SEQ ID NO: 2

| | SEQ | | | | | SEQ | SEQ | | |
|---|---|---|---|---|---|---|---|---|---|
| | ID | SEQ ID | | | | ID | ID | | |
| | NO: 1 | NO: 1 | | | | NO: 2 | NO: 2 | | SEQ |
| ISIS | start | stop | Target | | % | start | stop | | ID |
| NO | site | site | region | Sequence | inhibition | site | site | Motif | NO: |
| 599510 | 2552 | 2570 | Exon 18 | TAGAAAACCCAAATCCTCA | 45 | 7787 | 7805 | 5-9-5 | 681 |
| 599331 | 2553 | 2571 | Exon 18 | ATAGAAAACCCAAATCCTC | 46 | 7788 | 7806 | 5-9-5 | 682 |
| 599332 | 2554 | 2572 | Exon 18 | TATAGAAAACCCAAATCCT | 38 | 7789 | 7807 | 5-9-5 | 683 |
| 599333 | 2556 | 2574 | Exon 18 | CTTATAGAAAACCCAAATC | 1 | 7791 | 7809 | 5-9-5 | 684 |
| 599334 | 2557 | 2575 | Exon 18 | CCTTATAGAAAACCCAAAT | 5 | 7792 | 7810 | 5-9-5 | 685 |
| 599335 | 2558 | 2576 | Exon 18 | CCCTTATAGAAAACCCAAA | 34 | 7793 | 7811 | 5-9-5 | 686 |
| 599336 | 2559 | 2577 | Exon 18 | CCCCTTATAGAAAACCCAA | 40 | 7794 | 7812 | 5-9-5 | 687 |
| 599337 | 2560 | 2578 | Exon 18 | ACCCCTTATAGAAAACCCA | 39 | 7795 | 7813 | 5-9-5 | 688 |
| 599338 | 2561 | 2579 | Exon 18 | AACCCCTTATAGAAAACCC | 57 | 7796 | 7814 | 5-9-5 | 689 |
| 599339 | 2562 | 2580 | Exon 18 | AAACCCCTTATAGAAAACC | 26 | 7797 | 7815 | 5-9-5 | 690 |
| 599281 | 2562 | 2580 | Exon 18 | AAACCCCTTATAGAAAACC | 15 | 7797 | 7815 | 6-7-6 | 690 |
| 599340 | 2563 | 2581 | Exon 18 | GAAACCCCTTATAGAAAAC | 17 | 7798 | 7816 | 5-9-5 | 691 |
| 599282 | 2563 | 2581 | Exon 18 | GAAACCCCTTATAGAAAAC | 12 | 7798 | 7816 | 6-7-6 | 691 |
| 599341 | 2564 | 2582 | Exon 18 | GGAAACCCCTTATAGAAAA | 23 | 7799 | 7817 | 5-9-5 | 692 |
| 599283 | 2564 | 2582 | Exon 18 | GGAAACCCCTTATAGAAAA | 18 | 7799 | 7817 | 6-7-6 | 692 |
| 599342 | 2565 | 2583 | Exon 18 | AGGAAACCCCTTATAGAAA | 10 | 7800 | 7818 | 5-9-5 | 693 |
| 599284 | 2565 | 2583 | Exon 18 | AGGAAACCCCTTATAGAAA | 14 | 7800 | 7818 | 6-7-6 | 693 |
| 599343 | 2566 | 2584 | Exon 18 | CAGGAAACCCCTTATAGAA | 10 | 7801 | 7819 | 5-9-5 | 694 |
| 599285 | 2566 | 2584 | Exon 18 | CAGGAAACCCCTTATAGAA | 13 | 7801 | 7819 | 6-7-6 | 694 |
| 599344 | 2567 | 2585 | Exon 18 | GCAGGAAACCCCTTATAGA | 22 | 7802 | 7820 | 5-9-5 | 695 |
| 599286 | 2567 | 2585 | Exon 18 | GCAGGAAACCCCTTATAGA | 31 | 7802 | 7820 | 6-7-6 | 695 |
| 599345 | 2568 | 2586 | Exon 18 | AGCAGGAAACCCCTTATAG | 19 | 7803 | 7821 | 5-9-5 | 696 |
| 599287 | 2568 | 2586 | Exon 18 | AGCAGGAAACCCCTTATAG | 12 | 7803 | 7821 | 6-7-6 | 696 |
| 599346 | 2569 | 2587 | Exon 18 | CAGCAGGAAACCCCTTATA | 30 | 7804 | 7822 | 5-9-5 | 697 |
| 599288 | 2569 | 2587 | Exon 18 | CAGCAGGAAACCCCTTATA | 28 | 7804 | 7822 | 6-7-6 | 697 |
| 599347 | 2570 | 2588 | Exon 18 | CCAGCAGGAAACCCCTTAT | 46 | 7805 | 7823 | 5-9-5 | 698 |
| 599289 | 2570 | 2588 | Exon 18 | CCAGCAGGAAACCCCTTAT | 32 | 7805 | 7823 | 6-7-6 | 698 |
| 599348 | 2571 | 2589 | Exon 18 | TCCAGCAGGAAACCCCTTA | 44 | 7806 | 7824 | 5-9-5 | 699 |
| 599290 | 2571 | 2589 | Exon 18 | TCCAGCAGGAAACCCCTTA | 24 | 7806 | 7824 | 6-7-6 | 699 |
| 599349 | 2572 | 2590 | Exon 18 | GTCCAGCAGGAAACCCCTT | 60 | 7807 | 7825 | 5-9-5 | 700 |
| 599291 | 2572 | 2590 | Exon 18 | GTCCAGCAGGAAACCCCTT | 38 | 7807 | 7825 | 6-7-6 | 700 |
| 599350 | 2573 | 2591 | Exon 18 | TGTCCAGCAGGAAACCCCT | 49 | 7808 | 7826 | 5-9-5 | 701 |
| 599292 | 2573 | 2591 | Exon 18 | TGTCCAGCAGGAAACCCCT | 35 | 7808 | 7826 | 6-7-6 | 701 |
| 599351 | 2575 | 2593 | Exon 18 | CCTGTCCAGCAGGAAACCC | 46 | 7810 | 7828 | 5-9-5 | 702 |
| 599293 | 2575 | 2593 | Exon 18 | CCTGTCCAGCAGGAAACCC | 12 | 7810 | 7828 | 6-7-6 | 702 |
| 599352 | 2576 | 2594 | Exon 18 | CCCTGTCCAGCAGGAAACC | 49 | 7811 | 7829 | 5-9-5 | 703 |
| 599294 | 2576 | 2594 | Exon 18 | CCCTGTCCAGCAGGAAACC | 38 | 7811 | 7829 | 6-7-6 | 703 |
| 599353 | 2577 | 2595 | Exon 18 | CCCCTGTCCAGCAGGAAAC | 64 | 7812 | 7830 | 5-9-5 | 704 |
| 599295 | 2577 | 2595 | Exon 18 | CCCCTGTCCAGCAGGAAAC | 33 | 7812 | 7830 | 6-7-6 | 704 |
| 599354 | 2578 | 2596 | Exon 18 | GCCCCTGTCCAGCAGGAAA | 56 | 7813 | 7831 | 5-9-5 | 705 |
| 599296 | 2578 | 2596 | Exon 18 | GCCCCTGTCCAGCAGGAAA | 13 | 7813 | 7831 | 6-7-6 | 705 |
| 599355 | 2580 | 2598 | Exon 18 | ACGCCCCTGTCCAGCAGGA | 81 | 7815 | 7833 | 5-9-5 | 706 |
| 599297 | 2580 | 2598 | Exon 18 | ACGCCCCTGTCCAGCAGGA | 57 | 7815 | 7833 | 6-7-6 | 706 |
| 599356 | 2581 | 2599 | Exon 18 | CACGCCCCTGTCCAGCAGG | 64 | 7816 | 7834 | 5-9-5 | 707 |
| 599298 | 2581 | 2599 | Exon 18 | CACGCCCCTGTCCAGCAGG | 39 | 7816 | 7834 | 6-7-6 | 707 |
| 599299 | 2582 | 2600 | Exon 18 | CCACGCCCCTGTCCAGCAG | 55 | 7817 | 7835 | 6-7-6 | 708 |
| 599300 | 2583 | 2601 | Exon 18 | CCCACGCCCCTGTCCAGCA | 45 | 7818 | 7836 | 6-7-6 | 709 |
| 599301 | 2584 | 2602 | Exon 18 | TCCCACGCCCCTGTCCAGC | 39 | 7819 | 7837 | 6-7-6 | 710 |
| 599302 | 2585 | 2603 | Exon 18 | ATCCCACGCCCCTGTCCAG | 27 | 7820 | 7838 | 6-7-6 | 711 |
| 599303 | 2586 | 2604 | Exon 18 | AATCCCACGCCCCTGTCCA | 35 | 7821 | 7839 | 6-7-6 | 712 |
| 599304 | 2587 | 2605 | Exon 18 | CAATCCCACGCCCCTGTCC | 16 | 7822 | 7840 | 6-7-6 | 713 |
| 599305 | 2588 | 2606 | Exon 18 | TCAATCCCACGCCCCTGTC | 41 | 7823 | 7841 | 6-7-6 | 714 |
| 599306 | 2589 | 2607 | Exon 18 | TTCAATCCCACGCCCCTGT | 70 | 7824 | 7842 | 6-7-6 | 715 |
| 599307 | 2590 | 2608 | Exon 18 | ATTCAATCCCACGCCCCTG | 66 | 7825 | 7843 | 6-7-6 | 716 |
| 599308 | 2591 | 2609 | Exon 18 | AATTCAATCCCACGCCCCT | 68 | 7826 | 7844 | 6-7-6 | 717 |
| 599309 | 2592 | 2610 | Exon 18 | TAATTCAATCCCACGCCCC | 52 | 7827 | 7845 | 6-7-6 | 718 |
| 599310 | 2593 | 2611 | Exon 18 | TTAATTCAATCCCACGCCC | 39 | 7828 | 7846 | 6-7-6 | 719 |
| 599311 | 2594 | 2612 | Exon 18 | TTTAATTCAATCCCACGCC | 83 | 7829 | 7847 | 6-7-6 | 720 |
| 599312 | 2595 | 2613 | Exon 18 | TTTTAATTCAATCCCACGC | 72 | 7830 | 7848 | 6-7-6 | 721 |
| 599313 | 2596 | 2614 | Exon 18 | GTTTTAATTCAATCCCACG | 86 | 7831 | 7849 | 6-7-6 | 722 |
| 599314 | 2597 | 2615 | Exon 18 | TGTTTTAATTCAATCCCAC | 91 | 7832 | 7850 | 6-7-6 | 723 |
| 599315 | 2598 | 2616 | Exon 18 | CTGTTTTAATTCAATCCCA | 71 | 7833 | 7851 | 6-7-6 | 724 |
| 599316 | 2599 | 2617 | Exon 18 | GCTGTTTTAATTCAATCCC | 89 | 7834 | 7852 | 6-7-6 | 725 |
| 599317 | 2600 | 2618 | Exon 18 | AGCTGTTTTAATTCAATCC | 87 | 7835 | 7853 | 6-7-6 | 726 |
| 599318 | 2601 | 2619 | Exon 18 | CAGCTGTTTTAATTCAATC | 81 | 7836 | 7854 | 6-7-6 | 727 |
| 599319 | 2602 | 2620 | Exon 18 | GCAGCTGTTTTAATTCAAT | 75 | 7837 | 7855 | 6-7-6 | 728 |
| 599320 | 2603 | 2621 | Exon 18 | CGCAGCTGTTTTAATTCAA | 84 | 7838 | 7856 | 6-7-6 | 729 |
| 532917 | 2604 | 2623 | Exon 18 | GTCGCAGCTGTTTTAATTCA | 92 | 7839 | 7858 | 5-10-5 | 317 |
| 599321 | 2604 | 2622 | Exon 18 | TCGCAGCTGTTTTAATTCA | 90 | 7839 | 7857 | 6-7-6 | 730 |
| 599322 | 2605 | 2623 | Exon 18 | GTCGCAGCTGTTTTAATTC | 89 | 7840 | 7858 | 6-7-6 | 731 |
| 599323 | 2606 | 2624 | Exon 18 | TGTCGCAGCTGTTTTAATT | 81 | 7841 | 7859 | 6-7-6 | 732 |
| 599324 | 2607 | 2625 | Exon 18 | TTGTCGCAGCTGTTTTAAT | 68 | 7842 | 7860 | 6-7-6 | 733 |
| 599325 | 2608 | 2626 | Exon 18 | GTTGTCGCAGCTGTTTTAA | 71 | 7843 | 7861 | 6-7-6 | 734 |
| 599326 | 2609 | 2627 | Exon 18 | TGTTGTCGCAGCTGTTTTA | 52 | 7844 | 7862 | 6-7-6 | 735 |
| 599327 | 2610 | 2628 | Exon 18/ | TTGTTGTCGCAGCTGTTTT | 88 | n/a | n/a | 6-7-6 | 736 |
| | | | Repeat | | | | | | |
| 599328 | 2611 | 2629 | Exon 18/ | TTTGTTGTCGCAGCTGTTT | 87 | n/a | n/a | 6-7-6 | 737 |
| | | | Repeat | | | | | | |
| 599329 | 2612 | 2630 | Exon 18/ | TTTTGTTGTCGCAGCTGTT | 84 | n/a | n/a | 6-7-6 | 738 |
| | | | Repeat | | | | | | |
| 599330 | 2613 | 2631 | Exon 18/ | TTTTTGTTGTCGCAGCTGT | 87 | n/a | n/a | 6-7-6 | 739 |
| | | | Repeat | | | | | | |

TABLE 137
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | Start | stop | Target | | inhi- | Start | Stop | | ID |
| NO | site | site | region | Sequence | bition | site | site | Motif | NO: |
| 599512 | 2552 | 2571 | Exon 18 | ATAGAAAACCCAAATCCTCA | 74 | 7787 | 7806 | 3-10-7 | 410 |
| 599449 | 2553 | 2572 | Exon 18 | TATAGAAAACCCAAATCCTC | 43 | 7788 | 7807 | 3-10-7 | 411 |
| 599450 | 2554 | 2573 | Exon 18 | TTATAGAAAACCCAAATCCT | 51 | 7789 | 7808 | 3-10-7 | 412 |
| 599451 | 2555 | 2574 | Exon 18 | CTTATAGAAAACCCAAATCC | 35 | 7790 | 7809 | 3-10-7 | 413 |
| 599452 | 2556 | 2575 | Exon 18 | CCTTATAGAAAACCCAAATC | 34 | 7791 | 7810 | 3-10-7 | 414 |
| 599453 | 2557 | 2576 | Exon 18 | CCCTTATAGAAAACCCAAAT | 44 | 7792 | 7811 | 3-10-7 | 415 |
| 599454 | 2558 | 2577 | Exon 18 | CCCCTTATAGAAAACCCAAA | 54 | 7793 | 7812 | 3-10-7 | 416 |
| 599455 | 2559 | 2578 | Exon 18 | ACCCCTTATAGAAAACCCAA | 53 | 7794 | 7813 | 3-10-7 | 417 |
| 599456 | 2560 | 2579 | Exon 18 | AACCCCTTATAGAAAACCCA | 69 | 7795 | 7814 | 3-10-7 | 418 |
| 599457 | 2561 | 2580 | Exon 18 | AAACCCCTTATAGAAAACCC | 46 | 7796 | 7815 | 3-10-7 | 419 |
| 599458 | 2562 | 2581 | Exon 18 | GAAACCCCTTATAGAAAACC | 0 | 7797 | 7816 | 3-10-7 | 420 |
| 599459 | 2563 | 2582 | Exon 18 | GGAAACCCCTTATAGAAAAC | 12 | 7798 | 7817 | 3-10-7 | 421 |
| 599460 | 2564 | 2583 | Exon 18 | AGGAAACCCCTTATAGAAAA | 17 | 7799 | 7818 | 3-10-7 | 422 |
| 599461 | 2565 | 2584 | Exon 18 | CAGGAAACCCCTTATAGAAA | 24 | 7800 | 7819 | 3-10-7 | 423 |
| 599462 | 2566 | 2585 | Exon 18 | GCAGGAAACCCCTTATAGAA | 33 | 7801 | 7820 | 3-10-7 | 424 |
| 599463 | 2567 | 2586 | Exon 18 | AGCAGGAAACCCCTTATAGA | 38 | 7802 | 7821 | 3-10-7 | 425 |
| 599464 | 2568 | 2587 | Exon 18 | CAGCAGGAAACCCCTTATAG | 33 | 7803 | 7822 | 3-10-7 | 426 |
| 599465 | 2569 | 2588 | Exon 18 | CCAGCAGGAAACCCCTTATA | 49 | 7804 | 7823 | 3-10-7 | 427 |
| 599466 | 2570 | 2589 | Exon 18 | TCCAGCAGGAAACCCCTTAT | 45 | 7805 | 7824 | 3-10-7 | 428 |
| 599467 | 2571 | 2590 | Exon 18 | GTCCAGCAGGAAACCCCTTA | 60 | 7806 | 7825 | 3-10-7 | 237 |
| 599468 | 2572 | 2591 | Exon 18 | TGTCCAGCAGGAAACCCCTT | 61 | 7807 | 7826 | 3-10-7 | 429 |
| 599469 | 2573 | 2592 | Exon 18 | CTGTCCAGCAGGAAACCCCT | 52 | 7808 | 7827 | 3-10-7 | 430 |
| 599470 | 2574 | 2593 | Exon 18 | CCTGTCCAGCAGGAAACCCC | 45 | 7809 | 7828 | 3-10-7 | 431 |
| 599471 | 2575 | 2594 | Exon 18 | CCCTGTCCAGCAGGAAACCC | 67 | 7810 | 7829 | 3-10-7 | 432 |
| 599472 | 2576 | 2595 | Exon 18 | CCCCTGTCCAGCAGGAAACC | 79 | 7811 | 7830 | 3-10-7 | 433 |
| 599473 | 2577 | 2596 | Exon 18 | GCCCCTGTCCAGCAGGAAAC | 72 | 7812 | 7831 | 3-10-7 | 238 |
| 599474 | 2578 | 2597 | Exon 18 | CGCCCCTGTCCAGCAGGAAA | 87 | 7813 | 7832 | 3-10-7 | 434 |
| 599475 | 2579 | 2598 | Exon 18 | ACGCCCCTGTCCAGCAGGAA | 76 | 7814 | 7833 | 3-10-7 | 435 |
| 599476 | 2580 | 2599 | Exon 18 | CACGCCCCTGTCCAGCAGGA | 81 | 7815 | 7834 | 3-10-7 | 436 |
| 599477 | 2581 | 2600 | Exon 18 | CCACGCCCCTGTCCAGCAGG | 83 | 7816 | 7835 | 3-10-7 | 437 |
| 599478 | 2582 | 2601 | Exon 18 | CCCACGCCCCTGTCCAGCAG | 72 | 7817 | 7836 | 3-10-7 | 438 |
| 599479 | 2583 | 2602 | Exon 18 | TCCCACGCCCCTGTCCAGCA | 81 | 7818 | 7837 | 3-10-7 | 439 |
| 599480 | 2584 | 2603 | Exon 18 | ATCCCACGCCCCTGTCCAGC | 77 | 7819 | 7838 | 3-10-7 | 440 |
| 599481 | 2585 | 2604 | Exon 18 | AATCCCACGCCCCTGTCCAG | 83 | 7820 | 7839 | 3-10-7 | 441 |
| 599482 | 2586 | 2605 | Exon 18 | CAATCCCACGCCCCTGTCCA | 87 | 7821 | 7840 | 3-10-7 | 442 |
| 599483 | 2587 | 2606 | Exon 18 | TCAATCCCACGCCCCTGTCC | 90 | 7822 | 7841 | 3-10-7 | 443 |
| 599484 | 2588 | 2607 | Exon 18 | TTCAATCCCACGCCCCTGTC | 72 | 7823 | 7842 | 3-10-7 | 444 |
| 599485 | 2589 | 2608 | Exon 18 | ATTCAATCCCACGCCCCTGT | 82 | 7824 | 7843 | 3-10-7 | 445 |
| 599486 | 2590 | 2609 | Exon 18 | AATTCAATCCCACGCCCCTG | 84 | 7825 | 7844 | 3-10-7 | 446 |
| 599487 | 2591 | 2610 | Exon 18 | TAATTCAATCCCACGCCCCT | 84 | 7826 | 7845 | 3-10-7 | 447 |
| 599488 | 2592 | 2611 | Exon 18 | TTAATTCAATCCCACGCCCC | 87 | 7827 | 7846 | 3-10-7 | 448 |
| 599489 | 2593 | 2612 | Exon 18 | TTTAATTCAATCCCACGCCC | 87 | 7828 | 7847 | 3-10-7 | 449 |
| 599490 | 2594 | 2613 | Exon 18 | TTTTAATTCAATCCCACGCC | 86 | 7829 | 7848 | 3-10-7 | 450 |
| 599491 | 2595 | 2614 | Exon 18 | GTTTTAATTCAATCCCACGC | 87 | 7830 | 7849 | 3-10-7 | 451 |
| 599492 | 2596 | 2615 | Exon 18 | TGTTTTAATTCAATCCCACG | 88 | 7831 | 7850 | 3-10-7 | 452 |
| 599493 | 2597 | 2616 | Exon 18 | CTGTTTTAATTCAATCCCAC | 75 | 7832 | 7851 | 3-10-7 | 453 |
| 599433 | 2597 | 2616 | Exon 18 | CTGTTTTAATTCAATCCCAC | 89 | 7832 | 7851 | 6-8-6 | 453 |
| 599494 | 2598 | 2617 | Exon 18 | GCTGTTTTAATTCAATCCCA | 90 | 7833 | 7852 | 3-10-7 | 454 |
| 599434 | 2598 | 2617 | Exon 18 | GCTGTTTTAATTCAATCCCA | 89 | 7833 | 7852 | 6-8-6 | 454 |
| 599495 | 2599 | 2618 | Exon 18 | AGCTGTTTTAATTCAATCCC | 88 | 7834 | 7853 | 3-10-7 | 239 |
| 599435 | 2599 | 2618 | Exon 18 | AGCTGTTTTAATTCAATCCC | 91 | 7834 | 7853 | 6-8-6 | 239 |
| 599496 | 2600 | 2619 | Exon 18 | CAGCTGTTTTAATTCAATCC | 89 | 7835 | 7854 | 3-10-7 | 455 |
| 599436 | 2600 | 2619 | Exon 18 | CAGCTGTTTTAATTCAATCC | 89 | 7835 | 7854 | 6-8-6 | 455 |
| 599497 | 2601 | 2620 | Exon 18 | GCAGCTGTTTTAATTCAATC | 89 | 7836 | 7855 | 3-10-7 | 456 |
| 599437 | 2601 | 2620 | Exon 18 | GCAGCTGTTTTAATTCAATC | 91 | 7836 | 7855 | 6-8-6 | 456 |
| 599498 | 2602 | 2621 | Exon 18 | CGCAGCTGTTTTAATTCAAT | 88 | 7837 | 7856 | 3-10-7 | 457 |
| 599438 | 2602 | 2621 | Exon 18 | CGCAGCTGTTTTAATTCAAT | 90 | 7837 | 7856 | 6-8-6 | 457 |
| 599499 | 2603 | 2622 | Exon 18 | TCGCAGCTGTTTTAATTCAA | 81 | 7838 | 7857 | 3-10-7 | 458 |
| 599439 | 2603 | 2622 | Exon 18 | TCGCAGCTGTTTTAATTCAA | 88 | 7838 | 7857 | 6-8-6 | 458 |
| 532917 | 2604 | 2623 | Exon 18 | GTCGCAGCTGTTTTAATTCA | 90 | 7839 | 7858 | 5-10-5 | 317 |
| 599500 | 2604 | 2623 | Exon 18 | GTCGCAGCTGTTTTAATTCA | 88 | 7839 | 7858 | 3-10-7 | 317 |
| 599440 | 2604 | 2623 | Exon 18 | GTCGCAGCTGTTTTAATTCA | 88 | 7839 | 7858 | 6-8-6 | 317 |
| 599501 | 2605 | 2624 | Exon 18 | TGTCGCAGCTGTTTTAATTC | 78 | 7840 | 7859 | 3-10-7 | 459 |
| 599441 | 2605 | 2624 | Exon 18 | TGTCGCAGCTGTTTTAATTC | 90 | 7840 | 7859 | 6-8-6 | 459 |
| 599502 | 2606 | 2625 | Exon 18 | TTGTCGCAGCTGTTTTAATT | 87 | 7841 | 7860 | 3-10-7 | 460 |
| 599442 | 2606 | 2625 | Exon 18 | TTGTCGCAGCTGTTTTAATT | 76 | 7841 | 7860 | 6-8-6 | 460 |
| 599503 | 2607 | 2626 | Exon 18 | GTTGTCGCAGCTGTTTTAAT | 83 | 7842 | 7861 | 3-10-7 | 461 |
| 599443 | 2607 | 2626 | Exon 18 | GTTGTCGCAGCTGTTTTAAT | 77 | 7842 | 7861 | 6-8-6 | 461 |
| 599504 | 2608 | 2627 | Exon 18 | TGTTGTCGCAGCTGTTTTAA | 89 | 7843 | 7862 | 3-10-7 | 395 |
| 599444 | 2608 | 2627 | Exon 18 | TGTTGTCGCAGCTGTTTTAA | 69 | 7843 | 7862 | 6-8-6 | 395 |
| 599505 | 2609 | 2628 | Exon 19/ | TTGTTGTCGCAGCTGTTTTA | 83 | n/a | n/a | 3-10-7 | 462 |
| | | | Repeat | | | | | | |
| 599445 | 2609 | 2628 | Exon 19/ | TTGTTGTCGCAGCTGTTTTA | 85 | n/a | n/a | 6-8-6 | 462 |
| | | | Repeat | | | | | | |
| 599506 | 2610 | 2629 | Exon 19/ | TTTGTTGTCGCAGCTGTTTT | 89 | n/a | n/a | 3-10-7 | 463 |
| | | | Repeat | | | | | | |
| 599446 | 2610 | 2629 | Exon 19/ | TTTGTTGTCGCAGCTGTTTT | 85 | n/a | n/a | 6-8-6 | 463 |
| | | | Repeat | | | | | | |
| 599507 | 2611 | 2630 | Exon 19/ | TTTTGTTGTCGCAGCTGTTT | 82 | n/a | n/a | 3-10-7 | 464 |
| | | | Repeat | | | | | | |
| 599447 | 2611 | 2630 | Exon 19/ | TTTTGTTGTCGCAGCTGTTT | 83 | n/a | n/a | 6-8-6 | 464 |
| | | | Repeat | | | | | | |
| 599508 | 2612 | 2631 | Exon 19/ | TTTTTGTTGTCGCAGCTGTT | 90 | n/a | n/a | 3-10-7 | 465 |
| | | | Repeat | | | | | | |
| 599448 | 2612 | 2631 | Exon 19/ | TTTTTGTTGTCGCAGCTGTT | 87 | n/a | n/a | 6-8-6 | 465 |
| | | | Repeat | | | | | | |

### Example 119: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 2,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as 4-8-5 MOE, 5-8-5 MOE, 5-9-5 MOE, 5-10-5 MOE, 6-7-6 MOE, 3-10-5 MOE, or 6-8-6 MOE gapmers.

The 4-8-5 MOE gapmers are 17 nucleosides in length, wherein the central gap segment comprises of eight 2′-deoxynucleosides and is flanked by wing segments on the 5′ direction and the 3′ direction comprising four and five nucleosides respectively. The 5-8-5 MOE gapmers are 18 nucleosides in length, wherein the central gap segment comprises of eight 2′-deoxynucleosides and is flanked by wing segments on the 5′ direction and the 3′ direction comprising five nucleosides each. The 5-9-5 MOE gapmers are 19 nucleosides in length, wherein the central gap segment comprises of nine 2′-deoxynucleosides and is flanked by wing segments on the 5′ direction and the 3′ direction comprising five nucleosides each. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. The 3-10-5 MOE gapmers are 18 nucleosides in length, wherein the central gap segment comprises of ten 2′-deoxynucleosides and is flanked by wing segments on the 5′ direction and the 3′ direction comprising three and five nucleosides respectively. The 6-7-6 MOE gapmers are 19 nucleosides in length, wherein the central gap segment comprises of seven 2′-deoxynucleosides and is flanked by wing segments on the 5′ direction and the 3′ direction comprising six nucleosides each. The 6-8-6 MOE gapmers are 20 nucleosides in length, wherein the central gap segment comprises of eight 2′-deoxynucleosides and is flanked by wing segments on the 5′ direction and the 3′ direction comprising six nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 10000 complementarity.

TABLE 138
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | Start | stop | Target | | inhi- | Start | Stop | | ID |
| NO | site | site | region | Sequence | bition | site | site | Motif | NO: |
| 599160 | 2560 | 2577 | Exon 18 | CCCCTTATAGAAAACCCA | 26 | 7795 | 7812 | 5-8-5 | 740 |
| 599161 | 2561 | 2578 | Exon 18 | ACCCCTTATAGAAAACCC | 20 | 7796 | 7813 | 5-8-5 | 741 |
| 599162 | 2562 | 2579 | Exon 18 | AACCCCTTATAGAAAACC | 12 | 7797 | 7814 | 5-8-5 | 742 |
| 599163 | 2563 | 2580 | Exon 18 | AAACCCCTTATAGAAAAC | 11 | 7798 | 7815 | 5-8-5 | 743 |
| 599164 | 2564 | 2581 | Exon 18 | GAAACCCCTTATAGAAAA | 11 | 7799 | 7816 | 5-8-5 | 744 |
| 599165 | 2566 | 2583 | Exon 18 | AGGAAACCCCTTATAGAA | 0 | 7801 | 7818 | 5-8-5 | 745 |
| 599166 | 2567 | 2584 | Exon 18 | CAGGAAACCCCTTATAGA | 12 | 7802 | 7819 | 5-8-5 | 746 |
| 599167 | 2568 | 2585 | Exon 18 | GCAGGAAACCCCTTATAG | 14 | 7803 | 7820 | 5-8-5 | 747 |
| 599168 | 2569 | 2586 | Exon 18 | AGCAGGAAACCCCTTATA | 16 | 7804 | 7821 | 5-8-5 | 748 |
| 599169 | 2570 | 2587 | Exon 18 | CAGCAGGAAACCCCTTAT | 24 | 7805 | 7822 | 5-8-5 | 749 |
| 599170 | 2571 | 2588 | Exon 18 | CCAGCAGGAAACCCCTTA | 37 | 7806 | 7823 | 5-8-5 | 750 |
| 599171 | 2572 | 2589 | Exon 18 | TCCAGCAGGAAACCCCTT | 30 | 7807 | 7824 | 5-8-5 | 751 |
| 599172 | 2573 | 2590 | Exon 18 | GTCCAGCAGGAAACCCCT | 43 | 7808 | 7825 | 5-8-5 | 752 |
| 599173 | 2574 | 2591 | Exon 18 | TGTCCAGCAGGAAACCCC | 47 | 7809 | 7826 | 5-8-5 | 753 |
| 599174 | 2575 | 2592 | Exon 18 | CTGTCCAGCAGGAAACCC | 27 | 7810 | 7827 | 5-8-5 | 754 |
| 599175 | 2576 | 2593 | Exon 18 | CCTGTCCAGCAGGAAACC | 30 | 7811 | 7828 | 5-8-5 | 755 |
| 599176 | 2577 | 2594 | Exon 18 | CCCTGTCCAGCAGGAAAC | 34 | 7812 | 7829 | 5-8-5 | 756 |
| 599177 | 2578 | 2595 | Exon 18 | CCCCTGTCCAGCAGGAAA | 41 | 7813 | 7830 | 5-8-5 | 757 |
| 599178 | 2580 | 2597 | Exon 18 | CGCCCCTGTCCAGCAGGA | 67 | 7815 | 7832 | 5-8-5 | 758 |
| 599179 | 2581 | 2598 | Exon 18 | ACGCCCCTGTCCAGCAGG | 61 | 7816 | 7833 | 5-8-5 | 759 |
| 599180 | 2582 | 2599 | Exon 18 | CACGCCCCTGTCCAGCAG | 62 | 7817 | 7834 | 5-8-5 | 760 |
| 599181 | 2583 | 2600 | Exon 18 | CCACGCCCCTGTCCAGCA | 63 | 7818 | 7835 | 5-8-5 | 761 |
| 599128 | 2584 | 2600 | Exon 18 | CCACGCCCCTGTCCAGC | 55 | 7819 | 7835 | 4-8-5 | 649 |
| 599182 | 2584 | 2601 | Exon 18 | CCCACGCCCCTGTCCAGC | 58 | 7819 | 7836 | 5-8-5 | 762 |
| 599129 | 2585 | 2601 | Exon 18 | CCCACGCCCCTGTCCAG | 41 | 7820 | 7836 | 4-8-5 | 650 |
| 599183 | 2585 | 2602 | Exon 18 | TCCCACGCCCCTGTCCAG | 43 | 7820 | 7837 | 5-8-5 | 763 |
| 599130 | 2586 | 2602 | Exon 18 | TCCCACGCCCCTGTCCA | 46 | 7821 | 7837 | 4-8-5 | 651 |
| 599184 | 2586 | 2603 | Exon 18 | ATCCCACGCCCCTGTCCA | 32 | 7821 | 7838 | 5-8-5 | 764 |
| 599131 | 2587 | 2603 | Exon 18 | ATCCCACGCCCCTGTCC | 30 | 7822 | 7838 | 4-8-5 | 652 |
| 599185 | 2587 | 2604 | Exon 18 | AATCCCACGCCCCTGTCC | 35 | 7822 | 7839 | 5-8-5 | 765 |
| 599132 | 2588 | 2604 | Exon 18 | AATCCCACGCCCCTGTC | 52 | 7823 | 7839 | 4-8-5 | 653 |
| 599186 | 2588 | 2605 | Exon 18 | CAATCCCACGCCCCTGTC | 55 | 7823 | 7840 | 5-8-5 | 766 |
| 599133 | 2589 | 2605 | Exon 18 | CAATCCCACGCCCCTGT | 66 | 7824 | 7840 | 4-8-5 | 654 |
| 599187 | 2589 | 2606 | Exon 18 | TCAATCCCACGCCCCTGT | 72 | 7824 | 7841 | 5-8-5 | 767 |
| 599134 | 2590 | 2606 | Exon 18 | TCAATCCCACGCCCCTG | 80 | 7825 | 7841 | 4-8-5 | 655 |
| 599188 | 2590 | 2607 | Exon 18 | TTCAATCCCACGCCCCTG | 92 | 7825 | 7842 | 5-8-5 | 768 |
| 599135 | 2591 | 2607 | Exon 18 | TTCAATCCCACGCCCCT | 61 | 7826 | 7842 | 4-8-5 | 656 |
| 599189 | 2591 | 2608 | Exon 18 | ATTCAATCCCACGCCCCT | 52 | 7826 | 7843 | 5-8-5 | 769 |
| 599136 | 2592 | 2608 | Exon 18 | ATTCAATCCCACGCCCC | 68 | 7827 | 7843 | 4-8-5 | 657 |
| 599190 | 2592 | 2609 | Exon 18 | AATTCAATCCCACGCCCC | 62 | 7827 | 7844 | 5-8-5 | 770 |
| 599137 | 2593 | 2609 | Exon 18 | AATTCAATCCCACGCCC | 51 | 7828 | 7844 | 4-8-5 | 658 |
| 599191 | 2593 | 2610 | Exon 18 | TAATTCAATCCCACGCCC | 54 | 7828 | 7845 | 5-8-5 | 771 |
| 599138 | 2594 | 2610 | Exon 18 | TAATTCAATCCCACGCC | 71 | 7829 | 7845 | 4-8-5 | 659 |
| 599192 | 2594 | 2611 | Exon 18 | TTAATTCAATCCCACGCC | 66 | 7829 | 7846 | 5-8-5 | 772 |
| 599139 | 2595 | 2611 | Exon 18 | TTAATTCAATCCCACGC | 80 | 7830 | 7846 | 4-8-5 | 660 |
| 599193 | 2595 | 2612 | Exon 18 | TTTAATTCAATCCCACGC | 74 | 7830 | 7847 | 5-8-5 | 773 |
| 599140 | 2596 | 2612 | Exon 18 | TTTAATTCAATCCCACG | 66 | 7831 | 7847 | 4-8-5 | 786 |
| 599194 | 2596 | 2613 | Exon 18 | TTTTAATTCAATCCCACG | 66 | 7831 | 7848 | 5-8-5 | 774 |
| 599141 | 2597 | 2613 | Exon 18 | TTTTAATTCAATCCCAC | 63 | 7832 | 7848 | 4-8-5 | 662 |
| 599195 | 2597 | 2614 | Exon 18 | GTTTTAATTCAATCCCAC | 86 | 7832 | 7849 | 5-8-5 | 775 |
| 599142 | 2598 | 2614 | Exon 18 | GTTTTAATTCAATCCCA | 69 | 7833 | 7849 | 4-8-5 | 663 |
| 599196 | 2598 | 2615 | Exon 18 | TGTTTTAATTCAATCCCA | 82 | 7833 | 7850 | 5-8-5 | 776 |
| 599143 | 2599 | 2615 | Exon 18 | TGTTTTAATTCAATCCC | 59 | 7834 | 7850 | 4-8-5 | 664 |
| 599197 | 2599 | 2616 | Exon 18 | CTGTTTTAATTCAATCCC | 79 | 7834 | 7851 | 5-8-5 | 777 |
| 599144 | 2600 | 2616 | Exon 18 | CTGTTTTAATTCAATCC | 52 | 7835 | 7851 | 4-8-5 | 665 |
| 599198 | 2600 | 2617 | Exon 18 | GCTGTTTTAATTCAATCC | 86 | 7835 | 7852 | 5-8-5 | 778 |
| 599145 | 2601 | 2617 | Exon 18 | GCTGTTTTAATTCAATC | 53 | 7836 | 7852 | 4-8-5 | 666 |
| 599199 | 2601 | 2618 | Exon 18 | AGCTGTTTTAATTCAATC | 72 | 7836 | 7853 | 5-8-5 | 779 |
| 599146 | 2602 | 2618 | Exon 18 | AGCTGTTTTAATTCAAT | 42 | 7837 | 7853 | 4-8-5 | 667 |
| 599200 | 2602 | 2619 | Exon 18 | CAGCTGTTTTAATTCAAT | 76 | 7837 | 7854 | 5-8-5 | 780 |
| 599147 | 2603 | 2619 | Exon 18 | CAGCTGTTTTAATTCAA | 55 | 7838 | 7854 | 4-8-5 | 668 |
| 599201 | 2603 | 2620 | Exon 18 | GCAGCTGTTTTAATTCAA | 87 | 7838 | 7855 | 5-8-5 | 781 |
| 532917 | 2604 | 2623 | Exon 18 | GTCGCAGCTGTTTTAATTCA | 93 | 7839 | 7858 | 5-10-5 | 317 |
| 599148 | 2604 | 2620 | Exon 18 | GCAGCTGTTTTAATTCA | 84 | 7839 | 7855 | 4-8-5 | 669 |
| 599202 | 2604 | 2621 | Exon 18 | CGCAGCTGTTTTAATTCA | 89 | 7839 | 7856 | 5-8-5 | 782 |
| 599149 | 2605 | 2621 | Exon 18 | CGCAGCTGTTTTAATTC | 92 | 7840 | 7856 | 4-8-5 | 670 |
| 599203 | 2605 | 2622 | Exon 18 | TCGCAGCTGTTTTAATTC | 90 | 7840 | 7857 | 5-8-5 | 783 |
| 599150 | 2606 | 2622 | Exon 18 | TCGCAGCTGTTTTAATT | 75 | 7841 | 7857 | 4-8-5 | 671 |
| 599151 | 2607 | 2623 | Exon 18 | GTCGCAGCTGTTTTAAT | 80 | 7842 | 7858 | 4-8-5 | 672 |
| 599152 | 2608 | 2624 | Exon 18 | TGTCGCAGCTGTTTTAA | 76 | 7843 | 7859 | 4-8-5 | 673 |
| 599153 | 2609 | 2625 | Exon 18 | TTGTCGCAGCTGTTTTA | 56 | 7844 | 7860 | 4-8-5 | 674 |
| 599154 | 2610 | 2626 | Exon 18 | GTTGTCGCAGCTGTTTT | 85 | 7845 | 7861 | 4-8-5 | 675 |
| 599155 | 2611 | 2627 | Exon 18 | TGTTGTCGCAGCTGTTT | 89 | 7846 | 7862 | 4-8-5 | 676 |
| 599156 | 2612 | 2628 | Exon 18/ | TTGTTGTCGCAGCTGTT | 83 | n/a | n/a | 4-8-5 | 813 |
| | | | Repeat | | | | | | |
| 599157 | 2613 | 2629 | Exon 18/ | TTTGTTGTCGCAGCTGT | 78 | n/a | n/a | 4-8-5 | 678 |
| | | | Repeat | | | | | | |
| 599158 | 2614 | 2630 | Exon 18/ | TTTTGTTGTCGCAGCTG | 83 | n/a | n/a | 4-8-5 | 679 |
| | | | Repeat | | | | | | |
| 599159 | 2615 | 2631 | Exon 18/ | TTTTTGTTGTCGCAGCT | 65 | n/a | n/a | 4-8-5 | 680 |
| | | | Repeat | | | | | | |
| 599204 | 2606 | 2623 | Exon 18 | GTCGCAGCTGTTTTAATT | 83 | 7841 | 7858 | 5-8-5 | 784 |

TABLE 139
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | Start | stop | Target | | inhi- | Start | Stop | | ID |
| NO | site | site | region | Sequence | bition | site | site | Motif | NO: |
| 599509 | 2552 | 2570 | Exon 18 | TAGAAAACCCAAATCCTCA | 45 | 7787 | 7805 | 6-7-6 | 681 |
| 599213 | 2553 | 2570 | Exon 18 | TAGAAAACCCAAATCCTC | 89 | 7788 | 7805 | 3-10-5 | 785 |
| 599273 | 2553 | 2571 | Exon 18 | ATAGAAAACCCAAATCCTC | 85 | 7788 | 7806 | 6-7-6 | 682 |
| 599214 | 2554 | 2571 | Exon 18 | ATAGAAAACCCAAATCCT | 79 | 7789 | 7806 | 3-10-5 | 786 |
| 599274 | 2554 | 2572 | Exon 18 | TATAGAAAACCCAAATCCT | 75 | 7789 | 7807 | 6-7-6 | 683 |
| 599215 | 2555 | 2572 | Exon 18 | TATAGAAAACCCAAATCC | 81 | 7790 | 7807 | 3-10-5 | 787 |
| 599216 | 2556 | 2573 | Exon 18 | TTATAGAAAACCCAAATC | 87 | 7791 | 7808 | 3-10-5 | 788 |
| 599275 | 2556 | 2574 | Exon 18 | CTTATAGAAAACCCAAATC | 84 | 7791 | 7809 | 6-7-6 | 684 |
| 599217 | 2557 | 2574 | Exon 18 | CTTATAGAAAACCCAAAT | 84 | 7792 | 7809 | 3-10-5 | 789 |
| 599276 | 2557 | 2575 | Exon 18 | CCTTATAGAAAACCCAAAT | 68 | 7792 | 7810 | 6-7-6 | 685 |
| 599218 | 2558 | 2575 | Exon 18 | CCTTATAGAAAACCCAAA | 82 | 7793 | 7810 | 3-10-5 | 790 |
| 599277 | 2558 | 2576 | Exon 18 | CCCTTATAGAAAACCCAAA | 82 | 7793 | 7811 | 6-7-6 | 686 |
| 599219 | 2559 | 2576 | Exon 18 | CCCTTATAGAAAACCCAA | 81 | 7794 | 7811 | 3-10-5 | 791 |
| 599278 | 2559 | 2577 | Exon 18 | CCCCTTATAGAAAACCCAA | 84 | 7794 | 7812 | 6-7-6 | 687 |
| 599220 | 2560 | 2577 | Exon 18 | CCCCTTATAGAAAACCCA | 92 | 7795 | 7812 | 3-10-5 | 740 |
| 599279 | 2560 | 2578 | Exon 18 | ACCCCTTATAGAAAACCCA | 92 | 7795 | 7813 | 6-7-6 | 688 |
| 599221 | 2561 | 2578 | Exon 18 | ACCCCTTATAGAAAACCC | 93 | 7796 | 7813 | 3-10-5 | 741 |
| 599280 | 2561 | 2579 | Exon 18 | AACCCCTTATAGAAAACCC | 90 | 7796 | 7814 | 6-7-6 | 689 |
| 599222 | 2562 | 2579 | Exon 18 | AACCCCTTATAGAAAACC | 95 | 7797 | 7814 | 3-10-5 | 742 |
| 599223 | 2563 | 2580 | Exon 18 | AAACCCCTTATAGAAAAC | 93 | 7798 | 7815 | 3-10-5 | 743 |
| 599224 | 2564 | 2581 | Exon 18 | GAAACCCCTTATAGAAAA | 90 | 7799 | 7816 | 3-10-5 | 744 |
| 599225 | 2566 | 2583 | Exon 18 | AGGAAACCCCTTATAGAA | 93 | 7801 | 7818 | 3-10-5 | 745 |
| 599226 | 2567 | 2584 | Exon 18 | CAGGAAACCCCTTATAGA | 95 | 7802 | 7819 | 3-10-5 | 746 |
| 599227 | 2568 | 2585 | Exon 18 | GCAGGAAACCCCTTATAG | 94 | 7803 | 7820 | 3-10-5 | 747 |
| 599228 | 2569 | 2586 | Exon 18 | AGCAGGAAACCCCTTATA | 96 | 7804 | 7821 | 3-10-5 | 748 |
| 599229 | 2570 | 2587 | Exon 18 | CAGCAGGAAACCCCTTAT | 92 | 7805 | 7822 | 3-10-5 | 749 |
| 599230 | 2571 | 2588 | Exon 18 | CCAGCAGGAAACCCCTTA | 88 | 7806 | 7823 | 3-10-5 | 750 |
| 599231 | 2572 | 2589 | Exon 18 | TCCAGCAGGAAACCCCTT | 83 | 7807 | 7824 | 3-10-5 | 751 |
| 599232 | 2573 | 2590 | Exon 18 | GTCCAGCAGGAAACCCCT | 89 | 7808 | 7825 | 3-10-5 | 752 |
| 599233 | 2574 | 2591 | Exon 18 | TGTCCAGCAGGAAACCCC | 83 | 7809 | 7826 | 3-10-5 | 753 |
| 599234 | 2575 | 2592 | Exon 18 | CTGTCCAGCAGGAAACCC | 88 | 7810 | 7827 | 3-10-5 | 754 |
| 599235 | 2576 | 2593 | Exon 18 | CCTGTCCAGCAGGAAACC | 91 | 7811 | 7828 | 3-10-5 | 755 |
| 599236 | 2577 | 2594 | Exon 18 | CCCTGTCCAGCAGGAAAC | 90 | 7812 | 7829 | 3-10-5 | 756 |
| 599237 | 2578 | 2595 | Exon 18 | CCCCTGTCCAGCAGGAAA | 34 | 7813 | 7830 | 3-10-5 | 757 |
| 599238 | 2580 | 2597 | Exon 18 | CGCCCCTGTCCAGCAGGA | 14 | 7815 | 7832 | 3-10-5 | 758 |
| 599239 | 2581 | 2598 | Exon 18 | ACGCCCCTGTCCAGCAGG | 10 | 7816 | 7833 | 3-10-5 | 759 |
| 599240 | 2582 | 2599 | Exon 18 | CACGCCCCTGTCCAGCAG | 26 | 7817 | 7834 | 3-10-5 | 760 |
| 599241 | 2583 | 2600 | Exon 18 | CCACGCCCCTGTCCAGCA | 11 | 7818 | 7835 | 3-10-5 | 761 |
| 599242 | 2584 | 2601 | Exon 18 | CCCACGCCCCTGTCCAGC | 24 | 7819 | 7836 | 3-10-5 | 762 |
| 599243 | 2585 | 2602 | Exon 18 | TCCCACGCCCCTGTCCAG | 23 | 7820 | 7837 | 3-10-5 | 763 |
| 599244 | 2586 | 2603 | Exon 18 | ATCCCACGCCCCTGTCCA | 29 | 7821 | 7838 | 3-10-5 | 764 |
| 599245 | 2587 | 2604 | Exon 18 | AATCCCACGCCCCTGTCC | 11 | 7822 | 7839 | 3-10-5 | 765 |
| 599246 | 2588 | 2605 | Exon 18 | CAATCCCACGCCCCTGTC | 0 | 7823 | 7840 | 3-10-5 | 766 |
| 599247 | 2589 | 2606 | Exon 18 | TCAATCCCACGCCCCTGT | 21 | 7824 | 7841 | 3-10-5 | 767 |
| 599248 | 2590 | 2607 | Exon 18 | TTCAATCCCACGCCCCTG | 0 | 7825 | 7842 | 3-10-5 | 768 |
| 599249 | 2591 | 2608 | Exon 18 | ATTCAATCCCACGCCCCT | 9 | 7826 | 7843 | 3-10-5 | 769 |
| 599250 | 2592 | 2609 | Exon 18 | AATTCAATCCCACGCCCC | 4 | 7827 | 7844 | 3-10-5 | 770 |
| 599251 | 2593 | 2610 | Exon 18 | TAATTCAATCCCACGCCC | 12 | 7828 | 7845 | 3-10-5 | 771 |
| 599252 | 2594 | 2611 | Exon 18 | TTAATTCAATCCCACGCC | 2 | 7829 | 7846 | 3-10-5 | 772 |
| 599253 | 2595 | 2612 | Exon 18 | TTTAATTCAATCCCACGC | 28 | 7830 | 7847 | 3-10-5 | 773 |
| 599254 | 2596 | 2613 | Exon 18 | TTTTAATTCAATCCCACG | 27 | 7831 | 7848 | 3-10-5 | 774 |
| 599255 | 2597 | 2614 | Exon 18 | GTTTTAATTCAATCCCAC | 38 | 7832 | 7849 | 3-10-5 | 775 |
| 599256 | 2598 | 2615 | Exon 18 | TGTTTTAATTCAATCCCA | 36 | 7833 | 7850 | 3-10-5 | 776 |
| 599257 | 2599 | 2616 | Exon 18 | CTGTTTTAATTCAATCCC | 48 | 7834 | 7851 | 3-10-5 | 777 |
| 599258 | 2600 | 2617 | Exon 18 | GCTGTTTTAATTCAATCC | 19 | 7835 | 7852 | 3-10-5 | 778 |
| 599259 | 2601 | 2618 | Exon 18 | AGCTGTTTTAATTCAATC | 36 | 7836 | 7853 | 3-10-5 | 779 |
| 599260 | 2602 | 2619 | Exon 18 | CAGCTGTTTTAATTCAAT | 58 | 7837 | 7854 | 3-10-5 | 780 |
| 599261 | 2603 | 2620 | Exon 18 | GCAGCTGTTTTAATTCAA | 35 | 7838 | 7855 | 3-10-5 | 781 |
| 532917 | 2604 | 2623 | Exon 18 | GTCGCAGCTGTTTTAATTCA | 96 | 7839 | 7858 | 5-10-5 | 317 |
| 599262 | 2604 | 2621 | Exon 18 | CGCAGCTGTTTTAATTCA | 52 | 7839 | 7856 | 3-10-5 | 782 |
| 599263 | 2605 | 2622 | Exon 18 | TCGCAGCTGTTTTAATTC | 66 | 7840 | 7857 | 3-10-5 | 783 |
| 599264 | 2606 | 2623 | Exon 18 | GTCGCAGCTGTTTTAATT | 48 | 7841 | 7858 | 3-10-5 | 784 |
| 599265 | 2607 | 2624 | Exon 18 | TGTCGCAGCTGTTTTAAT | 46 | 7842 | 7859 | 3-10-5 | 792 |
| 599205 | 2607 | 2624 | Exon 18 | TGTCGCAGCTGTTTTAAT | 83 | 7842 | 7859 | 5-8-5 | 792 |
| 599266 | 2608 | 2625 | Exon 18 | TTGTCGCAGCTGTTTTAA | 76 | 7843 | 7860 | 3-10-5 | 793 |
| 599206 | 2608 | 2625 | Exon 18 | TTGTCGCAGCTGTTTTAA | 90 | 7843 | 7860 | 5-8-5 | 793 |
| 599267 | 2609 | 2626 | Exon 18 | GTTGTCGCAGCTGTTTTA | 53 | 7844 | 7861 | 3-10-5 | 794 |
| 599207 | 2609 | 2626 | Exon 18 | GTTGTCGCAGCTGTTTTA | 82 | 7844 | 7861 | 5-8-5 | 794 |
| 599268 | 2610 | 2627 | Exon 18 | TGTTGTCGCAGCTGTTTT | 58 | 7845 | 7862 | 3-10-5 | 795 |
| 599208 | 2610 | 2627 | Exon 18 | TGTTGTCGCAGCTGTTTT | 70 | 7845 | 7862 | 5-8-5 | 795 |
| 599269 | 2611 | 2628 | Exon 18/ | TTGTTGTCGCAGCTGTTT | 38 | n/a | n/a | 3-10-5 | 796 |
| | | | Repeat | | | | | | |
| 599209 | 2611 | 2628 | Exon 18/ | TTGTTGTCGCAGCTGTTT | 50 | n/a | n/a | 5-8-5 | 796 |
| | | | Repeat | | | | | | |
| 599270 | 2612 | 2629 | Exon 18/ | TTTGTTGTCGCAGCTGTT | 46 | n/a | n/a | 3-10-5 | 797 |
| | | | Repeat | | | | | | |
| 599210 | 2612 | 2629 | Exon 18/ | TTTGTTGTCGCAGCTGTT | 76 | n/a | n/a | 5-8-5 | 797 |
| | | | Repeat | | | | | | |
| 599271 | 2613 | 2630 | Exon 18/ | TTTTGTTGTCGCAGCTGT | 64 | n/a | n/a | 3-10-5 | 798 |
| | | | Repeat | | | | | | |
| 599211 | 2613 | 2630 | Exon 18/ | TTTTGTTGTCGCAGCTGT | 78 | n/a | n/a | 5-8-5 | 798 |
| | | | Repeat | | | | | | |
| 599272 | 2614 | 2631 | Exon 18/ | TTTTTGTTGTCGCAGCTG | 89 | n/a | n/a | 3-10-5 | 799 |
| | | | Repeat | | | | | | |
| 599212 | 2614 | 2631 | Exon 18/ | TTTTTGTTGTCGCAGCTG | 84 | n/a | n/a | 5-8-5 | 799 |
| | | | Repeat | | | | | | |

TABLE 140
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | Start | stop | Target | | inhi- | Start | Stop | | ID |
| NO | site | site | region | Sequence | bition | site | site | Motif | NO: |
| 599511 | 2552 | 2571 | Exon 18 | ATAGAAAACCCAAATCCTCA | 38 | 7787 | 7806 | 6-8-6 | 410 |
| 599389 | 2553 | 2572 | Exon 18 | TATAGAAAACCCAAATCCTC | 80 | 7788 | 7807 | 6-8-6 | 411 |
| 599390 | 2554 | 2573 | Exon 18 | TTATAGAAAACCCAAATCCT | 92 | 7789 | 7808 | 6-8-6 | 412 |
| 599391 | 2555 | 2574 | Exon 18 | CTTATAGAAAACCCAAATCC | 90 | 7790 | 7809 | 6-8-6 | 413 |
| 599392 | 2556 | 2575 | Exon 18 | CCTTATAGAAAACCCAAATC | 87 | 7791 | 7810 | 6-8-6 | 414 |
| 599393 | 2557 | 2576 | Exon 18 | CCCTTATAGAAAACCCAAAT | 87 | 7792 | 7811 | 6-8-6 | 415 |
| 599394 | 2558 | 2577 | Exon 18 | CCCCTTATAGAAAACCCAAA | 74 | 7793 | 7812 | 6-8-6 | 416 |
| 599395 | 2559 | 2578 | Exon 18 | ACCCCTTATAGAAAACCCAA | 78 | 7794 | 7813 | 6-8-6 | 417 |
| 599396 | 2560 | 2579 | Exon 18 | AACCCCTTATAGAAAACCCA | 77 | 7795 | 7814 | 6-8-6 | 418 |
| 599397 | 2561 | 2580 | Exon 18 | AAACCCCTTATAGAAAACCC | 89 | 7796 | 7815 | 6-8-6 | 419 |
| 599398 | 2562 | 2581 | Exon 18 | GAAACCCCTTATAGAAAACC | 90 | 7797 | 7816 | 6-8-6 | 420 |
| 599399 | 2563 | 2582 | Exon 18 | GGAAACCCCTTATAGAAAAC | 91 | 7798 | 7817 | 6-8-6 | 421 |
| 599400 | 2564 | 2583 | Exon 18 | AGGAAACCCCTTATAGAAAA | 88 | 7799 | 7818 | 6-8-6 | 422 |
| 599401 | 2565 | 2584 | Exon 18 | CAGGAAACCCCTTATAGAAA | 85 | 7800 | 7819 | 6-8-6 | 423 |
| 599402 | 2566 | 2585 | Exon 18 | GCAGGAAACCCCTTATAGAA | 77 | 7801 | 7820 | 6-8-6 | 424 |
| 599403 | 2567 | 2586 | Exon 18 | AGCAGGAAACCCCTTATAGA | 85 | 7802 | 7821 | 6-8-6 | 425 |
| 599404 | 2568 | 2587 | Exon 18 | CAGCAGGAAACCCCTTATAG | 90 | 7803 | 7822 | 6-8-6 | 426 |
| 599405 | 2569 | 2588 | Exon 18 | CCAGCAGGAAACCCCTTATA | 89 | 7804 | 7823 | 6-8-6 | 427 |
| 599406 | 2570 | 2589 | Exon 18 | TCCAGCAGGAAACCCCTTAT | 72 | 7805 | 7824 | 6-8-6 | 428 |
| 599407 | 2571 | 2590 | Exon 18 | GTCCAGCAGGAAACCCCTTA | 87 | 7806 | 7825 | 6-8-6 | 237 |
| 599408 | 2572 | 2591 | Exon 18 | TGTCCAGCAGGAAACCCCTT | 87 | 7807 | 7826 | 6-8-6 | 429 |
| 599409 | 2573 | 2592 | Exon 18 | CTGTCCAGCAGGAAACCCCT | 83 | 7808 | 7827 | 6-8-6 | 430 |
| 599410 | 2574 | 2593 | Exon 18 | CCTGTCCAGCAGGAAACCCC | 88 | 7809 | 7828 | 6-8-6 | 431 |
| 599411 | 2575 | 2594 | Exon 18 | CCCTGTCCAGCAGGAAACCC | 45 | 7810 | 7829 | 6-8-6 | 432 |
| 599412 | 2576 | 2595 | Exon 18 | CCCCTGTCCAGCAGGAAACC | 66 | 7811 | 7830 | 6-8-6 | 433 |
| 599413 | 2577 | 2596 | Exon 18 | GCCCCTGTCCAGCAGGAAAC | 92 | 7812 | 7831 | 6-8-6 | 238 |
| 599414 | 2578 | 2597 | Exon 18 | CGCCCCTGTCCAGCAGGAAA | 92 | 7813 | 7832 | 6-8-6 | 434 |
| 599415 | 2579 | 2598 | Exon 18 | ACGCCCCTGTCCAGCAGGAA | 87 | 7814 | 7833 | 6-8-6 | 435 |
| 599416 | 2580 | 2599 | Exon 18 | CACGCCCCTGTCCAGCAGGA | 91 | 7815 | 7834 | 6-8-6 | 436 |
| 599417 | 2581 | 2600 | Exon 18 | CCACGCCCCTGTCCAGCAGG | 84 | 7816 | 7835 | 6-8-6 | 437 |
| 599357 | 2582 | 2600 | Exon 18 | CCACGCCCCTGTCCAGCAG | 88 | 7817 | 7835 | 5-9-5 | 708 |
| 599418 | 2582 | 2601 | Exon 18 | CCCACGCCCCTGTCCAGCAG | 85 | 7817 | 7836 | 6-8-6 | 438 |
| 599358 | 2583 | 2601 | Exon 18 | CCCACGCCCCTGTCCAGCA | 86 | 7818 | 7836 | 5-9-5 | 709 |
| 599419 | 2583 | 2602 | Exon 18 | TCCCACGCCCCTGTCCAGCA | 91 | 7818 | 7837 | 6-8-6 | 833 |
| 599359 | 2584 | 2602 | Exon 18 | TCCCACGCCCCTGTCCAGC | 85 | 7819 | 7837 | 5-9-5 | 834 |
| 599420 | 2584 | 2603 | Exon 18 | ATCCCACGCCCCTGTCCAGC | 91 | 7819 | 7838 | 6-8-6 | 440 |
| 599360 | 2585 | 2603 | Exon 18 | ATCCCACGCCCCTGTCCAG | 89 | 7820 | 7838 | 5-9-5 | 711 |
| 599421 | 2585 | 2604 | Exon 18 | AATCCCACGCCCCTGTCCAG | 87 | 7820 | 7839 | 6-8-6 | 441 |
| 599361 | 2586 | 2604 | Exon 18 | AATCCCACGCCCCTGTCCA | 89 | 7821 | 7839 | 5-9-5 | 712 |
| 599422 | 2586 | 2605 | Exon 18 | CAATCCCACGCCCCTGTCCA | 90 | 7821 | 7840 | 6-8-6 | 442 |
| 599362 | 2587 | 2605 | Exon 18 | CAATCCCACGCCCCTGTCC | 94 | 7822 | 7840 | 5-9-5 | 713 |
| 599423 | 2587 | 2606 | Exon 18 | TCAATCCCACGCCCCTGTCC | 85 | 7822 | 7841 | 6-8-6 | 841 |
| 599363 | 2588 | 2606 | Exon 18 | TCAATCCCACGCCCCTGTC | 88 | 7823 | 7841 | 5-9-5 | 714 |
| 599424 | 2588 | 2607 | Exon 18 | TTCAATCCCACGCCCCTGTC | 88 | 7823 | 7842 | 6-8-6 | 444 |
| 599364 | 2589 | 2607 | Exon 18 | TTCAATCCCACGCCCCTGT | 88 | 7824 | 7842 | 5-9-5 | 715 |
| 599425 | 2589 | 2608 | Exon 18 | ATTCAATCCCACGCCCCTGT | 68 | 7824 | 7843 | 6-8-6 | 445 |
| 599365 | 2590 | 2608 | Exon 18 | ATTCAATCCCACGCCCCTG | 48 | 7825 | 7843 | 5-9-5 | 716 |
| 599426 | 2590 | 2609 | Exon 18 | AATTCAATCCCACGCCCCTG | 55 | 7825 | 7844 | 6-8-6 | 446 |
| 599366 | 2591 | 2609 | Exon 18 | AATTCAATCCCACGCCCCT | 28 | 7826 | 7844 | 5-9-5 | 717 |
| 599427 | 2591 | 2610 | Exon 18 | TAATTCAATCCCACGCCCCT | 13 | 7826 | 7845 | 6-8-6 | 849 |
| 599367 | 2592 | 2610 | Exon 18 | TAATTCAATCCCACGCCCC | 21 | 7827 | 7845 | 5-9-5 | 718 |
| 599428 | 2592 | 2611 | Exon 18 | TTAATTCAATCCCACGCCCC | 39 | 7827 | 7846 | 6-8-6 | 448 |
| 599368 | 2593 | 2611 | Exon 18 | TTAATTCAATCCCACGCCC | 20 | 7828 | 7846 | 5-9-5 | 719 |
| 599429 | 2593 | 2612 | Exon 18 | TTTAATTCAATCCCACGCCC | 18 | 7828 | 7847 | 6-8-6 | 449 |
| 599369 | 2594 | 2612 | Exon 18 | TTTAATTCAATCCCACGCC | 78 | 7829 | 7847 | 5-9-5 | 720 |
| 599430 | 2594 | 2613 | Exon 18 | TTTTAATTCAATCCCACGCC | 24 | 7829 | 7848 | 6-8-6 | 450 |
| 599370 | 2595 | 2613 | Exon 18 | TTTTAATTCAATCCCACGC | 25 | 7830 | 7848 | 5-9-5 | 721 |
| 599431 | 2595 | 2614 | Exon 18 | GTTTTAATTCAATCCCACGC | 30 | 7830 | 7849 | 6-8-6 | 451 |
| 599371 | 2596 | 2614 | Exon 18 | GTTTTAATTCAATCCCACG | 84 | 7831 | 7849 | 5-9-5 | 722 |
| 599432 | 2596 | 2615 | Exon 18 | TGTTTTAATTCAATCCCACG | 29 | 7831 | 7850 | 6-8-6 | 452 |
| 599372 | 2597 | 2615 | Exon 18 | TGTTTTAATTCAATCCCAC | 83 | 7832 | 7850 | 5-9-5 | 723 |
| 599373 | 2598 | 2616 | Exon 18 | CTGTTTTAATTCAATCCCA | 81 | 7833 | 7851 | 5-9-5 | 724 |
| 599374 | 2599 | 2617 | Exon 18 | GCTGTTTTAATTCAATCCC | 26 | 7834 | 7852 | 5-9-5 | 725 |
| 599375 | 2600 | 2618 | Exon 18 | AGCTGTTTTAATTCAATCC | 26 | 7835 | 7853 | 5-9-5 | 726 |
| 599376 | 2601 | 2619 | Exon 18 | CAGCTGTTTTAATTCAATC | 62 | 7836 | 7854 | 5-9-5 | 727 |
| 599377 | 2602 | 2620 | Exon 18 | GCAGCTGTTTTAATTCAAT | 21 | 7837 | 7855 | 5-9-5 | 728 |
| 599378 | 2603 | 2621 | Exon 18 | CGCAGCTGTTTTAATTCAA | 90 | 7838 | 7856 | 5-9-5 | 729 |
| 532917 | 2604 | 2623 | Exon 18 | GTCGCAGCTGTTTTAATTCA | 95 | 7839 | 7858 | 5-10-5 | 867 |
| 599379 | 2604 | 2622 | Exon 18 | TCGCAGCTGTTTTAATTCA | 88 | 7839 | 7857 | 5-9-5 | 730 |
| 599380 | 2605 | 2623 | Exon 18 | GTCGCAGCTGTTTTAATTC | 37 | 7840 | 7858 | 5-9-5 | 869 |
| 599381 | 2606 | 2624 | Exon 18 | TGTCGCAGCTGTTTTAATT | 33 | 7841 | 7859 | 5-9-5 | 732 |
| 599382 | 2607 | 2625 | Exon 18 | TTGTCGCAGCTGTTTTAAT | 81 | 7842 | 7860 | 5-9-5 | 733 |
| 599383 | 2608 | 2626 | Exon 18 | GTTGTCGCAGCTGTTTTAA | 54 | 7843 | 7861 | 5-9-5 | 734 |
| 599384 | 2609 | 2627 | Exon 18 | TGTTGTCGCAGCTGTTTTA | 85 | 7844 | 7862 | 5-9-5 | 873 |
| 599385 | 2610 | 2628 | Exon 18/ | TTGTTGTCGCAGCTGTTTT | 59 | n/a | n/a | 5-9-5 | 736 |
| | | | Repeat | | | | | | |
| 599386 | 2611 | 2629 | Exon 18/ | TTTGTTGTCGCAGCTGTTT | 81 | n/a | n/a | 5-9-5 | 737 |
| | | | Repeat | | | | | | |
| 599387 | 2612 | 2630 | Exon 18/ | TTTTGTTGTCGCAGCTGTT | 80 | n/a | n/a | 5-9-5 | 738 |
| | | | Repeat | | | | | | |
| 599388 | 2613 | 2631 | Exon 18/ | TTTTTGTTGTCGCAGCTGT | 84 | n/a | n/a | 5-9-5 | 739 |
| | | | Repeat | | | | | | |

### Example 120: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. Cultured HepG2 cells at a density of 20,000 cells per well were transfected using electroporation with 1,000 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed deoxy, MOE and (S)-cEt oligonucleotides. The deoxy, MOE and (S)-cEt oligonucleotides are 16 nucleosides in length wherein the nucleoside have either a MOE sugar modification, an (S)-cEt sugar modification, or a deoxy modification. The ‘Chemistry’ column describes the sugar modifications of each oligonucleotide. ‘k’ indicates an (S)-cEt sugar modification; ‘d’ indicates deoxyribose; and ‘e’ indicates a MOE modification.

“Start site” indicates the 5′-most nucleoside to which the gapmer is targeted in the human gene sequence. “Stop site” indicates the 3′-most nucleoside to which the gapmer is targeted human gene sequence. Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 10000 complementarity.

TABLE 141
Inhibition of CFB mRNA by deoxy, MOE and (S)-cEt oligonucleotides targeting SEQ ID NO: 1
or SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | start | stop | Target | | inhi- | start | stop | | ID |
| NO | site | site | region | Sequence | bition | site | site | Motif | NO: |
| 599513 | 2551 | 2566 | Exon 18 | AAACCCAAATCCTCAT | 11 | 7786 | 7801 | ekkeekkddddddddk | 557 |
| 599514 | 2553 | 2568 | Exon 18 | GAAAACCCAAATCCTC | 13 | 7788 | 7803 | ekkeekkddddddddk | 801 |
| 599515 | 2555 | 2570 | Exon 18 | TAGAAAACCCAAATCC | 54 | 7790 | 7805 | ekkeekkddddddddk | 559 |
| 599516 | 2559 | 2574 | Exon 18 | CTTATAGAAAACCCAA | 16 | 7794 | 7809 | ekkeekkddddddddk | 561 |
| 599517 | 2560 | 2575 | Exon 18 | CCTTATAGAAAACCCA | 29 | 7795 | 7810 | ekkeekkddddddddk | 562 |
| 599518 | 2561 | 2576 | Exon 18 | CCCTTATAGAAAACCC | 55 | 7796 | 7811 | ekkeekkddddddddk | 563 |
| 599519 | 2562 | 2577 | Exon 18 | CCCCTTATAGAAAACC | 31 | 7797 | 7812 | ekkeekkddddddddk | 564 |
| 599520 | 2563 | 2578 | Exon 18 | ACCCCTTATAGAAAAC | 14 | 7798 | 7813 | ekkeekkddddddddk | 565 |
| 599521 | 2564 | 2579 | Exon 18 | AACCCCTTATAGAAAA | 9 | 7799 | 7814 | ekkeekkddddddddk | 566 |
| 599522 | 2565 | 2580 | Exon 18 | AAACCCCTTATAGAAA | 8 | 7800 | 7815 | ekkeekkddddddddk | 567 |
| 599523 | 2566 | 2581 | Exon 18 | GAAACCCCTTATAGAA | 6 | 7801 | 7816 | ekkeekkddddddddk | 568 |
| 599524 | 2567 | 2582 | Exon 18 | GGAAACCCCTTATAGA | 14 | 7802 | 7817 | ekkeekkddddddddk | 569 |
| 599525 | 2568 | 2583 | Exon 18 | AGGAAACCCCTTATAG | 6 | 7803 | 7818 | ekkeekkddddddddk | 570 |
| 599526 | 2569 | 2584 | Exon 18 | CAGGAAACCCCTTATA | 16 | 7804 | 7819 | ekkeekkddddddddk | 571 |
| 599527 | 2570 | 2585 | Exon 18 | GCAGGAAACCCCTTAT | 0 | 7805 | 7820 | ekkeekkddddddddk | 572 |
| 599528 | 2571 | 2586 | Exon 18 | AGCAGGAAACCCCTTA | 6 | 7806 | 7821 | ekkeekkddddddddk | 573 |
| 599529 | 2572 | 2587 | Exon 18 | CAGCAGGAAACCCCTT | 6 | 7807 | 7822 | ekkeekkddddddddk | 574 |
| 599530 | 2574 | 2589 | Exon 18 | TCCAGCAGGAAACCCC | 29 | 7809 | 7824 | ekkeekkddddddddk | 576 |
| 599531 | 2575 | 2590 | Exon 18 | GTCCAGCAGGAAACCC | 64 | 7810 | 7825 | ekkeekkddddddddk | 577 |
| 599532 | 2576 | 2591 | Exon 18 | TGTCCAGCAGGAAACC | 43 | 7811 | 7826 | ekkeekkddddddddk | 578 |
| 599533 | 2577 | 2592 | Exon 18 | CTGTCCAGCAGGAAAC | 25 | 7812 | 7827 | ekkeekkddddddddk | 820 |
| 599534 | 2578 | 2593 | Exon 18 | CCTGTCCAGCAGGAAA | 12 | 7813 | 7828 | ekkeekkddddddddk | 580 |
| 599535 | 2580 | 2595 | Exon 18 | CCCCTGTCCAGCAGGA | 16 | 7815 | 7830 | ekkeekkddddddddk | 582 |
| 599536 | 2582 | 2597 | Exon 18 | CGCCCCTGTCCAGCAG | 27 | 7817 | 7832 | ekkeekkddddddddk | 584 |
| 599537 | 2583 | 2598 | Exon 18 | ACGCCCCTGTCCAGCA | 35 | 7818 | 7833 | ekkeekkddddddddk | 585 |
| 599538 | 2584 | 2599 | Exon 18 | CACGCCCCTGTCCAGC | 26 | 7819 | 7834 | ekkeekkddddddddk | 586 |
| 599539 | 2585 | 2600 | Exon 18 | CCACGCCCCTGTCCAG | 33 | 7820 | 7835 | ekkeekkddddddddk | 587 |
| 599540 | 2586 | 2601 | Exon 18 | CCCACGCCCCTGTCCA | 27 | 7821 | 7836 | ekkeekkddddddddk | 588 |
| 599541 | 2587 | 2602 | Exon 18 | TCCCACGCCCCTGTCC | 52 | 7822 | 7837 | ekkeekkddddddddk | 589 |
| 599542 | 2588 | 2603 | Exon 18 | ATCCCACGCCCCTGTC | 16 | 7823 | 7838 | ekkeekkddddddddk | 590 |
| 599543 | 2589 | 2604 | Exon 18 | AATCCCACGCCCCTGT | 19 | 7824 | 7839 | ekkeekkddddddddk | 591 |
| 599544 | 2590 | 2605 | Exon 18 | CAATCCCACGCCCCTG | 33 | 7825 | 7840 | ekkeekkddddddddk | 831 |
| 599545 | 2591 | 2606 | Exon 18 | TCAATCCCACGCCCCT | 24 | 7826 | 7841 | ekkeekkddddddddk | 593 |
| 599546 | 2592 | 2607 | Exon 18 | TTCAATCCCACGCCCC | 54 | 7827 | 7842 | ekkeekkddddddddk | 594 |
| 599547 | 2593 | 2608 | Exon 18 | ATTCAATCCCACGCCC | 87 | 7828 | 7843 | ekkeekkddddddddk | 595 |
| 599548 | 2594 | 2609 | Exon 18 | AATTCAATCCCACGCC | 79 | 7829 | 7844 | ekkeekkddddddddk | 596 |
| 599549 | 2595 | 2610 | Exon 18 | TAATTCAATCCCACGC | 62 | 7830 | 7845 | ekkeekkddddddddk | 597 |
| 599550 | 2596 | 2611 | Exon 18 | TTAATTCAATCCCACG | 52 | 7831 | 7846 | ekkeekkddddddddk | 598 |
| 599551 | 2597 | 2612 | Exon 18 | TTTAATTCAATCCCAC | 27 | 7832 | 7847 | ekkeekkddddddddk | 599 |
| 599577 | 2597 | 2613 | Exon 18 | TTTTAATTCAATCCCAC | 90 | 7832 | 7848 | eeekkddddddddkeee | 662 |
| 599552 | 2598 | 2613 | Exon 18 | TTTTAATTCAATCCCA | 92 | 7833 | 7848 | ekkeekkddddddddk | 600 |
| 599578 | 2598 | 2614 | Exon 18 | GTTTTAATTCAATCCCA | 88 | 7833 | 7849 | eeekkddddddddkeee | 663 |
| 599553 | 2599 | 2614 | Exon 18 | GTTTTAATTCAATCCC | 91 | 7834 | 7849 | ekkeekkddddddddk | 601 |
| 599579 | 2599 | 2615 | Exon 18 | TGTTTTAATTCAATCCC | 79 | 7834 | 7850 | eeekkddddddddkeee | 664 |
| 599554 | 2600 | 2615 | Exon 18 | TGTTTTAATTCAATCC | 90 | 7835 | 7850 | ekkeekkddddddddk | 602 |
| 599580 | 2600 | 2616 | Exon 18 | CTGTTTTAATTCAATCC | 79 | 7835 | 7851 | eeekkddddddddkeee | 665 |
| 599555 | 2601 | 2616 | Exon 18 | CTGTTTTAATTCAATC | 79 | 7836 | 7851 | ekkeekkddddddddk | 846 |
| 599581 | 2601 | 2617 | Exon 18 | GCTGTTTTAATTCAATC | 90 | 7836 | 7852 | eeekkddddddddkeee | 666 |
| 599556 | 2602 | 2617 | Exon 18 | GCTGTTTTAATTCAAT | 47 | 7837 | 7852 | ekkeekkddddddddk | 604 |
| 599582 | 2602 | 2618 | Exon 18 | AGCTGTTTTAATTCAAT | 89 | 7837 | 7853 | eeekkddddddddkeee | 849 |
| 599557 | 2603 | 2618 | Exon 18 | AGCTGTTTTAATTCAA | 67 | 7838 | 7853 | ekkeekkddddddddk | 850 |
| 599583 | 2603 | 2619 | Exon 18 | CAGCTGTTTTAATTCAA | 49 | 7838 | 7854 | eeekkddddddddkeee | 668 |
| 532917 | 2604 | 2623 | Exon 18 | GTCGCAGCTGTTTTAAT | 78 | 7839 | 7858 | eeeeeddddddddddeee | 317 |
| | | | | TCA | | | | ee | |
| 599558 | 2604 | 2619 | Exon 18 | CAGCTGTTTTAATTCA | 80 | 7839 | 7854 | ekkeekkddddddddk | 606 |
| 599584 | 2604 | 2620 | Exon 18 | GCAGCTGTTTTAATTCA | 66 | 7839 | 7855 | eeekkddddddddkeee | 669 |
| 599559 | 2605 | 2620 | Exon 18 | GCAGCTGTTTTAATTC | 38 | 7840 | 7855 | ekkeekkddddddddk | 607 |
| 599585 | 2605 | 2621 | Exon 18 | CGCAGCTGTTTTAATTC | 80 | 7840 | 7856 | eeekkddddddddkeee | 670 |
| 599560 | 2606 | 2621 | Exon 18 | CGCAGCTGTTTTAATT | 16 | 7841 | 7856 | ekkeekkddddddddk | 608 |
| 599586 | 2606 | 2622 | Exon 18 | TCGCAGCTGTTTTAATT | 78 | 7841 | 7857 | eeekkddddddddkeee | 671 |
| 599561 | 2607 | 2622 | Exon 18 | TCGCAGCTGTTTTAAT | 58 | 7842 | 7857 | ekkeekkddddddddk | 609 |
| 599587 | 2607 | 2623 | Exon 18 | GTCGCAGCTGTTTTAAT | 81 | 7842 | 7858 | eeekkddddddddkeee | 672 |
| 588860 | 2608 | 2623 | Exon 18 | GTCGCAGCTGTTTTAA | 92 | 7843 | 7858 | eekdddddddddddke | 610 |
| 599562 | 2608 | 2623 | Exon 18 | GTCGCAGCTGTTTTAA | 78 | 7843 | 7858 | ekkeekkddddddddk | 610 |
| 599588 | 2608 | 2624 | Exon 18 | TGTCGCAGCTGTTTTAA | 81 | 7843 | 7859 | eeekkddddddddkeee | 673 |
| 599563 | 2609 | 2624 | Exon 18 | TGTCGCAGCTGTTTTA | 86 | 7844 | 7859 | ekkeekkddddddddk | 611 |
| 599589 | 2609 | 2625 | Exon 18 | TTGTCGCAGCTGTTTTA | 75 | 7844 | 7860 | eeekkddddddddkeee | 674 |
| 599564 | 2610 | 2625 | Exon 18 | TTGTCGCAGCTGTTTT | 75 | 7845 | 7860 | ekkeekkddddddddk | 612 |
| 599590 | 2610 | 2626 | Exon 18 | GTTGTCGCAGCTGTTTT | 88 | 7845 | 7861 | eeekkddddddddkeee | 675 |
| 599565 | 2611 | 2626 | Exon 18 | GTTGTCGCAGCTGTTT | 65 | 7846 | 7861 | ekkeekkddddddddk | 613 |
| 599591 | 2611 | 2627 | Exon 18 | TGTTGTCGCAGCTGTTT | 94 | 7846 | 7862 | eeekkddddddddkeee | 676 |
| 599566 | 2612 | 2627 | Exon 18 | TGTTGTCGCAGCTGTT | 72 | 7847 | 7862 | ekkeekkddddddddk | 614 |
| 599592 | 2612 | 2628 | Exon 18/ | TTGTTGTCGCAGCTGTT | 90 | n/a | n/a | eeekkddddddddkeee | 677 |
| | | | Repeat | | | | | | |
| 599567 | 2613 | 2628 | Exon 18/ | TTGTTGTCGCAGCTGT | 82 | n/a | n/a | ekkeekkddddddddk | 615 |
| | | | Repeat | | | | | | |
| 599593 | 2613 | 2629 | Exon 18/ | TTTGTTGTCGCAGCTGT | 95 | n/a | n/a | eeekkddddddddkeee | 678 |
| | | | Repeat | | | | | | |
| 599568 | 2614 | 2629 | Exon 18/ | TTTGTTGTCGCAGCTG | 92 | n/a | n/a | ekkeekkddddddddk | 616 |
| | | | Repeat | | | | | | |
| 599594 | 2614 | 2630 | Exon 18/ | TTTTGTTGTCGCAGCTG | 86 | n/a | n/a | eeekkddddddddkeee | 679 |
| | | | Repeat | | | | | | |
| 599569 | 2615 | 2630 | Exon 18/ | TTTTGTTGTCGCAGCT | 89 | n/a | n/a | ekkeekkddddddddk | 617 |
| | | | Repeat | | | | | | |
| 599595 | 2615 | 2631 | Exon 18/ | TTTTTGTTGTCGCAGCT | 76 | n/a | n/a | eeekkddddddddkeee | 680 |
| | | | Repeat | | | | | | |
| 599570 | 2616 | 2631 | Exon 18/ | TTTTTGTTGTCGCAGC | 95 | n/a | n/a | ekkeekkddddddddk | 618 |
| | | | Repeat | | | | | | |

### Example 121: Antisense Inhibition of Human Complement Factor B (CFB) in HepG2 Cells by MOE Gapmers

Additional antisense oligonucleotides were designed targeting human Complement Factor B (CFB) nucleic acid and were tested for their effects on CFB mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 500 nM antisense oligonucleotide. After a treatment period of approximately 24 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The newly designed chimeric antisense oligonucleotides in the Tables below were designed as deoxy, MOE and (S)-cEt oligonucleotides, or as 5-8-5 MOE, 5-9-5 MOE, 5-10-5 MOE, 6-7-6 MOE, 3-10-5 MOE, or 6-8-6 MOE gapmers.

The deoxy, MOE and (S)-cEt oligonucleotides are 16 nucleosides in length wherein the nucleoside have either a MOE sugar modification, an (S)-cEt sugar modification, or a deoxy modification. The ‘Chemistry’ column describes the sugar modifications of each oligonucleotide. ‘k’ indicates an (S)-cEt sugar modification; ‘d’ indicates deoxyribose; and ‘e’ indicates a MOE modification. The 5-8-5 MOE gapmers are 18 nucleosides in length, wherein the central gap segment comprises of eight 2′-deoxynucleosides and is flanked by wing segments on the 5′ direction and the 3′ direction comprising five nucleosides each. The 5-9-5 MOE gapmers are 19 nucleosides in length, wherein the central gap segment comprises of nine 2′-deoxynucleosides and is flanked by wing segments on the 5′ direction and the 3′ direction comprising five nucleosides each. The 5-10-5 MOE gapmers are 20 nucleosides in length, wherein the central gap segment comprises of ten 2′-deoxynucleosides and is flanked by wing segments on the 5′ direction and the 3′ direction comprising five nucleosides each. The 3-10-5 MOE gapmers are 18 nucleosides in length, wherein the central gap segment comprises of ten 2′-deoxynucleosides and is flanked by wing segments on the 5′ direction and the 3′ direction comprising three and five nucleosides respectively. The 6-7-6 MOE gapmers are 19 nucleosides in length, wherein the central gap segment comprises of seven 2′-deoxynucleosides and is flanked by wing segments on the 5′ direction and the 3′ direction comprising six nucleosides each. The 6-8-6 MOE gapmers are 20 nucleosides in length, wherein the central gap segment comprises of eight 2′-deoxynucleosides and is flanked by wing segments on the 5′ direction and the 3′ direction comprising six nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines.

“Start site” indicates the 5′-most nucleoside to which the gapmer is targeted in the human gene sequence. “Stop site” indicates the 3′-most nucleoside to which the gapmer is targeted human gene sequence.

Each gapmer listed in the Tables below is targeted to either the human CFB mRNA, designated herein as SEQ ID NO: 1 (GENBANK Accession No. NM_001710.5) or the human CFB genomic sequence, designated herein as SEQ ID NO: 2 (GENBANK Accession No. NT_007592.15 truncated from nucleotides 31852000 to 31861000), or both. ‘n/a’ indicates that the antisense oligonucleotide does not target that particular gene sequence with 100% complementarity.

TABLE 142
Inhibition of CFB mRNA by deoxy, MOE and (S)-cEt oligonucleotides targeting SEQ ID NO: 1
or SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | start | stop | Target | | inhi- | start | stop | | ID |
| NO | site | site | region | Sequence | bition | site | site | Motif | NO: |
| 601152 | 2551 | 2566 | Exon 18 | AAACCCAAATCCTCAT | 22 | 7786 | 7801 | eekkdddddddddkee | 557 |
| 601218 | 2551 | 2566 | Exon 18 | AAACCCAAATCCTCAT | 21 | 7786 | 7801 | edkkdddddddddeee | 557 |
| 601153 | 2552 | 2567 | Exon 18 | AAAACCCAAATCCTCA | 27 | 7787 | 7802 | eekkdddddddddkee | 800 |
| 601219 | 2552 | 2567 | Exon 18 | AAAACCCAAATCCTCA | 19 | 7787 | 7802 | edkkdddddddddeee | 800 |
| 601154 | 2553 | 2568 | Exon 18 | GAAAACCCAAATCCTC | 23 | 7788 | 7803 | eekkdddddddddkee | 558 |
| 601220 | 2553 | 2568 | Exon 18 | GAAAACCCAAATCCTC | 24 | 7788 | 7803 | edkkdddddddddeee | 558 |
| 601155 | 2554 | 2569 | Exon 18 | AGAAAACCCAAATCCT | 20 | 7789 | 7804 | eekkdddddddddkee | 801 |
| 601221 | 2554 | 2569 | Exon 18 | AGAAAACCCAAATCCT | 0 | 7789 | 7804 | edkkdddddddddeee | 801 |
| 601156 | 2555 | 2570 | Exon 18 | TAGAAAACCCAAATCC | 11 | 7790 | 7805 | eekkdddddddddkee | 559 |
| 601222 | 2555 | 2570 | Exon 18 | TAGAAAACCCAAATCC | 23 | 7790 | 7805 | edkkdddddddddeee | 559 |
| 601157 | 2556 | 2571 | Exon 18 | ATAGAAAACCCAAATC | 9 | 7791 | 7806 | eekkdddddddddkee | 560 |
| 601223 | 2556 | 2571 | Exon 18 | ATAGAAAACCCAAATC | 0 | 7791 | 7806 | edkkdddddddddeee | 560 |
| 601158 | 2557 | 2572 | Exon 18 | TATAGAAAACCCAAAT | 0 | 7792 | 7807 | eekkdddddddddkee | 802 |
| 601224 | 2557 | 2572 | Exon 18 | TATAGAAAACCCAAAT | 0 | 7792 | 7807 | edkkdddddddddeee | 802 |
| 601159 | 2558 | 2573 | Exon 18 | TTATAGAAAACCCAAA | 2 | 7793 | 7808 | eekkdddddddddkee | 803 |
| 601225 | 2558 | 2573 | Exon 18 | TTATAGAAAACCCAAA | 0 | 7793 | 7808 | edkkdddddddddeee | 803 |
| 601160 | 2559 | 2574 | Exon 18 | CTTATAGAAAACCCAA | 0 | 7794 | 7809 | eekkdddddddddkee | 561 |
| 601226 | 2559 | 2574 | Exon 18 | CTTATAGAAAACCCAA | 0 | 7794 | 7809 | edkkdddddddddeee | 561 |
| 601161 | 2560 | 2575 | Exon 18 | CCTTATAGAAAACCCA | 1 | 7795 | 7810 | eekkdddddddddkee | 562 |
| 601227 | 2560 | 2575 | Exon 18 | CCTTATAGAAAACCCA | 14 | 7795 | 7810 | edkkdddddddddeee | 562 |
| 601162 | 2561 | 2576 | Exon 18 | CCCTTATAGAAAACCC | 9 | 7796 | 7811 | eekkdddddddddkee | 563 |
| 601228 | 2561 | 2576 | Exon 18 | CCCTTATAGAAAACCC | 9 | 7796 | 7811 | edkkdddddddddeee | 563 |
| 601163 | 2562 | 2577 | Exon 18 | CCCCTTATAGAAAACC | 0 | 7797 | 7812 | eekkdddddddddkee | 564 |
| 601164 | 2563 | 2578 | Exon 18 | ACCCCTTATAGAAAAC | 3 | 7798 | 7813 | eekkdddddddddkee | 565 |
| 601165 | 2564 | 2579 | Exon 18 | AACCCCTTATAGAAAA | 0 | 7799 | 7814 | eekkdddddddddkee | 566 |
| 601166 | 2565 | 2580 | Exon 18 | AAACCCCTTATAGAAA | 0 | 7800 | 7815 | eekkdddddddddkee | 567 |
| 601167 | 2566 | 2581 | Exon 18 | GAAACCCCTTATAGAA | 0 | 7801 | 7816 | eekkdddddddddkee | 568 |
| 601168 | 2567 | 2582 | Exon 18 | GGAAACCCCTTATAGA | 0 | 7802 | 7817 | eekkdddddddddkee | 569 |
| 601169 | 2568 | 2583 | Exon 18 | AGGAAACCCCTTATAG | 0 | 7803 | 7818 | eekkdddddddddkee | 570 |
| 601170 | 2569 | 2584 | Exon 18 | CAGGAAACCCCTTATA | 10 | 7804 | 7819 | eekkdddddddddkee | 571 |
| 601171 | 2570 | 2585 | Exon 18 | GCAGGAAACCCCTTAT | 9 | 7805 | 7820 | eekkdddddddddkee | 572 |
| 601172 | 2571 | 2586 | Exon 18 | AGCAGGAAACCCCTTA | 15 | 7806 | 7821 | eekkdddddddddkee | 573 |
| 601173 | 2572 | 2587 | Exon 18 | CAGCAGGAAACCCCTT | 29 | 7807 | 7822 | eekkdddddddddkee | 574 |
| 601174 | 2573 | 2588 | Exon 18 | CCAGCAGGAAACCCCT | 25 | 7808 | 7823 | eekkdddddddddkee | 575 |
| 601175 | 2574 | 2589 | Exon 18 | TCCAGCAGGAAACCCC | 15 | 7809 | 7824 | eekkdddddddddkee | 576 |
| 601176 | 2575 | 2590 | Exon 18 | GTCCAGCAGGAAACCC | 18 | 7810 | 7825 | eekkdddddddddkee | 577 |
| 601177 | 2576 | 2591 | Exon 18 | TGTCCAGCAGGAAACC | 10 | 7811 | 7826 | eekkdddddddddkee | 578 |
| 601178 | 2577 | 2592 | Exon 18 | CTGTCCAGCAGGAAAC | 11 | 7812 | 7827 | eekkdddddddddkee | 579 |
| 601179 | 2578 | 2593 | Exon 18 | CCTGTCCAGCAGGAAA | 19 | 7813 | 7828 | eekkdddddddddkee | 580 |
| 601180 | 2579 | 2594 | Exon 18 | CCCTGTCCAGCAGGAA | 7 | 7814 | 7829 | eekkdddddddddkee | 581 |
| 601181 | 2580 | 2595 | Exon 18 | CCCCTGTCCAGCAGGA | 3 | 7815 | 7830 | eekkdddddddddkee | 582 |
| 601182 | 2581 | 2596 | Exon 18 | GCCCCTGTCCAGCAGG | 0 | 7816 | 7831 | eekkdddddddddkee | 583 |
| 601183 | 2582 | 2597 | Exon 18 | CGCCCCTGTCCAGCAG | 4 | 7817 | 7832 | eekkdddddddddkee | 584 |
| 601184 | 2583 | 2598 | Exon 18 | ACGCCCCTGTCCAGCA | 14 | 7818 | 7833 | eekkdddddddddkee | 585 |
| 601185 | 2584 | 2599 | Exon 18 | CACGCCCCTGTCCAGC | 26 | 7819 | 7834 | eekkdddddddddkee | 586 |
| 601186 | 2585 | 2600 | Exon 18 | CCACGCCCCTGTCCAG | 8 | 7820 | 7835 | eekkdddddddddkee | 587 |
| 601187 | 2586 | 2601 | Exon 18 | CCCACGCCCCTGTCCA | 18 | 7821 | 7836 | eekkdddddddddkee | 588 |
| 601188 | 2587 | 2602 | Exon 18 | TCCCACGCCCCTGTCC | 20 | 7822 | 7837 | eekkdddddddddkee | 589 |
| 601189 | 2588 | 2603 | Exon 18 | ATCCCACGCCCCTGTC | 12 | 7823 | 7838 | eekkdddddddddkee | 590 |
| 601190 | 2589 | 2604 | Exon 18 | AATCCCACGCCCCTGT | 33 | 7824 | 7839 | eekkdddddddddkee | 591 |
| 601191 | 2590 | 2605 | Exon 18 | CAATCCCACGCCCCTG | 52 | 7825 | 7840 | eekkdddddddddkee | 592 |
| 601192 | 2591 | 2606 | Exon 18 | TCAATCCCACGCCCCT | 46 | 7826 | 7841 | eekkdddddddddkee | 593 |
| 601193 | 2592 | 2607 | Exon 18 | TTCAATCCCACGCCCC | 30 | 7827 | 7842 | eekkdddddddddkee | 594 |
| 601194 | 2593 | 2608 | Exon 18 | ATTCAATCCCACGCCC | 41 | 7828 | 7843 | eekkdddddddddkee | 595 |
| 601195 | 2594 | 2609 | Exon 18 | AATTCAATCCCACGCC | 40 | 7829 | 7844 | eekkdddddddddkee | 596 |
| 601196 | 2595 | 2610 | Exon 18 | TAATTCAATCCCACGC | 71 | 7830 | 7845 | eekkdddddddddkee | 597 |
| 601197 | 2596 | 2611 | Exon 18 | TTAATTCAATCCCACG | 42 | 7831 | 7846 | eekkdddddddddkee | 598 |
| 601198 | 2597 | 2612 | Exon 18 | TTTAATTCAATCCCAC | 63 | 7832 | 7847 | eekkdddddddddkee | 599 |
| 601199 | 2598 | 2613 | Exon 18 | TTTTAATTCAATCCCA | 51 | 7833 | 7848 | eekkdddddddddkee | 600 |
| 601200 | 2599 | 2614 | Exon 18 | GTTTTAATTCAATCCC | 65 | 7834 | 7849 | eekkdddddddddkee | 601 |
| 601201 | 2600 | 2615 | Exon 18 | TGTTTTAATTCAATCC | 49 | 7835 | 7850 | eekkdddddddddkee | 602 |
| 601202 | 2601 | 2616 | Exon 18 | CTGTTTTAATTCAATC | 33 | 7836 | 7851 | eekkdddddddddkee | 603 |
| 601203 | 2602 | 2617 | Exon 18 | GCTGTTTTAATTCAAT | 63 | 7837 | 7852 | eekkdddddddddkee | 604 |
| 601204 | 2603 | 2618 | Exon 18 | AGCTGTTTTAATTCAA | 69 | 7838 | 7853 | eekkdddddddddkee | 605 |
| 532917 | 2604 | 2623 | Exon 18 | GTCGCAGCTGTTTTAATT | 73 | 7839 | 7858 | eeeeeddddddddddeeee | 317 |
| | | | | CA | | | | e | |
| 601205 | 2604 | 2619 | Exon 18 | CAGCTGTTTTAATTCA | 51 | 7839 | 7854 | eekkdddddddddkee | 606 |
| 601206 | 2605 | 2620 | Exon 18 | GCAGCTGTTTTAATTC | 43 | 7840 | 7855 | eekkdddddddddkee | 607 |
| 601207 | 2606 | 2621 | Exon 18 | CGCAGCTGTTTTAATT | 52 | 7841 | 7856 | eekkdddddddddkee | 608 |
| 601208 | 2607 | 2622 | Exon 18 | TCGCAGCTGTTTTAAT | 61 | 7842 | 7857 | eekkdddddddddkee | 609 |
| 588860 | 2608 | 2623 | Exon 18 | GTCGCAGCTGTTTTAA | 75 | 7843 | 7858 | eekdddddddddddke | 610 |
| 601209 | 2608 | 2623 | Exon 18 | GTCGCAGCTGTTTTAA | 73 | 7843 | 7858 | eekkdddddddddkee | 610 |
| 601210 | 2609 | 2624 | Exon 18 | TGTCGCAGCTGTTTTA | 80 | 7844 | 7859 | eekkdddddddddkee | 611 |
| 601211 | 2610 | 2625 | Exon 18 | TTGTCGCAGCTGTTTT | 64 | 7845 | 7860 | eekkdddddddddkee | 612 |
| 601212 | 2611 | 2626 | Exon 18 | GTTGTCGCAGCTGTTT | 86 | 7846 | 7861 | eekkdddddddddkee | 613 |
| 601213 | 2612 | 2627 | Exon 18 | TGTTGTCGCAGCTGTT | 87 | 7847 | 7862 | eekkdddddddddkee | 614 |
| 601214 | 2613 | 2628 | Exon 18/ | TTGTTGTCGCAGCTGT | 84 | n/a | n/a | eekkdddddddddkee | 615 |
| | | | Repeat | | | | | | |
| 601215 | 2614 | 2629 | Exon 18/ | TTTGTTGTCGCAGCTG | 78 | n/a | n/a | eekkdddddddddkee | 616 |
| | | | Repeat | | | | | | |
| 601216 | 2615 | 2630 | Exon 18/ | TTTTGTTGTCGCAGCT | 73 | n/a | n/a | eekkdddddddddkee | 617 |
| | | | Repeat | | | | | | |
| 601217 | 2616 | 2631 | Exon 18/ | TTTTTGTTGTCGCAGC | 66 | n/a | n/a | eekkdddddddddkee | 618 |
| | | | Repeat | | | | | | |

TABLE 143
Inhibition of CFB mRNA by deoxy, MOE and (S)-cEt oligonucleotides targeting SEQ ID NO: 1
or SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | start | stop | Target | | inhi- | start | stop | | ID |
| NO | site | site | region | Sequence | bition | site | site | Motif | NO: |
| 601284 | 2551 | 2566 | Exon 18 | AAACCCAAATCCTCAT | 8 | 7786 | 7801 | ekkdddddddddkeee | 557 |
| 601285 | 2552 | 2567 | Exon 18 | AAAACCCAAATCCTCA | 15 | 7787 | 7802 | ekkdddddddddkeee | 800 |
| 601286 | 2553 | 2568 | Exon 18 | GAAAACCCAAATCCTC | 21 | 7788 | 7803 | ekkdddddddddkeee | 558 |
| 601287 | 2554 | 2569 | Exon 18 | AGAAAACCCAAATCCT | 9 | 7789 | 7804 | ekkdddddddddkeee | 801 |
| 601288 | 2555 | 2570 | Exon 18 | TAGAAAACCCAAATCC | 0 | 7790 | 7805 | ekkdddddddddkeee | 559 |
| 601289 | 2556 | 2571 | Exon 18 | ATAGAAAACCCAAATC | 40 | 7791 | 7806 | ekkdddddddddkeee | 560 |
| 601290 | 2557 | 2572 | Exon 18 | TATAGAAAACCCAAAT | 16 | 7792 | 7807 | ekkdddddddddkeee | 802 |
| 601291 | 2558 | 2573 | Exon 18 | TTATAGAAAACCCAAA | 15 | 7793 | 7808 | ekkdddddddddkeee | 803 |
| 601292 | 2559 | 2574 | Exon 18 | CTTATAGAAAACCCAA | 5 | 7794 | 7809 | ekkdddddddddkeee | 561 |
| 601293 | 2560 | 2575 | Exon 18 | CCTTATAGAAAACCCA | 15 | 7795 | 7810 | ekkdddddddddkeee | 562 |
| 601294 | 2561 | 2576 | Exon 18 | CCCTTATAGAAAACCC | 3 | 7796 | 7811 | ekkdddddddddkeee | 563 |
| 601229 | 2562 | 2577 | Exon 18 | CCCCTTATAGAAAACC | 15 | 7797 | 7812 | edkddddddddddeee | 564 |
| 601295 | 2562 | 2577 | Exon 18 | CCCCTTATAGAAAACC | 5 | 7797 | 7812 | ekkdddddddddkeee | 564 |
| 601230 | 2563 | 2578 | Exon 18 | ACCCCTTATAGAAAAC | 14 | 7798 | 7813 | edkkdddddddddeee | 565 |
| 601296 | 2563 | 2578 | Exon 18 | ACCCCTTATAGAAAAC | 0 | 7798 | 7813 | ekkdddddddddkeee | 565 |
| 601231 | 2564 | 2579 | Exon 18 | AACCCCTTATAGAAAA | 14 | 7799 | 7814 | edkkdddddddddeee | 566 |
| 601297 | 2564 | 2579 | Exon 18 | AACCCCTTATAGAAAA | 14 | 7799 | 7814 | ekkdddddddddkeee | 566 |
| 601232 | 2565 | 2580 | Exon 18 | AAACCCCTTATAGAAA | 15 | 7800 | 7815 | edkddddddddddeee | 567 |
| 601298 | 2565 | 2580 | Exon 18 | AAACCCCTTATAGAAA | 7 | 7800 | 7815 | ekkdddddddddkeee | 567 |
| 601233 | 2566 | 2581 | Exon 18 | GAAACCCCTTATAGAA | 0 | 7801 | 7816 | edkddddddddddeee | 568 |
| 601299 | 2566 | 2581 | Exon 18 | GAAACCCCTTATAGAA | 0 | 7801 | 7816 | ekkdddddddddkeee | 568 |
| 601234 | 2567 | 2582 | Exon 18 | GGAAACCCCTTATAGA | 0 | 7802 | 7817 | edkddddddddddeee | 569 |
| 601300 | 2567 | 2582 | Exon 18 | GGAAACCCCTTATAGA | 9 | 7802 | 7817 | ekkdddddddddkeee | 569 |
| 601235 | 2568 | 2583 | Exon 18 | AGGAAACCCCTTATAG | 3 | 7803 | 7818 | edkddddddddddeee | 570 |
| 601301 | 2568 | 2583 | Exon 18 | AGGAAACCCCTTATAG | 14 | 7803 | 7818 | ekkdddddddddkeee | 570 |
| 601236 | 2569 | 2584 | Exon 18 | CAGGAAACCCCTTATA | 0 | 7804 | 7819 | edkkdddddddddeee | 571 |
| 601302 | 2569 | 2584 | Exon 18 | CAGGAAACCCCTTATA | 0 | 7804 | 7819 | ekkdddddddddkeee | 571 |
| 601237 | 2570 | 2585 | Exon 18 | GCAGGAAACCCCTTAT | 16 | 7805 | 7820 | edkkdddddddddeee | 572 |
| 601303 | 2570 | 2585 | Exon 18 | GCAGGAAACCCCTTAT | 16 | 7805 | 7820 | ekkdddddddddkeee | 572 |
| 601238 | 2571 | 2586 | Exon 18 | AGCAGGAAACCCCTTA | 11 | 7806 | 7821 | edkkdddddddddeee | 573 |
| 601304 | 2571 | 2586 | Exon 18 | AGCAGGAAACCCCTTA | 10 | 7806 | 7821 | ekkdddddddddkeee | 573 |
| 601239 | 2572 | 2587 | Exon 18 | CAGCAGGAAACCCCTT | 21 | 7807 | 7822 | edkkdddddddddeee | 574 |
| 601305 | 2572 | 2587 | Exon 18 | CAGCAGGAAACCCCTT | 7 | 7807 | 7822 | ekkdddddddddkeee | 574 |
| 601240 | 2573 | 2588 | Exon 18 | CCAGCAGGAAACCCCT | 6 | 7808 | 7823 | edkkdddddddddeee | 575 |
| 601241 | 2574 | 2589 | Exon 18 | TCCAGCAGGAAACCCC | 10 | 7809 | 7824 | edkkdddddddddeee | 576 |
| 601242 | 2575 | 2590 | Exon 18 | GTCCAGCAGGAAACCC | 19 | 7810 | 7825 | edkkdddddddddeee | 577 |
| 601243 | 2576 | 2591 | Exon 18 | TGTCCAGCAGGAAACC | 10 | 7811 | 7826 | edkkdddddddddeee | 578 |
| 601244 | 2577 | 2592 | Exon 18 | CTGTCCAGCAGGAAAC | 28 | 7812 | 7827 | edkkdddddddddeee | 579 |
| 601245 | 2578 | 2593 | Exon 18 | CCTGTCCAGCAGGAAA | 5 | 7813 | 7828 | edkkdddddddddeee | 580 |
| 601246 | 2579 | 2594 | Exon 18 | CCCTGTCCAGCAGGAA | 18 | 7814 | 7829 | edkkdddddddddeee | 581 |
| 601247 | 2580 | 2595 | Exon 18 | CCCCTGTCCAGCAGGA | 4 | 7815 | 7830 | edkkdddddddddeee | 582 |
| 601248 | 2581 | 2596 | Exon 18 | GCCCCTGTCCAGCAGG | 6 | 7816 | 7831 | edkkdddddddddeee | 583 |
| 601249 | 2582 | 2597 | Exon 18 | CGCCCCTGTCCAGCAG | 18 | 7817 | 7832 | edkkdddddddddeee | 584 |
| 601250 | 2583 | 2598 | Exon 18 | ACGCCCCTGTCCAGCA | 26 | 7818 | 7833 | edkkdddddddddeee | 585 |
| 601251 | 2584 | 2599 | Exon 18 | CACGCCCCTGTCCAGC | 27 | 7819 | 7834 | edkkdddddddddeee | 586 |
| 601252 | 2585 | 2600 | Exon 18 | CCACGCCCCTGTCCAG | 21 | 7820 | 7835 | edkkdddddddddeee | 587 |
| 601253 | 2586 | 2601 | Exon 18 | CCCACGCCCCTGTCCA | 0 | 7821 | 7836 | edkkdddddddddeee | 588 |
| 601254 | 2587 | 2602 | Exon 18 | TCCCACGCCCCTGTCC | 31 | 7822 | 7837 | edkkdddddddddeee | 589 |
| 601255 | 2588 | 2603 | Exon 18 | ATCCCACGCCCCTGTC | 3 | 7823 | 7838 | edkkdddddddddeee | 590 |
| 601256 | 2589 | 2604 | Exon 18 | AATCCCACGCCCCTGT | 21 | 7824 | 7839 | edkkdddddddddeee | 591 |
| 601257 | 2590 | 2605 | Exon 18 | CAATCCCACGCCCCTG | 47 | 7825 | 7840 | edkkdddddddddeee | 592 |
| 601258 | 2591 | 2606 | Exon 18 | TCAATCCCACGCCCCT | 48 | 7826 | 7841 | edkkdddddddddeee | 593 |
| 601259 | 2592 | 2607 | Exon 18 | TTCAATCCCACGCCCC | 38 | 7827 | 7842 | edkkdddddddddeee | 594 |
| 601260 | 2593 | 2608 | Exon 18 | ATTCAATCCCACGCCC | 33 | 7828 | 7843 | edkkdddddddddeee | 595 |
| 601261 | 2594 | 2609 | Exon 18 | AATTCAATCCCACGCC | 17 | 7829 | 7844 | edkkdddddddddeee | 596 |
| 601262 | 2595 | 2610 | Exon 18 | TAATTCAATCCCACGC | 40 | 7830 | 7845 | edkkdddddddddeee | 597 |
| 601263 | 2596 | 2611 | Exon 18 | TTAATTCAATCCCACG | 31 | 7831 | 7846 | edkkdddddddddeee | 598 |
| 601264 | 2597 | 2612 | Exon 18 | TTTAATTCAATCCCAC | 72 | 7832 | 7847 | edkkdddddddddeee | 599 |
| 601265 | 2598 | 2613 | Exon 18 | TTTTAATTCAATCCCA | 48 | 7833 | 7848 | edkkdddddddddeee | 600 |
| 601266 | 2599 | 2614 | Exon 18 | GTTTTAATTCAATCCC | 64 | 7834 | 7849 | edkkdddddddddeee | 601 |
| 601267 | 2600 | 2615 | Exon 18 | TGTTTTAATTCAATCC | 43 | 7835 | 7850 | edkkdddddddddeee | 602 |
| 601268 | 2601 | 2616 | Exon 18 | CTGTTTTAATTCAATC | 44 | 7836 | 7851 | edkkdddddddddeee | 603 |
| 601269 | 2602 | 2617 | Exon 18 | GCTGTTTTAATTCAAT | 66 | 7837 | 7852 | edkkdddddddddeee | 604 |
| 601270 | 2603 | 2618 | Exon 18 | AGCTGTTTTAATTCAA | 47 | 7838 | 7853 | edkkdddddddddeee | 605 |
| 532917 | 2604 | 2623 | Exon 18 | GTCGCAGCTGTTTTAATT | 3 | 7839 | 7858 | eeeeeddddddddddeeee | 317 |
| | | | | CA | | | | e | |
| 601271 | 2604 | 2619 | Exon 18 | CAGCTGTTTTAATTCA | 26 | 7839 | 7854 | edkkdddddddddeee | 606 |
| 601272 | 2605 | 2620 | Exon 18 | GCAGCTGTTTTAATTC | 33 | 7840 | 7855 | edkkdddddddddeee | 607 |
| 601273 | 2606 | 2621 | Exon 18 | CGCAGCTGTTTTAATT | 34 | 7841 | 7856 | edkkdddddddddeee | 608 |
| 601274 | 2607 | 2622 | Exon 18 | TCGCAGCTGTTTTAAT | 39 | 7842 | 7857 | edkkdddddddddeee | 609 |
| 588860 | 2608 | 2623 | Exon 18 | GTCGCAGCTGTTTTAA | 72 | 7843 | 7858 | eekdddddddddddke | 610 |
| 601275 | 2608 | 2623 | Exon 18 | GTCGCAGCTGTTTTAA | 65 | 7843 | 7858 | edkkdddddddddeee | 610 |
| 601276 | 2609 | 2624 | Exon 18 | TGTCGCAGCTGTTTTA | 65 | 7844 | 7859 | edkkdddddddddeee | 611 |
| 601277 | 2610 | 2625 | Exon 18 | TTGTCGCAGCTGTTTT | 51 | 7845 | 7860 | edkkdddddddddeee | 612 |
| 601278 | 2611 | 2626 | Exon 18 | GTTGTCGCAGCTGTTT | 78 | 7846 | 7861 | edkkdddddddddeee | 613 |
| 601279 | 2612 | 2627 | Exon 18 | TGTTGTCGCAGCTGTT | 79 | 7847 | 7862 | edkkdddddddddeee | 614 |
| 601280 | 2613 | 2628 | Exon 18/ | TTGTTGTCGCAGCTGT | 70 | n/a | n/a | edkkdddddddddeee | 615 |
| | | | Repeat | | | | | | |
| 601281 | 2614 | 2629 | Exon 18/ | TTTGTTGTCGCAGCTG | 78 | n/a | n/a | edkkdddddddddeee | 616 |
| | | | Repeat | | | | | | |
| 601282 | 2615 | 2630 | Exon 18/ | TTTTGTTGTCGCAGCT | 68 | n/a | n/a | edkkdddddddddeee | 617 |
| | | | Repeat | | | | | | |
| 601283 | 2616 | 2631 | Exon 18/ | TTTTTGTTGTCGCAGC | 61 | n/a | n/a | edkkdddddddddeee | 618 |
| | | | Repeat | | | | | | |

TABLE 144
Inhibition of CFB mRNA by deoxy, MOE and (S)-cEt oligonucleotides targeting SEQ ID NO: 1
or SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | start | stop | Target | | inhi- | start | stop | | ID |
| NO | site | site | region | Sequence | bition | site | site | Motif | NO: |
| 601306 | 2573 | 2588 | Exon 18 | CCAGCAGGAAACCCCT | 22 | 7808 | 7823 | ekkdddddddddkeee | 575 |
| 601307 | 2574 | 2589 | Exon 18 | TCCAGCAGGAAACCCC | 22 | 7809 | 7824 | ekkdddddddddkeee | 576 |
| 601308 | 2575 | 2590 | Exon 18 | GTCCAGCAGGAAACCC | 33 | 7810 | 7825 | ekkdddddddddkeee | 577 |
| 601309 | 2576 | 2591 | Exon 18 | TGTCCAGCAGGAAACC | 33 | 7811 | 7826 | ekkdddddddddkeee | 578 |
| 601310 | 2577 | 2592 | Exon 18 | CTGTCCAGCAGGAAAC | 28 | 7812 | 7827 | ekkdddddddddkeee | 579 |
| 601311 | 2578 | 2593 | Exon 18 | CCTGTCCAGCAGGAAA | 33 | 7813 | 7828 | ekkdddddddddkeee | 580 |
| 601312 | 2579 | 2594 | Exon 18 | CCCTGTCCAGCAGGAA | 13 | 7814 | 7829 | ekkdddddddddkeee | 581 |
| 601313 | 2580 | 2595 | Exon 18 | CCCCTGTCCAGCAGGA | 32 | 7815 | 7830 | ekkdddddddddkeee | 582 |
| 601314 | 2581 | 2596 | Exon 18 | GCCCCTGTCCAGCAGG | 0 | 7816 | 7831 | ekkdddddddddkeee | 583 |
| 601315 | 2582 | 2597 | Exon 18 | CGCCCCTGTCCAGCAG | 36 | 7817 | 7832 | ekkdddddddddkeee | 584 |
| 601316 | 2583 | 2598 | Exon 18 | ACGCCCCTGTCCAGCA | 39 | 7818 | 7833 | ekkdddddddddkeee | 585 |
| 601317 | 2584 | 2599 | Exon 18 | CACGCCCCTGTCCAGC | 33 | 7819 | 7834 | ekkdddddddddkeee | 586 |
| 601356 | 2584 | 2599 | Exon 18 | CACGCCCCTGTCCAGC | 27 | 7819 | 7834 | kdkdddddddddeeee | 586 |
| 601318 | 2585 | 2600 | Exon 18 | CCACGCCCCTGTCCAG | 35 | 7820 | 7835 | ekkdddddddddkeee | 587 |
| 601357 | 2585 | 2600 | Exon 18 | CCACGCCCCTGTCCAG | 26 | 7820 | 7835 | kdkdddddddddeeee | 587 |
| 601319 | 2586 | 2601 | Exon 18 | CCCACGCCCCTGTCCA | 33 | 7821 | 7836 | ekkdddddddddkeee | 588 |
| 601358 | 2586 | 2601 | Exon 18 | CCCACGCCCCTGTCCA | 26 | 7821 | 7836 | kdkdddddddddeeee | 588 |
| 601320 | 2587 | 2602 | Exon 18 | TCCCACGCCCCTGTCC | 25 | 7822 | 7837 | ekkdddddddddkeee | 589 |
| 601359 | 2587 | 2602 | Exon 18 | TCCCACGCCCCTGTCC | 23 | 7822 | 7837 | kdkdddddddddeeee | 589 |
| 601321 | 2588 | 2603 | Exon 18 | ATCCCACGCCCCTGTC | 50 | 7823 | 7838 | ekkdddddddddkeee | 590 |
| 601360 | 2588 | 2603 | Exon 18 | ATCCCACGCCCCTGTC | 33 | 7823 | 7838 | kdkdddddddddeeee | 590 |
| 601322 | 2589 | 2604 | Exon 18 | AATCCCACGCCCCTGT | 52 | 7824 | 7839 | ekkdddddddddkeee | 591 |
| 601361 | 2589 | 2604 | Exon 18 | AATCCCACGCCCCTGT | 48 | 7824 | 7839 | kdkdddddddddeeee | 591 |
| 601323 | 2590 | 2605 | Exon 18 | CAATCCCACGCCCCTG | 67 | 7825 | 7840 | ekkdddddddddkeee | 592 |
| 601362 | 2590 | 2605 | Exon 18 | CAATCCCACGCCCCTG | 51 | 7825 | 7840 | kdkdddddddddeeee | 592 |
| 601324 | 2591 | 2606 | Exon 18 | TCAATCCCACGCCCCT | 42 | 7826 | 7841 | ekkdddddddddkeee | 593 |
| 601363 | 2591 | 2606 | Exon 18 | TCAATCCCACGCCCCT | 42 | 7826 | 7841 | kdkdddddddddeeee | 593 |
| 601325 | 2592 | 2607 | Exon 18 | TTCAATCCCACGCCCC | 52 | 7827 | 7842 | ekkdddddddddkeee | 594 |
| 601364 | 2592 | 2607 | Exon 18 | TTCAATCCCACGCCCC | 48 | 7827 | 7842 | kdkdddddddddeeee | 594 |
| 601326 | 2593 | 2608 | Exon 18 | ATTCAATCCCACGCCC | 27 | 7828 | 7843 | ekkdddddddddkeee | 595 |
| 601365 | 2593 | 2608 | Exon 18 | ATTCAATCCCACGCCC | 36 | 7828 | 7843 | kdkdddddddddeeee | 595 |
| 601327 | 2594 | 2609 | Exon 18 | AATTCAATCCCACGCC | 66 | 7829 | 7844 | ekkdddddddddkeee | 596 |
| 601366 | 2594 | 2609 | Exon 18 | AATTCAATCCCACGCC | 49 | 7829 | 7844 | kdkdddddddddeeee | 596 |
| 601328 | 2595 | 2610 | Exon 18 | TAATTCAATCCCACGC | 55 | 7830 | 7845 | ekkdddddddddkeee | 597 |
| 601367 | 2595 | 2610 | Exon 18 | TAATTCAATCCCACGC | 57 | 7830 | 7845 | kdkdddddddddeeee | 597 |
| 601329 | 2596 | 2611 | Exon 18 | TTAATTCAATCCCACG | 69 | 7831 | 7846 | ekkdddddddddkeee | 598 |
| 601368 | 2596 | 2611 | Exon 18 | TTAATTCAATCCCACG | 68 | 7831 | 7846 | kdkdddddddddeeee | 598 |
| 601330 | 2597 | 2612 | Exon 18 | TTTAATTCAATCCCAC | 58 | 7832 | 7847 | ekkdddddddddkeee | 599 |
| 601369 | 2597 | 2612 | Exon 18 | TTTAATTCAATCCCAC | 65 | 7832 | 7847 | kdkdddddddddeeee | 599 |
| 601331 | 2598 | 2613 | Exon 18 | TTTTAATTCAATCCCA | 45 | 7833 | 7848 | ekkdddddddddkeee | 600 |
| 601370 | 2598 | 2613 | Exon 18 | TTTTAATTCAATCCCA | 42 | 7833 | 7848 | kdkdddddddddeeee | 600 |
| 601332 | 2599 | 2614 | Exon 18 | GTTTTAATTCAATCCC | 84 | 7834 | 7849 | ekkdddddddddkeee | 601 |
| 601371 | 2599 | 2614 | Exon 18 | GTTTTAATTCAATCCC | 79 | 7834 | 7849 | kdkdddddddddeeee | 601 |
| 601333 | 2600 | 2615 | Exon 18 | TGTTTTAATTCAATCC | 61 | 7835 | 7850 | ekkdddddddddkeee | 602 |
| 601372 | 2600 | 2615 | Exon 18 | TGTTTTAATTCAATCC | 71 | 7835 | 7850 | kdkdddddddddeeee | 602 |
| 601334 | 2601 | 2616 | Exon 18 | CTGTTTTAATTCAATC | 61 | 7836 | 7851 | ekkdddddddddkeee | 603 |
| 601373 | 2601 | 2616 | Exon 18 | CTGTTTTAATTCAATC | 57 | 7836 | 7851 | kdkdddddddddeeee | 603 |
| 601335 | 2602 | 2617 | Exon 18 | GCTGTTTTAATTCAAT | 73 | 7837 | 7852 | ekkdddddddddkeee | 604 |
| 601374 | 2602 | 2617 | Exon 18 | GCTGTTTTAATTCAAT | 66 | 7837 | 7852 | kdkdddddddddeeee | 604 |
| 601336 | 2603 | 2618 | Exon 18 | AGCTGTTTTAATTCAA | 64 | 7838 | 7853 | ekkdddddddddkeee | 605 |
| 601375 | 2603 | 2618 | Exon 18 | AGCTGTTTTAATTCAA | 61 | 7838 | 7853 | kdkdddddddddeeee | 605 |
| 532917 | 2604 | 2623 | Exon 18 | GTCGCAGCTGTTTTAATT | 66 | 7839 | 7858 | eeeeedddddddddde | 317 |
| | | | | CA | | | | eeee | |
| 601337 | 2604 | 2619 | Exon 18 | CAGCTGTTTTAATTCA | 53 | 7839 | 7854 | ekkdddddddddkeee | 606 |
| 601376 | 2604 | 2619 | Exon 18 | CAGCTGTTTTAATTCA | 39 | 7839 | 7854 | kdkdddddddddeeee | 606 |
| 601338 | 2605 | 2620 | Exon 18 | GCAGCTGTTTTAATTC | 67 | 7840 | 7855 | ekkdddddddddkeee | 607 |
| 601377 | 2605 | 2620 | Exon 18 | GCAGCTGTTTTAATTC | 67 | 7840 | 7855 | kdkdddddddddeeee | 607 |
| 601339 | 2606 | 2621 | Exon 18 | CGCAGCTGTTTTAATT | 63 | 7841 | 7856 | ekkdddddddddkeee | 608 |
| 601378 | 2606 | 2621 | Exon 18 | CGCAGCTGTTTTAATT | 60 | 7841 | 7856 | kdkdddddddddeeee | 608 |
| 601340 | 2607 | 2622 | Exon 18 | TCGCAGCTGTTTTAAT | 40 | 7842 | 7857 | ekkdddddddddkeee | 609 |
| 601379 | 2607 | 2622 | Exon 18 | TCGCAGCTGTTTTAAT | 36 | 7842 | 7857 | kdkdddddddddeeee | 609 |
| 588860 | 2608 | 2623 | Exon 18 | GTCGCAGCTGTTTTAA | 84 | 7843 | 7858 | eekdddddddddddke | 610 |
| 601341 | 2608 | 2623 | Exon 18 | GTCGCAGCTGTTTTAA | 74 | 7843 | 7858 | ekkdddddddddkeee | 610 |
| 601380 | 2608 | 2623 | Exon 18 | GTCGCAGCTGTTTTAA | 78 | 7843 | 7858 | kdkdddddddddeeee | 610 |
| 601342 | 2609 | 2624 | Exon 18 | TGTCGCAGCTGTTTTA | 68 | 7844 | 7859 | ekkdddddddddkeee | 611 |
| 601381 | 2609 | 2624 | Exon 18 | TGTCGCAGCTGTTTTA | 66 | 7844 | 7859 | kdkdddddddddeeee | 611 |
| 601343 | 2610 | 2625 | Exon 18 | TTGTCGCAGCTGTTTT | 71 | 7845 | 7860 | ekkdddddddddkeee | 612 |
| 601382 | 2610 | 2625 | Exon 18 | TTGTCGCAGCTGTTTT | 84 | 7845 | 7860 | kdkdddddddddeeee | 612 |
| 601344 | 2611 | 2626 | Exon 18 | GTTGTCGCAGCTGTTT | 87 | 7846 | 7861 | ekkdddddddddkeee | 613 |
| 601383 | 2611 | 2626 | Exon 18 | GTTGTCGCAGCTGTTT | 85 | 7846 | 7861 | kdkdddddddddeeee | 613 |
| 601345 | 2612 | 2627 | Exon 18 | TGTTGTCGCAGCTGTT | 82 | 7847 | 7862 | ekkdddddddddkeee | 614 |
| 601384 | 2612 | 2627 | Exon 18 | TGTTGTCGCAGCTGTT | 79 | 7847 | 7862 | kdkdddddddddeeee | 614 |
| 601346 | 2613 | 2628 | Exon 18/ | TTGTTGTCGCAGCTGT | 73 | n/a | n/a | ekkdddddddddkeee | 615 |
| | | | Repeat | | | | | | |
| 601385 | 2613 | 2628 | Exon 18/ | TTGTTGTCGCAGCTGT | 84 | n/a | n/a | kdkdddddddddeeee | 615 |
| | | | Repeat | | | | | | |
| 601347 | 2614 | 2629 | Exon 18/ | TTTGTTGTCGCAGCTG | 70 | n/a | n/a | ekkdddddddddkeee | 616 |
| | | | Repeat | | | | | | |
| 601386 | 2614 | 2629 | Exon 18/ | TTTGTTGTCGCAGCTG | 71 | n/a | n/a | kdkdddddddddeeee | 616 |
| | | | Repeat | | | | | | |
| 601348 | 2615 | 2630 | Exon 18/ | TTTTGTTGTCGCAGCT | 71 | n/a | n/a | ekkdddddddddkeee | 617 |
| | | | Repeat | | | | | | |
| 601387 | 2615 | 2630 | Exon 18/ | TTTTGTTGTCGCAGCT | 76 | n/a | n/a | kdkdddddddddeeee | 617 |
| | | | Repeat | | | | | | |
| 601349 | 2616 | 2631 | Exon 18/ | TTTTTGTTGTCGCAGC | 71 | n/a | n/a | ekkdddddddddkeee | 618 |
| | | | Repeat | | | | | | |
| 601388 | 2616 | 2631 | Exon 18/ | TTTTTGTTGTCGCAGC | 67 | n/a | n/a | kdkdddddddddeeee | 618 |
| | | | Repeat | | | | | | |

TABLE 145
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | start | stop | Target | | inhi- | start | stop | | ID |
| NO | site | site | region | Sequence | bition | site | site | Motif | NO: |
| 599357 | 2582 | 2600 | Exon 18 | CCACGCCCCTGTCCAGCAG | 26 | 7817 | 7835 | 5-9-5 | 708 |
| 599358 | 2583 | 2601 | Exon 18 | CCCACGCCCCTGTCCAGCA | 22 | 7818 | 7836 | 5-9-5 | 709 |
| 599359 | 2584 | 2602 | Exon 18 | TCCCACGCCCCTGTCCAGC | 13 | 7819 | 7837 | 5-9-5 | 710 |
| 599360 | 2585 | 2603 | Exon 18 | ATCCCACGCCCCTGTCCAG | 7 | 7820 | 7838 | 5-9-5 | 711 |
| 599361 | 2586 | 2604 | Exon 18 | AATCCCACGCCCCTGTCCA | 11 | 7821 | 7839 | 5-9-5 | 712 |
| 599362 | 2587 | 2605 | Exon 18 | CAATCCCACGCCCCTGTCC | 14 | 7822 | 7840 | 5-9-5 | 713 |
| 599363 | 2588 | 2606 | Exon 18 | TCAATCCCACGCCCCTGTC | 17 | 7823 | 7841 | 5-9-5 | 714 |
| 599364 | 2589 | 2607 | Exon 18 | TTCAATCCCACGCCCCTGT | 20 | 7824 | 7842 | 5-9-5 | 715 |
| 599365 | 2590 | 2608 | Exon 18 | ATTCAATCCCACGCCCCTG | 22 | 7825 | 7843 | 5-9-5 | 716 |
| 599366 | 2591 | 2609 | Exon 18 | AATTCAATCCCACGCCCCT | 13 | 7826 | 7844 | 5-9-5 | 717 |
| 599367 | 2592 | 2610 | Exon 18 | TAATTCAATCCCACGCCCC | 11 | 7827 | 7845 | 5-9-5 | 718 |
| 599368 | 2593 | 2611 | Exon 18 | TTAATTCAATCCCACGCCC | 10 | 7828 | 7846 | 5-9-5 | 719 |
| 599369 | 2594 | 2612 | Exon 18 | TTTAATTCAATCCCACGCC | 19 | 7829 | 7847 | 5-9-5 | 720 |
| 599370 | 2595 | 2613 | Exon 18 | TTTTAATTCAATCCCACGC | 23 | 7830 | 7848 | 5-9-5 | 721 |
| 599371 | 2596 | 2614 | Exon 18 | GTTTTAATTCAATCCCACG | 4 | 7831 | 7849 | 5-9-5 | 722 |
| 599372 | 2597 | 2615 | Exon 18 | TGTTTTAATTCAATCCCAC | 16 | 7832 | 7850 | 5-9-5 | 723 |
| 599373 | 2598 | 2616 | Exon 18 | CTGTTTTAATTCAATCCCA | 3 | 7833 | 7851 | 5-9-5 | 724 |
| 599374 | 2599 | 2617 | Exon 18 | GCTGTTTTAATTCAATCCC | 10 | 7834 | 7852 | 5-9-5 | 725 |
| 599375 | 2600 | 2618 | Exon 18 | AGCTGTTTTAATTCAATCC | 17 | 7835 | 7853 | 5-9-5 | 726 |
| 599376 | 2601 | 2619 | Exon 18 | CAGCTGTTTTAATTCAATC | 18 | 7836 | 7854 | 5-9-5 | 727 |
| 599377 | 2602 | 2620 | Exon 18 | GCAGCTGTTTTAATTCAAT | 22 | 7837 | 7855 | 5-9-5 | 728 |
| 599378 | 2603 | 2621 | Exon 18 | CGCAGCTGTTTTAATTCAA | 11 | 7838 | 7856 | 5-9-5 | 729 |
| 599511 | 2552 | 2571 | Exon 18 | ATAGAAAACCCAAATCCTCA | 7 | 7787 | 7806 | 6-8-6 | 410 |
| 599389 | 2553 | 2572 | Exon 18 | TATAGAAAACCCAAATCCTC | 22 | 7788 | 7807 | 6-8-6 | 411 |
| 599390 | 2554 | 2573 | Exon 18 | TTATAGAAAACCCAAATCCT | 21 | 7789 | 7808 | 6-8-6 | 412 |
| 599391 | 2555 | 2574 | Exon 18 | CTTATAGAAAACCCAAATCC | 27 | 7790 | 7809 | 6-8-6 | 413 |
| 599392 | 2556 | 2575 | Exon 18 | CCTTATAGAAAACCCAAATC | 30 | 7791 | 7810 | 6-8-6 | 414 |
| 599393 | 2557 | 2576 | Exon 18 | CCCTTATAGAAAACCCAAAT | 30 | 7792 | 7811 | 6-8-6 | 415 |
| 599394 | 2558 | 2577 | Exon 18 | CCCCTTATAGAAAACCCAAA | 28 | 7793 | 7812 | 6-8-6 | 416 |
| 599395 | 2559 | 2578 | Exon 18 | ACCCCTTATAGAAAACCCAA | 23 | 7794 | 7813 | 6-8-6 | 417 |
| 599396 | 2560 | 2579 | Exon 18 | AACCCCTTATAGAAAACCCA | 53 | 7795 | 7814 | 6-8-6 | 418 |
| 599397 | 2561 | 2580 | Exon 18 | AAACCCCTTATAGAAAACCC | 33 | 7796 | 7815 | 6-8-6 | 419 |
| 599398 | 2562 | 2581 | Exon 18 | GAAACCCCTTATAGAAAACC | 58 | 7797 | 7816 | 6-8-6 | 420 |
| 599399 | 2563 | 2582 | Exon 18 | GGAAACCCCTTATAGAAAAC | 23 | 7798 | 7817 | 6-8-6 | 421 |
| 599400 | 2564 | 2583 | Exon 18 | AGGAAACCCCTTATAGAAAA | 54 | 7799 | 7818 | 6-8-6 | 422 |
| 599401 | 2565 | 2584 | Exon 18 | CAGGAAACCCCTTATAGAAA | 30 | 7800 | 7819 | 6-8-6 | 423 |
| 599402 | 2566 | 2585 | Exon 18 | GCAGGAAACCCCTTATAGAA | 25 | 7801 | 7820 | 6-8-6 | 424 |
| 599403 | 2567 | 2586 | Exon 18 | AGCAGGAAACCCCTTATAGA | 17 | 7802 | 7821 | 6-8-6 | 425 |
| 599404 | 2568 | 2587 | Exon 18 | CAGCAGGAAACCCCTTATAG | 20 | 7803 | 7822 | 6-8-6 | 426 |
| 599405 | 2569 | 2588 | Exon 18 | CCAGCAGGAAACCCCTTATA | 12 | 7804 | 7823 | 6-8-6 | 427 |
| 599406 | 2570 | 2589 | Exon 18 | TCCAGCAGGAAACCCCTTAT | 51 | 7805 | 7824 | 6-8-6 | 428 |
| 599407 | 2571 | 2590 | Exon 18 | GTCCAGCAGGAAACCCCTTA | 39 | 7806 | 7825 | 6-8-6 | 237 |
| 599408 | 2572 | 2591 | Exon 18 | TGTCCAGCAGGAAACCCCTT | 53 | 7807 | 7826 | 6-8-6 | 429 |
| 599409 | 2573 | 2592 | Exon 18 | CTGTCCAGCAGGAAACCCCT | 65 | 7808 | 7827 | 6-8-6 | 430 |
| 599410 | 2574 | 2593 | Exon 18 | CCTGTCCAGCAGGAAACCCC | 56 | 7809 | 7828 | 6-8-6 | 431 |
| 599411 | 2575 | 2594 | Exon 18 | CCCTGTCCAGCAGGAAACCC | 60 | 7810 | 7829 | 6-8-6 | 432 |
| 599412 | 2576 | 2595 | Exon 18 | CCCCTGTCCAGCAGGAAACC | 61 | 7811 | 7830 | 6-8-6 | 433 |
| 599413 | 2577 | 2596 | Exon 18 | GCCCCTGTCCAGCAGGAAAC | 40 | 7812 | 7831 | 6-8-6 | 238 |
| 599414 | 2578 | 2597 | Exon 18 | CGCCCCTGTCCAGCAGGAAA | 41 | 7813 | 7832 | 6-8-6 | 434 |
| 599415 | 2579 | 2598 | Exon 18 | ACGCCCCTGTCCAGCAGGAA | 37 | 7814 | 7833 | 6-8-6 | 435 |
| 599416 | 2580 | 2599 | Exon 18 | CACGCCCCTGTCCAGCAGGA | 54 | 7815 | 7834 | 6-8-6 | 436 |
| 599417 | 2581 | 2600 | Exon 18 | CCACGCCCCTGTCCAGCAGG | 36 | 7816 | 7835 | 6-8-6 | 437 |
| 599418 | 2582 | 2601 | Exon 18 | CCCACGCCCCTGTCCAGCAG | 53 | 7817 | 7836 | 6-8-6 | 438 |
| 599419 | 2583 | 2602 | Exon 18 | TCCCACGCCCCTGTCCAGCA | 54 | 7818 | 7837 | 6-8-6 | 439 |
| 599420 | 2584 | 2603 | Exon 18 | ATCCCACGCCCCTGTCCAGC | 50 | 7819 | 7838 | 6-8-6 | 440 |
| 599421 | 2585 | 2604 | Exon 18 | AATCCCACGCCCCTGTCCAG | 48 | 7820 | 7839 | 6-8-6 | 441 |
| 599422 | 2586 | 2605 | Exon 18 | CAATCCCACGCCCCTGTCCA | 55 | 7821 | 7840 | 6-8-6 | 442 |
| 599423 | 2587 | 2606 | Exon 18 | TCAATCCCACGCCCCTGTCC | 75 | 7822 | 7841 | 6-8-6 | 443 |
| 599424 | 2588 | 2607 | Exon 18 | TTCAATCCCACGCCCCTGTC | 69 | 7823 | 7842 | 6-8-6 | 444 |
| 599425 | 2589 | 2608 | Exon 18 | ATTCAATCCCACGCCCCTGT | 77 | 7824 | 7843 | 6-8-6 | 445 |
| 599426 | 2590 | 2609 | Exon 18 | AATTCAATCCCACGCCCCTG | 60 | 7825 | 7844 | 6-8-6 | 446 |
| 599427 | 2591 | 2610 | Exon 18 | TAATTCAATCCCACGCCCCT | 72 | 7826 | 7845 | 6-8-6 | 447 |
| 599428 | 2592 | 2611 | Exon 18 | TTAATTCAATCCCACGCCCC | 81 | 7827 | 7846 | 6-8-6 | 448 |
| 599429 | 2593 | 2612 | Exon 18 | TTTAATTCAATCCCACGCCC | 68 | 7828 | 7847 | 6-8-6 | 449 |
| 599430 | 2594 | 2613 | Exon 18 | TTTTAATTCAATCCCACGCC | 58 | 7829 | 7848 | 6-8-6 | 450 |
| 599431 | 2595 | 2614 | Exon 18 | GTTTTAATTCAATCCCACGC | 70 | 7830 | 7849 | 6-8-6 | 451 |
| 599432 | 2596 | 2615 | Exon 18 | TGTTTTAATTCAATCCCACG | 85 | 7831 | 7850 | 6-8-6 | 452 |
| 532917 | 2604 | 2623 | Exon 18 | GTCGCAGCTGTTTTAATTCA | 85 | 7839 | 7858 | 5-10-5 | 317 |
| 599379 | 2604 | 2622 | Exon 18 | TCGCAGCTGTTTTAATTCA | 73 | 7839 | 7857 | 5-9-5 | 730 |
| 599380 | 2605 | 2623 | Exon 18 | GTCGCAGCTGTTTTAATTC | 77 | 7840 | 7858 | 5-9-5 | 731 |
| 599381 | 2606 | 2624 | Exon 18 | TGTCGCAGCTGTTTTAATT | 69 | 7841 | 7859 | 5-9-5 | 732 |
| 599382 | 2607 | 2625 | Exon 18 | TTGTCGCAGCTGTTTTAAT | 58 | 7842 | 7860 | 5-9-5 | 733 |
| 599383 | 2608 | 2626 | Exon 18 | GTTGTCGCAGCTGTTTTAA | 52 | 7843 | 7861 | 5-9-5 | 734 |
| 599384 | 2609 | 2627 | Exon 18 | TGTTGTCGCAGCTGTTTTA | 63 | 7844 | 7862 | 5-9-5 | 735 |
| 599385 | 2610 | 2628 | Exon 18/ | TTGTTGTCGCAGCTGTTTT | 53 | n/a | n/a | 5-9-5 | 736 |
| | | | Repeat | | | | | | |
| 599386 | 2611 | 2629 | Exon 18/ | TTTGTTGTCGCAGCTGTTT | 63 | n/a | n/a | 5-9-5 | 737 |
| | | | Repeat | | | | | | |
| 599387 | 2612 | 2630 | Exon 18/ | TTTTGTTGTCGCAGCTGTT | 64 | n/a | n/a | 5-9-5 | 438 |
| | | | Repeat | | | | | | |
| 599388 | 2613 | 2631 | Exon 18/ | TTTTTGTTGTCGCAGCTGT | 66 | n/a | n/a | 5-9-5 | 739 |
| | | | Repeat | | | | | | |

TABLE 146
Inhibition of CFB mRNA by MOE gapmers targeting SEQ ID NO: 1 or
SEQ ID NO: 2

| | SEQ ID | SEQ ID | | | | SEQ ID | SEQ ID | | |
|---|---|---|---|---|---|---|---|---|---|
| | NO: 1 | NO: 1 | | | % | NO: 2 | NO: 2 | | SEQ |
| ISIS | start | stop | Target | | inhi- | start | stop | | ID |
| NO | site | site | region | Sequence | bition | site | site | Motif | NO: |
| 599213 | 2553 | 2570 | Exon 18 | TAGAAAACCCAAATCCTC | 0 | 7788 | 7805 | 3-10-5 | 785 |
| 599214 | 2554 | 2571 | Exon 18 | ATAGAAAACCCAAATCCT | 0 | 7789 | 7806 | 3-10-5 | 786 |
| 599215 | 2555 | 2572 | Exon 18 | TATAGAAAACCCAAATCC | 36 | 7790 | 7807 | 3-10-5 | 787 |
| 599216 | 2556 | 2573 | Exon 18 | TTATAGAAAACCCAAATC | 8 | 7791 | 7808 | 3-10-5 | 788 |
| 599217 | 2557 | 2574 | Exon 18 | CTTATAGAAAACCCAAAT | 5 | 7792 | 7809 | 3-10-5 | 789 |
| 599218 | 2558 | 2575 | Exon 18 | CCTTATAGAAAACCCAAA | 0 | 7793 | 7810 | 3-10-5 | 790 |
| 599219 | 2559 | 2576 | Exon 18 | CCCTTATAGAAAACCCAA | 8 | 7794 | 7811 | 3-10-5 | 791 |
| 599220 | 2560 | 2577 | Exon 18 | CCCCTTATAGAAAACCCA | 0 | 7795 | 7812 | 3-10-5 | 740 |
| 599221 | 2561 | 2578 | Exon 18 | ACCCCTTATAGAAAACCC | 54 | 7796 | 7813 | 3-10-5 | 741 |
| 599222 | 2562 | 2579 | Exon 18 | AACCCCTTATAGAAAACC | 3 | 7797 | 7814 | 3-10-5 | 742 |
| 599223 | 2563 | 2580 | Exon 18 | AAACCCCTTATAGAAAAC | 0 | 7798 | 7815 | 3-10-5 | 743 |
| 599224 | 2564 | 2581 | Exon 18 | GAAACCCCTTATAGAAAA | 0 | 7799 | 7816 | 3-10-5 | 744 |
| 599225 | 2566 | 2583 | Exon 18 | AGGAAACCCCTTATAGAA | 60 | 7801 | 7818 | 3-10-5 | 745 |
| 599226 | 2567 | 2584 | Exon 18 | CAGGAAACCCCTTATAGA | 0 | 7802 | 7819 | 3-10-5 | 746 |
| 599227 | 2568 | 2585 | Exon 18 | GCAGGAAACCCCTTATAG | 37 | 7803 | 7820 | 3-10-5 | 747 |
| 599228 | 2569 | 2586 | Exon 18 | AGCAGGAAACCCCTTATA | 0 | 7804 | 7821 | 3-10-5 | 748 |
| 599229 | 2570 | 2587 | Exon 18 | CAGCAGGAAACCCCTTAT | 39 | 7805 | 7822 | 3-10-5 | 749 |
| 599230 | 2571 | 2588 | Exon 18 | CCAGCAGGAAACCCCTTA | 10 | 7806 | 7823 | 3-10-5 | 750 |
| 599231 | 2572 | 2589 | Exon 18 | TCCAGCAGGAAACCCCTT | 16 | 7807 | 7824 | 3-10-5 | 751 |
| 599232 | 2573 | 2590 | Exon 18 | GTCCAGCAGGAAACCCCT | 9 | 7808 | 7825 | 3-10-5 | 752 |
| 599233 | 2574 | 2591 | Exon 18 | TGTCCAGCAGGAAACCCC | 44 | 7809 | 7826 | 3-10-5 | 753 |
| 599234 | 2575 | 2592 | Exon 18 | CTGTCCAGCAGGAAACCC | 14 | 7810 | 7827 | 3-10-5 | 754 |
| 599235 | 2576 | 2593 | Exon 18 | CCTGTCCAGCAGGAAACC | 0 | 7811 | 7828 | 3-10-5 | 755 |
| 599236 | 2577 | 2594 | Exon 18 | CCCTGTCCAGCAGGAAAC | 43 | 7812 | 7829 | 3-10-5 | 756 |
| 599237 | 2578 | 2595 | Exon 18 | CCCCTGTCCAGCAGGAAA | 0 | 7813 | 7830 | 3-10-5 | 757 |
| 599238 | 2580 | 2597 | Exon 18 | CGCCCCTGTCCAGCAGGA | 9 | 7815 | 7832 | 3-10-5 | 758 |
| 599239 | 2581 | 2598 | Exon 18 | ACGCCCCTGTCCAGCAGG | 36 | 7816 | 7833 | 3-10-5 | 759 |
| 599240 | 2582 | 2599 | Exon 18 | CACGCCCCTGTCCAGCAG | 11 | 7817 | 7834 | 3-10-5 | 760 |
| 599241 | 2583 | 2600 | Exon 18 | CCACGCCCCTGTCCAGCA | 51 | 7818 | 7835 | 3-10-5 | 761 |
| 599242 | 2584 | 2601 | Exon 18 | CCCACGCCCCTGTCCAGC | 7 | 7819 | 7836 | 3-10-5 | 762 |
| 599243 | 2585 | 2602 | Exon 18 | TCCCACGCCCCTGTCCAG | 47 | 7820 | 7837 | 3-10-5 | 763 |
| 599244 | 2586 | 2603 | Exon 18 | ATCCCACGCCCCTGTCCA | 37 | 7821 | 7838 | 3-10-5 | 764 |
| 599245 | 2587 | 2604 | Exon 18 | AATCCCACGCCCCTGTCC | 35 | 7822 | 7839 | 3-10-5 | 765 |
| 599246 | 2588 | 2605 | Exon 18 | CAATCCCACGCCCCTGTC | 21 | 7823 | 7840 | 3-10-5 | 766 |
| 599247 | 2589 | 2606 | Exon 18 | TCAATCCCACGCCCCTGT | 61 | 7824 | 7841 | 3-10-5 | 767 |
| 599248 | 2590 | 2607 | Exon 18 | TTCAATCCCACGCCCCTG | 51 | 7825 | 7842 | 3-10-5 | 768 |
| 599249 | 2591 | 2608 | Exon 18 | ATTCAATCCCACGCCCCT | 58 | 7826 | 7843 | 3-10-5 | 769 |
| 599250 | 2592 | 2609 | Exon 18 | AATTCAATCCCACGCCCC | 49 | 7827 | 7844 | 3-10-5 | 770 |
| 599251 | 2593 | 2610 | Exon 18 | TAATTCAATCCCACGCCC | 46 | 7828 | 7845 | 3-10-5 | 771 |
| 599252 | 2594 | 2611 | Exon 18 | TTAATTCAATCCCACGCC | 32 | 7829 | 7846 | 3-10-5 | 772 |
| 599253 | 2595 | 2612 | Exon 18 | TTTAATTCAATCCCACGC | 23 | 7830 | 7847 | 3-10-5 | 773 |
| 599254 | 2596 | 2613 | Exon 18 | TTTTAATTCAATCCCACG | 0 | 7831 | 7848 | 3-10-5 | 774 |
| 599255 | 2597 | 2614 | Exon 18 | GTTTTAATTCAATCCCAC | 61 | 7832 | 7849 | 3-10-5 | 775 |
| 599256 | 2598 | 2615 | Exon 18 | TGTTTTAATTCAATCCCA | 64 | 7833 | 7850 | 3-10-5 | 776 |
| 599257 | 2599 | 2616 | Exon 18 | CTGTTTTAATTCAATCCC | 66 | 7834 | 7851 | 3-10-5 | 777 |
| 599258 | 2600 | 2617 | Exon 18 | GCTGTTTTAATTCAATCC | 59 | 7835 | 7852 | 3-10-5 | 778 |
| 599259 | 2601 | 2618 | Exon 18 | AGCTGTTTTAATTCAATC | 40 | 7836 | 7853 | 3-10-5 | 779 |
| 599260 | 2602 | 2619 | Exon 18 | CAGCTGTTTTAATTCAAT | 38 | 7837 | 7854 | 3-10-5 | 780 |
| 599261 | 2603 | 2620 | Exon 18 | GCAGCTGTTTTAATTCAA | 54 | 7838 | 7855 | 3-10-5 | 781 |
| 599509 | 2552 | 2570 | Exon 18 | TAGAAAACCCAAATCCTCA | 54 | 7787 | 7805 | 6-7-6 | 681 |
| 599273 | 2553 | 2571 | Exon 18 | ATAGAAAACCCAAATCCTC | 0 | 7788 | 7806 | 6-7-6 | 682 |
| 599274 | 2554 | 2572 | Exon 18 | TATAGAAAACCCAAATCCT | 57 | 7789 | 7807 | 6-7-6 | 683 |
| 599275 | 2556 | 2574 | Exon 18 | CTTATAGAAAACCCAAATC | 0 | 7791 | 7809 | 6-7-6 | 684 |
| 599276 | 2557 | 2575 | Exon 18 | CCTTATAGAAAACCCAAAT | 44 | 7792 | 7810 | 6-7-6 | 685 |
| 599277 | 2558 | 2576 | Exon 18 | CCCTTATAGAAAACCCAAA | 0 | 7793 | 7811 | 6-7-6 | 686 |
| 599278 | 2559 | 2577 | Exon 18 | CCCCTTATAGAAAACCCAA | 0 | 7794 | 7812 | 6-7-6 | 687 |
| 599279 | 2560 | 2578 | Exon 18 | ACCCCTTATAGAAAACCCA | 20 | 7795 | 7813 | 6-7-6 | 688 |
| 599280 | 2561 | 2579 | Exon 18 | AACCCCTTATAGAAAACCC | 70 | 7796 | 7814 | 6-7-6 | 689 |
| 532917 | 2604 | 2623 | Exon 18 | GTCGCAGCTGTTTTAATTCA | 85 | 7839 | 7858 | 5-10-5 | 317 |
| 599262 | 2604 | 2621 | Exon 18 | CGCAGCTGTTTTAATTCA | 49 | 7839 | 7856 | 3-10-5 | 782 |
| 599263 | 2605 | 2622 | Exon 18 | TCGCAGCTGTTTTAATTC | 49 | 7840 | 7857 | 3-10-5 | 783 |
| 599264 | 2606 | 2623 | Exon 18 | GTCGCAGCTGTTTTAATT | 62 | 7841 | 7858 | 3-10-5 | 784 |
| 599265 | 2607 | 2624 | Exon 18 | TGTCGCAGCTGTTTTAAT | 63 | 7842 | 7859 | 3-10-5 | 792 |
| 599266 | 2608 | 2625 | Exon 18 | TTGTCGCAGCTGTTTTAA | 41 | 7843 | 7860 | 3-10-5 | 793 |
| 599267 | 2609 | 2626 | Exon 18 | GTTGTCGCAGCTGTTTTA | 52 | 7844 | 7861 | 3-10-5 | 794 |
| 599268 | 2610 | 2627 | Exon 18 | TGTTGTCGCAGCTGTTTT | 51 | 7845 | 7862 | 3-10-5 | 795 |
| 599269 | 2611 | 2628 | Exon 18/ | TTGTTGTCGCAGCTGTTT | 58 | n/a | n/a | 3-10-5 | 796 |
| | | | Repeat | | | | | | |
| 599270 | 2612 | 2629 | Exon 18/ | TTTGTTGTCGCAGCTGTT | 69 | n/a | n/a | 3-10-5 | 797 |
| | | | Repeat | | | | | | |
| 599271 | 2613 | 2630 | Exon 18/ | TTTTGTTGTCGCAGCTGT | 69 | n/a | n/a | 3-10-5 | 798 |
| | | | Repeat | | | | | | |
| 599272 | 2614 | 2631 | Exon 18/ | TTTTTGTTGTCGCAGCTG | 72 | n/a | n/a | 3-10-5 | 799 |
| | | | Repeat | | | | | | |
| 599205 | 2607 | 2624 | Exon 18 | TGTCGCAGCTGTTTTAAT | 54 | 7842 | 7859 | 5-8-5 | 792 |
| 599206 | 2608 | 2625 | Exon 18 | TTGTCGCAGCTGTTTTAA | 62 | 7843 | 7860 | 5-8-5 | 793 |
| 599207 | 2609 | 2626 | Exon 18 | GTTGTCGCAGCTGTTTTA | 62 | 7844 | 7861 | 5-8-5 | 794 |
| 599208 | 2610 | 2627 | Exon 18 | TGTTGTCGCAGCTGTTTT | 66 | 7845 | 7862 | 5-8-5 | 795 |
| 599209 | 2611 | 2628 | Exon 18/ | TTGTTGTCGCAGCTGTTT | 60 | n/a | n/a | 5-8-5 | 796 |
| | | | Repeat | | | | | | |
| 599210 | 2612 | 2629 | Exon 18/ | TTTGTTGTCGCAGCTGTT | 62 | n/a | n/a | 5-8-5 | 797 |
| | | | Repeat | | | | | | |
| 599211 | 2613 | 2630 | Exon 18/ | TTTTGTTGTCGCAGCTGT | 65 | n/a | n/a | 5-8-5 | 798 |
| | | | Repeat | | | | | | |
| 599212 | 2614 | 2631 | Exon 18/ | TTTTTGTTGTCGCAGCTG | 67 | n/a | n/a | 5-8-5 | 799 |
| | | | Repeat | | | | | | |

TABLE 147
Inhibition of CFB mRNA by 5-10-5 MOE gapmers targeting SEQ ID NO: 1 or
SEQ ID NO: 2

| | SEQ ID | SEQ ID | | | | SEQ ID | SEQ ID | |
|---|---|---|---|---|---|---|---|---|
| | NO: 1 | NO: 1 | | | % | NO: 2 | NO: 2 | SEQ |
| ISIS | start | stop | Target | | inhi- | start | stop | ID |
| NO | site | site | region | Sequence | bition | site | site | NO: |
| 588570 | 150 | 169 | Exon 1 | TGGTCACATTCCCTTCCCCT | 72 | 1871 | 1890 | 396 |
| 588571 | 152 | 171 | Exon 1 | CCTGGTCACATTCCCTTCCC | 80 | 1873 | 1892 | 397 |
| 532614 | 154 | 173 | Exon 1 | GACCTGGTCACATTCCCTTC | 65 | 1875 | 1894 | 12 |
| 588572 | 156 | 175 | Exon 1 | TAGACCTGGTCACATTCCCT | 74 | 1877 | 1896 | 398 |
| 588573 | 158 | 177 | Exon 1 | CCTAGACCTGGTCACATTCC | 72 | 1879 | 1898 | 399 |
| 588566 | 2189 | 2208 | Exon 15 | CCTTCCGAGTCAGCTTTTTC | 66 | 6977 | 6996 | 400 |
| 588567 | 2191 | 2210 | Exon 15 | CTCCTTCCGAGTCAGCTTTT | 66 | 6979 | 6998 | 401 |
| 532770 | 2193 | 2212 | Exon 15 | ACCTCCTTCCGAGTCAGCTT | 64 | 6981 | 7000 | 198 |
| 588568 | 2195 | 2214 | Exon 15 | AGACCTCCTTCCGAGTCAGC | 78 | 6983 | 7002 | 402 |
| 588569 | 2197 | 2216 | Exon 15 | GTAGACCTCCTTCCGAGTCA | 74 | 6985 | 7004 | 403 |
| 588574 | 2453 | 2472 | Exon 18 | TTTGCCGCTTCTGGTTTTTG | 71 | 7688 | 7707 | 404 |
| 588575 | 2455 | 2474 | Exon 18 | CTTTTGCCGCTTCTGGTTTT | 72 | 7690 | 7709 | 405 |
| 532800 | 2457 | 2476 | Exon 18 | TGCTTTTGCCGCTTCTGGTT | 71 | 7692 | 7711 | 228 |
| 588576 | 2459 | 2478 | Exon 18 | CCTGCTTTTGCCGCTTCTGG | 59 | 7694 | 7713 | 406 |
| 588577 | 2461 | 2480 | Exon 18 | TACCTGCTTTTGCCGCTTCT | 76 | 7696 | 7715 | 407 |
| 516350 | 2550 | 2569 | Exon 18 | AGAAAACCCAAATCCTCATC | 58 | 7785 | 7804 | 408 |
| 588509 | 2551 | 2570 | Exon 18 | TAGAAAACCCAAATCCTCAT | 6 | 7786 | 7805 | 409 |
| 588510 | 2552 | 2571 | Exon 18 | ATAGAAAACCCAAATCCTCA | 10 | 7787 | 7806 | 410 |
| 588511 | 2553 | 2572 | Exon 18 | TATAGAAAACCCAAATCCTC | 9 | 7788 | 7807 | 411 |
| 588512 | 2554 | 2573 | Exon 18 | TTATAGAAAACCCAAATCCT | 80 | 7789 | 7808 | 412 |
| 588513 | 2555 | 2574 | Exon 18 | CTTATAGAAAACCCAAATCC | 70 | 7790 | 7809 | 413 |
| 588514 | 2556 | 2575 | Exon 18 | CCTTATAGAAAACCCAAATC | 71 | 7791 | 7810 | 414 |
| 588515 | 2557 | 2576 | Exon 18 | CCCTTATAGAAAACCCAAAT | 78 | 7792 | 7811 | 415 |
| 588516 | 2558 | 2577 | Exon 18 | CCCCTTATAGAAAACCCAAA | 72 | 7793 | 7812 | 416 |
| 588517 | 2559 | 2578 | Exon 18 | ACCCCTTATAGAAAACCCAA | 80 | 7794 | 7813 | 417 |
| 588518 | 2560 | 2579 | Exon 18 | AACCCCTTATAGAAAACCCA | 80 | 7795 | 7814 | 418 |
| 588519 | 2561 | 2580 | Exon 18 | AAACCCCTTATAGAAAACCC | 62 | 7796 | 7815 | 419 |
| 588520 | 2562 | 2581 | Exon 18 | GAAACCCCTTATAGAAAACC | 59 | 7797 | 7816 | 420 |
| 588521 | 2563 | 2582 | Exon 18 | GGAAACCCCTTATAGAAAAC | 40 | 7798 | 7817 | 421 |
| 588522 | 2564 | 2583 | Exon 18 | AGGAAACCCCTTATAGAAAA | 66 | 7799 | 7818 | 422 |
| 588523 | 2565 | 2584 | Exon 18 | CAGGAAACCCCTTATAGAAA | 63 | 7800 | 7819 | 423 |
| 588524 | 2566 | 2585 | Exon 18 | GCAGGAAACCCCTTATAGAA | 70 | 7801 | 7820 | 424 |
| 588525 | 2567 | 2586 | Exon 18 | AGCAGGAAACCCCTTATAGA | 67 | 7802 | 7821 | 425 |
| 588526 | 2568 | 2587 | Exon 18 | CAGCAGGAAACCCCTTATAG | 0 | 7803 | 7822 | 426 |
| 588527 | 2569 | 2588 | Exon 18 | CCAGCAGGAAACCCCTTATA | 11 | 7804 | 7823 | 427 |
| 588528 | 2570 | 2589 | Exon 18 | TCCAGCAGGAAACCCCTTAT | 15 | 7805 | 7824 | 428 |
| 532809 | 2571 | 2590 | Exon 18 | GTCCAGCAGGAAACCCCTTA | 75 | 7806 | 7825 | 237 |
| 588529 | 2572 | 2591 | Exon 18 | TGTCCAGCAGGAAACCCCTT | 16 | 7807 | 7826 | 429 |
| 588530 | 2573 | 2592 | Exon 18 | CTGTCCAGCAGGAAACCCCT | 16 | 7808 | 7827 | 430 |
| 588531 | 2574 | 2593 | Exon 18 | CCTGTCCAGCAGGAAACCCC | 19 | 7809 | 7828 | 431 |
| 588532 | 2575 | 2594 | Exon 18 | CCCTGTCCAGCAGGAAACCC | 15 | 7810 | 7829 | 432 |
| 588533 | 2576 | 2595 | Exon 18 | CCCCTGTCCAGCAGGAAACC | 29 | 7811 | 7830 | 433 |
| 532810 | 2577 | 2596 | Exon 18 | GCCCCTGTCCAGCAGGAAAC | 74 | 7812 | 7831 | 238 |
| 588534 | 2578 | 2597 | Exon 18 | CGCCCCTGTCCAGCAGGAAA | 21 | 7813 | 7832 | 434 |
| 588535 | 2579 | 2598 | Exon 18 | ACGCCCCTGTCCAGCAGGAA | 16 | 7814 | 7833 | 435 |
| 588536 | 2580 | 2599 | Exon 18 | CACGCCCCTGTCCAGCAGGA | 0 | 7815 | 7834 | 436 |
| 588537 | 2581 | 2600 | Exon 18 | CCACGCCCCTGTCCAGCAGG | 8 | 7816 | 7835 | 437 |
| 588538 | 2582 | 2601 | Exon 18 | CCCACGCCCCTGTCCAGCAG | 10 | 7817 | 7836 | 438 |
| 588539 | 2583 | 2602 | Exon 18 | TCCCACGCCCCTGTCCAGCA | 23 | 7818 | 7837 | 439 |
| 588540 | 2584 | 2603 | Exon 18 | ATCCCACGCCCCTGTCCAGC | 16 | 7819 | 7838 | 440 |
| 588541 | 2585 | 2604 | Exon 18 | AATCCCACGCCCCTGTCCAG | 16 | 7820 | 7839 | 441 |
| 588542 | 2586 | 2605 | Exon 18 | CAATCCCACGCCCCTGTCCA | 12 | 7821 | 7840 | 442 |
| 588543 | 2587 | 2606 | Exon 18 | TCAATCCCACGCCCCTGTCC | 26 | 7822 | 7841 | 443 |
| 588544 | 2588 | 2607 | Exon 18 | TTCAATCCCACGCCCCTGTC | 26 | 7823 | 7842 | 444 |
| 588545 | 2589 | 2608 | Exon 18 | ATTCAATCCCACGCCCCTGT | 31 | 7824 | 7843 | 445 |
| 588546 | 2590 | 2609 | Exon 18 | AATTCAATCCCACGCCCCTG | 22 | 7825 | 7844 | 446 |
| 588547 | 2591 | 2610 | Exon 18 | TAATTCAATCCCACGCCCCT | 12 | 7826 | 7845 | 447 |
| 588548 | 2592 | 2611 | Exon 18 | TTAATTCAATCCCACGCCCC | 20 | 7827 | 7846 | 448 |
| 588549 | 2593 | 2612 | Exon 18 | TTTAATTCAATCCCACGCCC | 26 | 7828 | 7847 | 449 |
| 588550 | 2594 | 2613 | Exon 18 | TTTTAATTCAATCCCACGCC | 32 | 7829 | 7848 | 450 |
| 588551 | 2595 | 2614 | Exon 18 | GTTTTAATTCAATCCCACGC | 48 | 7830 | 7849 | 451 |
| 588552 | 2596 | 2615 | Exon 18 | TGTTTTAATTCAATCCCACG | 57 | 7831 | 7850 | 452 |
| 588553 | 2597 | 2616 | Exon 18 | CTGTTTTAATTCAATCCCAC | 49 | 7832 | 7851 | 453 |
| 588554 | 2598 | 2617 | Exon 18 | GCTGTTTTAATTCAATCCCA | 64 | 7833 | 7852 | 454 |
| 532811 | 2599 | 2618 | Exon 18 | AGCTGTTTTAATTCAATCCC | 78 | 7834 | 7853 | 239 |
| 588555 | 2600 | 2619 | Exon 18 | CAGCTGTTTTAATTCAATCC | 48 | 7835 | 7854 | 455 |
| 588556 | 2601 | 2620 | Exon 18 | GCAGCTGTTTTAATTCAATC | 55 | 7836 | 7855 | 456 |
| 588557 | 2602 | 2621 | Exon 18 | CGCAGCTGTTTTAATTCAAT | 51 | 7837 | 7856 | 457 |
| 588558 | 2603 | 2622 | Exon 18 | TCGCAGCTGTTTTAATTCAA | 51 | 7838 | 7857 | 458 |
| 532917 | 2604 | 2623 | Exon 18 | GTCGCAGCTGTTTTAATTCA | 82 | 7839 | 7858 | 317 |
| 588559 | 2605 | 2624 | Exon 18 | TGTCGCAGCTGTTTTAATTC | 58 | 7840 | 7859 | 459 |
| 588560 | 2606 | 2625 | Exon 18 | TTGTCGCAGCTGTTTTAATT | 72 | 7841 | 7860 | 460 |
| 588561 | 2607 | 2626 | Exon 18 | GTTGTCGCAGCTGTTTTAAT | 75 | 7842 | 7861 | 461 |
| 532952 | 2608 | 2627 | Exon 18 | TGTTGTCGCAGCTGTTTTAA | 39 | 7843 | 7862 | 395 |
| 588562 | 2609 | 2628 | Exon 18/ | TTGTTGTCGCAGCTGTTTTA | 53 | n/a | n/a | 462 |
| | | | Repeat | | | | | |
| 588563 | 2610 | 2629 | Exon 18/ | TTTGTTGTCGCAGCTGTTTT | 62 | n/a | n/a | 463 |
| | | | Repeat | | | | | |
| 588564 | 2611 | 2630 | Exon 18/ | TTTTGTTGTCGCAGCTGTTT | 63 | n/a | n/a | 464 |
| | | | Repeat | | | | | |
| 588565 | 2612 | 2631 | Exon 18/ | TTTTTGTTGTCGCAGCTGTT | 64 | n/a | n/a | 465 |
| | | | Repeat | | | | | |

### Example 122: Dose-Dependent Antisense Inhibition of Human CFB in HepG2 Cells by 5-10-5 MOE Gapmers

Gapmers from studies described above exhibiting in vitro inhibition of CFB mRNA were selected and tested at various doses in HepG2 cells. Cells were plated at a density of 20,000 cells per well and transfected using electroporation with 0.313 μM, 0.625 μM, 1.25 μM, 2.50 μM, 5.00 μM, or 10.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 148

| ISIS | 0.313 | 0.625 | 1.25 | 2.50 | 5.00 | 10.00 | IC₅₀ |
|---|---|---|---|---|---|---|---|
| No | μM | μM | μM | μM | μM | μM | (μM) |
| 532614 | 7 | 13 | 43 | 72 | 65 | 71 | 2.2 |
| 532635 | 12 | 0 | 3 | 28 | 0 | 0 | >10 |
| 532692 | 26 | 0 | 12 | 52 | 55 | 74 | 3.7 |
| 532770 | 21 | 18 | 32 | 73 | 64 | 88 | 1.8 |
| 532775 | 8 | 0 | 26 | 35 | 47 | 59 | 6.2 |
| 532800 | 0 | 5 | 30 | 65 | 50 | 75 | 3.1 |
| 532809 | 12 | 30 | 28 | 40 | 46 | 66 | 4.6 |
| 532810 | 28 | 44 | 32 | 69 | 84 | 95 | 1.2 |
| 532811 | 66 | 83 | 90 | 94 | 97 | 99 | <0.3 |
| 532917 | 64 | 85 | 88 | 96 | 97 | 99 | <0.3 |
| 532952 | 50 | 53 | 68 | 80 | 91 | 94 | 0.4 |

### Example 123: Dose-Dependent Antisense Inhibition of Human CFB in HepG2 Cells

Gapmers from studies described above exhibiting in vitro inhibition of CFB mRNA were selected and tested at various doses in HepG2 cells. The antisense oligonucleotides were tested in a number of experiments with similar culture conditions. The results for each experiment are presented in separate tables shown below. Cells were plated at a density of 20,000 cells per well and transfected using electroporation with 0.08 μM, 0.25 μM, 0.74 μM, 2.22 μM, 6.67 μM, and 20.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 149

| ISIS | 0.08 | 0.25 | 0.74 | 2.22 | 6.67 | 20.00 | IC₅₀ |
|---|---|---|---|---|---|---|---|
| No | μM | μM | μM | μM | μM | μM | (μM) |
| 532811 | 19 | 53 | 81 | 87 | 96 | 97 | 0.2 |
| 588834 | 7 | 42 | 64 | 92 | 98 | 98 | 0.5 |
| 588835 | 11 | 30 | 66 | 89 | 97 | 97 | 0.5 |
| 588836 | 14 | 40 | 61 | 91 | 97 | 97 | 0.5 |
| 588837 | 6 | 39 | 67 | 89 | 96 | 97 | 0.5 |
| 588838 | 0 | 27 | 41 | 81 | 87 | 97 | 1.0 |
| 588842 | 17 | 51 | 68 | 86 | 93 | 95 | 0.3 |
| 588843 | 21 | 38 | 72 | 90 | 95 | 96 | 0.4 |
| 588870 | 9 | 31 | 56 | 88 | 95 | 97 | 0.6 |
| 588871 | 14 | 25 | 47 | 79 | 93 | 97 | 0.7 |
| 588872 | 18 | 28 | 59 | 84 | 92 | 97 | 0.6 |

TABLE 150

| ISIS | 0.08 | 0.25 | 0.74 | 2.22 | 6.67 | 20.00 | IC₅₀ |
|---|---|---|---|---|---|---|---|
| No | μM | μM | μM | μM | μM | μM | (μM) |
| 532811 | 31 | 70 | 89 | 94 | 97 | 97 | 0.1 |
| 588844 | 31 | 60 | 77 | 91 | 95 | 96 | 0.1 |
| 588846 | 32 | 52 | 78 | 89 | 95 | 97 | 0.2 |
| 588847 | 22 | 52 | 77 | 91 | 95 | 97 | 0.2 |
| 588848 | 20 | 40 | 73 | 91 | 96 | 98 | 0.3 |
| 588851 | 40 | 52 | 82 | 94 | 97 | 97 | 0.1 |
| 588854 | 17 | 55 | 59 | 84 | 94 | 96 | 0.4 |
| 588855 | 10 | 32 | 56 | 82 | 93 | 96 | 0.6 |
| 588856 | 13 | 46 | 75 | 90 | 96 | 97 | 0.3 |
| 588857 | 11 | 52 | 73 | 94 | 96 | 97 | 0.3 |
| 588858 | 19 | 48 | 75 | 94 | 97 | 98 | 0.3 |

TABLE 151

| ISIS | 0.08 | 0.25 | 0.74 | 2.22 | 6.67 | 20.00 | IC₅₀ |
|---|---|---|---|---|---|---|---|
| No | μM | μM | μM | μM | μM | μM | (μM) |
| 532811 | 42 | 66 | 88 | 96 | 97 | 98 | 0.1 |
| 588859 | 18 | 46 | 66 | 90 | 96 | 97 | 0.4 |
| 588860 | 55 | 80 | 94 | 97 | 97 | 97 | <0.1 |
| 588861 | 24 | 61 | 86 | 93 | 96 | 97 | 0.2 |
| 588862 | 25 | 64 | 85 | 94 | 96 | 98 | 0.1 |
| 588863 | 50 | 73 | 89 | 96 | 96 | 98 | <0.1 |
| 588864 | 52 | 80 | 92 | 96 | 98 | 98 | <0.1 |
| 588865 | 46 | 72 | 91 | 96 | 96 | 99 | <0.1 |
| 588866 | 47 | 76 | 88 | 96 | 97 | 98 | <0.1 |
| 588867 | 43 | 69 | 83 | 92 | 96 | 99 | 0.1 |
| 588868 | 43 | 56 | 65 | 84 | 93 | 97 | 0.1 |

TABLE 152

| ISIS | 0.08 | 0.25 | 0.74 | 2.22 | 6.67 | 20.00 | IC₅₀ |
|---|---|---|---|---|---|---|---|
| No | μM | μM | μM | μM | μM | μM | (μM) |
| 532810 | 0 | 14 | 38 | 72 | 89 | 96 | 1.2 |
| 532811 | 18 | 54 | 79 | 93 | 96 | 97 | 0.3 |
| 532952 | 19 | 34 | 73 | 87 | 94 | 96 | 0.4 |
| 588534 | 17 | 13 | 44 | 77 | 93 | 97 | 0.9 |
| 588544 | 12 | 43 | 69 | 86 | 89 | 93 | 0.4 |
| 588545 | 17 | 55 | 67 | 86 | 91 | 93 | 0.3 |
| 588546 | 10 | 32 | 67 | 85 | 91 | 93 | 0.6 |
| 588552 | 27 | 54 | 76 | 90 | 94 | 97 | 0.2 |
| 588553 | 32 | 68 | 87 | 93 | 95 | 97 | 0.1 |
| 588560 | 16 | 54 | 76 | 90 | 94 | 96 | 0.3 |
| 588561 | 18 | 45 | 68 | 85 | 93 | 96 | 0.4 |

TABLE 153

| ISIS | 0.08 | 0.25 | 0.74 | 2.22 | 6.67 | 20.00 | IC₅₀ |
|---|---|---|---|---|---|---|---|
| No | μM | μM | μM | μM | μM | μM | (μM) |
| 532811 | 22 | 60 | 82 | 94 | 97 | 98 | 0.2 |
| 588536 | 2 | 38 | 65 | 90 | 96 | 97 | 0.6 |
| 588537 | 12 | 38 | 63 | 87 | 94 | 97 | 0.5 |
| 588547 | 19 | 35 | 61 | 86 | 93 | 97 | 0.5 |
| 588548 | 19 | 36 | 75 | 88 | 95 | 96 | 0.4 |
| 588554 | 0 | 76 | 92 | 95 | 97 | 97 | <0.1 |
| 588555 | 31 | 61 | 89 | 96 | 97 | 98 | 0.1 |
| 588556 | 33 | 56 | 82 | 95 | 94 | 97 | 0.1 |
| 588562 | 12 | 39 | 71 | 87 | 94 | 97 | 0.4 |
| 588563 | 25 | 48 | 72 | 86 | 94 | 96 | 0.3 |
| 588564 | 15 | 33 | 63 | 89 | 91 | 97 | 0.5 |

TABLE 154

| ISIS | 0.08 | 0.25 | 0.74 | 2.22 | 6.67 | 20.00 | IC₅₀ |
|---|---|---|---|---|---|---|---|
| No | μM | μM | μM | μM | μM | μM | (μM) |
| 532811 | 39 | 68 | 86 | 96 | 98 | 98 | 0.1 |
| 588538 | 0 | 40 | 82 | 94 | 97 | 98 | 0.3 |
| 588539 | 34 | 65 | 88 | 95 | 98 | 98 | 0.1 |
| 588540 | 30 | 51 | 81 | 91 | 97 | 98 | 0.2 |
| 588549 | 31 | 57 | 82 | 95 | 96 | 98 | 0.1 |
| 588550 | 34 | 65 | 88 | 96 | 98 | 98 | 0.1 |
| 588551 | 47 | 66 | 87 | 96 | 98 | 99 | <0.1 |
| 588557 | 40 | 84 | 95 | 98 | 98 | 98 | <0.1 |
| 588558 | 45 | 73 | 93 | 97 | 98 | 99 | <0.1 |
| 588559 | 51 | 69 | 83 | 96 | 98 | 99 | <0.1 |
| 588565 | 19 | 56 | 81 | 92 | 96 | 98 | 0.2 |

### Example 124: Dose-Dependent Antisense Inhibition of Human CFB in HepG2 Cells

Gapmers from studies described above exhibiting in vitro inhibition of CFB mRNA were selected and tested at various doses in HepG2 cells. The antisense oligonucleotides were tested in a number of experiments with similar culture conditions. The results for each experiment are presented in separate tables shown below. Cells were plated at a density of 20,000 cells per well and transfected using electroporation with 0.06 μM, 0.25 μM, 1.00 μM, and 4.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN©. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 155

| ISIS | 0.06 | 0.25 | 1.00 | 4.00 | IC₅₀ |
|---|---|---|---|---|---|
| No | μM | μM | μM | μM | (μM) |
| 532917 | 31 | 58 | 87 | 92 | 0.2 |
| 588860 | 18 | 50 | 79 | 93 | 0.3 |
| 599001 | 16 | 28 | 69 | 90 | 0.5 |
| 599024 | 14 | 32 | 74 | 90 | 0.4 |
| 599025 | 0 | 31 | 56 | 92 | 0.7 |
| 599032 | 28 | 44 | 62 | 88 | 0.3 |
| 599033 | 28 | 46 | 80 | 92 | 0.2 |
| 599077 | 8 | 20 | 59 | 80 | 0.8 |
| 599080 | 9 | 33 | 48 | 76 | 0.9 |
| 599086 | 7 | 22 | 53 | 83 | 0.8 |
| 599087 | 21 | 31 | 74 | 87 | 0.4 |
| 599088 | 13 | 37 | 69 | 82 | 0.5 |
| 599089 | 3 | 36 | 55 | 79 | 0.7 |
| 599093 | 25 | 59 | 79 | 88 | 0.2 |
| 599094 | 19 | 29 | 75 | 89 | 0.4 |
| 599095 | 29 | 43 | 67 | 87 | 0.3 |
| 599096 | 23 | 51 | 70 | 88 | 0.3 |
| 599149 | 20 | 53 | 82 | 92 | 0.3 |
| 599188 | 0 | 21 | 62 | 85 | 0.8 |

TABLE 156

| ISIS | 0.06 | 0.25 | 1.00 | 4.00 | IC₅₀ |
|---|---|---|---|---|---|
| No | μM | μM | μM | μM | (μM) |
| 532917 | 0 | 42 | 81 | 91 | 0.4 |
| 588860 | 17 | 49 | 74 | 92 | 0.3 |
| 599155 | 29 | 52 | 67 | 87 | 0.3 |
| 599198 | 3 | 25 | 64 | 89 | 0.6 |
| 599201 | 13 | 26 | 67 | 91 | 0.5 |
| 599202 | 0 | 44 | 72 | 87 | 0.5 |
| 599203 | 22 | 41 | 75 | 88 | 0.3 |
| 599314 | 12 | 34 | 71 | 84 | 0.5 |
| 599316 | 7 | 37 | 66 | 88 | 0.5 |
| 599317 | 8 | 1 | 54 | 83 | 1.0 |
| 599321 | 8 | 33 | 70 | 85 | 0.5 |
| 599322 | 24 | 38 | 66 | 87 | 0.4 |
| 599327 | 22 | 32 | 66 | 89 | 0.4 |
| 599328 | 0 | 31 | 59 | 88 | 0.7 |
| 599330 | 5 | 43 | 67 | 84 | 0.5 |
| 599374 | 23 | 42 | 80 | 91 | 0.3 |
| 599378 | 21 | 57 | 80 | 93 | 0.2 |
| 599380 | 23 | 56 | 82 | 93 | 0.2 |
| 599432 | 17 | 37 | 73 | 93 | 0.4 |

TABLE 157

| ISIS | 0.06 | 0.25 | 1.00 | 4.00 | IC₅₀ |
|---|---|---|---|---|---|
| No | μM | μM | μM | μM | (μM) |
| 532917 | 23 | 65 | 76 | 93 | 0.2 |
| 588860 | 17 | 60 | 76 | 90 | 0.3 |
| 601282 | 48 | 68 | 81 | 88 | 0.1 |
| 601269 | 18 | 59 | 80 | 94 | 0.2 |
| 601276 | 34 | 64 | 81 | 91 | 0.1 |
| 601275 | 14 | 39 | 78 | 90 | 0.4 |
| 601344 | 52 | 84 | 92 | 94 | <0.06 |
| 601383 | 53 | 81 | 86 | 94 | <0.06 |
| 601382 | 41 | 76 | 88 | 94 | 0.1 |
| 601385 | 52 | 74 | 89 | 91 | <0.06 |
| 601332 | 41 | 69 | 86 | 94 | 0.1 |
| 601345 | 36 | 75 | 86 | 95 | 0.1 |
| 601371 | 34 | 72 | 91 | 93 | 0.1 |
| 601384 | 50 | 78 | 91 | 95 | <0.06 |
| 601380 | 28 | 57 | 83 | 92 | 0.2 |
| 601387 | 48 | 61 | 82 | 88 | 0.1 |
| 601341 | 28 | 65 | 83 | 91 | 0.2 |
| 601346 | 31 | 69 | 82 | 93 | 0.1 |
| 601335 | 24 | 56 | 85 | 92 | 0.2 |

TABLE 158

| ISIS | 0.06 | 0.25 | 1.00 | 4.00 | IC₅₀ |
|---|---|---|---|---|---|
| No | μM | μM | μM | μM | (μM) |
| 532917 | 31 | 66 | 86 | 93 | 0.1 |
| 588860 | 28 | 62 | 85 | 94 | 0.2 |
| 599208 | 24 | 50 | 71 | 89 | 0.3 |
| 599261 | 31 | 49 | 81 | 94 | 0.2 |
| 599267 | 41 | 48 | 80 | 88 | 0.2 |
| 599268 | 28 | 56 | 75 | 92 | 0.2 |
| 599313 | 14 | 24 | 71 | 92 | 0.5 |
| 599441 | 24 | 57 | 80 | 87 | 0.2 |
| 599494 | 13 | 55 | 86 | 94 | 0.3 |
| 599552 | 30 | 69 | 93 | 95 | 0.1 |
| 599553 | 34 | 71 | 93 | 96 | 0.1 |
| 599554 | 30 | 74 | 93 | 96 | 0.1 |
| 599568 | 40 | 77 | 90 | 97 | 0.1 |
| 599570 | 61 | 82 | 93 | 96 | <0.06 |
| 599577 | 18 | 62 | 81 | 93 | 0.2 |
| 599581 | 27 | 60 | 80 | 94 | 0.2 |
| 599591 | 49 | 74 | 93 | 96 | <0.06 |
| 599592 | 46 | 76 | 90 | 94 | 0.1 |
| 599593 | 44 | 72 | 91 | 95 | 0.1 |

TABLE 159

| ISIS | 0.06 | 0.25 | 1.00 | 4.00 | IC₅₀ |
|---|---|---|---|---|---|
| No | μM | μM | μM | μM | (μM) |
| 532917 | 25 | 56 | 84 | 92 | 0.2 |
| 588860 | 11 | 51 | 80 | 92 | 0.3 |
| 599547 | 23 | 60 | 82 | 90 | 0.2 |
| 599569 | 42 | 73 | 85 | 88 | 0.1 |
| 599578 | 29 | 49 | 82 | 89 | 0.2 |
| 599582 | 21 | 56 | 78 | 91 | 0.2 |
| 599590 | 24 | 62 | 80 | 90 | 0.2 |
| 601209 | 21 | 49 | 85 | 88 | 0.3 |
| 601210 | 34 | 64 | 86 | 92 | 0.1 |
| 601212 | 46 | 68 | 88 | 90 | 0.1 |
| 601213 | 54 | 80 | 90 | 92 | <0.06 |
| 601214 | 38 | 77 | 88 | 95 | 0.1 |
| 601215 | 42 | 64 | 85 | 92 | 0.1 |
| 601216 | 45 | 57 | 76 | 89 | 0.1 |
| 601264 | 29 | 58 | 86 | 95 | 0.2 |
| 601278 | 51 | 82 | 83 | 93 | <0.06 |
| 601279 | 44 | 80 | 92 | 96 | 0.1 |
| 601280 | 44 | 73 | 87 | 94 | 0.1 |
| 601281 | 51 | 80 | 91 | 94 | <0.06 |

### Example 125: Dose-Dependent Antisense Inhibition of Human CFB in HepG2 Cells

Gapmers from studies described above exhibiting in vitro inhibition of CFB mRNA were selected and tested at various doses in HepG2 cells. Additionally, a deoxy, MOE and (S)-cEt oligonucleotide, ISIS 594430, was designed with the same sequence (CTCCTTCCGAGTCAGC, SEQ ID NO: 549) and target region (target start site 2195 of SEQ ID NO: 1 and target start site 6983 of SED ID NO: 2) as ISIS 588870, another deoxy, MOE, and (S)-cEt oligonucleotide. ISIS 594430 is a 3-10-3 (S)-cEt gapmer.

Cells were plated at a density of 20,000 cells per well and transfected using electroporation with 0.01 μM, 0.04 μM, 0.12 μM, 0.37 μM, 1.11 μM, 3.33 μM, and 10.00 μM concentrations of antisense oligonucleotide, as specified in the Table below. After a treatment period of approximately 16 hours, RNA was isolated from the cells and CFB mRNA levels were measured by quantitative real-time PCR. Human primer probe set RTS3459 was used to measure mRNA levels. CFB mRNA levels were adjusted according to total RNA content, as measured by RIBOGREEN®. Results are presented as percent inhibition of CFB, relative to untreated control cells.

The half maximal inhibitory concentration (IC₅₀) of each oligonucleotide is also presented. CFB mRNA levels were reduced in a dose-dependent manner in antisense oligonucleotide treated cells.

TABLE 160

| ISIS | 0.01 | 0.04 | 0.12 | 0.37 | 1.11 | 3.33 | 10.00 | IC₅₀ |
|---|---|---|---|---|---|---|---|---|
| No | μM | μM | μM | μM | μM | μM | μM | (μM) |
| 588536 | 0 | 0 | 0 | 5 | 45 | 73 | 94 | 1.4 |
| 588548 | 0 | 0 | 0 | 19 | 52 | 78 | 90 | 1.2 |
| 588553 | 0 | 0 | 9 | 42 | 76 | 85 | 94 | 0.6 |
| 588555 | 0 | 52 | 23 | 58 | 78 | 83 | 95 | 0.3 |
| 588847 | 4 | 1 | 18 | 45 | 67 | 84 | 96 | 0.5 |
| 588848 | 0 | 3 | 13 | 38 | 67 | 83 | 95 | 0.6 |
| 594430 | 0 | 0 | 10 | 34 | 50 | 55 | 84 | 1.4 |

### Example 126: Tolerability of MOE Gapmers Targeting Human CFB in CD1 Mice

CD1® mice (Charles River, MA) are a multipurpose mice model, frequently utilized for safety and efficacy testing. The mice were treated with ISIS antisense oligonucleotides selected from studies described above and evaluated for changes in the levels of various plasma chemistry markers.

Study 1 (with 5-10-5 MOE Gapmers)

Groups of seven-week old male CD1 mice were injected subcutaneously once a week for 6 weeks with 100 mg/kg of ISIS oligonucleotide. A group of male CD1 mice was injected subcutaneously once a week for 6 weeks with PBS. One group of mice was injected with subcutaneously once a week for 6 weeks with 100 mg/kg of control oligonucleotide ISIS 141923 (CCTTCCCTGAAGGTTCCTCC, designated herein as SEQ ID NO: 809, 5-10-5 MOE gapmer with no known murine target). Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 161
Plasma chemistry markers in CD1 mice plasma on day 40

| | ALT (IU/L) | AST (IU/L) | BUN (mg/dL) |
|---|---|---|---|
| PBS | 25 | 46 | 20 |
| ISIS 532614 | 513 | 407 | 22 |
| ISIS 532692 | 131 | 130 | 24 |
| ISIS 532770 | 36 | 53 | 25 |
| ISIS 532775 | 193 | 158 | 23 |
| ISIS 532800 | 127 | 110 | 25 |
| ISIS 532809 | 36 | 42 | 22 |
| ISIS 532810 | 229 | 286 | 26 |
| ISIS 532811 | 197 | 183 | 21 |
| ISIS 532917 | 207 | 204 | 27 |
| ISIS 532952 | 246 | 207 | 25 |
| ISIS 141923 | 39 | 67 | 23 |

### Weights

Body weights of the mice were measured on day 40 before sacrificing the mice. Weights of organs, liver, kidney, and spleen were also measured after the mice were sacrificed. The results are presented in the Table below. ISIS oligonucleotides that caused changes in the weights outside the expected range for antisense oligonucleotides were excluded in further studies.

TABLE 162
Weights (g) of CD1 mice on day 40

| | Body | Kidney | Liver | Spleen |
|---|---|---|---|---|
| PBS | 44 | 0.8 | 2.0 | 0.1 |
| ISIS 532614 | 43 | 0.7 | 4.3 | 0.2 |
| ISIS 532692 | 42 | 0.7 | 2.6 | 0.2 |
| ISIS 532770 | 42 | 0.6 | 2.3 | 0.2 |
| ISIS 532775 | 42 | 0.7 | 2.5 | 0.2 |
| ISIS 532800 | 43 | 0.6 | 2.8 | 0.3 |
| ISIS 532809 | 42 | 0.6 | 2.2 | 0.1 |
| ISIS 532810 | 43 | 0.6 | 2.3 | 0.2 |
| ISIS 532811 | 41 | 0.7 | 2.4 | 0.2 |
| ISIS 532917 | 42 | 0.7 | 3.0 | 0.2 |
| ISIS 532952 | 44 | 0.8 | 2.5 | 0.3 |
| ISIS 141923 | 41 | 0.6 | 2.0 | 0.1 |

Study 2 (with 5-10-5 MOE Gapmers)

Groups of six- to eight-week old male CD1 mice were injected subcutaneously once a week for 6 weeks with 100 mg/kg of ISIS oligonucleotide. Two groups of male CD1 mice were injected subcutaneously once a week for 6 weeks with PBS. One group of mice was injected with subcutaneously once a week for 6 weeks with 100 mg/kg of control oligonucleotide ISIS 141923. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 163
Plasma chemistry markers in CD1 mice plasma on day 45

| | ALT | AST | Albumin | BUN |
|---|---|---|---|---|
| | (IU/L) | (IU/L) | (g/dL) | (mg/dL) |
| PBS | 39 | 53 | 2.9 | 29 |
| PBS | 50 | 97 | 2.9 | 30 |
| ISIS 141923 | 163 | 174 | 4.1 | 25 |
| ISIS 532810 | 321 | 297 | 2.5 | 26 |
| ISIS 532952 | 182 | 199 | 2.7 | 27 |
| ISIS 588534 | 276 | 248 | 2.6 | 29 |
| ISIS 588536 | 48 | 60 | 2.9 | 31 |
| ISIS 588537 | 72 | 79 | 4.0 | 25 |
| ISIS 588538 | 63 | 67 | 4.5 | 29 |
| ISIS 588539 | 238 | 177 | 3.9 | 28 |
| ISIS 588545 | 496 | 256 | 4.4 | 24 |
| ISIS 588547 | 323 | 210 | 4.4 | 25 |
| ISIS 588548 | 61 | 63 | 4.2 | 27 |
| ISIS 588549 | 127 | 132 | 4.1 | 23 |
| ISIS 588551 | 302 | 282 | 4.2 | 22 |
| ISIS 588552 | 76 | 98 | 4.0 | 30 |
| ISIS 588558 | 1066 | 521 | 3.9 | 27 |
| ISIS 588559 | 76 | 94 | 4.1 | 26 |
| ISIS 588561 | 502 | 500 | 4.4 | 26 |
| ISIS 588563 | 50 | 99 | 4.4 | 28 |

### Weights

Body weights of the mice were measured on day 42. Weights of organs, liver, kidney, and spleen were also measured after the mice were sacrificed on day 45. The results are presented in the Table below. ISIS oligonucleotides that caused changes in the weights outside the expected range for antisense oligonucleotides were excluded in further studies.

TABLE 164
Weights (g) of CD1 mice on day 40

| | Body | Kidney | Liver | Spleen |
|---|---|---|---|---|
| PBS | 44 | 0.7 | 2.4 | 0.1 |
| PBS | 43 | 0.7 | 2.4 | 0.2 |
| ISIS 141923 | 43 | 0.6 | 2.4 | 0.2 |
| ISIS 532810 | 41 | 0.6 | 1.9 | 0.1 |
| ISIS 532952 | 43 | 0.6 | 2.4 | 0.2 |
| ISIS 588534 | 44 | 0.7 | 2.8 | 0.2 |
| ISIS 588536 | 43 | 0.7 | 2.7 | 0.2 |
| ISIS 588537 | 43 | 0.7 | 2.4 | 0.2 |
| ISIS 588538 | 44 | 0.7 | 2.8 | 0.2 |
| ISIS 588539 | 44 | 0.6 | 2.7 | 0.2 |
| ISIS 588545 | 44 | 0.8 | 3.3 | 0.3 |
| ISIS 588547 | 42 | 0.6 | 3.3 | 0.3 |
| ISIS 588548 | 43 | 0.6 | 2.8 | 0.2 |
| ISIS 588549 | 42 | 0.6 | 2.8 | 0.3 |
| ISIS 588551 | 39 | 0.6 | 2.2 | 0.2 |
| ISIS 588552 | 41 | 0.6 | 2.2 | 0.2 |
| ISIS 588558 | 44 | 0.7 | 3.3 | 0.3 |
| ISIS 588559 | 43 | 0.6 | 2.7 | 0.3 |
| ISIS 588561 | 40 | 0.7 | 2.4 | 0.3 |
| ISIS 588563 | 41 | 0.7 | 2.4 | 0.2 |

Study 3 (with 5-10-5 MOE Gapmers)

Groups of six- to eight-week old male CD1 mice were injected subcutaneously once a week for 6 weeks with 100 mg/kg of ISIS oligonucleotide. Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 165
Plasma chemistry markers in CD1 mice plasma on day 42

| | ALT | AST | Albumin | BUN |
|---|---|---|---|---|
| | (IU/L) | (IU/L) | (g/dL) | (mg/dL) |
| PBS | 37 | 108 | 3.1 | 30 |
| PBS | 45 | 51 | 3.0 | 27 |
| ISIS 588544 | 209 | 168 | 2.9 | 26 |
| ISIS 588546 | 526 | 279 | 3.0 | 22 |
| ISIS 588550 | 82 | 136 | 2.7 | 25 |
| ISIS 588553 | 79 | 105 | 3.0 | 24 |
| ISIS 588554 | 112 | 220 | 3.2 | 19 |
| ISIS 588555 | 95 | 162 | 2.8 | 25 |
| ISIS 588556 | 345 | 236 | 3.0 | 26 |
| ISIS 588557 | 393 | 420 | 2.8 | 24 |
| ISIS 588560 | 109 | 148 | 2.7 | 27 |
| ISIS 588562 | 279 | 284 | 2.8 | 22 |
| ISIS 588564 | 152 | 188 | 3.0 | 23 |
| ISIS 588565 | 247 | 271 | 2.8 | 28 |

### Weights

Body weights of the mice were measured on day 42. Weights of organs, liver, kidney, and spleen were also measured after the mice were sacrificed on day 42. The results are presented in the Table below. ISIS oligonucleotides that caused changes in the weights outside the expected range for antisense oligonucleotides were excluded in further studies.

TABLE 166
Weights (g) of CD1 mice on day 40

| | Body | Kidney | Liver | Spleen |
|---|---|---|---|---|
| PBS | 42 | 0.7 | 2.4 | 0.1 |
| PBS | 41 | 0.7 | 2.4 | 0.2 |
| ISIS 588544 | 44 | 0.6 | 1.9 | 0.1 |
| ISIS 588546 | 43 | 0.6 | 2.4 | 0.2 |
| ISIS 588550 | 41 | 0.7 | 2.8 | 0.2 |
| ISIS 588553 | 44 | 0.7 | 2.7 | 0.2 |
| ISIS 588554 | 40 | 0.7 | 2.4 | 0.2 |
| ISIS 588555 | 44 | 0.7 | 2.8 | 0.2 |
| ISIS 588556 | 39 | 0.6 | 2.7 | 0.2 |
| ISIS 588557 | 41 | 0.8 | 3.3 | 0.3 |
| ISIS 588560 | 38 | 0.6 | 3.2 | 0.3 |
| ISIS 588562 | 41 | 0.6 | 2.8 | 0.2 |
| ISIS 588564 | 40 | 0.6 | 2.8 | 0.3 |
| ISIS 588565 | 39 | 0.6 | 2.2 | 0.2 |

Study 4 (with (S) cEt Gapmers and Deoxy, MOE and (S)-cEt Oligonucleotides)

Groups of ten-week old male CD1 mice were injected subcutaneously once a week for 6 weeks with 50 mg/kg of ISIS oligonucleotide from the studies described above. In addition, two oligonucleotides, ISIS 594431 and ISIS 594432, were designed as 3-10-3 (S)-cEt gapmers and were also tested in this study. ISIS 594431(ACCTCCTTCCGAGTCA, SEQ ID NO: 550) targets the same region as ISIS 588871, a deoxy, MOE and (S)-cEt gapmer (target start site 2197 of SEQ ID NO: 1 and target start site 6985 of SEQ ID NO: 2). ISIS 594432 (TGGTCACATTCCCTTC, SEQ ID NO: 542) targets the same region as ISIS 588872 a deoxy, MOE and (S)-cEt gapmer (target start site 154 of SEQ ID NO: 1 and target start site 1875 of SEQ ID NO: 2).

Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 167
Plasma chemistry markers in CD1 mice plasma on day 42

| | | ALT | AST | Albumin | Creatinine | BUN |
|---|---|---|---|---|---|---|
| | Chemistry | (IU/L) | (IU/L) | (g/dL) | (mg/dL) | (mg/dL) |
| PBS | — | 71 | 77 | 2.7 | 0.2 | 29 |
| PBS | — | 30 | 36 | 2.7 | 0.2 | 26 |
| ISIS 588834 | Deoxy, MOE and (S)-cEt | 436 | 510 | 2.8 | 0.2 | 25 |
| ISIS 588835 | Deoxy, MOE and (S)-cEt | 70 | 98 | 3.0 | 0.2 | 27 |
| ISIS 588836 | Deoxy, MOE and (S)-cEt | 442 | 312 | 2.7 | 0.2 | 27 |
| ISIS 588846 | Deoxy, MOE and (S)-cEt | 50 | 75 | 2.5 | 0.1 | 28 |
| ISIS 588847 | Deoxy, MOE and (S)-cEt | 44 | 71 | 2.6 | 0.1 | 24 |
| ISIS 588848 | Deoxy, MOE and (S)-cEt | 47 | 70 | 2.4 | 0.1 | 27 |
| ISIS 588857 | Deoxy, MOE and (S)-cEt | 1287 | 655 | 2.7 | 0.2 | 26 |
| ISIS 588858 | Deoxy, MOE and (S)-cEt | 1169 | 676 | 2.5 | 0.2 | 26 |
| ISIS 588859 | Deoxy, MOE and (S)-cEt | 1036 | 1300 | 3.2 | 0.2 | 25 |
| ISIS 588861 | Deoxy, MOE and (S)-cEt | 749 | 466 | 3.1 | 0.1 | 24 |
| ISIS 588862 | Deoxy, MOE and (S)-cEt | 1564 | 1283 | 2.9 | 0.2 | 22 |
| ISIS 588863 | Deoxy, MOE and (S)-cEt | 477 | 362 | 2.8 | 0.1 | 23 |
| ISIS 588864 | Deoxy, MOE and (S)-cEt | 118 | 165 | 2.9 | 0.2 | 27 |
| ISIS 588866 | Deoxy, MOE and (S)-cEt | 843 | 784 | 3.2 | 0.2 | 25 |
| ISIS 594430 | 3-10-3 (S)-cEt | 89 | 99 | 2.4 | 0.1 | 28 |
| ISIS 594431 | 3-10-3 (S)-cEt | 590 | 433 | 3.0 | 0.2 | 24 |
| ISIS 594432 | 3-10-3 (S)-cEt | 2595 | 2865 | 2.4 | 0.1 | 25 |

### Weights

Body weights of the mice were measured on day 39. Weights of organs, liver, kidney, and spleen were also measured after the mice were sacrificed on day 42. The results are presented in the Table below. ISIS oligonucleotides that caused changes in the weights outside the expected range for antisense oligonucleotides were excluded in further studies.

TABLE 168
Weights (g) of CD1 mice

| | Chemistry | Body | Kidney | Liver | Spleen |
|---|---|---|---|---|---|
| PBS | — | 37 | 0.6 | 2.1 | 0.1 |
| PBS | — | 45 | 0.7 | 2.5 | 0.2 |
| ISIS 588834 | Deoxy, MOE and (S)-cEt | 40 | 0.6 | 3.2 | 0.2 |
| ISIS 588835 | Deoxy, MOE and (S)-cEt | 38 | 0.7 | 2.8 | 0.3 |
| ISIS 588836 | Deoxy, MOE and (S)-cEt | 41 | 0.7 | 2.3 | 0.2 |
| ISIS 588837 | Deoxy, MOE and (S)-cEt | 38 | 0.6 | 2.4 | 0.3 |
| ISIS 588846 | Deoxy, MOE and (S)-cEt | 39 | 0.6 | 2.3 | 0.2 |
| ISIS 588847 | Deoxy, MOE and (S)-cEt | 40 | 0.7 | 2.5 | 0.2 |
| ISIS 588848 | Deoxy, MOE and (S)-cEt | 43 | 0.7 | 2.6 | 0.3 |
| ISIS 588857 | Deoxy, MOE and (S)-cEt | 39 | 0.6 | 3.3 | 0.2 |
| ISIS 588858 | Deoxy, MOE and (S)-cEt | 37 | 0.6 | 3.4 | 0.2 |
| ISIS 588859 | Deoxy, MOE and (S)-cEt | 41 | 0.7 | 2.5 | 0.3 |
| ISIS 588861 | Deoxy, MOE and (S)-cEt | 39 | 0.6 | 2.6 | 0.4 |
| ISIS 588862 | Deoxy, MOE and (S)-cEt | 34 | 0.6 | 2.5 | 0.4 |
| ISIS 588863 | Deoxy, MOE and (S)-cEt | 40 | 0.6 | 2.7 | 0.3 |
| ISIS 588864 | Deoxy, MOE and (S)-cEt | 40 | 0.7 | 2.3 | 0.2 |
| ISIS 588866 | Deoxy, MOE and (S)-cEt | 45 | 0.7 | 3.0 | 0.2 |
| ISIS 594430 | 3-10-3 (S)-cEt | 39 | 0.6 | 2.2 | 0.2 |
| ISIS 594431 | 3-10-3 (S)-cEt | 36 | 0.6 | 3.2 | 0.2 |
| ISIS 594432 | 3-10-3 (S)-cEt | 31 | 0.4 | 1.9 | 0.1 |

Study 5 (with MOE gapmers, (S) cEt gapmers and deoxy, MOE and (S)-cEt oligonucleotides) Groups of eight- to nine-week old male CD1 mice were injected subcutaneously once a week for 6 weeks with 50 mg/kg of ISIS oligonucleotide. Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 169
Plasma chemistry markers in CD1 mice plasma on day 42

| | | ALT | AST | Albumin | Creatinine | BUN |
|---|---|---|---|---|---|---|
| | Chemistry | (IU/L) | (IU/L) | (g/dL) | (mg/dL) | (mg/dL) |
| PBS | — | 33 | 84 | 2.9 | 0.2 | 28 |
| PBS | — | 32 | 65 | 2.5 | 0.1 | 27 |
| ISIS 532692 | 5-10-5 MOE | 363 | 281 | 3.0 | 0.2 | 30 |
| ISIS 532770 | 5-10-5 MOE | 69 | 100 | 2.9 | 0.1 | 28 |
| ISIS 532775 | 5-10-5 MOE | 371 | 333 | 2.6 | 0.1 | 29 |
| ISIS 532800 | 5-10-5 MOE | 104 | 106 | 2.7 | 0.1 | 31 |
| ISIS 532809 | 5-10-5 MOE | 69 | 127 | 2.8 | 0.1 | 26 |
| ISIS 588540 | 5-10-5 MOE | 66 | 110 | 2.8 | 0.1 | 26 |
| ISIS 588838 | 3-10-3 (S)-cEt | 391 | 330 | 2.9 | 0.1 | 25 |
| ISIS 588842 | Deoxy, MOE and (S)-cEt | 224 | 264 | 2.6 | 0.1 | 26 |
| ISIS 588843 | 3-10-3 (S)-cEt | 185 | 160 | 2.8 | 0.1 | 24 |
| ISIS 588844 | Deoxy, MOE and (S)-cEt | 304 | 204 | 2.7 | 0.1 | 25 |
| ISIS 588851 | Deoxy, MOE and (S)-cEt | 186 | 123 | 2.7 | 0.1 | 31 |
| ISIS 588854 | Deoxy, MOE and (S)-cEt | 1232 | 925 | 2.7 | 0.1 | 25 |
| ISIS 588855 | Deoxy, MOE and (S)-cEt | 425 | 321 | 2.7 | 0.1 | 28 |
| ISIS 588856 | Deoxy, MOE and (S)-cEt | 78 | 101 | 2.4 | 0.1 | 31 |
| ISIS 588865 | Deoxy, MOE and (S)-cEt | 126 | 145 | 2.5 | 0.1 | 23 |
| ISIS 588867 | Deoxy, MOE and (S)-cEt | 108 | 112 | 2.5 | 0.1 | 32 |
| ISIS 588868 | Deoxy, MOE and (S)-cEt | 61 | 124 | 2.5 | 0.1 | 28 |
| ISIS 588870 | Deoxy, MOE and (S)-cEt | 48 | 69 | 2.4 | 0.1 | 31 |
| ISIS 588871 | Deoxy, MOE and (S)-cEt | 723 | 881 | 2.5 | 0.1 | 24 |
| ISIS 588872 | Deoxy, MOE and (S)-cEt | 649 | 654 | 2.7 | 0.1 | 26 |

### Weights

Body weights of the mice were measured on day 40. Weights of organs, liver, kidney, and spleen were also measured after the mice were sacrificed on day 42. The results are presented in the Table below. ISIS oligonucleotides that caused changes in the weights outside the expected range for antisense oligonucleotides were excluded in further studies.

TABLE 170
Weights (g) of CD1 mice

| | Chemistry | Body | Kidney | Liver | Spleen |
|---|---|---|---|---|---|
| PBS | — | 46 | 0.7 | 2.3 | 0.2 |
| PBS | — | 44 | 0.7 | 2.3 | 0.2 |
| ISIS 532692 | 5-10-5 MOE | 44 | 0.6 | 2.8 | 0.2 |
| ISIS 532770 | 5-10-5 MOE | 43 | 0.6 | 2.2 | 0.2 |
| ISIS 532775 | 5-10-5 MOE | 43 | 0.6 | 2.8 | 0.2 |
| ISIS 532800 | 5-10-5 MOE | 47 | 0.7 | 2.9 | 0.2 |
| ISIS 532809 | 5-10-5 MOE | 44 | 0.7 | 2.6 | 0.2 |
| ISIS 588540 | 5-10-5 MOE | 44 | 0.7 | 2.5 | 0.2 |
| ISIS 588838 | 3-10-3 (S)-cEt | 45 | 0.7 | 3.1 | 0.2 |
| ISIS 588842 | Deoxy, MOE and (S)-cEt | 41 | 0.6 | 2.6 | 0.2 |
| ISIS 588843 | 3-10-3 (S)-cEt | 43 | 0.7 | 2.9 | 0.2 |
| ISIS 588844 | Deoxy, MOE and (S)-cEt | 43 | 0.7 | 2.8 | 0.2 |
| ISIS 588851 | Deoxy, MOE and (S)-cEt | 46 | 0.6 | 2.6 | 0.2 |
| ISIS 588854 | Deoxy, MOE and (S)-cEt | 45 | 0.7 | 4.1 | 0.2 |
| ISIS 588855 | Deoxy, MOE and (S)-cEt | 44 | 0.7 | 2.9 | 0.3 |
| ISIS 588856 | Deoxy, MOE and (S)-cEt | 44 | 0.7 | 3.2 | 0.2 |
| ISIS 588865 | Deoxy, MOE and (S)-cEt | 45 | 0.7 | 2.6 | 0.3 |
| ISIS 588867 | Deoxy, MOE and (S)-cEt | 46 | 0.7 | 3.2 | 0.3 |
| ISIS 588868 | Deoxy, MOE and (S)-cEt | 42 | 0.7 | 2.9 | 0.3 |
| ISIS 588870 | Deoxy, MOE and (S)-cEt | 43 | 0.6 | 2.2 | 0.2 |
| ISIS 588871 | Deoxy, MOE and (S)-cEt | 41 | 0.7 | 3.1 | 0.2 |
| ISIS 588872 | Deoxy, MOE and (S)-cEt | 39 | 0.6 | 3.2 | 0.3 |

Study 6 (with Deoxy, MOE and (S)-cEt Oligonucleotides)

Groups of eight- to nine-week old male CD1 mice were injected subcutaneously once a week for 6 weeks with 50 mg/kg of deoxy, MOE, and (S)-cEt oligonucleotides. Two groups of male CD1 mice were injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, bilirubin, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 171
Plasma chemistry markers in CD1 mice plasma on day 45

| | ALT | AST | Albumin | Creatinine | Bilirubin | BUN |
|---|---|---|---|---|---|---|
| | (IU/L) | (IU/L) | (g/dL) | (mg/dL) | (mg/dL) | (mg/dL) |
| PBS | 39 | 78 | 3.4 | 0.2 | 0.2 | 31 |
| PBS | 37 | 59 | 2.9 | 0.1 | 0.2 | 27 |
| ISIS 599552 | 167 | 208 | 3.0 | 0.1 | 0.2 | 32 |
| ISIS 599553 | 43 | 86 | 2.9 | 0.1 | 0.2 | 28 |
| ISIS 599554 | 57 | 101 | 2.2 | 0.2 | 0.2 | 31 |
| ISIS 599569 | 469 | 530 | 3.5 | 0.2 | 0.3 | 27 |
| ISIS 599577 | 37 | 84 | 2.9 | 0.1 | 0.1 | 31 |
| ISIS 599578 | 45 | 104 | 2.8 | 0.1 | 0.2 | 30 |
| ISIS 599581 | 54 | 88 | 3.1 | 0.1 | 0.2 | 31 |
| ISIS 599590 | 1741 | 1466 | 3.1 | 0.1 | 0.3 | 25 |
| ISIS 599591 | 2230 | 1183 | 3.2 | 0.1 | 0.3 | 27 |
| ISIS 601209 | 68 | 104 | 2.9 | 0.1 | 0.2 | 30 |
| ISIS 601212 | 1795 | 968 | 3.2 | 0.1 | 0.3 | 22 |
| ISIS 601215 | 424 | 385 | 3.1 | 0.1 | 0.4 | 25 |
| ISIS 601216 | 90 | 125 | 2.9 | 0.1 | 0.2 | 29 |
| ISIS 601276 | 946 | 366 | 2.9 | 0.1 | 0.5 | 31 |
| ISIS 601282 | 831 | 540 | 3.3 | 0.2 | 0.2 | 32 |

### Weights

Body weights of the mice were measured on day 40. Weights of organs, liver, kidney, and spleen were also measured after the mice were sacrificed on day 45. The results are presented in the Table below. ISIS oligonucleotides that caused changes in the weights outside the expected range for antisense oligonucleotides were excluded in further studies.

TABLE 172
Weights (g) of CD1 mice

| | Body | Kidney | Liver | Spleen |
|---|---|---|---|---|
| PBS | 40 | 0.7 | 2.1 | 0.2 |
| PBS | 42 | 0.8 | 2.3 | 0.2 |
| ISIS 599552 | 38 | 0.6 | 2.3 | 0.2 |
| ISIS 599553 | 39 | 0.7 | 2.2 | 0.2 |
| ISIS 599554 | 39 | 0.7 | 2.4 | 0.2 |
| ISIS 599569 | 39 | 0.7 | 2.2 | 0.2 |
| ISIS 599577 | 41 | 0.7 | 2.5 | 0.2 |
| ISIS 599578 | 37 | 0.6 | 2.0 | 0.2 |
| ISIS 599581 | 40 | 0.6 | 2.5 | 0.2 |
| ISIS 599590 | 34 | 0.6 | 3.5 | 0.2 |
| ISIS 599591 | 38 | 0.8 | 2.7 | 0.2 |
| ISIS 601209 | 42 | 0.7 | 2.6 | 0.3 |
| ISIS 601212 | 38 | 0.6 | 2.9 | 0.2 |
| ISIS 601215 | 36 | 0.7 | 2.6 | 0.2 |
| ISIS 601216 | 42 | 0.6 | 2.7 | 0.2 |
| ISIS 601276 | 42 | 0.7 | 3.2 | 0.2 |
| ISIS 601282 | 38 | 0.7 | 3.2 | 0.2 |

Study 7 (with MOE Gapmers and Deoxy, MOE and (S)-cEt Oligonucleotides)

Groups of eight- to nine-week old male CD1 mice were injected subcutaneously once a week for 6 weeks with 100 mg/kg of ISIS oligonucleotides. One group of male CD1 mice was injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below. ISIS oligonucleotides that caused changes in the levels of any of the liver or kidney function markers outside the expected range for antisense oligonucleotides were excluded in further studies.

TABLE 173
Plasma chemistry markers in CD1 mice plasma on day 45

| | | ALT | AST | Albumin | Creatinine | BUN |
|---|---|---|---|---|---|---|
| | Chemistry | (IU/L) | (IU/L) | (g/dL) | (mg/dL) | (mg/dL) |
| PBS | — | 120 | 102 | 2.7 | 0.2 | 26 |
| ISIS 588842 | Deoxy, MOE and (S)-cEt | 177 | 164 | 2.7 | 0.1 | 23 |
| ISIS 588843 | Deoxy, MOE and (S)-cEt | 98 | 194 | 2.7 | 0.1 | 24 |
| ISIS 588851 | Deoxy, MOE and (S)-cEt | 91 | 142 | 2.6 | 0.1 | 23 |
| ISIS 588856 | Deoxy, MOE and (S)-cEt | 78 | 110 | 2.7 | 0.1 | 23 |
| ISIS 599024 | 3-10-4 MOE | 91 | 108 | 2.7 | 0.1 | 23 |
| ISIS 599087 | 5-7-5 MOE | 198 | 183 | 2.6 | 0.2 | 28 |
| ISIS 599093 | 5-7-5 MOE | 3285 | 2518 | 2.6 | 0.2 | 24 |
| ISIS 599149 | 4-8-5 MOE | 30 | 64 | 2.9 | 0.2 | 25 |
| ISIS 599155 | 4-8-5 MOE | 145 | 189 | 2.6 | 0.2 | 25 |
| ISIS 599202 | 5-8-5 MOE | 150 | 128 | 2.8 | 0.2 | 23 |
| ISIS 599203 | 5-8-5 MOE | 111 | 127 | 2.8 | 0.2 | 24 |
| ISIS 599208 | 5-8-5 MOE | 146 | 178 | 2.9 | 0.2 | 22 |
| ISIS 599261 | 3-10-5 MOE | 144 | 165 | 2.8 | 0.2 | 26 |
| ISIS 599267 | 3-10-5 MOE | 96 | 132 | 2.6 | 0.2 | 27 |
| ISIS 599268 | 3-10-5 MOE | 87 | 115 | 2.6 | 0.1 | 23 |
| ISIS 599322 | 6-7-6 MOE | 115 | 138 | 2.7 | 0.1 | 22 |
| ISIS 599374 | 5-9-5 MOE | 375 | 271 | 2.6 | 0.1 | 21 |
| ISIS 599378 | 5-9-5 MOE | 77 | 99 | 2.7 | 0.1 | 23 |
| ISIS 599441 | 6-8-6 MOE | 150 | 250 | 2.9 | 0.1 | 23 |

### Weights

Body weights of the mice were measured on day 44. Weights of organs, liver, kidney, and spleen were also measured after the mice were sacrificed on day 49. The results are presented in the Table below. ISIS oligonucleotides that caused changes in the weights outside the expected range for antisense oligonucleotides were excluded in further studies.

TABLE 174
Weights (g) of CD1 mice

| | Chemistry | Body | Kidney | Liver | Spleen |
|---|---|---|---|---|---|
| PBS | — | 39 | 0.6 | 1.9 | 0.1 |
| ISIS 588842 | Deoxy, MOE and (S)-cEt | 38 | 0.5 | 2.1 | 0.1 |
| ISIS 588843 | Deoxy, MOE and (S)-cEt | 41 | 0.6 | 2.4 | 0.2 |
| ISIS 588851 | Deoxy, MOE and (S)-cEt | 42 | 0.6 | 2.2 | 0.2 |
| ISIS 588856 | Deoxy, MOE and (S)-cEt | 42 | 0.7 | 2.6 | 0.2 |
| ISIS 599024 | 3-10-4 MOE | 41 | 0.6 | 4.0 | 0.2 |
| ISIS 599087 | 5-7-5 MOE | 44 | 0.8 | 2.6 | 0.3 |
| ISIS 599093 | 5-7-5 MOE | 39 | 0.6 | 2.3 | 0.2 |
| ISIS 599149 | 4-8-5 MOE | 42 | 0.7 | 2.8 | 0.2 |
| ISIS 599155 | 4-8-5 MOE | 41 | 0.7 | 2.1 | 0.2 |
| ISIS 599202 | 5-8-5 MOE | 43 | 0.6 | 2.6 | 0.2 |
| ISIS 599203 | 5-8-5 MOE | 42 | 0.6 | 2.6 | 0.2 |
| ISIS 599208 | 5-8-5 MOE | 40 | 0.6 | 2.1 | 0.2 |
| ISIS 599261 | 3-10-5 MOE | 39 | 0.7 | 3.4 | 0.3 |
| ISIS 599267 | 3-10-5 MOE | 42 | 0.8 | 2.5 | 0.3 |
| ISIS 599268 | 3-10-5 MOE | 41 | 0.7 | 2.1 | 0.2 |
| ISIS 599322 | 6-7-6 MOE | 43 | 0.6 | 2.2 | 0.2 |
| ISIS 599374 | 5-9-5 MOE | 37 | 0.6 | 2.2 | 0.2 |
| ISIS 599378 | 5-9-5 MOE | 43 | 0.7 | 2.7 | 0.2 |
| ISIS 599441 | 6-8-6 MOE | 42 | 0.6 | 2.5 | 0.3 |

Study 8 (with MOE Gapmers, Deoxy, MOE and (S)-cEt Oligonucleotides, and (S)-cEt Gapmers)

Groups of eight- to nine-week old male CD1 mice were injected subcutaneously once a week for 6 weeks with 100 mg/kg of MOE gapmers, or 50 mg/kg of deoxy, MOE and (S)-cEt oligonucleotides or (S)-cEt gapmers. One group of male CD s mice was injected subcutaneously once a week for 6 weeks with PBS. Mice were euthanized 48 hours after the last dose, and organs and plasma were harvested for further analysis.

### Plasma Chemistry Markers

To evaluate the effect of ISIS oligonucleotides on liver and kidney function, plasma levels of transaminases, albumin, creatinine, and BUN were measured using an automated clinical chemistry analyzer (Hitachi Olympus AU400e, Melville, NY). The results are presented in the Table below.

TABLE 175
Plasma chemistry markers in CD1 mice plasma on day 43

| | | Dose | ALT | AST | Albumin | Creatinine | BUN |
|---|---|---|---|---|---|---|---|
| | Chemistry | (mg/kg/wk) | (IU/L) | (IU/L) | (g/dL) | (mg/dL) | (mg/dL) |
| PBS | — | — | 37 | 57 | 2.5 | 0.08 | 26 |
| ISIS 532770 | 5-10-5 MOE | 100 | 57 | 73 | 2.5 | 0.07 | 24 |
| ISIS 532800 | 5-10-5 MOE | 100 | 74 | 126 | 2.8 | 0.10 | 26 |
| ISIS 532809 | 5-10-5 MOE | 100 | 83 | 73 | 2.5 | 0.07 | 23 |
| ISIS 588540 | 5-10-5 MOE | 100 | 106 | 102 | 2.7 | 0.09 | 27 |
| ISIS 588544 | 5-10-5 MOE | 100 | 66 | 62 | 2.6 | 0.10 | 24 |
| ISIS 588548 | 5-10-5 MOE | 100 | 48 | 67 | 2.6 | 0.08 | 23 |
| ISIS 588550 | 5-10-5 MOE | 100 | 65 | 106 | 2.5 | 0.10 | 25 |
| ISIS 588553 | 5-10-5 MOE | 100 | 78 | 90 | 2.6 | 0.09 | 25 |
| ISIS 588555 | 5-10-5 MOE | 100 | 94 | 89 | 2.5 | 0.08 | 23 |
| ISIS 588848 | Deoxy, MOE | 50 | 38 | 54 | 2.3 | 0.07 | 25 |
| | and (S)-cEt | | | | | | |
| ISIS 594430 | 3-10-3 (S)-cEt | 50 | 63 | 72 | 2.5 | 0.10 | 27 |

### Weights

Body weights of the mice were measured on day 36. Weights of organs, liver, kidney, and spleen were also measured after the mice were sacrificed on day 43. The results for the organ weights were expressed as a ratio to the body weights and normalized to the PBS control ratio.

TABLE 176
Organ Weights/Body weight (BW) of CD1 mice

| | | Dose | Kidney/ | Liver/ | Spleen/ |
|---|---|---|---|---|---|
| | Chemistry | (mg/kg/wk) | BW | BW | BW |
| PBS | — | — | 1.0 | 1.0 | 1.0 |
| ISIS 532770 | 5-10-5 MOE | 100 | 1.4 | 1.1 | 1.0 |
| ISIS 532800 | 5-10-5 MOE | 100 | 1.5 | 1.1 | 0.9 |
| ISIS 532809 | 5-10-5 MOE | 100 | 1.3 | 1.2 | 0.9 |
| ISIS 588540 | 5-10-5 MOE | 100 | 1.3 | 1.2 | 1.0 |
| ISIS 588544 | 5-10-5 MOE | 100 | 1.6 | 1.1 | 1.0 |
| ISIS 588548 | 5-10-5 MOE | 100 | 1.7 | 1.2 | 1.0 |
| ISIS 588550 | 5-10-5 MOE | 100 | 1.5 | 1.2 | 1.0 |
| ISIS 588553 | 5-10-5 MOE | 100 | 1.5 | 1.0 | 0.8 |
| ISIS 588555 | 5-10-5 MOE | 100 | 1.8 | 1.2 | 1.0 |
| ISIS 588848 | Deoxy, MOE | 50 | 1.3 | 1.0 | 0.9 |
| | and (S)-cEt | | | | |
| ISIS 594430 | 3-10-3 (S)-cEt | 50 | 1.4 | 1.1 | 0.9 |

### Cytokine Assays

Blood obtained from all mice groups were sent to Antech Diagnostics for measurements of the various cytokine levels, such as IL-6, MDC, MIP1β, IP-10, MCP1, MIP-1α, and RANTES. The results are presented in Table 54.

TABLE 177
Cytokine levels (pg/mL) in CD1 mice plasma

| | Chemistry | IL-6 | MDC | MIP1β | IP-10 | MCP1 | MIP-1α | RANTES |
|---|---|---|---|---|---|---|---|---|
| PBS | — | 70 | 16 | 23 | 20 | 17 | 6 | 2 |
| ISIS 532770 | 5-10-5 MOE | 101 | 18 | 146 | 116 | 101 | 24 | 6 |
| ISIS 532800 | 5-10-5 MOE | 78 | 17 | 83 | 53 | 105 | 1 | 3 |
| ISIS 532809 | 5-10-5 MOE | 66 | 19 | 60 | 32 | 55 | 20 | 4 |
| ISIS 588540 | 5-10-5 MOE | 51 | 18 | 126 | 70 | 75 | 4 | 3 |
| ISIS 588544 | 5-10-5 MOE | 157 | 14 | 94 | 34 | 102 | 1 | 3 |
| ISIS 588548 | 5-10-5 MOE | 164 | 12 | 90 | 66 | 84 | 10 | 4 |
| ISIS 588550 | 5-10-5 MOE | 58 | 21 | 222 | 124 | 157 | 3 | 5 |
| ISIS 588553 | 5-10-5 MOE | 62 | 14 | 183 | 60 | 103 | 9 | 4 |
| ISIS 588555 | 5-10-5 MOE | 70 | 19 | 172 | 171 | 178 | 16 | 9 |
| ISIS 588848 | Deoxy, MOE | 59 | 13 | 61 | 27 | 63 | 12 | 4 |
| | and (S)-cEt | | | | | | | |
| ISIS 594430 | 3-10-3 (S)-cEt | 48 | 14 | 56 | 38 | 85 | 10 | 3 |

### Hematology Assays

Blood obtained from all mice groups were sent to Antech Diagnostics for measurements of hematocrit (HCT), as well as of the various blood cells, such as WBC, RBC, and platelets, and total hemoglobin (Hb) content. The results are presented in Table 55.

TABLE 178
Hematology markers in CD1 mice plasma

| | | HCT | Hb | WBC | RBC | Platelets |
|---|---|---|---|---|---|---|
| | Chemistry | (%) | (g/dL) | (10³/μL) | (10⁶/μL) | (10³/μL) |
| PBS | — | 46 | 15 | 7 | 9 | 960 |
| ISIS 532770 | 5-10-5 MOE | 45 | 14 | 5 | 9 | 879 |
| ISIS 532800 | 5-10-5 MOE | 45 | 14 | 5 | 9 | 690 |
| ISIS 532809 | 5-10-5 MOE | 46 | 14 | 6 | 9 | 1005 |
| ISIS 588540 | 5-10-5 MOE | 49 | 15 | 6 | 10 | 790 |
| ISIS 588544 | 5-10-5 MOE | 36 | 11 | 7 | 7 | 899 |
| ISIS 588548 | 5-10-5 MOE | 46 | 14 | 6 | 9 | 883 |
| ISIS 588550 | 5-10-5 MOE | 42 | 13 | 8 | 8 | 721 |
| ISIS 588553 | 5-10-5 MOE | 45 | 14 | 6 | 9 | 719 |
| ISIS 588555 | 5-10-5 MOE | 43 | 13 | 8 | 9 | 838 |
| ISIS 588848 | Deoxy, MOE | 40 | 15 | 8 | 10 | 840 |
| | and (S)-cEt | | | | | |
| ISIS 594430 | 3-10-3 (S)-cEt | 45 | 14 | 8 | 9 | 993 |

### Example 127: Tolerability of Antisense Oligonucleotides Targeting Human CFB in Sprague-Dawley Rats

Sprague-Dawley rats are a multipurpose model used for safety and efficacy evaluations. The rats were treated with ISIS antisense oligonucleotides from the studies described in the Examples above and evaluated for changes in the levels of various plasma chemistry markers.

Study 1 (with 5-10-5 MOE Gapmers)

Male Sprague-Dawley rats, seven- to eight-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of 5-10-5 MOE gapmers. One control group of 6 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 179
Liver function markers in Sprague-Dawley rats

| | ALT (IU/L) | AST (IU/L) |
|---|---|---|
| PBS | 66 | 134 |
| ISIS 588544 | 101 | 329 |
| ISIS 588550 | 69 | 157 |
| ISIS 588553 | 88 | 304 |
| ISIS 588554 | 202 | 243 |
| ISIS 588555 | 94 | 113 |
| ISIS 588556 | 102 | 117 |
| ISIS 588560 | 206 | 317 |
| ISIS 588564 | 292 | 594 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 180
Kidney function markers (mg/dL) in Sprague-Dawley rats

| | BUN | Creatinine |
|---|---|---|
| PBS | 18 | 3.5 |
| ISIS 588544 | 21 | 3.1 |
| ISIS 588550 | 21 | 3.0 |
| ISIS 588553 | 22 | 2.8 |
| ISIS 588554 | 23 | 3.0 |
| ISIS 588555 | 22 | 3.5 |
| ISIS 588556 | 21 | 3.2 |
| ISIS 588560 | 26 | 2.4 |
| ISIS 588564 | 24 | 2.7 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 181
Weights (g)

| | Body | Liver | Spleen | Kidney |
|---|---|---|---|---|
| PBS | 422 | 16 | 1.2 | 3.9 |
| ISIS 588544 | 353 | 15 | 1.7 | 2.9 |
| ISIS 588550 | 321 | 14 | 2.1 | 3.2 |
| ISIS 588553 | 313 | 15 | 2.3 | 3.2 |
| ISIS 588554 | 265 | 11 | 1.6 | 2.7 |
| ISIS 588555 | 345 | 14 | 1.4 | 3.3 |
| ISIS 588556 | 328 | 13 | 1.7 | 3.1 |
| ISIS 588560 | 270 | 13 | 2.4 | 3.0 |
| ISIS 588564 | 253 | 12 | 2.9 | 3.0 |

Study 2 (with Deoxy, MOE and (S)-cEt Oligonucleotides)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of deoxy, MOE, and (S)-cEt oligonucleotides. Two control groups of 3 rats each were injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase), and albumin were measured and the results are presented in the Table below. ISIS oligonucleotides that caused changes in the levels of any markers of liver function outside the expected range for antisense oligonucleotides were excluded in further studies.

TABLE 182
Liver function markers in Sprague-Dawley rats

| | ALT (IU/L) | AST (IU/L) | Albumin (g/dL) |
|---|---|---|---|
| PBS | 55 | 150 | 3.4 |
| PBS | 64 | 91 | 3.5 |
| ISIS 588554 | 52 | 92 | 3.2 |
| ISIS 588835 | 971 | 844 | 4.1 |
| ISIS 588842 | 317 | 359 | 3.8 |
| ISIS 588843 | 327 | 753 | 2.9 |
| ISIS 588846 | 70 | 111 | 3.2 |
| ISIS 588847 | 65 | 100 | 3.0 |
| ISIS 588864 | 91 | 109 | 3.0 |
| ISIS 594430 | 85 | 106 | 3.7 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 183
Kidney function markers (mg/dL) in Sprague-Dawley rats

| | BUN | Creatinine |
|---|---|---|
| PBS | 17 | 0.4 |
| PBS | 21 | 0.4 |
| ISIS 588554 | 20 | 0.4 |
| ISIS 588835 | 23 | 0.5 |
| ISIS 588842 | 22 | 0.4 |
| ISIS 588843 | 51 | 0.4 |
| ISIS 588846 | 25 | 0.5 |
| ISIS 588847 | 23 | 0.5 |
| ISIS 588864 | 23 | 0.4 |
| ISIS 594430 | 22 | 0.5 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 184
Weights (g)

| | Body | Liver | Spleen | Kidney |
|---|---|---|---|---|
| PBS | 466 | 16 | 0.9 | 3.8 |
| PBS | 485 | 16 | 0.9 | 3.6 |
| ISIS 588554 | 393 | 15 | 2.3 | 2.6 |
| ISIS 588835 | 387 | 16 | 1.0 | 3.3 |
| ISIS 588842 | 414 | 22 | 1.5 | 3.7 |
| ISIS 588843 | 427 | 20 | 2.5 | 4.2 |
| ISIS 588846 | 366 | 16 | 2.1 | 3.3 |
| ISIS 588847 | 402 | 15 | 1.6 | 3.1 |
| ISIS 588864 | 364 | 15 | 2.1 | 3.8 |
| ISIS 594430 | 420 | 16 | 1.2 | 3.6 |

Study 3 (with MOE Gapmers)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of MOE gapmers. One control group of 6 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 43 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 185
Liver function markers in Sprague-Dawley rats

| | | ALT | AST | Albumin |
|---|---|---|---|---|
| | Chemistry | (IU/L) | (IU/L) | (g/dL) |
| PBS | — | 52 | 110 | 3.7 |
| ISIS 588563 | 5-10-5 MOE | 175 | 291 | 2.9 |
| ISIS 599024 | 3-10-4 MOE | 139 | 173 | 1.4 |
| ISIS 599093 | 5-7-5 MOE | 116 | 238 | 2.6 |
| ISIS 599149 | 4-8-5 MOE | 232 | 190 | 3.4 |
| ISIS 599155 | 4-8-5 MOE | 108 | 215 | 2.5 |
| ISIS 599202 | 5-8-5 MOE | 65 | 86 | 3.5 |
| ISIS 599203 | 5-8-5 MOE | 71 | 97 | 3.1 |
| ISIS 599208 | 5-8-5 MOE | 257 | 467 | 1.9 |
| ISIS 599261 | 3-10-5 MOE | 387 | 475 | 1.5 |
| ISIS 599267 | 3-10-5 MOE | 201 | 337 | 2.7 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 186
Kidney function markers (mg/dL) in Sprague-Dawley rats

| | Chemistry | BUN | Creatinine |
|---|---|---|---|
| PBS | — | 16 | 0.3 |
| ISIS 588563 | 5-10-5 MOE | 26 | 0.4 |
| ISIS 599024 | 3-10-4 MOE | 135 | 1.2 |
| ISIS 599093 | 5-7-5 MOE | 29 | 0.4 |
| ISIS 599149 | 4-8-5 MOE | 23 | 0.4 |
| ISIS 599155 | 4-8-5 MOE | 29 | 0.4 |
| ISIS 599202 | 5-8-5 MOE | 19 | 0.4 |
| ISIS 599203 | 5-8-5 MOE | 22 | 0.4 |
| ISIS 599208 | 5-8-5 MOE | 26 | 0.3 |
| ISIS 599261 | 3-10-5 MOE | 228 | 1.6 |
| ISIS 599267 | 3-10-5 MOE | 24 | 0.4 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 187
Weights (g)

| | Chemistry | Body | Liver | Spleen | Kidney |
|---|---|---|---|---|---|
| PBS | — | 471 | 16 | 1.0 | 4.1 |
| ISIS 588563 | 5-10-5 MOE | 311 | 16 | 3.4 | 4.1 |
| ISIS 599024 | 3-10-4 MOE | 297 | 11 | 1.0 | 3.5 |
| ISIS 599093 | 5-7-5 MOE | 332 | 18 | 4.1 | 5.0 |
| ISIS 599149 | 4-8-5 MOE | 388 | 16 | 2.3 | 3.7 |
| ISIS 599155 | 4-8-5 MOE | 290 | 15 | 2.9 | 4.5 |
| ISIS 599202 | 5-8-5 MOE | 359 | 13 | 1.3 | 3.2 |
| ISIS 599203 | 5-8-5 MOE | 334 | 14 | 1.8 | 3.3 |
| ISIS 599208 | 5-8-5 MOE | 353 | 29 | 4.7 | 4.6 |
| ISIS 599261 | 3-10-5 MOE | 277 | 10 | 0.9 | 3.2 |
| ISIS 599267 | 3-10-5 MOE | 344 | 21 | 3.9 | 4.7 |

Study 4 (with MOE Gapmers)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of MOE gapmers. One control group of 6 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 188
Liver function markers in Sprague-Dawley rats

| | | ALT | AST | Albumin |
|---|---|---|---|---|
| | Chemistry | (IU/L) | (IU/L) | (g/dL) |
| PBS | — | 48 | 77 | 3.9 |
| ISIS 532800 | 5-10-5 MOE | 72 | 111 | 3.4 |
| ISIS 532809 | 5-10-5 MOE | 59 | 89 | 3.8 |
| ISIS 588540 | 5-10-5 MOE | 146 | 259 | 3.8 |
| ISIS 599268 | 3-10-5 MOE | 175 | 206 | 2.7 |
| ISIS 599322 | 6-7-6 MOE | 523 | 567 | 3.3 |
| ISIS 599374 | 5-9-5 MOE | 114 | 176 | 3.0 |
| ISIS 599378 | 5-9-5 MOE | 124 | 116 | 3.2 |
| ISIS 599380 | 5-9-5 MOE | 148 | 210 | 3.4 |
| ISIS 599441 | 6-8-6 MOE | 51 | 91 | 2.6 |

### Kidney Function

To evaluate the effect of ISIS oligonucleotides on kidney function, plasma levels of blood urea nitrogen (BUN) and creatinine were measured using an automated clinical chemistry analyzer (Hitachi Olympus AU400e, Melville, NY). Results are presented in the Table below, expressed in mg/dL. ISIS oligonucleotides that caused changes in the levels of any of the kidney function markers outside the expected range for antisense oligonucleotides were excluded in further studies.

TABLE 189
Kidney function markers (mg/dL) in Sprague-Dawley rats

| | Chemistry | BUN | Creatinine |
|---|---|---|---|
| PBS | — | 15 | 0.4 |
| ISIS 532800 | 5-10-5 MOE | 26 | 0.5 |
| ISIS 532809 | 5-10-5 MOE | 18 | 0.5 |
| ISIS 588540 | 5-10-5 MOE | 22 | 0.5 |
| ISIS 599268 | 3-10-5 MOE | 28 | 0.5 |
| ISIS 599322 | 6-7-6 MOE | 24 | 0.5 |
| ISIS 599374 | 5-9-5 MOE | 29 | 0.5 |
| ISIS 599378 | 5-9-5 MOE | 22 | 0.4 |
| ISIS 599380 | 5-9-5 MOE | 26 | 0.5 |
| ISIS 599441 | 6-8-6 MOE | 24 | 0.4 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 190
Weights (g)

| | Chemistry | Body | Liver | Spleen | Kidney |
|---|---|---|---|---|---|
| PBS | — | 502 | 16 | 0.9 | 3.7 |
| ISIS 532800 | 5-10-5 MOE | 376 | 16 | 2.0 | 3.4 |
| ISIS 532809 | 5-10-5 MOE | 430 | 16 | 1.4 | 3.4 |
| ISIS 588540 | 5-10-5 MOE | 391 | 16 | 1.8 | 3.5 |
| ISIS 599268 | 3-10-5 MOE | 332 | 16 | 3.6 | 3.6 |
| ISIS 599322 | 6-7-6 MOE | 348 | 13 | 2.1 | 3.4 |
| ISIS 599374 | 5-9-5 MOE | 302 | 12 | 2.0 | 3.3 |
| ISIS 599378 | 5-9-5 MOE | 332 | 11 | 1.1 | 2.8 |
| ISIS 599380 | 5-9-5 MOE | 350 | 11 | 1.5 | 3.3 |
| ISIS 599441 | 6-8-6 MOE | 368 | 16 | 2.5 | 3.3 |

Study 5 (with MOE Gapmers and Deoxy, MOE and (S)-cEt Oligonucleotides)

Male Sprague-Dawley rats, nine- to ten-week old, were maintained on a 12-hour light/dark cycle and fed ad libitum with Purina normal rat chow, diet 5001. Groups of 4 Sprague-Dawley rats each were injected subcutaneously once a week for 6 weeks with 100 mg/kg of MOE gapmer or with 50 mg/kg of deoxy, MOE and (S)-cEt oligonucleotides. One control group of 4 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L. ISIS oligonucleotides that caused changes in the levels of any markers of liver function outside the expected range for antisense oligonucleotides were excluded in further studies.

TABLE 191
Liver function markers in Sprague-Dawley rats

| | | ALT | AST | Albumin |
|---|---|---|---|---|
| | Chemistry | (IU/L) | (IU/L) | (g/dL) |
| PBS | — | 49 | 74 | 3.3 |
| ISIS 532770 | 5-10-5 MOE | 95 | 132 | 3.3 |
| ISIS 588851 | Deoxy, MOE, and (S)-cEt | 47 | 72 | 3.1 |
| ISIS 588856 | Deoxy, MOE, and (S)-cEt | 56 | 75 | 3.0 |
| ISIS 588865 | Deoxy, MOE, and (S)-cEt | 62 | 84 | 2.9 |
| ISIS 588867 | Deoxy, MOE, and (S)-cEt | 73 | 214 | 2.9 |
| ISIS 588868 | Deoxy, MOE, and (S)-cEt | 59 | 83 | 3.1 |
| ISIS 588870 | Deoxy, MOE, and (S)-cEt | 144 | 144 | 3.4 |

### Kidney Function

To evaluate the effect of ISIS oligonucleotides on kidney function, plasma and urine levels of blood urea nitrogen (BUN) and creatinine were measured using an automated clinical chemistry analyzer (Hitachi Olympus AU400e, Melville, NY). Results are presented in the Tables below, expressed in mg/dL. ISIS oligonucleotides that caused changes in the levels of any of the kidney function markers outside the expected range for antisense oligonucleotides were excluded in further studies.

TABLE 192
Kidney function markers (mg/dL) in
the plasma of Sprague-Dawley rats

| | Chemistry | BUN | Creatinine |
|---|---|---|---|
| PBS | — | 18 | 0.3 |
| ISIS 532770 | 5-10-5 MOE | 20 | 0.4 |
| ISIS 588851 | Deoxy, MOE, and (S)-cEt | 20 | 0.4 |
| ISIS 588856 | Deoxy, MOE, and (S)-cEt | 22 | 0.4 |
| ISIS 588865 | Deoxy, MOE, and (S)-cEt | 24 | 0.5 |
| ISIS 588867 | Deoxy, MOE, and (S)-cEt | 22 | 0.4 |
| ISIS 588868 | Deoxy, MOE, and (S)-cEt | 19 | 0.4 |
| ISIS 588870 | Deoxy, MOE, and (S)-cEt | 20 | 0.5 |

TABLE 193
Kidney function markers (mg/dL) in
the urine of Sprague-Dawley rats

| | Chemistry | Total protein | Creatinine |
|---|---|---|---|
| PBS | — | 80 | 92 |
| ISIS 532770 | 5-10-5 MOE | 466 | 69 |
| ISIS 588851 | Deoxy, MOE, and (S)-cEt | 273 | 64 |
| ISIS 588856 | Deoxy, MOE, and (S)-cEt | 259 | 68 |
| ISIS 588865 | Deoxy, MOE, and (S)-cEt | 277 | 67 |
| ISIS 588867 | Deoxy, MOE, and (S)-cEt | 337 | 68 |
| ISIS 588868 | Deoxy, MOE, and (S)-cEt | 326 | 75 |
| ISIS 588870 | Deoxy, MOE, and (S)-cEt | 388 | 82 |

### Weights

Body weight measurements were taken on day 39. Liver, heart, spleen and kidney weights were measured at the end of the study on day 42, and are presented in the Table below. ISIS oligonucleotides that caused any changes in organ weights outside the expected range for antisense oligonucleotides were excluded from further studies.

TABLE 194
Weights (g)

| | Chemistry | Body | Liver | Spleen | Kidney |
|---|---|---|---|---|---|
| PBS | — | 489 | 16 | 0.9 | 3.5 |
| ISIS 532770 | 5-10-5 MOE | 372 | 15 | 1.7 | 3.1 |
| ISIS 588851 | Deoxy, MOE, and (S)-cEt | 285 | 14 | 1.4 | 3.2 |
| ISIS 588856 | Deoxy, MOE, and (S)-cEt | 415 | 15 | 1.1 | 3.3 |
| ISIS 588865 | Deoxy, MOE, and (S)-cEt | 362 | 14 | 2.0 | 3.3 |
| ISIS 588867 | Deoxy, MOE, and (S)-cEt | 406 | 15 | 2.4 | 3.4 |
| ISIS 588868 | Deoxy, MOE, and (S)-cEt | 399 | 15 | 1.5 | 3.4 |
| ISIS 588870 | Deoxy, MOE, and (S)-cEt | 446 | 14 | 1.4 | 3.3 |

Study 6 (with MOE Gapmers, Deoxy, MOE and (S)-cEt Oligonucleotides, and (S)-cEt Gapmers)

Male rats were maintained on a 12-hour light/dark cycle and fed ad libitum with Purina normal rat chow, diet 5001. Groups of 4 rats each were injected subcutaneously once a week for 6 weeks with 100 mg/kg of MOE gapmers or with 50 mg/kg of deoxy, MOE and (S)-cEt oligonucleotide or (S)-cEt gapmer. One control group of 4 rats was injected subcutaneously once a week for 6 weeks with PBS. Forty eight hours after the last dose, rats were euthanized and organs and plasma were harvested for further analysis.

### Liver Function

To evaluate the effect of ISIS oligonucleotides on hepatic function, plasma levels of transaminases were measured on day 42 using an automated clinical chemistry analyzer (Hitachi Olympus AU400e, Melville, NY). Plasma levels of ALT (alanine transaminase) and AST (aspartate transaminase) were measured and the results are presented in the Table below expressed in IU/L.

TABLE 195
Liver function markers

| | | Dose | ALT | AST | Albumin |
|---|---|---|---|---|---|
| | Chemistry | (mg/kg/wk) | (IU/L) | (IU/L) | (g/dL) |
| PBS | — | — | 54 | 73 | 4.3 |
| ISIS 532770 | 5-10-5 MOE | 100 | 57 | 114 | 4.4 |
| ISIS 532800 | 5-10-5 MOE | 100 | 176 | 180 | 4.3 |
| ISIS 532809 | 5-10-5 MOE | 100 | 71 | 132 | 4.1 |
| ISIS 588540 | 5-10-5 MOE | 100 | 89 | 202 | 4.4 |
| ISIS 588544 | 5-10-5 MOE | 100 | 75 | 152 | 3.9 |
| ISIS 588548 | 5-10-5 MOE | 100 | 50 | 71 | 4.1 |
| ISIS 588550 | 5-10-5 MOE | 100 | 80 | 133 | 3.6 |
| ISIS 588553 | 5-10-5 MOE | 100 | 59 | 112 | 3.9 |
| ISIS 588555 | 5-10-5 MOE | 100 | 97 | 142 | 3.8 |
| ISIS 588848 | Deoxy, MOE | 50 | 53 | 82 | 3.9 |
| | and (S)-cEt | | | | |
| ISIS 594430 | 3-10-3 (S)-cEt | 50 | 198 | 172 | 4.4 |

### Kidney Function

To evaluate the effect of ISIS oligonucleotides on kidney function, urine levels of total protein and creatinine were measured using an automated clinical chemistry analyzer (Hitachi Olympus AU400e, Melville, NY). Results are presented in the Table below. ISIS oligonucleotides that caused changes in the levels of any of the kidney function markers outside the expected range for antisense oligonucleotides were excluded in further studies.

TABLE 196
Total protein/creatinine ratio in the urine of rats

| | Chemistry | Dose (mg/kg/wk) | P/C ratio |
|---|---|---|---|
| PBS | — | — | 1.1 |
| ISIS 532770 | 5-10-5 MOE | 100 | 8.3 |
| ISIS 532800 | 5-10-5 MOE | 100 | 6.5 |
| ISIS 532809 | 5-10-5 MOE | 100 | 6.1 |
| ISIS 588540 | 5-10-5 MOE | 100 | 10.1 |
| ISIS 588544 | 5-10-5 MOE | 100 | 7.9 |
| ISIS 588548 | 5-10-5 MOE | 100 | 6.6 |
| ISIS 588550 | 5-10-5 MOE | 100 | 7.6 |
| ISIS 588553 | 5-10-5 MOE | 100 | 7.0 |
| ISIS 588555 | 5-10-5 MOE | 100 | 6.2 |
| ISIS 588848 | Deoxy, MOE | 50 | 5.2 |
| | and (S)-cEt | | |
| ISIS 594430 | 3-10-3 (S)-cEt | 50 | 5.3 |

### Weights

Body weight measurements were taken on day 39. Liver, heart, spleen and kidney weights were measured at the end of the study on day 42, and are presented in the Table below. The results for the organ weights were expressed as a ratio to the body weights and normalized to the PBS control ratio.

TABLE 197
Organ weights/Body weight (BW) ratios

| | | Dose | Spleen/ | Liver/ | Kidney/ |
|---|---|---|---|---|---|
| | Chemistry | (mg/kg/wk) | BW | BW | BW |
| PBS | — | — | 1.0 | 1.0 | 1.0 |
| ISIS 532770 | 5-10-5 MOE | 100 | 2.0 | 1.2 | 1.0 |
| ISIS 532800 | 5-10-5 MOE | 100 | 2.8 | 1.3 | 1.0 |
| ISIS 532809 | 5-10-5 MOE | 100 | 2.2 | 1.1 | 1.0 |
| ISIS 588540 | 5-10-5 MOE | 100 | 2.2 | 1.4 | 1.0 |
| ISIS 588544 | 5-10-5 MOE | 100 | 2.5 | 1.3 | 1.1 |
| ISIS 588548 | 5-10-5 MOE | 100 | 2.1 | 1.3 | 1.1 |
| ISIS 588550 | 5-10-5 MOE | 100 | 3.9 | 1.4 | 1.1 |
| ISIS 588553 | 5-10-5 MOE | 100 | 4.1 | 1.4 | 1.4 |
| ISIS 588555 | 5-10-5 MOE | 100 | 1.8 | 1.3 | 1.0 |
| ISIS 588848 | Deoxy, MOE | 50 | 3.1 | 1.3 | 1.1 |
| | and (S)-cEt | | | | |
| ISIS 594430 | 3-10-3 (S)-cEt | 50 | 1.7 | 1.0 | 1.1 |

### Example 128: Efficacy of Antisense Oligonucleotides Against CFB mRNA in hCFB Mice

Selected compounds were tested for efficacy in human CFB transgenic mice, founder line 46 The human CFB gene is located on chromosome 6: position 31913721-31919861. A Fosmid (ABC14-50933200C23) containing the CFB sequence was selected to make transgenic mice expressing the human CFB gene. Cla I (31926612) and Age 1 (31926815) restriction enzymes were used to generate a 22,127 bp fragment containing the CFB gene for pronuclear injection. DNA was confirmed by restriction enzyme analysis using Pvu I. The 22,127 bp DNA fragment was injected into C57BL/6NTac embryos. 6 positive founders were bred. Founder #6 expressed the liver human CFB mRNA and was crossbreed to the 3′ generation. Progeny from 3^{rd} generation mice were used to evaluate human CFB ASOs for human CFB mRNA reduction.

### Treatment

Groups of 3 mice each were injected subcutaneously twice a week for the first week with 50 mg/kg of ISIS oligonucleotides, followed by once a week dosing with 50 mg/kg of ISIS oligonucleotides for an additional three weeks. One control group of 4 mice was injected subcutaneously twice a week for 2 weeks for the first week with PBS for the first week for an additional three weeks. Forty eight hours after the last dose, mice were euthanized and organs and plasma were harvested for further analysis.

### RNA Analysis

At the end of the dosing period, RNA was extracted from the liver and kidney for real-time PCR analysis of CFB mRNA levels. Human CFB mRNA levels were measured using the human primer probe set RTS3459. CFB mRNA levels were normalized to RIBOGREEN®, and also to the housekeeping gene, Cyclophilin. Results were calculated as percent inhibition of CFB mRNA expression compared to the control. All the antisense oligonucleotides effected inhibition of human CFB mRNA levels in the liver.

TABLE 198
Percent reduction of CFB
mRNA levels in hCFB mice

| | Normalized | Normalized |
|---|---|---|
| | to | to |
| ISIS No | RIBOGREEN | Cyclophilin |
| 532770 | 86 | 87 |
| 532800 | 88 | 87 |
| 532809 | 69 | 69 |
| 588540 | 95 | 94 |
| 588544 | 91 | 91 |
| 588548 | 78 | 77 |
| 588550 | 89 | 88 |
| 588553 | 94 | 94 |
| 588555 | 94 | 94 |
| 588848 | 83 | 82 |
| 594430 | 78 | 76 |

### Example 129: In Vivo Antisense Inhibition of Murine CFB

Several antisense oligonucleotides were designed that were targeted to murine CFB mRNA (GENBANK Accession No. NM_008198.2, incorporated herein as SEQ ID NO: 5). The target start sites and sequences of each oligonucleotide are described in the table below. The chimeric antisense oligonucleotides in the table below were designed as 5-10-5 MOE gapmers. The gapmers are 20 nucleosides in length, wherein the central gap segment is comprised of 102′-deoxynucleosides and is flanked on both sides (in the 5′ and 3′ directions) by wings comprising 5 nucleosides each. Each nucleoside in the 5′ wing segment and each nucleoside in the 3′ wing segment has a 2′-MOE modification. The internucleoside linkages throughout each gapmer are phosphorothioate (P═S) linkages. All cytosine residues throughout each gapmer are 5-methylcytosines.

TABLE 199
Gapmers targeting murine CFB

| | | Target Start | |
|---|---|---|---|
| ISIS | | Site on SEQ | SEQ ID |
| No | Sequence | ID NO: 5 | NO |
| 516269 | GCATAAGAGGGTACCAGCTG | 2593 | 804 |
| 516272 | GTCCTTTAGCCAGGGCAGCA | 2642 | 805 |
| 516323 | TCCACCCATGTTGTGCAAGC | 1568 | 806 |
| 516330 | CCACACCATGCCACAGAGAC | 1826 | 807 |
| 516341 | TTCCGAGTCAGGCTCTTCCC | 2308 | 808 |

### Treatment

Groups of four C57BL/6 mice each were injected with 50 mg/kg of ISIS 516269, ISIS 516272, ISIS 516323, ISIS 516330, or ISIS 516341 administered weekly for 3 weeks. A control group of mice was injected with phosphate buffered saline (PBS) administered weekly for 3 weeks.

### CFB RNA Analysis

At the end of the study, RNA was extracted from liver tissue for real-time PCR analysis of CFB, using primer probe set RTS3430 (forward sequence GGGCAAACAGCAATTTGTGA, designated herein as SEQ ID NO: 816; reverse sequence TGGCTACCCACCTTCCTTGT, designated herein as SEQ ID NO: 817; probe sequence CTGGATACTGTCCCAATCCCGGTATTCCX, designated herein as SEQ ID NO: 818). The mRNA levels were normalized using RIBOGREEN®. As shown in the Table below, some of the antisense oligonucleotides achieved reduction of murine CFB over the PBS control. Results are presented as percent inhibition of CFB, relative to control.

TABLE 200
Percent inhibition of murine
CFB mRNA in C57BL/6 mice

| ISIS No | % |
|---|---|
| 516269 | 29 |
| 516272 | 72 |
| 516323 | 77 |
| 516330 | 62 |
| 516341 | 72 |

### Protein Analysis

CFB protein levels were measured in the kidney, liver, plasma, and in the eye by western Blot using goat anti-CFB antibody (Sigma Aldrich). Results are presented as percent inhibition of CFB, relative to PBS control. ‘n/a’ indicates that measurements were not taken for that sample. As shown in the Table below, antisense inhibition of CFB by ISIS oligonucleotides resulted in a reduction of CFB protein in various tissues. As shown in the Table below, systemic administration of ISIS oligonucleotides was effective in reducing CFB levels in the eye.

TABLE 201
Percent inhibition of murine
CFB protein in C57BL/6 mice

| ISIS No | Kidney | Liver | Plasma | Eye |
|---|---|---|---|---|
| 516269 | 20 | 58 | n/a | 70 |
| 516272 | 48 | 74 | n/a | 99 |
| 516323 | 73 | 85 | 90 | 93 |
| 516330 | 77 | 80 | n/a | n/a |
| 516341 | 80 | 88 | 68 | n/a |

### Example 130: Dose-Dependent Antisense Inhibition of Murine CFB

Groups of four C57BL/6 mice each were injected with 25 mg/kg, 50 mg/kg, or 100 mg/kg of ISIS 516272, and ISIS 516323 administered weekly for 6 weeks. Another two groups of mice were injected with 100 mg/kg of ISIS 516330 or ISIS 516341 administered weekly for 6 weeks. Two control groups of mice were injected with phosphate buffered saline (PBS) administered weekly for 6 weeks.

### CFB RNA Analysis

RNA was extracted from liver and kidney tissues for real-time PCR analysis of CFB, using primer probe set RTS3430. The mRNA levels were normalized using RIBOGREEN®. As shown in the Table below, the antisense oligonucleotides achieved dose-dependent reduction of murine CFB over the PBS control. Results are presented as percent inhibition of CFB, relative to control.

TABLE 202
Percent inhibition of murine
CFB mRNA in C57BL/6 mice

| | Dose | | |
|---|---|---|---|
| ISIS No | (mg/kg/wk) | Liver | Kidney |
| 516272 | 25 | 39 | 32 |
| | 50 | 73 | 36 |
| | 100 | 87 | 42 |
| 516323 | 25 | 36 | 41 |
| | 50 | 65 | 47 |
| | 100 | 79 | 71 |
| 516330 | 100 | 85 | 45 |
| 516341 | 200 | 89 | 65 |

### Protein Analysis

CFB protein levels were measured in the plasma by western Blot using goat anti-CFB antibody (Sigma Aldrich). As shown in the table below, antisense inhibition of CFB by the ISIS oligonucleotides resulted in a reduction of CFB protein. Results are presented as percent inhibition of CFB, relative to PBS control. ‘n/a’ indicates that measurements were not taken for that sample.

CFB protein levels were also measured in the eye by Western Blot. All treatment groups demonstrated an inhibition of CFB by 95%, with some sample measurements being below detection levels of the assay.

TABLE 203
Percent inhibition of murine
CFB protein in C57BL/6 mice

| | Dose | |
|---|---|---|
| ISIS No | (mg/kg/wk) | Liver |
| 516272 | 25 | 32 |
| | 50 | 70 |
| | 100 | 83 |
| 516323 | 25 | 43 |
| | 50 | 80 |
| | 100 | 90 |
| 516330 | 100 | n/a |
| 516341 | 200 | n/a |

### Example 131: Effect of Antisense Inhibition of CFB in the NZB/W F1 Mouse Model

The NZB/W F1 is the oldest classical model of lupus, where the mice develop severe lupus-like phenotypes comparable to that of lupus patients (Theofilopoulos, A. N. and Dixon, F. J. Advances in Immunology, vol. 37, pp. 269-390, 1985). These lupus-like phenotypes include lymphadenopathy, splenomegaly, elevated serum antinuclear autoantibodies (ANA) including anti-dsDNA IgG, a majority of which are IgG2a and IgG3, and immune complex-mediated glomerulonephritis (GN) that becomes apparent at 5-6 months of age, leading to kidney failure and death at 10-12 months of age.

### Study 1

A study was conducted to demonstrate that treatment with antisense oligonucleotides targeting CFB would improve renal pathology in the mouse model. Female NZB/W F1 mice, 17 weeks old, were purchased from Jackson Laboratories. Groups of 16 mice each received doses of 100 μg/kg/week of ISIS 516272 or ISIS 516323 for 20 weeks. Another group of 16 mice received doses of 100 μg/kg/week of control oligonucleotide ISIS 141923 for 20 weeks. Another group of 10 mice received doses of PBS for 20 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. The mRNA levels were normalized using RIBOGREEN®. As shown in the Table below, some of the antisense oligonucleotides achieved reduction of murine CFB over the PBS control. Results are presented as percent inhibition of CFB, relative to control.

TABLE 204
Percent inhibition of murine
CFB mRNA in NZB/W F1 mice

| ISIS No | Liver | Kidney |
|---|---|---|
| 516272 | 55 | 25 |
| 516323 | 63 | 43 |
| 141923 | 0 | 0 |

### Proteinuria

Proteinuria is expected in 60% of animals in this mouse model. The cumulative incidence of severe proteinuria was measured by calculating the total protein to creatinine ratio using a clinical analyzer. The results are presented in the table below and demonstrate that treatment with antisense oligonucleotides targeting CFB achieved reduction of proteinuria in the mice compared to the PBS control and the control oligonucleotide treated mice.

TABLE 205
Percent cumulative incidence of severe
proteinuria in NZB/W F1 mice

| | % |
|---|---|
| PBS | 40 |
| ISIS 516272 | 6 |
| ISIS 516323 | 0 |
| ISIS 141923 | 25 |

### Survival

Survival of the mice was monitored by keeping count of the mice at the start of treatment and then again at week 20. The results are presented in the table below and demonstrate that treatment with antisense oligonucleotides targeting CFB increased survival in the mice compared to the PBS control and the control oligonucleotide treated mice.

TABLE 206
Number of surviving mice and % survival

| | Week | Week | % survival |
|---|---|---|---|
| | 1 | 20 | at week 20 |
| PBS | 10 | 6 | 60 |
| ISIS 516272 | 16 | 15 | 94 |
| ISIS 516323 | 16 | 16 | 100 |
| ISIS 141923 | 16 | 12 | 75 |

### Glomerular Deposition

The amount of C3 deposition, as well as IgG deposition, in the glomeruli of the kidneys was measured by immunohistochemistry with an anti-C3 antibody. The results are presented in the table below and demonstrate that treatment with antisense oligonucleotides targeting CFB achieved reduction of both C3 and IgG depositions in the kidney glomeruli compared to the PBS control and the control oligonucleotide treated mice.

TABLE 207
Percent inhibition of glomerula
deposition in NZB/W F1 mice

| ISIS No | C3 | IgG |
|---|---|---|
| 516272 | 45 | 20 |
| 516323 | 48 | 2 |
| 141923 | 0 | 0 |

### Study 2

Female NZB/W F1 mice, 16 weeks old, were purchased from Jackson Laboratories. A group of 10 mice received doses of 100 μg/kg/week of ISIS 516323 for 12 weeks. Another group of 10 mice received doses of 100 μg/kg/week of control oligonucleotide ISIS 141923 for 12 weeks. Another group of 10 mice received doses of PBS for 12 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. As shown in the table below, treatment with ISIS 516323 achieved reduction of murine CFB over the PBS control. Results are presented as percent inhibition of CFB, relative to control.

TABLE 208
Percent inhibition of murine
CFB mRNA in NZB/W F1 mice

| ISIS No | Liver | Kidney |
|---|---|---|
| 516323 | 75 | 46 |
| 141923 | 0 | 6 |

### Proteinuria

The cumulative incidence of severe proteinuria was assessed by measuring urine total protein to creatinine ratio, as well as by measuring total microalbumin levels. The results are presented in the tables below and demonstrate that treatment with antisense oligonucleotides targeting CFB reduced proteinuria in the mice compared to the PBS control and the control oligonucleotide treated mice.

TABLE 209
Proteinuria in NZB/W F1 mice measured
as urine microalbumin levels (mg/dl)

| ISIS No | Week 0 | Week 6 | Week 8 | Week 10 |
|---|---|---|---|---|
| 516323 | 0 | 0 | 5.4 | 0.4 |
| 141923 | 0 | 8.28 | 8.6 | 5.6 |

TABLE 210
Proteinuria in NZB/W F1 mice measured
as total protein to creatinine ratio

| ISIS No | Week 0 | Week 6 | Week 8 | Week 10 |
|---|---|---|---|---|
| 516323 | 5.5 | 7.8 | 8.6 | 7.2 |
| 141923 | 6.9 | 10.0 | 13.5 | 7.2 |

### Survival

Survival of the mice was monitored by keeping count of the mice at the start of treatment and then again at week 12. The results are presented in the table below and demonstrate that treatment with antisense oligonucleotides targeting CFB increased survival in the mice compared to the PBS control and the control oligonucleotide treated mice.

TABLE 211
Number of surviving mice

| | Week 1 | Week 12 |
|---|---|---|
| PBS | 10 | 9 |
| ISIS 516323 | 10 | 10 |
| ISIS 141923 | 10 | 9 |

### Example 132: Effect of Antisense Inhibition of CFB in the MRL Mouse Model

The MRL/lpr lupus nephritis mouse model develops an SLE-like phenotype characterized by lymphadenopathy due to an accumulation of double negative (CD4⁻ CD8⁻) and B220⁺ T-cells. These mice display an accelerated mortality rate. In addition, the mice have high concentrations of circulating immunoglobulins, which included elevated levels of autoantibodies such as ANA, anti-ssDNA, anti-dsDNA, anti-Sm, and rheumatoid factors, resulting in large amounts of immune complexes (Andrews, B. et al., J. Exp. Med. 148: 1198-1215, 1978).

### Treatment

A study was conducted to investigate whether treatment with antisense oligonucleotides targeting CFB would reverse renal pathology in the mouse model. Female MRL/lpr mice, 14 weeks old, were purchased from Jackson Laboratories. A group of 10 mice received doses of 50 μg/kg/week of ISIS 516323 for 7 weeks. Another group of 10 mice received doses of 50 μg/kg/week of control oligonucleotide ISIS 141923 for 7 weeks. Another group of 10 mice received doses of PBS for 7 weeks and served as the control group to which all the other groups were compared. Terminal endpoints were collected 48 hours after the last dose was injected.

### CFB RNA Analysis

RNA was extracted from liver tissue for real-time PCR analysis of CFB, using primer probe set RTS3430. As shown in the Table below, ISIS 516323 reduced CFB over the PBS control. Results are presented as percent inhibition of CFB, relative to control.

TABLE 212
Percent inhibition of murine
CFB mRNA in MRL/lpr mice

| ISIS No | % |
|---|---|
| 516323 | 68 |
| 141923 | 4 |

### Renal Pathology

Renal pathology was evaluated by two methods. Histological sections of the kidney were stained with Haematoxylin &Eosin. The PBS control demonstrated presence of multiglomerular crescents tubular casts, which is a symptom of glomerulosclerosis. In contrast, the sections from mice treated with ISIS 516323 showed absent crescents tubular casts with minimal bowman capsule fibrotic changes, moderate to severe segmental mesangial cell expansion and glomerular basement membrane thickening.

Accumulation of C3 in the kidney was also assessed by immunohistochemistry with anti-C3 antibodies. The whole kidney C3 immunohistochemistry intensity score was calculated by intensity scoring system, which was computed by capturing 10 glomeruli per kidney and calculation the intensity of positive C3 staining. The results are presented in the table below and demonstrate that treatment with ISIS 516323 reduced renal C3 accumulation compared to the control groups.

TABLE 213
Renal C3 accumulation in MRL/lpr mice

| | Whole kidney C3 | C3 quantification (area/ |
|---|---|---|
| | intensity score | total area) % of average PBS |
| PBS | 2.5 | 100 |
| ISIS 516323 | 1.6 | 68 |
| ISIS 141923 | 2.2 | 99 |

### Plasma C3 Levels

Reduction of CFB inhibits activation of the alternative complement pathway, preventing C3 consumption and leading to an apparent elevation of plasma C3 levels. Plasma C3 levels from terminal bleed were measured by clinical analyzer. The results are presented in the table below and demonstrate that treatment with ISIS 516323 increased C3 levels (p<0.001) in the plasma compared to the control groups.

TABLE 214
Plasma C3 levels (mg/dL) in MRL/lpr mice

| ISIS No | C3 |
|---|---|
| 516323 | 28 |
| 141923 | 16 |

The results indicate that treatment with antisense oligonucleotides targeting CFB reverses renal pathology in the lupus mouse model.

### Example 133: Effect of Antisense Inhibition of CFB in the CFH Het Mouse Model

CFH heterozygous (CFH Het, CFH^{+/-}) mouse model express a mutant Factor H protein in combination with the full-length mouse protein (Pickering, M. C. et al., J. Exp. Med. 2007. 204: 1249-56). Renal histology remains normal in these mice up to six months old.

### Study 1

Groups of 8 CFH^{+/-} mice, 6 weeks old, each received doses of 75 mg/kg/week of ISIS 516323 or ISIS 516341 for 6 weeks. Another group of 8 mice received doses of 75 mg/kg/week of control oligonucleotide ISIS 141923 for 6 weeks. Another group of 8 mice received doses of PBS for 6 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. As shown in the Table below, the antisense oligonucleotides reduced CFB over the PBS control. Results are presented as percent inhibition of CFB, relative to control.

TABLE 215
Percent inhibition of murine CFB mRNA in CFH^{+/-} mice

| ISIS No | Liver | Kidney |
|---|---|---|
| 516323 | 80 | 38 |
| 516341 | 90 | 44 |
| 141923 | 0 | 17 |

### Plasma C3 Levels

Reduction of CFB inhibits activation of the alternative complement pathway, preventing C3 consumption and leading to an apparent elevation of plasma C3 levels. Plasma C3 levels from terminal plasma collection were measured by clinical analyzer. The results are presented in the table below and demonstrate that treatment with ISIS 516323 increased C3 to normal levels in the plasma.

TABLE 216
Plasma C3 levels (mg/dL) in CFH^{+/-} mice

| ISIS No | C3 |
|---|---|
| 516323 | 15 |
| 516341 | 17 |
| 141923 | 8 |

### Study 2

Groups of 5 CFH^{+/-} mice each received doses of 12.5 mg/kg/week, 25 mg/kg/week, 50 mg/kg/week, 75 mg/kg/week, or 100 mg/kg/week of ISIS 516323 or ISIS 516341 for 6 weeks. Another group of 5 mice received doses of 75 μg/kg/week of control oligonucleotide ISIS 141923 for 6 weeks. Another group of 5 mice received doses of PBS for 6 weeks and served as the control group to which all the other groups were compared. Terminal endpoints were collected 48 hours after the last dose was injected.

### CFB RNA Analysis

RNA was extracted from liver and kidney tissue for real-time PCR analysis of CFB, using primer probe set RTS3430. As shown in the Table below, the antisense oligonucleotides reduced CFB over the PBS control in a dose dependent manner. Results are presented as percent inhibition of CFB, relative to control.

TABLE 217
Percent inhibition of murine CFB mRNA in
the liver of CFH^{+/-} mice

| ISIS No | Dose (mg/kg/week) | % |
|---|---|---|
| 516323 | 12.5 | 34 |
| | 25 | 51 |
| | 50 | 72 |
| | 75 | 79 |
| | 100 | 92 |
| 516341 | 12.5 | 38 |
| | 25 | 57 |
| | 50 | 89 |
| | 75 | 92 |
| | 100 | 90 |
| 141923 | 75 | 13 |

### Plasma C3 Levels

Reduction of CFB inhibits activation of the alternative complement pathway, preventing C3 consumption and leading to an apparent elevation of plasma C3 levels. Plasma C3 levels from terminal plasma collection were measured by clinical analyzer. The results are presented in the table below and demonstrate that treatment with ISIS oligonucleotides targeting CFB increased C3 levels in the plasma.

TABLE 218
Plasma C3 levels (mg/dL) in CFH^{+/-} mice

| | Dose (mg/kg/week) | C3 |
|---|---|---|
| PBS | — | 10.1 |
| 516323 | 12.5 | 11.4 |
| | 25 | 15.5 |
| | 50 | 17.0 |
| | 75 | 18.3 |
| | 100 | 18.8 |
| 516341 | 12.5 | 12.1 |
| | 25 | 16.3 |
| | 50 | 18.6 |
| | 75 | 22.1 |
| | 100 | 19.1 |
| 141923 | 75 | 8.9 |

### Example 134: Effect of ISIS Antisense Oligonucleotides Targeting Human CFB in Cynomolgus Monkeys

Cynomolgus monkeys were treated with ISIS antisense oligonucleotides selected from studies described in the Examples above. Antisense oligonucleotide efficacy and tolerability, as well as their pharmacokinetic profile in the liver and kidney, were evaluated.

At the time this study was undertaken, the cynomolgus monkey genomic sequence was not available in the National Center for Biotechnology Information (NCBI) database; therefore cross-reactivity with the cynomolgus monkey gene sequence could not be confirmed. Instead, the sequences of the ISIS antisense oligonucleotides used in the cynomolgus monkeys was compared to a rhesus monkey sequence for homology. It is expected that ISIS oligonucleotides with homology to the rhesus monkey sequence are fully cross-reactive with the cynomolgus monkey sequence as well. The human antisense oligonucleotides tested are cross-reactive with the rhesus genomic sequence (GENBANK Accession No. NW_001116486.1 truncated from nucleotides 536000 to 545000, designated herein as SEQ ID NO: 3). The greater the complementarity between the human oligonucleotide and the rhesus monkey sequence, the more likely the human oligonucleotide can cross-react with the rhesus monkey sequence. The start and stop sites of each oligonucleotide targeted to SEQ ID NO: 3 is presented in the Table below. “Start site” indicates the 5′-most nucleotide to which the gapmer is targeted in the rhesus monkey gene sequence. ‘Mismatches’ indicates the number of nucleobases in the human oligonucleotide that are mismatched with the rhesus genomic sequence.

TABLE 219
Antisense oligonucleotides complementary to the
rhesus CFB genomic sequence (SEQ ID NO: 3)

| ISIS | Target | | | SEQ |
|---|---|---|---|---|
| No | Start Site | Mismatches | Chemistry | ID NO |
| 532770 | 6788 | 0 | 5-10-5 MOE | 198 |
| 532800 | 7500 | 0 | 5-10-5 MOE | 228 |
| 532809 | 7614 | 0 | 5-10-5 MOE | 237 |
| 588540 | 7627 | 1 | 5-10-5 MOE | 440 |
| 588544 | 7631 | 1 | 5-10-5 MOE | 444 |
| 588548 | 7635 | 1 | 5-10-5 MOE | 448 |
| 588550 | 7637 | 1 | 5-10-5 MOE | 450 |
| 588553 | 7640 | 1 | 5-10-5 MOE | 453 |
| 588555 | 7643 | 0 | 5-10-5 MOE | 455 |
| 588848 | 7639 | 1 | Deoxy, MOE and cEt | 598 |
| 594430 | 6790 | 0 | 3-10-3 cEt | 549 |

### Treatment

Prior to the study, the monkeys were kept in quarantine for at least a 30 day period, during which the animals were observed daily for general health. The monkeys were 2-4 years old and weighed between 2 and 4 kg. Eleven groups of 4-6 randomly assigned male cynomolgus monkeys each were injected subcutaneously with ISIS oligonucleotide or PBS at four sites on the back in a clockwise rotation (i.e. left, top, right, and bottom), one site per dose. The monkeys were given four loading doses of PBS or 40 mg/kg of ISIS 532800, ISIS 532809, ISIS 588540, ISIS 588544, ISIS 588548, ISIS 588550, ISIS 588553, ISIS 588555, ISIS 588848, or ISIS 594430 for the first week (days 1, 3, 5, and 7), and were subsequently dosed once a week for 12 weeks (days 14, 21, 28, 35, 42, 49, 56, 63, 70, 77, and 84) with PBS or 40 mg/kg of ISIS oligonucleotide. ISIS 532770 was tested in a separate study with similar conditions with two male and two female cynomolgus monkeys in the group.

### Hepatic Target Reduction

### RNA Analysis

On day 86, liver and kidney samples were collected in duplicate (approximately 250 mg each) for CFB mRNA analysis. The samples were flash frozen in liquid nitrogen at necropsy within approximately 10 minutes of sacrifice.

RNA was extracted from liver and kidney for real-time PCR analysis of measurement of mRNA expression of CFB. Results are presented as percent change of mRNA, relative to PBS control, normalized with RIBOGREEN®. RNA levels were also normalized with the house-keeping gene, Cyclophilin A. RNA levels were measured with the primer probe sets RTS3459, described above, or RTS4445_MGB (forward sequence CGAAGAAGCTCAGTGAAATCAA, designated herein as SEQ ID NO: 819; reverse sequence TGCCTGGAGGGCCCTCTT, designated herein as SEQ ID NO: 820; probe sequence AGACCACAAGTTGAAGTC, designated herein as SEQ ID NO: 815).

As shown in the Tables below, treatment with ISIS antisense oligonucleotides resulted in reduction of CFB mRNA in comparison to the PBS control. Analysis of CFB mRNA levels revealed that several of the ISIS oligonucleotides reduced CFB levels in liver and/or kidney. Here ‘0’ indicates that the expression levels were not inhibited. ‘*’ indicates that the oligonucleotide was tested in a separate study with similar conditions.

TABLE 220
Percent inhibition of CFB mRNA in the cynomolgus
monkey liver relative to the PBS control

| ISIS | RTS3459/ | RTS3459/ | RTS445_MGB/ | RTS445_MGB/ |
|---|---|---|---|---|
| No | Cyclophilin A | RIBOGREEN | Cyclophilin A | RIBOGREEN |
| 532770* | 12 | 37 | 24 | 45 |
| 532800 | 54 | 45 | 56 | 46 |
| 588540 | 31 | 27 | 28 | 24 |
| 588548 | 68 | 67 | 68 | 67 |
| 588550 | 53 | 39 | 51 | 37 |
| 588553 | 74 | 59 | 74 | 59 |
| 588555 | 73 | 71 | 71 | 69 |
| 588848 | 9 | 0 | 6 | 0 |
| 594430 | 24 | 26 | 23 | 25 |

TABLE 221
Percent inhibition of CFB mRNA in the cynomolgus
monkey kidney relative to the PBS control

| ISIS | RTS3459/ | RTS3459/ | RTS445_MGB/ | RTS445_MGB/ |
|---|---|---|---|---|
| No | Cyclophilin A | RIBOGREEN | Cyclophilin A | RIBOGREEN |
| 532770* | 34 | 56 | 2 | 31 |
| 532800 | 36 | 30 | 43 | 37 |
| 588540 | 70 | 71 | 67 | 69 |
| 588548 | 83 | 84 | 82 | 83 |
| 588550 | 81 | 77 | 78 | 74 |
| 588553 | 86 | 84 | 86 | 85 |
| 588555 | 32 | 34 | 48 | 50 |
| 588848 | 89 | 91 | 87 | 90 |
| 594430 | 33 | 37 | 19 | 23 |

### Protein Analysis

Approximately 1 mL of blood was collected from all available animals at day 85 and placed in tubes containing the potassium salt of EDTA. The blood samples were placed in wet-ice or Kryorack immediately, and centrifuged (3000 rpm for 10 min at 4° C.) to obtain plasma (approximately 0.4 mL) within 60 minutes of collection. Plasma levels of CFB were measured in the plasma by radial immunodiffusion (RTD), using a polyclonal anti-Factor B antibody. The results are presented in the Table below. ISIS 532770 was tested in a separate study and plasma protein levels were measured on day 91 or 92 in that group.

Analysis of plasma CFB revealed that several ISIS oligonucleotides reduced protein levels in a sustained manner. ISIS 532770, which was tested in a separate study, reduced CFB protein levels on day 91/92 by 50% compared to baseline values. The reduction in plasma CFB protein levels correlates well with liver CFB mRNA level reduction in the corresponding groups of animals.

TABLE 222
Plasma protein levels (% baseline
values) in the cynomolgus monkey

| | Day 1 | Day 30 | Day 58 | Day 72 | Day 86 |
|---|---|---|---|---|---|
| PBS | 113 | 115 | 95 | 83 | 86 |
| ISIS 532800 | 117 | 68 | 52 | 39 | 34 |
| ISIS 532809 | 104 | 121 | 100 | 80 | 71 |
| ISIS 588540 | 108 | 72 | 61 | 40 | 38 |
| ISIS 588544 | 118 | 74 | 53 | 33 | 29 |
| ISIS 588548 | 110 | 41 | 28 | 20 | 16 |
| ISIS 588550 | 104 | 64 | 54 | 38 | 37 |
| ISIS 588553 | 97 | 42 | 35 | 18 | 16 |
| ISIS 588555 | 107 | 35 | 37 | 18 | 18 |
| ISIS 588848 | 116 | 95 | 92 | 69 | 71 |
| ISIS 594430 | 104 | 64 | 59 | 45 | 46 |

### Tolerability Studies

### Body Weight Measurements

To evaluate the effect of ISIS oligonucleotides on the overall health of the animals, body and organ weights were measured and are presented in the Table below. ‘*’ indicates that the oligonucleotide was tested in a separate study with similar conditions and is the average of the measurements from male and female monkeys. The results indicate that effect of treatment with antisense oligonucleotides on body and organ weights was within the expected range for antisense oligonucleotides.

TABLE 223
Final body weights (g) in cynomolgus monkey

| | Day | Day | Day | Day | Day | Day | Day |
|---|---|---|---|---|---|---|---|
| | 1 | 14 | 28 | 42 | 56 | 70 | 84 |
| PBS | 2887 | 2953 | 3028 | 3094 | 3125 | 3143 | 3193 |
| ISIS 532770* | 2963 | 2947 | 2966 | 3050 | 3097 | 3138 | 3160 |
| ISIS 532800 | 2886 | 2976 | 3072 | 3149 | 3220 | 3269 | 3265 |
| ISIS 532809 | 2755 | 2836 | 2927 | 2983 | 3019 | 3071 | 3098 |
| ISIS 588540 | 2779 | 2834 | 2907 | 2934 | 2981 | 3034 | 3057 |
| ISIS 588544 | 2837 | 2896 | 3009 | 3064 | 3132 | 3163 | 3199 |
| ISIS 588548 | 2694 | 2816 | 2882 | 2990 | 3073 | 3149 | 3161 |
| ISIS 588550 | 2855 | 2988 | 3062 | 3188 | 3219 | 3282 | 3323 |
| ISIS 588553 | 3033 | 3156 | 3256 | 3335 | 3379 | 3372 | 3442 |
| ISIS 588555 | 2757 | 2863 | 2965 | 3022 | 3075 | 3088 | 3158 |
| ISIS 588848 | 2850 | 3018 | 3032 | 3187 | 3230 | 3212 | 3291 |
| ISIS 594430 | 2884 | 2963 | 2953 | 3149 | 3187 | 3204 | 3256 |

TABLE 224
Final organ weights (g) in cynomolgus monkey

| | Spleen | Heart | Kidney | Liver |
|---|---|---|---|---|
| PBS | 2.8 | 11.6 | 11.9 | 55.8 |
| ISIS 532770* | 5.0 | 11.3 | 20.6 | 77.9 |
| ISIS 532800 | 6.2 | 11.9 | 18.6 | 94.4 |
| ISIS 588540 | 4.0 | 11.4 | 13.5 | 67.1 |
| ISIS 588548 | 4.1 | 11.7 | 17.3 | 72.0 |
| ISIS 588550 | 5.8 | 10.9 | 18.5 | 81.8 |
| ISIS 588553 | 5.0 | 12.7 | 17.2 | 85.9 |
| ISIS 588555 | 4.7 | 11.8 | 15.9 | 88.3 |
| ISIS 588848 | 5.0 | 12.7 | 14.4 | 75.7 |
| ISIS 594430 | 3.9 | 11.9 | 14.8 | 69.9 |

### Liver Function

To evaluate the effect of ISIS oligonucleotides on hepatic function, blood samples were collected from all the study groups. The blood samples were collected from the cephalic, saphenous, or femoral veins, 48 hours post-dosing. The monkeys were fasted overnight prior to blood collection. Blood (1.5 mL) was collected in tubes without anticoagulant for serum separation. The tubes were kept at room temperature for a minimum of 90 minutes and then centrifuged (approximately 3,000 rpm for 10 min) to obtain serum. Levels of various liver function markers were measured using a Toshiba 200FR NEO chemistry analyzer (Toshiba Co., Japan).

Plasma levels of ALT and AST were measured and the results are presented in the Table below, expressed in IU/L. Bilirubin, a liver function marker, was similarly measured and is presented in the Table below expressed in mg/dL. ‘*’ indicates that the oligonucleotide was tested in a separate study with similar conditions and is the average of the measurements from male and female monkeys. The results indicate that most of the antisense oligonucleotides had no effect on liver function outside the expected range for antisense oligonucleotides.

TABLE 225
Liver chemistry marker levels in cynomolgus
monkey plasma on day 86

| | ALT (IU/L) | AST (IU/L) | Bilirubin (mg/dL) |
|---|---|---|---|
| PBS | 71 | 57 | 0.3 |
| ISIS 532770* | 59 | 58 | 0.1 |
| ISIS 532800 | 65 | 86 | 0.1 |
| ISIS 532809 | 35 | 58 | 0.1 |
| ISIS 588540 | 70 | 88 | 0.2 |
| ISIS 588544 | 55 | 97 | 0.2 |
| ISIS 588548 | 61 | 85 | 0.2 |
| ISIS 588550 | 94 | 84 | 0.2 |
| ISIS 588553 | 44 | 65 | 0.2 |
| ISIS 588555 | 63 | 84 | 0.2 |
| ISIS 588848 | 69 | 65 | 0.2 |
| ISIS 594430 | 86 | 53 | 0.2 |

### Kidney Function

To evaluate the effect of ISIS oligonucleotides on kidney function, blood samples were collected from all the study groups. The blood samples were collected from the cephalic, saphenous, or femoral veins, 48 hours post-dosing. The monkeys were fasted overnight prior to blood collection. Blood was collected in tubes without anticoagulant for serum separation. The tubes were kept at room temperature for a minimum of 90 minutes and then centrifuged (approximately 3,000 rpm for 10 min) to obtain serum. Levels of BUN and creatinine were measured using a Toshiba 200FR NEO chemistry analyzer (Toshiba Co., Japan). Results are presented in the Table below, expressed in mg/dL. ‘*’ indicates that the oligonucleotide was tested in a separate study with similar conditions and is the average of the measurements from male and female monkeys.

For urinalysis, fresh urine from all the animals was collected in the morning using a clean cage pan on wet ice. Food was removed overnight the day before urine collection but water was supplied. Urine samples (approximately 1 mL) were analyzed for protein to creatinine (P/C) ratio using a Toshiba 200FR NEO automated chemistry analyzer (Toshiba Co., Japan). ‘n.d.’ indicates that the urine protein level was under the detection limit of the analyzer.

The plasma and urine chemistry data indicate that most of the ISIS oligonucleotides did not have any effect on the kidney function outside the expected range for antisense oligonucleotides.

TABLE 226
Renal chemistry marker levels (mg/dL)
in cynomolgus monkey plasma on day 86

| | BUN | Creatinine | Total protein |
|---|---|---|---|
| PBS | 28 | 0.9 | 8.0 |
| ISIS 532770* | 20 | 0.9 | 6.9 |
| ISIS 532800 | 25 | 0.9 | 7.5 |
| ISIS 532809 | 23 | 0.8 | 7.4 |
| ISIS 588540 | 30 | 0.8 | 7.5 |
| ISIS 588544 | 26 | 0.9 | 7.4 |
| ISIS 588548 | 25 | 0.9 | 7.6 |
| ISIS 588550 | 24 | 0.9 | 7.2 |
| ISIS 588553 | 25 | 0.8 | 7.2 |
| ISIS 588555 | 25 | 0.8 | 7.6 |
| ISIS 588848 | 24 | 0.9 | 7.5 |
| ISIS 594430 | 25 | 0.8 | 7.2 |

TABLE 227
Renal chemistry marker levels in cynomolgus
monkey urine on day 44 and day 86

| | Day 44 | Day 86 |
|---|---|---|
| PBS | 0.03 | n.d. |
| ISIS 532800 | 0.01 | n.d. |
| ISIS 532809 | 0.01 | n.d. |
| ISIS 588540 | 0.03 | n.d. |
| ISIS 588544 | 0.01 | 0.09 |
| ISIS 588548 | 0.01 | 0.01 |
| ISIS 588550 | 0.04 | 0.01 |
| ISIS 588553 | 0.05 | n.d. |
| ISIS 588555 | 0.03 | 0.03 |
| ISIS 588848 | 0.09 | n.d. |
| ISIS 594430 | 0.03 | n.d. |

### Hematology

To evaluate any effect of ISIS oligonucleotides in cynomolgus monkeys on hematologic parameters, blood samples of approximately 0.5 mL of blood was collected from each of the available study animals in tubes containing K₂-EDTA. Samples were analyzed for red blood cell (RBC) count, white blood cells (WBC) count, individual white blood cell counts, such as that of monocytes, neutrophils, lymphocytes, as well as for platelet count, hemoglobin content and hematocrit, using an ADVIA120 hematology analyzer (Bayer, USA). The data is presented in the Tables below. ‘*’ indicates that the oligonucleotide was tested in a separate study with similar conditions and is the average of the measurements from male and female monkeys.

The data indicate the oligonucleotides did not cause any changes in hematologic parameters outside the expected range for antisense oligonucleotides at this dose.

TABLE 228
Blood cell counts in cynomolgus monkeys

| | RBC | Platelets | WBC | Neutrophils | Lymphocytes | Monocytes |
|---|---|---|---|---|---|---|
| | (×10⁶/μL) | (×10³/μL) | (×10³/μL) | (% WBC) | (% total) | (% total) |
| PBS | 5.8 | 347 | 9.4 | 42.7 | 53.1 | 3.0 |
| ISIS 532770* | 5.4 | 386 | 10.8 | 22.3 | 71.7 | 3.3 |
| ISIS 532800 | 5.6 | 360 | 13.1 | 29.5 | 61.1 | 6.5 |
| ISIS 532809 | 5.2 | 400 | 11.5 | 56.6 | 38.2 | 2.5 |
| ISIS 588540 | 5.5 | 367 | 11.7 | 50.9 | 42.7 | 2.1 |
| ISIS 588544 | 5.2 | 373 | 14.3 | 56.6 | 37.6 | 4.3 |
| ISIS 588548 | 5.1 | 373 | 9.7 | 40.4 | 54.3 | 3.9 |
| ISIS 588550 | 6.1 | 343 | 9.9 | 32.1 | 61.7 | 4.6 |
| ISIS 588553 | 5.2 | 424 | 9.3 | 41.7 | 53.2 | 3.6 |
| ISIS 588555 | 5.1 | 411 | 9.6 | 45.1 | 49.7 | 3.5 |
| ISIS 588848 | 5.7 | 370 | 10.0 | 39.8 | 55.8 | 3.1 |
| ISIS 594430 | 5.7 | 477 | 10.6 | 47.3 | 47.8 | 3.6 |

TABLE 229
Hematologic parameters in cynomolgus monkeys

| | Hemoglobin (g/dL) | HCT (%) |
|---|---|---|
| PBS | 14.1 | 46.6 |
| ISIS 532770* | 12.4 | 40.9 |
| ISIS 532800 | 12.3 | 40.5 |
| ISIS 532809 | 12.2 | 40.4 |
| ISIS 588540 | 12.5 | 41.5 |
| ISIS 588544 | 11.9 | 38.1 |
| ISIS 588548 | 12.3 | 39.6 |
| ISIS 588550 | 13.4 | 45.0 |
| ISIS 588553 | 12.6 | 39.8 |
| ISIS 588555 | 11.6 | 38.1 |
| ISIS 588848 | 13.2 | 42.7 |
| ISIS 594430 | 13.4 | 43.1 |

### Measurement of Oligonucleotide Concentration

The concentration of the full-length oligonucleotide was measured in the kidney and liver tissues. The method used is a modification of previously published methods (Leeds et al., 1996; Geary et al., 1999) which consist of a phenol-chloroform (liquid-liquid) extraction followed by a solid phase extraction. Tissue sample concentrations were calculated using calibration curves, with a lower limit of quantitation (LLOQ) of approximately 1.14 μg/g. The results are presented in the Table below, expressed as μg/g liver or kidney tissue.

TABLE 230
Antisense oligonucleotide distribution

| | Kidney (μg/g) | Liver (μg/g) | Kidney/Liver ratio |
|---|---|---|---|
| ISIS 532800 | 3881 | 1633 | 2.4 |
| ISIS 588540 | 3074 | 1410 | 2.2 |
| ISIS 588548 | 3703 | 1233 | 3.0 |
| ISIS 588550 | 4242 | 860 | 4.9 |
| ISIS 588553 | 3096 | 736 | 4.2 |
| ISIS 588555 | 4147 | 1860 | 2.2 |
| ISIS 588848 | 2235 | 738 | 3.0 |
| ISIS 594430 | 1548 | 752 | 2.1 |

### Example 135: 6 Week Efficacy Study of Unconjugated and 5′-THA-GalNAc3 Conjugated Antisense Oligonucleotides Targeted to Human CFB in Transgenic Mice

Two antisense oligonucleotides having the same nucleobase sequence: unconjugated antisense oligonucleotide ISIS 588540 and 5′-THA-GalNAc3-conjugated antisense oligonucleotide ISIS 696844, were tested in human CFB transgenic mice (hCFB-Tg mice).

The mice were administered subcutaneously with ISIS 696844 at doses of 0.1, 1.25, 0.5, 2.0, 6.0, or 12.0 mg/kg/week or with ISIS 588540 at doses of 2, 6, 12, 25, or 50 mg/kg/week for 6 weeks. A control group of mice were administered subcutaneously with PBS for 6 weeks. Mice were sacrificed 48 hours after the last dose. Hepatic mRNA levels were analyzed by qRT-PCR.

### Study 1

The results are presented in the Table below and demonstrate that the 5′-THA-GalNAc3-conjugated antisense oligonucleotide targeting CFB is more potent than the unconjugated antisense oligonucleotide with the same sequence.

TABLE 231
Efficacy of antisense oligonucleotides targeting CFB

| | ED₅₀ (mg/kg) | ED₇₅ (mg/kg) |
|---|---|---|
| ISIS 588540 | 4.52 | 9.26 |
| ISIS 696844 | 0.52 | 1.12 |

### Study 2

Liver mRNA levels were measured with two different primer probe sets targeting different regions of the mRNA and normalized to either RIBOGREEN® (RGB) or Cyclophilin. The primer probe sets were RTS3459, described above, and RTS3460 (forward sequence CGAAGCAGCTCAATGAAATCAA, designated herein as SEQ ID NO: 813; reverse sequence TGCCTGGAGGGCCTTCTT, designated herein as SEQ ID NO: 814; probe sequence AGACCACAAGTTGAAGTC, designated herein as SEQ ID NO: 815). The results are presented in the Table below and demonstrate that the 5′-THA-GalNAc3-conjugated antisense oligonucleotide targeting CFB is more potent than the unconjugated antisense oligonucleotide with the same sequence, irrespective of the primer probe set used.

TABLE 231
Efficacy of antisense oligonucleotides targeting CFB

| | ED₅₀ | ED₅₀ | ED₅₀ | ED₅₀ | ED₇₅ | ED₇₅ | ED₇₅ | ED₇₅ |
|---|---|---|---|---|---|---|---|---|
| | RTS3459 | RTS3460 | RTS3459 | RTS3460 | RTS3459 | RTS3460 | RTS3459 | RTS3460 |
| | (RGB) | (RGB) | (Cyclophilin) | (Cyclophilin) | (RGB) | (RGB) | (Cyclophilin) | (Cyclophilin) |
| ISIS 588540 | 4.5 | 4.1 | 5.2 | 5.4 | 9.3 | 10.0 | 10.0 | 9.3 |
| ISIS 696844 | 0.5 | 0.5 | 0.6 | 0.5 | 1.1 | 1.3 | 1.2 | 0.9 |

1-263. (canceled)

264. A compound comprising a modified oligonucleotide and a conjugate group, wherein the modified oligonucleotide consists of 10 to 30 linked nucleosides having a nucleobase sequence comprising any one of SEQ ID NOs: 198, 228, 237, 440, 444, 448, 450, 453, 455, 549, and 598, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment and
wherein each nucleoside of each wing segment comprises a modified sugar.


SEQUENCE LISTING (854 sequences)

SEQ ID NO: 1 - DNA, 2646 bp, Homo sapiens
  source 1..2646: mol_type = genomic DNA
gacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacaaaaaaaaaaaaaaaaaaaa

SEQ ID NO: 2 - DNA, 9001 bp, Homo sapiens
  source 1..9001: mol_type = genomic DNA
ggaggtgagggtctcaggttggggatgctgggatccccctgtgacagctcccagaatgtctctcttccttctccaggtctggctgctttctctctctgacgcgggtcacccctcctcccaagcctcacaaacctgctaggtgtccctgggtctgcttattctttttttgttgttattgagatggagtcttgctctgtctcccaggctggagtgcagtggcacgacctcagctcactgcaacttctgcctcctgggttcaagcgattctcctacttcagcctcccgagtagctgagattacaggtgcccaccaccacaccagctaatttttgtatttttagtagagacgggatttcgccatgttggccaggatggtcttgaactcctgacctcaagtgatctgcctgcctcaacctcccaaagtgctgagattacaggcgtgagccactgcacccacccgggtctgcttattctacccttctctctggttccacccctgctgcagtggacaagctgtgccgaggttgtctcccaagaaaaaaccatgttccccaacttgacagatgtcagggaggtggtgacagaccagttcctatgcagtgggacccaggaggatgagagtccctgcaagggtgagtccctcaccatgcctggattcccaaggggaaggccacctgtgtctctgtggccagcatgcatgccagaacaccagtccactgccctagatgacactgtctcctgtcaccctttgctggcaggagaatctgggggagcagttttccttgagcggagattcaggttttttcaggtgagaaggtagaagcttgcaggacccaggggttacaggatctcagccttgttggggggatgagggaggcctttgagggatctagggaggttggggcttacagttggggctgtggcagcctcccagccagttctctccttttctccaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccacatctctcagtgcctctatctggaggccaggtagggctggccttgggggagggggaggccagaatgactccaagagctacaggaaggcaggtcagagaccccactggacaaacagtggctggactctgcaccataacacacaatcaacaggggagtgagctggatccttatttctggtccctaagtgggtggtttgggcttactggggaggagctaaggccggagaggaggtactgaaggggagagtcctggacctttggcagcaaagggtgggacttctgcagtttctgtttccttgactggcagctcagcggggccctcccgcttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtaagcgagggtaaccttcccttcctgctgtctccagcatccctccttggccttttggggccaggcttcatcagcctttctcttcaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagaggtttgagggcaatgagtgtgggcagtggcctaaggcagaaacagggcaggcggcagcaaggtcaggactaggatgagactaggcagggtgacaaggtgggctgaccgggagtaggagcagttttagggtggcaggcggaaagggggcaagaaaaagcggagttaacccttactaagcatttaccctgggcttccaggcagccctggaagtcaagagaacactcagaaatggggagggagaagcagtggaaatccatatgggttgaggagtaggtaagatgctgcttctgcgggactgggaatgcgctgtttctcagtgacatggtctccgagaccaggagggatacacctaaggcagcctttccctcttgatgacttctacttgtccccccttctcaaagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggaggtgagaagcatcccctccccctacattgctgtctccctgacggcgcccagcccgaggagtgggcactcggctccggacactgtaactcttgctctctaccttgctcacggggcctcaggcttcagtgcttacctcgatgtctcatacctctgcagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaaggtgacctttgacctgtacccccaggtcagatcctggtcttccatcctactgtcttctctccccacctcaaccctgctctttcctcactttgtttaaacctccctgtacaactatctcacttctgagccttttataccctggaaacccatgatcccccgtctctttggtcactgtatccctgacactcccagacatttgacctcatttctgactctcccagactccttcatgtacgacacccctcaagaggtggccgaagctttcctgtcttccctgacagagaccatagaaggagtcgatgctgaggatgggcacggcccaggtttgaagacagagaagggaggcagggcagggaactgggggaaaatggagaagggacagaactgttaatgctggagcctgagccactctcctggcacccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggaatcctcctatccctgaactcgggggaatggaatctcgctgatcttccaggactagctccctgatcattccagcccctctgaacaacagggccccaggaaaatctccaggtcctattctgtcctccttcccttttacttgaagcagtttcttgactggtaattcctccatgaacctcagcccttgagcctcttactgagagcctccctgtcccagcaaagtcgctgaaatctcccaatcacagtattctattttcaatgccatggcgccttgttctcctcacccacaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaaggtcagaggttagggaatggtgggaggttcactttggggtcaggaggttcagggtggagggggtcatgagactaccttgagggcgacagggaggaccactttgtagtcaaaagttgaacagcaggatcgttgggcaatggaggttagtgggaacctgttgggggctggaagggccactttgtggtcaaagggaagtccgtgtaatgatgattaacttaaaaagttgaaagatgtgggatttcagttgcagattggtctctggggttaaaagatggcttggaagaccaggtgaggtgatggtctcttccctctccacagaccacaagttgaagtcagggactaacaccaagaaggccctccaggcagtgtacagcatgatgagctggccagatgacgtccctcctgaaggctggaaccgcacccgccatgtcatcatcctcatgactgatggtcagaagggacctctctcctgtcccagcctccccaccttctcagaccagcatgtggcccttaagtccacttgtaacactatacccatggttggggccctgaatgtgactcatagctggctgttcatctctcctgtgacccttcataaggaattcttcctaagccctgtgatcaactatctctaacccttcctcaacttgctcaccctgccatgtgtatccctgcctttagccagtttatcttccttatctcctaccctcatggtcctgtctcttctgcaggattgcacaacatgggcggggacccaattactgtcattgatgagatccgggacttgctatacattggcaaggatcgcaaaaacccaagggaggattatctgggtgagtaacctgcctaggacccagcaccccacttcctcagggcttggaccctcatccttcctttttatccctcagatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcggtagggagatacaagggaataaagaacacaactctcctcaggttcccctgaagtaattcattcttcctctacacctgaagctctagttgcctggaaagccttcttcattcctccttctctacctcagtgtcactattcttgtttcctggcactgttcacttaaccttagaatcacagagctctgagcacttcagagatctttctatagtcctacatttgacacgtggaaacagaagccaaaggaggtcaagggacagcaagttagcaacaagggtgggcttgaaaacagccaggcctctgacagcttgatcccaagttctttcccttttcagtccaccatagcagttttctcctaacacgaggaaacaaatacccgtggtctttccctttctccttttgggcctttgctccccatagactcctacccaaaaggctgctgccatttgggaatgaagtgttccgagttttcagcacattctccttctctgccagatgaaagccagtctctgagtctctgtggcatggtttgggaacacaggaagggtaccgattaccacaagcaaccatggcaggccaagatctcagtcattgtaagcacagaatcccagtagtggggacttgggggaggtgaggtcaaggtgaaatgggagtaggggaaggaaaaaatggccataagagatggtggtttgtgaaagttgagctttccctctctactgttgtgtccccagcgcccttcaaagggacacgagagctgtatgggggctgtggtgtctgagtactttgtgctgacagcagcacattgtttcactgtggatgacaaggaacactcaatcaaggtcagcgtaggtaaggatgcaactgaaggtcctgggctgcacctatgctctccaggcaacacctcccactttctacagatcctacactccacccatcctcaatgcagccccattccttgcaccccagaccagtcagggatgggggaagacgtgaagttaggaatgacacggggccagaggcaggaagctgcccacaaagaggtggtacctactctcctacttcaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggtgagagcgtccagatccctgaggaaaggctgggaaaggctggaggactggggtgaggagcaggcctggtttgctgttctccttgtcctttataggcccatttgtctcccctgcaccgagggaacaactcgagctttgaggcttcctccaactaccacttgccagcaacaaagtaagacatacttggcaagaggataaggatgagatcccaagagacaagtggggcatgagagggaggtgcaataggaagagatgatgcctggcccagaacctagctctagaagggcttaggggacatctactgagtgacaaaggcaatggggagatgacagtggtgggagcagctgaagtgacgcagtctattcgtccagaggaagagctgctccctgcacaggatatcaaagctctgtttgtgtctgaggaggagaaaaagctgactcggaaggaggtctacatcaagaatggggataaggtgagaaacgggcatcctaaggaggcactctaggccccaatccttcctaagccacttctgttcattacttctccatgcttcccacctcccctacagaaaggcagctgtgagagagatgctcaatatgccccaggctatgacaaagtcaaggacatctcagaggtggtcacccctcggttcctttgtactggaggagtgagtccctatgctgaccccaatacttgcagaggtgagagaatgctctttggttgtgctacaagtgcccaaggcccaacagtccttttctctacagcttctcctctccttgcaggtgattctggcggccccttgatagttcacaagagaagtcgtttcattcaagtgagtcctccctttcctatctggggagatgccaagtggtcagcatgggccccaaagcaggaaagctcaatgcatgtggctagtaattcgaggtaggcagagcctgcctcaccttaggaccgcatgtcttgcctgcgtgtgtcaagaacgaggctgagctgggtccctagtctgattcctttaggtcagctaagacacaagcaggaacagccatgcttccaggattaggaattctactgaatgatccatggcaccccactgcctctgcaggttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaacacctgtgttccagatccttttggggcaagggagtggggaacaggcactggccatgttgttacactgagatcaaacctgacagccgtttttaaaggtttaaccccaatcccaagtgctgaaaaaccagaggctgagggagatgtgtaagcttccacctcagtgttttactgagaccagcattggggcatatgaggcacaaggaatccagctctgttccctagaagccatccacaaggttttccttgtagacgtcatcactgtagacaatctgggtcctcttgtcccggtggcaacccttagggctgttctggacagctagggagggaggagaggaacagttaaggtctaaaggagatcatagaacagaccctgaggctgactcctgaccacctcactcctggccactggcccctggaagcccagtttccacgctgccctctggtggccaggatggcctgtcttccttagctcctttgtgccaacccatggccaagaaaagtataagtggacattttgatgaatgttttgttcttagaaaaatcccaaatgtcattgttgagacacgtgaatgatattaacccactacttacagtcagtatgtcagaagctaaaaactagaaaacctctgtagcccttttttgacatgctggtcaattctagttcctttcttttgcctgaagggccactgtagctgagcccttctttctgctcactcctttcccaggaaaatctactttcagggaaaatggattattcacactaagaaatgctactagctccaccagaactcattcagggtgtagctttggccctcaccattctctctcaagcctctagctgtttcttccccttcctctttcctccctccaccagacatgttactctcttcaccccatccaatggttccatccccaccacccttgagctacagagaatctctctcacccactcccatcctgtgatctctgtgcctcaacactgctggctactccctctttctcaaagtgtgtgtccttttgcttcagtggcccaggcccctgcggtgctgctcccagccctccgacccctcctcctgtctcctttgctaacgttaggctcaacgttagcctaacatgtcaggacagctggggacatgtgggg

SEQ ID NO: 3 - DNA, 9001 bp, Macaca mulatta
  source 1..9001: mol_type = genomic DNA
gatggaatcttgctctgtctcccacactggagtgcagtggcacgatcttggctcactgcgacttctgcctcccagatttaagtgattctcctacctcggcctcccaagtagctgggattataggtgcttgccaccacatgcagctaatttttgtatttttagtagagacaggattccgccatgttggccaggatggtcttgaactcctgacctcaagtgattcgcccacctcagcctcccaaactgctgggattacaggcgtgagccattgcacccagtcaggtctgcttattcttcccttctctctggttccacccctacggcagtggacaagctgtgccgaggtcgtctcccaagaaaaaaccatgttccccaacttgacagatgtcagggaggtggtgacagaccagtttctatgcagtgggacccaggaggatgagagtccctgcaagggtgagtccctcaccatgcctggattcccaaaggggaaggccacctgtgtctctgtggccagggtgcatgccagaacaccagtccactgccctgtatgacgctgtctcctgtcaccctttgctggcaggagaatctgggggagcagttttccttgagcggagattcaggttttttcaggtgagaaggtggaagcttgcaggacccaggggttacaggatgtcagccttgttggggggatgagggaggcctttgagggatctagggaggttggggcttacagctggagctgtggcagcctcccagccagttctctccttttctccaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctttagtcatggccactgagccctctgctgtcctgttagaatccgccccccctccatcttctacctctgaatgcccacccttagactctgtgacccatgctgtctcctagttgagtaaatctgggtctctaggatgccaggggcagcgcacacaagctgggaaatcctcagggctcctaccagcgggactgcctcgctgccccacctcccgctccttggcctgtccccaaattcctcccctggttgacttgactcatgctcatttcactttcatatggaatttcccagttatgaaattaataaaaatcagtggtttccacatctgtctgtgactctatctggaggccaggtagggctggcctggggggaaggggaggccagaatgactccaagagccacaggaaggcaggtcagagaccccactggacaaacagtggctggactctgtaccataacacacaagcaacaggggagtgagctggatccttatttctggtccctaagtgggtggtctgggcttgctggggaggagctgaggccagaaggaggtactgaaggggagagtcctggaccttgggcagcaaagggtgggacttctgcagtttctgcttccttgactggcagctcagcggggccctcccgcttggatgttccgggaaagtgatgagggtaggacaggcggggcaagctgcaggtgccagaacacagattgcataaaaggccgggagctggtggggggcaggggaagggaatgtgaccaggtctaggtctggagtttcagcttggacactgagctaagtagacaagcaaaacaagccaggacacgccatcctgccccaggcccagcttctctcctgccttctaacgccatggggagcagtctcagcccccagctctacctgatgcccttcatcttgggcctcttatctggaggtaagtgagggtaaccttcccttcctgctgtccccagcatccctccttggccttttggggccaggcttcatcagcctttctcttcaggtgtgaccaccactccattgtcttcggcccagcctcaaggatcctgctctctggagggggtagagatcaaaggtggctccttccgacttctccaagagggccaggcactggaatacgtgtgtccttctggcttctacccgtaccctgtgcagacacgtacctgcagatccacggggtcctggagcaccctgcagactcaagatcgaaaaactgtcaagaaggcagagtgcagaggtttgagggcaatgagtgtgggcagtggcctaagggagaaacagggcagatggcagcaaggtcaggactaggatgagactaggcagggtgacaaggtgggctgaccaggagtaggagcagttttagggttgtagagggaaaggaagggaaaaaaaaaggggagttaacctttagtaagcatttaccctgggcttccacgcagccctggaagtcaagagaacactcagcaatggggagggaggagcagcggaaacccctatgggttgaagggtaggtaagatgcagcttctgcaggactgggaatgctctgtttctcagtgacctggtctctgagaccaggagggaaacacctaaggcagcctttccctcttaatgacttctacttctcccctcttctcaaagcaatccgctgtccacgaccacaggacttcgagaacggggaataccggccccggtctccctactacaatgtgagtgatgagatctctttccactgctatgacggttacactctccggggctctgccaatcgcacctgccaagtgaatggccggtggagtgggcagacagcgatctgtgacaacggaggtgagaagcatcctctccccccacattgctgtctccctgacagcgcctagcctgaggagtgggcatttgcccccggacactgtaactcttgctctctaccttgccctcggggcctcaggcttcagcgcttacctccatgtctcatgcctctgcagcggggtactgctccaacccaggcatccccattggcacaaggaaggtgggcagccggtaccgccttgaagacagcgtcacctaccactgcagccgggggcttaccctgcgtggctcccagcggcgaacatgtcaggaaggtggctcttggagcgggacggagccttcctgccaaggtgacccttgacctgtacccccaggtcagatcctgatcttgcatcctactgtcttctctccccacctcaaccctgctctttcctcacttcttttaaaccttcctctagaactgtctcacttctgagccttttctaccctggaaacccacaatcccctgtctctttggtcactgtgtccctgacactcccagacatttgacctcatttctgactctcccagactccttcatgtacgacacccctcaagaggtggccgaagctttcctgtcttccctgacggagaccatagaaggagtcgatgccgaggatgggcacagcccaggtttgaaggcagagaggggaggcaaggcagggaactgggggaaaatggagaagggacaagataatcgttcatgctggagcctgagtcactctcctggcacccaggggaacaacagaagcggaggatcatcctagacccttcaggctccatgaacatctacctggtgctagatggatcagacagcattggggccggcaacttcacaggagccaaaaagtgtctagtcaacttaattgagaaggtggagtcctcctatccctgaacttgggggaatggaatcttgctgatcttccaggactagctccctgatcattccagcccctctgaaccgcagggccccaggaaagtctccaggtcctattctgtcctccttcccttgtacttgattcctccatgaacctgtgcttgagcctcttcctaagagcctccctgtcccagcaacgttgctgaagtctcccaatcacagtattctactttcaatgccatggcgccttgttctcctcacccacaggtggcaagttatggtgtgaagccaagatatgctctagtgacatatgccacataccccagaatttgggtcaaagtgtctgaccaagagagcagcaatgcagactgggtcacgaagaagctcagtgaaatcaattatgaaggtcagaggttagggaatggtgggaggttcactttggggtcaggaggttcaggagtgttgtgtggagggggtcatgagactaccttgagggcaacagggggaccactttgtagtcaaaggttgaacagcaggatcattgggcaatggaggttagtgggaacctgctgagggctggaagggccactttgtggtcaaagggaagtccatatgatgattaacttaaaaagttgaagatgtgagatttcagttgcagattggtctctggggttaaaagatggcttggaagaccaggtgaggcgatgctctcttccctccccacagaccacaagttgaagtcagggactaacaccaagagggccctccaggcagtgtacagcatgatgagttggccagaggacatccctcctgaaggctggaaccgcacccgccatgtcatcatcctcatgaccgatggtcagaagggacctctctcctgtcccagcctccccaccttctcagaccagcatgtggcccttaagtccacttgtaacactatacccatggttggggccctgaatgtgactcgtaactggctgttcatctctcctgtgacccttcataaagaattattcctaaagccctgtgatcaactacctctaacccttcctcaacttactcaccctgccacgtgtatcactgcctctagccaatttatcttatctcctaccctcatggtcccgtctcttctgcaggattgcacaacatgggcggggacccaattactgtcattgatgagatccgggacttgttatacatcggcaaggatcgtaaaaacccgagggaggattatctgggtgagtaacctgcctaggacccagcaccctacttcctcagggcttggaccgtcatccttcctttttctccctcagatgtctatgtgtttggggttggacctttggtggaccaagtgaacatcaatgctttggcttccaagaaagacaatgagcaacatgtgttcaaagtcaaggatatggaaaacctggaagacgttttcttccaaatgattggtaggcagacacaagggaatcaagaacgcaactctcctcagcttcccctgaaataattcattcttcctctacccctgaagctctagttgcctggaaagccttcttcattcctccttctctacctcagtatcactattcttgtttcctggcactgtttgcttcttaaccttagaatcacagagctctaggcacttcagagatctttctattgtcctacatttgacacatgtggaaacaaaggccaaaggaggtcaaggggcagcaagctagcaacagggctgggcttgaaaacagccaggcctctgatagcttgatcccaagttctttcccttttcactccaccacagcagttttctcctaacacgaggaaacaaatacctgtggcctttccctttctccttttgggcctctgccccccacagacttctacccaaaggctgctgccgtttgggaatgaagtgttccaagttttcagcacattctccttctctgccagatgaaagccagtctctgagtctctgtggcatggtttgggaacacagcaagggtaccgattaccacaagcaaccatggcaggccaagatctcagtcactgtaagcacagaatcccagtagtgaggacttgggggaggtgaggtcaaggtgaaatgggagtaggggaagggcaaaatggccgtaagagatggtggtttgtgaaagttgagttttccctttctactgttctgttcccagcgcccttcgaagggacatgagagctgtatgggggctgtggtgtctgagtactttgtgctgacagcagcacattgttttactgtggacgacaaggaacactcaatcaaggtcagcgtgggtaaggatgcaactgaaggtcccgggctgcacctacgccctccaggcaacacctcccactttctacagatcccacactccactcatctgcaatgcagccccatcccttgcaccccagaccagtcagggatggggaagacttgaagttaggaatgacatggggccagaggcaagaagctgcccacaaagaggtggtacctattctcctacttcaagggaagaagcgggacctggagatagaaaaagtcctatttcaccccgactacaacattagcgagaaaaaagaagcaggaattcctgaattttatgactatgacgttgccctgatcaagctcaagaataagttgaattatgacccgactatcaggtgagagcatccagatccctgaggaaaggctgggaaaggctggaggactggggtgaggagcaggcctagtttgctgttctttctccatcctttataggcccatttgtctcccctgcaccgagggaacaactcgagctttgaggcttcctccaactaccacttgccagcaacagagtaagacatactaggggggaggataaggatgagatcccgagacaagtgaggcatgagagggagatgcaataggaagagacgatgcctggcccagaacctagcactaggaagggcttaggggacatctgctgagtgacaaagtcaatagggagatgacagtggtgggagcagctgaagtgatgcagtctatttgtccagaggaagagctgctccctgcacaggatatcaaagctctgtttgtgtctgaggaggagaagaagctgactcggaaggaggtctacatcaagaatggggataaggtgagaaatgggcatcctaaggaggcactctaggccctaatccttcctaagccacctctgttcattacctttctccatgcttcccacctcccctacagaaaggcagctgtgagagagatgctcaatatgccccaggctatgacaaagtcaaggacatctcggaggtggtcacccctcggttcctttgtactggaggagtgagtccctatgctgaccccaatacttgcagaggtgagagaacgctctctggttgtgctccaagtgcccgagggccaagagtccttttccctacagcttctcctctccttgcaggtgattctggcggccccttgatagttcacaagagaagtcgtttcattcaagtgagtcctccctttcctatctggggagatgccaagtggtcagcatgggccccaaagcaggaaagcacaatgcatgtggctagtaattcgaggtgggcagagcctgcctcactttaggactgcatgtctggcctgtgtgtgtcaagaatgaggctgagctgggtccctagcctgattcctttaggtcagctaagacacaatcaggaacagtcatgcttccaggattaggaattctatgaatgatccatggcaccccactgcctctgcaggttggtgtcatcagctggggagtagtggatgtctgcaaaaaccagaagcggcaaaagcaggtacctgctcacgcccgagactttcacgtcaacctcttccaagtgctgccctggctgaaggagaaactccaagatgaggatttgggttttctctaaggggtttcctgctggacaggggcgcgggattgaattaaaacagctgcgacaacacttgtgttccagatccttttggggcaagggagtggggaacgggcactggccatgttgttacactgagatcaaacctgacacccatttttaaaggcttaaccccaatcccaagtgctgaaaaaccagaggctgagggagatatgtaagcttccacctcagtgttttactgagaccagcattggggcatttgaggcacaaggaatccagctctgttccctagaagccatccacaaggttttccttgtagacgtcatcactgtagacaatctgggtcctcttgtcccggtggcaacccttagggctgttctggacagctagggagggaggagaggaacagttaaggtctaaaggagatcatagatcagaccctgaggctgactcctgaccacctcagtcctggctgctggcccctggaaacccagtttccacgctgccctctggtggccaggatggcctgtcttccttagctcctttgtgccaacccatggccaaggagagtgtaagtggacattttgatgaatgttttgttcttagaaaaatcccaaatgtcattgttgagatatatgaatgatattaacccactacttatagtcagtatgtcagaagctaaaaactagaaaacctctgtagccctttattgacatgctggtcaactctagttcctttcttttgcctgaaaggccactgttgctctgagtccttctttctgctcactcctttcccaggaaaatctactttcaggtaaatgggttactcatactaaggaatgctactagctccaccagaactcatccagcatgtagctttggccctcaccattctctctcaagcctctagctgtttcttccccttcctcttttcctccctccaccagacatgttactctcttcaccccatccaaagattccatccccaccacccttgacctagagagaatctctcccacccacttctcatcctgtgatctctgtaccttgacactgctggctactccctctttctcaaagcatgtgtcctttcgcttcagtggcccaggcccctctggtgctgctcccagccctctgacccctcctcctgtctcctttgctaacgttaggctcaacgttagcctaacgtgtcaggagagctggagacacgtggggcgtaaggtggacagtcctgtttcctaacatagtccctgagtattcctcaagtctagtcctgggtcgttttttttctccgaaatcagtctccctcatgatcggggagccaccctgtgatgcagatgacttaatctatgttttcattccttacctcacacctgagttccagacccctaatttcaaatactt

SEQ ID NO: 4 - DNA, 4086 bp, Macaca mulatta
  source 1..4086: mol_type = genomic DNA
atagatatattagcatcagggagacagggcaaaggttccacccttcagctcagtccccagtccctgcttattatttccctaacagaagaccatcccccttgccactccctgggttttcttctctggcagcaatgaagcagctgctgagccagctctggttttcgggaagtcagatgaccttttccctcccgcggctctctgcctctcgctgtccctagggaggacaccatggacccactgatggttcttttttgcctgctgttcctgtacccaggtccggcagactcggctacctcctgccctcagaacgtgaatatctctggtggcaccttcaccctcagccatggctgggcccctgggagccttctcatctactcctgtccccagggcctgtacccatccccagcgtcacggctgtgcaagagcagcggacagtggcagaccccaagagccacccggtctctgactaaggcggtctgcaaacctggccactgccccaaccccggcatttcgctgggcgcggtgcggacaggctcccgctttggtcatggggacaaggtccgctatcgctgctcctcgaatcttgtgctcacggggtctgcggagcgggagtgccagggcaacggggtctggagtggaacggagcccatctgccgccagccctactcttatgacttccctgaggacgtggcccctgccctgggcacctccttctcccacatgcttggggccaccaatcccacccagaggacaaaggatcatgaaaatggaactgggactaacacctatgcagccctaaacagtgtctatctcatgatgaacaatcaaatgcaactccttggcatgaaaacgatggcctggcaggaaatccgacatgccatcatccttctgacagatggaaagtccaatatgggtggctctcccaaaacagctgttgaccaaatcagagagatcttgaatatcaaccagaagaggaatgactatctggacatctatgccatcggggtgggcaagctggatgtggactggagagaactgaatgagctggggtccaagaaggatggcgagaggcatgccttcattctgcaggacacaaaggctctgcaccaggtctttgaacatatgctggatgtctccaagctcacagacaccatctgcggggtggggaacatgtcagcaaacgcctctgaccaagagaggacaccctggcatgtcactattaagcccaagagccaagagacctgccggggagccctcatctccgaccaatgggtcctgacagcggctcactgcttccgcgatggcaacgaccactccctatggagggtcaatgtgggagaccccaaatcccagtggggcaaagaattccttattgagaaggcagtgatttccccaggatttgatgtctttgccaaaaagaaccagggaatcctggagttctatggtgatgacatcgccctgctgaagctggcccagaaagtaaagatgtccacccatgccaggcccatctgccttccctgcaccatggaggccaatctggctctgcggagacctcaaggcagcacctgtagggaccatgagaatgaactgctgaacaaacagagtgttcctgctcattttgtcgccttgaatgggagcaaactgaacattaaccttaagatgggagtggagtggacaagctgtgccgaggtcgtctcccaagaaaaaaccatgttccccaacttgacagatgtcagggaggtggtgacagaccagtttctatgcagtgggacccaggaggatgagagtccctgcaagggtgtgaccaccactccattgtcttcggcccagcctcaaggatcctgctctctggagggggtagagatcaaaggtggctccttccgacttctccaagagggccaggcactggaatacgtgtgtccttctggcttctacccgtaccctgtgcagacacgtacctgcagatccacggggtcctggagcaccctgcagactcaagatcgaaaaactgtcaagaaggcagagtgcagagcaatccgctgtccacgaccacaggacttcgagaacggggaataccggccccggtctccctactacaatgtgagtgatgagatctctttccactgctatgacggttacactctccggggctctgccaatcgcacctgccaagtgaatggccggtggagtgggcagacagcgatctgtgacaacggagcggggtactgctccaacccaggcatccccattggcacaaggaaggtgggcagccggtaccgccttgaagacagcgtcacctaccactgcagccgggggcttaccctgcgtggctcccagcggcgaacatgtcaggaaggtggctcttggagcgggacggagccttcctgccaagactccttcatgtacgacacccctcaagaggtggccgaagctttcctgtcttccctgacggagaccatagaaggagtcgatgccgaggatgggcacagcccaggggaacaacagaagcggaggatcatcctagacccttcaggctccatgaacatctacctggtgctagatggatcagacagcattggggccggcaacttcacaggagccaaaaagtgtctagtcaacttaattgagaaggtggcaagttatggtgtgaagccaagatatgctctagtgacatatgccacataccccagaatttgggtcaaagtgtctgaccaagagagcagcaatgcagactgggtcacgaagaagctcagtgaaatcaattatgaagaccacaagttgaagtcagggactaacaccaagagggccctccaggcagtgtacagcatgatgagttggccagaggacatccctcctgaaggctggaaccgcacccgccatgtcatcatcctcatgaccgatggattgcacaacatgggcggggacccaattactgtcattgatgagatccgggacttgttatacatcggcaaggatcgtaaaaacccgagggaggattatctggatgtctatgtgtttggggttggacctttggtggaccaagtgaacatcaatgctttggcttccaagaaagacaatgagcaacatgtgttcaaagtcaaggatatggaaaacctggaagacgttttcttccaaatgattgatgaaagccagtctctgagtctctgtggcatggtttgggaacacagcaagggtaccgattaccacaagcaaccatggcaggccaagatctcagtcactcgcccttcgaagggacatgagagctgtatgggggctgtggtgtctgagtactttgtgctgacagcagcacattgttttactgtggacgacaaggaacactcaatcaaggtcagcgtgggagggaagaagcgggacctggagatagaaaaagtcctatttcaccccgactacaacattagcgagaaaaaagaagcaggaattcctgaattttatgactatgacgttgccctgatcaagctcaagaataagttgaattatgacccgactatcaggcccatttgtctcccctgcaccgagggaacaactcgagctttgaggcttcctccaactaccacttgccagcaacagaaggaagagctgctccctgcacaggatatcaaagctctgtttgtgtctgaggaggagaagaagctgactcggaaggaggtctacatcaagaatggggataagaaaggcagctgtgagagagatgctcaatatgccccaggctatgacaaagtcaaggacatctcggaggtggtcacccctcggttcctttgtactggaggagtgagtccctatgctgaccccaatacttgcagaggtgattctggcggccccttgatagttcacaagagaagtcgtttcattcaagttggtgtcatcagctggggagtagtggatgtctgcaaaaaccagaagcggcaaaagcaggtacctgctcacgcccgagactttcacgtcaacctcttccaagtgctgccctggctgaaggagaaactccaagatgaggatttgggttttctctaaggggtttcctgctggacaggggcgcgggattgaattaaaacagctgcgacaacactt

SEQ ID NO: 5 - DNA, 2767 bp, Mus musculus
  source 1..2767: mol_type = genomic DNA
gctccatcacacagtccatggaaagactgatcttttaaattgggggtagtggaggtggtggtctgtgcttgttaggaggggtctgggggctaagagggagctttgaaagggaagttctggcccttggtcagtcaagggtggggctcacatagtttctgtttcctcagttggcagttcagctggggccctccttcatgaatgttccgggaagcagtggctgcgtgcgcagggtaggctggccaggctgcagatgccagagcagattgcataaaaggttaggggacagtgggaaaggggtgtagccagatccagcatttgggtttcagtttggacaggaggtcaaataggcacccagagtgacctggagagggctttgggccactggactctctggtgctttccatgacaatggagagcccccagctctgcctcgtcctcttggtcttaggcttctcctctggaggtgtgagcgcaactccagtgcttgaggcccggccccaagtctcctgctctctggagggagtagagatcaaaggcggctcctttcaacttctccaaggcggtcaggccctggagtacctatgtccctctggcttctacccataccccgtgcagactcgaacctgcagatccacaggctcctggagcgacctgcagacccgagaccaaaagattgtccagaaggcggaatgcagagcaatacgctgcccacgaccgcaggactttgaaaatggggaattctggccccggtcccccttctacaacctgagtgaccagatttcttttcaatgctatgatggttacgttctccggggctctgctaatcgcacctgccaagagaatggccggtgggatgggcaaacagcaatttgtgatgatggagctggatactgtcccaatcccggtattcctattgggacaaggaaggtgggtagccaataccgccttgaagacattgttacttaccactgcagccggggacttgtcctgcgtggctcccagaagcgaaagtgtcaagaaggtggctcatggagtgggacagagccttcctgccaagattccttcatgtatgacagccctcaagaagtggccgaagcattcctatcctccctgacagagaccatcgaaggagccgatgctgaggatgggcacagcccaggagaacagcagaagaggaagattgtcctagacccctcgggctccatgaatatctacctggtgctagatggatcagacagcatcggaagcagcaacttcacaggggctaagcggtgcctcaccaacttgattgagaaggtggcgagttacggggtgaggccacgatatggtctcctgacatatgctacagtccccaaagtgttggtcagagtgtctgatgagaggagtagcgatgccgactgggtcacagagaagctcaaccaaatcagttatgaagaccacaagctgaagtcagggaccaacaccaagagggctctccaggctgtgtatagcatgatgagctgggcaggggatgccccgcctgaaggctggaatagaacccgccatgtcatcatcattatgactgatggcttgcacaacatgggtggaaaccctgtcactgtcattcaggacatccgagccttgctggacatcggcagggatcccaaaaatcccagggaggattacctggatgtgtatgtgtttggggtcgggcctctggtggactccgtgaacatcaatgccttagcttccaaaaaggacaatgagcatcatgtgtttaaagtcaaggatatggaagacctggagaatgttttctaccaaatgattgatgaaaccaaatctctgagtctctgtggcatggtgtgggagcataaaaaaggcaacgattatcataagcaaccatggcaagccaagatctcagtcactcgccctctgaaaggacatgagacctgtatgggggccgtggtgtctgagtacttcgtgctgacagcagcgcactgcttcatggtggatgatcagaaacattccatcaaggtcagcgtggggggtcagaggcgggacctggagattgaagaggtcctgttccaccccaaatacaatattaatgggaaaaaggcagaagggatccctgagttctatgattatgatgtggccctagtcaagctcaagaacaagctcaagtatggccagactctcaggcccatctgtctcccctgcacggagggaaccacacgagccttgaggcttcctcagacagccacctgcaagcagcacaaggaacagttgctccctgtgaaggatgtcaaagctctgtttgtatctgagcaagggaagagcctgactcggaaggaggtgtacatcaagaatggggacaagaaagccagttgtgagagagatgctacaaaggcccaaggctatgagaaggtcaaagatgcctctgaggtggtcactccacggttcctctgcacaggaggggtggatccctatgctgaccccaacacatgcaaaggagattccgggggccctctcattgttcacaagagaagccgcttcattcaagttggtgtgattagctggggagtagtagatgtctgcagagaccagaggcggcaacagctggtaccctcttatgcccgggacttccacatcaacctcttccaggtgctgccctggctaaaggacaagctcaaagatgaggatttgggttttctataaagagcttcctgcagggagagtgtgaggacagattaaagcagttacaataacaaaaaaaaaaaaaaaaaaaaaa

SEQ ID NO: 6 - DNA, 20 bp, synthetic construct
  misc_feature 1..20: note = Synthetic Oligonucleotide
  source 1..20: mol_type = other DNA
gctgagctgccagtcaagga

SEQ ID NO: 7 - DNA, 20 bp, synthetic construct
  misc_feature 1..20: note = Synthetic Oligonucleotide
  source 1..20: mol_type = other DNA
ggccccgctgagctgccagt

SEQ ID NO: 8 - DNA, 20 bp, synthetic construct
  misc_feature 1..20: note = Synthetic Oligonucleotide
  source 1..20: mol_type = other DNA
cggaacatccaagcgggagg

SEQ ID NO: 9 - DNA, 20 bp, synthetic construct
  misc_feature 1..20: note = Synthetic Oligonucleotide
  source 1..20: mol_type = other DNA
ctttcccggaacatccaagc

SEQ ID NO: 10 - DNA, 20 bp, synthetic construct
  misc_feature 1..20: note = Synthetic Oligonucleotide
  source 1..20: mol_type = other DNA
atctgtgttctggcacctgc

SEQ ID NO: 11 - DNA, 20 bp, synthetic construct
  misc_feature 1..20: note = Synthetic Oligonucleotide
  source 1..20: mol_type = other DNA
gtcacattcccttcccctgc

SEQ ID NO: 12 - DNA, 20 bp, synthetic construct
  misc_feature 1..20: note = Synthetic Oligonucleotide
  source 1..20: mol_type = other DNA
gacctggtcacattcccttc

SEQ ID NO: 13 - DNA, 20 bp, synthetic construct
  misc_feature 1..20: note = Synthetic Oligonucleotide
  source 1..20: mol_type = other DNA
gacctagacctggtcacatt

SEQ ID NO: 14 - DNA, 20 bp, synthetic construct
  misc_feature 1..20: note = Synthetic Oligonucleotide
  source 1..20: mol_type = other DNA
actccagacctagacctggt

SEQ ID NO: 15 - DNA, 20 bp, synthetic construct
  misc_feature 1..20: note = Synthetic Oligonucleotide
  source 1..20: mol_type = other DNA
gctgaaactccagacctaga

SEQ ID NO: 16 - DNA, 20 bp, synthetic construct
  misc_feature 1..20: note = Synthetic Oligonucleotide
  source 1..20: mol_type = other DNA
gtccaagctgaaactccaga

SEQ ID NO: 17 - DNA, 20 bp, synthetic construct
  misc_feature 1..20: note = Synthetic Oligonucleotide
  source 1..20: mol_type = other DNA
ctcagtgtccaagctgaaac

SEQ ID NO: 18 - DNA, 20 bp, synthetic construct
  misc_feature 1..20: note = Synthetic Oligonucleotide
  source 1..20: mol_type = other DNA
aggagagaagctgggcctgg

SEQ ID NO: 19 - DNA, 20 bp, synthetic construct
  misc_feature 1..20: note = Synthetic Oligonucleotide
  source 1..20: mol_type = other DNA
gaaggcaggagagaagctgg

SEQ ID NO: 20 - DNA, 20 bp, synthetic construct
  misc_feature 1..20: note = Synthetic Oligonucleotide
  source 1..20: mol_type = other DNA
gtggtggtcacacctccaga

SEQ ID NO: 21 - DNA, 20 bp, synthetic construct
  misc_feature 1..20: note = Synthetic Oligonucleotide
  source 1..20: mol_type = other DNA
ccctccagagagcaggatcc

SEQ ID NO: 22 - DNA, 20 bp, synthetic construct
  misc_feature 1..20: note = Synthetic Oligonucleotide
  source 1..20: mol_type = other DNA
tctaccccctccagagagca

SEQ ID NO: 23 - DNA, 20 bp, synthetic construct
  misc_feature 1..20: note = Synthetic Oligonucleotide
  source 1..20: mol_type = other DNA
ttgatctctaccccctccag

SEQ ID NO: 24 - DNA, 20 bp, synthetic construct
  misc_feature 1..20: note = Synthetic Oligonucleotide
  source 1..20: mol_type = other DNA
tggagaagtcggaaggagcc

SEQ ID NO: 25 - DNA, 20 bp, synthetic construct
  misc_feature 1..20: note = Synthetic Oligonucleotide
  source 1..20: mol_type = other DNA
ccctcttggagaagtcggaa

SEQ ID NO: 26 - DNA, 20 bp, synthetic construct
  misc_feature 1..20: note = Synthetic Oligonucleotide
  source 1..20: mol_type = other DNA
gcctggccctcttggagaag

SEQ ID NO: 27 - DNA, 20 bp, synthetic construct
  misc_feature 1..20: note = Synthetic Oligonucleotide
  source 1..20: mol_type = other DNA
tccagtgcctggccctcttg

SEQ ID NO: 28 - DNA, 20 bp, synthetic construct
  misc_feature 1..20: note = Synthetic Oligonucleotide
  source 1..20: mol_type = other DNA
agaagccagaaggacacacg

SEQ ID NO: 29 - DNA, 20 bp, synthetic construct
  misc_feature 1..20: note = Synthetic Oligonucleotide
  source 1..20: mol_type = other DNA
acgggtagaagccagaagga

SEQ ID NO: 30 - DNA, 20 bp, synthetic construct
  misc_feature 1..20: note = Synthetic Oligonucleotide
  source 1..20: mol_type = other DNA
cgtgtctgcacagggtacgg

SEQ ID NO: 31 - DNA, 20 bp, synthetic construct
  misc_feature 1..20: note = Synthetic Oligonucleotide
  source 1..20: mol_type = other DNA
agggtgctccaggaccccgt

SEQ ID NO: 32 - DNA, 20 bp, synthetic construct
  misc_feature 1..20: note = Synthetic Oligonucleotide
  source 1..20: mol_type = other DNA
ttgctctgcactctgccttc

SEQ ID NO: 33 - DNA, 20 bp, synthetic construct
  misc_feature 1..20: note = Synthetic Oligonucleotide
  source 1..20: mol_type = other DNA
tattccccgttctcgaagtc

SEQ ID NO: 34 - DNA, 20 bp, synthetic construct
  misc_feature 1..20: note = Synthetic Oligonucleotide
  source 1..20: mol_type = other DNA
cattgtagtagggagaccgg

SEQ ID NO: 35 - DNA, 20 bp, synthetic construct
  misc_feature 1..20: note = Synthetic Oligonucleotide
  source 1..20: mol_type = other DNA
cactcacattgtagtaggga

SEQ ID NO: 36 - DNA, 20 bp, synthetic construct
  misc_feature 1..20: note = Synthetic Oligonucleotide
  source 1..20: mol_type = other DNA
tctcatcactcacattgtag

SEQ ID NO: 37 - DNA, 20 bp, synthetic construct
  misc_feature 1..20: note = Synthetic Oligonucleotide
  source 1..20: mol_type = other DNA
aagagatctcatcactcaca

SEQ ID NO: 38 - DNA, 20 bp, synthetic construct
  misc_feature 1..20: note = Synthetic Oligonucleotide
  source 1..20: mol_type = other DNA
agtggaaagagatctcatca

SEQ ID NO: 39 - DNA, 20 bp, synthetic construct
  misc_feature 1..20: note = Synthetic Oligonucleotide
  source 1..20: mol_type = other DNA
catagcagtggaaagagatc

SEQ ID NO: 40 - DNA, 20 bp, synthetic construct
  misc_feature 1..20: note = Synthetic Oligonucleotide
  source 1..20: mol_type = other DNA
aaccgtcatagcagtggaaa

SEQ ID NO: 41 - DNA, 20 bp, synthetic construct
  misc_feature 1..20: note = Synthetic Oligonucleotide
  source 1..20: mol_type = other DNA
gagtgtaaccgtcatagcag

SEQ ID NO: 42 - DNA, 20 bp, synthetic construct
  misc_feature 1..20: note = Synthetic Oligonucleotide
  source 1..20: mol_type = other DNA
cccggagagtgtaaccgtca

SEQ ID NO: 43 - DNA, 20 bp, synthetic construct
  misc_feature 1..20: note = Synthetic Oligonucleotide
  source 1..20: mol_type = other DNA
cagagccccggagagtgtaa

SEQ ID NO: 44 - DNA, 20 bp, synthetic construct
  misc_feature 1..20: note = Synthetic Oligonucleotide
  source 1..20: mol_type = other DNA
gattggcagagccccggaga

SEQ ID NO: 45 - DNA, 20 bp, synthetic construct
  misc_feature 1..20: note = Synthetic Oligonucleotide
  source 1..20: mol_type = other DNA
aggtgcgattggcagagccc

SEQ ID NO: 46 - DNA, 20 bp, synthetic construct
  misc_feature 1..20: note = Synthetic Oligonucleotide
  source 1..20: mol_type = other DNA
cttggcaggtgcgattggca

SEQ ID NO: 47 - DNA, 20 bp, synthetic construct
  misc_feature 1..20: note = Synthetic Oligonucleotide
  source 1..20: mol_type = other DNA
cattcacttggcaggtgcga

SEQ ID NO: 48 - DNA, 20 bp, synthetic construct
  misc_feature 1..20: note = Synthetic Oligonucleotide
  source 1..20: mol_type = other DNA
atcgctgtctgcccactcca

SEQ ID NO: 49 - DNA, 20 bp, synthetic construct
  misc_feature 1..20: note = Synthetic Oligonucleotide
  source 1..20: mol_type = other DNA
tcacagatcgctgtctgccc

SEQ ID NO: 50 - DNA, 20 bp, synthetic construct
  misc_feature 1..20: note = Synthetic Oligonucleotide
  source 1..20: mol_type = other DNA
ccgttgtcacagatcgctgt

SEQ ID NO: 51 - DNA, 20 bp, synthetic construct
  misc_feature 1..20: note = Synthetic Oligonucleotide
  source 1..20: mol_type = other DNA
cccgctccgttgtcacagat

SEQ ID NO: 52 - DNA, 20 bp, synthetic construct
  misc_feature 1..20: note = Synthetic Oligonucleotide
  source 1..20: mol_type = other DNA
cagtaccccgctccgttgtc

SEQ ID NO: 53 - DNA, 20 bp, synthetic construct
  misc_feature 1..20: note = Synthetic Oligonucleotide
  source 1..20: mol_type = other DNA
ttggagcagtaccccgctcc

SEQ ID NO: 54 - DNA, 20 bp, synthetic construct
  misc_feature 1..20: note = Synthetic Oligonucleotide
  source 1..20: mol_type = other DNA
accttccttgtgccaatggg

SEQ ID NO: 55 - DNA, 20 bp, synthetic construct
  misc_feature 1..20: note = Synthetic Oligonucleotide
  source 1..20: mol_type = other DNA
ctgcccaccttccttgtgcc

SEQ ID NO: 56 - DNA, 20 bp, synthetic construct
  misc_feature 1..20: note = Synthetic Oligonucleotide
  source 1..20: mol_type = other DNA
cgctgtcttcaaggcggtac

SEQ ID NO: 57 - DNA, 20 bp, synthetic construct
  misc_feature 1..20: note = Synthetic Oligonucleotide
  source 1..20: mol_type = other DNA
gctgcagtggtaggtgacgc

SEQ ID NO: 58 - DNA, 20 bp, synthetic construct
  misc_feature 1..20: note = Synthetic Oligonucleotide
  source 1..20: mol_type = other DNA
cccccggctgcagtggtagg

SEQ ID NO: 59 - DNA, 20 bp, synthetic construct
  misc_feature 1..20: note = Synthetic Oligonucleotide
  source 1..20: mol_type = other DNA
ggtaagcccccggctgcagt

SEQ ID NO: 60 - DNA, 20 bp, synthetic construct
  misc_feature 1..20: note = Synthetic Oligonucleotide
  source 1..20: mol_type = other DNA
acgcagggtaagcccccggc

SEQ ID NO: 61 - DNA, 20 bp, synthetic construct
  misc_feature 1..20: note = Synthetic Oligonucleotide
  source 1..20: mol_type = other DNA
ggagccacgcagggtaagcc

SEQ ID NO: 62 - DNA, 20 bp, synthetic construct
  misc_feature 1..20: note = Synthetic Oligonucleotide
  source 1..20: mol_type = other DNA
gccgctgggagccacgcagg

SEQ ID NO: 63 - DNA, 20 bp, synthetic construct
  misc_feature 1..20: note = Synthetic Oligonucleotide
  source 1..20: mol_type = other DNA
caagagccaccttcctgaca

SEQ ID NO: 64 - DNA, 20 bp, synthetic construct
  misc_feature 1..20: note = Synthetic Oligonucleotide
  source 1..20: mol_type = other DNA
ccgctccaagagccaccttc

SEQ ID NO: 65 - DNA, 20 bp, synthetic construct
  misc_feature 1..20: note = Synthetic Oligonucleotide
  source 1..20: mol_type = other DNA
tccgtcccgctccaagagcc

SEQ ID NO: 66 - DNA, 20 bp, synthetic construct
  misc_feature 1..20: note = Synthetic Oligonucleotide
  source 1..20: mol_type = other DNA
gaaggctccgtcccgctcca

SEQ ID NO: 67 - DNA, 20 bp, synthetic construct
  misc_feature 1..20: note = Synthetic Oligonucleotide
  source 1..20: mol_type = other DNA
tggcaggaaggctccgtccc

SEQ ID NO: 68 - DNA, 20 bp, synthetic construct
  misc_feature 1..20: note = Synthetic Oligonucleotide
  source 1..20: mol_type = other DNA
gagtcttggcaggaaggctc

SEQ ID NO: 69 - DNA, 20 bp, synthetic construct
  misc_feature 1..20: note = Synthetic Oligonucleotide
  source 1..20: mol_type = other DNA
atgaaggagtcttggcagga

SEQ ID NO: 70 - DNA, 20 bp, synthetic construct
  misc_feature 1..20: note = Synthetic Oligonucleotide
  source 1..20: mol_type = other DNA
cttcggccacctcttgaggg

SEQ ID NO: 71 - DNA, 20 bp, synthetic construct
  misc_feature 1..20: note = Synthetic Oligonucleotide
  source 1..20: mol_type = other DNA
ggaaagcttcggccacctct

SEQ ID NO: 72 - DNA, 20 bp, synthetic construct
  misc_feature 1..20: note = Synthetic Oligonucleotide
  source 1..20: mol_type = other DNA
aagacaggaaagcttcggcc

SEQ ID NO: 73 - DNA, 20 bp, synthetic construct
  misc_feature 1..20: note = Synthetic Oligonucleotide
  source 1..20: mol_type = other DNA
tcagggaagacaggaaagct

SEQ ID NO: 74 - DNA, 20 bp, synthetic construct
  misc_feature 1..20: note = Synthetic Oligonucleotide
  source 1..20: mol_type = other DNA
tcgactccttctatggtctc

SEQ ID NO: 75 - DNA, 20 bp, synthetic construct
  misc_feature 1..20: note = Synthetic Oligonucleotide
  source 1..20: mol_type = other DNA
cttctgttgttcccctgggc

SEQ ID NO: 76 - DNA, 20 bp, synthetic construct
  misc_feature 1..20: note = Synthetic Oligonucleotide
  source 1..20: mol_type = other DNA
ttcatggagcctgaagggtc

SEQ ID NO: 77 - DNA, 20 bp, synthetic construct
  misc_feature 1..20: note = Synthetic Oligonucleotide
  source 1..20: mol_type = other DNA
tagatgttcatggagcctga

SEQ ID NO: 78 - DNA, 20 bp, synthetic construct
  misc_feature 1..20: note = Synthetic Oligonucleotide
  source 1..20: mol_type = other DNA
accaggtagatgttcatgga

SEQ ID NO: 79 - DNA, 20 bp, synthetic construct
  misc_feature 1..20: note = Synthetic Oligonucleotide
  source 1..20: mol_type = other DNA
tctagcaccaggtagatgtt

SEQ ID NO: 80 - DNA, 20 bp, synthetic construct
  misc_feature 1..20: note = Synthetic Oligonucleotide
  source 1..20: mol_type = other DNA
gatccatctagcaccaggta

SEQ ID NO: 81 - DNA, 20 bp, synthetic construct
  misc_feature 1..20: note = Synthetic Oligonucleotide
  source 1..20: mol_type = other DNA
ctgtctgatccatctagcac

SEQ ID NO: 82 - DNA, 20 bp, synthetic construct
  misc_feature 1..20: note = Synthetic Oligonucleotide
  source 1..20: mol_type = other DNA
ccaatgctgtctgatccatc

SEQ ID NO: 83 - DNA, 20 bp, synthetic construct
  misc_feature 1..20: note = Synthetic Oligonucleotide
  source 1..20: mol_type = other DNA
tttggctcctgtgaagttgc

SEQ ID NO: 84 - DNA, 20 bp, synthetic construct
  misc_feature 1..20: note = Synthetic Oligonucleotide
  source 1..20: mol_type = other DNA
acactttttggctcctgtga

SEQ ID NO: 85 - DNA, 20 bp, synthetic construct
  misc_feature 1..20: note = Synthetic Oligonucleotide
  source 1..20: mol_type = other DNA
gactagacactttttggctc

SEQ ID NO: 86 - DNA, 20 bp, synthetic construct
  misc_feature 1..20: note = Synthetic Oligonucleotide
  source 1..20: mol_type = other DNA
taagttgactagacactttt

SEQ ID NO: 87 - DNA, 20 bp, synthetic construct
  misc_feature 1..20: note = Synthetic Oligonucleotide
  source 1..20: mol_type = other DNA
ctcaattaagttgactagac

SEQ ID NO: 88 - DNA, 20 bp, synthetic construct
  misc_feature 1..20: note = Synthetic Oligonucleotide
  source 1..20: mol_type = other DNA
caccttctcaattaagttga

SEQ ID NO: 89 - DNA, 20 bp, synthetic construct
  misc_feature 1..20: note = Synthetic Oligonucleotide
  source 1..20: mol_type = other DNA
acttgccaccttctcaatta

SEQ ID NO: 90 - DNA, 20 bp, synthetic construct
  misc_feature 1..20: note = Synthetic Oligonucleotide
  source 1..20: mol_type = other DNA
accataacttgccaccttct

SEQ ID NO: 91 - DNA, 20 bp, synthetic construct
  misc_feature 1..20: note = Synthetic Oligonucleotide
  source 1..20: mol_type = other DNA
cttcacaccataacttgcca

SEQ ID NO: 92 - DNA, 20 bp, synthetic construct
  misc_feature 1..20: note = Synthetic Oligonucleotide
  source 1..20: mol_type = other DNA
tcttggcttcacaccataac

SEQ ID NO: 93 - DNA, 20 bp, synthetic construct
  misc_feature 1..20: note = Synthetic Oligonucleotide
  source 1..20: mol_type = other DNA
atgtggcatatgtcactaga

SEQ ID NO: 94 - DNA, 20 bp, synthetic construct
  misc_feature 1..20: note = Synthetic Oligonucleotide
  source 1..20: mol_type = other DNA
cagacactttgacccaaatt

SEQ ID NO: 95 - DNA, 20 bp, synthetic construct
  misc_feature 1..20: note = Synthetic Oligonucleotide
  source 1..20: mol_type = other DNA
ggtcttcataattgatttca

SEQ ID NO: 96 - DNA, 20 bp, synthetic construct
  misc_feature 1..20: note = Synthetic Oligonucleotide
  source 1..20: mol_type = other DNA
acttgtggtcttcataattg

SEQ ID NO: 97 - DNA, 20 bp, synthetic construct
  misc_feature 1..20: note = Synthetic Oligonucleotide
  source 1..20: mol_type = other DNA
acttcaacttgtggtcttca

SEQ ID NO: 98 - DNA, 20 bp, synthetic construct
  misc_feature 1..20: note = Synthetic Oligonucleotide
  source 1..20: mol_type = other DNA
tccctgacttcaacttgtgg

SEQ ID NO: 99 - DNA, 20 bp, synthetic construct
  misc_feature 1..20: note = Synthetic Oligonucleotide
  source 1..20: mol_type = other DNA
tgttagtccctgacttcaac

SEQ ID NO: 100 - DNA, 20 bp, synthetic construct
  misc_feature 1..20: note = Synthetic Oligonucleotide
  source 1..20: mol_type = other DNA
tcttggtgttagtccctgac

SEQ ID NO: 101 - DNA, 20 bp, synthetic construct
  misc_feature 1..20: note = Synthetic Oligonucleotide
  source 1..20: mol_type = other DNA
tgtacactgcctggagggcc

SEQ ID NO: 102 - DNA, 20 bp, synthetic construct
  misc_feature 1..20: note = Synthetic Oligonucleotide
  source 1..20: mol_type = other DNA
tcatgctgtacactgcctgg

SEQ ID NO: 103 - DNA, 20 bp, synthetic construct
  misc_feature 1..20: note = Synthetic Oligonucleotide
  source 1..20: mol_type = other DNA
gttccagccttcaggaggga

SEQ ID NO: 104 - DNA, 20 bp, synthetic construct
  misc_feature 1..20: note = Synthetic Oligonucleotide
  source 1..20: mol_type = other DNA
ggtgcggttccagccttcag

SEQ ID NO: 105 - DNA, 20 bp, synthetic construct
  misc_feature 1..20: note = Synthetic Oligonucleotide
  source 1..20: mol_type = other DNA
atggcgggtgcggttccagc

SEQ ID NO: 106 - DNA, 20 bp, synthetic construct
  misc_feature 1..20: note = Synthetic Oligonucleotide
  source 1..20: mol_type = other DNA
gatgacatggcgggtgcggt

SEQ ID NO: 107 - DNA, 20 bp, synthetic construct
  misc_feature 1..20: note = Synthetic Oligonucleotide
  source 1..20: mol_type = other DNA
gaggatgatgacatggcggg

SEQ ID NO: 108 - DNA, 20 bp, synthetic construct
  misc_feature 1..20: note = Synthetic Oligonucleotide
  source 1..20: mol_type = other DNA
cccatgttgtgcaatccatc

SEQ ID NO: 109 - DNA, 20 bp, synthetic construct
  misc_feature 1..20: note = Synthetic Oligonucleotide
  source 1..20: mol_type = other DNA
tccccgcccatgttgtgcaa

SEQ ID NO: 110 - DNA, 20 bp, synthetic construct
  misc_feature 1..20: note = Synthetic Oligonucleotide
  source 1..20: mol_type = other DNA
attgggtccccgcccatgtt

SEQ ID NO: 111 - DNA, 20 bp, synthetic construct
  misc_feature 1..20: note = Synthetic Oligonucleotide
  source 1..20: mol_type = other DNA
acagtaattgggtccccgcc

SEQ ID NO: 112 - DNA, 20 bp, synthetic construct
  misc_feature 1..20: note = Synthetic Oligonucleotide
  source 1..20: mol_type = other DNA
tcaatgacagtaattgggtc

SEQ ID NO: 113 - DNA, 20 bp, synthetic construct
  misc_feature 1..20: note = Synthetic Oligonucleotide
  source 1..20: mol_type = other DNA
atctcatcaatgacagtaat

SEQ ID NO: 114 - DNA, 20 bp, synthetic construct
  misc_feature 1..20: note = Synthetic Oligonucleotide
  source 1..20: mol_type = other DNA
tcccggatctcatcaatgac

SEQ ID NO: 115 - DNA, 20 bp, synthetic construct
  misc_feature 1..20: note = Synthetic Oligonucleotide
  source 1..20: mol_type = other DNA
acatccagataatcctccct

SEQ ID NO: 116 - DNA, 20 bp, synthetic construct
  misc_feature 1..20: note = Synthetic Oligonucleotide
  source 1..20: mol_type = other DNA
acatagacatccagataatc

SEQ ID NO: 117 - DNA, 20 bp, synthetic construct
  misc_feature 1..20: note = Synthetic Oligonucleotide
  source 1..20: mol_type = other DNA
ccaaacacatagacatccag

SEQ ID NO: 118 - DNA, 20 bp, synthetic construct
  misc_feature 1..20: note = Synthetic Oligonucleotide
  source 1..20: mol_type = other DNA
agcattgatgttcacttggt

SEQ ID NO: 119 - DNA, 20 bp, synthetic construct
  misc_feature 1..20: note = Synthetic Oligonucleotide
  source 1..20: mol_type = other DNA
agccaaagcattgatgttca

SEQ ID NO: 120 - DNA, 20 bp, synthetic construct
  misc_feature 1..20: note = Synthetic Oligonucleotide
  source 1..20: mol_type = other DNA
cttggaagccaaagcattga

SEQ ID NO: 121 - DNA, 20 bp, synthetic construct
  misc_feature 1..20: note = Synthetic Oligonucleotide
  source 1..20: mol_type = other DNA
gtctttcttggaagccaaag

SEQ ID NO: 122 - DNA, 20 bp, synthetic construct
  misc_feature 1..20: note = Synthetic Oligonucleotide
  source 1..20: mol_type = other DNA
ctcattgtctttcttggaag

SEQ ID NO: 123 - DNA, 20 bp, synthetic construct
  misc_feature 1..20: note = Synthetic Oligonucleotide
  source 1..20: mol_type = other DNA
atgttgctcattgtctttct

SEQ ID NO: 124 - DNA, 20 bp, synthetic construct
  misc_feature 1..20: note = Synthetic Oligonucleotide
  source 1..20: mol_type = other DNA
gaacacatgttgctcattgt

SEQ ID NO: 125 - DNA, 20 bp, synthetic construct
  misc_feature 1..20: note = Synthetic Oligonucleotide
  source 1..20: mol_type = other DNA
gactttgaacacatgttgct

SEQ ID NO: 126 - DNA, 20 bp, synthetic construct
  misc_feature 1..20: note = Synthetic Oligonucleotide
  source 1..20: mol_type = other DNA
atccttgactttgaacacat

SEQ ID NO: 127 - DNA, 20 bp, synthetic construct
  misc_feature 1..20: note = Synthetic Oligonucleotide
  source 1..20: mol_type = other DNA
ttccatatccttgactttga

SEQ ID NO: 128 - DNA, 20 bp, synthetic construct
  misc_feature 1..20: note = Synthetic Oligonucleotide
  source 1..20: mol_type = other DNA
caggttttccatatccttga

SEQ ID NO: 129 - DNA, 20 bp, synthetic construct
  misc_feature 1..20: note = Synthetic Oligonucleotide
  source 1..20: mol_type = other DNA
ctcagagactggctttcatc

SEQ ID NO: 130 - DNA, 20 bp, synthetic construct
  misc_feature 1..20: note = Synthetic Oligonucleotide
  source 1..20: mol_type = other DNA
cagagactcagagactggct

SEQ ID NO: 131 - DNA, 20 bp, synthetic construct
  misc_feature 1..20: note = Synthetic Oligonucleotide
  source 1..20: mol_type = other DNA
atgccacagagactcagaga

SEQ ID NO: 132 - DNA, 20 bp, synthetic construct
  misc_feature 1..20: note = Synthetic Oligonucleotide
  source 1..20: mol_type = other DNA
caaaccatgccacagagact

SEQ ID NO: 133 - DNA, 20 bp, synthetic construct
  misc_feature 1..20: note = Synthetic Oligonucleotide
  source 1..20: mol_type = other DNA
tgttcccaaaccatgccaca

SEQ ID NO: 134 - DNA, 20 bp, synthetic construct
  misc_feature 1..20: note = Synthetic Oligonucleotide
  source 1..20: mol_type = other DNA
ttgtggtaatcggtaccctt

SEQ ID NO: 135 - DNA, 20 bp, synthetic construct
  misc_feature 1..20: note = Synthetic Oligonucleotide
  source 1..20: mol_type = other DNA
ggttgcttgtggtaatcggt

SEQ ID NO: 136 - DNA, 20 bp, synthetic construct
  misc_feature 1..20: note = Synthetic Oligonucleotide
  source 1..20: mol_type = other DNA
tgccatggttgcttgtggta

SEQ ID NO: 137 - DNA, 20 bp, synthetic construct
  misc_feature 1..20: note = Synthetic Oligonucleotide
  source 1..20: mol_type = other DNA
ttggcctgccatggttgctt

SEQ ID NO: 138 - DNA, 20 bp, synthetic construct
  misc_feature 1..20: note = Synthetic Oligonucleotide
  source 1..20: mol_type = other DNA
gagatcttggcctgccatgg

SEQ ID NO: 139 - DNA, 20 bp, synthetic construct
  misc_feature 1..20: note = Synthetic Oligonucleotide
  source 1..20: mol_type = other DNA
acagcccccatacagctctc

SEQ ID NO: 140 - DNA, 20 bp, synthetic construct
  misc_feature 1..20: note = Synthetic Oligonucleotide
  source 1..20: mol_type = other DNA
gacaccacagcccccataca

SEQ ID NO: 141 - DNA, 20 bp, synthetic construct
  misc_feature 1..20: note = Synthetic Oligonucleotide
  source 1..20: mol_type = other DNA
tactcagacaccacagcccc

SEQ ID NO: 142 - DNA, 20 bp, synthetic construct
  misc_feature 1..20: note = Synthetic Oligonucleotide
  source 1..20: mol_type = other DNA
acaaagtactcagacaccac

SEQ ID NO: 143 - DNA, 20 bp, synthetic construct
  misc_feature 1..20: note = Synthetic Oligonucleotide
  source 1..20: mol_type = other DNA
gtcagcacaaagtactcaga

SEQ ID NO: 144 - DNA, 20 bp, synthetic construct
  misc_feature 1..20: note = Synthetic Oligonucleotide
  source 1..20: mol_type = other DNA
ttgattgagtgttccttgtc

SEQ ID NO: 145 - DNA, 20 bp, synthetic construct
  misc_feature 1..20: note = Synthetic Oligonucleotide
  source 1..20: mol_type = other DNA
ctgaccttgattgagtgttc

SEQ ID NO: 146 - DNA, 20 bp, synthetic construct
  misc_feature 1..20: note = Synthetic Oligonucleotide
  source 1..20: mol_type = other DNA
tatctccaggtcccgcttct

SEQ ID NO: 147 - DNA, 20 bp, synthetic construct
  misc_feature 1..20: note = Synthetic Oligonucleotide
  source 1..20: mol_type = other DNA
gaattcctgcttcttttttc

SEQ ID NO: 148 - DNA, 20 bp, synthetic construct
  misc_feature 1..20: note = Synthetic Oligonucleotide
  source 1..20: mol_type = other DNA
attcaggaattcctgcttct

SEQ ID NO: 149 - DNA, 20 bp, synthetic construct
  misc_feature 1..20: note = Synthetic Oligonucleotide
  source 1..20: mol_type = other DNA
cataaaattcaggaattcct

SEQ ID NO: 150 - DNA, 20 bp, synthetic construct
  misc_feature 1..20: note = Synthetic Oligonucleotide
  source 1..20: mol_type = other DNA
catagtcataaaattcagga

SEQ ID NO: 151 - DNA, 20 bp, synthetic construct
  misc_feature 1..20: note = Synthetic Oligonucleotide
  source 1..20: mol_type = other DNA
tgagcttgatcagggcaacg

SEQ ID NO: 152 - DNA, 20 bp, synthetic construct
  misc_feature 1..20: note = Synthetic Oligonucleotide
  source 1..20: mol_type = other DNA
tattcttgagcttgatcagg

SEQ ID NO: 153 - DNA, 20 bp, synthetic construct
  misc_feature 1..20: note = Synthetic Oligonucleotide
  source 1..20: mol_type = other DNA
gacaaatgggcctgatagtc

SEQ ID NO: 154 - DNA, 20 bp, synthetic construct
  misc_feature 1..20: note = Synthetic Oligonucleotide
  source 1..20: mol_type = other DNA
gttgttccctcggtgcaggg

SEQ ID NO: 155 - DNA, 20 bp, synthetic construct
  misc_feature 1..20: note = Synthetic Oligonucleotide
  source 1..20: mol_type = other DNA
gctcgagttgttccctcggt

SEQ ID NO: 156 - DNA, 20 bp, synthetic construct
  misc_feature 1..20: note = Synthetic Oligonucleotide
  source 1..20: mol_type = other DNA
ctcaaagctcgagttgttcc

SEQ ID NO: 157 - DNA, 20 bp, synthetic construct
  misc_feature 1..20: note = Synthetic Oligonucleotide
  source 1..20: mol_type = other DNA
ggaagcctcaaagctcgagt

SEQ ID NO: 158 - DNA, 20 bp, synthetic construct
  misc_feature 1..20: note = Synthetic Oligonucleotide
  source 1..20: mol_type = other DNA
gttggaggaagcctcaaagc

SEQ ID NO: 159 - DNA, 20 bp, synthetic construct
  misc_feature 1..20: note = Synthetic Oligonucleotide
  source 1..20: mol_type = other DNA
gtggtagttggaggaagcct

SEQ ID NO: 160 - DNA, 20 bp, synthetic construct
  misc_feature 1..20: note = Synthetic Oligonucleotide
  source 1..20: mol_type = other DNA
tggcaagtggtagttggagg

SEQ ID NO: 161 - DNA, 20 bp, synthetic construct
  misc_feature 1..20: note = Synthetic Oligonucleotide
  source 1..20: mol_type = other DNA
tgttgctggcaagtggtagt

SEQ ID NO: 162 - DNA, 20 bp, synthetic construct
  misc_feature 1..20: note = Synthetic Oligonucleotide
  source 1..20: mol_type = other DNA
tccagctcactcccctgttg

SEQ ID NO: 163 - DNA, 20 bp, synthetic construct
  misc_feature 1..20: note = Synthetic Oligonucleotide
  source 1..20: mol_type = other DNA
taaggatccagctcactccc

SEQ ID NO: 164 - DNA, 20 bp, synthetic construct
  misc_feature 1..20: note = Synthetic Oligonucleotide
  source 1..20: mol_type = other DNA
cagaaataaggatccagctc

SEQ ID NO: 165 - DNA, 20 bp, synthetic construct
  misc_feature 1..20: note = Synthetic Oligonucleotide
  source 1..20: mol_type = other DNA
agggaccagaaataaggatc

SEQ ID NO: 166 - DNA, 20 bp, synthetic construct
  misc_feature 1..20: note = Synthetic Oligonucleotide
  source 1..20: mol_type = other DNA
ccacttagggaccagaaata

SEQ ID NO: 167 - DNA, 20 bp, synthetic construct
  misc_feature 1..20: note = Synthetic Oligonucleotide
  source 1..20: mol_type = other DNA
tccaggactctccccttcag

SEQ ID NO: 168 - DNA, 20 bp, synthetic construct
  misc_feature 1..20: note = Synthetic Oligonucleotide
  source 1..20: mol_type = other DNA
aagtcccaccctttgctgcc

SEQ ID NO: 169 - DNA, 20 bp, synthetic construct
  misc_feature 1..20: note = Synthetic Oligonucleotide
  source 1..20: mol_type = other DNA
ctgcagaagtcccacccttt

SEQ ID NO: 170 - DNA, 20 bp, synthetic construct
  misc_feature 1..20: note = Synthetic Oligonucleotide
  source 1..20: mol_type = other DNA
cagaaactgcagaagtccca

SEQ ID NO: 171 - DNA, 20 bp, synthetic construct
  misc_feature 1..20: note = Synthetic Oligonucleotide
  source 1..20: mol_type = other DNA
aacctctgcactctgccttc

SEQ ID NO: 172 - DNA, 20 bp, synthetic construct
  misc_feature 1..20: note = Synthetic Oligonucleotide
  source 1..20: mol_type = other DNA
ccctcaaacctctgcactct

SEQ ID NO: 173 - DNA, 20 bp, synthetic construct
  misc_feature 1..20: note = Synthetic Oligonucleotide
  source 1..20: mol_type = other DNA
tcattgccctcaaacctctg

SEQ ID NO: 174 - DNA, 20 bp, synthetic construct
  misc_feature 1..20: note = Synthetic Oligonucleotide
  source 1..20: mol_type = other DNA
ccacactcattgccctcaaa

SEQ ID NO: 175 - DNA, 20 bp, synthetic construct
  misc_feature 1..20: note = Synthetic Oligonucleotide
  source 1..20: mol_type = other DNA
cactgcccacactcattgcc

SEQ ID NO: 176 - DNA, 20 bp, synthetic construct
  misc_feature 1..20: note = Synthetic Oligonucleotide
  source 1..20: mol_type = other DNA
ttaggccactgcccacactc

SEQ ID NO: 177 - DNA, 20 bp, synthetic construct
  misc_feature 1..20: note = Synthetic Oligonucleotide
  source 1..20: mol_type = other DNA
ctagtcctgaccttgctgcc

SEQ ID NO: 178 - DNA, 20 bp, synthetic construct
  misc_feature 1..20: note = Synthetic Oligonucleotide
  source 1..20: mol_type = other DNA
ctcatcctagtcctgacctt

SEQ ID NO: 179 - DNA, 20 bp, synthetic construct
  misc_feature 1..20: note = Synthetic Oligonucleotide
  source 1..20: mol_type = other DNA
cctagtctcatcctagtcct

SEQ ID NO: 180 - DNA, 20 bp, synthetic construct
  misc_feature 1..20: note = Synthetic Oligonucleotide
  source 1..20: mol_type = other DNA
accctgcctagtctcatcct

SEQ ID NO: 181 - DNA, 20 bp, synthetic construct
  misc_feature 1..20: note = Synthetic Oligonucleotide
  source 1..20: mol_type = other DNA
cttgtcaccctgcctagtct

SEQ ID NO: 182 - DNA, 20 bp, synthetic construct
  misc_feature 1..20: note = Synthetic Oligonucleotide
  source 1..20: mol_type = other DNA
gcccaccttgtcaccctgcc

SEQ ID NO: 183 - DNA, 20 bp, synthetic construct
  misc_feature 1..20: note = Synthetic Oligonucleotide
  source 1..20: mol_type = other DNA
cctaaaactgctcctactcc

SEQ ID NO: 184 - DNA, 20 bp, synthetic construct
  misc_feature 1..20: note = Synthetic Oligonucleotide
  source 1..20: mol_type = other DNA
gagtcagaaatgaggtcaaa

SEQ ID NO: 185 - DNA, 20 bp, synthetic construct
  misc_feature 1..20: note = Synthetic Oligonucleotide
  source 1..20: mol_type = other DNA
ccctactcccatttcacctt

SEQ ID NO: 186 - DNA, 20 bp, synthetic construct
  misc_feature 1..20: note = Synthetic Oligonucleotide
  source 1..20: mol_type = other DNA
tgttgtgcaatcctgcagaa

SEQ ID NO: 187 - DNA, 20 bp, synthetic construct
  misc_feature 1..20: note = Synthetic Oligonucleotide
  source 1..20: mol_type = other DNA
aaaggctgatgaagcctggc

SEQ ID NO: 188 - DNA, 20 bp, synthetic construct
  misc_feature 1..20: note = Synthetic Oligonucleotide
  source 1..20: mol_type = other DNA
cctttgaccacaaagtggcc

SEQ ID NO: 189 - DNA, 20 bp, synthetic construct
  misc_feature 1..20: note = Synthetic Oligonucleotide
  source 1..20: mol_type = other DNA
aggtaccacctctttgtggg

SEQ ID NO: 190 - DNA, 20 bp, synthetic construct
  misc_feature 1..20: note = Synthetic Oligonucleotide
  source 1..20: mol_type = other DNA
tggtggtcacacctgaagag

SEQ ID NO: 191 - DNA, 20 bp, synthetic construct
  misc_feature 1..20: note = Synthetic Oligonucleotide
  source 1..20: mol_type = other DNA
gcagggagcagctcttcctt

SEQ ID NO: 192 - DNA, 20 bp, synthetic construct
  misc_feature 1..20: note = Synthetic Oligonucleotide
  source 1..20: mol_type = other DNA
tcctgtgcagggagcagctc

SEQ ID NO: 193 - DNA, 20 bp, synthetic construct
  misc_feature 1..20: note = Synthetic Oligonucleotide
  source 1..20: mol_type = other DNA
ttgatatcctgtgcagggag

SEQ ID NO: 194 - DNA, 20 bp, synthetic construct
  misc_feature 1..20: note = Synthetic Oligonucleotide
  source 1..20: mol_type = other DNA
agagctttgatatcctgtgc

SEQ ID NO: 195 - DNA, 20 bp, synthetic construct
  misc_feature 1..20: note = Synthetic Oligonucleotide
  source 1..20: mol_type = other DNA
acaaacagagctttgatatc

SEQ ID NO: 196 - DNA, 20 bp, synthetic construct
  misc_feature 1..20: note = Synthetic Oligonucleotide
  source 1..20: mol_type = other DNA
tcagacacaaacagagcttt

SEQ ID NO: 197 - DNA, 20 bp, synthetic construct
  misc_feature 1..20: note = Synthetic Oligonucleotide
  source 1..20: mol_type = other DNA
tcctcctcagacacaaacag

SEQ ID NO: 198 - DNA, 20 bp, synthetic construct
  misc_feature 1..20: note = Synthetic Oligonucleotide
  source 1..20: mol_type = other DNA
acctccttccgagtcagctt

SEQ ID NO: 199 - DNA, 20 bp, synthetic construct
  misc_feature 1..20: note = Synthetic Oligonucleotide
  source 1..20: mol_type = other DNA
atgtagacctccttccgagt

SEQ ID NO: 200 - DNA, 20 bp, synthetic construct
  misc_feature 1..20: note = Synthetic Oligonucleotide
  source 1..20: mol_type = other DNA
ttcttgatgtagacctcctt

SEQ ID NO: 201 - DNA, 20 bp, synthetic construct
  misc_feature 1..20: note = Synthetic Oligonucleotide
  source 1..20: mol_type = other DNA
tccccattcttgatgtagac

SEQ ID NO: 202 - DNA, 20 bp, synthetic construct
  misc_feature 1..20: note = Synthetic Oligonucleotide
  source 1..20: mol_type = other DNA
ttcttatccccattcttgat

SEQ ID NO: 203 - DNA, 20 bp, synthetic construct
  misc_feature 1..20: note = Synthetic Oligonucleotide
  source 1..20: mol_type = other DNA
ctgcctttcttatccccatt

SEQ ID NO: 204 - DNA, 20 bp, synthetic construct
  misc_feature 1..20: note = Synthetic Oligonucleotide
  source 1..20: mol_type = other DNA
tcacagctgcctttcttatc

SEQ ID NO: 205 - DNA, 20 bp, synthetic construct
  misc_feature 1..20: note = Synthetic Oligonucleotide
  source 1..20: mol_type = other DNA
tctctctcacagctgccttt

SEQ ID NO: 206 - DNA, 20 bp, synthetic construct
  misc_feature 1..20: note = Synthetic Oligonucleotide
  source 1..20: mol_type = other DNA
tgagcatctctctcacagct

SEQ ID NO: 207 - DNA, 20 bp, synthetic construct
  misc_feature 1..20: note = Synthetic Oligonucleotide
  source 1..20: mol_type = other DNA
gcatattgagcatctctctc

SEQ ID NO: 208 - DNA, 20 bp, synthetic construct
  misc_feature 1..20: note = Synthetic Oligonucleotide
  source 1..20: mol_type = other DNA
tgactttgtcatagcctggg

SEQ ID NO: 209 - DNA, 20 bp, synthetic construct
  misc_feature 1..20: note = Synthetic Oligonucleotide
  source 1..20: mol_type = other DNA
tgtccttgactttgtcatag

SEQ ID NO: 210 - DNA, 20 bp, synthetic construct
  misc_feature 1..20: note = Synthetic Oligonucleotide
  source 1..20: mol_type = other DNA
cagtacaaaggaaccgaggg

SEQ ID NO: 211 - DNA, 20 bp, synthetic construct
  misc_feature 1..20: note = Synthetic Oligonucleotide
  source 1..20: mol_type = other DNA
ctcctccagtacaaaggaac

SEQ ID NO: 212 - DNA, 20 bp, synthetic construct
  misc_feature 1..20: note = Synthetic Oligonucleotide
  source 1..20: mol_type = other DNA
gactcactcctccagtacaa

SEQ ID NO: 213 - DNA, 20 bp, synthetic construct
  misc_feature 1..20: note = Synthetic Oligonucleotide
  source 1..20: mol_type = other DNA
catagggactcactcctcca

SEQ ID NO: 214 - DNA, 20 bp, synthetic construct
  misc_feature 1..20: note = Synthetic Oligonucleotide
  source 1..20: mol_type = other DNA
ggtcagcatagggactcact

SEQ ID NO: 215 - DNA, 20 bp, synthetic construct
  misc_feature 1..20: note = Synthetic Oligonucleotide
  source 1..20: mol_type = other DNA
tcacctctgcaagtattggg

SEQ ID NO: 216 - DNA, 20 bp, synthetic construct
  misc_feature 1..20: note = Synthetic Oligonucleotide
  source 1..20: mol_type = other DNA
ccagaatcacctctgcaagt

SEQ ID NO: 217 - DNA, 20 bp, synthetic construct
  misc_feature 1..20: note = Synthetic Oligonucleotide
  source 1..20: mol_type = other DNA
gggccgccagaatcacctct

SEQ ID NO: 218 - DNA, 20 bp, synthetic construct
  misc_feature 1..20: note = Synthetic Oligonucleotide
  source 1..20: mol_type = other DNA
ctcttgtgaactatcaaggg

SEQ ID NO: 219 - DNA, 20 bp, synthetic construct
  misc_feature 1..20: note = Synthetic Oligonucleotide
  source 1..20: mol_type = other DNA
cgacttctcttgtgaactat

SEQ ID NO: 220 - DNA, 20 bp, synthetic construct
  misc_feature 1..20: note = Synthetic Oligonucleotide
  source 1..20: mol_type = other DNA
atgaaacgacttctcttgtg

SEQ ID NO: 221 - DNA, 20 bp, synthetic construct
  misc_feature 1..20: note = Synthetic Oligonucleotide
  source 1..20: mol_type = other DNA
acttgaatgaaacgacttct

SEQ ID NO: 222 - DNA, 20 bp, synthetic construct
  misc_feature 1..20: note = Synthetic Oligonucleotide
  source 1..20: mol_type = other DNA
acaccaacttgaatgaaacg

SEQ ID NO: 223 - DNA, 20 bp, synthetic construct
  misc_feature 1..20: note = Synthetic Oligonucleotide
  source 1..20: mol_type = other DNA
tccactactccccagctgat

SEQ ID NO: 224 - DNA, 20 bp, synthetic construct
  misc_feature 1..20: note = Synthetic Oligonucleotide
  source 1..20: mol_type = other DNA
cagacatccactactcccca

SEQ ID NO: 225 - DNA, 20 bp, synthetic construct
  misc_feature 1..20: note = Synthetic Oligonucleotide
  source 1..20: mol_type = other DNA
tttttgcagacatccactac

SEQ ID NO: 226 - DNA, 20 bp, synthetic construct
  misc_feature 1..20: note = Synthetic Oligonucleotide
  source 1..20: mol_type = other DNA
ttctggtttttgcagacatc

SEQ ID NO: 227 - DNA, 20 bp, synthetic construct
  misc_feature 1..20: note = Synthetic Oligonucleotide
  source 1..20: mol_type = other DNA
tgccgcttctggtttttgca

SEQ ID NO: 228 - DNA, 20 bp, synthetic construct
  misc_feature 1..20: note = Synthetic Oligonucleotide
  source 1..20: mol_type = other DNA
tgcttttgccgcttctggtt

SEQ ID NO: 229 - DNA, 20 bp, synthetic construct
  misc_feature 1..20: note = Synthetic Oligonucleotide
  source 1..20: mol_type = other DNA
ggtacctgcttttgccgctt

SEQ ID NO: 230 - DNA, 20 bp, synthetic construct
  misc_feature 1..20: note = Synthetic Oligonucleotide
  source 1..20: mol_type = other DNA
tgagcaggtacctgcttttg

SEQ ID NO: 231 - DNA, 20 bp, synthetic construct
  misc_feature 1..20: note = Synthetic Oligonucleotide
  source 1..20: mol_type = other DNA
ttcagccagggcagcacttg

SEQ ID NO: 232 - DNA, 20 bp, synthetic construct
  misc_feature 1..20: note = Synthetic Oligonucleotide
  source 1..20: mol_type = other DNA
ttctccttcagccagggcag

SEQ ID NO: 233 - DNA, 20 bp, synthetic construct
  misc_feature 1..20: note = Synthetic Oligonucleotide
  source 1..20: mol_type = other DNA
tggagtttctccttcagcca

SEQ ID NO: 234 - DNA, 20 bp, synthetic construct
  misc_feature 1..20: note = Synthetic Oligonucleotide
  source 1..20: mol_type = other DNA
tcatcttggagtttctcctt

SEQ ID NO: 235 - DNA, 20 bp, synthetic construct
  misc_feature 1..20: note = Synthetic Oligonucleotide
  source 1..20: mol_type = other DNA
aaatcctcatcttggagttt

SEQ ID NO: 236 - DNA, 20 bp, synthetic construct
  misc_feature 1..20: note = Synthetic Oligonucleotide
  source 1..20: mol_type = other DNA
aaacccaaatcctcatcttg

SEQ ID NO: 237 - DNA, 20 bp, synthetic construct
  misc_feature 1..20: note = Synthetic Oligonucleotide
  source 1..20: mol_type = other DNA
gtccagcaggaaacccctta

SEQ ID NO: 238 - DNA, 20 bp, synthetic construct
  misc_feature 1..20: note = Synthetic Oligonucleotide
  source 1..20: mol_type = other DNA
gcccctgtccagcaggaaac

SEQ ID NO: 239 - DNA, 20 bp, synthetic construct
  misc_feature 1..20: note = Synthetic Oligonucleotide
  source 1..20: mol_type = other DNA
agctgttttaattcaatccc

SEQ ID NO: 240 - DNA, 20 bp, synthetic construct
  misc_feature 1..20: note = Synthetic Oligonucleotide
  source 1..20: mol_type = other DNA
aacttgccacctgtgggtga

SEQ ID NO: 241 - DNA, 20 bp, synthetic construct
  misc_feature 1..20: note = Synthetic Oligonucleotide
  source 1..20: mol_type = other DNA
tcaccttatccccattcttg

SEQ ID NO: 242 - DNA, 20 bp, synthetic construct
  misc_feature 1..20: note = Synthetic Oligonucleotide
  source 1..20: mol_type = other DNA
tcaactttcacaaaccacca

SEQ ID NO: 243 - DNA, 20 bp, synthetic construct
  misc_feature 1..20: note = Synthetic Oligonucleotide
  source 1..20: mol_type = other DNA
ccgccagaatcacctgcaag

SEQ ID NO: 244 - DNA, 20 bp, synthetic construct
  misc_feature 1..20: note = Synthetic Oligonucleotide
  source 1..20: mol_type = other DNA
aggaggaatgaagaaggctt

SEQ ID NO: 245 - DNA, 20 bp, synthetic construct
  misc_feature 1..20: note = Synthetic Oligonucleotide
  source 1..20: mol_type = other DNA
gcctttcctcagggatctgg

SEQ ID NO: 246 - DNA, 20 bp, synthetic construct
  misc_feature 1..20: note = Synthetic Oligonucleotide
  source 1..20: mol_type = other DNA
aaatgtctgggagtgtcagg

SEQ ID NO: 247 - DNA, 20 bp, synthetic construct
  misc_feature 1..20: note = Synthetic Oligonucleotide
  source 1..20: mol_type = other DNA
gcctagagtgcctccttagg

SEQ ID NO: 248 - DNA, 20 bp, synthetic construct
  misc_feature 1..20: note = Synthetic Oligonucleotide
  source 1..20: mol_type = other DNA
ggcatctccccagataggaa

SEQ ID NO: 249 - DNA, 20 bp, synthetic construct
  misc_feature 1..20: note = Synthetic Oligonucleotide
  source 1..20: mol_type = other DNA
agggagctagtcctggaaga

SEQ ID NO: 250 - DNA, 20 bp, synthetic construct
  misc_feature 1..20: note = Synthetic Oligonucleotide
  source 1..20: mol_type = other DNA
acacctgaagagaaaggctg

SEQ ID NO: 251 - DNA, 20 bp, synthetic construct
  misc_feature 1..20: note = Synthetic Oligonucleotide
  source 1..20: mol_type = other DNA
ccctttgaccacaaagtggc

SEQ ID NO: 252 - DNA, 20 bp, synthetic construct
  misc_feature 1..20: note = Synthetic Oligonucleotide
  source 1..20: mol_type = other DNA
gccctcaaggtagtctcatg

SEQ ID NO: 253 - DNA, 20 bp, synthetic construct
  misc_feature 1..20: note = Synthetic Oligonucleotide
  source 1..20: mol_type = other DNA
aagggaaggaggacagaata

SEQ ID NO: 254 - DNA, 20 bp, synthetic construct
  misc_feature 1..20: note = Synthetic Oligonucleotide
  source 1..20: mol_type = other DNA
aaaggccaaggagggatgct

SEQ ID NO: 255 - DNA, 20 bp, synthetic construct
  misc_feature 1..20: note = Synthetic Oligonucleotide
  source 1..20: mol_type = other DNA
agaggtcccttctgaccatc

SEQ ID NO: 256 - DNA, 20 bp, synthetic construct
  misc_feature 1..20: note = Synthetic Oligonucleotide
  source 1..20: mol_type = other DNA
gctgggacaggagagaggtc

SEQ ID NO: 257 - DNA, 20 bp, synthetic construct
  misc_feature 1..20: note = Synthetic Oligonucleotide
  source 1..20: mol_type = other DNA
tcaaatgtctgggagtgtca

SEQ ID NO: 258 - DNA, 20 bp, synthetic construct
  misc_feature 1..20: note = Synthetic Oligonucleotide
  source 1..20: mol_type = other DNA
agaaggagaatgtgctgaaa

SEQ ID NO: 259 - DNA, 20 bp, synthetic construct
  misc_feature 1..20: note = Synthetic Oligonucleotide
  source 1..20: mol_type = other DNA
tgctgaccacttggcatctc

SEQ ID NO: 260 - DNA, 20 bp, synthetic construct
  misc_feature 1..20: note = Synthetic Oligonucleotide
  source 1..20: mol_type = other DNA
caactttcacaaaccaccat

SEQ ID NO: 261 - DNA, 20 bp, synthetic construct
  misc_feature 1..20: note = Synthetic Oligonucleotide
  source 1..20: mol_type = other DNA
agctctgtgattctaaggtt

SEQ ID NO: 262 - DNA, 20 bp, synthetic construct
  misc_feature 1..20: note = Synthetic Oligonucleotide
  source 1..20: mol_type = other DNA
ccacctgtgggtgaggagaa

SEQ ID NO: 263 - DNA, 20 bp, synthetic construct
  misc_feature 1..20: note = Synthetic Oligonucleotide
  source 1..20: mol_type = other DNA
gaggactcacttgaatgaaa

SEQ ID NO: 264 - DNA, 20 bp, synthetic construct
  misc_feature 1..20: note = Synthetic Oligonucleotide
  source 1..20: mol_type = other DNA
tggaatgatcagggagctag

SEQ ID NO: 265 - DNA, 20 bp, synthetic construct
  misc_feature 1..20: note = Synthetic Oligonucleotide
  source 1..20: mol_type = other DNA
gtcccttctccattttcccc

SEQ ID NO: 266 - DNA, 20 bp, synthetic construct
  misc_feature 1..20: note = Synthetic Oligonucleotide
  source 1..20: mol_type = other DNA
tcaactttttaagttaatca

SEQ ID NO: 267 - DNA, 20 bp, synthetic construct
  misc_feature 1..20: note = Synthetic Oligonucleotide
  source 1..20: mol_type = other DNA
gggtgaggagaacaaggcgc

SEQ ID NO: 268 - DNA, 20 bp, synthetic construct
  misc_feature 1..20: note = Synthetic Oligonucleotide
  source 1..20: mol_type = other DNA
cttccaagccatcttttaac

SEQ ID NO: 269 - DNA, 20 bp, synthetic construct
  misc_feature 1..20: note = Synthetic Oligonucleotide
  source 1..20: mol_type = other DNA
aggactcacttgaatgaaac

SEQ ID NO: 270 - DNA, 20 bp, synthetic construct
  misc_feature 1..20: note = Synthetic Oligonucleotide
  source 1..20: mol_type = other DNA
ttccaggcaactagagcttc

SEQ ID NO: 271 - DNA, 20 bp, synthetic construct
  misc_feature 1..20: note = Synthetic Oligonucleotide
  source 1..20: mol_type = other DNA
cagagtccagccactgtttg

SEQ ID NO: 272 - DNA, 20 bp, synthetic construct
  misc_feature 1..20: note = Synthetic Oligonucleotide
  source 1..20: mol_type = other DNA
ccaacctgcagaggcagtgg

SEQ ID NO: 273 - DNA, 20 bp, synthetic construct
  misc_feature 1..20: note = Synthetic Oligonucleotide
  source 1..20: mol_type = other DNA
tgcaaggagaggagaagctg

SEQ ID NO: 274 - DNA, 20 bp, synthetic construct
  misc_feature 1..20: note = Synthetic Oligonucleotide
  source 1..20: mol_type = other DNA
ctaggcaggttactcaccca

SEQ ID NO: 275 - DNA, 20 bp, synthetic construct
  misc_feature 1..20: note = Synthetic Oligonucleotide
  source 1..20: mol_type = other DNA
caccataacttgccacctgt

SEQ ID NO: 276 - DNA, 20 bp, synthetic construct
  misc_feature 1..20: note = Synthetic Oligonucleotide
  source 1..20: mol_type = other DNA
taggtaccacctctttgtgg

SEQ ID NO: 277 - DNA, 20 bp, synthetic construct
  misc_feature 1..20: note = Synthetic Oligonucleotide
  source 1..20: mol_type = other DNA
cttgacctcacctcccccaa

SEQ ID NO: 278 - DNA, 20 bp, synthetic construct
  misc_feature 1..20: note = Synthetic Oligonucleotide
  source 1..20: mol_type = other DNA
ccacctctttgtgggcagct

SEQ ID NO: 279 - DNA, 20 bp, synthetic construct
  misc_feature 1..20: note = Synthetic Oligonucleotide
  source 1..20: mol_type = other DNA
ttcacaaaccaccatctctt

SEQ ID NO: 280 - DNA, 20 bp, synthetic construct
  misc_feature 1..20: note = Synthetic Oligonucleotide
  source 1..20: mol_type = other DNA
ttctcacctccgttgtcaca

SEQ ID NO: 281 - DNA, 20 bp, synthetic construct
  misc_feature 1..20: note = Synthetic Oligonucleotide
  source 1..20: mol_type = other DNA
gaaagtgggaggtgttgcct

SEQ ID NO: 282 - DNA, 20 bp, synthetic construct
  misc_feature 1..20: note = Synthetic Oligonucleotide
  source 1..20: mol_type = other DNA
acagcaggaagggaaggtta

SEQ ID NO: 283 - DNA, 20 bp, synthetic construct
  misc_feature 1..20: note = Synthetic Oligonucleotide
  source 1..20: mol_type = other DNA
catgctgaccacttggcatc

SEQ ID NO: 284 - DNA, 20 bp, synthetic construct
  misc_feature 1..20: note = Synthetic Oligonucleotide
  source 1..20: mol_type = other DNA
ggtcaccttggcaggaaggc

SEQ ID NO: 285 - DNA, 20 bp, synthetic construct
  misc_feature 1..20: note = Synthetic Oligonucleotide
  source 1..20: mol_type = other DNA
gtatagtgttacaagtggac

SEQ ID NO: 286 - DNA, 20 bp, synthetic construct
  misc_feature 1..20: note = Synthetic Oligonucleotide
  source 1..20: mol_type = other DNA
ggacttccctttgaccacaa

SEQ ID NO: 287 - DNA, 20 bp, synthetic construct
  misc_feature 1..20: note = Synthetic Oligonucleotide
  source 1..20: mol_type = other DNA
tcaccttgacctcacctccc

SEQ ID NO: 288 - DNA, 20 bp, synthetic construct
  misc_feature 1..20: note = Synthetic Oligonucleotide
  source 1..20: mol_type = other DNA
tagagtgcctccttaggatg

SEQ ID NO: 289 - DNA, 20 bp, synthetic construct
  misc_feature 1..20: note = Synthetic Oligonucleotide
  source 1..20: mol_type = other DNA
tgacttcaacttgtggtctg

SEQ ID NO: 290 - DNA, 20 bp, synthetic construct
  misc_feature 1..20: note = Synthetic Oligonucleotide
  source 1..20: mol_type = other DNA
cagagaaggagaatgtgctg

SEQ ID NO: 291 - DNA, 20 bp, synthetic construct
  misc_feature 1..20: note = Synthetic Oligonucleotide
  source 1..20: mol_type = other DNA
agggagcagctcttcctctg

SEQ ID NO: 292 - DNA, 20 bp, synthetic construct
  misc_feature 1..20: note = Synthetic Oligonucleotide
  source 1..20: mol_type = other DNA
tgttcccctgggtgccagga

SEQ ID NO: 293 - DNA, 20 bp, synthetic construct
  misc_feature 1..20: note = Synthetic Oligonucleotide
  source 1..20: mol_type = other DNA
ggcctggctgttttcaagcc

SEQ ID NO: 294 - DNA, 20 bp, synthetic construct
  misc_feature 1..20: note = Synthetic Oligonucleotide
  source 1..20: mol_type = other DNA
gactggctttcatctggcag

SEQ ID NO: 295 - DNA, 20 bp, synthetic construct
  misc_feature 1..20: note = Synthetic Oligonucleotide
  source 1..20: mol_type = other DNA
gaaggctttccaggcaacta

SEQ ID NO: 296 - DNA, 20 bp, synthetic construct
  misc_feature 1..20: note = Synthetic Oligonucleotide
  source 1..20: mol_type = other DNA
tcacttgaatgaaacgactt

SEQ ID NO: 297 - DNA, 20 bp, synthetic construct
  misc_feature 1..20: note = Synthetic Oligonucleotide
  source 1..20: mol_type = other DNA
ggccccaaaaggccaaggag

SEQ ID NO: 298 - DNA, 20 bp, synthetic construct
  misc_feature 1..20: note = Synthetic Oligonucleotide
  source 1..20: mol_type = other DNA
aatcacctgcaaggagagga

SEQ ID NO: 299 - DNA, 20 bp, synthetic construct
  misc_feature 1..20: note = Synthetic Oligonucleotide
  source 1..20: mol_type = other DNA
gaccttcagttgcatcctta

SEQ ID NO: 300 - DNA, 20 bp, synthetic construct
  misc_feature 1..20: note = Synthetic Oligonucleotide
  source 1..20: mol_type = other DNA
tgatgaagcctggccccaaa

SEQ ID NO: 301 - DNA, 20 bp, synthetic construct
  misc_feature 1..20: note = Synthetic Oligonucleotide
  source 1..20: mol_type = other DNA
tagaaagtgggaggtgttgc

SEQ ID NO: 302 - DNA, 20 bp, synthetic construct
  misc_feature 1..20: note = Synthetic Oligonucleotide
  source 1..20: mol_type = other DNA
cccatccctgactggtctgg

SEQ ID NO: 303 - DNA, 20 bp, synthetic construct
  misc_feature 1..20: note = Synthetic Oligonucleotide
  source 1..20: mol_type = other DNA
ccatgggtatagtgttacaa

SEQ ID NO: 304 - DNA, 20 bp, synthetic construct
  misc_feature 1..20: note = Synthetic Oligonucleotide
  source 1..20: mol_type = other DNA
gtgttctcttgacttccagg

SEQ ID NO: 305 - DNA, 20 bp, synthetic construct
  misc_feature 1..20: note = Synthetic Oligonucleotide
  source 1..20: mol_type = other DNA
ggcctgctcctcaccccagt

SEQ ID NO: 306 - DNA, 20 bp, synthetic construct
  misc_feature 1..20: note = Synthetic Oligonucleotide
  source 1..20: mol_type = other DNA
gaggcctggctgttttcaag

SEQ ID NO: 307 - DNA, 20 bp, synthetic construct
  misc_feature 1..20: note = Synthetic Oligonucleotide
  source 1..20: mol_type = other DNA
gactctccccttcagtacct

SEQ ID NO: 308 - DNA, 20 bp, synthetic construct
  misc_feature 1..20: note = Synthetic Oligonucleotide
  source 1..20: mol_type = other DNA
catgggtatagtgttacaag

SEQ ID NO: 309 - DNA, 20 bp, synthetic construct
  misc_feature 1..20: note = Synthetic Oligonucleotide
  source 1..20: mol_type = other DNA
gaaggagaatgtgctgaaaa

SEQ ID NO: 310 - DNA, 20 bp, synthetic construct
  misc_feature 1..20: note = Synthetic Oligonucleotide
  source 1..20: mol_type = other DNA
tcacctggtcttccaagcca

SEQ ID NO: 311 - DNA, 20 bp, synthetic construct
  misc_feature 1..20: note = Synthetic Oligonucleotide
  source 1..20: mol_type = other DNA
ctccccagataggaaaggga

SEQ ID NO: 312 - DNA, 20 bp, synthetic construct
  misc_feature 1..20: note = Synthetic Oligonucleotide
  source 1..20: mol_type = other DNA
ggactcacttgaatgaaacg

SEQ ID NO: 313 - DNA, 20 bp, synthetic construct
  misc_feature 1..20: note = Synthetic Oligonucleotide
  source 1..20: mol_type = other DNA
ggccgccagaatcacctgca

SEQ ID NO: 314 - DNA, 20 bp, synthetic construct
  misc_feature 1..20: note = Synthetic Oligonucleotide
  source 1..20: mol_type = other DNA
ctcacttgaatgaaacgact

SEQ ID NO: 315 - DNA, 20 bp, synthetic construct
  misc_feature 1..20: note = Synthetic Oligonucleotide
  source 1..20: mol_type = other DNA
ctttcccagcctttcctcag

SEQ ID NO: 316 - DNA, 20 bp, synthetic construct
  misc_feature 1..20: note = Synthetic Oligonucleotide
  source 1..20: mol_type = other DNA
agaaagtgggaggtgttgcc

SEQ ID NO: 317 - DNA, 20 bp, synthetic construct
  misc_feature 1..20: note = Synthetic Oligonucleotide
  source 1..20: mol_type = other DNA
gtcgcagctgttttaattca

SEQ ID NO: 318 - DNA, 20 bp, synthetic construct
  misc_feature 1..20: note = Synthetic Oligonucleotide
  source 1..20: mol_type = other DNA
ccaggactctccccttcagt

SEQ ID NO: 319 - DNA, 20 bp, synthetic construct
  misc_feature 1..20: note = Synthetic Oligonucleotide
  source 1..20: mol_type = other DNA
agggaaggaggacagaatag

SEQ ID NO: 320 - DNA, 20 bp, synthetic construct
  misc_feature 1..20: note = Synthetic Oligonucleotide
  source 1..20: mol_type = other DNA
gaaatgaggtcaaatgtctg

SEQ ID NO: 321 - DNA, 20 bp, synthetic construct
  misc_feature 1..20: note = Synthetic Oligonucleotide
  source 1..20: mol_type = other DNA
ggagagtcagaaatgaggtc

SEQ ID NO: 322 - DNA, 20 bp, synthetic construct
  misc_feature 1..20: note = Synthetic Oligonucleotide
  source 1..20: mol_type = other DNA
gtagaaagtgggaggtgttg

SEQ ID NO: 323 - DNA, 20 bp, synthetic construct
  misc_feature 1..20: note = Synthetic Oligonucleotide
  source 1..20: mol_type = other DNA
tagaaagatctctgaagtgc

SEQ ID NO: 324 - DNA, 20 bp, synthetic construct
  misc_feature 1..20: note = Synthetic Oligonucleotide
  source 1..20: mol_type = other DNA
ctgctcctcaccccagtcct

SEQ ID NO: 325 - DNA, 20 bp, synthetic construct
  misc_feature 1..20: note = Synthetic Oligonucleotide
  source 1..20: mol_type = other DNA
ctactgggattctgtgctta

SEQ ID NO: 326 - DNA, 20 bp, synthetic construct
  misc_feature 1..20: note = Synthetic Oligonucleotide
  source 1..20: mol_type = other DNA
cccaaaaggccaaggaggga

SEQ ID NO: 327 - DNA, 20 bp, synthetic construct
  misc_feature 1..20: note = Synthetic Oligonucleotide
  source 1..20: mol_type = other DNA
tgaccacttggcatctcccc

SEQ ID NO: 328 - DNA, 20 bp, synthetic construct
  misc_feature 1..20: note = Synthetic Oligonucleotide
  source 1..20: mol_type = other DNA
cctgcaaggagaggagaagc

SEQ ID NO: 329 - DNA, 20 bp, synthetic construct
  misc_feature 1..20: note = Synthetic Oligonucleotide
  source 1..20: mol_type = other DNA
ctctcacctctgcaagtatt

SEQ ID NO: 330 - DNA, 20 bp, synthetic construct
  misc_feature 1..20: note = Synthetic Oligonucleotide
  source 1..20: mol_type = other DNA
ccccaaaaggccaaggaggg

SEQ ID NO: 331 - DNA, 20 bp, synthetic construct
  misc_feature 1..20: note = Synthetic Oligonucleotide
  source 1..20: mol_type = other DNA
gtcttccaagccatctttta

SEQ ID NO: 332 - DNA, 20 bp, synthetic construct
  misc_feature 1..20: note = Synthetic Oligonucleotide
  source 1..20: mol_type = other DNA
gttacaagtggacttaaggg

SEQ ID NO: 333 - DNA, 20 bp, synthetic construct
  misc_feature 1..20: note = Synthetic Oligonucleotide
  source 1..20: mol_type = other DNA
cccatgttgtgcaatcctgc

SEQ ID NO: 334 - DNA, 20 bp, synthetic construct
  misc_feature 1..20: note = Synthetic Oligonucleotide
  source 1..20: mol_type = other DNA
gaggtgggaagcatggagaa

SEQ ID NO: 335 - DNA, 20 bp, synthetic construct
  misc_feature 1..20: note = Synthetic Oligonucleotide
  source 1..20: mol_type = other DNA
tgctcccaccactgtcatct

SEQ ID NO: 336 - DNA, 20 bp, synthetic construct
  misc_feature 1..20: note = Synthetic Oligonucleotide
  source 1..20: mol_type = other DNA
aggcaggttactcacccaga

SEQ ID NO: 337 - DNA, 20 bp, synthetic construct
  misc_feature 1..20: note = Synthetic Oligonucleotide
  source 1..20: mol_type = other DNA
tactgggattctgtgcttac

SEQ ID NO: 338 - DNA, 20 bp, synthetic construct
  misc_feature 1..20: note = Synthetic Oligonucleotide
  source 1..20: mol_type = other DNA
gcctttcccagcctttcctc

SEQ ID NO: 339 - DNA, 20 bp, synthetic construct
  misc_feature 1..20: note = Synthetic Oligonucleotide
  source 1..20: mol_type = other DNA
gtgcaatcctgcagaagaga

SEQ ID NO: 340 - DNA, 20 bp, synthetic construct
  misc_feature 1..20: note = Synthetic Oligonucleotide
  source 1..20: mol_type = other DNA
acaggagagaggtcccttct

SEQ ID NO: 341 - DNA, 20 bp, synthetic construct
  misc_feature 1..20: note = Synthetic Oligonucleotide
  source 1..20: mol_type = other DNA
cccaaaaggagaaagggaaa

SEQ ID NO: 342 - DNA, 20 bp, synthetic construct
  misc_feature 1..20: note = Synthetic Oligonucleotide
  source 1..20: mol_type = other DNA
aagcccagggtaaatgctta

SEQ ID NO: 343 - DNA, 20 bp, synthetic construct
  misc_feature 1..20: note = Synthetic Oligonucleotide
  source 1..20: mol_type = other DNA
gatgaagcctggccccaaaa

SEQ ID NO: 344 - DNA, 20 bp, synthetic construct
  misc_feature 1..20: note = Synthetic Oligonucleotide
  source 1..20: mol_type = other DNA
tggcagagaaggagaatgtg

SEQ ID NO: 345 - DNA, 20 bp, synthetic construct
  misc_feature 1..20: note = Synthetic Oligonucleotide
  source 1..20: mol_type = other DNA
ttcccagcctttcctcaggg

SEQ ID NO: 346 - DNA, 20 bp, synthetic construct
  misc_feature 1..20: note = Synthetic Oligonucleotide
  source 1..20: mol_type = other DNA
ggcagagaaggagaatgtgc

SEQ ID NO: 347 - DNA, 20 bp, synthetic construct
  misc_feature 1..20: note = Synthetic Oligonucleotide
  source 1..20: mol_type = other DNA
acagtgccaggaaacaagaa

SEQ ID NO: 348 - DNA, 20 bp, synthetic construct
  misc_feature 1..20: note = Synthetic Oligonucleotide
  source 1..20: mol_type = other DNA
taggcaggttactcacccag

SEQ ID NO: 349 - DNA, 20 bp, synthetic construct
  misc_feature 1..20: note = Synthetic Oligonucleotide
  source 1..20: mol_type = other DNA
ttctcttgacttccagggct

SEQ ID NO: 350 - DNA, 20 bp, synthetic construct
  misc_feature 1..20: note = Synthetic Oligonucleotide
  source 1..20: mol_type = other DNA
cctgctcctcaccccagtcc

SEQ ID NO: 351 - DNA, 20 bp, synthetic construct
  misc_feature 1..20: note = Synthetic Oligonucleotide
  source 1..20: mol_type = other DNA
tcccactaacctccattgcc

SEQ ID NO: 352 - DNA, 20 bp, synthetic construct
  misc_feature 1..20: note = Synthetic Oligonucleotide
  source 1..20: mol_type = other DNA
ttccctttgaccacaaagtg

SEQ ID NO: 353 - DNA, 20 bp, synthetic construct
  misc_feature 1..20: note = Synthetic Oligonucleotide
  source 1..20: mol_type = other DNA
ctgggtcctaggcaggttac

SEQ ID NO: 354 - DNA, 20 bp, synthetic construct
  misc_feature 1..20: note = Synthetic Oligonucleotide
  source 1..20: mol_type = other DNA
tccaggcaactagagcttca

SEQ ID NO: 355 - DNA, 20 bp, synthetic construct
  misc_feature 1..20: note = Synthetic Oligonucleotide
  source 1..20: mol_type = other DNA
gcccatgttgtgcaatcctg

SEQ ID NO: 356 - DNA, 20 bp, synthetic construct
  misc_feature 1..20: note = Synthetic Oligonucleotide
  source 1..20: mol_type = other DNA
ggttcccactaacctccatt

SEQ ID NO: 357 - DNA, 20 bp, synthetic construct
  misc_feature 1..20: note = Synthetic Oligonucleotide
  source 1..20: mol_type = other DNA
aggtagagagcaagagttac

SEQ ID NO: 358 - DNA, 20 bp, synthetic construct
  misc_feature 1..20: note = Synthetic Oligonucleotide
  source 1..20: mol_type = other DNA
ccactaacctccattgccca

SEQ ID NO: 359 - DNA, 20 bp, synthetic construct
  misc_feature 1..20: note = Synthetic Oligonucleotide
  source 1..20: mol_type = other DNA
tcacaaaccaccatctctta

SEQ ID NO: 360 - DNA, 20 bp, synthetic construct
  misc_feature 1..20: note = Synthetic Oligonucleotide
  source 1..20: mol_type = other DNA
tactcacccagataatcctc

SEQ ID NO: 361 - DNA, 20 bp, synthetic construct
  misc_feature 1..20: note = Synthetic Oligonucleotide
  source 1..20: mol_type = other DNA
tgctcctcaccccagtcctc

SEQ ID NO: 362 - DNA, 20 bp, synthetic construct
  misc_feature 1..20: note = Synthetic Oligonucleotide
  source 1..20: mol_type = other DNA
tctcacagctgcctttctgt

SEQ ID NO: 363 - DNA, 20 bp, synthetic construct
  misc_feature 1..20: note = Synthetic Oligonucleotide
  source 1..20: mol_type = other DNA
gaaagggaggactcacttga

SEQ ID NO: 364 - DNA, 20 bp, synthetic construct
  misc_feature 1..20: note = Synthetic Oligonucleotide
  source 1..20: mol_type = other DNA
ccatcttttaaccccagaga

SEQ ID NO: 365 - DNA, 20 bp, synthetic construct
  misc_feature 1..20: note = Synthetic Oligonucleotide
  source 1..20: mol_type = other DNA
tcctcaccccagtcctccag

SEQ ID NO: 366 - DNA, 20 bp, synthetic construct
  misc_feature 1..20: note = Synthetic Oligonucleotide
  source 1..20: mol_type = other DNA
ctggcagagaaggagaatgt

SEQ ID NO: 367 - DNA, 20 bp, synthetic construct
  misc_feature 1..20: note = Synthetic Oligonucleotide
  source 1..20: mol_type = other DNA
tctccccagataggaaaggg

SEQ ID NO: 368 - DNA, 20 bp, synthetic construct
  misc_feature 1..20: note = Synthetic Oligonucleotide
  source 1..20: mol_type = other DNA
acttcagctgctcccaccac

SEQ ID NO: 369 - DNA, 20 bp, synthetic construct
  misc_feature 1..20: note = Synthetic Oligonucleotide
  source 1..20: mol_type = other DNA
gacagcaggaagggaaggtt

SEQ ID NO: 370 - DNA, 20 bp, synthetic construct
  misc_feature 1..20: note = Synthetic Oligonucleotide
  source 1..20: mol_type = other DNA
ggagacaaatgggcctataa

SEQ ID NO: 371 - DNA, 20 bp, synthetic construct
  misc_feature 1..20: note = Synthetic Oligonucleotide
  source 1..20: mol_type = other DNA
ctgctcccaccactgtcatc

SEQ ID NO: 372 - DNA, 20 bp, synthetic construct
  misc_feature 1..20: note = Synthetic Oligonucleotide
  source 1..20: mol_type = other DNA
aggaatgaagaaggctttcc

SEQ ID NO: 373 - DNA, 20 bp, synthetic construct
  misc_feature 1..20: note = Synthetic Oligonucleotide
  source 1..20: mol_type = other DNA
gggatctcatccttatcctc

SEQ ID NO: 374 - DNA, 20 bp, synthetic construct
  misc_feature 1..20: note = Synthetic Oligonucleotide
  source 1..20: mol_type = other DNA
gtgctgggtcctaggcaggt

SEQ ID NO: 375 - DNA, 20 bp, synthetic construct
  misc_feature 1..20: note = Synthetic Oligonucleotide
  source 1..20: mol_type = other DNA
caaaaggccaaggagggatg

SEQ ID NO: 376 - DNA, 20 bp, synthetic construct
  misc_feature 1..20: note = Synthetic Oligonucleotide
  source 1..20: mol_type = other DNA
ccatgctgaccacttggcat

SEQ ID NO: 377 - DNA, 20 bp, synthetic construct
  misc_feature 1..20: note = Synthetic Oligonucleotide
  source 1..20: mol_type = other DNA
ggaggctgggacaggagaga

SEQ ID NO: 378 - DNA, 20 bp, synthetic construct
  misc_feature 1..20: note = Synthetic Oligonucleotide
  source 1..20: mol_type = other DNA
ggagcagctcttcctctgga

SEQ ID NO: 379 - DNA, 20 bp, synthetic construct
  misc_feature 1..20: note = Synthetic Oligonucleotide
  source 1..20: mol_type = other DNA
tctcacctccgttgtcacag

SEQ ID NO: 380 - DNA, 20 bp, synthetic construct
  misc_feature 1..20: note = Synthetic Oligonucleotide
  source 1..20: mol_type = other DNA
cagtcctccagcctttccca

SEQ ID NO: 381 - DNA, 20 bp, synthetic construct
  misc_feature 1..20: note = Synthetic Oligonucleotide
  source 1..20: mol_type = other DNA
agtcctccagcctttcccag

SEQ ID NO: 382 - DNA, 20 bp, synthetic construct
  misc_feature 1..20: note = Synthetic Oligonucleotide
  source 1..20: mol_type = other DNA
tgaaggagtctgggagagtc

SEQ ID NO: 383 - DNA, 20 bp, synthetic construct
  misc_feature 1..20: note = Synthetic Oligonucleotide
  source 1..20: mol_type = other DNA
cagaatcacctgcaaggaga

SEQ ID NO: 384 - DNA, 20 bp, synthetic construct
  misc_feature 1..20: note = Synthetic Oligonucleotide
  source 1..20: mol_type = other DNA
taggaaagggaggactcact

SEQ ID NO: 385 - DNA, 20 bp, synthetic construct
  misc_feature 1..20: note = Synthetic Oligonucleotide
  source 1..20: mol_type = other DNA
accttggcaggaaggctccg

SEQ ID NO: 386 - DNA, 20 bp, synthetic construct
  misc_feature 1..20: note = Synthetic Oligonucleotide
  source 1..20: mol_type = other DNA
gagacaaatgggcctataaa

SEQ ID NO: 387 - DNA, 20 bp, synthetic construct
  misc_feature 1..20: note = Synthetic Oligonucleotide
  source 1..20: mol_type = other DNA
ctgaagagaaaggctgatga

SEQ ID NO: 388 - DNA, 20 bp, synthetic construct
  misc_feature 1..20: note = Synthetic Oligonucleotide
  source 1..20: mol_type = other DNA
aatgatcagggagctagtcc

SEQ ID NO: 389 - DNA, 20 bp, synthetic construct
  misc_feature 1..20: note = Synthetic Oligonucleotide
  source 1..20: mol_type = other DNA
cttagctgacctaaaggaat

SEQ ID NO: 390 - DNA, 20 bp, synthetic construct
  misc_feature 1..20: note = Synthetic Oligonucleotide
  source 1..20: mol_type = other DNA
tgggtatagtgttacaagtg

SEQ ID NO: 391 - DNA, 20 bp, synthetic construct
  misc_feature 1..20: note = Synthetic Oligonucleotide
  source 1..20: mol_type = other DNA
tgaagagaaaggctgatgaa

SEQ ID NO: 392 - DNA, 20 bp, synthetic construct
  misc_feature 1..20: note = Synthetic Oligonucleotide
  source 1..20: mol_type = other DNA
gtgttacaagtggacttaag

SEQ ID NO: 393 - DNA, 20 bp, synthetic construct
  misc_feature 1..20: note = Synthetic Oligonucleotide
  source 1..20: mol_type = other DNA
acctgtgggtgaggagaaca

SEQ ID NO: 394 - DNA, 20 bp, synthetic construct
  misc_feature 1..20: note = Synthetic Oligonucleotide
  source 1..20: mol_type = other DNA
tcacccagataatcctccct

SEQ ID NO: 395 - DNA, 20 bp, synthetic construct
  misc_feature 1..20: note = Synthetic Oligonucleotide
  source 1..20: mol_type = other DNA
tgttgtcgcagctgttttaa

SEQ ID NO: 396 - DNA, 20 bp, synthetic construct
  misc_feature 1..20: note = Synthetic Oligonucleotide
  source 1..20: mol_type = other DNA
tggtcacattcccttcccct

SEQ ID NO: 397 - DNA, 20 bp, synthetic construct
  misc_feature 1..20: note = Synthetic Oligonucleotide
  source 1..20: mol_type = other DNA
cctggtcacattcccttccc

SEQ ID NO: 398 - DNA, 20 bp, synthetic construct
  misc_feature 1..20: note = Synthetic Oligonucleotide
  source 1..20: mol_type = other DNA
tagacctggtcacattccct

SEQ ID NO: 399 - DNA, 20 bp, synthetic construct
  misc_feature 1..20: note = Synthetic Oligonucleotide
  source 1..20: mol_type = other DNA
cctagacctggtcacattcc

SEQ ID NO: 400 - DNA, 20 bp, synthetic construct
  misc_feature 1..20: note = Synthetic Oligonucleotide
  source 1..20: mol_type = other DNA
ccttccgagtcagctttttc

SEQ ID NO: 401 - DNA, 20 bp, synthetic construct
  misc_feature 1..20: note = Synthetic Oligonucleotide
  source 1..20: mol_type = other DNA
ctccttccgagtcagctttt

SEQ ID NO: 402 - DNA, 20 bp, synthetic construct
  misc_feature 1..20: note = Synthetic Oligonucleotide
  source 1..20: mol_type = other DNA
agacctccttccgagtcagc

SEQ ID NO: 403 - DNA, 20 bp, synthetic construct
  misc_feature 1..20: note = Synthetic Oligonucleotide
  source 1..20: mol_type = other DNA
gtagacctccttccgagtca

SEQ ID NO: 404 - DNA, 20 bp, synthetic construct
  misc_feature 1..20: note = Synthetic Oligonucleotide
  source 1..20: mol_type = other DNA
tttgccgcttctggtttttg

SEQ ID NO: 405 - DNA, 20 bp, synthetic construct
  misc_feature 1..20: note = Synthetic Oligonucleotide
  source 1..20: mol_type = other DNA
cttttgccgcttctggtttt

SEQ ID NO: 406 - DNA, 20 bp, synthetic construct
  misc_feature 1..20: note = Synthetic Oligonucleotide
  source 1..20: mol_type = other DNA
cctgcttttgccgcttctgg

SEQ ID NO: 407 - DNA, 20 bp, synthetic construct
  misc_feature 1..20: note = Synthetic Oligonucleotide
  source 1..20: mol_type = other DNA
tacctgcttttgccgcttct

SEQ ID NO: 408 - DNA, 20 bp, synthetic construct
  misc_feature 1..20: note = Synthetic Oligonucleotide
  source 1..20: mol_type = other DNA
agaaaacccaaatcctcatc

SEQ ID NO: 409 - DNA, 20 bp, synthetic construct
  misc_feature 1..20: note = Synthetic Oligonucleotide
  source 1..20: mol_type = other DNA
tagaaaacccaaatcctcat

SEQ ID NO: 410 - DNA, 20 bp, synthetic construct
  misc_feature 1..20: note = Synthetic Oligonucleotide
  source 1..20: mol_type = other DNA
atagaaaacccaaatcctca

SEQ ID NO: 411 - DNA, 20 bp, synthetic construct
  misc_feature 1..20: note = Synthetic Oligonucleotide
  source 1..20: mol_type = other DNA
tatagaaaacccaaatcctc

SEQ ID NO: 412 - DNA, 20 bp, synthetic construct
  misc_feature 1..20: note = Synthetic Oligonucleotide
  source 1..20: mol_type = other DNA
ttatagaaaacccaaatcct

SEQ ID NO: 413 - DNA, 20 bp, synthetic construct
  misc_feature 1..20: note = Synthetic Oligonucleotide
  source 1..20: mol_type = other DNA
cttatagaaaacccaaatcc

SEQ ID NO: 414 - DNA, 20 bp, synthetic construct
  misc_feature 1..20: note = Synthetic Oligonucleotide
  source 1..20: mol_type = other DNA
ccttatagaaaacccaaatc

SEQ ID NO: 415 - DNA, 20 bp, synthetic construct
  misc_feature 1..20: note = Synthetic Oligonucleotide
  source 1..20: mol_type = other DNA
cccttatagaaaacccaaat

SEQ ID NO: 416 - DNA, 20 bp, synthetic construct
  misc_feature 1..20: note = Synthetic Oligonucleotide
  source 1..20: mol_type = other DNA
ccccttatagaaaacccaaa

SEQ ID NO: 417 - DNA, 20 bp, synthetic construct
  misc_feature 1..20: note = Synthetic Oligonucleotide
  source 1..20: mol_type = other DNA
accccttatagaaaacccaa

SEQ ID NO: 418 - DNA, 20 bp, synthetic construct
  misc_feature 1..20: note = Synthetic Oligonucleotide
  source 1..20: mol_type = other DNA
aaccccttatagaaaaccca

SEQ ID NO: 419 - DNA, 20 bp, synthetic construct
  misc_feature 1..20: note = Synthetic Oligonucleotide
  source 1..20: mol_type = other DNA
aaaccccttatagaaaaccc

SEQ ID NO: 420 - DNA, 20 bp, synthetic construct
  misc_feature 1..20: note = Synthetic Oligonucleotide
  source 1..20: mol_type = other DNA
gaaaccccttatagaaaacc

SEQ ID NO: 421 - DNA, 20 bp, synthetic construct
  misc_feature 1..20: note = Synthetic Oligonucleotide
  source 1..20: mol_type = other DNA
ggaaaccccttatagaaaac

SEQ ID NO: 422 - DNA, 20 bp, synthetic construct
  misc_feature 1..20: note = Synthetic Oligonucleotide
  source 1..20: mol_type = other DNA
aggaaaccccttatagaaaa

SEQ ID NO: 423 - DNA, 20 bp, synthetic construct
  misc_feature 1..20: note = Synthetic Oligonucleotide
  source 1..20: mol_type = other DNA
caggaaaccccttatagaaa

SEQ ID NO: 424 - DNA, 20 bp, synthetic construct
  misc_feature 1..20: note = Synthetic Oligonucleotide
  source 1..20: mol_type = other DNA
gcaggaaaccccttatagaa

SEQ ID NO: 425 - DNA, 20 bp, synthetic construct
  misc_feature 1..20: note = Synthetic Oligonucleotide
  source 1..20: mol_type = other DNA
agcaggaaaccccttataga

SEQ ID NO: 426 - DNA, 20 bp, synthetic construct
  misc_feature 1..20: note = Synthetic Oligonucleotide
  source 1..20: mol_type = other DNA
cagcaggaaaccccttatag

SEQ ID NO: 427 - DNA, 20 bp, synthetic construct
  misc_feature 1..20: note = Synthetic Oligonucleotide
  source 1..20: mol_type = other DNA
ccagcaggaaaccccttata

SEQ ID NO: 428 - DNA, 20 bp, synthetic construct
  misc_feature 1..20: note = Synthetic Oligonucleotide
  source 1..20: mol_type = other DNA
tccagcaggaaaccccttat

SEQ ID NO: 429 - DNA, 20 bp, synthetic construct
  misc_feature 1..20: note = Synthetic Oligonucleotide
  source 1..20: mol_type = other DNA
tgtccagcaggaaacccctt

SEQ ID NO: 430 - DNA, 20 bp, synthetic construct
  misc_feature 1..20: note = Synthetic Oligonucleotide
  source 1..20: mol_type = other DNA
ctgtccagcaggaaacccct

SEQ ID NO: 431 - DNA, 20 bp, synthetic construct
  misc_feature 1..20: note = Synthetic Oligonucleotide
  source 1..20: mol_type = other DNA
cctgtccagcaggaaacccc

SEQ ID NO: 432 - DNA, 20 bp, synthetic construct
  misc_feature 1..20: note = Synthetic Oligonucleotide
  source 1..20: mol_type = other DNA
ccctgtccagcaggaaaccc

SEQ ID NO: 433 - DNA, 20 bp, synthetic construct
  misc_feature 1..20: note = Synthetic Oligonucleotide
  source 1..20: mol_type = other DNA
cccctgtccagcaggaaacc

SEQ ID NO: 434 - DNA, 20 bp, synthetic construct
  misc_feature 1..20: note = Synthetic Oligonucleotide
  source 1..20: mol_type = other DNA
cgcccctgtccagcaggaaa

SEQ ID NO: 435 - DNA, 20 bp, synthetic construct
  misc_feature 1..20: note = Synthetic Oligonucleotide
  source 1..20: mol_type = other DNA
acgcccctgtccagcaggaa

SEQ ID NO: 436 - DNA, 20 bp, synthetic construct
  misc_feature 1..20: note = Synthetic Oligonucleotide
  source 1..20: mol_type = other DNA
cacgcccctgtccagcagga

SEQ ID NO: 437 - DNA, 20 bp, synthetic construct
  misc_feature 1..20: note = Synthetic Oligonucleotide
  source 1..20: mol_type = other DNA
ccacgcccctgtccagcagg

SEQ ID NO: 438 - DNA, 20 bp, synthetic construct
  misc_feature 1..20: note = Synthetic Oligonucleotide
  source 1..20: mol_type = other DNA
cccacgcccctgtccagcag

SEQ ID NO: 439 - DNA, 20 bp, synthetic construct
  misc_feature 1..20: note = Synthetic Oligonucleotide
  source 1..20: mol_type = other DNA
tcccacgcccctgtccagca

SEQ ID NO: 440 - DNA, 20 bp, synthetic construct
  misc_feature 1..20: note = Synthetic Oligonucleotide
  source 1..20: mol_type = other DNA
atcccacgcccctgtccagc

SEQ ID NO: 441 - DNA, 20 bp, synthetic construct
  misc_feature 1..20: note = Synthetic Oligonucleotide
  source 1..20: mol_type = other DNA
aatcccacgcccctgtccag

SEQ ID NO: 442 - DNA, 20 bp, synthetic construct
  misc_feature 1..20: note = Synthetic Oligonucleotide
  source 1..20: mol_type = other DNA
caatcccacgcccctgtcca

SEQ ID NO: 443 - DNA, 20 bp, synthetic construct
  misc_feature 1..20: note = Synthetic Oligonucleotide
  source 1..20: mol_type = other DNA
tcaatcccacgcccctgtcc

SEQ ID NO: 444 - DNA, 20 bp, synthetic construct
  misc_feature 1..20: note = Synthetic Oligonucleotide
  source 1..20: mol_type = other DNA
ttcaatcccacgcccctgtc

SEQ ID NO: 445 - DNA, 20 bp, synthetic construct
  misc_feature 1..20: note = Synthetic Oligonucleotide
  source 1..20: mol_type = other DNA
attcaatcccacgcccctgt

SEQ ID NO: 446 - DNA, 20 bp, synthetic construct
  misc_feature 1..20: note = Synthetic Oligonucleotide
  source 1..20: mol_type = other DNA
aattcaatcccacgcccctg

SEQ ID NO: 447 - DNA, 20 bp, synthetic construct
  misc_feature 1..20: note = Synthetic Oligonucleotide
  source 1..20: mol_type = other DNA
taattcaatcccacgcccct

SEQ ID NO: 448 - DNA, 20 bp, synthetic construct
  misc_feature 1..20: note = Synthetic Oligonucleotide
  source 1..20: mol_type = other DNA
ttaattcaatcccacgcccc

SEQ ID NO: 449 - DNA, 20 bp, synthetic construct
  misc_feature 1..20: note = Synthetic Oligonucleotide
  source 1..20: mol_type = other DNA
tttaattcaatcccacgccc

SEQ ID NO: 450 - DNA, 20 bp, synthetic construct
  misc_feature 1..20: note = Synthetic Oligonucleotide
  source 1..20: mol_type = other DNA
ttttaattcaatcccacgcc

SEQ ID NO: 451 - DNA, 20 bp, synthetic construct
  misc_feature 1..20: note = Synthetic Oligonucleotide
  source 1..20: mol_type = other DNA
gttttaattcaatcccacgc

SEQ ID NO: 452 - DNA, 20 bp, synthetic construct
  misc_feature 1..20: note = Synthetic Oligonucleotide
  source 1..20: mol_type = other DNA
tgttttaattcaatcccacg

SEQ ID NO: 453 - DNA, 20 bp, synthetic construct
  misc_feature 1..20: note = Synthetic Oligonucleotide
  source 1..20: mol_type = other DNA
ctgttttaattcaatcccac

SEQ ID NO: 454 - DNA, 20 bp, synthetic construct
  misc_feature 1..20: note = Synthetic Oligonucleotide
  source 1..20: mol_type = other DNA
gctgttttaattcaatccca

SEQ ID NO: 455 - DNA, 20 bp, synthetic construct
  misc_feature 1..20: note = Synthetic Oligonucleotide
  source 1..20: mol_type = other DNA
cagctgttttaattcaatcc

SEQ ID NO: 456 - DNA, 20 bp, synthetic construct
  misc_feature 1..20: note = Synthetic Oligonucleotide
  source 1..20: mol_type = other DNA
gcagctgttttaattcaatc

SEQ ID NO: 457 - DNA, 20 bp, synthetic construct
  misc_feature 1..20: note = Synthetic Oligonucleotide
  source 1..20: mol_type = other DNA
cgcagctgttttaattcaat

SEQ ID NO: 458 - DNA, 20 bp, synthetic construct
  misc_feature 1..20: note = Synthetic Oligonucleotide
  source 1..20: mol_type = other DNA
tcgcagctgttttaattcaa

SEQ ID NO: 459 - DNA, 20 bp, synthetic construct
  misc_feature 1..20: note = Synthetic Oligonucleotide
  source 1..20: mol_type = other DNA
tgtcgcagctgttttaattc

SEQ ID NO: 460 - DNA, 20 bp, synthetic construct
  misc_feature 1..20: note = Synthetic Oligonucleotide
  source 1..20: mol_type = other DNA
ttgtcgcagctgttttaatt

SEQ ID NO: 461 - DNA, 20 bp, synthetic construct
  misc_feature 1..20: note = Synthetic Oligonucleotide
  source 1..20: mol_type = other DNA
gttgtcgcagctgttttaat

SEQ ID NO: 462 - DNA, 20 bp, synthetic construct
  misc_feature 1..20: note = Synthetic Oligonucleotide
  source 1..20: mol_type = other DNA
ttgttgtcgcagctgtttta

SEQ ID NO: 463 - DNA, 20 bp, synthetic construct
  misc_feature 1..20: note = Synthetic Oligonucleotide
  source 1..20: mol_type = other DNA
tttgttgtcgcagctgtttt

SEQ ID NO: 464 - DNA, 20 bp, synthetic construct
  misc_feature 1..20: note = Synthetic Oligonucleotide
  source 1..20: mol_type = other DNA
ttttgttgtcgcagctgttt

SEQ ID NO: 465 - DNA, 20 bp, synthetic construct
  misc_feature 1..20: note = Synthetic Oligonucleotide
  source 1..20: mol_type = other DNA
tttttgttgtcgcagctgtt

SEQ ID NO: 466 - DNA, 20 bp, synthetic construct
  misc_feature 1..20: note = Synthetic Oligonucleotide
  source 1..20: mol_type = other DNA
ggatccagctcactcccctg

SEQ ID NO: 467 - DNA, 20 bp, synthetic construct
  misc_feature 1..20: note = Synthetic Oligonucleotide
  source 1..20: mol_type = other DNA
aaataaggatccagctcact

SEQ ID NO: 468 - DNA, 20 bp, synthetic construct
  misc_feature 1..20: note = Synthetic Oligonucleotide
  source 1..20: mol_type = other DNA
gaccagaaataaggatccag

SEQ ID NO: 469 - DNA, 20 bp, synthetic construct
  misc_feature 1..20: note = Synthetic Oligonucleotide
  source 1..20: mol_type = other DNA
cttagggaccagaaataagg

SEQ ID NO: 470 - DNA, 20 bp, synthetic construct
  misc_feature 1..20: note = Synthetic Oligonucleotide
  source 1..20: mol_type = other DNA
cacccacttagggaccagaa

SEQ ID NO: 471 - DNA, 20 bp, synthetic construct
  misc_feature 1..20: note = Synthetic Oligonucleotide
  source 1..20: mol_type = other DNA
accacccacttagggaccag

SEQ ID NO: 472 - DNA, 20 bp, synthetic construct
  misc_feature 1..20: note = Synthetic Oligonucleotide
  source 1..20: mol_type = other DNA
aggtccaggactctcccctt

SEQ ID NO: 473 - DNA, 20 bp, synthetic construct
  misc_feature 1..20: note = Synthetic Oligonucleotide
  source 1..20: mol_type = other DNA
aaggtccaggactctcccct

SEQ ID NO: 474 - DNA, 20 bp, synthetic construct
  misc_feature 1..20: note = Synthetic Oligonucleotide
  source 1..20: mol_type = other DNA
aaactgcagaagtcccaccc

SEQ ID NO: 475 - DNA, 20 bp, synthetic construct
  misc_feature 1..20: note = Synthetic Oligonucleotide
  source 1..20: mol_type = other DNA
ggagggccccgctgagctgc

SEQ ID NO: 476 - DNA, 20 bp, synthetic construct
  misc_feature 1..20: note = Synthetic Oligonucleotide
  source 1..20: mol_type = other DNA
tcccggaacatccaagcggg

SEQ ID NO: 477 - DNA, 20 bp, synthetic construct
  misc_feature 1..20: note = Synthetic Oligonucleotide
  source 1..20: mol_type = other DNA
catcactttcccggaacatc

SEQ ID NO: 478 - DNA, 20 bp, synthetic construct
  misc_feature 1..20: note = Synthetic Oligonucleotide
  source 1..20: mol_type = other DNA
ctggtcacattcccttcccc

SEQ ID NO: 479 - DNA, 20 bp, synthetic construct
  misc_feature 1..20: note = Synthetic Oligonucleotide
  source 1..20: mol_type = other DNA
ctagacctggtcacattccc

SEQ ID NO: 480 - DNA, 20 bp, synthetic construct
  misc_feature 1..20: note = Synthetic Oligonucleotide
  source 1..20: mol_type = other DNA
ggagtggtggtcacacctcc

SEQ ID NO: 481 - DNA, 20 bp, synthetic construct
  misc_feature 1..20: note = Synthetic Oligonucleotide
  source 1..20: mol_type = other DNA
accccctccagagagcagga

SEQ ID NO: 482 - DNA, 20 bp, synthetic construct
  misc_feature 1..20: note = Synthetic Oligonucleotide
  source 1..20: mol_type = other DNA
atctctaccccctccagaga

SEQ ID NO: 483 - DNA, 20 bp, synthetic construct
  misc_feature 1..20: note = Synthetic Oligonucleotide
  source 1..20: mol_type = other DNA
ggtacgggtagaagccagaa

SEQ ID NO: 484 - DNA, 20 bp, synthetic construct
  misc_feature 1..20: note = Synthetic Oligonucleotide
  source 1..20: mol_type = other DNA
ggagagtgtaaccgtcatag

SEQ ID NO: 485 - DNA, 20 bp, synthetic construct
  misc_feature 1..20: note = Synthetic Oligonucleotide
  source 1..20: mol_type = other DNA
tgcgattggcagagccccgg

SEQ ID NO: 486 - DNA, 20 bp, synthetic construct
  misc_feature 1..20: note = Synthetic Oligonucleotide
  source 1..20: mol_type = other DNA
ggcaggtgcgattggcagag

SEQ ID NO: 487 - DNA, 20 bp, synthetic construct
  misc_feature 1..20: note = Synthetic Oligonucleotide
  source 1..20: mol_type = other DNA
ggccattcacttggcaggtg

SEQ ID NO: 488 - DNA, 20 bp, synthetic construct
  misc_feature 1..20: note = Synthetic Oligonucleotide
  source 1..20: mol_type = other DNA
ttgtcacagatcgctgtctg

SEQ ID NO: 489 - DNA, 20 bp, synthetic construct
  misc_feature 1..20: note = Synthetic Oligonucleotide
  source 1..20: mol_type = other DNA
aaggagtcttggcaggaagg

SEQ ID NO: 490 - DNA, 20 bp, synthetic construct
  misc_feature 1..20: note = Synthetic Oligonucleotide
  source 1..20: mol_type = other DNA
gtacatgaaggagtcttggc

SEQ ID NO: 491 - DNA, 20 bp, synthetic construct
  misc_feature 1..20: note = Synthetic Oligonucleotide
  source 1..20: mol_type = other DNA
aagcttcggccacctcttga

SEQ ID NO: 492 - DNA, 20 bp, synthetic construct
  misc_feature 1..20: note = Synthetic Oligonucleotide
  source 1..20: mol_type = other DNA
ccatctagcaccaggtagat

SEQ ID NO: 493 - DNA, 20 bp, synthetic construct
  misc_feature 1..20: note = Synthetic Oligonucleotide
  source 1..20: mol_type = other DNA
ggccccaatgctgtctgatc

SEQ ID NO: 494 - DNA, 20 bp, synthetic construct
  misc_feature 1..20: note = Synthetic Oligonucleotide
  source 1..20: mol_type = other DNA
aattaagttgactagacact

SEQ ID NO: 495 - DNA, 20 bp, synthetic construct
  misc_feature 1..20: note = Synthetic Oligonucleotide
  source 1..20: mol_type = other DNA
tgccaccttctcaattaagt

SEQ ID NO: 496 - DNA, 20 bp, synthetic construct
  misc_feature 1..20: note = Synthetic Oligonucleotide
  source 1..20: mol_type = other DNA
taacttgccaccttctcaat

SEQ ID NO: 497 - DNA, 20 bp, synthetic construct
  misc_feature 1..20: note = Synthetic Oligonucleotide
  source 1..20: mol_type = other DNA
cataacttgccaccttctca

SEQ ID NO: 498 - DNA, 20 bp, synthetic construct
  misc_feature 1..20: note = Synthetic Oligonucleotide
  source 1..20: mol_type = other DNA
acaccataacttgccacctt

SEQ ID NO: 499 - DNA, 20 bp, synthetic construct
  misc_feature 1..20: note = Synthetic Oligonucleotide
  source 1..20: mol_type = other DNA
tcacaccataacttgccacc

SEQ ID NO: 500 - DNA, 20 bp, synthetic construct
  misc_feature 1..20: note = Synthetic Oligonucleotide
  source 1..20: mol_type = other DNA
tagtccctgacttcaacttg

SEQ ID NO: 501 - DNA, 20 bp, synthetic construct
  misc_feature 1..20: note = Synthetic Oligonucleotide
  source 1..20: mol_type = other DNA
tggtgttagtccctgacttc

SEQ ID NO: 502 - DNA, 20 bp, synthetic construct
  misc_feature 1..20: note = Synthetic Oligonucleotide
  source 1..20: mol_type = other DNA
gcggttccagccttcaggag

SEQ ID NO: 503 - DNA, 20 bp, synthetic construct
  misc_feature 1..20: note = Synthetic Oligonucleotide
  source 1..20: mol_type = other DNA
tcatgaggatgatgacatgg

SEQ ID NO: 504 - DNA, 20 bp, synthetic construct
  misc_feature 1..20: note = Synthetic Oligonucleotide
  source 1..20: mol_type = other DNA
ccgcccatgttgtgcaatcc

SEQ ID NO: 505 - DNA, 20 bp, synthetic construct
  misc_feature 1..20: note = Synthetic Oligonucleotide
  source 1..20: mol_type = other DNA
gtaattgggtccccgcccat

SEQ ID NO: 506 - DNA, 20 bp, synthetic construct
  misc_feature 1..20: note = Synthetic Oligonucleotide
  source 1..20: mol_type = other DNA
aagtcccggatctcatcaat

SEQ ID NO: 507 - DNA, 20 bp, synthetic construct
  misc_feature 1..20: note = Synthetic Oligonucleotide
  source 1..20: mol_type = other DNA
aacacatagacatccagata

SEQ ID NO: 508 - DNA, 20 bp, synthetic construct
  misc_feature 1..20: note = Synthetic Oligonucleotide
  source 1..20: mol_type = other DNA
caaagcattgatgttcactt

SEQ ID NO: 509 - DNA, 20 bp, synthetic construct
  misc_feature 1..20: note = Synthetic Oligonucleotide
  source 1..20: mol_type = other DNA
tttgaacacatgttgctcat

SEQ ID NO: 510 - DNA, 20 bp, synthetic construct
  misc_feature 1..20: note = Synthetic Oligonucleotide
  source 1..20: mol_type = other DNA
cttccaggttttccatatcc

SEQ ID NO: 511 - DNA, 20 bp, synthetic construct
  misc_feature 1..20: note = Synthetic Oligonucleotide
  source 1..20: mol_type = other DNA
tcttccaggttttccatatc

SEQ ID NO: 512 - DNA, 20 bp, synthetic construct
  misc_feature 1..20: note = Synthetic Oligonucleotide
  source 1..20: mol_type = other DNA
agactcagagactggctttc

SEQ ID NO: 513 - DNA, 20 bp, synthetic construct
  misc_feature 1..20: note = Synthetic Oligonucleotide
  source 1..20: mol_type = other DNA
gcctgccatggttgcttgtg

SEQ ID NO: 514 - DNA, 20 bp, synthetic construct
  misc_feature 1..20: note = Synthetic Oligonucleotide
  source 1..20: mol_type = other DNA
tgactgagatcttggcctgc

SEQ ID NO: 515 - DNA, 20 bp, synthetic construct
  misc_feature 1..20: note = Synthetic Oligonucleotide
  source 1..20: mol_type = other DNA
ttctatctccaggtcccgct

SEQ ID NO: 516 - DNA, 20 bp, synthetic construct
  misc_feature 1..20: note = Synthetic Oligonucleotide
  source 1..20: mol_type = other DNA
agtcataaaattcaggaatt

SEQ ID NO: 517 - DNA, 20 bp, synthetic construct
  misc_feature 1..20: note = Synthetic Oligonucleotide
  source 1..20: mol_type = other DNA
cgagttgttccctcggtgca

SEQ ID NO: 518 - DNA, 20 bp, synthetic construct
  misc_feature 1..20: note = Synthetic Oligonucleotide
  source 1..20: mol_type = other DNA
agcctcaaagctcgagttgt

SEQ ID NO: 519 - DNA, 20 bp, synthetic construct
  misc_feature 1..20: note = Synthetic Oligonucleotide
  source 1..20: mol_type = other DNA
ggaggaagcctcaaagctcg

SEQ ID NO: 520 - DNA, 20 bp, synthetic construct
  misc_feature 1..20: note = Synthetic Oligonucleotide
  source 1..20: mol_type = other DNA
gtagttggaggaagcctcaa

SEQ ID NO: 521 - DNA, 20 bp, synthetic construct
  misc_feature 1..20: note = Synthetic Oligonucleotide
  source 1..20: mol_type = other DNA
caagtggtagttggaggaag

SEQ ID NO: 522 - DNA, 20 bp, synthetic construct
  misc_feature 1..20: note = Synthetic Oligonucleotide
  source 1..20: mol_type = other DNA
tcctcagacacaaacagagc

SEQ ID NO: 523 - DNA, 20 bp, synthetic construct
  misc_feature 1..20: note = Synthetic Oligonucleotide
  source 1..20: mol_type = other DNA
ttctcctcctcagacacaaa

SEQ ID NO: 524 - DNA, 20 bp, synthetic construct
  misc_feature 1..20: note = Synthetic Oligonucleotide
  source 1..20: mol_type = other DNA
tagacctccttccgagtcag

SEQ ID NO: 525 - DNA, 20 bp, synthetic construct
  misc_feature 1..20: note = Synthetic Oligonucleotide
  source 1..20: mol_type = other DNA
ttgatgtagacctccttccg

SEQ ID NO: 526 - DNA, 20 bp, synthetic construct
  misc_feature 1..20: note = Synthetic Oligonucleotide
  source 1..20: mol_type = other DNA
ctttcttatccccattcttg

SEQ ID NO: 527 - DNA, 20 bp, synthetic construct
  misc_feature 1..20: note = Synthetic Oligonucleotide
  source 1..20: mol_type = other DNA
gcctttcttatccccattct

SEQ ID NO: 528 - DNA, 20 bp, synthetic construct
  misc_feature 1..20: note = Synthetic Oligonucleotide
  source 1..20: mol_type = other DNA
agctgcctttcttatcccca

SEQ ID NO: 529 - DNA, 20 bp, synthetic construct
  misc_feature 1..20: note = Synthetic Oligonucleotide
  source 1..20: mol_type = other DNA
cagctgcctttcttatcccc

SEQ ID NO: 530 - DNA, 20 bp, synthetic construct
  misc_feature 1..20: note = Synthetic Oligonucleotide
  source 1..20: mol_type = other DNA
acagctgcctttcttatccc

SEQ ID NO: 531 - DNA, 20 bp, synthetic construct
  misc_feature 1..20: note = Synthetic Oligonucleotide
  source 1..20: mol_type = other DNA
gcatctctctcacagctgcc

SEQ ID NO: 532 - DNA, 20 bp, synthetic construct
  misc_feature 1..20: note = Synthetic Oligonucleotide
  source 1..20: mol_type = other DNA
agatgtccttgactttgtca

SEQ ID NO: 533 - DNA, 20 bp, synthetic construct
  misc_feature 1..20: note = Synthetic Oligonucleotide
  source 1..20: mol_type = other DNA
cagcatagggactcactcct

SEQ ID NO: 534 - DNA, 20 bp, synthetic construct
  misc_feature 1..20: note = Synthetic Oligonucleotide
  source 1..20: mol_type = other DNA
ccgccagaatcacctctgca

SEQ ID NO: 535 - DNA, 20 bp, synthetic construct
  misc_feature 1..20: note = Synthetic Oligonucleotide
  source 1..20: mol_type = other DNA
tgaatgaaacgacttctctt

SEQ ID NO: 536 - DNA, 20 bp, synthetic construct
  misc_feature 1..20: note = Synthetic Oligonucleotide
  source 1..20: mol_type = other DNA
acatccactactccccagct

SEQ ID NO: 537 - DNA, 20 bp, synthetic construct
  misc_feature 1..20: note = Synthetic Oligonucleotide
  source 1..20: mol_type = other DNA
cgcttctggtttttgcagac

SEQ ID NO: 538 - DNA, 20 bp, synthetic construct
  misc_feature 1..20: note = Synthetic Oligonucleotide
  source 1..20: mol_type = other DNA
ttttgccgcttctggttttt

SEQ ID NO: 539 - DNA, 20 bp, synthetic construct
  misc_feature 1..20: note = Synthetic Oligonucleotide
  source 1..20: mol_type = other DNA
gcaggtacctgcttttgccg

SEQ ID NO: 540 - DNA, 20 bp, synthetic construct
  misc_feature 1..20: note = Synthetic Oligonucleotide
  source 1..20: mol_type = other DNA
tcttggagtttctccttcag

SEQ ID NO: 541 - DNA, 16 bp, synthetic construct
  misc_feature 1..16: note = Synthetic Oligonucleotide
  source 1..16: mol_type = other DNA
ggaacatccaagcggg

SEQ ID NO: 542 - DNA, 16 bp, synthetic construct
  misc_feature 1..16: note = Synthetic Oligonucleotide
  source 1..16: mol_type = other DNA
tggtcacattcccttc

SEQ ID NO: 543 - DNA, 16 bp, synthetic construct
  misc_feature 1..16: note = Synthetic Oligonucleotide
  source 1..16: mol_type = other DNA
cctggtcacattccct

SEQ ID NO: 544 - DNA, 16 bp, synthetic construct
  misc_feature 1..16: note = Synthetic Oligonucleotide
  source 1..16: mol_type = other DNA
gacctggtcacattcc

SEQ ID NO: 545 - DNA, 16 bp, synthetic construct
  misc_feature 1..16: note = Synthetic Oligonucleotide
  source 1..16: mol_type = other DNA
taacttgccaccttct

SEQ ID NO: 546 - DNA, 16 bp, synthetic construct
  misc_feature 1..16: note = Synthetic Oligonucleotide
  source 1..16: mol_type = other DNA
cataacttgccacctt

SEQ ID NO: 547 - DNA, 16 bp, synthetic construct
  misc_feature 1..16: note = Synthetic Oligonucleotide
  source 1..16: mol_type = other DNA
accataacttgccacc

SEQ ID NO: 548 - DNA, 16 bp, synthetic construct
  misc_feature 1..16: note = Synthetic Oligonucleotide
  source 1..16: mol_type = other DNA
ccttccgagtcagctt

SEQ ID NO: 549 - DNA, 16 bp, synthetic construct
  misc_feature 1..16: note = Synthetic Oligonucleotide
  source 1..16: mol_type = other DNA
ctccttccgagtcagc

SEQ ID NO: 550 - DNA, 16 bp, synthetic construct
  misc_feature 1..16: note = Synthetic Oligonucleotide
  source 1..16: mol_type = other DNA
acctccttccgagtca

SEQ ID NO: 551 - DNA, 16 bp, synthetic construct
  misc_feature 1..16: note = Synthetic Oligonucleotide
  source 1..16: mol_type = other DNA
ctttcttatccccatt

SEQ ID NO: 552 - DNA, 16 bp, synthetic construct
  misc_feature 1..16: note = Synthetic Oligonucleotide
  source 1..16: mol_type = other DNA
gcctttcttatcccca

SEQ ID NO: 553 - DNA, 16 bp, synthetic construct
  misc_feature 1..16: note = Synthetic Oligonucleotide
  source 1..16: mol_type = other DNA
ctgcctttcttatccc

SEQ ID NO: 554 - DNA, 16 bp, synthetic construct
  misc_feature 1..16: note = Synthetic Oligonucleotide
  source 1..16: mol_type = other DNA
tttgccgcttctggtt

SEQ ID NO: 555 - DNA, 16 bp, synthetic construct
  misc_feature 1..16: note = Synthetic Oligonucleotide
  source 1..16: mol_type = other DNA
cttttgccgcttctgg

SEQ ID NO: 556 - DNA, 16 bp, synthetic construct
  misc_feature 1..16: note = Synthetic Oligonucleotide
  source 1..16: mol_type = other DNA
tgcttttgccgcttct

SEQ ID NO: 557 - DNA, 16 bp, synthetic construct
  misc_feature 1..16: note = Synthetic Oligonucleotide
  source 1..16: mol_type = other DNA
aaacccaaatcctcat

SEQ ID NO: 558 - DNA, 16 bp, synthetic construct
  misc_feature 1..16: note = Synthetic Oligonucleotide
  source 1..16: mol_type = other DNA
gaaaacccaaatcctc

SEQ ID NO: 559 - DNA, 16 bp, synthetic construct
  misc_feature 1..16: note = Synthetic Oligonucleotide
  source 1..16: mol_type = other DNA
tagaaaacccaaatcc

SEQ ID NO: 560 - DNA, 16 bp, synthetic construct
  misc_feature 1..16: note = Synthetic Oligonucleotide
  source 1..16: mol_type = other DNA
atagaaaacccaaatc

SEQ ID NO: 561 - DNA, 16 bp, synthetic construct
  misc_feature 1..16: note = Synthetic Oligonucleotide
  source 1..16: mol_type = other DNA
cttatagaaaacccaa

SEQ ID NO: 562 - DNA, 16 bp, synthetic construct
  misc_feature 1..16: note = Synthetic Oligonucleotide
  source 1..16: mol_type = other DNA
ccttatagaaaaccca

SEQ ID NO: 563 - DNA, 16 bp, synthetic construct
  misc_feature 1..16: note = Synthetic Oligonucleotide
  source 1..16: mol_type = other DNA
cccttatagaaaaccc

SEQ ID NO: 564 - DNA, 16 bp, synthetic construct
  misc_feature 1..16: note = Synthetic Oligonucleotide
  source 1..16: mol_type = other DNA
ccccttatagaaaacc

SEQ ID NO: 565 - DNA, 16 bp, synthetic construct
  misc_feature 1..16: note = Synthetic Oligonucleotide
  source 1..16: mol_type = other DNA
accccttatagaaaac

SEQ ID NO: 566 - DNA, 16 bp, synthetic construct
  misc_feature 1..16: note = Synthetic Oligonucleotide
  source 1..16: mol_type = other DNA
aaccccttatagaaaa

SEQ ID NO: 567 - DNA, 16 bp, synthetic construct
  misc_feature 1..16: note = Synthetic Oligonucleotide
  source 1..16: mol_type = other DNA
aaaccccttatagaaa

SEQ ID NO: 568 - DNA, 16 bp, synthetic construct
  misc_feature 1..16: note = Synthetic Oligonucleotide
  source 1..16: mol_type = other DNA
gaaaccccttatagaa

SEQ ID NO: 569 - DNA, 16 bp, synthetic construct
  misc_feature 1..16: note = Synthetic Oligonucleotide
  source 1..16: mol_type = other DNA
ggaaaccccttataga

SEQ ID NO: 570 - DNA, 16 bp, synthetic construct
  misc_feature 1..16: note = Synthetic Oligonucleotide
  source 1..16: mol_type = other DNA
aggaaaccccttatag

SEQ ID NO: 571 - DNA, 16 bp, synthetic construct
  misc_feature 1..16: note = Synthetic Oligonucleotide
  source 1..16: mol_type = other DNA
caggaaaccccttata

SEQ ID NO: 572 - DNA, 16 bp, synthetic construct
  misc_feature 1..16: note = Synthetic Oligonucleotide
  source 1..16: mol_type = other DNA
gcaggaaaccccttat

SEQ ID NO: 573 - DNA, 16 bp, synthetic construct
  misc_feature 1..16: note = Synthetic Oligonucleotide
  source 1..16: mol_type = other DNA
agcaggaaacccctta

SEQ ID NO: 574 - DNA, 16 bp, synthetic construct
  misc_feature 1..16: note = Synthetic Oligonucleotide
  source 1..16: mol_type = other DNA
cagcaggaaacccctt

SEQ ID NO: 575 - DNA, 16 bp, synthetic construct
  misc_feature 1..16: note = Synthetic Oligonucleotide
  source 1..16: mol_type = other DNA
ccagcaggaaacccct

SEQ ID NO: 576 - DNA, 16 bp, synthetic construct
  misc_feature 1..16: note = Synthetic Oligonucleotide
  source 1..16: mol_type = other DNA
tccagcaggaaacccc

SEQ ID NO: 577 - DNA, 16 bp, synthetic construct
  misc_feature 1..16: note = Synthetic Oligonucleotide
  source 1..16: mol_type = other DNA
gtccagcaggaaaccc

SEQ ID NO: 578 - DNA, 16 bp, synthetic construct
  misc_feature 1..16: note = Synthetic Oligonucleotide
  source 1..16: mol_type = other DNA
tgtccagcaggaaacc

SEQ ID NO: 579 - DNA, 16 bp, synthetic construct
  misc_feature 1..16: note = Synthetic Oligonucleotide
  source 1..16: mol_type = other DNA
ctgtccagcaggaaac

SEQ ID NO: 580 - DNA, 16 bp, synthetic construct
  misc_feature 1..16: note = Synthetic Oligonucleotide
  source 1..16: mol_type = other DNA
cctgtccagcaggaaa

SEQ ID NO: 581 - DNA, 16 bp, synthetic construct
  misc_feature 1..16: note = Synthetic Oligonucleotide
  source 1..16: mol_type = other DNA
ccctgtccagcaggaa

SEQ ID NO: 582 - DNA, 16 bp, synthetic construct
  misc_feature 1..16: note = Synthetic Oligonucleotide
  source 1..16: mol_type = other DNA
cccctgtccagcagga

SEQ ID NO: 583 - DNA, 16 bp, synthetic construct
  misc_feature 1..16: note = Synthetic Oligonucleotide
  source 1..16: mol_type = other DNA
gcccctgtccagcagg

SEQ ID NO: 584 - DNA, 16 bp, synthetic construct
  misc_feature 1..16: note = Synthetic Oligonucleotide
  source 1..16: mol_type = other DNA
cgcccctgtccagcag

SEQ ID NO: 585 - DNA, 16 bp, synthetic construct
  misc_feature 1..16: note = Synthetic Oligonucleotide
  source 1..16: mol_type = other DNA
acgcccctgtccagca

SEQ ID NO: 586 - DNA, 16 bp, synthetic construct
  misc_feature 1..16: note = Synthetic Oligonucleotide
  source 1..16: mol_type = other DNA
cacgcccctgtccagc

SEQ ID NO: 587 - DNA, 16 bp, synthetic construct
  misc_feature 1..16: note = Synthetic Oligonucleotide
  source 1..16: mol_type = other DNA
ccacgcccctgtccag

SEQ ID NO: 588 - DNA, 16 bp, synthetic construct
  misc_feature 1..16: note = Synthetic Oligonucleotide
  source 1..16: mol_type = other DNA
cccacgcccctgtcca

SEQ ID NO: 589 - DNA, 16 bp, synthetic construct
  misc_feature 1..16: note = Synthetic Oligonucleotide
  source 1..16: mol_type = other DNA
tcccacgcccctgtcc

SEQ ID NO: 590 - DNA, 16 bp, synthetic construct
  misc_feature 1..16: note = Synthetic Oligonucleotide
  source 1..16: mol_type = other DNA
atcccacgcccctgtc

SEQ ID NO: 591 - DNA, 16 bp, synthetic construct
  misc_feature 1..16: note = Synthetic Oligonucleotide
  source 1..16: mol_type = other DNA
aatcccacgcccctgt

SEQ ID NO: 592 - DNA, 16 bp, synthetic construct
  misc_feature 1..16: note = Synthetic Oligonucleotide
  source 1..16: mol_type = other DNA
caatcccacgcccctg

SEQ ID NO: 593 - DNA, 16 bp, synthetic construct
  misc_feature 1..16: note = Synthetic Oligonucleotide
  source 1..16: mol_type = other DNA
tcaatcccacgcccct

SEQ ID NO: 594 - DNA, 16 bp, synthetic construct
  misc_feature 1..16: note = Synthetic Oligonucleotide
  source 1..16: mol_type = other DNA
ttcaatcccacgcccc

SEQ ID NO: 595 - DNA, 16 bp, synthetic construct
  misc_feature 1..16: note = Synthetic Oligonucleotide
  source 1..16: mol_type = other DNA
attcaatcccacgccc

SEQ ID NO: 596 - DNA, 16 bp, synthetic construct
  misc_feature 1..16: note = Synthetic Oligonucleotide
  source 1..16: mol_type = other DNA
aattcaatcccacgcc

SEQ ID NO: 597 - DNA, 16 bp, synthetic construct
  misc_feature 1..16: note = Synthetic Oligonucleotide
  source 1..16: mol_type = other DNA
taattcaatcccacgc

SEQ ID NO: 598 - DNA, 16 bp, synthetic construct
  misc_feature 1..16: note = Synthetic Oligonucleotide
  source 1..16: mol_type = other DNA
ttaattcaatcccacg

SEQ ID NO: 599 - DNA, 16 bp, synthetic construct
  misc_feature 1..16: note = Synthetic Oligonucleotide
  source 1..16: mol_type = other DNA
tttaattcaatcccac

SEQ ID NO: 600 - DNA, 16 bp, synthetic construct
  misc_feature 1..16: note = Synthetic Oligonucleotide
  source 1..16: mol_type = other DNA
ttttaattcaatccca

SEQ ID NO: 601 - DNA, 16 bp, synthetic construct
  misc_feature 1..16: note = Synthetic Oligonucleotide
  source 1..16: mol_type = other DNA
gttttaattcaatccc

SEQ ID NO: 602 - DNA, 16 bp, synthetic construct
  misc_feature 1..16: note = Synthetic Oligonucleotide
  source 1..16: mol_type = other DNA
tgttttaattcaatcc

SEQ ID NO: 603 - DNA, 16 bp, synthetic construct
  misc_feature 1..16: note = Synthetic Oligonucleotide
  source 1..16: mol_type = other DNA
ctgttttaattcaatc

SEQ ID NO: 604 - DNA, 16 bp, synthetic construct
  misc_feature 1..16: note = Synthetic Oligonucleotide
  source 1..16: mol_type = other DNA
gctgttttaattcaat

SEQ ID NO: 605 - DNA, 16 bp, synthetic construct
  misc_feature 1..16: note = Synthetic Oligonucleotide
  source 1..16: mol_type = other DNA
agctgttttaattcaa

SEQ ID NO: 606 - DNA, 16 bp, synthetic construct
  misc_feature 1..16: note = Synthetic Oligonucleotide
  source 1..16: mol_type = other DNA
cagctgttttaattca

SEQ ID NO: 607 - DNA, 16 bp, synthetic construct
  misc_feature 1..16: note = Synthetic Oligonucleotide
  source 1..16: mol_type = other DNA
gcagctgttttaattc

SEQ ID NO: 608 - DNA, 16 bp, synthetic construct
  misc_feature 1..16: note = Synthetic Oligonucleotide
  source 1..16: mol_type = other DNA
cgcagctgttttaatt

SEQ ID NO: 609 - DNA, 16 bp, synthetic construct
  misc_feature 1..16: note = Synthetic Oligonucleotide
  source 1..16: mol_type = other DNA
tcgcagctgttttaat

SEQ ID NO: 610 - DNA, 16 bp, synthetic construct
  misc_feature 1..16: note = Synthetic Oligonucleotide
  source 1..16: mol_type = other DNA
gtcgcagctgttttaa

SEQ ID NO: 611 - DNA, 16 bp, synthetic construct
  misc_feature 1..16: note = Synthetic Oligonucleotide
  source 1..16: mol_type = other DNA
tgtcgcagctgtttta

SEQ ID NO: 612 - DNA, 16 bp, synthetic construct
  misc_feature 1..16: note = Synthetic Oligonucleotide
  source 1..16: mol_type = other DNA
ttgtcgcagctgtttt

SEQ ID NO: 613 - DNA, 16 bp, synthetic construct
  misc_feature 1..16: note = Synthetic Oligonucleotide
  source 1..16: mol_type = other DNA
gttgtcgcagctgttt

SEQ ID NO: 614 - DNA, 16 bp, synthetic construct
  misc_feature 1..16: note = Synthetic Oligonucleotide
  source 1..16: mol_type = other DNA
tgttgtcgcagctgtt

SEQ ID NO: 615 - DNA, 16 bp, synthetic construct
  misc_feature 1..16: note = Synthetic Oligonucleotide
  source 1..16: mol_type = other DNA
ttgttgtcgcagctgt

SEQ ID NO: 616 - DNA, 16 bp, synthetic construct
  misc_feature 1..16: note = Synthetic Oligonucleotide
  source 1..16: mol_type = other DNA
tttgttgtcgcagctg

SEQ ID NO: 617 - DNA, 16 bp, synthetic construct
  misc_feature 1..16: note = Synthetic Oligonucleotide
  source 1..16: mol_type = other DNA
ttttgttgtcgcagct

SEQ ID NO: 618 - DNA, 16 bp, synthetic construct
  misc_feature 1..16: note = Synthetic Oligonucleotide
  source 1..16: mol_type = other DNA
tttttgttgtcgcagc

SEQ ID NO: 619 - DNA, 17 bp, synthetic construct
  misc_feature 1..17: note = Synthetic Oligonucleotide
  source 1..17: mol_type = other DNA
gaaaacccaaatcctca

SEQ ID NO: 620 - DNA, 17 bp, synthetic construct
  misc_feature 1..17: note = Synthetic Oligonucleotide
  source 1..17: mol_type = other DNA
agaaaacccaaatcctc

SEQ ID NO: 621 - DNA, 17 bp, synthetic construct
  misc_feature 1..17: note = Synthetic Oligonucleotide
  source 1..17: mol_type = other DNA
tagaaaacccaaatcct

SEQ ID NO: 622 - DNA, 17 bp, synthetic construct
  misc_feature 1..17: note = Synthetic Oligonucleotide
  source 1..17: mol_type = other DNA
atagaaaacccaaatcc

SEQ ID NO: 623 - DNA, 17 bp, synthetic construct
  misc_feature 1..17: note = Synthetic Oligonucleotide
  source 1..17: mol_type = other DNA
ttatagaaaacccaaat

SEQ ID NO: 624 - DNA, 17 bp, synthetic construct
  misc_feature 1..17: note = Synthetic Oligonucleotide
  source 1..17: mol_type = other DNA
cttatagaaaacccaaa

SEQ ID NO: 625 - DNA, 17 bp, synthetic construct
  misc_feature 1..17: note = Synthetic Oligonucleotide
  source 1..17: mol_type = other DNA
ccttatagaaaacccaa

SEQ ID NO: 626 - DNA, 17 bp, synthetic construct
  misc_feature 1..17: note = Synthetic Oligonucleotide
  source 1..17: mol_type = other DNA
cccttatagaaaaccca

SEQ ID NO: 627 - DNA, 17 bp, synthetic construct
  misc_feature 1..17: note = Synthetic Oligonucleotide
  source 1..17: mol_type = other DNA
ccccttatagaaaaccc

SEQ ID NO: 628 - DNA, 17 bp, synthetic construct
  misc_feature 1..17: note = Synthetic Oligonucleotide
  source 1..17: mol_type = other DNA
accccttatagaaaacc

SEQ ID NO: 629 - DNA, 17 bp, synthetic construct
  misc_feature 1..17: note = Synthetic Oligonucleotide
  source 1..17: mol_type = other DNA
aaccccttatagaaaac

SEQ ID NO: 630 - DNA, 17 bp, synthetic construct
  misc_feature 1..17: note = Synthetic Oligonucleotide
  source 1..17: mol_type = other DNA
aaaccccttatagaaaa

SEQ ID NO: 631 - DNA, 17 bp, synthetic construct
  misc_feature 1..17: note = Synthetic Oligonucleotide
  source 1..17: mol_type = other DNA
gaaaccccttatagaaa

SEQ ID NO: 632 - DNA, 17 bp, synthetic construct
  misc_feature 1..17: note = Synthetic Oligonucleotide
  source 1..17: mol_type = other DNA
ggaaaccccttatagaa

SEQ ID NO: 633 - DNA, 17 bp, synthetic construct
  misc_feature 1..17: note = Synthetic Oligonucleotide
  source 1..17: mol_type = other DNA
aggaaaccccttataga

SEQ ID NO: 634 - DNA, 17 bp, synthetic construct
  misc_feature 1..17: note = Synthetic Oligonucleotide
  source 1..17: mol_type = other DNA
caggaaaccccttatag

SEQ ID NO: 635 - DNA, 17 bp, synthetic construct
  misc_feature 1..17: note = Synthetic Oligonucleotide
  source 1..17: mol_type = other DNA
gcaggaaaccccttata

SEQ ID NO: 636 - DNA, 17 bp, synthetic construct
  misc_feature 1..17: note = Synthetic Oligonucleotide
  source 1..17: mol_type = other DNA
agcaggaaaccccttat

SEQ ID NO: 637 - DNA, 17 bp, synthetic construct
  misc_feature 1..17: note = Synthetic Oligonucleotide
  source 1..17: mol_type = other DNA
cagcaggaaacccctta

SEQ ID NO: 638 - DNA, 17 bp, synthetic construct
  misc_feature 1..17: note = Synthetic Oligonucleotide
  source 1..17: mol_type = other DNA
ccagcaggaaacccctt

SEQ ID NO: 639 - DNA, 17 bp, synthetic construct
  misc_feature 1..17: note = Synthetic Oligonucleotide
  source 1..17: mol_type = other DNA
tccagcaggaaacccct

SEQ ID NO: 640 - DNA, 17 bp, synthetic construct
  misc_feature 1..17: note = Synthetic Oligonucleotide
  source 1..17: mol_type = other DNA
gtccagcaggaaacccc

SEQ ID NO: 641 - DNA, 17 bp, synthetic construct
  misc_feature 1..17: note = Synthetic Oligonucleotide
  source 1..17: mol_type = other DNA
tgtccagcaggaaaccc

SEQ ID NO: 642 - DNA, 17 bp, synthetic construct
  misc_feature 1..17: note = Synthetic Oligonucleotide
  source 1..17: mol_type = other DNA
ctgtccagcaggaaacc

SEQ ID NO: 643 - DNA, 17 bp, synthetic construct
  misc_feature 1..17: note = Synthetic Oligonucleotide
  source 1..17: mol_type = other DNA
cctgtccagcaggaaac

SEQ ID NO: 644 - DNA, 17 bp, synthetic construct
  misc_feature 1..17: note = Synthetic Oligonucleotide
  source 1..17: mol_type = other DNA
ccctgtccagcaggaaa

SEQ ID NO: 645 - DNA, 17 bp, synthetic construct
  misc_feature 1..17: note = Synthetic Oligonucleotide
  source 1..17: mol_type = other DNA
gcccctgtccagcagga

SEQ ID NO: 646 - DNA, 17 bp, synthetic construct
  misc_feature 1..17: note = Synthetic Oligonucleotide
  source 1..17: mol_type = other DNA
cgcccctgtccagcagg

SEQ ID NO: 647 - DNA, 17 bp, synthetic construct
  misc_feature 1..17: note = Synthetic Oligonucleotide
  source 1..17: mol_type = other DNA
acgcccctgtccagcag

SEQ ID NO: 648 - DNA, 17 bp, synthetic construct
  misc_feature 1..17: note = Synthetic Oligonucleotide
  source 1..17: mol_type = other DNA
cacgcccctgtccagca

SEQ ID NO: 649 - DNA, 17 bp, synthetic construct
  misc_feature 1..17: note = Synthetic Oligonucleotide
  source 1..17: mol_type = other DNA
ccacgcccctgtccagc

SEQ ID NO: 650 - DNA, 17 bp, synthetic construct
  misc_feature 1..17: note = Synthetic Oligonucleotide
  source 1..17: mol_type = other DNA
cccacgcccctgtccag

SEQ ID NO: 651 - DNA, 17 bp, synthetic construct
  misc_feature 1..17: note = Synthetic Oligonucleotide
  source 1..17: mol_type = other DNA
tcccacgcccctgtcca

SEQ ID NO: 652 - DNA, 17 bp, synthetic construct
  misc_feature 1..17: note = Synthetic Oligonucleotide
  source 1..17: mol_type = other DNA
atcccacgcccctgtcc

SEQ ID NO: 653 - DNA, 17 bp, synthetic construct
  misc_feature 1..17: note = Synthetic Oligonucleotide
  source 1..17: mol_type = other DNA
aatcccacgcccctgtc

SEQ ID NO: 654 - DNA, 17 bp, synthetic construct
  misc_feature 1..17: note = Synthetic Oligonucleotide
  source 1..17: mol_type = other DNA
caatcccacgcccctgt

SEQ ID NO: 655 - DNA, 17 bp, synthetic construct
  misc_feature 1..17: note = Synthetic Oligonucleotide
  source 1..17: mol_type = other DNA
tcaatcccacgcccctg

SEQ ID NO: 656 - DNA, 17 bp, synthetic construct
  misc_feature 1..17: note = Synthetic Oligonucleotide
  source 1..17: mol_type = other DNA
ttcaatcccacgcccct

SEQ ID NO: 657 - DNA, 17 bp, synthetic construct
  misc_feature 1..17: note = Synthetic Oligonucleotide
  source 1..17: mol_type = other DNA
attcaatcccacgcccc

SEQ ID NO: 658 - DNA, 17 bp, synthetic construct
  misc_feature 1..17: note = Synthetic Oligonucleotide
  source 1..17: mol_type = other DNA
aattcaatcccacgccc

SEQ ID NO: 659 - DNA, 17 bp, synthetic construct
  misc_feature 1..17: note = Synthetic Oligonucleotide
  source 1..17: mol_type = other DNA
taattcaatcccacgcc

SEQ ID NO: 660 - DNA, 17 bp, synthetic construct
  misc_feature 1..17: note = Synthetic Oligonucleotide
  source 1..17: mol_type = other DNA
ttaattcaatcccacgc

SEQ ID NO: 661 - DNA, 17 bp, synthetic construct
  misc_feature 1..17: note = Synthetic Oligonucleotide
  source 1..17: mol_type = other DNA
tttaattcaatcccacg

SEQ ID NO: 662 - DNA, 17 bp, synthetic construct
  misc_feature 1..17: note = Synthetic Oligonucleotide
  source 1..17: mol_type = other DNA
ttttaattcaatcccac

SEQ ID NO: 663 - DNA, 17 bp, synthetic construct
  misc_feature 1..17: note = Synthetic Oligonucleotide
  source 1..17: mol_type = other DNA
gttttaattcaatccca

SEQ ID NO: 664 - DNA, 17 bp, synthetic construct
  misc_feature 1..17: note = Synthetic Oligonucleotide
  source 1..17: mol_type = other DNA
tgttttaattcaatccc

SEQ ID NO: 665 - DNA, 17 bp, synthetic construct
  misc_feature 1..17: note = Synthetic Oligonucleotide
  source 1..17: mol_type = other DNA
ctgttttaattcaatcc

SEQ ID NO: 666 - DNA, 17 bp, synthetic construct
  misc_feature 1..17: note = Synthetic Oligonucleotide
  source 1..17: mol_type = other DNA
gctgttttaattcaatc

SEQ ID NO: 667 - DNA, 17 bp, synthetic construct
  misc_feature 1..17: note = Synthetic Oligonucleotide
  source 1..17: mol_type = other DNA
agctgttttaattcaat

SEQ ID NO: 668 - DNA, 17 bp, synthetic construct
  misc_feature 1..17: note = Synthetic Oligonucleotide
  source 1..17: mol_type = other DNA
cagctgttttaattcaa

SEQ ID NO: 669 - DNA, 17 bp, synthetic construct
  misc_feature 1..17: note = Synthetic Oligonucleotide
  source 1..17: mol_type = other DNA
gcagctgttttaattca

SEQ ID NO: 670 - DNA, 17 bp, synthetic construct
  misc_feature 1..17: note = Synthetic Oligonucleotide
  source 1..17: mol_type = other DNA
cgcagctgttttaattc

SEQ ID NO: 671 - DNA, 17 bp, synthetic construct
  misc_feature 1..17: note = Synthetic Oligonucleotide
  source 1..17: mol_type = other DNA
tcgcagctgttttaatt

SEQ ID NO: 672 - DNA, 17 bp, synthetic construct
  misc_feature 1..17: note = Synthetic Oligonucleotide
  source 1..17: mol_type = other DNA
gtcgcagctgttttaat

SEQ ID NO: 673 - DNA, 17 bp, synthetic construct
  misc_feature 1..17: note = Synthetic Oligonucleotide
  source 1..17: mol_type = other DNA
tgtcgcagctgttttaa

SEQ ID NO: 674 - DNA, 17 bp, synthetic construct
  misc_feature 1..17: note = Synthetic Oligonucleotide
  source 1..17: mol_type = other DNA
ttgtcgcagctgtttta

SEQ ID NO: 675 - DNA, 17 bp, synthetic construct
  misc_feature 1..17: note = Synthetic Oligonucleotide
  source 1..17: mol_type = other DNA
gttgtcgcagctgtttt

SEQ ID NO: 676 - DNA, 17 bp, synthetic construct
  misc_feature 1..17: note = Synthetic Oligonucleotide
  source 1..17: mol_type = other DNA
tgttgtcgcagctgttt

SEQ ID NO: 677 - DNA, 17 bp, synthetic construct
  misc_feature 1..17: note = Synthetic Oligonucleotide
  source 1..17: mol_type = other DNA
ttgttgtcgcagctgtt

SEQ ID NO: 678 - DNA, 17 bp, synthetic construct
  misc_feature 1..17: note = Synthetic Oligonucleotide
  source 1..17: mol_type = other DNA
tttgttgtcgcagctgt

SEQ ID NO: 679 - DNA, 17 bp, synthetic construct
  misc_feature 1..17: note = Synthetic Oligonucleotide
  source 1..17: mol_type = other DNA
ttttgttgtcgcagctg

SEQ ID NO: 680 - DNA, 17 bp, synthetic construct
  misc_feature 1..17: note = Synthetic Oligonucleotide
  source 1..17: mol_type = other DNA
tttttgttgtcgcagct

SEQ ID NO: 681 - DNA, 19 bp, synthetic construct
  misc_feature 1..19: note = Synthetic Oligonucleotide
  source 1..19: mol_type = other DNA
tagaaaacccaaatcctca

SEQ ID NO: 682 - DNA, 19 bp, synthetic construct
  misc_feature 1..19: note = Synthetic Oligonucleotide
  source 1..19: mol_type = other DNA
atagaaaacccaaatcctc

SEQ ID NO: 683 - DNA, 19 bp, synthetic construct
  misc_feature 1..19: note = Synthetic Oligonucleotide
  source 1..19: mol_type = other DNA
tatagaaaacccaaatcct

SEQ ID NO: 684 - DNA, 19 bp, synthetic construct
  misc_feature 1..19: note = Synthetic Oligonucleotide
  source 1..19: mol_type = other DNA
cttatagaaaacccaaatc

SEQ ID NO: 685 - DNA, 19 bp, synthetic construct
  misc_feature 1..19: note = Synthetic Oligonucleotide
  source 1..19: mol_type = other DNA
ccttatagaaaacccaaat

SEQ ID NO: 686 - DNA, 19 bp, synthetic construct
  misc_feature 1..19: note = Synthetic Oligonucleotide
  source 1..19: mol_type = other DNA
cccttatagaaaacccaaa

SEQ ID NO: 687 - DNA, 19 bp, synthetic construct
  misc_feature 1..19: note = Synthetic Oligonucleotide
  source 1..19: mol_type = other DNA
ccccttatagaaaacccaa

SEQ ID NO: 688 - DNA, 19 bp, synthetic construct
  misc_feature 1..19: note = Synthetic Oligonucleotide
  source 1..19: mol_type = other DNA
accccttatagaaaaccca

SEQ ID NO: 689 - DNA, 19 bp, synthetic construct
  misc_feature 1..19: note = Synthetic Oligonucleotide
  source 1..19: mol_type = other DNA
aaccccttatagaaaaccc

SEQ ID NO: 690 - DNA, 19 bp, synthetic construct
  misc_feature 1..19: note = Synthetic Oligonucleotide
  source 1..19: mol_type = other DNA
aaaccccttatagaaaacc

SEQ ID NO: 691 - DNA, 19 bp, synthetic construct
  misc_feature 1..19: note = Synthetic Oligonucleotide
  source 1..19: mol_type = other DNA
gaaaccccttatagaaaac

SEQ ID NO: 692 - DNA, 19 bp, synthetic construct
  misc_feature 1..19: note = Synthetic Oligonucleotide
  source 1..19: mol_type = other DNA
ggaaaccccttatagaaaa

SEQ ID NO: 693 - DNA, 19 bp, synthetic construct
  misc_feature 1..19: note = Synthetic Oligonucleotide
  source 1..19: mol_type = other DNA
aggaaaccccttatagaaa

SEQ ID NO: 694 - DNA, 19 bp, synthetic construct
  misc_feature 1..19: note = Synthetic Oligonucleotide
  source 1..19: mol_type = other DNA
caggaaaccccttatagaa

SEQ ID NO: 695 - DNA, 19 bp, synthetic construct
  misc_feature 1..19: note = Synthetic Oligonucleotide
  source 1..19: mol_type = other DNA
gcaggaaaccccttataga

SEQ ID NO: 696 - DNA, 19 bp, synthetic construct
  misc_feature 1..19: note = Synthetic Oligonucleotide
  source 1..19: mol_type = other DNA
agcaggaaaccccttatag

SEQ ID NO: 697 - DNA, 19 bp, synthetic construct
  misc_feature 1..19: note = Synthetic Oligonucleotide
  source 1..19: mol_type = other DNA
cagcaggaaaccccttata

SEQ ID NO: 698 - DNA, 19 bp, synthetic construct
  misc_feature 1..19: note = Synthetic Oligonucleotide
  source 1..19: mol_type = other DNA
ccagcaggaaaccccttat

SEQ ID NO: 699 - DNA, 19 bp, synthetic construct
  misc_feature 1..19: note = Synthetic Oligonucleotide
  source 1..19: mol_type = other DNA
tccagcaggaaacccctta

SEQ ID NO: 700 - DNA, 19 bp, synthetic construct
  misc_feature 1..19: note = Synthetic Oligonucleotide
  source 1..19: mol_type = other DNA
gtccagcaggaaacccctt

SEQ ID NO: 701 - DNA, 19 bp, synthetic construct
  misc_feature 1..19: note = Synthetic Oligonucleotide
  source 1..19: mol_type = other DNA
tgtccagcaggaaacccct

SEQ ID NO: 702 - DNA, 19 bp, synthetic construct
  misc_feature 1..19: note = Synthetic Oligonucleotide
  source 1..19: mol_type = other DNA
cctgtccagcaggaaaccc

SEQ ID NO: 703 - DNA, 19 bp, synthetic construct
  misc_feature 1..19: note = Synthetic Oligonucleotide
  source 1..19: mol_type = other DNA
ccctgtccagcaggaaacc

SEQ ID NO: 704 - DNA, 19 bp, synthetic construct
  misc_feature 1..19: note = Synthetic Oligonucleotide
  source 1..19: mol_type = other DNA
cccctgtccagcaggaaac

SEQ ID NO: 705 - DNA, 19 bp, synthetic construct
  misc_feature 1..19: note = Synthetic Oligonucleotide
  source 1..19: mol_type = other DNA
gcccctgtccagcaggaaa

SEQ ID NO: 706 - DNA, 19 bp, synthetic construct
  misc_feature 1..19: note = Synthetic Oligonucleotide
  source 1..19: mol_type = other DNA
acgcccctgtccagcagga

SEQ ID NO: 707 - DNA, 19 bp, synthetic construct
  misc_feature 1..19: note = Synthetic Oligonucleotide
  source 1..19: mol_type = other DNA
cacgcccctgtccagcagg

SEQ ID NO: 708 - DNA, 19 bp, synthetic construct
  misc_feature 1..19: note = Synthetic Oligonucleotide
  source 1..19: mol_type = other DNA
ccacgcccctgtccagcag

SEQ ID NO: 709 - DNA, 19 bp, synthetic construct
  misc_feature 1..19: note = Synthetic Oligonucleotide
  source 1..19: mol_type = other DNA
cccacgcccctgtccagca

SEQ ID NO: 710 - DNA, 19 bp, synthetic construct
  misc_feature 1..19: note = Synthetic Oligonucleotide
  source 1..19: mol_type = other DNA
tcccacgcccctgtccagc

SEQ ID NO: 711 - DNA, 19 bp, synthetic construct
  misc_feature 1..19: note = Synthetic Oligonucleotide
  source 1..19: mol_type = other DNA
atcccacgcccctgtccag

SEQ ID NO: 712 - DNA, 19 bp, synthetic construct
  misc_feature 1..19: note = Synthetic Oligonucleotide
  source 1..19: mol_type = other DNA
aatcccacgcccctgtcca

SEQ ID NO: 713 - DNA, 19 bp, synthetic construct
  misc_feature 1..19: note = Synthetic Oligonucleotide
  source 1..19: mol_type = other DNA
caatcccacgcccctgtcc

SEQ ID NO: 714 - DNA, 19 bp, synthetic construct
  misc_feature 1..19: note = Synthetic Oligonucleotide
  source 1..19: mol_type = other DNA
tcaatcccacgcccctgtc

SEQ ID NO: 715 - DNA, 19 bp, synthetic construct
  misc_feature 1..19: note = Synthetic Oligonucleotide
  source 1..19: mol_type = other DNA
ttcaatcccacgcccctgt

SEQ ID NO: 716 - DNA, 19 bp, synthetic construct
  misc_feature 1..19: note = Synthetic Oligonucleotide
  source 1..19: mol_type = other DNA
attcaatcccacgcccctg

SEQ ID NO: 717 - DNA, 19 bp, synthetic construct
  misc_feature 1..19: note = Synthetic Oligonucleotide
  source 1..19: mol_type = other DNA
aattcaatcccacgcccct

SEQ ID NO: 718 - DNA, 19 bp, synthetic construct
  misc_feature 1..19: note = Synthetic Oligonucleotide
  source 1..19: mol_type = other DNA
taattcaatcccacgcccc

SEQ ID NO: 719 - DNA, 19 bp, synthetic construct
  misc_feature 1..19: note = Synthetic Oligonucleotide
  source 1..19: mol_type = other DNA
ttaattcaatcccacgccc

SEQ ID NO: 720 - DNA, 19 bp, synthetic construct
  misc_feature 1..19: note = Synthetic Oligonucleotide
  source 1..19: mol_type = other DNA
tttaattcaatcccacgcc

SEQ ID NO: 721 - DNA, 19 bp, synthetic construct
  misc_feature 1..19: note = Synthetic Oligonucleotide
  source 1..19: mol_type = other DNA
ttttaattcaatcccacgc

SEQ ID NO: 722 - DNA, 19 bp, synthetic construct
  misc_feature 1..19: note = Synthetic Oligonucleotide
  source 1..19: mol_type = other DNA
gttttaattcaatcccacg

SEQ ID NO: 723 - DNA, 19 bp, synthetic construct
  misc_feature 1..19: note = Synthetic Oligonucleotide
  source 1..19: mol_type = other DNA
tgttttaattcaatcccac

SEQ ID NO: 724 - DNA, 19 bp, synthetic construct
  misc_feature 1..19: note = Synthetic Oligonucleotide
  source 1..19: mol_type = other DNA
ctgttttaattcaatccca

SEQ ID NO: 725 - DNA, 19 bp, synthetic construct
  misc_feature 1..19: note = Synthetic Oligonucleotide
  source 1..19: mol_type = other DNA
gctgttttaattcaatccc

SEQ ID NO: 726 - DNA, 19 bp, synthetic construct
  misc_feature 1..19: note = Synthetic Oligonucleotide
  source 1..19: mol_type = other DNA
agctgttttaattcaatcc

SEQ ID NO: 727 - DNA, 19 bp, synthetic construct
  misc_feature 1..19: note = Synthetic Oligonucleotide
  source 1..19: mol_type = other DNA
cagctgttttaattcaatc

SEQ ID NO: 728 - DNA, 19 bp, synthetic construct
  misc_feature 1..19: note = Synthetic Oligonucleotide
  source 1..19: mol_type = other DNA
gcagctgttttaattcaat

SEQ ID NO: 729 - DNA, 19 bp, synthetic construct
  misc_feature 1..19: note = Synthetic Oligonucleotide
  source 1..19: mol_type = other DNA
cgcagctgttttaattcaa

SEQ ID NO: 730 - DNA, 19 bp, synthetic construct
  misc_feature 1..19: note = Synthetic Oligonucleotide
  source 1..19: mol_type = other DNA
tcgcagctgttttaattca

SEQ ID NO: 731 - DNA, 19 bp, synthetic construct
  misc_feature 1..19: note = Synthetic Oligonucleotide
  source 1..19: mol_type = other DNA
gtcgcagctgttttaattc

SEQ ID NO: 732 - DNA, 19 bp, synthetic construct
  misc_feature 1..19: note = Synthetic Oligonucleotide
  source 1..19: mol_type = other DNA
tgtcgcagctgttttaatt

SEQ ID NO: 733 - DNA, 19 bp, synthetic construct
  misc_feature 1..19: note = Synthetic Oligonucleotide
  source 1..19: mol_type = other DNA
ttgtcgcagctgttttaat

SEQ ID NO: 734 - DNA, 19 bp, synthetic construct
  misc_feature 1..19: note = Synthetic Oligonucleotide
  source 1..19: mol_type = other DNA
gttgtcgcagctgttttaa

SEQ ID NO: 735 - DNA, 19 bp, synthetic construct
  misc_feature 1..19: note = Synthetic Oligonucleotide
  source 1..19: mol_type = other DNA
tgttgtcgcagctgtttta

SEQ ID NO: 736 - DNA, 19 bp, synthetic construct
  misc_feature 1..19: note = Synthetic Oligonucleotide
  source 1..19: mol_type = other DNA
ttgttgtcgcagctgtttt

SEQ ID NO: 737 - DNA, 19 bp, synthetic construct
  misc_feature 1..19: note = Synthetic Oligonucleotide
  source 1..19: mol_type = other DNA
tttgttgtcgcagctgttt

SEQ ID NO: 738 - DNA, 19 bp, synthetic construct
  misc_feature 1..19: note = Synthetic Oligonucleotide
  source 1..19: mol_type = other DNA
ttttgttgtcgcagctgtt

SEQ ID NO: 739 - DNA, 19 bp, synthetic construct
  misc_feature 1..19: note = Synthetic Oligonucleotide
  source 1..19: mol_type = other DNA
tttttgttgtcgcagctgt

SEQ ID NO: 740 - DNA, 18 bp, synthetic construct
  misc_feature 1..18: note = Synthetic Oligonucleotide
  source 1..18: mol_type = other DNA
ccccttatagaaaaccca

SEQ ID NO: 741 - DNA, 18 bp, synthetic construct
  misc_feature 1..18: note = Synthetic Oligonucleotide
  source 1..18: mol_type = other DNA
accccttatagaaaaccc

SEQ ID NO: 742 - DNA, 18 bp, synthetic construct
  misc_feature 1..18: note = Synthetic Oligonucleotide
  source 1..18: mol_type = other DNA
aaccccttatagaaaacc

SEQ ID NO: 743 - DNA, 18 bp, synthetic construct
  misc_feature 1..18: note = Synthetic Oligonucleotide
  source 1..18: mol_type = other DNA
aaaccccttatagaaaac

SEQ ID NO: 744 - DNA, 18 bp, synthetic construct
  misc_feature 1..18: note = Synthetic Oligonucleotide
  source 1..18: mol_type = other DNA
gaaaccccttatagaaaa

SEQ ID NO: 745 - DNA, 18 bp, synthetic construct
  misc_feature 1..18: note = Synthetic Oligonucleotide
  source 1..18: mol_type = other DNA
aggaaaccccttatagaa

SEQ ID NO: 746 - DNA, 18 bp, synthetic construct
  misc_feature 1..18: note = Synthetic Oligonucleotide
  source 1..18: mol_type = other DNA
caggaaaccccttataga

SEQ ID NO: 747 - DNA, 18 bp, synthetic construct
  misc_feature 1..18: note = Synthetic Oligonucleotide
  source 1..18: mol_type = other DNA
gcaggaaaccccttatag

SEQ ID NO: 748 - DNA, 18 bp, synthetic construct
  misc_feature 1..18: note = Synthetic Oligonucleotide
  source 1..18: mol_type = other DNA
agcaggaaaccccttata

SEQ ID NO: 749 - DNA, 18 bp, synthetic construct
  misc_feature 1..18: note = Synthetic Oligonucleotide
  source 1..18: mol_type = other DNA
cagcaggaaaccccttat

SEQ ID NO: 750 - DNA, 18 bp, synthetic construct
  misc_feature 1..18: note = Synthetic Oligonucleotide
  source 1..18: mol_type = other DNA
ccagcaggaaacccctta

SEQ ID NO: 751 - DNA, 18 bp, synthetic construct
  misc_feature 1..18: note = Synthetic Oligonucleotide
  source 1..18: mol_type = other DNA
tccagcaggaaacccctt

SEQ ID NO: 752 - DNA, 18 bp, synthetic construct
  misc_feature 1..18: note = Synthetic Oligonucleotide
  source 1..18: mol_type = other DNA
gtccagcaggaaacccct

SEQ ID NO: 753 - DNA, 18 bp, synthetic construct
  misc_feature 1..18: note = Synthetic Oligonucleotide
  source 1..18: mol_type = other DNA
tgtccagcaggaaacccc

SEQ ID NO: 754 - DNA, 18 bp, synthetic construct
  misc_feature 1..18: note = Synthetic Oligonucleotide
  source 1..18: mol_type = other DNA
ctgtccagcaggaaaccc

SEQ ID NO: 755 - DNA, 18 bp, synthetic construct
  misc_feature 1..18: note = Synthetic Oligonucleotide
  source 1..18: mol_type = other DNA
cctgtccagcaggaaacc

SEQ ID NO: 756 - DNA, 18 bp, synthetic construct
  misc_feature 1..18: note = Synthetic Oligonucleotide
  source 1..18: mol_type = other DNA
ccctgtccagcaggaaac

SEQ ID NO: 757 - DNA, 18 bp, synthetic construct
  misc_feature 1..18: note = Synthetic Oligonucleotide
  source 1..18: mol_type = other DNA
cccctgtccagcaggaaa

SEQ ID NO: 758 - DNA, 18 bp, synthetic construct
  misc_feature 1..18: note = Synthetic Oligonucleotide
  source 1..18: mol_type = other DNA
cgcccctgtccagcagga

SEQ ID NO: 759 - DNA, 18 bp, synthetic construct
  misc_feature 1..18: note = Synthetic Oligonucleotide
  source 1..18: mol_type = other DNA
acgcccctgtccagcagg

SEQ ID NO: 760 - DNA, 18 bp, synthetic construct
  misc_feature 1..18: note = Synthetic Oligonucleotide
  source 1..18: mol_type = other DNA
cacgcccctgtccagcag

SEQ ID NO: 761 - DNA, 18 bp, synthetic construct
  misc_feature 1..18: note = Synthetic Oligonucleotide
  source 1..18: mol_type = other DNA
ccacgcccctgtccagca

SEQ ID NO: 762 - DNA, 18 bp, synthetic construct
  misc_feature 1..18: note = Synthetic Oligonucleotide
  source 1..18: mol_type = other DNA
cccacgcccctgtccagc

SEQ ID NO: 763 - DNA, 18 bp, synthetic construct
  misc_feature 1..18: note = Synthetic Oligonucleotide
  source 1..18: mol_type = other DNA
tcccacgcccctgtccag

SEQ ID NO: 764 - DNA, 18 bp, synthetic construct
  misc_feature 1..18: note = Synthetic Oligonucleotide
  source 1..18: mol_type = other DNA
atcccacgcccctgtcca

SEQ ID NO: 765 - DNA, 18 bp, synthetic construct
  misc_feature 1..18: note = Synthetic Oligonucleotide
  source 1..18: mol_type = other DNA
aatcccacgcccctgtcc

SEQ ID NO: 766 - DNA, 18 bp, synthetic construct
  misc_feature 1..18: note = Synthetic Oligonucleotide
  source 1..18: mol_type = other DNA
caatcccacgcccctgtc

SEQ ID NO: 767 - DNA, 18 bp, synthetic construct
  misc_feature 1..18: note = Synthetic Oligonucleotide
  source 1..18: mol_type = other DNA
tcaatcccacgcccctgt

SEQ ID NO: 768 - DNA, 18 bp, synthetic construct
  misc_feature 1..18: note = Synthetic Oligonucleotide
  source 1..18: mol_type = other DNA
ttcaatcccacgcccctg

SEQ ID NO: 769 - DNA, 18 bp, synthetic construct
  misc_feature 1..18: note = Synthetic Oligonucleotide
  source 1..18: mol_type = other DNA
attcaatcccacgcccct

SEQ ID NO: 770 - DNA, 18 bp, synthetic construct
  misc_feature 1..18: note = Synthetic Oligonucleotide
  source 1..18: mol_type = other DNA
aattcaatcccacgcccc

SEQ ID NO: 771 - DNA, 18 bp, synthetic construct
  misc_feature 1..18: note = Synthetic Oligonucleotide
  source 1..18: mol_type = other DNA
taattcaatcccacgccc

SEQ ID NO: 772 - DNA, 18 bp, synthetic construct
  misc_feature 1..18: note = Synthetic Oligonucleotide
  source 1..18: mol_type = other DNA
ttaattcaatcccacgcc

SEQ ID NO: 773 - DNA, 18 bp, synthetic construct
  misc_feature 1..18: note = Synthetic Oligonucleotide
  source 1..18: mol_type = other DNA
tttaattcaatcccacgc

SEQ ID NO: 774 - DNA, 18 bp, synthetic construct
  misc_feature 1..18: note = Synthetic Oligonucleotide
  source 1..18: mol_type = other DNA
ttttaattcaatcccacg

SEQ ID NO: 775 - DNA, 18 bp, synthetic construct
  misc_feature 1..18: note = Synthetic Oligonucleotide
  source 1..18: mol_type = other DNA
gttttaattcaatcccac

SEQ ID NO: 776 - DNA, 18 bp, synthetic construct
  misc_feature 1..18: note = Synthetic Oligonucleotide
  source 1..18: mol_type = other DNA
tgttttaattcaatccca

SEQ ID NO: 777 - DNA, 18 bp, synthetic construct
  misc_feature 1..18: note = Synthetic Oligonucleotide
  source 1..18: mol_type = other DNA
ctgttttaattcaatccc

SEQ ID NO: 778 - DNA, 18 bp, synthetic construct
  misc_feature 1..18: note = Synthetic Oligonucleotide
  source 1..18: mol_type = other DNA
gctgttttaattcaatcc

SEQ ID NO: 779 - DNA, 18 bp, synthetic construct
  misc_feature 1..18: note = Synthetic Oligonucleotide
  source 1..18: mol_type = other DNA
agctgttttaattcaatc

SEQ ID NO: 780 - DNA, 18 bp, synthetic construct
  misc_feature 1..18: note = Synthetic Oligonucleotide
  source 1..18: mol_type = other DNA
cagctgttttaattcaat

SEQ ID NO: 781 - DNA, 18 bp, synthetic construct
  misc_feature 1..18: note = Synthetic Oligonucleotide
  source 1..18: mol_type = other DNA
gcagctgttttaattcaa

SEQ ID NO: 782 - DNA, 18 bp, synthetic construct
  misc_feature 1..18: note = Synthetic Oligonucleotide
  source 1..18: mol_type = other DNA
cgcagctgttttaattca

SEQ ID NO: 783 - DNA, 18 bp, synthetic construct
  misc_feature 1..18: note = Synthetic Oligonucleotide
  source 1..18: mol_type = other DNA
tcgcagctgttttaattc

SEQ ID NO: 784 - DNA, 18 bp, synthetic construct
  misc_feature 1..18: note = Synthetic Oligonucleotide
  source 1..18: mol_type = other DNA
gtcgcagctgttttaatt

SEQ ID NO: 785 - DNA, 18 bp, synthetic construct
  misc_feature 1..18: note = Synthetic Oligonucleotide
  source 1..18: mol_type = other DNA
tagaaaacccaaatcctc

SEQ ID NO: 786 - DNA, 18 bp, synthetic construct
  misc_feature 1..18: note = Synthetic Oligonucleotide
  source 1..18: mol_type = other DNA
atagaaaacccaaatcct

SEQ ID NO: 787 - DNA, 18 bp, synthetic construct
  misc_feature 1..18: note = Synthetic Oligonucleotide
  source 1..18: mol_type = other DNA
tatagaaaacccaaatcc

SEQ ID NO: 788 - DNA, 18 bp, synthetic construct
  misc_feature 1..18: note = Synthetic Oligonucleotide
  source 1..18: mol_type = other DNA
ttatagaaaacccaaatc

SEQ ID NO: 789 - DNA, 18 bp, synthetic construct
  misc_feature 1..18: note = Synthetic Oligonucleotide
  source 1..18: mol_type = other DNA
cttatagaaaacccaaat

SEQ ID NO: 790 - DNA, 18 bp, synthetic construct
  misc_feature 1..18: note = Synthetic Oligonucleotide
  source 1..18: mol_type = other DNA
ccttatagaaaacccaaa

SEQ ID NO: 791 - DNA, 18 bp, synthetic construct
  misc_feature 1..18: note = Synthetic Oligonucleotide
  source 1..18: mol_type = other DNA
cccttatagaaaacccaa

SEQ ID NO: 792 - DNA, 18 bp, synthetic construct
  misc_feature 1..18: note = Synthetic Oligonucleotide
  source 1..18: mol_type = other DNA
tgtcgcagctgttttaat

SEQ ID NO: 793 - DNA, 18 bp, synthetic construct
  misc_feature 1..18: note = Synthetic Oligonucleotide
  source 1..18: mol_type = other DNA
ttgtcgcagctgttttaa

SEQ ID NO: 794 - DNA, 18 bp, synthetic construct
  misc_feature 1..18: note = Synthetic Oligonucleotide
  source 1..18: mol_type = other DNA
gttgtcgcagctgtttta

SEQ ID NO: 795 - DNA, 18 bp, synthetic construct
  misc_feature 1..18: note = Synthetic Oligonucleotide
  source 1..18: mol_type = other DNA
tgttgtcgcagctgtttt

SEQ ID NO: 796 - DNA, 18 bp, synthetic construct
  misc_feature 1..18: note = Synthetic Oligonucleotide
  source 1..18: mol_type = other DNA
ttgttgtcgcagctgttt

SEQ ID NO: 797 - DNA, 18 bp, synthetic construct
  misc_feature 1..18: note = Synthetic Oligonucleotide
  source 1..18: mol_type = other DNA
tttgttgtcgcagctgtt

SEQ ID NO: 798 - DNA, 18 bp, synthetic construct
  misc_feature 1..18: note = Synthetic Oligonucleotide
  source 1..18: mol_type = other DNA
ttttgttgtcgcagctgt

SEQ ID NO: 799 - DNA, 18 bp, synthetic construct
  misc_feature 1..18: note = Synthetic Oligonucleotide
  source 1..18: mol_type = other DNA
tttttgttgtcgcagctg

SEQ ID NO: 800 - DNA, 16 bp, synthetic construct
  misc_feature 1..16: note = Synthetic Oligonucleotide
  source 1..16: mol_type = other DNA
aaaacccaaatcctca

SEQ ID NO: 801 - DNA, 16 bp, synthetic construct
  misc_feature 1..16: note = Synthetic Oligonucleotide
  source 1..16: mol_type = other DNA
agaaaacccaaatcct

SEQ ID NO: 802 - DNA, 16 bp, synthetic construct
  misc_feature 1..16: note = Synthetic Oligonucleotide
  source 1..16: mol_type = other DNA
tatagaaaacccaaat

SEQ ID NO: 803 - DNA, 16 bp, synthetic construct
  misc_feature 1..16: note = Synthetic Oligonucleotide
  source 1..16: mol_type = other DNA
ttatagaaaacccaaa

SEQ ID NO: 804 - DNA, 20 bp, synthetic construct
  misc_feature 1..20: note = Synthetic Oligonucleotide
  source 1..20: mol_type = other DNA
gcataagagggtaccagctg

SEQ ID NO: 805 - DNA, 20 bp, synthetic construct
  misc_feature 1..20: note = Synthetic Oligonucleotide
  source 1..20: mol_type = other DNA
gtcctttagccagggcagca

SEQ ID NO: 806 - DNA, 20 bp, synthetic construct
  misc_feature 1..20: note = Synthetic Oligonucleotide
  source 1..20: mol_type = other DNA
tccacccatgttgtgcaagc

SEQ ID NO: 807 - DNA, 20 bp, synthetic construct
  misc_feature 1..20: note = Synthetic Oligonucleotide
  source 1..20: mol_type = other DNA
ccacaccatgccacagagac

SEQ ID NO: 808 - DNA, 20 bp, synthetic construct
  misc_feature 1..20: note = Synthetic Oligonucleotide
  source 1..20: mol_type = other DNA
ttccgagtcaggctcttccc

SEQ ID NO: 809 - DNA, 20 bp, synthetic construct
  misc_feature 1..20: note = Synthetic Oligonucleotide
  source 1..20: mol_type = other DNA
ccttccctgaaggttcctcc

SEQ ID NO: 810 - DNA, 21 bp, synthetic construct
  misc_feature 1..21: note = Primer
  source 1..21: mol_type = other DNA
agtctctgtggcatggtttgg

SEQ ID NO: 811 - DNA, 21 bp, synthetic construct
  misc_feature 1..21: note = primer
  source 1..21: mol_type = other DNA
gggcgaatgactgagatcttg

SEQ ID NO: 812 - DNA, 27 bp, synthetic construct
  misc_feature 1..27: note = Probe
  source 1..27: mol_type = other DNA
taccgattaccacaagcaaccatggca

SEQ ID NO: 813 - DNA, 22 bp, synthetic construct
  misc_feature 1..22: note = Primer
  source 1..22: mol_type = other DNA
cgaagcagctcaatgaaatcaa

SEQ ID NO: 814 - DNA, 18 bp, synthetic construct
  misc_feature 1..18: note = Primer
  source 1..18: mol_type = other DNA
tgcctggagggccttctt

SEQ ID NO: 815 - DNA, 18 bp, synthetic construct
  misc_feature 1..18: note = Probe
  source 1..18: mol_type = other DNA
agaccacaagttgaagtc

SEQ ID NO: 816 - DNA, 20 bp, synthetic construct
  misc_feature 1..20: note = Primer
  source 1..20: mol_type = other DNA
gggcaaacagcaatttgtga

SEQ ID NO: 817 - DNA, 20 bp, synthetic construct
  misc_feature 1..20: note = Primer
  source 1..20: mol_type = other DNA
tggctacccaccttccttgt

SEQ ID NO: 818 - DNA, 28 bp, synthetic construct
  misc_feature 1..28: note = Probe
  source 1..28: mol_type = other DNA
ctggatactgtcccaatcccggtattcc

SEQ ID NO: 819 - DNA, 22 bp, synthetic construct
  misc_feature 1..22: note = Synthetic Oligonucleotide
  source 1..22: mol_type = other DNA
cgaagaagctcagtgaaatcaa

SEQ ID NO: 820 - DNA, 18 bp, synthetic construct
  misc_feature 1..18: note = Synthetic Oligonucleotide
  source 1..18: mol_type = other DNA
tgcctggagggccctctt

SEQ ID NO: 821 - DNA, 20 bp, synthetic construct
  misc_feature 1..20: note = Synthetic Oligonucleotide
  source 1..20: mol_type = other DNA
agcttcttgtccagctttat

SEQ ID NO: 822 - DNA, 21 bp, synthetic construct
  misc_feature 1..21: note = Synthetic Oligonucleotide
  source 1..21: mol_type = other DNA
agcttcttgtccagctttata

SEQ ID NO: 823 - DNA, 14 bp, synthetic construct
  misc_feature 1..14: note = Synthetic Oligonucleotide
  source 1..14: mol_type = other DNA
tcagtcatgacttc

SEQ ID NO: 824 - DNA, 15 bp, synthetic construct
  misc_feature 1..15: note = Synthetic Oligonucleotide
  source 1..15: mol_type = other DNA
tcagtcatgacttca

SEQ ID NO: 825 - DNA, 20 bp, synthetic construct
  misc_feature 1..20: note = Synthetic Oligonucleotide
  source 1..20: mol_type = other DNA
gctgattagagagaggtccc

SEQ ID NO: 826 - DNA, 20 bp, synthetic construct
  misc_feature 1..20: note = Synthetic Oligonucleotide
  source 1..20: mol_type = other DNA
tcccatttcaggagacctgg

SEQ ID NO: 827 - DNA, 15 bp, synthetic construct
  misc_feature 1..15: note = Synthetic Oligonucleotide
  source 1..15: mol_type = other DNA
atcagtcatgacttc

SEQ ID NO: 828 - DNA, 20 bp, synthetic construct
  misc_feature 1..20: note = Synthetic Oligonucleotide
  source 1..20: mol_type = other DNA
cggtgcaaggcttaggaatt

SEQ ID NO: 829 - DNA, 20 bp, synthetic construct
  misc_feature 1..20: note = Synthetic Oligonucleotide
  source 1..20: mol_type = other DNA
gcttcagtcatgacttcctt

SEQ ID NO: 830 - DNA, 21 bp, synthetic construct
  misc_feature 1..21: note = Synthetic Oligonucleotide
  source 1..21: mol_type = other DNA
gcttcagtcatgacttcctta

SEQ ID NO: 831 - DNA, 21 bp, synthetic construct
  misc_feature 1..21: note = Synthetic Oligonucleotide
  source 1..21: mol_type = other DNA
agcttcagtcatgacttcctt

SEQ ID NO: 832 - DNA, 20 bp, synthetic construct
  misc_feature 1..20: note = Synthetic Oligonucleotide
  source 1..20: mol_type = other DNA
tggtaatccactttcagagg

SEQ ID NO: 833 - DNA, 21 bp, synthetic construct
  misc_feature 1..21: note = Synthetic Oligonucleotide
  source 1..21: mol_type = other DNA
tggtaatccactttcagagga

SEQ ID NO: 834 - DNA, 21 bp, synthetic construct
  misc_feature 1..21: note = Synthetic Oligonucleotide
  source 1..21: mol_type = other DNA
tgcttcagtcatgacttcctt

SEQ ID NO: 835 - DNA, 20 bp, synthetic construct
  misc_feature 1..20: note = Synthetic Oligonucleotide
  source 1..20: mol_type = other DNA
cactgatttttgcccaggat

SEQ ID NO: 836 - DNA, 21 bp, synthetic construct
  misc_feature 1..21: note = Synthetic Oligonucleotide
  source 1..21: mol_type = other DNA
cactgatttttgcccaggata

SEQ ID NO: 837 - DNA, 21 bp, synthetic construct
  misc_feature 1..21: note = Synthetic Oligonucleotide
  source 1..21: mol_type = other DNA
aagcttcttgtccagctttat

SEQ ID NO: 838 - DNA, 20 bp, synthetic construct
  misc_feature 1..20: note = Synthetic Oligonucleotide
  source 1..20: mol_type = other DNA
acccaattcagaaggaagga

SEQ ID NO: 839 - DNA, 21 bp, synthetic construct
  misc_feature 1..21: note = Synthetic Oligonucleotide
  source 1..21: mol_type = other DNA
acccaattcagaaggaaggaa

SEQ ID NO: 840 - DNA, 21 bp, synthetic construct
  misc_feature 1..21: note = Synthetic Oligonucleotide
  source 1..21: mol_type = other DNA
aacccaattcagaaggaagga

SEQ ID NO: 841 - DNA, 21 bp, synthetic construct
  misc_feature 1..21: note = Synthetic Oligonucleotide
  source 1..21: mol_type = other DNA
atggtaatccactttcagagg

SEQ ID NO: 842 - DNA, 20 bp, synthetic construct
  misc_feature 1..20: note = Synthetic Oligonucleotide
  source 1..20: mol_type = other DNA
tcttggttacatgaaatccc

SEQ ID NO: 843 - DNA, 21 bp, synthetic construct
  misc_feature 1..21: note = Synthetic Oligonucleotide
  source 1..21: mol_type = other DNA
tcttggttacatgaaatccca

SEQ ID NO: 844 - DNA, 20 bp, synthetic construct
  misc_feature 1..20: note = Synthetic Oligonucleotide
  source 1..20: mol_type = other DNA
attcactttcataatgctgg

SEQ ID NO: 845 - DNA, 21 bp, synthetic construct
  misc_feature 1..21: note = Synthetic Oligonucleotide
  source 1..21: mol_type = other DNA
attcactttcataatgctgga

SEQ ID NO: 846 - DNA, 21 bp, synthetic construct
  misc_feature 1..21: note = Synthetic Oligonucleotide
  source 1..21: mol_type = other DNA
atcttggttacatgaaatccc

SEQ ID NO: 847 - DNA, 20 bp, synthetic construct
  misc_feature 1..20: note = Synthetic Oligonucleotide
  source 1..20: mol_type = other DNA
tgctccgttggtgcttgttc

SEQ ID NO: 848 - DNA, 20 bp, synthetic construct
  misc_feature 1..20: note = Synthetic Oligonucleotide
  source 1..20: mol_type = other DNA
atgcatggtgatgcttctga

SEQ ID NO: 849 - DNA, 20 bp, synthetic construct
  misc_feature 1..20: note = Synthetic Oligonucleotide
  source 1..20: mol_type = other DNA
cagctttattagggacagca

SEQ ID NO: 850 - DNA, 21 bp, synthetic construct
  misc_feature 1..21: note = Synthetic Oligonucleotide
  source 1..21: mol_type = other DNA
cagctttattagggacagcaa

SEQ ID NO: 851 - DNA, 21 bp, synthetic construct
  misc_feature 1..21: note = Synthetic Oligonucleotide
  source 1..21: mol_type = other DNA
acagctttattagggacagca

SEQ ID NO: 852 - DNA, 16 bp, synthetic construct
  misc_feature 1..16: note = Synthetic Oligonucleotide
  source 1..16: mol_type = other DNA
ttcagtcatgacttcc

SEQ ID NO: 853 - DNA, 20 bp, synthetic construct
  misc_feature 1..20: note = Synthetic Oligonucleotide
  misc_feature 1..5: note = Bases at these positions are RNA
  misc_feature 16..20: note = Bases at these positions are RNA
  source 1..20: mol_type = other DNA
  misc_feature 6..15: note = Bases at these positions are DNA
gcttcagtcatgacttcctt

SEQ ID NO: 854 - DNA, 21 bp, synthetic construct
  misc_feature 1..21: note = Synthetic Oligonucleotide
  source 1..21: mol_type = other DNA
tgctccgttggtgcttgttca